(19) United States
(12) Patent Application Publication
(10) Pub. No.: US 2025/0244668 A1
(43) Pub. Date: Jul. 31, 2025

(54) ONIUM SALT MONOMER, POLYMER, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventors: Masahiro Fukushima, Joetsu-shi (JP); Takayuki Fujiwara, Joetsu-shi (JP); Tomonari Noguchi, Joetsu-shi (JP); Junya Kimura, Joetsu-shi (JP)

(73) Assignee: Shin-Etsu Chemical Co., Ltd., Tokyo (JP)

(21) Appl. No.: 19/036,698

(22) Filed: Jan. 24, 2025

(30) Foreign Application Priority Data
Jan. 31, 2024 (JP) 2024-012406

(51) Int. Cl. G03F 7/029; G03F 7/004; G03F 7/039; G03F 7/20

(52) U.S. Cl. CPC G03F 7/029; G03F 7/0045; G03F 7/0048; G03F 7/0392; G03F 7/2006

(57) ABSTRACT

An onium salt monomer consists of a fluorosulfonic acid anion containing an aromatic ring structure substituted with a polymerizable group and iodine and a triarylsulfonium cation containing iodine and fluorine. A resist composition comprising a polymer comprising repeat units derived from the monomer exhibits a high solvent solubility, high sensitivity and high contrast and forms a small-size pattern with satisfactory exposure latitude, edge roughness, dimension uniformity and depth of focus when processed by photolithography using high-energy radiation, as well as collapse resistance and etch resistance.

### CROSS-REFERENCE TO RELATED APPLICATIONS

This non-provisional application claims priority under 35 U.S.C. § 119(a) on Patent Application No. 2024-012406 filed in Japan on Jan. 31, 2024, the entire contents of which are hereby incorporated by reference.

### TECHNICAL FIELD

This invention relates to an onium salt monomer, polymer, chemically amplified resist composition, and pattern forming process.

### BACKGROUND ART

To meet the demand for higher integration density and operating speed of LSIs, the effort to reduce the pattern rule is in rapid progress. The wide-spreading flash memory market and the demand for increased storage capacities drive forward the miniaturization technology. As the advanced miniaturization technology, manufacturing of microelectronic devices at the 65-nm node by the ArF lithography has been implemented in a mass scale. Manufacturing of 45-nm node devices by the next generation ArF immersion lithography is approaching to the verge of high-volume application. The candidates for the next generation 32-nm node include ultra-high NA lens immersion lithography using a liquid having a higher refractive index than water in combination with a high refractive index lens and a high refractive index resist film, EUV lithography of wavelength 13.5 nm, and double patterning version of the ArF lithography, on which active research efforts have been made.

As the pattern feature size is reduced, approaching to the diffraction limit of light, light contrast lowers. In the case of positive resist film, a lowering of light contrast leads to reductions of resolution and focus margin of hole and trench patterns.

As the pattern feature size is reduced, the edge roughness (LWR) of line patterns and the critical dimension uniformity (CDU) of hole patterns are regarded significant. It is pointed out that these factors are affected by the segregation or agglomeration of a base polymer and acid generator and the diffusion of generated acid. There is a tendency that as the resist film becomes thinner, LWR becomes greater. A film thickness reduction to comply with the progress of size reduction causes a degradation of LWR, which becomes a serious problem.

The EUV lithography resist must meet high sensitivity, high resolution and low LWR at the same time. As the acid diffusion distance is reduced, LWR is reduced, but sensitivity becomes lower. For example, as the PEB temperature is lowered, the outcome is a reduced LWR, but a lower sensitivity. As the amount of quencher added is increased, the outcome is a reduced LWR, but a lower sensitivity. It is necessary to overcome the tradeoff relation between sensitivity and LWR.

With the aim to suppress acid diffusion, Patent Document 1 discloses a resist compound comprising repeat units derived from an onium salt of a polymerizable unsaturated bond-containing sulfonic acid. Since the so-called polymer-bound acid generator is capable of generating a polymer type sulfonic acid upon exposure, it is characterized by a very short distance of acid diffusion. Sensitivity may be enhanced by increasing a proportion of the acid generator. In the case of addition type acid generators, as the amount of acid generator added is increased, a higher sensitivity is achievable, but the acid diffusion distance is also increased. Since the acid diffusion is non-uniform, an increase of acid diffusion leads to degraded LWR or CDU. With respect to a balance of sensitivity, LWR and CDU, the polymer-bound acid generator is regarded as having a high capability.

Since iodine atoms are highly absorptive to EUV of wavelength 13.5 nm, they generate secondary electrons upon light exposure. This effect is noteworthy in the EUV lithography. Patent Document 2 describes a photoacid generator having an iodized anion. Patent Document 3 describes a photoacid generator having an iodized anion and containing a polymerizable group. Patent Document 4 describes a photoacid generator consisting of a cation and an anion, both having iodine introduced therein. Although the lithography performance is improved to some extent, the organic solvent solubility of iodine-containing compounds is not so high, accompanied with a concern about precipitation in the solvent.

Patent Document 5 discloses a photoacid generator having a cation containing a plurality of fluorine atoms. The inclusion of plural fluorine atoms is effective for improving the solvent solubility of the photoacid generator, but is still insufficient from the aspect of EUV absorption. There is left room for further improvement.

Patent Documents 6 to 10 disclose a photoacid generator and quencher (acid diffusion inhibitor) having a cation containing iodine or fluorine. Patent Document 11 discloses an onium salt monomer having a cation containing iodine and a polymerizable group. These developing efforts are successful in improving the performance of resist materials, but is still unsatisfactory from the aspect of acid diffusion control. It is desired to have a resist material which is useful in forming small-size patterns.

### CITATION LIST

Patent Document 1: JP 4425776
Patent Document 2: JP 6720926
Patent Document 3: JP 6973274
Patent Document 4: JP 7041204
Patent Document 5: JP 7389562
Patent Document 6: JP-A 2021-123579
Patent Document 7: JP-A 2021-123580
Patent Document 8: JP-A 2022-123839
Patent Document 9: JP-A 2023-088869
Patent Document 10: JP-A 2023-088870
Patent Document 11: JP-A 2022-028615

### SUMMARY OF THE INVENTION

It is desired to develop an acid-catalyzed or chemically amplified resist composition exhibiting a high sensitivity, reduced LWR of line patterns, improved CDU of hole patterns, and etch resistance after pattern formation.

An object of the invention is to provide an onium salt monomer, a polymer comprising repeat units derived from the monomer, and a chemically amplified resist composition comprising the polymer, the resist composition, when processed by photolithography using high-energy radiation such as KrF excimer laser, ArF excimer laser, EB or EUV, exhibiting a satisfactory solvent solubility, high sensitivity, high contrast, and improved lithography properties including LWR, CDU, EL, and DOF as well as resistance to pattern collapse and etch resistance in small-size pattern formation. Another object of the invention is to provide a pattern forming process using the resist composition.

The inventors have found that a polymer comprising repeat units derived from an onium salt monomer consisting of a fluorosulfonic acid anion containing an aromatic ring structure substituted with a polymerizable group and iodine and a triarylsulfonium cation containing iodine and fluorine has satisfactory solvent solubility; and that using the polymer as a polymer-bound photoacid generator, a chemically amplified resist composition having a high sensitivity, high contrast, high resolution, and improved lithography properties including LWR and CDU, as well as etch resistance after pattern formation is obtained.

In one aspect, the invention provides an onium salt monomer consisting of an anion having the formula (a1) and a cation having the formula (a2).

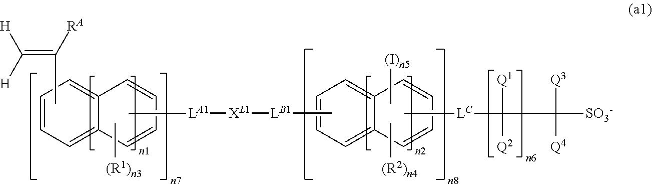

Herein n1 is 0 or 1, n2 is 0 or 1, n3 is an integer of 0 to 4, n4 is an integer of 0 to 4, n5 is an integer of 1 to 6, n4+n5 is from 1 to 4 when n2=0, n4+n5 is from 1 to 6 when n2=1, n6 is an integer of 0 to 4, n7 is 0 or 1, n8 is 0 or 1, excluding that n7 and n8 are 0 at the same time,
R^{A} is hydrogen, fluorine, methyl or trifluoromethyl,
R¹ is halogen, nitro group, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom, with the proviso that when n3=2, 3 or 4 and n7=1, a plurality of R¹ may be identical or different and a plurality of R¹ may bond together to form a ring with the carbon atoms to which they are attached, and that when n8=0, n7 is 1 and n3 is 1, 2, 3 or 4, and at least one of R¹ is iodine,
R² is halogen exclusive of iodine, nitro group, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom, with the proviso that a plurality of R² may be identical or different and a plurality of R² may bond together to form a ring with the carbon atoms to which they are attached, when n4=2, 3 or 4 and n8=1,
L^{A1}, L^{B1} and L^{C} are each independently a single bond, ether bond, ester bond, sulfonate ester bond, amide bond, sulfonamide bond, carbonate bond or carbamate bond,
X^{L1} is a single bond or a C₁-C₄₀ hydrocarbylene group which may contain a heteroatom,
Q¹ and Q² are each independently hydrogen, fluorine or a C₁-C₆ fluorinated saturated hydrocarbyl group,
Q³ and Q⁴ are each independently fluorine or a C₁-C₆ fluorinated saturated hydrocarbyl group.

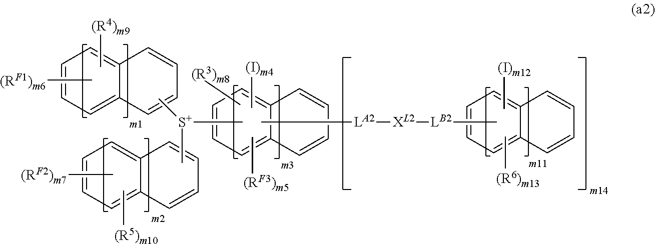

Herein m1 is 0 or 1, m2 is 0 or 1, m3 is 0 or 1, m4 is an integer of 0 to 4, m5 is an integer of 0 to 4, m6 is an integer of 0 to 6, m7 is an integer of 0 to 6, m8 is an integer of 0 to 2, m9 is an integer of 0 to 2, m10 is an integer of 0 to 2, m11 is 0 or 1, m12 is an integer of 0 to 4, m13 is an integer of 0 to 2, m14 is an integer of 0 to 2, m6+m9 is from 0 to 4 when m1=0, m6+m9 is from 0 to 6 when m1=1, m7+m10 is from 0 to 4 when m2=0, m7+m10 is from 0 to 6 when m2=1, m4+m5+m8+m14 is from 1 to 4 when m3=0, m4+m5+m8+m14 is from 1 to 6 when m3=1, m12+m13 is from 0 to 4 when m11=0, m12+m13 is from 0 to 6 when m11=1, m4+m12 is 1 or greater,
R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group,
with the proviso that a plurality of R^{F1} may be identical or different when m6 is 2 or more, a plurality of R^{F2} may be identical or different when m7 is 2 or more, and a plurality of R^{F3} may be identical or different when m5 is 2 or more,
R³ to R⁶ are each independently halogen exclusive of iodine and fluorine, nitro, cyano, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom, with the proviso that two R³ may be identical or different and two R³ may bond together to form a ring with the carbon atoms to which they are attached, when m8=2, two R⁴ may be identical or different and two R⁴ may bond together to form a ring with the carbon atoms to which they are attached, when m9=2, two R⁵ may be identical or different and two R⁵ may bond together to form a ring with the carbon atoms to which they are attached, when m10=2, two R⁶ may be identical or different and two R⁶ may bond together to form a ring with the carbon atoms to which they are attached, when m13=2,
the aromatic rings directly bonded to S⁺ in the sulfonium cation may bond together to form a ring with S⁺,
L^{A2} and L^{B2} are each independently a single bond, ether bond, ester bond, amide bond, sulfonate ester bond, sulfonamide bond, carbonate bond or carbamate bond, and
X^{L2} is a single bond or a C₁-C₄₀ hydrocarbylene group which may contain a heteroatom.

In a preferred embodiment, the anion has the formula (a1-1):

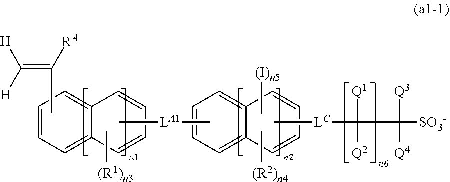

wherein n1 to n6, R^{A}, R¹, R², L^{A1}, L^{C} and Q¹ to Q⁴ are as defined above.

In a more preferred embodiment, the anion has the formula (a1-2):

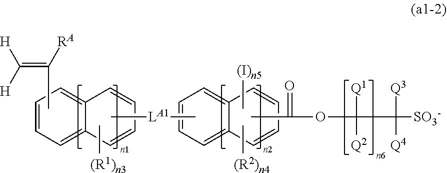

wherein n1 to n6, R^{A}, R¹, R², L^{A1} and Q¹ to Q⁴ are as defined above.

In a preferred embodiment, the cation has the formula (a2-1):

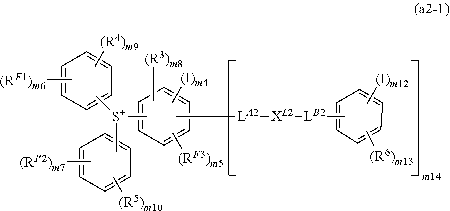

wherein m4 to m10, m12 to m14, R^{F1} to R^{F3}, R³ to R⁶, L^{A2}, L^{B2} and X^{L2} are as defined above.

In a more preferred embodiment, the cation has the formula (a2-2):

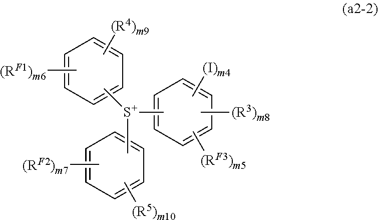

wherein m4 to m10, R^{F1} to R^{F3}, and R³ to R⁵ are as defined above.

In another aspect, the invention provides a monomeric photoacid generator comprising the onium salt monomer defined herein.

In a further aspect, the invention provides a polymer comprising repeat units derived from the monomeric photoacid generator defined above.

The polymer may further comprise repeat units having the formula (b1) or (b2).

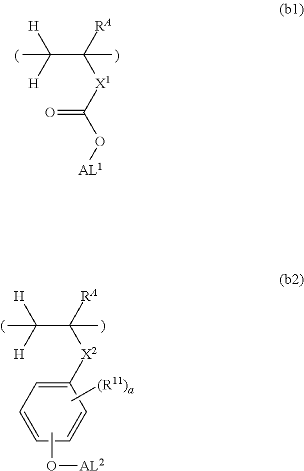

Herein R^{A} is each independently hydrogen, fluorine, methyl or trifluoromethyl,
X¹ is a single bond, phenylene group, naphthylene group, *—C(═O)—O—X¹¹— or *—C(═O)—NH—X¹¹—, the phenylene and naphthylene groups may be substituted with hydroxy, nitro, cyano, a C₁-C₁₀ alkoxy moiety which may contain a heteroatom, or halogen, X¹¹ is a C₁-C₁₀ saturated hydrocarbylene group, phenylene group or naphthylene group, the saturated hydrocarbylene group may contain hydroxy, ether bond, ester bond or lactone ring,
X² is a single bond, *—C(═O)—O— or *—C(═O)—NH—,
* designates a point of attachment to the carbon atom in the backbone,
R¹⁴ is halogen, cyano, hydroxy, nitro, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom,
AL¹ and AL² are each independently an acid labile group, and
a is an integer of 0 to 4.

The polymer may further comprise repeat units having the formula (b3).

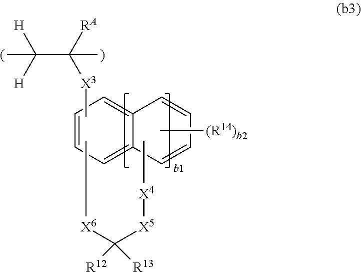

Herein b1 is 0 or 1, b2 is an integer of 0 to 3 when b1=0 and an integer of 0 to 5 when b1=1,
R^{A} is hydrogen, fluorine, methyl or trifluoromethyl,
X³ is a single bond, *—C(═O)—O— or *—C(═O)—NH—, * designates a point of attachment to the carbon atom in the backbone,
R¹² and R¹³ are each independently hydrogen or a C₁-C₂₀ hydrocarbyl group which may contain a heteroatom, R¹² and R¹³ may bond together to form a ring with the carbon atom to which they are attached,
R¹⁴ is halogen, hydroxy, cyano, nitro, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group, a plurality of R¹⁴ may bond together to form a ring with the carbon atoms on the aromatic ring to which they are attached, when b2 is 2 or 3,
X⁴ is a single bond, C₁-C₄ aliphatic hydrocarbylene group, carbonyl group, sulfonyl group or a group obtained by combining the foregoing,
X⁵ and X⁶ are each independently oxygen or sulfur, with the proviso that X⁴ and X⁶ are attached to adjacent carbon atoms on the aromatic ring.

The polymer may further comprise repeat units having the formula (c1).

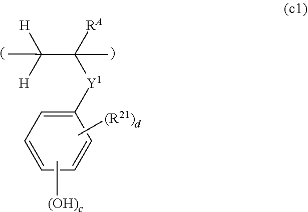

Herein R^{A} is hydrogen, fluorine, methyl or trifluoromethyl,
Y¹ is a single bond, *—C(═O)—O— or *—C(═O)—NH—, * designates a point of attachment to the carbon atom in the backbone,
R²¹ is halogen, nitro, cyano, carboxy, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom,
c is an integer of 1 to 4, d is an integer of 0 to 3, and c+d is from 1 to 5.

The polymer may further comprise repeat units having the formula (dl).

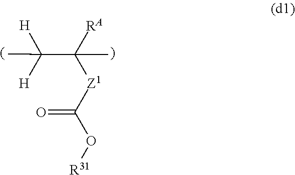

Herein R^{A} is hydrogen, fluorine, methyl or trifluoromethyl,
Z¹ is a single bond, phenylene group, naphthylene group, *—C(═O)—O—Z¹¹— or *—C(═O)—NH—Z¹¹—, the phenylene and naphthylene groups may be substituted with hydroxy, nitro, cyano, a C₁-C₁₀ alkoxy moiety which may contain a heteroatom, or halogen, * designates a point of attachment to the carbon atom in the backbone, Z¹¹ is a C₁-C₁₀ saturated hydrocarbylene group, phenylene group or naphthylene group, the saturated hydrocarbylene group may contain hydroxy, ether bond, ester bond or lactone ring,
R³¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring, sultone ring, and carboxylic anhydride (—C(═O)—O—C(═O)—).

In a still further aspect, the invention provides a chemically amplified resist composition comprising (A) a base polymer containing the polymer defined above.

The resist composition may further comprise (B) an organic solvent, (C) a quencher, (D) an acid generator, (E) a surfactant, and/or (F) a dissolution inhibitor.

In a still further aspect, the invention provides a pattern forming process comprising the steps of applying the chemically amplified resist composition defined above onto a substrate to form a resist film thereon, exposing the resist film to high-energy radiation, and developing the exposed resist film in a developer.

Typically, the high-energy radiation is ArF excimer laser of wavelength 193 nm, KrF excimer laser of wavelength 248 nm, EB or EUV of wavelength 3 to 15 nm.

### Advantageous Effects of Invention

A polymer comprising repeat units derived from an onium salt monomer consisting of a fluorosulfonic acid anion containing an aromatic ring structure substituted with a polymerizable group and iodine and a triarylsulfonium cation containing iodine and fluorine, the repeat units being capable of generating an acid upon light exposure, has good solvent solubility due to an improvement in lipophilicity by fluorine. In the triarylsulfonium cation containing iodine and fluorine, particularly in the EUV lithography of wavelength 13.5 nm, iodine which is highly absorptive to EUV generates secondary electrons upon exposure. The electron withdrawing effect of fluorine serves to lower the energy level of the lowest unoccupied molecular orbital (LUMO) of the frontier orbital theory so that the cation is more likely to accept the generated secondary electrons, whereby the decomposition of the cation is promoted and the acid is effectively generated. In the fluorosulfonic acid anion containing an aromatic ring structure substituted with a polymerizable group and iodine, on the other hand, the anion generates secondary electrons because the anion also contains iodine, whereby the decomposition of the cation is promoted. The synergy of these effects results in a higher sensitivity. Since iodine has a large atomic weight and the generated acid is bound to the polymer backbone, the advantage of reduced acid diffusion is obtained. This prevents any lowering of resolution due to blur by acid diffusion, contributing an improvement in LWR or CDU. The aromatic ring in the repeat unit serves as an effective etch resistant group, which is advantageous in forming small-size patterns.

### DETAILED DESCRIPTION OF THE INVENTION

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, Me stands for methyl, Ac for acetyl. Both the broken line ( - - - ) and the asterisk (*) designate a point of attachment or valence bond. As used herein, the term “fluorinated” refers to a fluorine-substituted or fluorine-containing compound or group, and “iodized” refers to an iodine-substituted or iodine-containing compound or group. The terms “group” and “moiety” are interchangeable.

The abbreviations and acronyms have the following meaning.
EB: electron beam
EUV: extreme ultraviolet
Mw: weight average molecular weight
Mn: number average molecular weight
Mw/Mn: molecular weight distribution or dispersity
GPC: gel permeation chromatography
PEB: post-exposure bake
PAG: photoacid generator
LWR: line width roughness
EL: exposure latitude
DOF: depth of focus
CDU: critical dimension uniformity

### [Onium Salt Monomer]

One embodiment of the invention is an onium salt monomer containing an anion having the formula (a1).

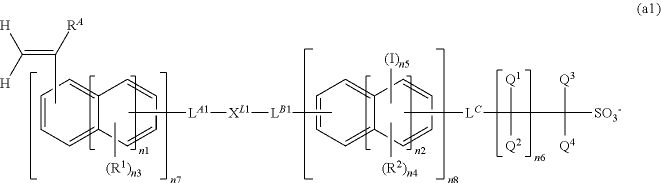

In formula (a1), n1 is 0 or 1. The relevant structure is a benzene ring in case of n1=0 and a naphthalene ring in case of n1=1. From the aspect of solvent solubility, the benzene ring corresponding to n1=0 is preferred. The subscript n2 is 0 or 1. The relevant structure is a benzene ring in case of n2=0 and a naphthalene ring in case of n2=1. From the aspect of solvent solubility, the benzene ring corresponding to n2=0 is preferred.

In formula (a1), n3 is an integer of 0 to 4. It is preferred from the aspect of reactant availability that n3 be 0, 1 or 2, more preferably 0 or 1. The subscript n4 is an integer of 0 to 4, preferably 0, 1, 2 or 3, more preferably 0, 1 or 2, even more preferably 0 or 1.

In formula (a1), n5 is an integer of 1 to 6. As the number of iodine atoms in the anion structure is more, the absorption of EUV becomes higher, but solvent solubility becomes poorer. There is left concern that the compound precipitates in the resist composition. Thus n5 is preferably 1, 2 or 3, more preferably 1 or 2. It is noted that n4+n5 is from 1 to 4 when n2=0, and n4+n5 is from 1 to 6 when n2=1.

In formula (a1), n6 is an integer of 0 to 4, preferably 0, 1, 2 or 3, most preferably 1; n7 is 0 or 1; and n8 is 0 or 1. Notably, n7 and n8 are not equal to 0 at the same time.

In formula (a1), R^{A} is hydrogen, fluorine, methyl or trifluoromethyl, preferably hydrogen or methyl, most preferably hydrogen.

In formula (a1), iodine on the aromatic ring in the anion is preferably bonded at the ortho position relative to the carbon atom to which LB1 is attached. Since iodine has a large atomic radius, the rotation of the bond axis between the aromatic ring to which the polymerizable group is attached and the aromatic ring to which iodine is attached is restrained. The polymer thus becomes more robust.

In formula (a1), R¹ is halogen, nitro, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom. Suitable halogen atoms include fluorine, chlorine, bromine and iodine, with fluorine and iodine being preferred. The hydrocarbyl group and hydrocarbyl moiety in the hydrocarbyloxy and hydrocarbylthio groups may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cylopropylmethyl, 4-methylcyclohexyl, cyclohexylmethyl, norbornyl, and adamantyl; C₂-C₂₀ alkenyl groups such as vinyl, allyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl, and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. A plurality of R¹ may be identical or different when n3 is 2, 3 or 4 and n7 is 1.

When n3 is 2, 3 or 4 and n7 is 1, a plurality of R¹ may bond together to form a ring with the carbon atoms on the aromatic ring to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety. Notably, when n8=0, n7 is 1 and n3 is 1, 2, 3 or 4, and at least one of R¹ is iodine.

In formula (a1), R² is halogen exclusive of iodine, nitro, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom. Exemplary of the halogen exclusive of iodine are fluorine, chlorine and bromine. The hydrocarbyl group and hydrocarbyl moiety in the hydrocarbyloxy and hydrocarbylthio groups may be saturated or unsaturated and straight, branched or cyclic. Examples thereof are as exemplified above for the hydrocarbyl group R¹, but not limited thereto. A plurality of R² may be identical or different when n4 is 2, 3 or 4 and n8 is 1.

When n4 is 2, 3 or 4 and n8 is 1, a plurality of R² may bond together to form a ring with the carbon atoms to which they are attached. Of the rings, 5 to 8-membered rings are preferred.

In formula (a1), L^{A1}, L^{B1} and L^{C} are each independently a single bond, ether bond, ester bond, sulfonate ester bond, amide bond, sulfonamide bond, carbonate bond or carbamate bond. L^{A1} is preferably a single bond, ether bond, ester bond or sulfonate ester bond, more preferably ester bond or sulfonate ester bond. L^{B1} is preferably a single bond, ether bond or ester bond, more preferably a single bond. L^{C} is preferably a single bond, ether bond, ester bond or sulfonate ester bond, more preferably ether bond or ester bond.

In formula (a1), X^{L1} is a single bond or a C₁-C₄₀ hydrocarbylene group which may contain a heteroatom. The hydrocarbylene group may be straight, branched or cyclic. Examples thereof include alkanediyl, cyclic saturated hydrocarbylene and arylene groups. Suitable heteroatoms include oxygen, nitrogen, and sulfur.

Examples of the optionally heteroatom-containing C₁-C₄₀ hydrocarbylene group X^{L1} are shown below, but not limited thereto. Herein * each designates a point of attachment to L^{A1} or L^{B1}.

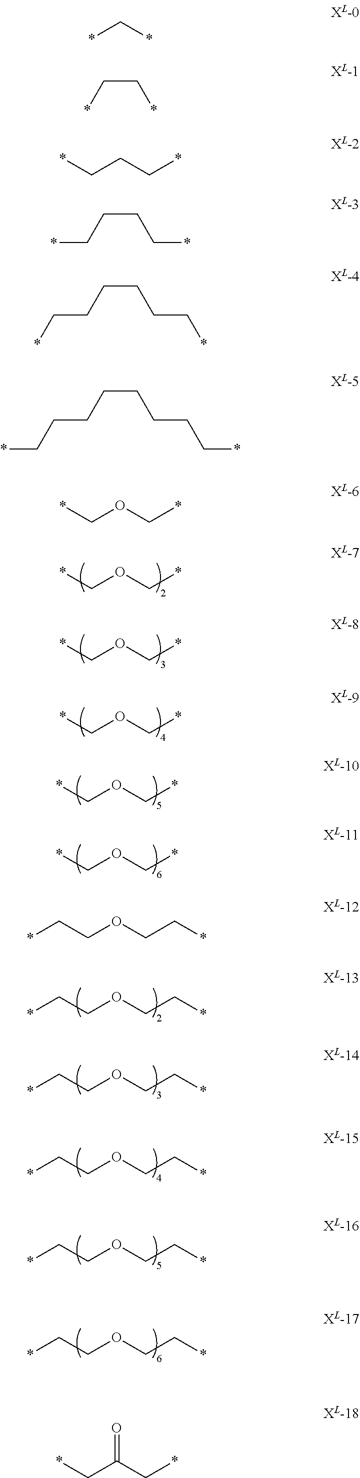

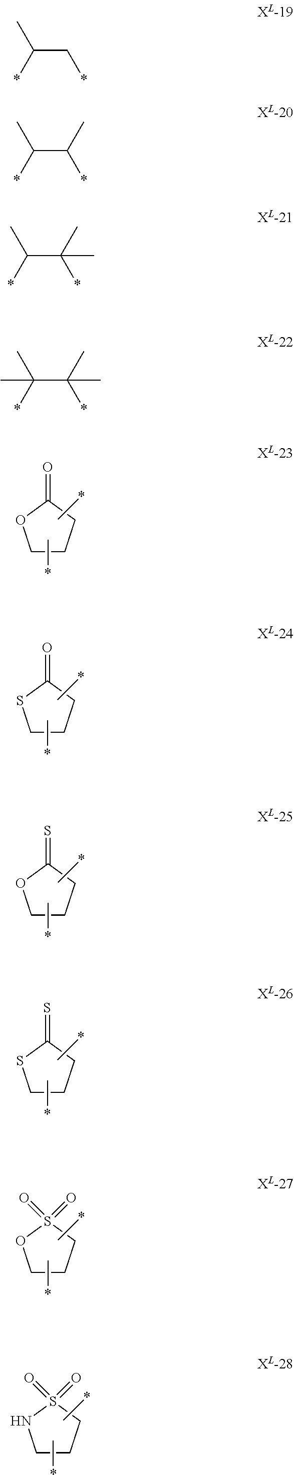

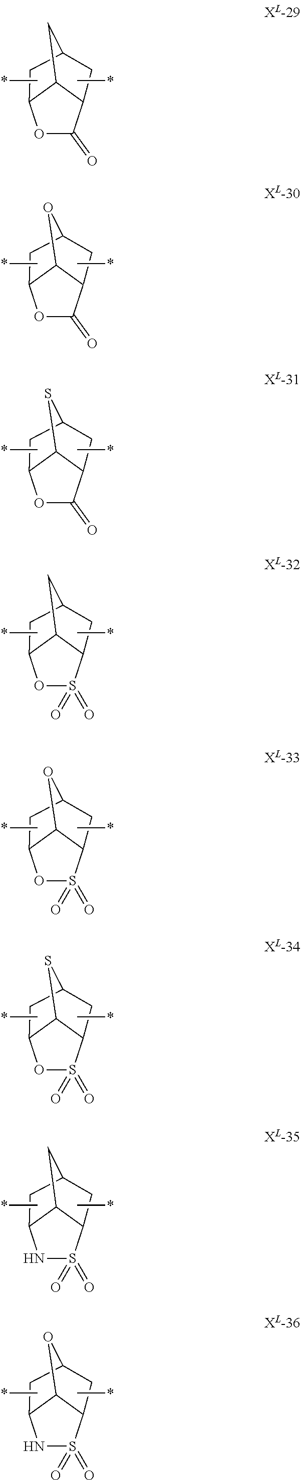

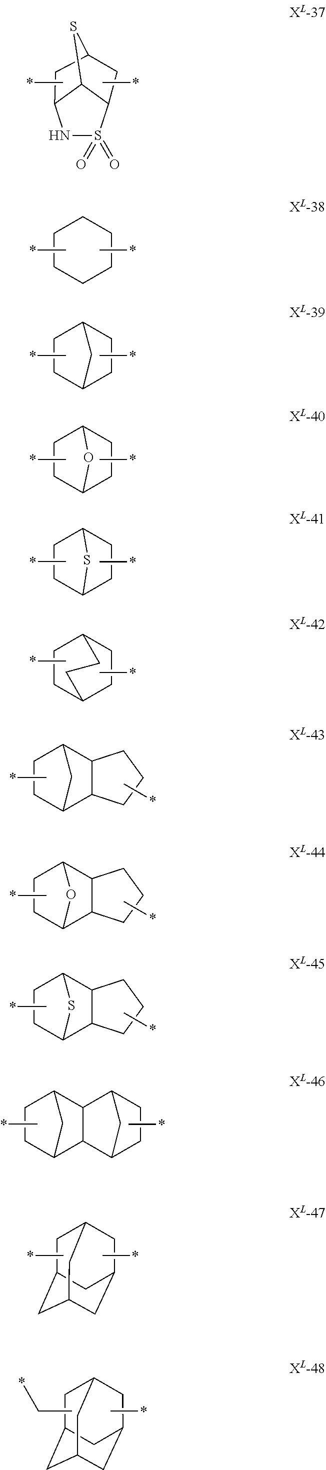

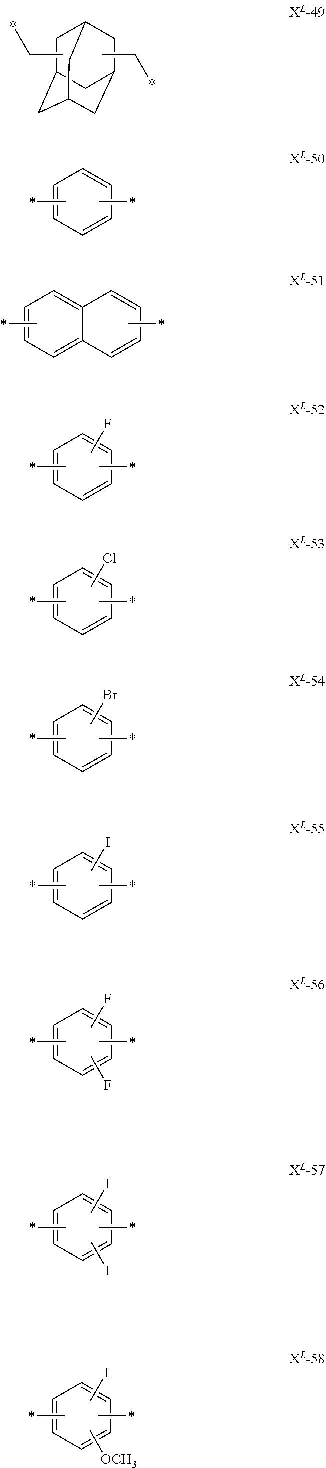

Of these, X^{L}-0 to X^{L}-22, X^{L}-29 to X^{L}-34, and X^{L}-47 to X^{L}-58 are preferred.

In formula (a1), Q¹ and Q² are each independently hydrogen, fluorine or a C₁-C₆ fluorinated saturated hydrocarbyl group. Typical of the fluorinated saturated hydrocarbyl group is trifluoromethyl.

In formula (a1), Q³ and Q⁴ are each independently fluorine or a C₁-C₆ fluorinated saturated hydrocarbyl group. Typical of the fluorinated saturated hydrocarbyl group is trifluoromethyl. More preferably, Q³ and Q⁴ are both fluorine.

Examples of the partial structure: —[C(Q¹)(Q²)]ₙ₆-C(Q³)(Q⁴)-SO₃— are shown below, but not limited thereto. Herein * designates a point of attachment to L^{C}.

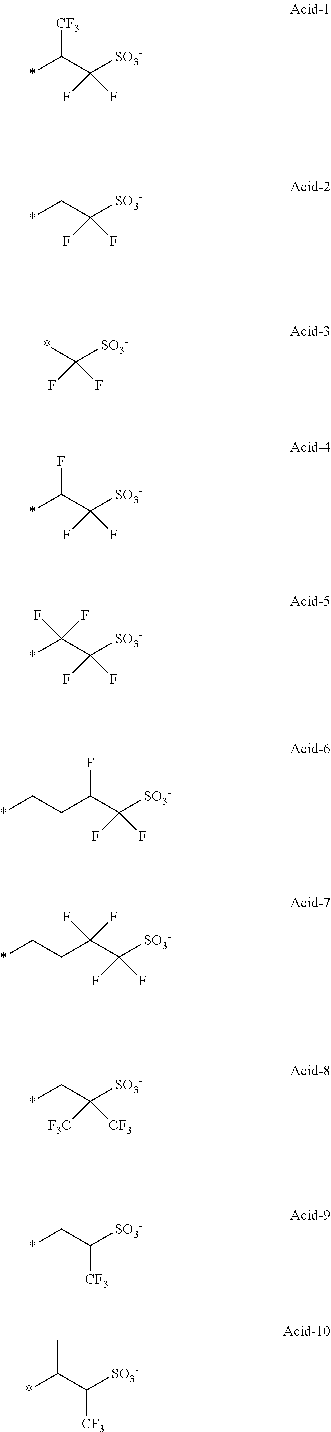

Of these, Acid-1 to Acid-7 are preferred, with Acid-1 to Acid-3, Acid-6 and Acid-7 being more preferred.

Of the anion structures having formula (a1), a structure having the formula (a1-1) is preferred.

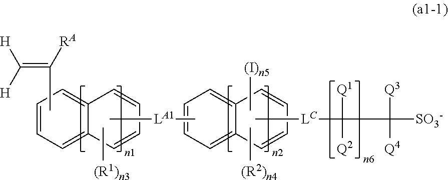

Herein n1 to n6, R^{A}, R¹, R², L^{A1} L^{C}, Q¹ to Q⁴ are as defined above.

Of the anion structures having formula (a1-1), a structure having the formula (a1-2) is preferred.

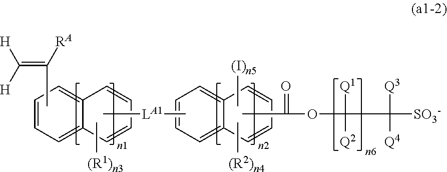

Herein n1 to n6, R^{A}, R¹, R², L^{A1}, Q¹ to Q⁴ are as defined above.

Examples of the anion having formula (a1) are shown below, but not limited thereto. Herein R^{A}, Q¹, R², n4 and n5 are as defined above. The positions of attachment of substituent groups on the aromatic ring are interchangeable.

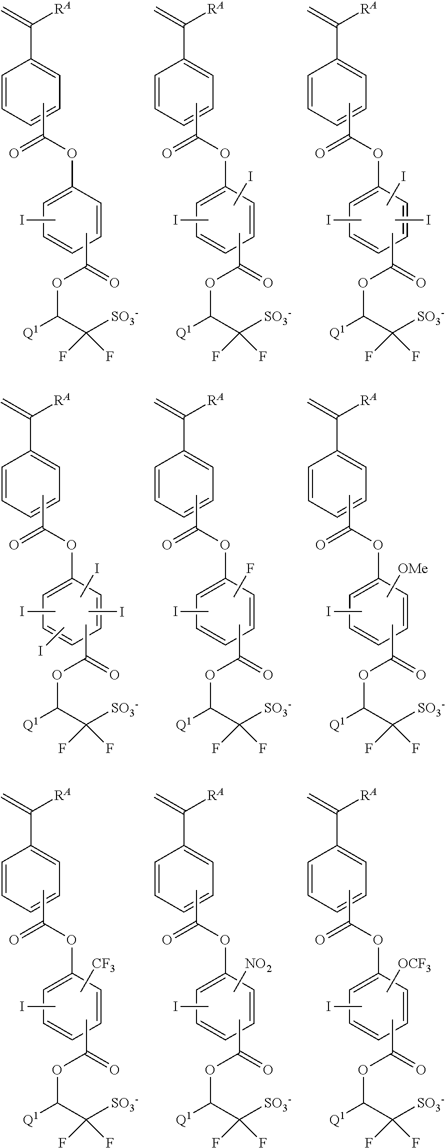

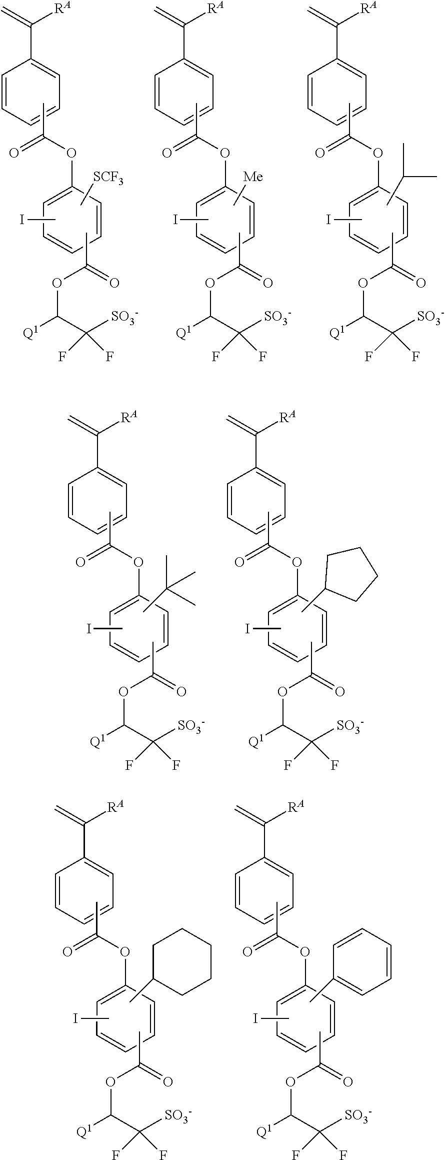

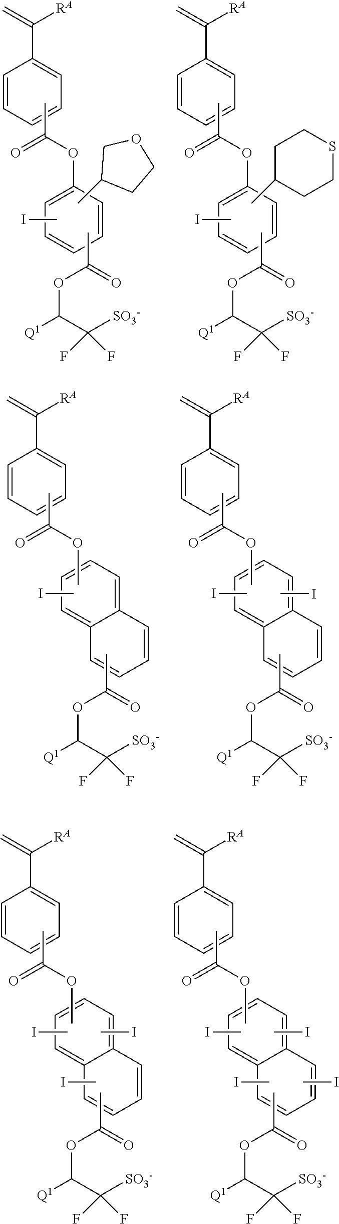

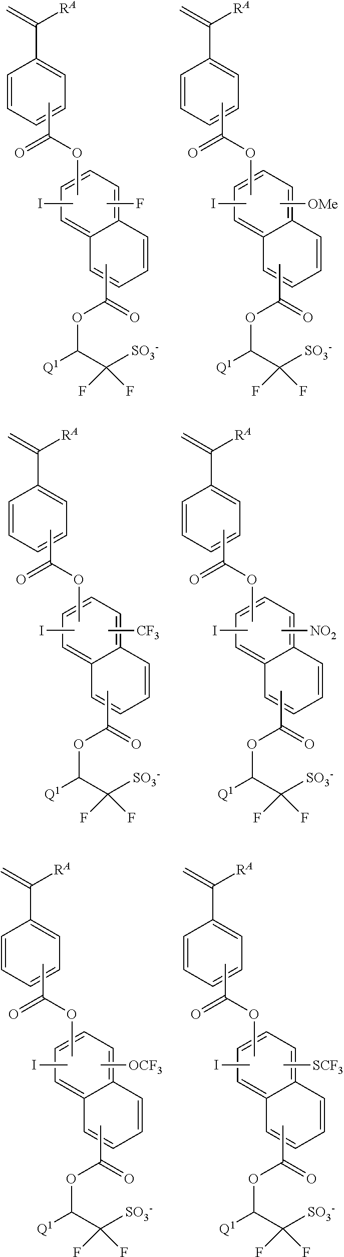

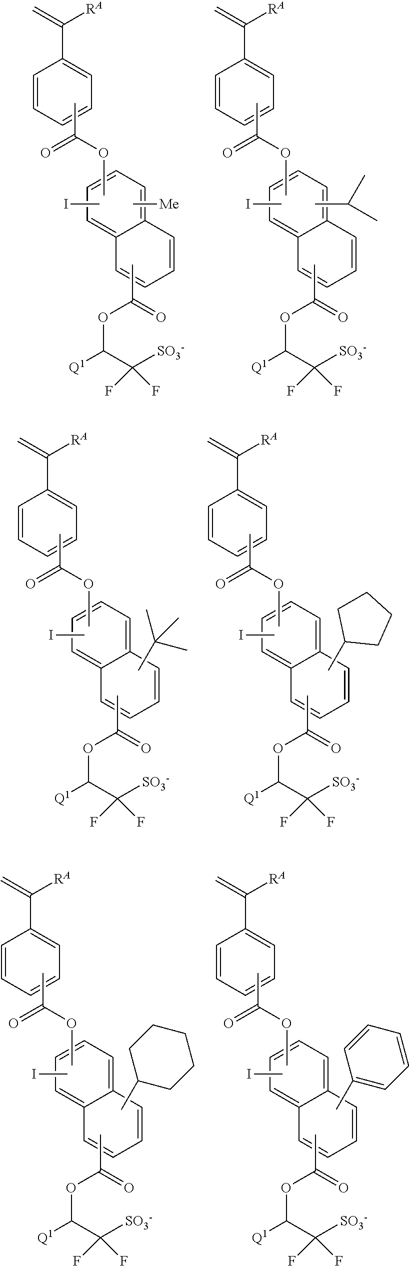

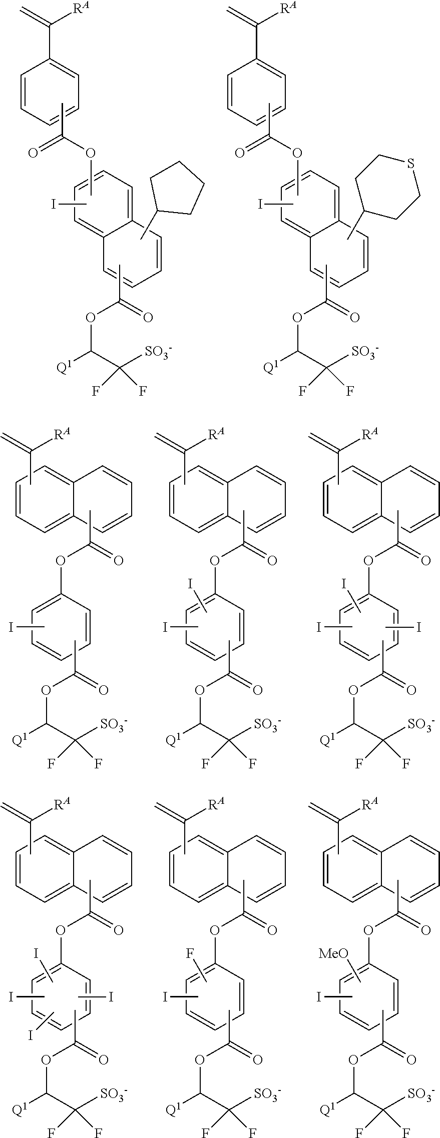

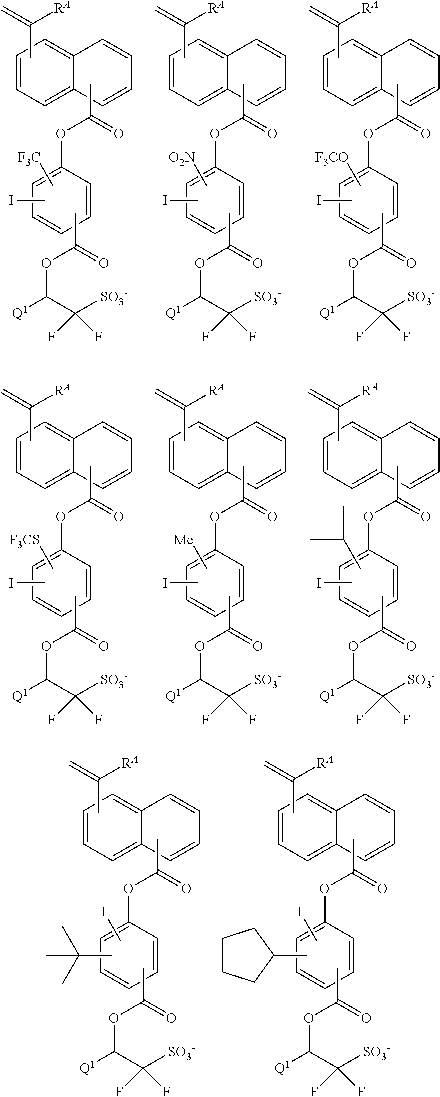

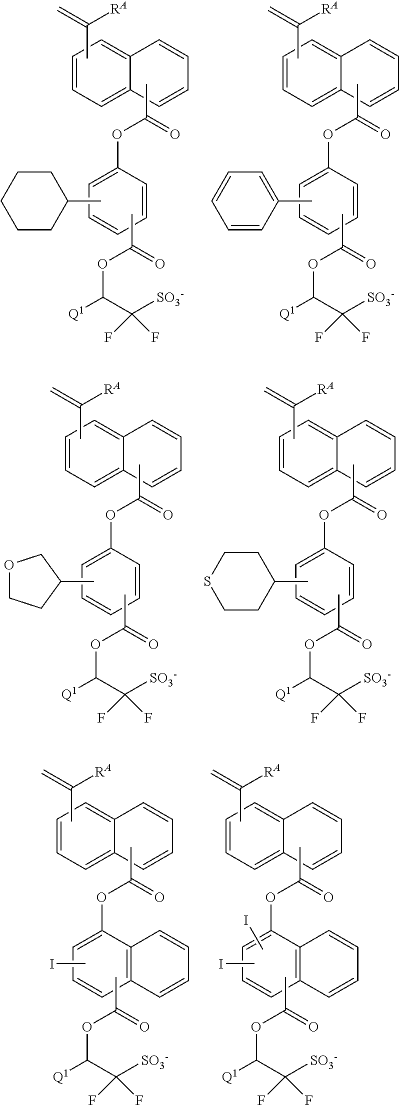

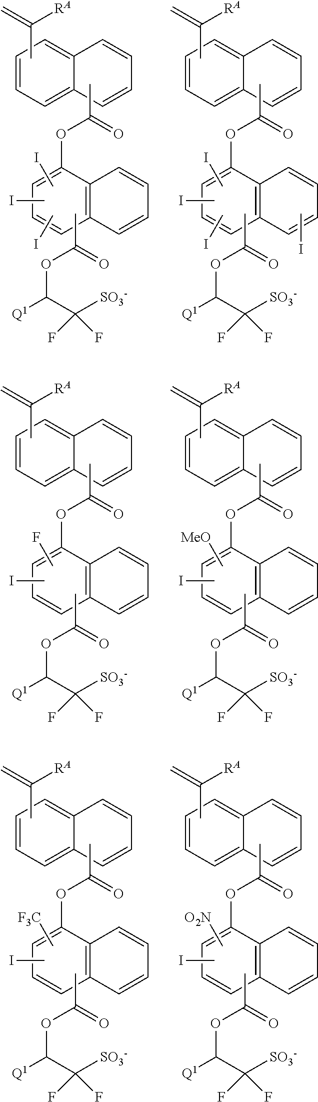

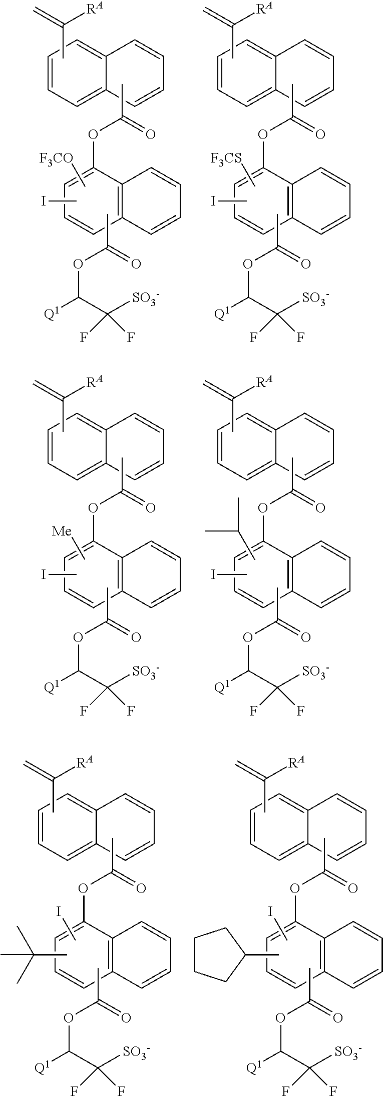

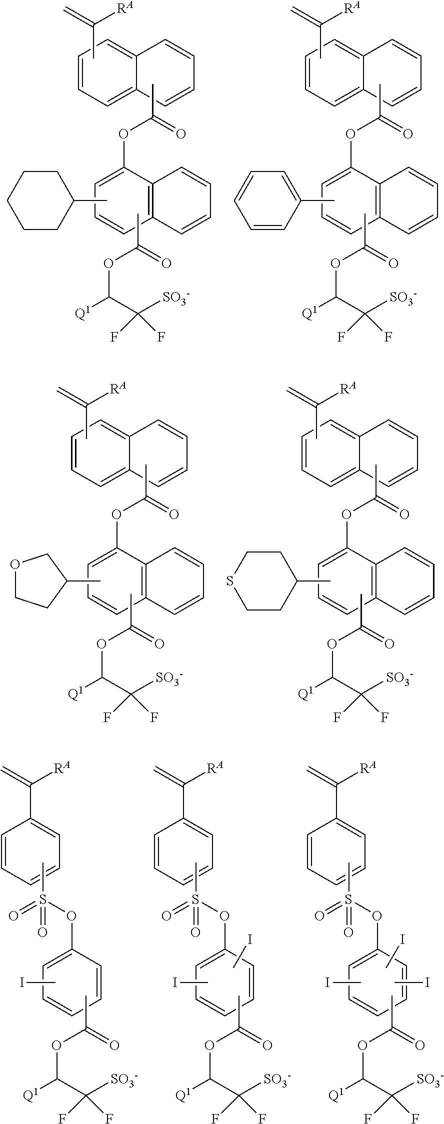

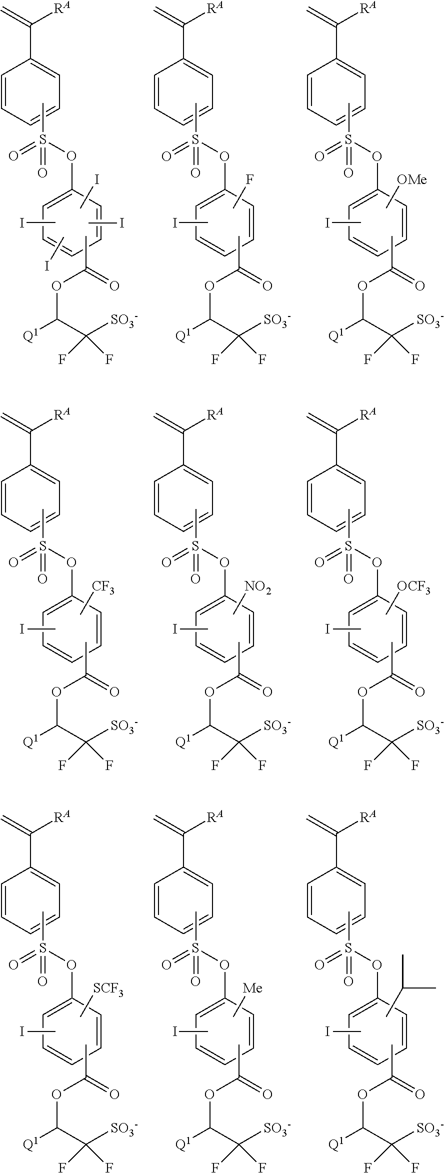

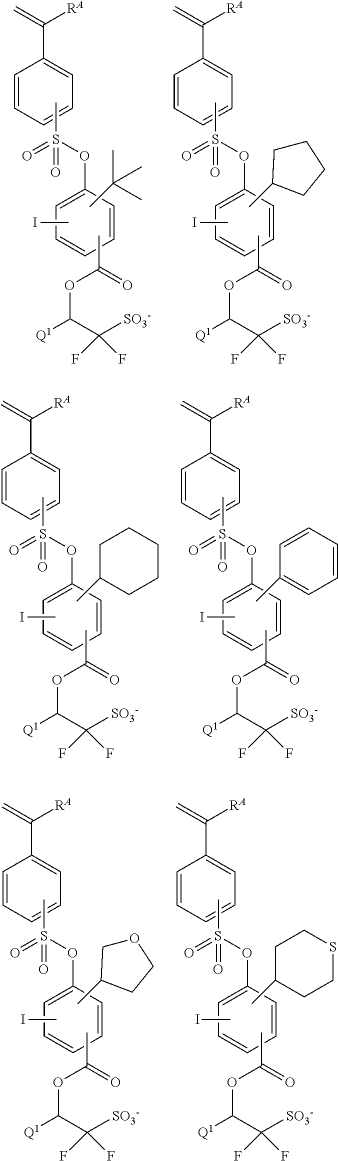

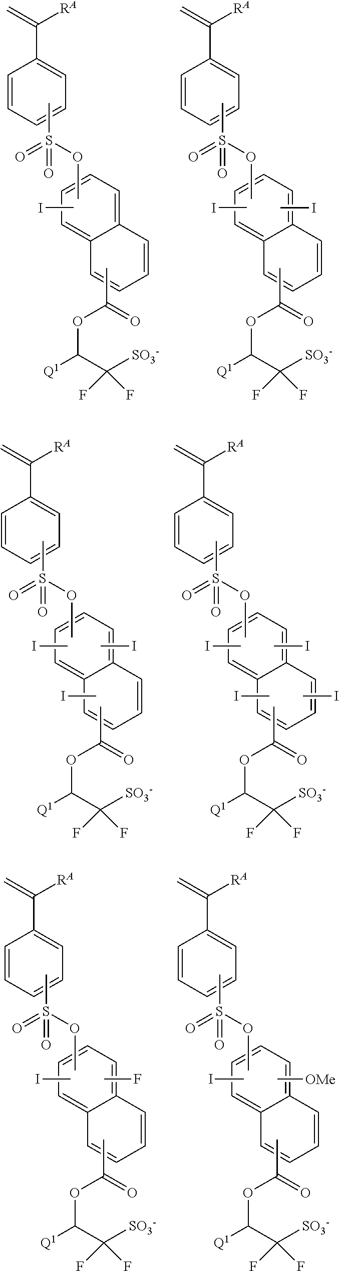

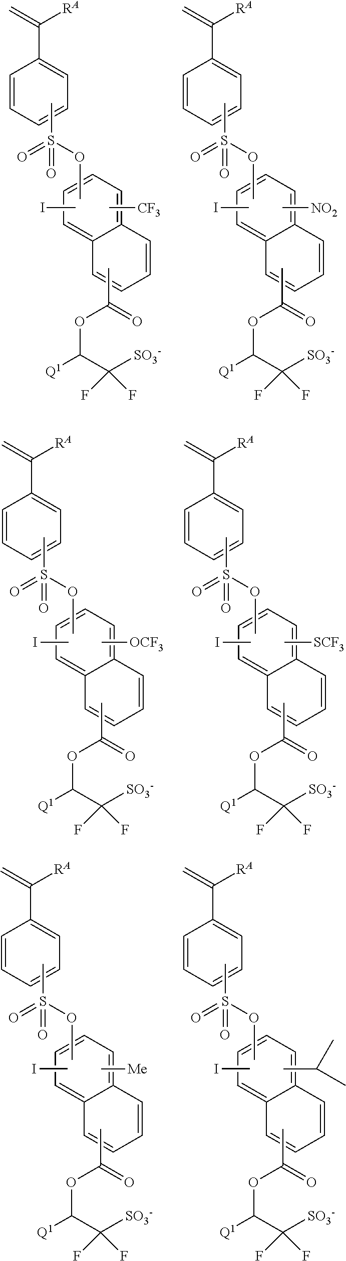

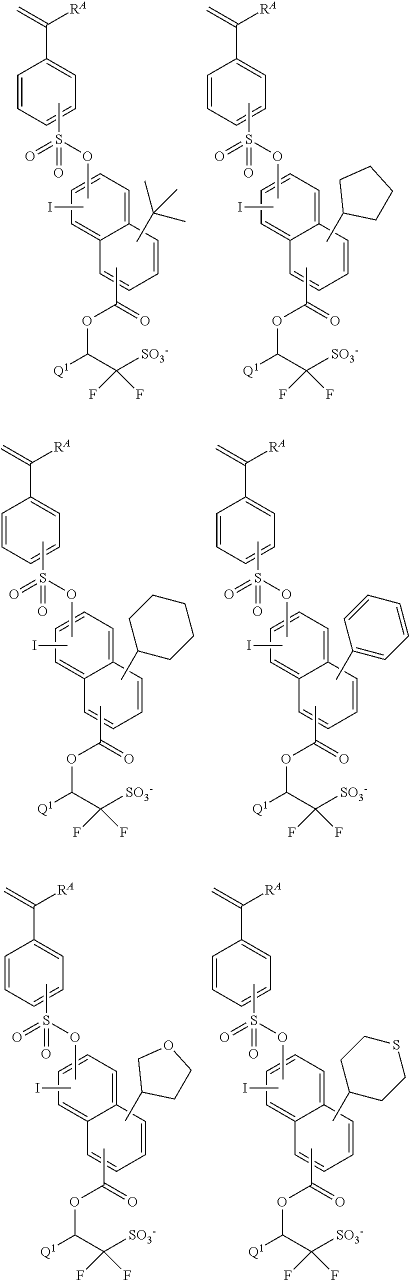

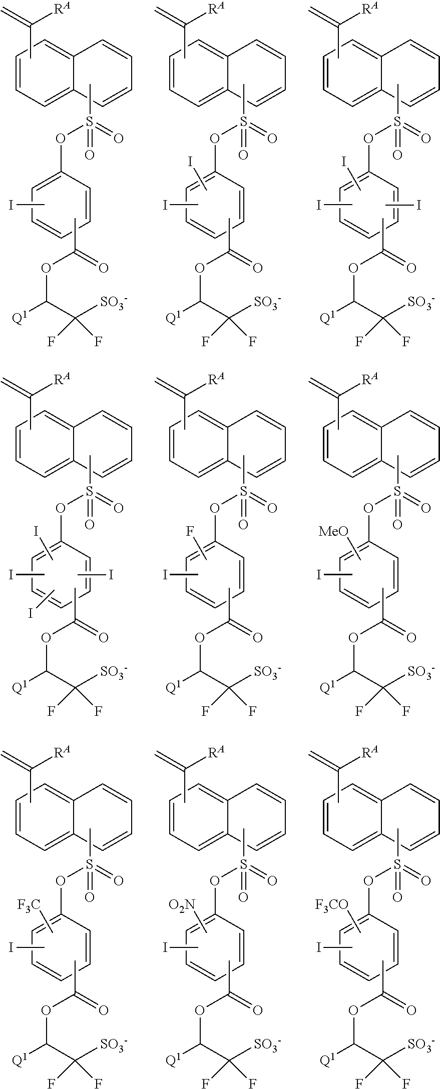

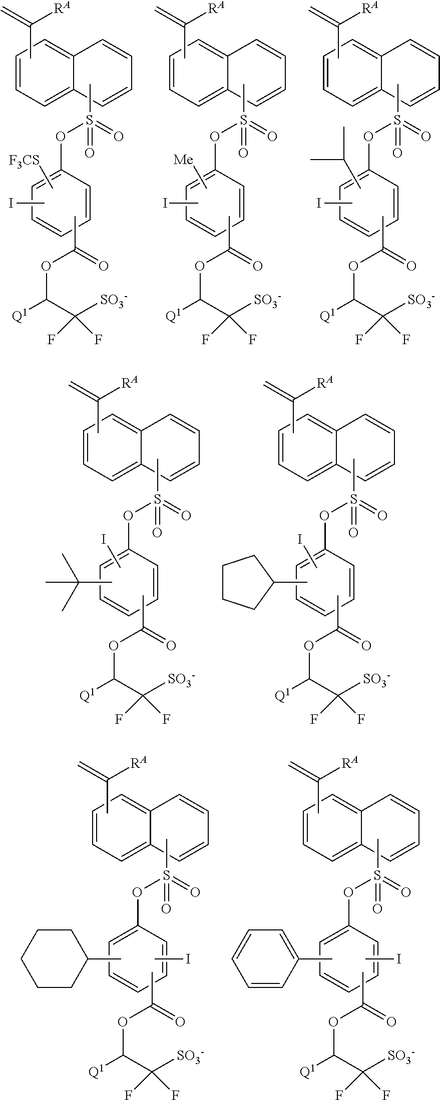

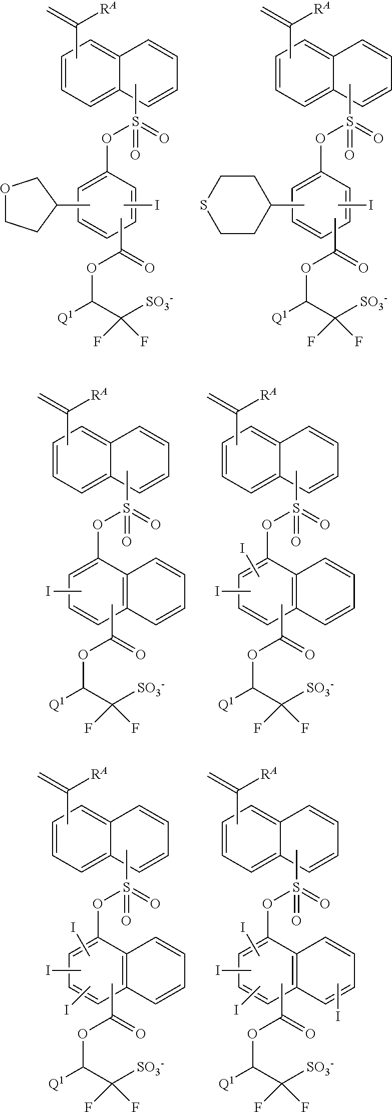

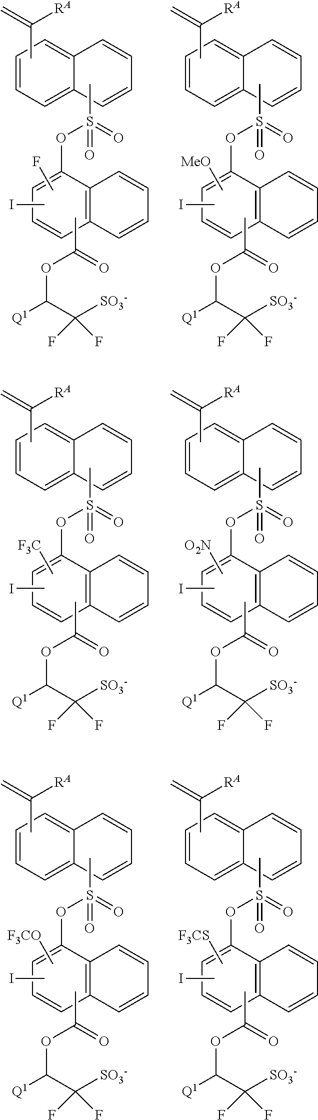

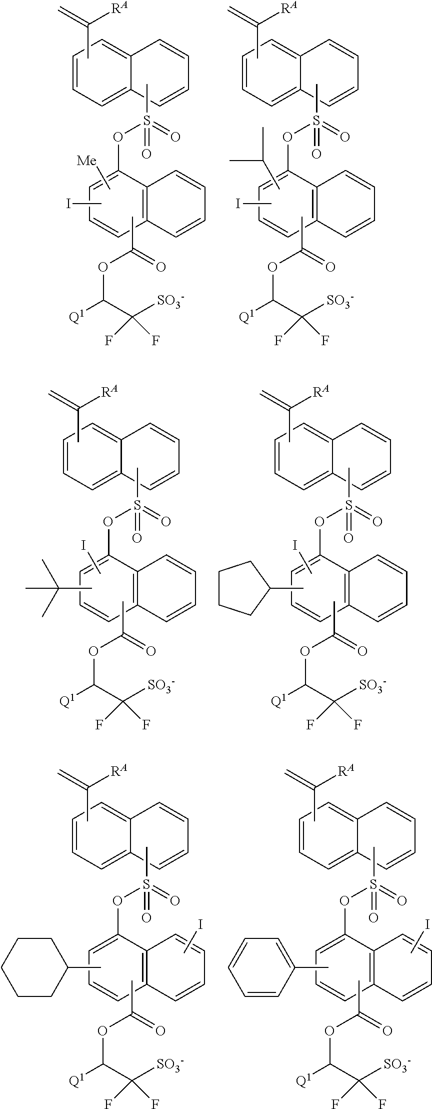

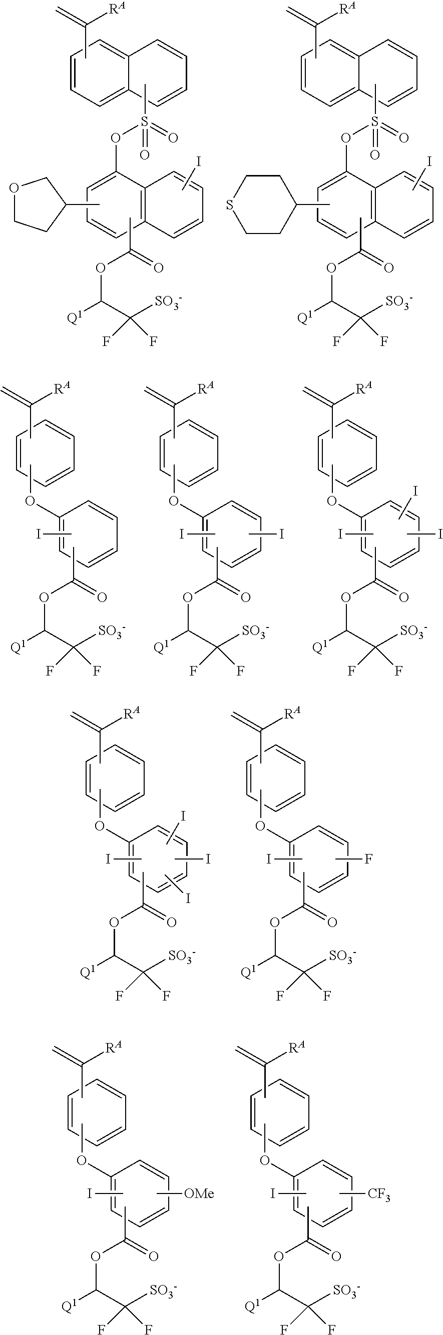

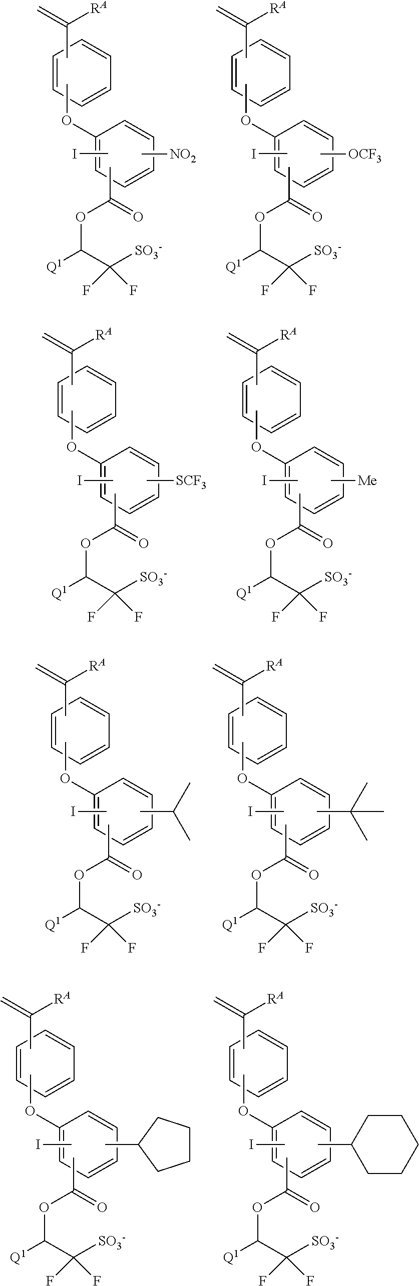

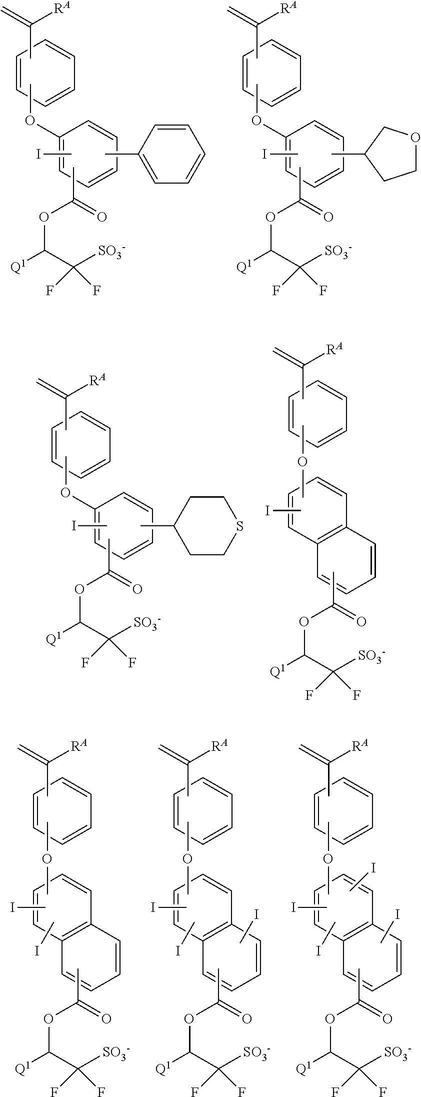

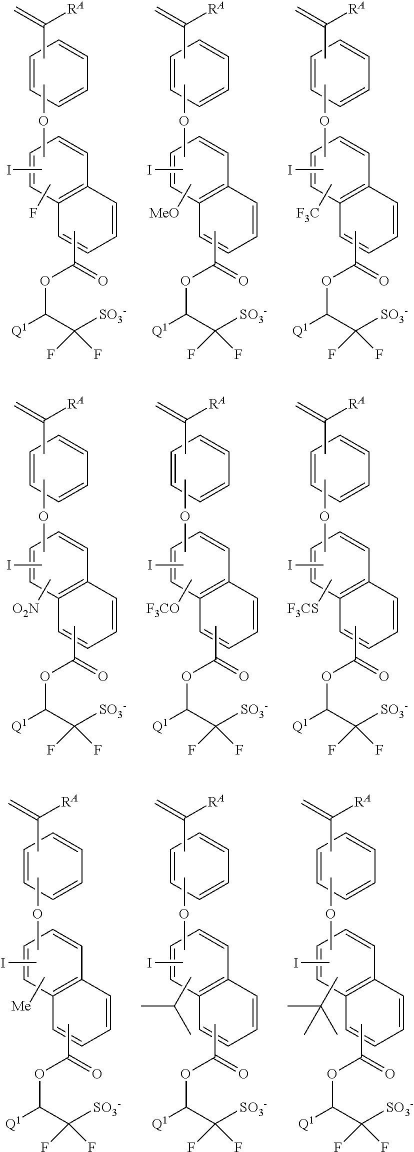

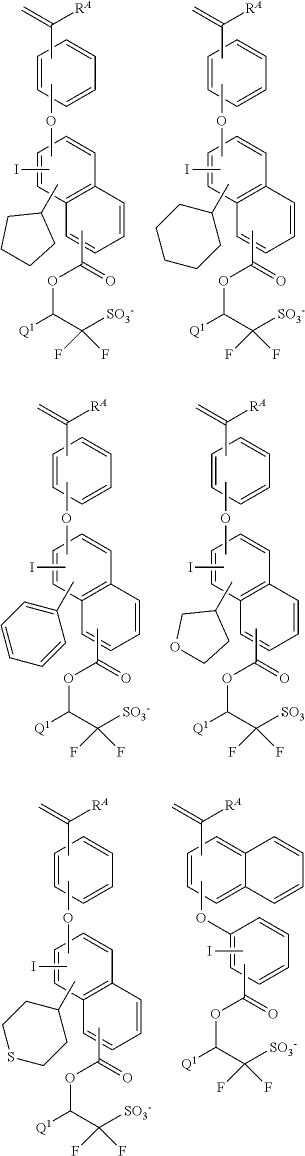

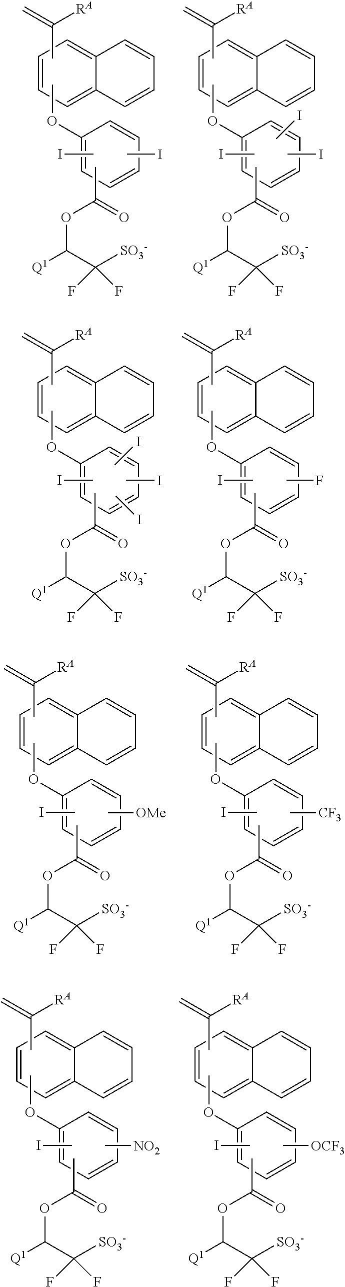

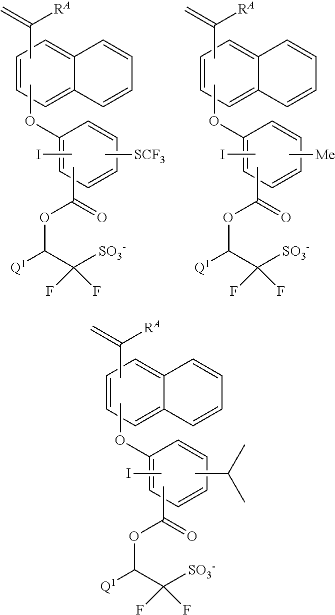

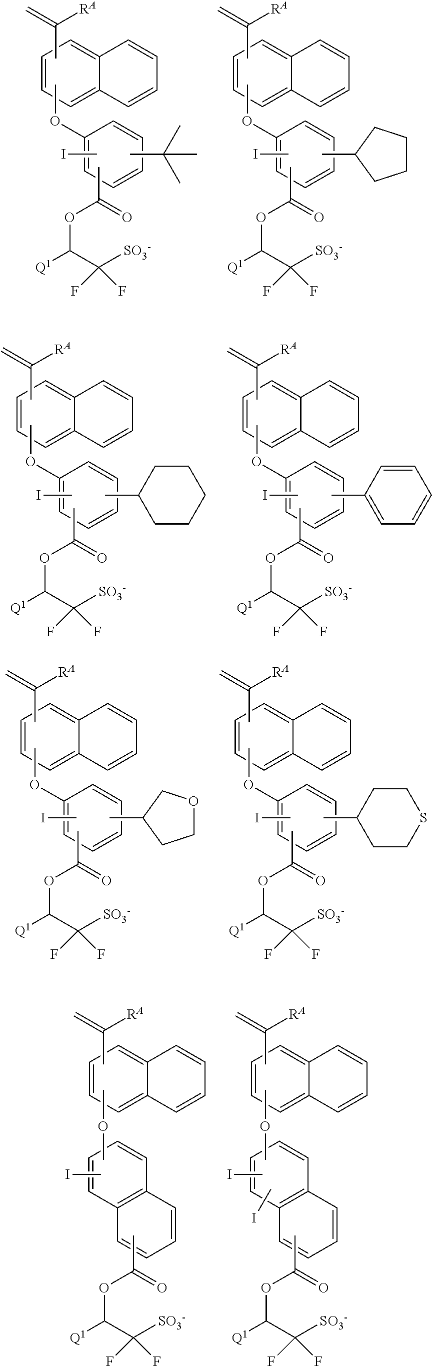

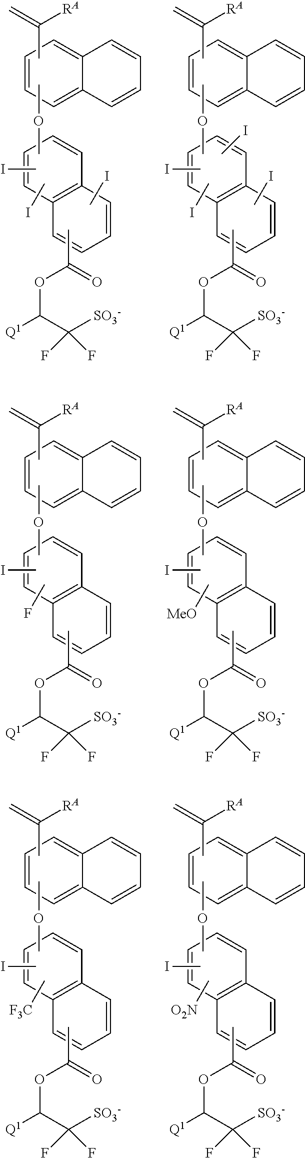

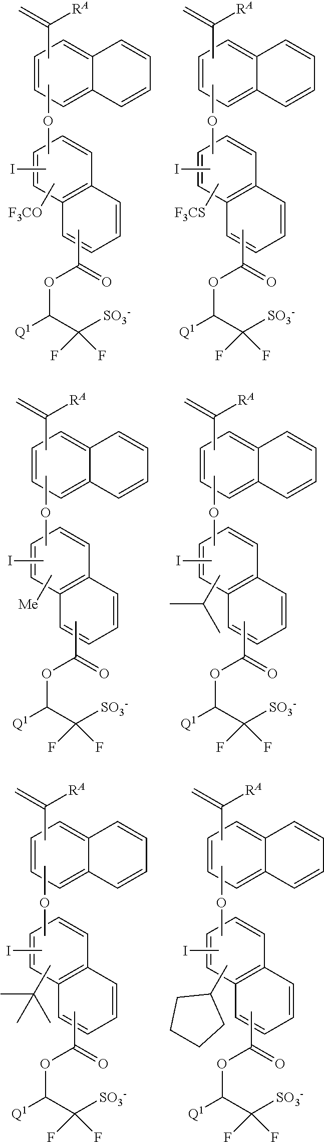

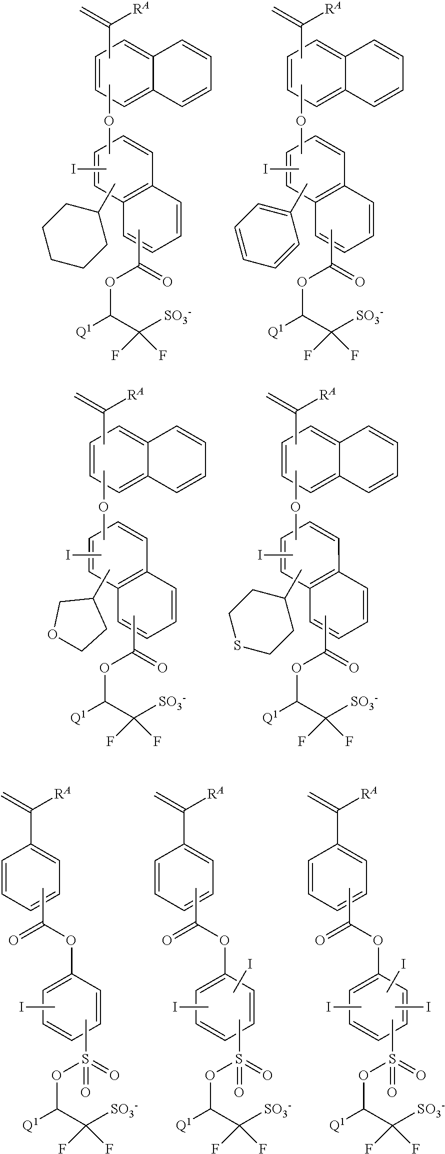

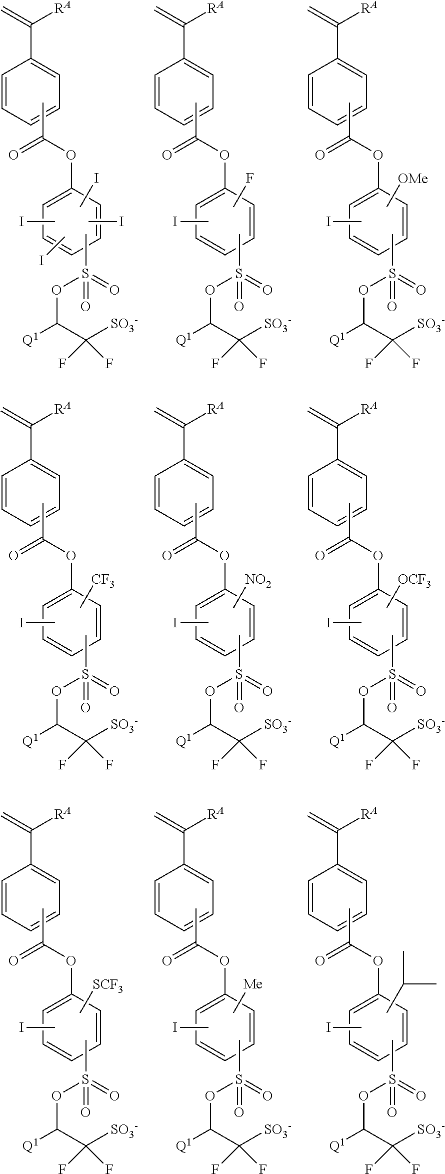

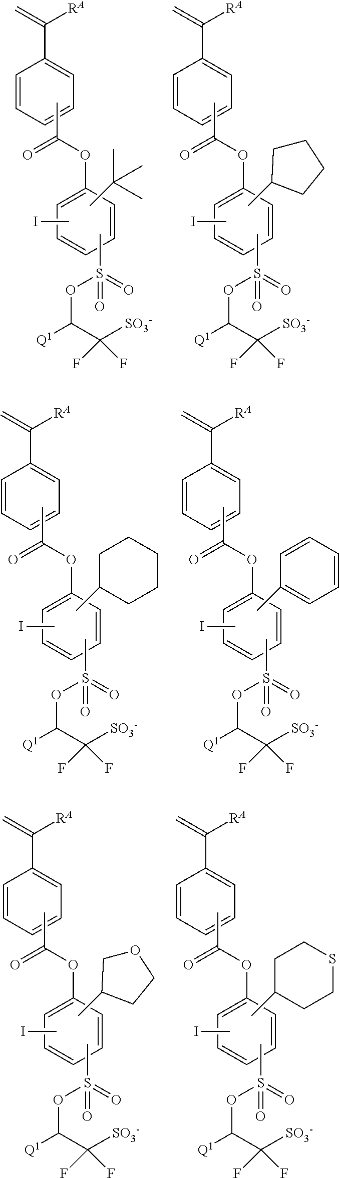

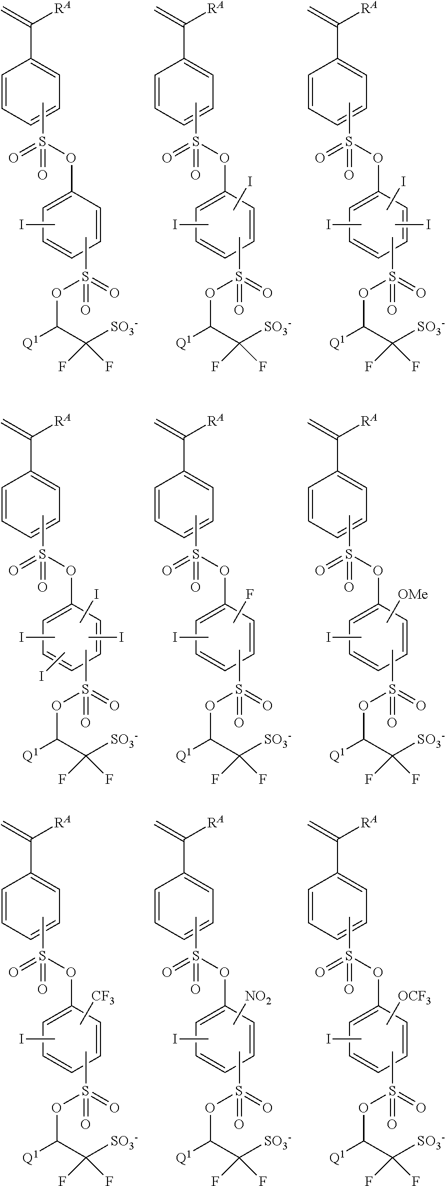

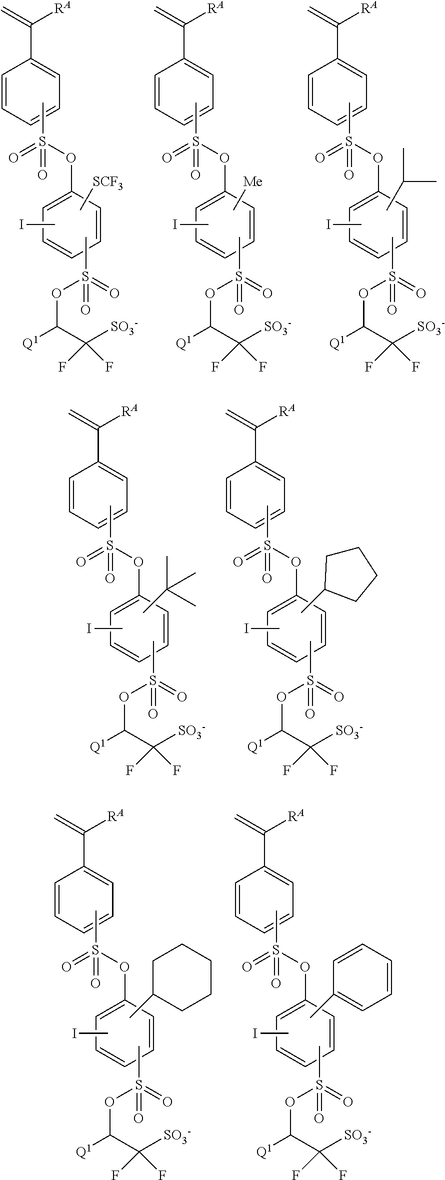

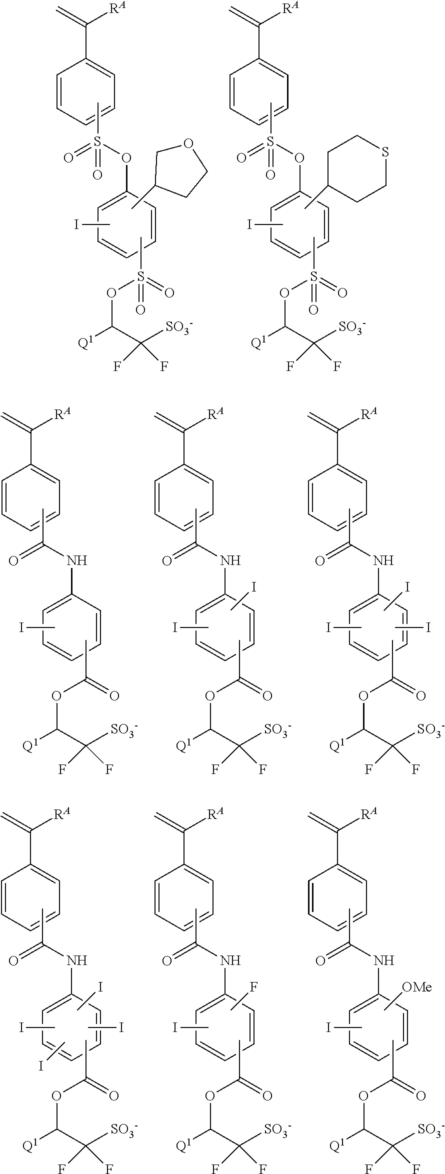

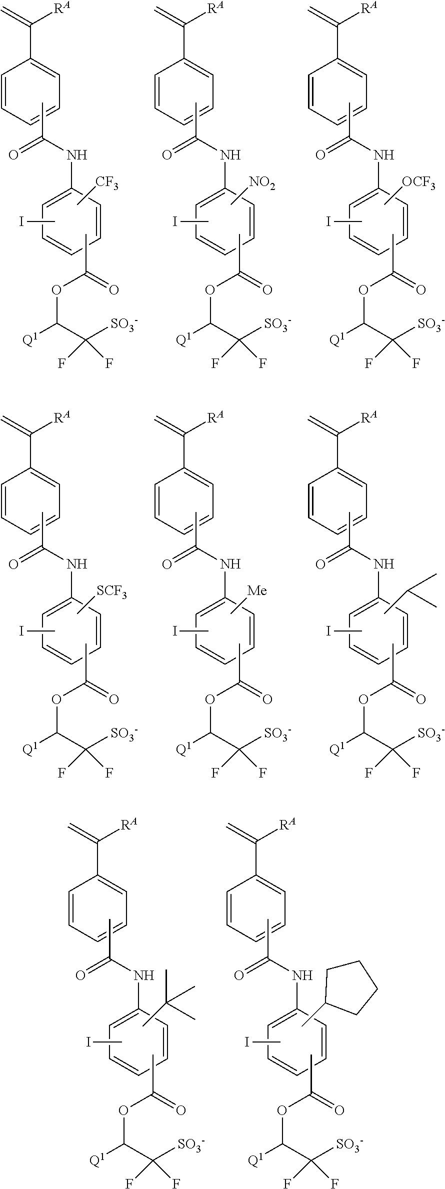

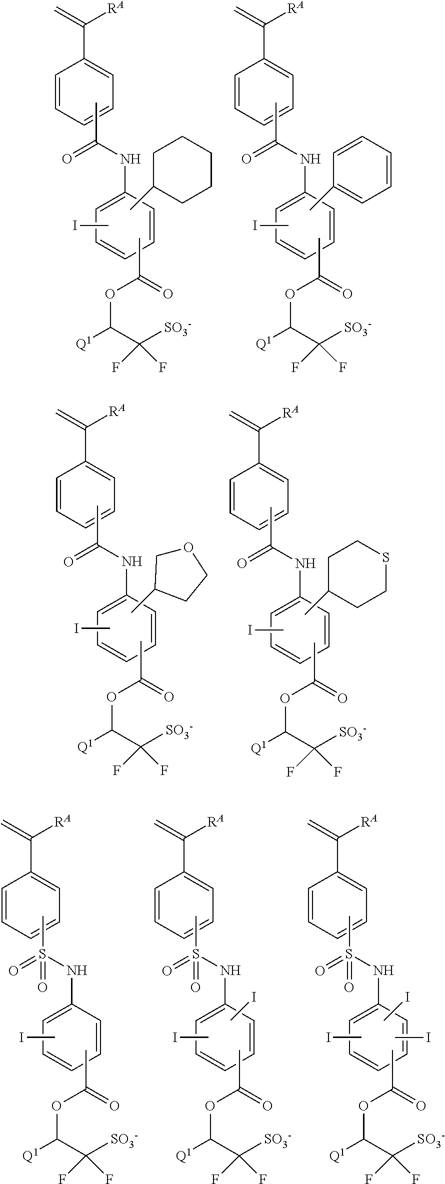

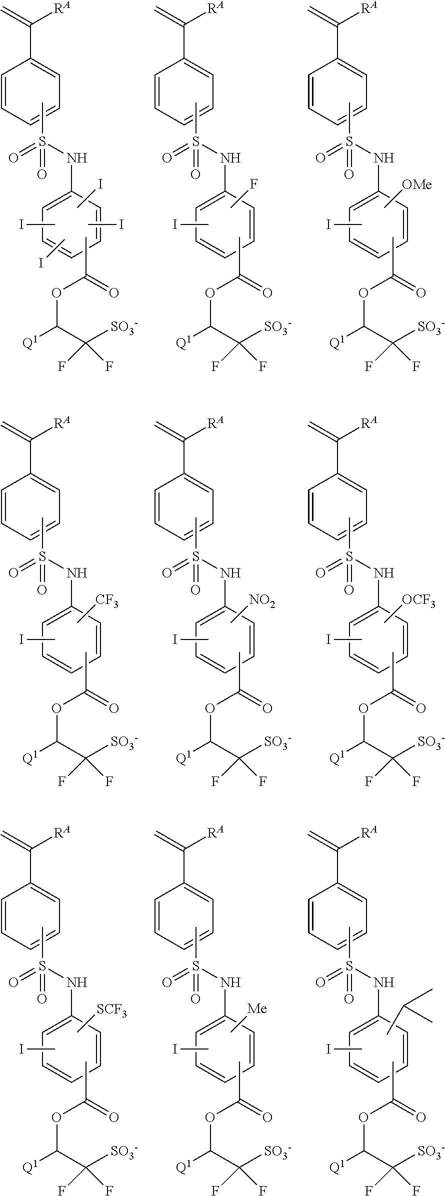

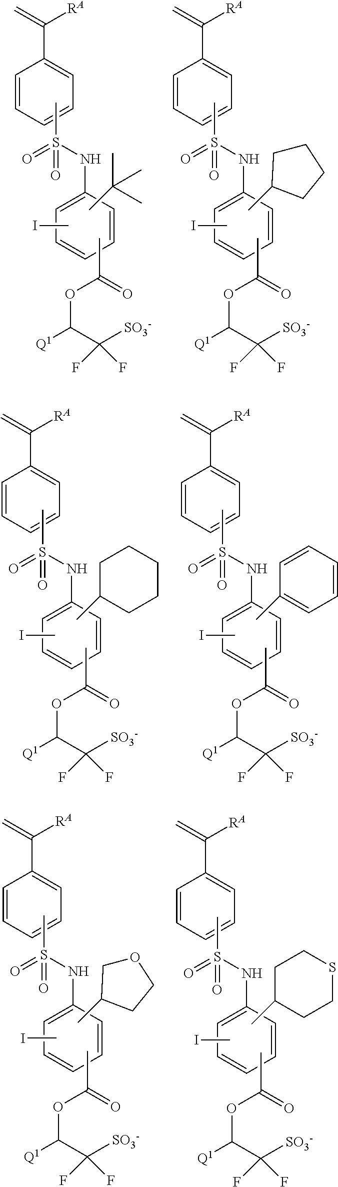

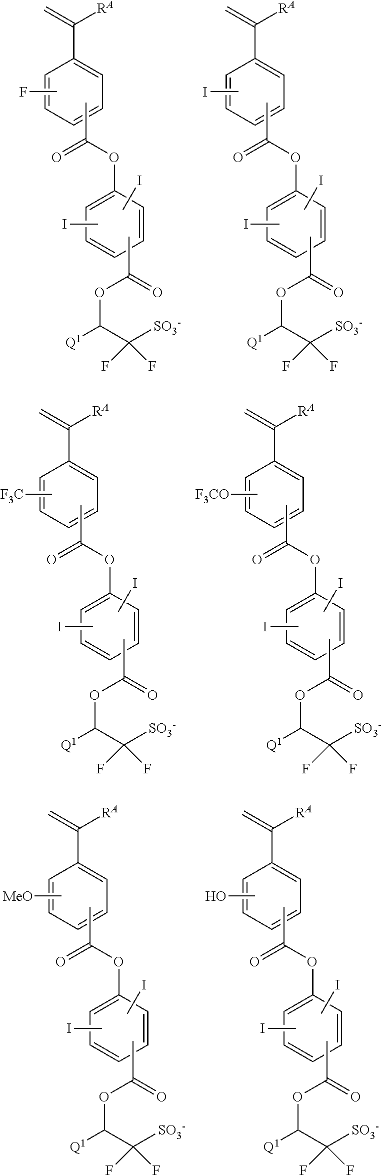

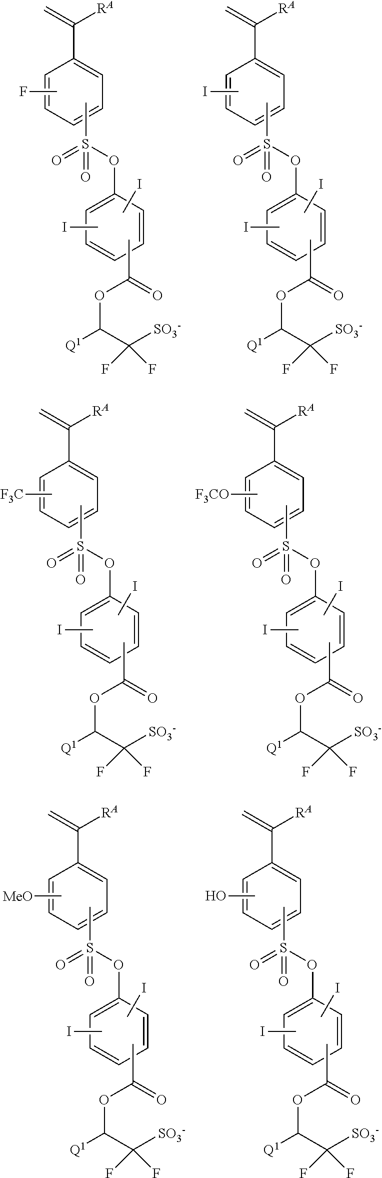

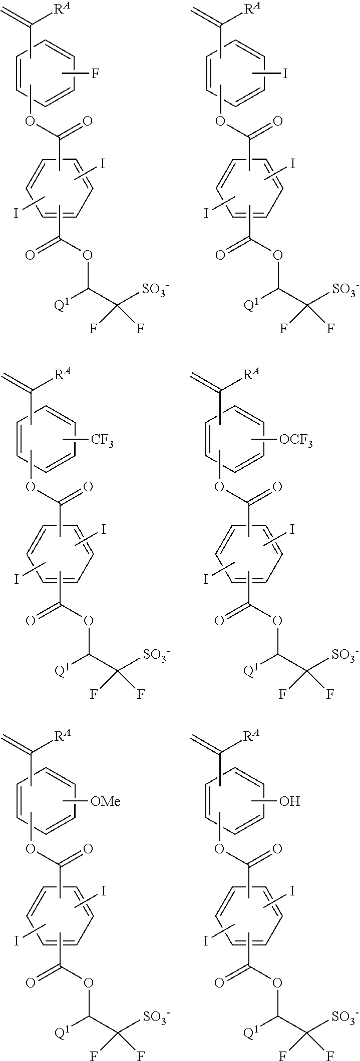

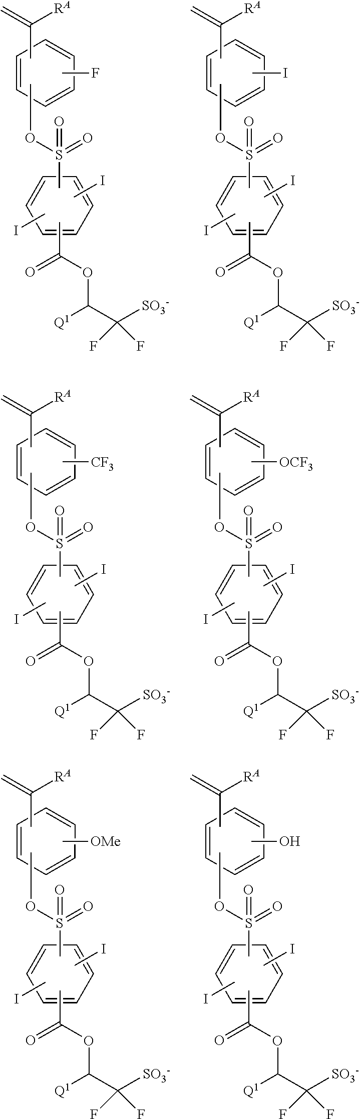

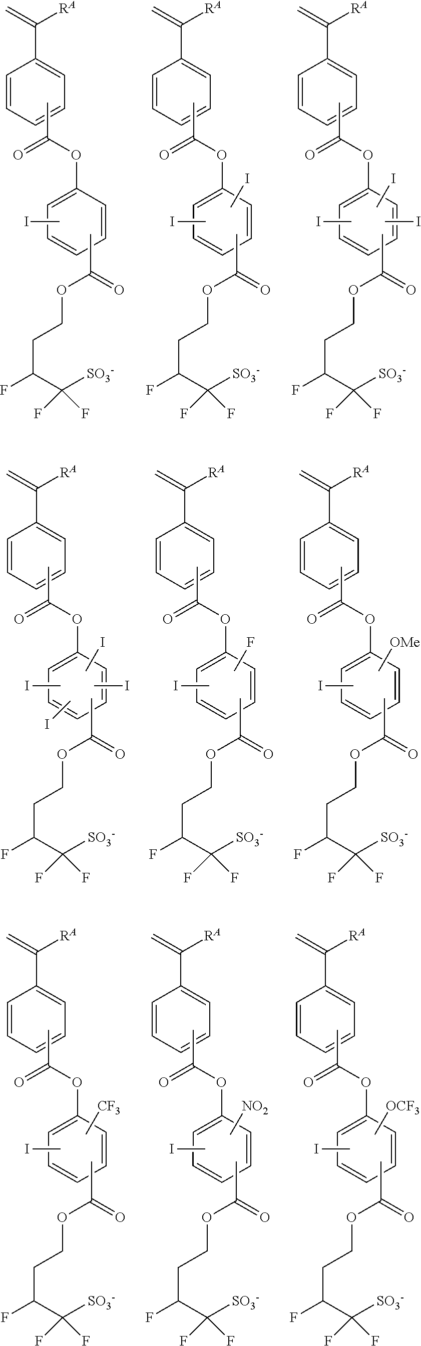

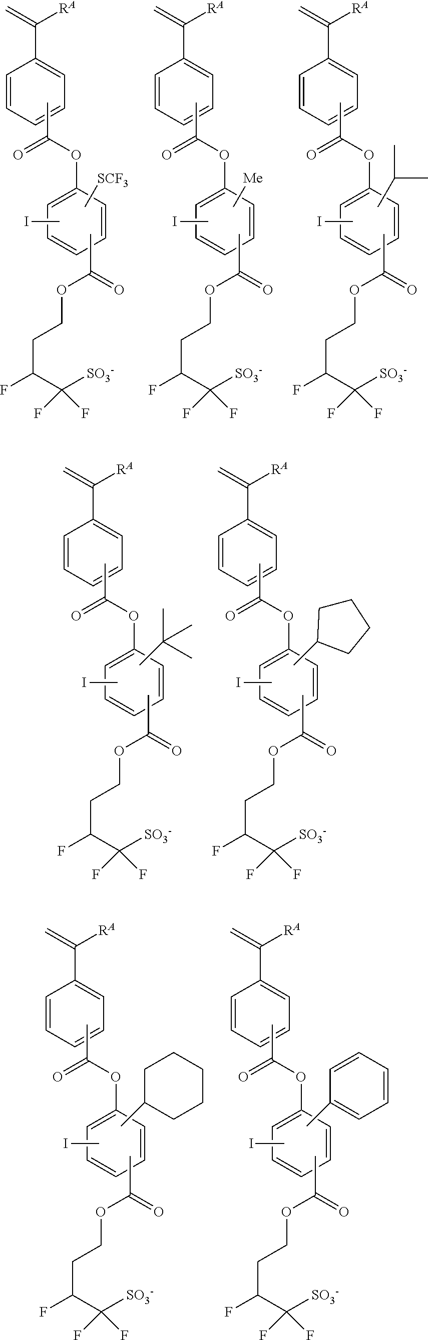

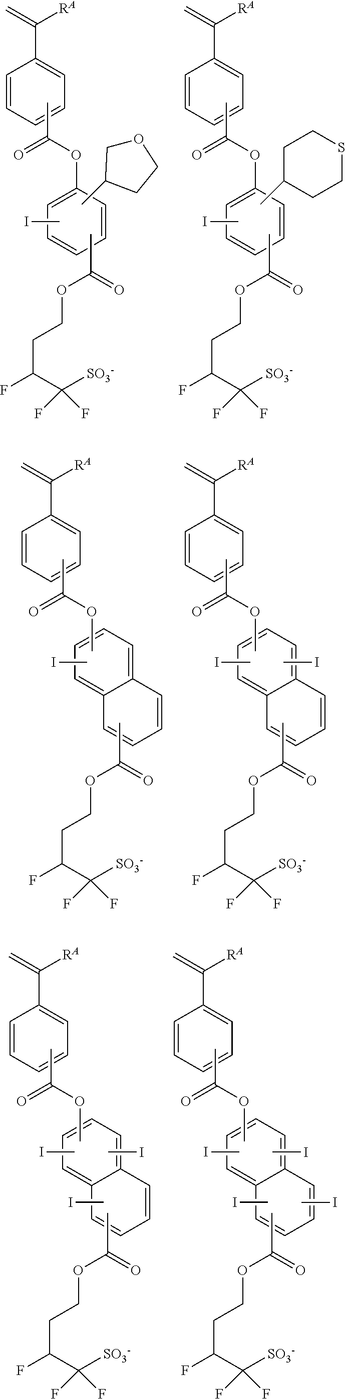

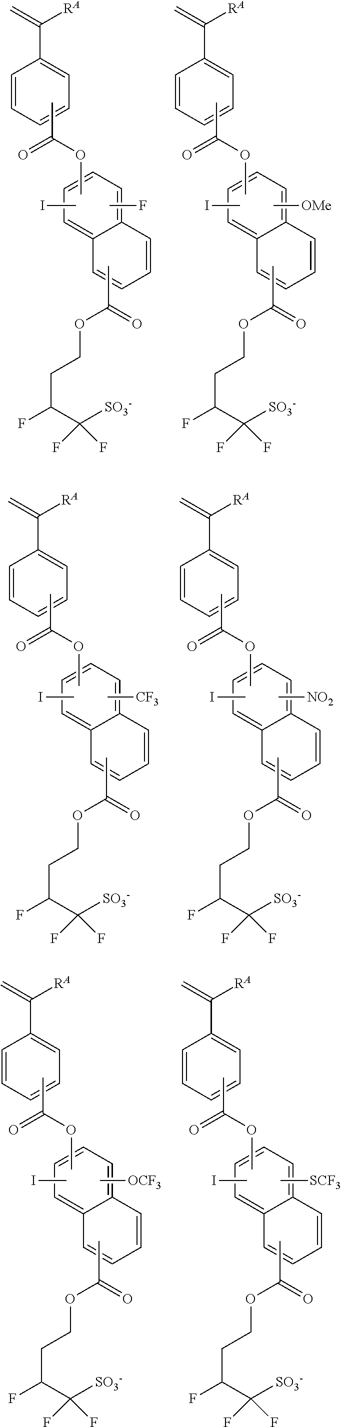

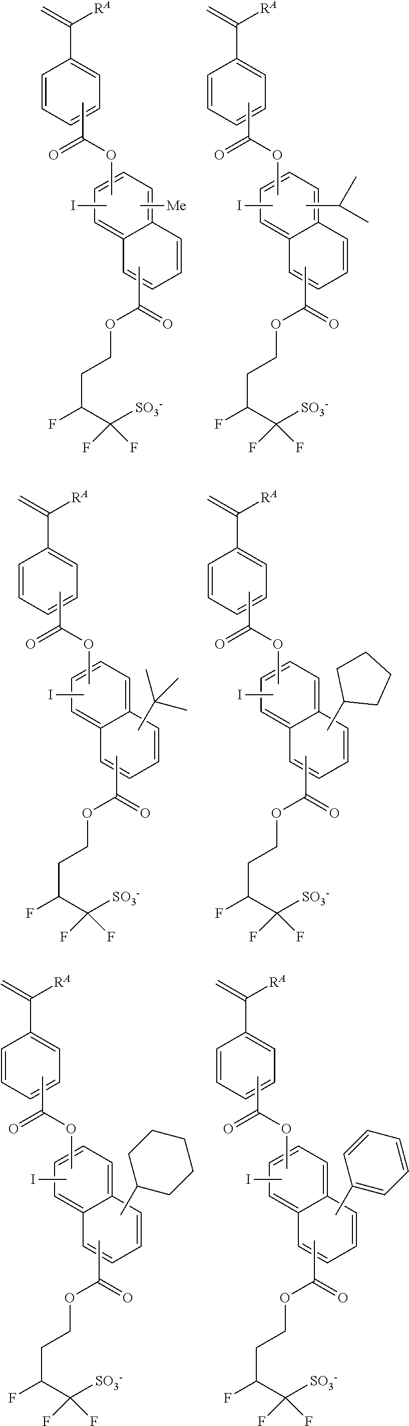

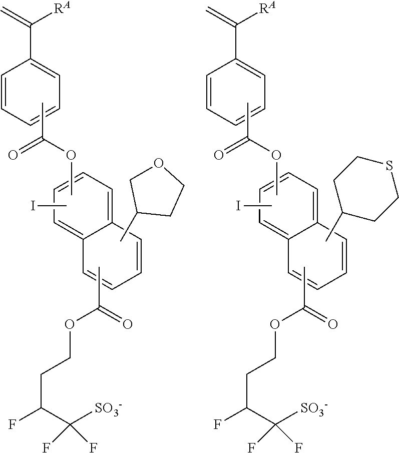

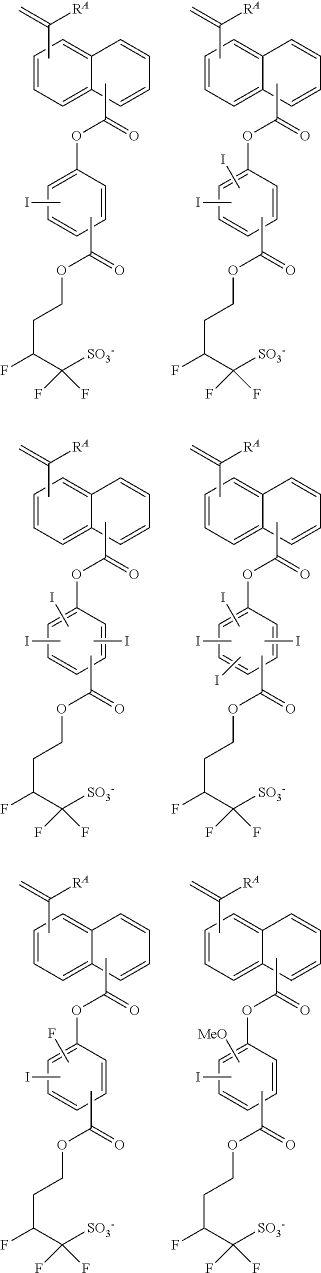

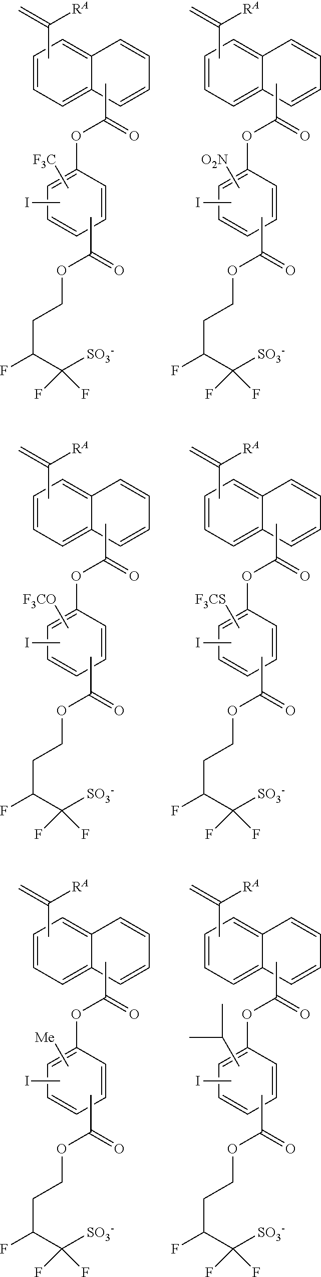

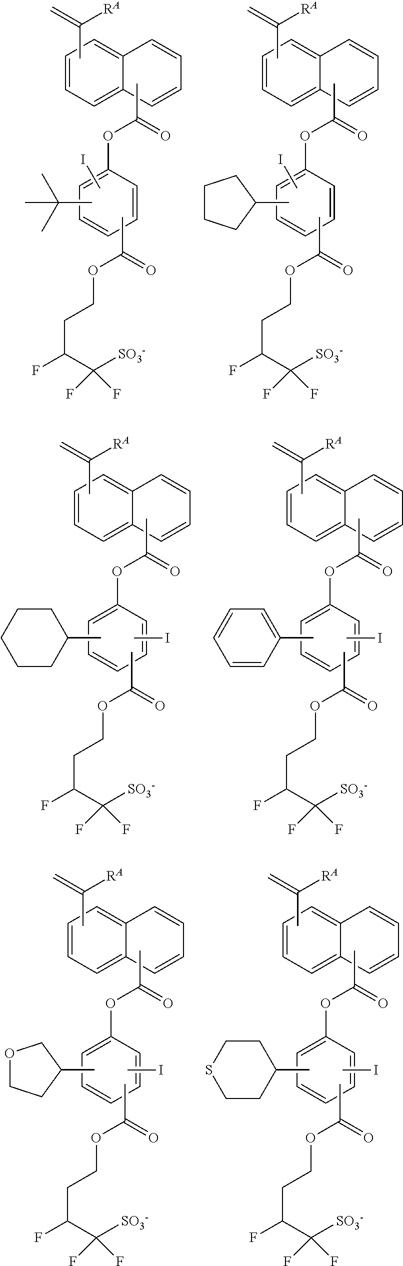

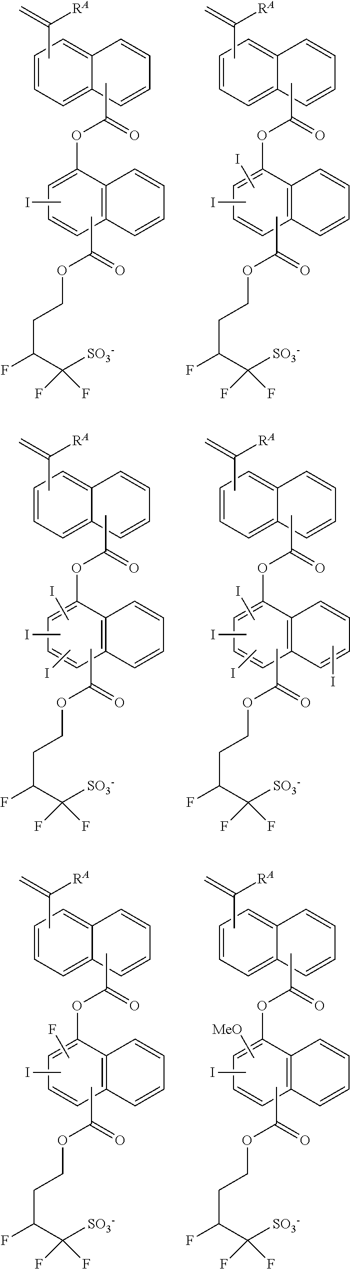

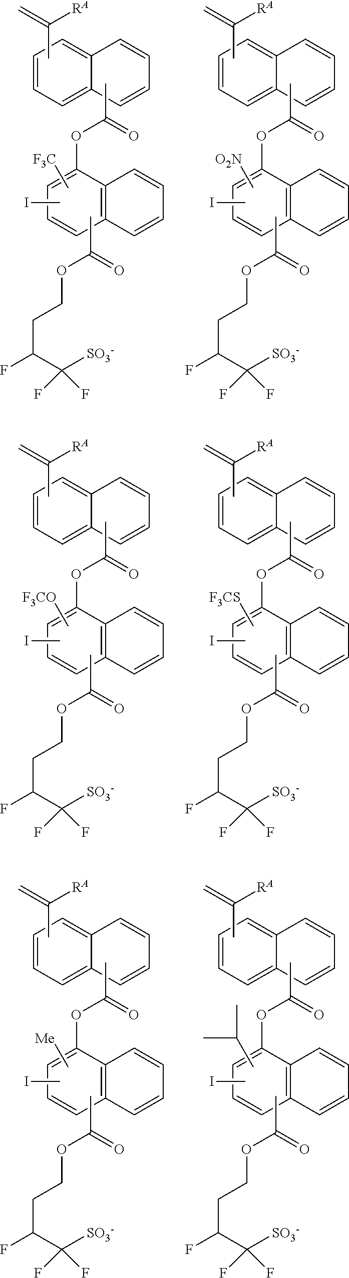

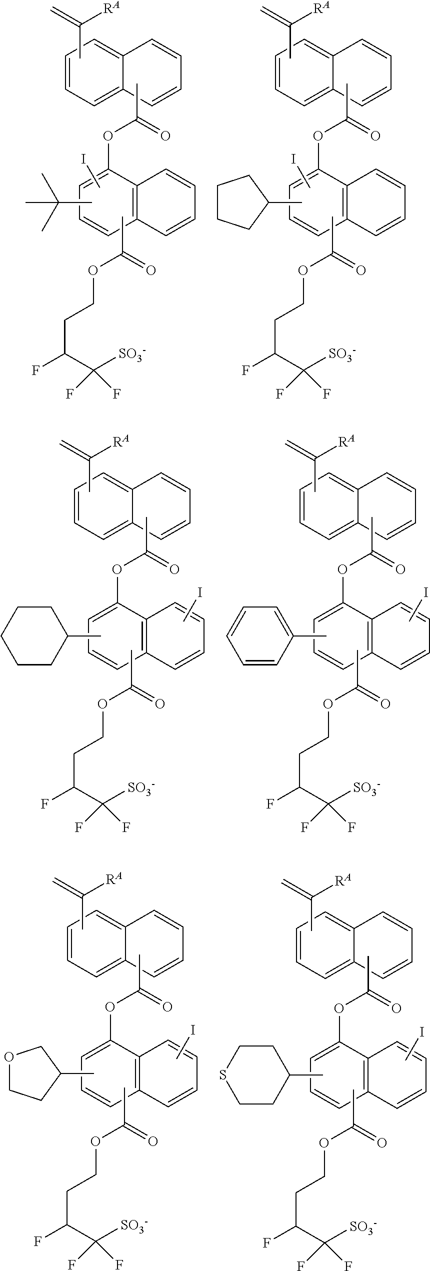

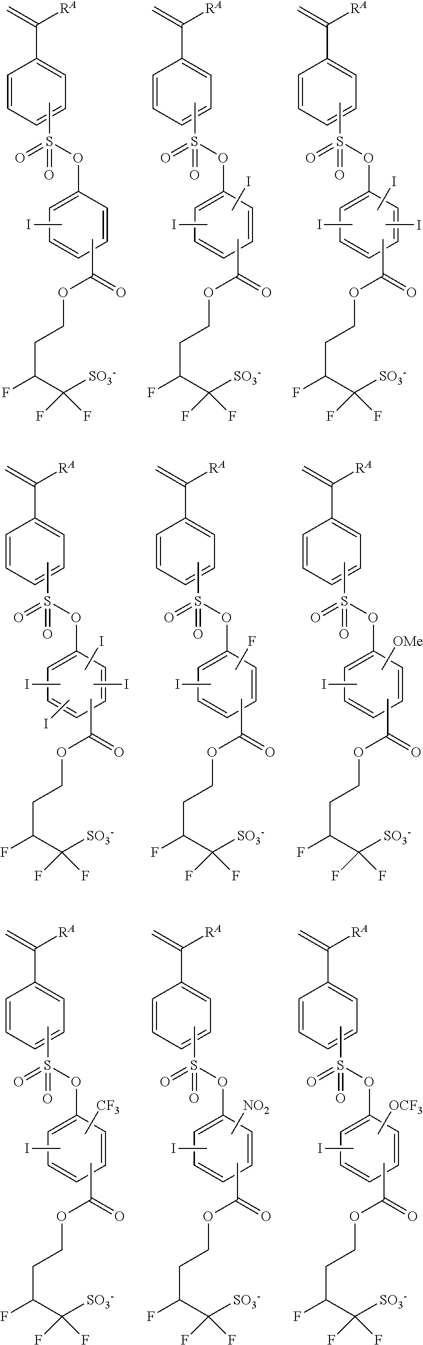

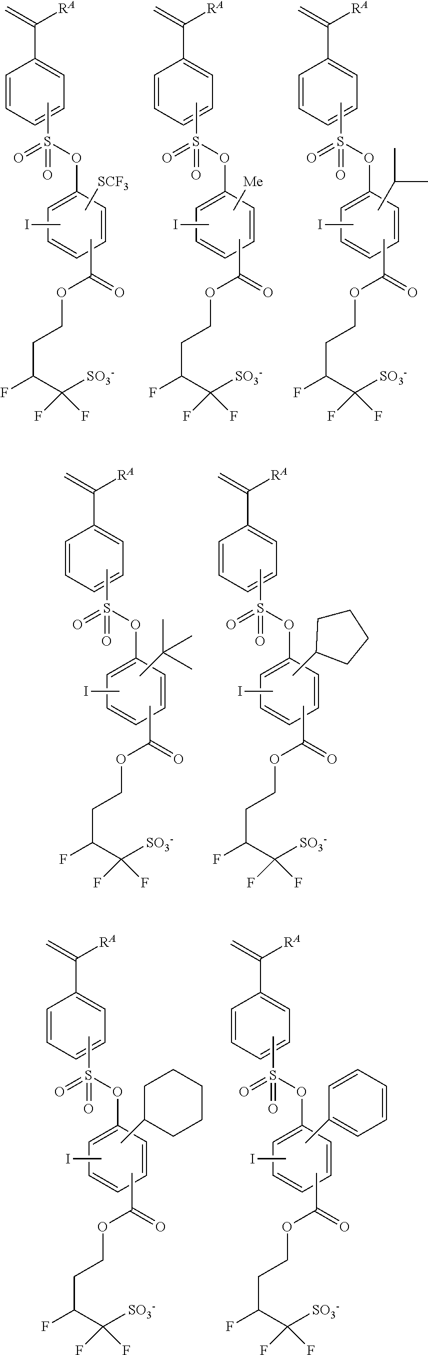

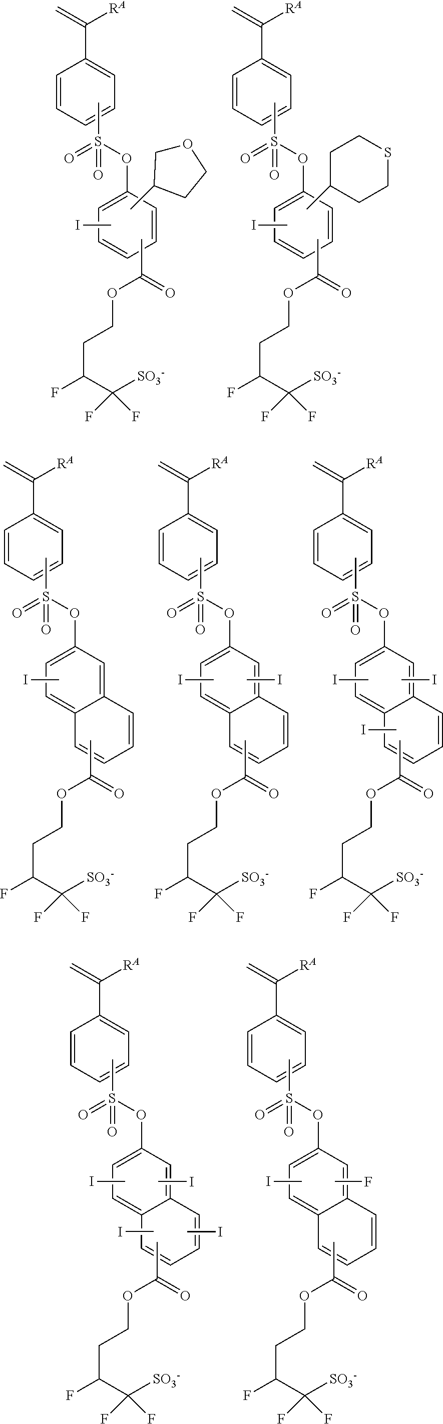

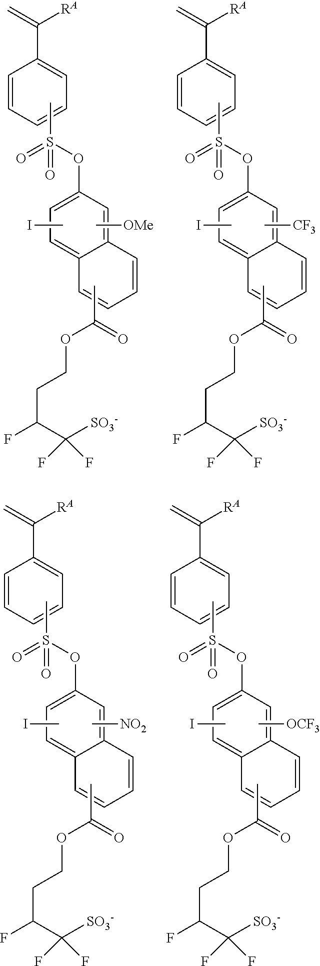

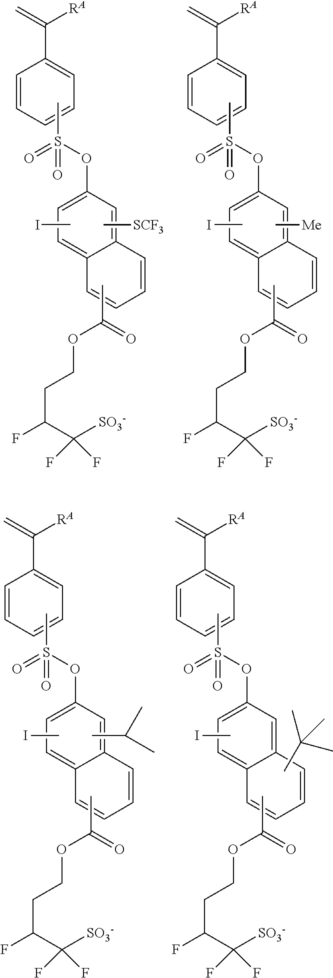

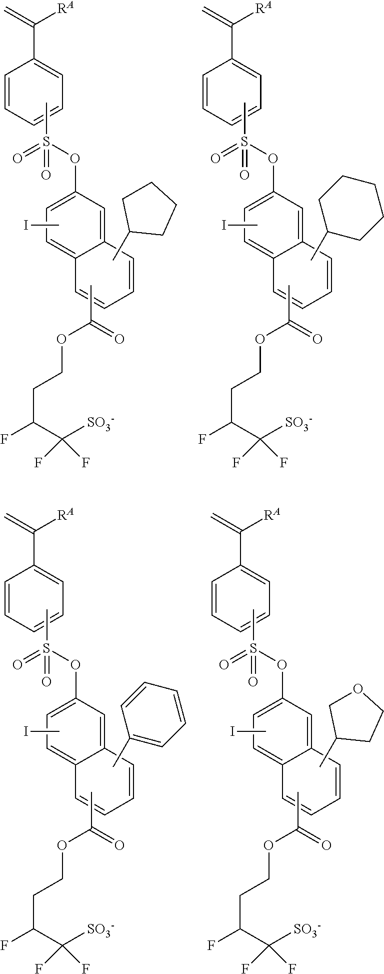

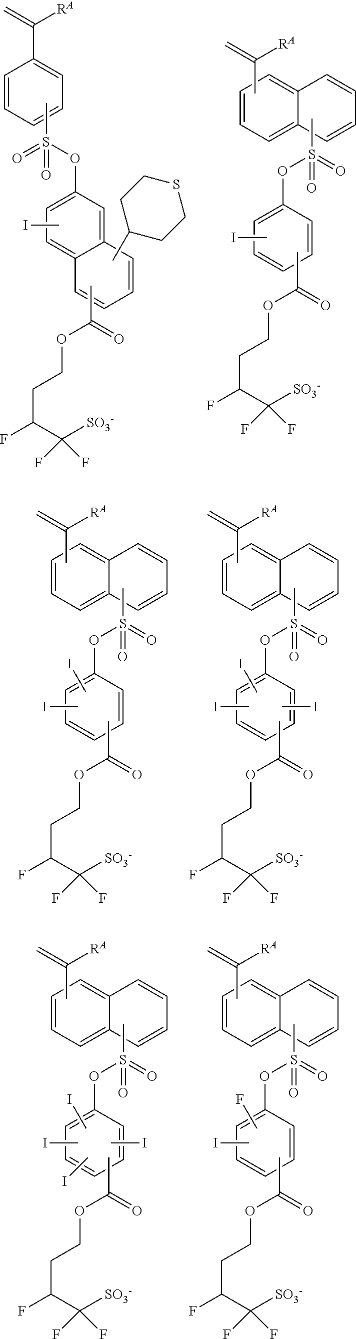

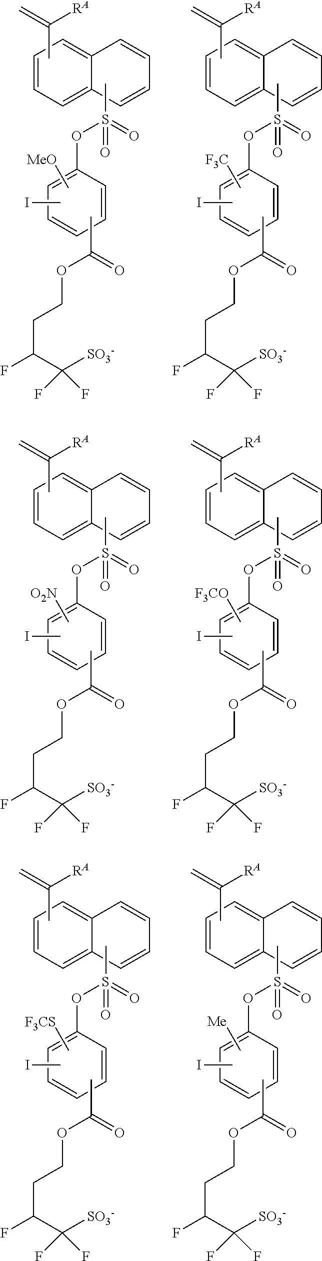

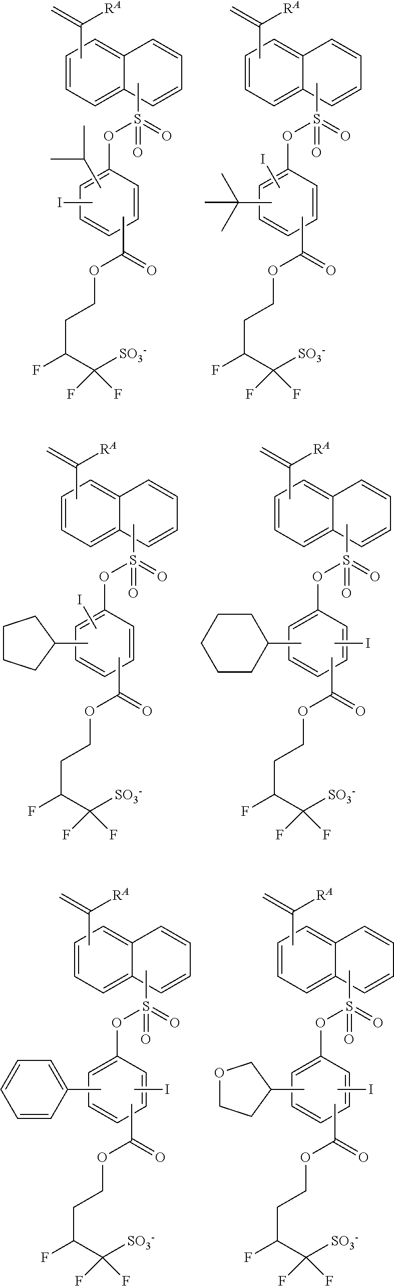

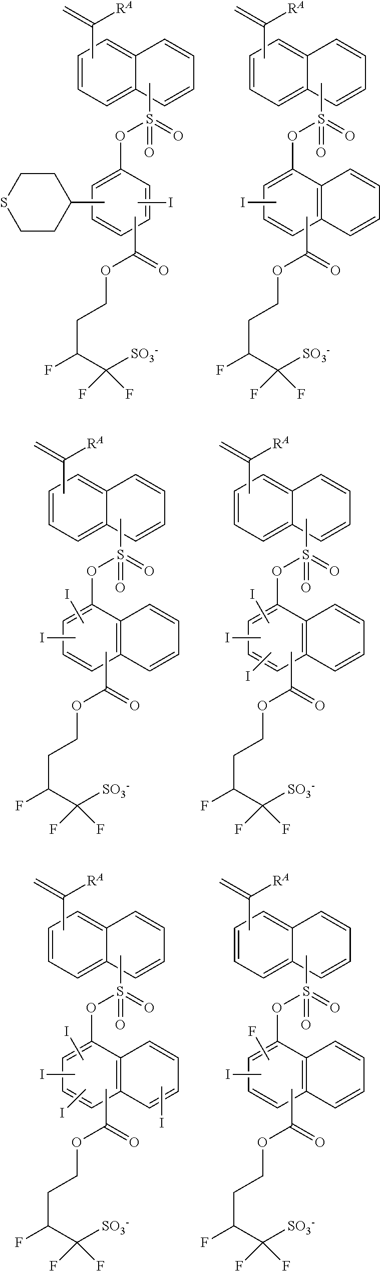

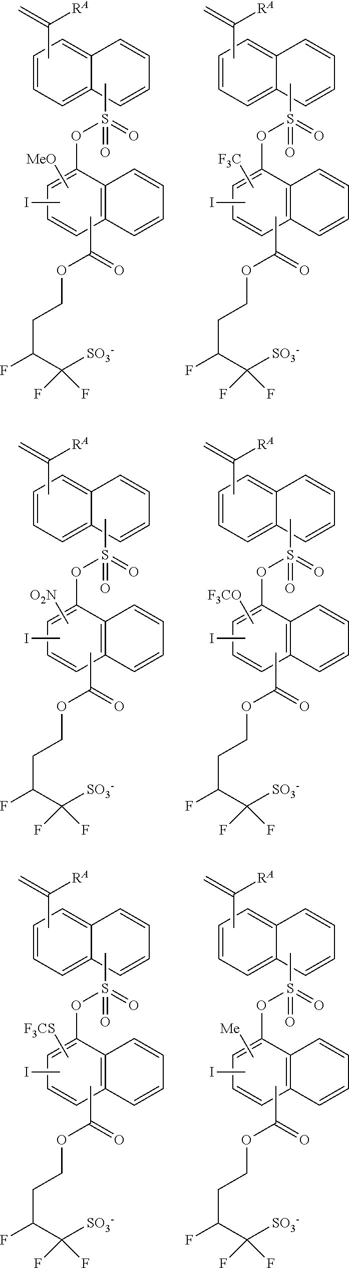

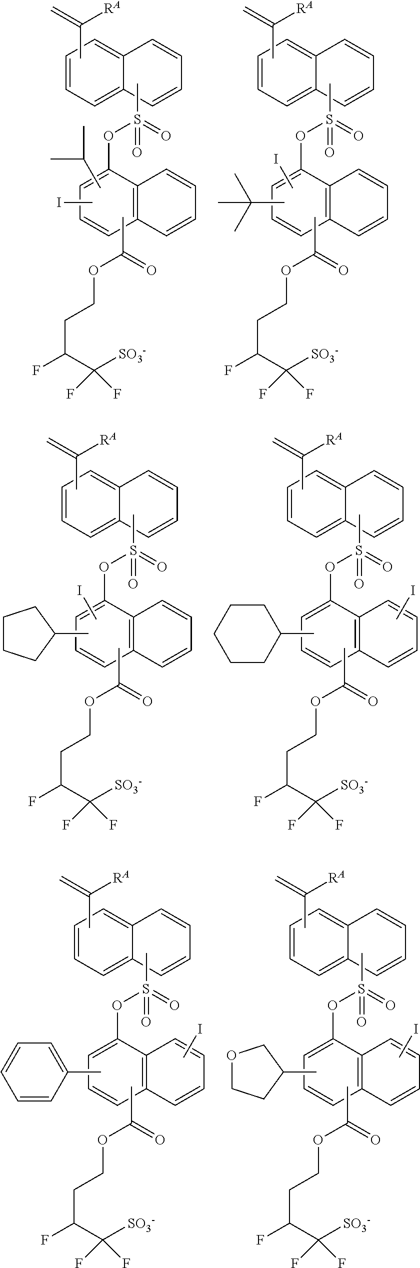

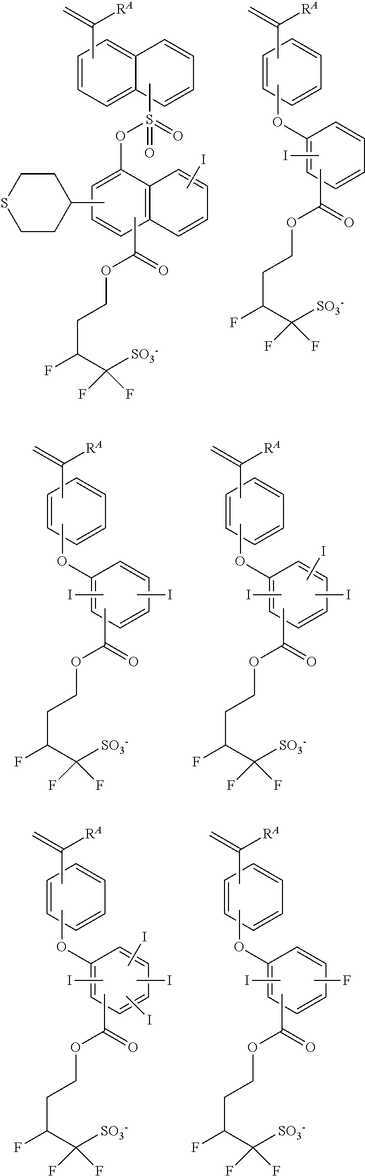

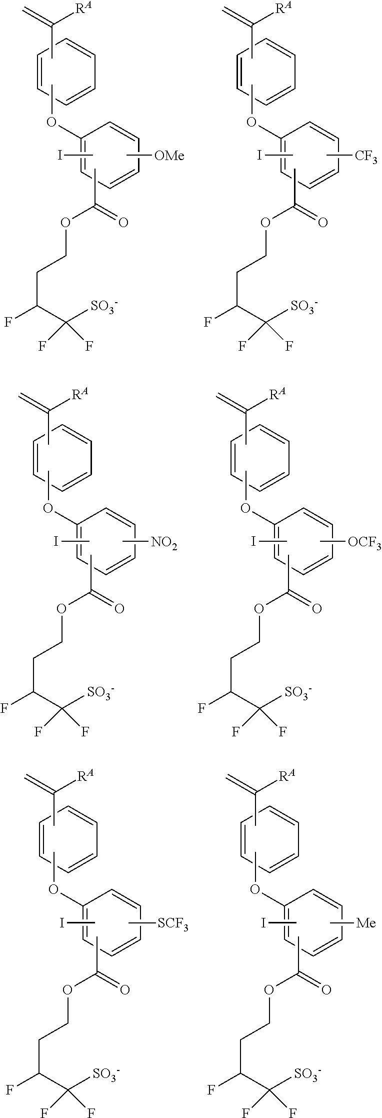

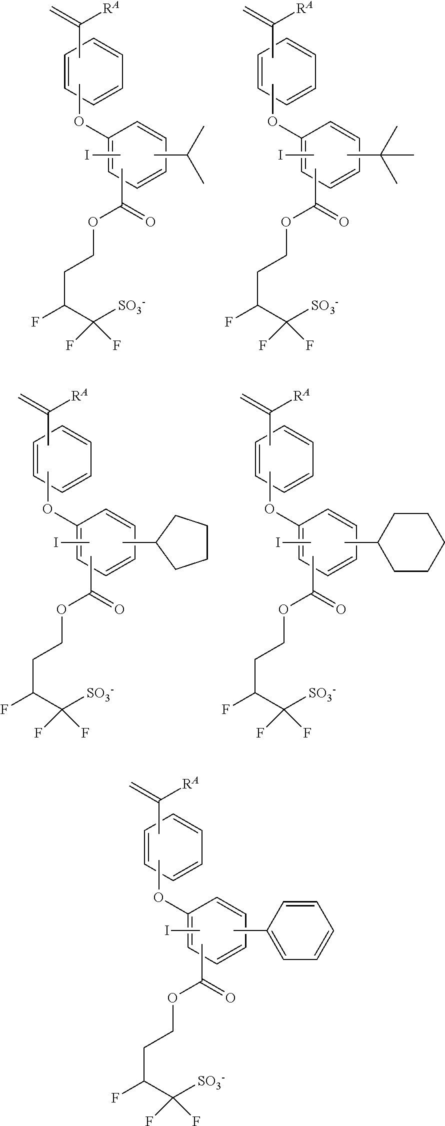

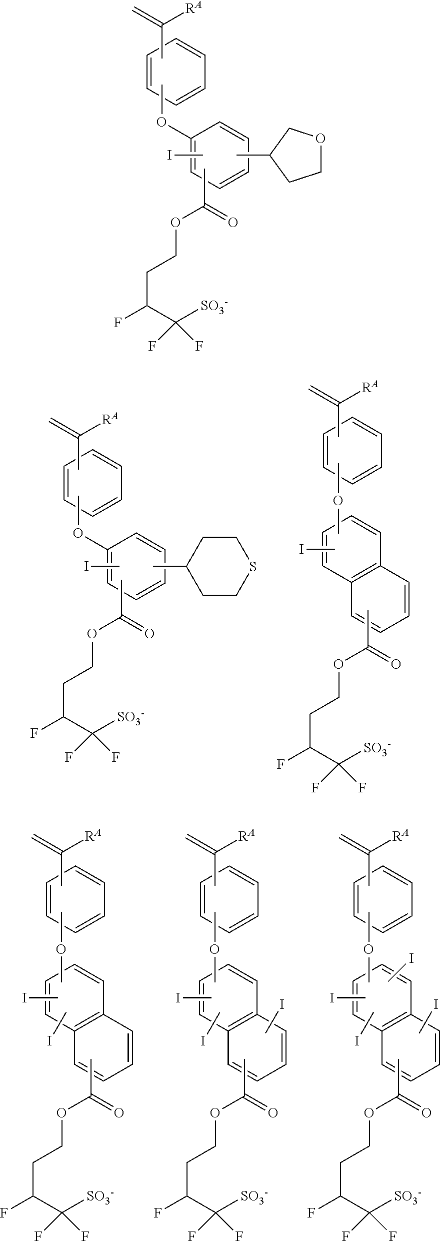

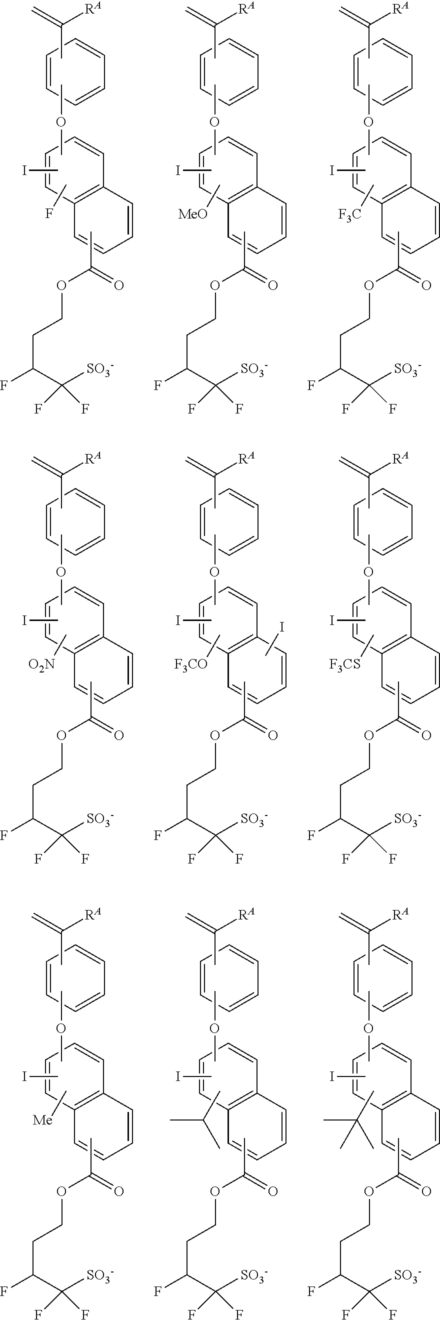

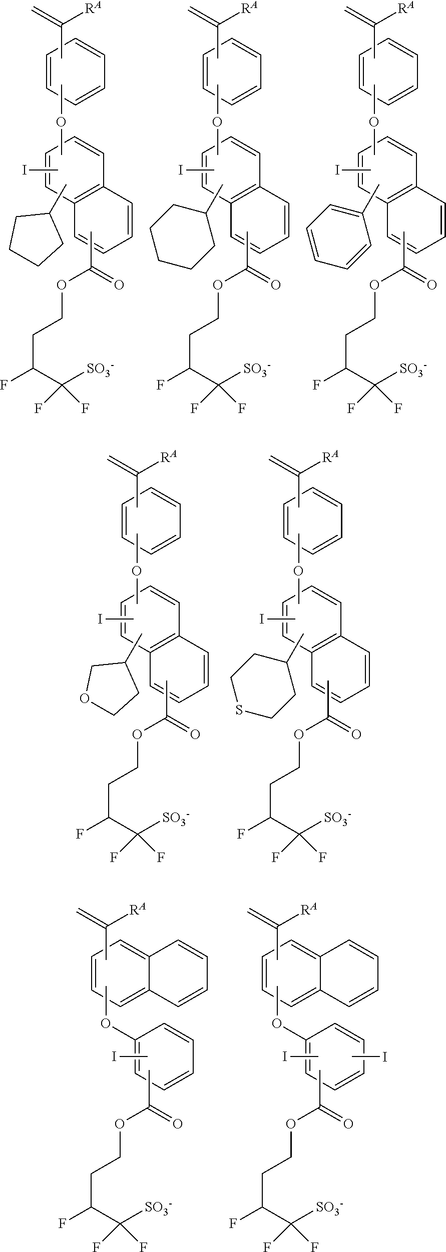

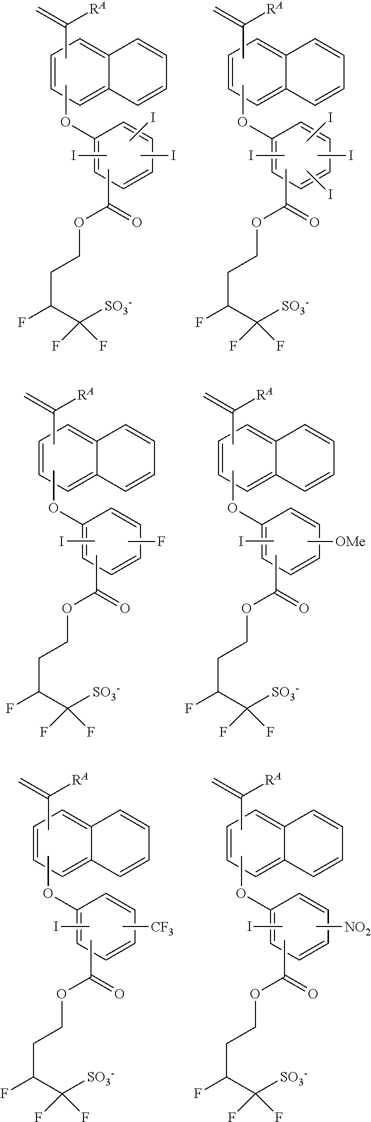

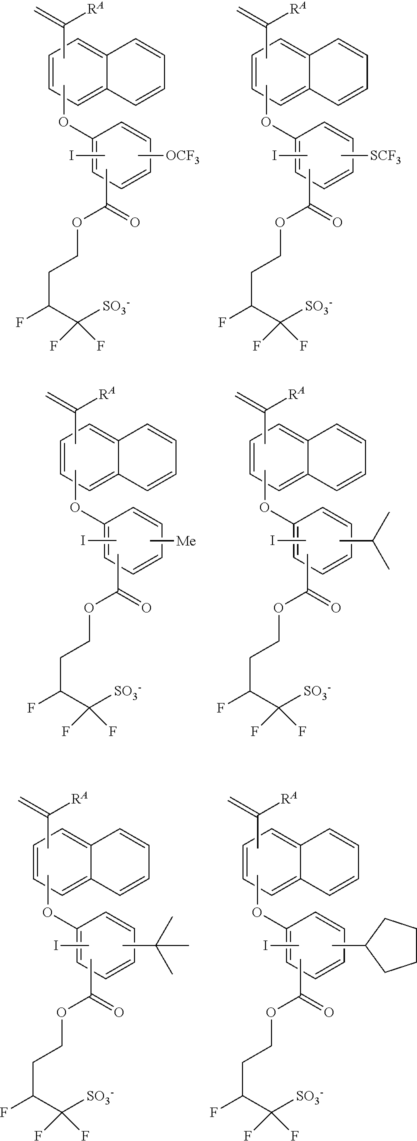

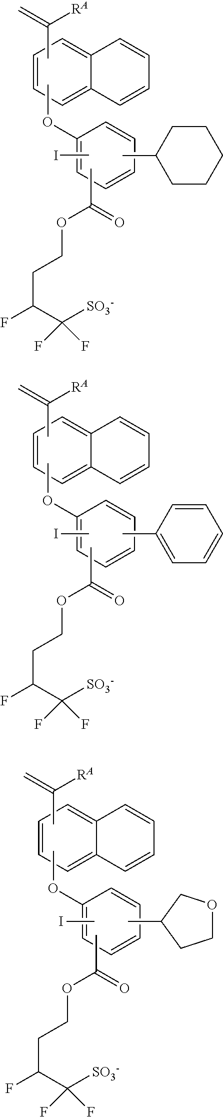

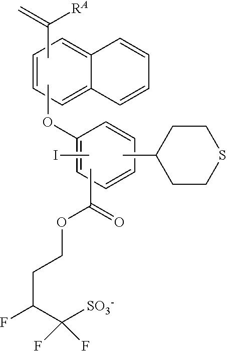

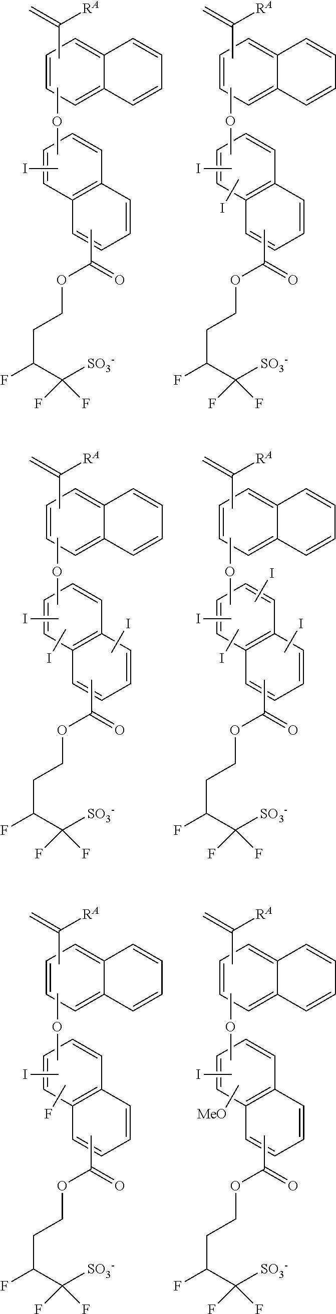

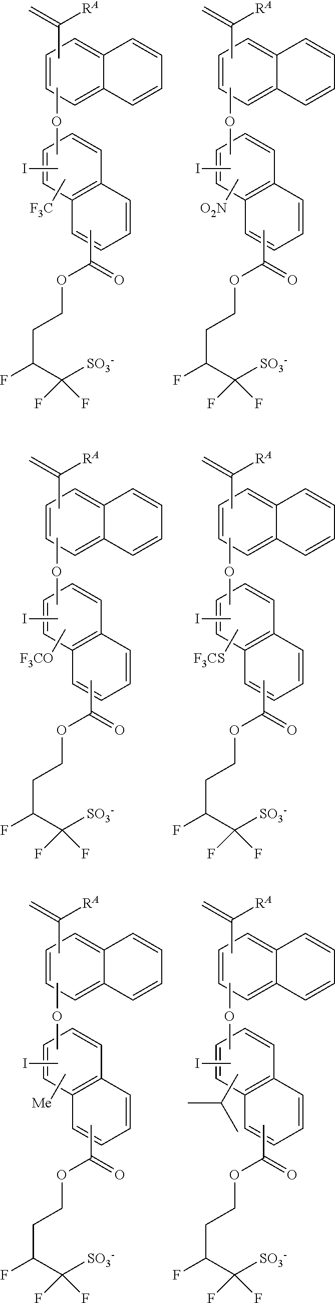

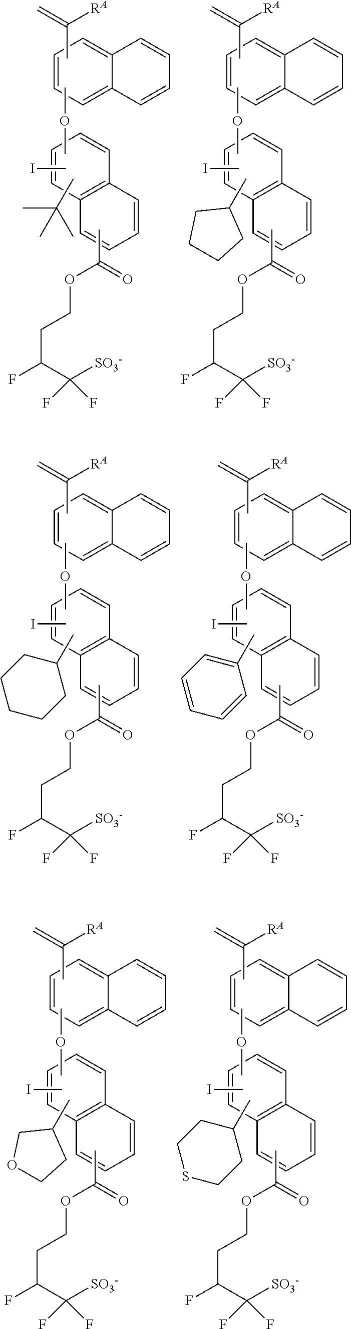

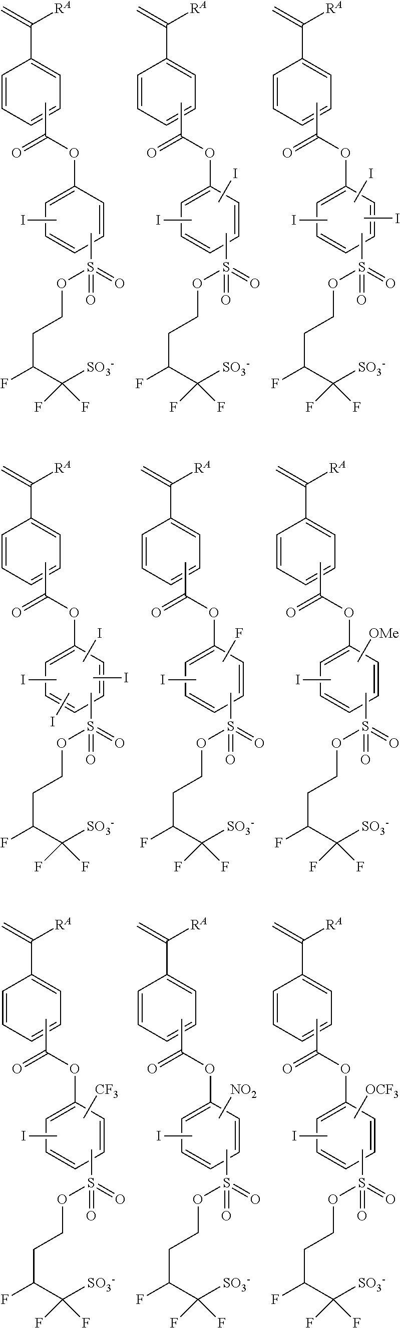

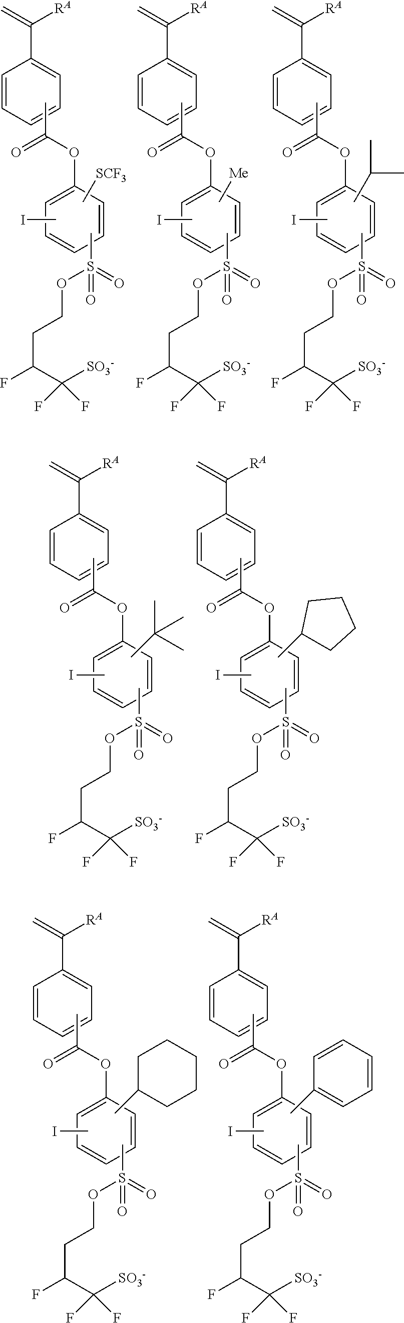

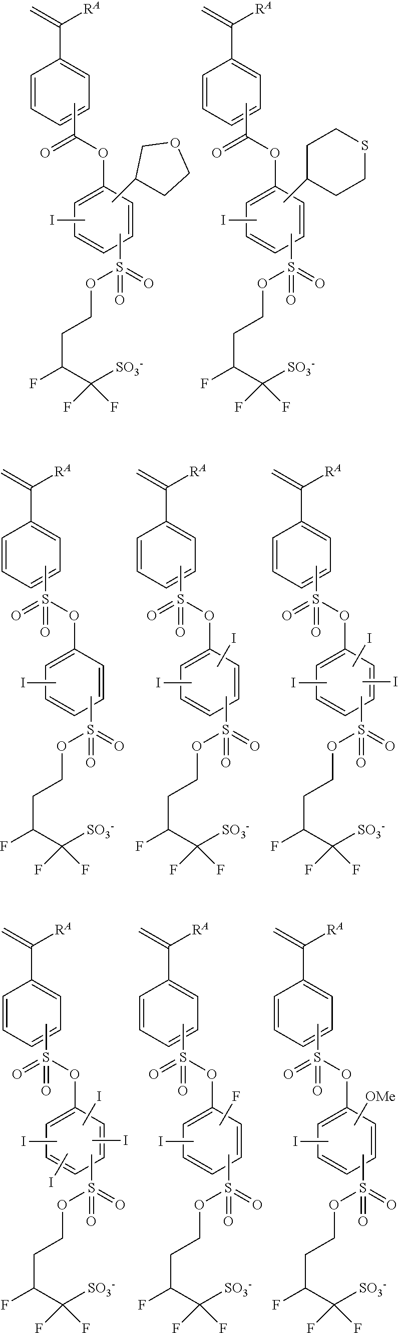

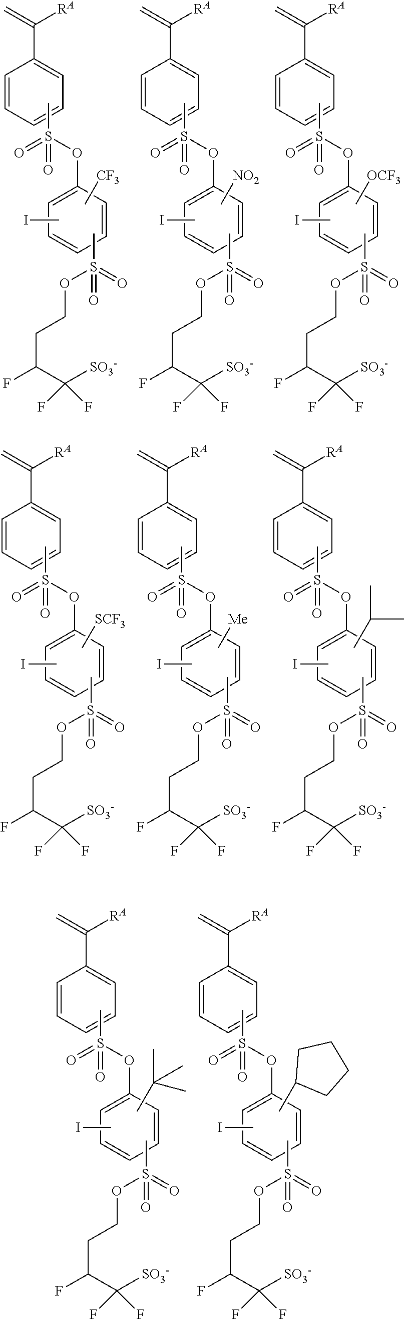

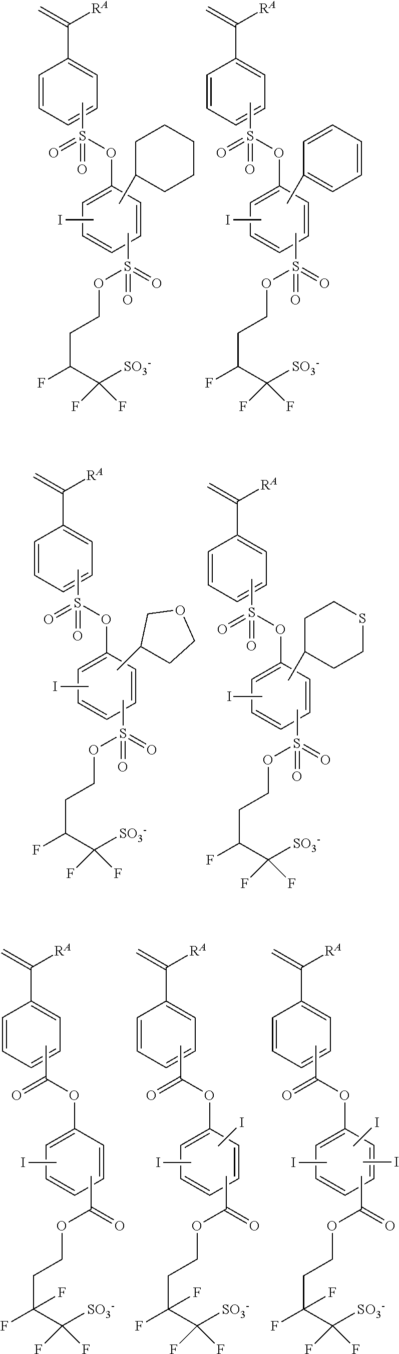

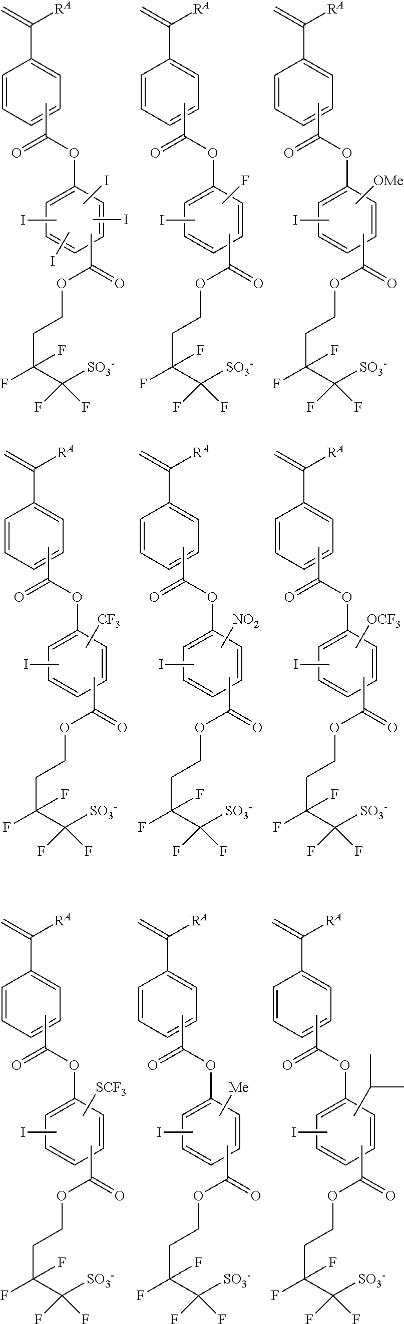

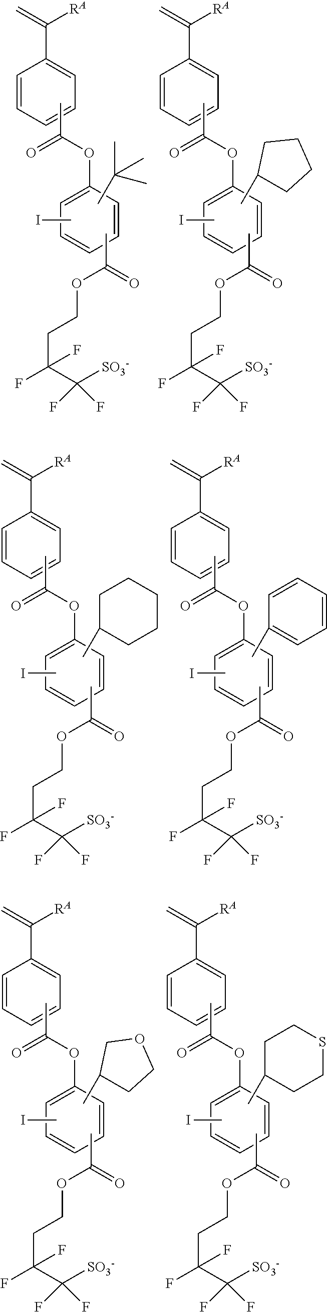

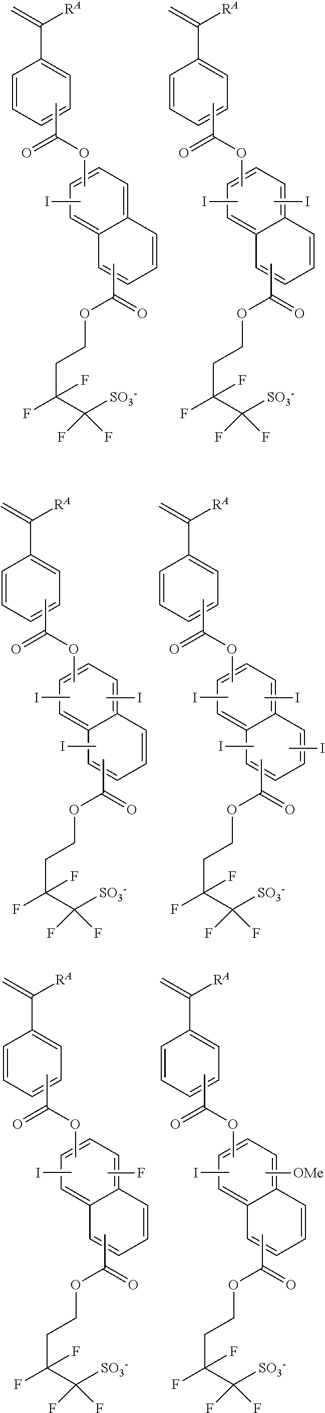

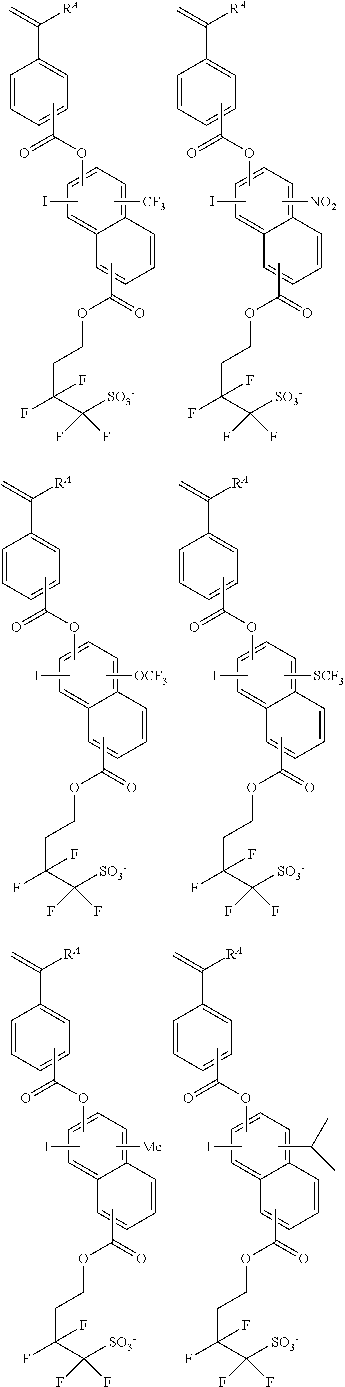

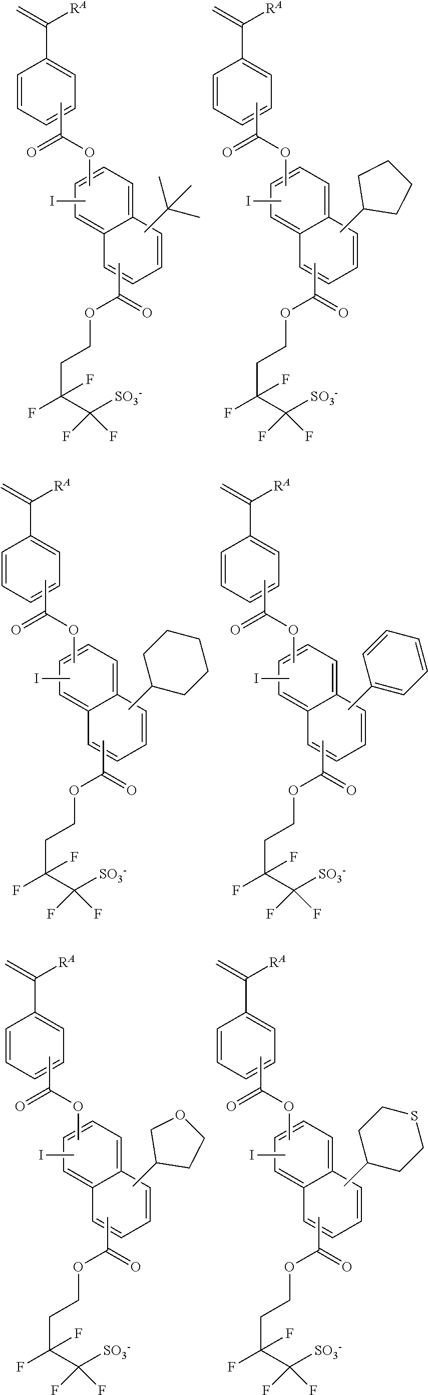

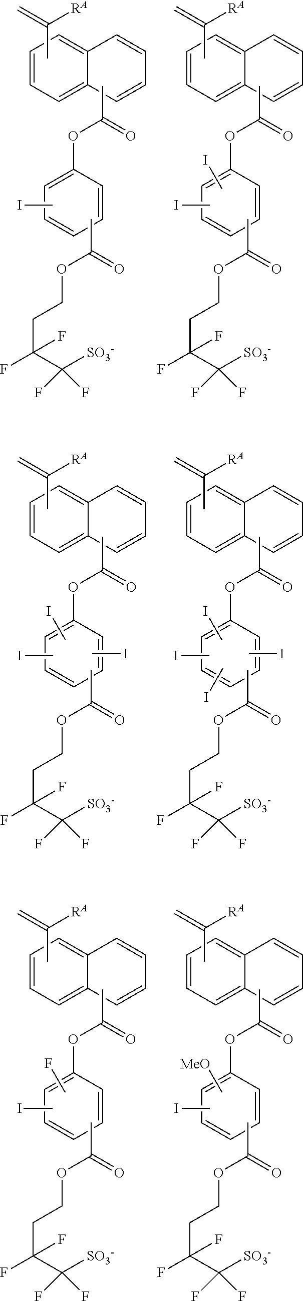

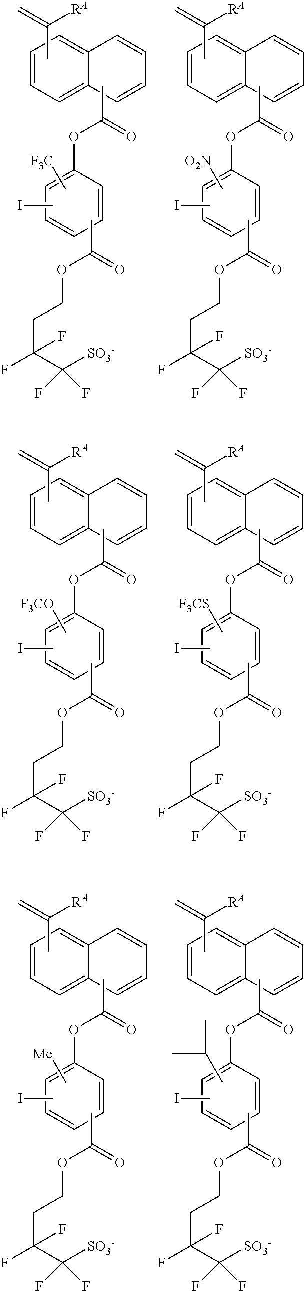

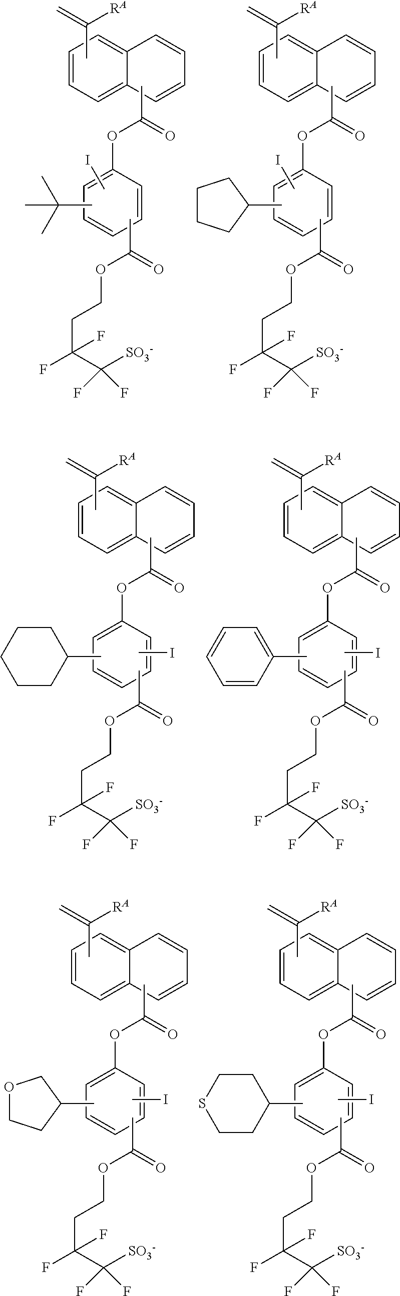

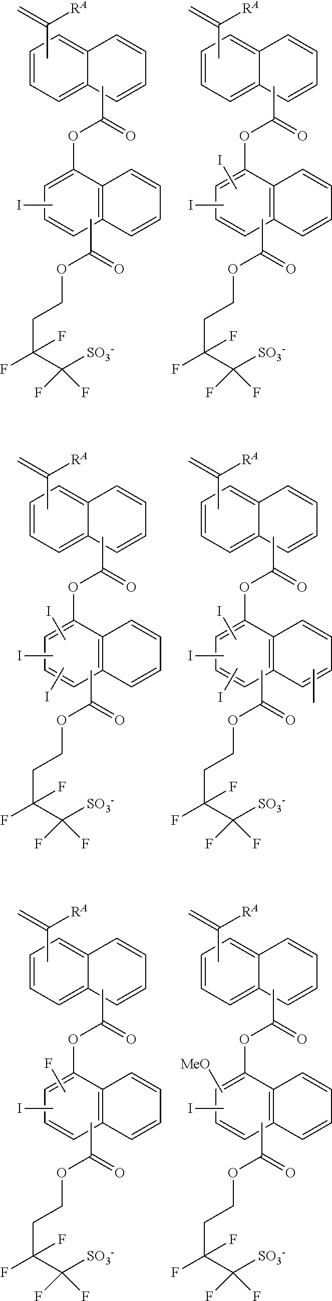

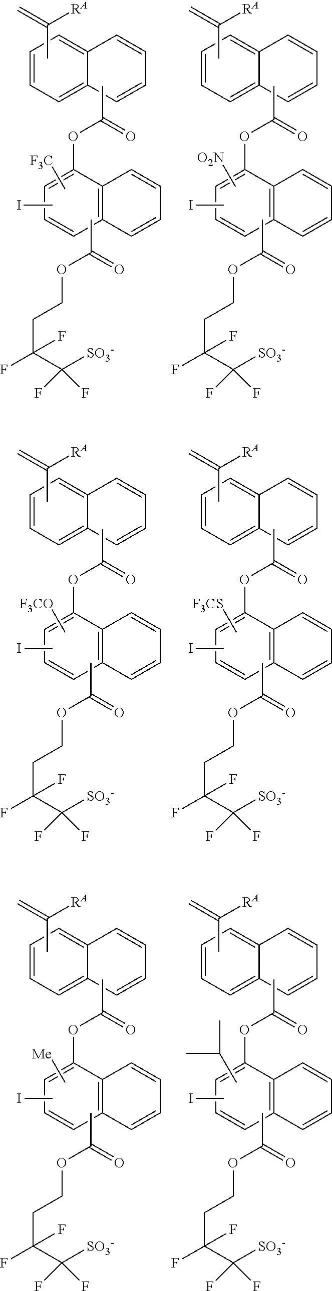

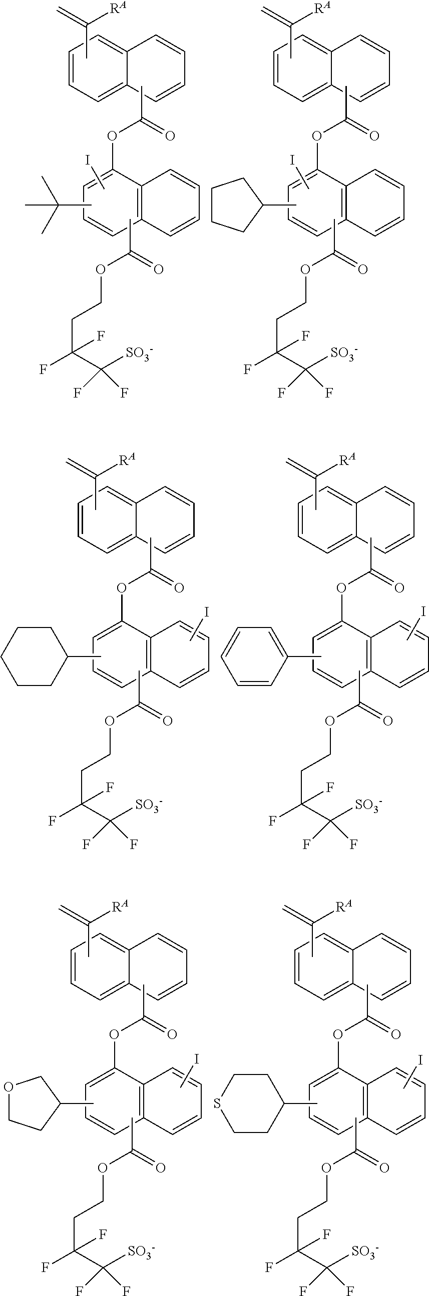

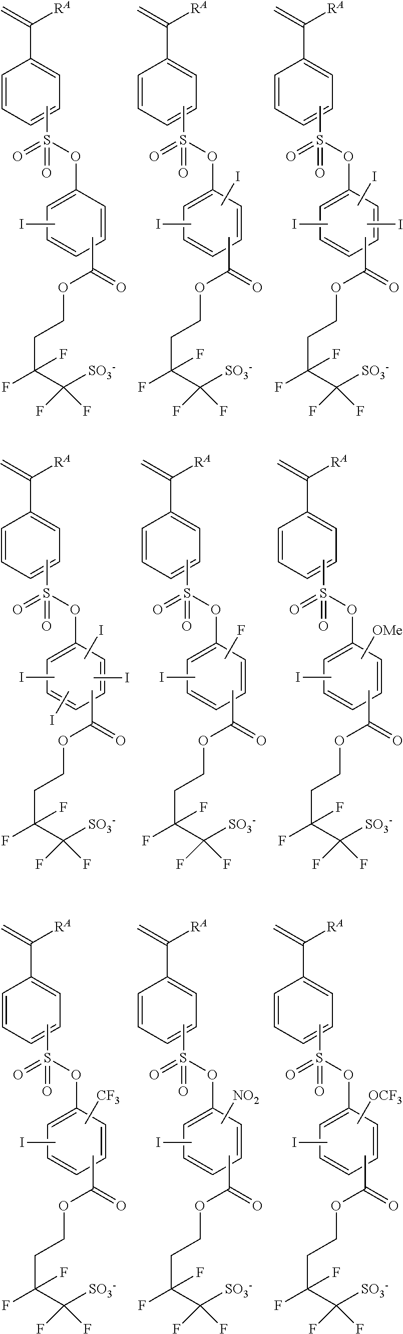

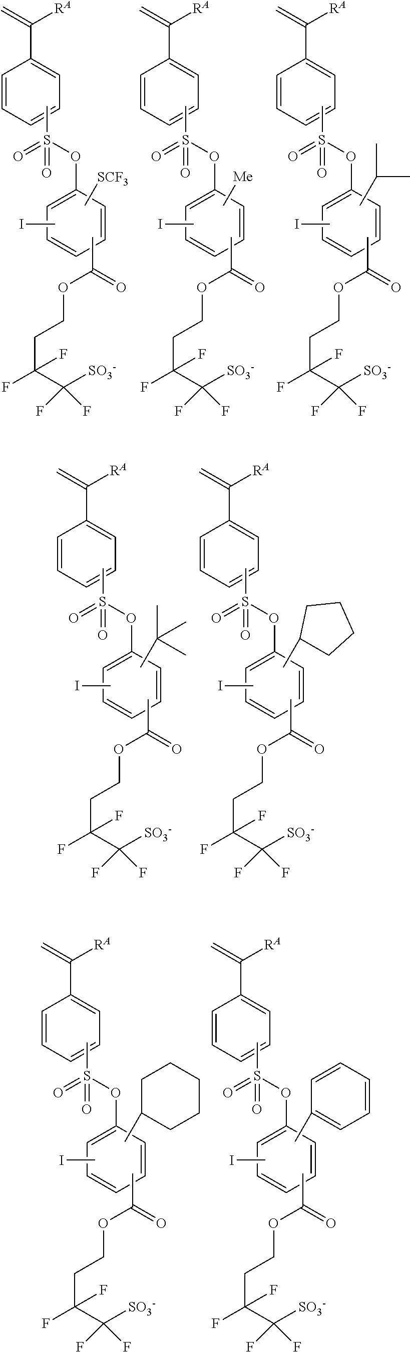

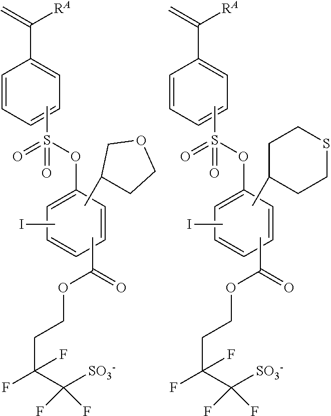

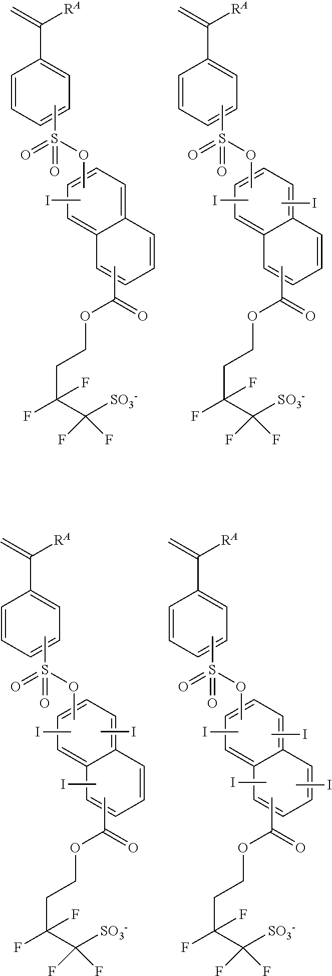

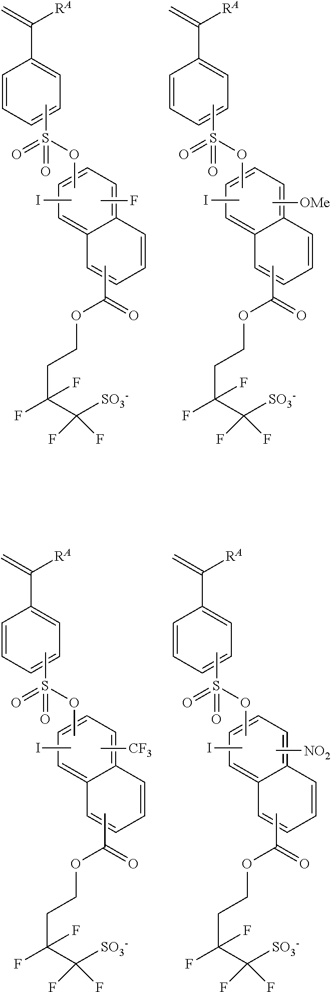

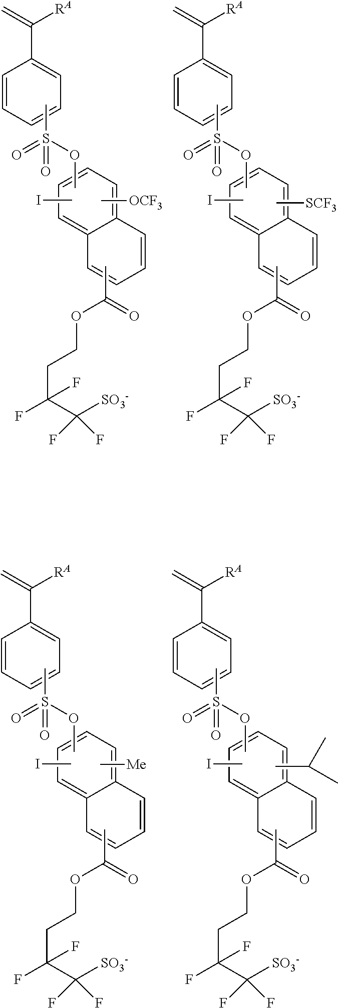

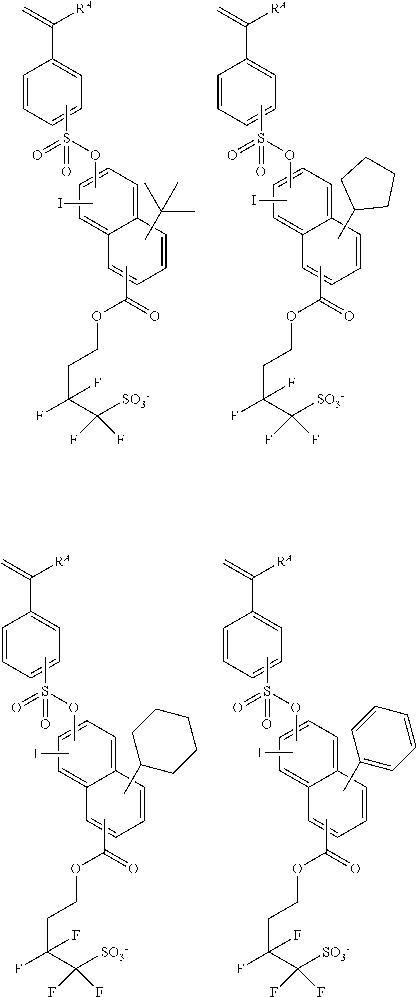

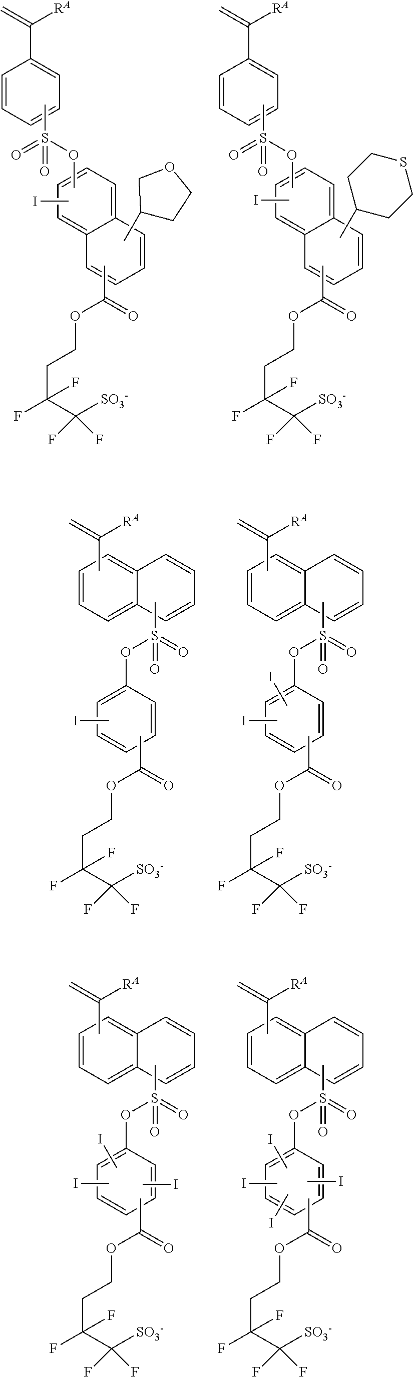

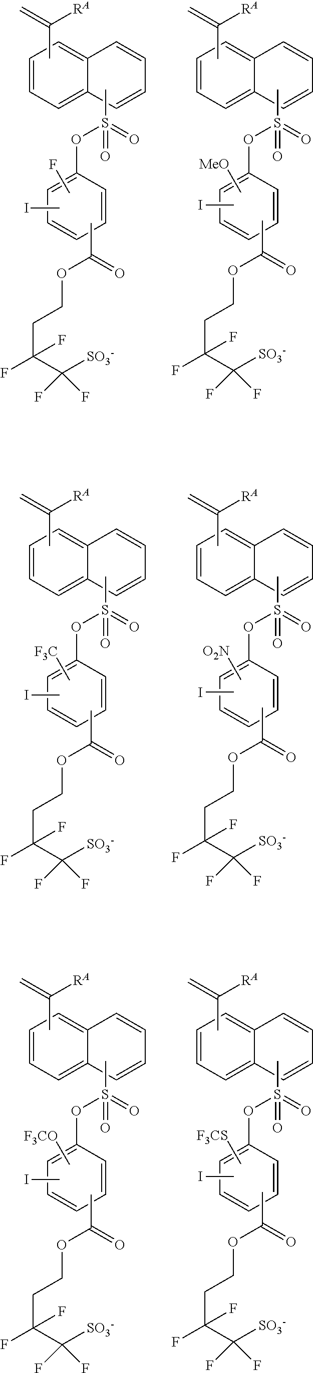

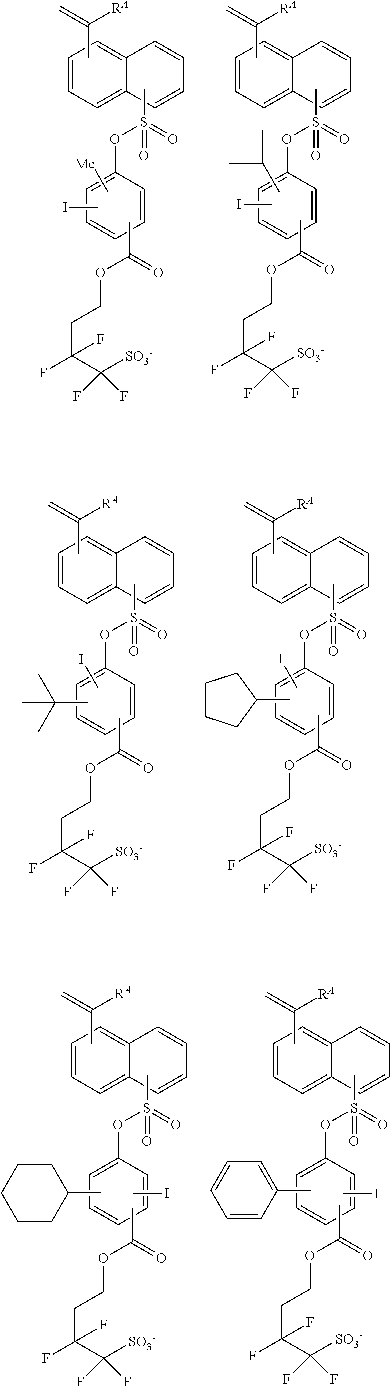

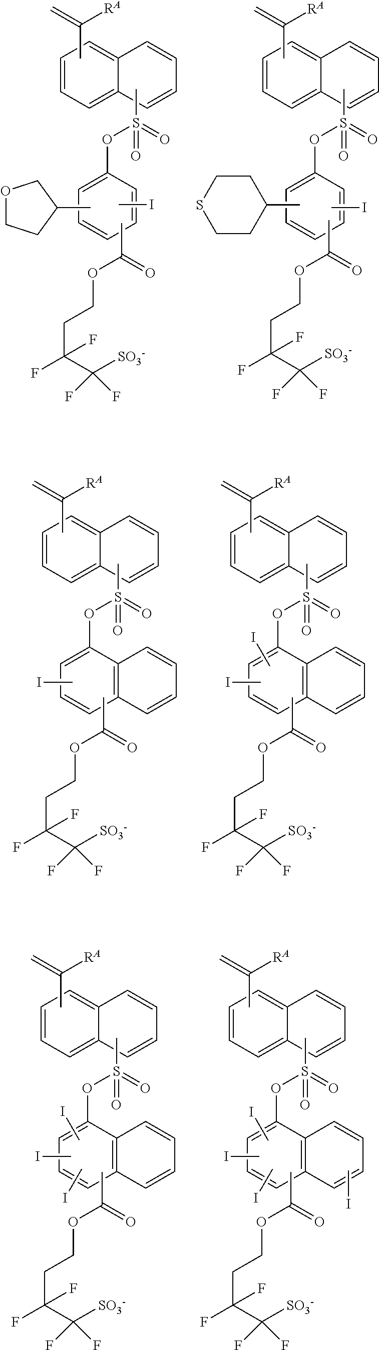

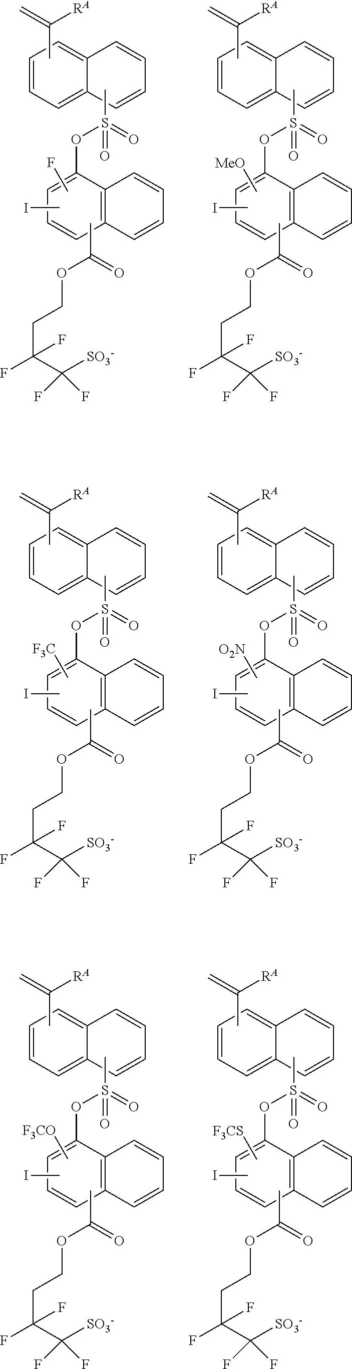

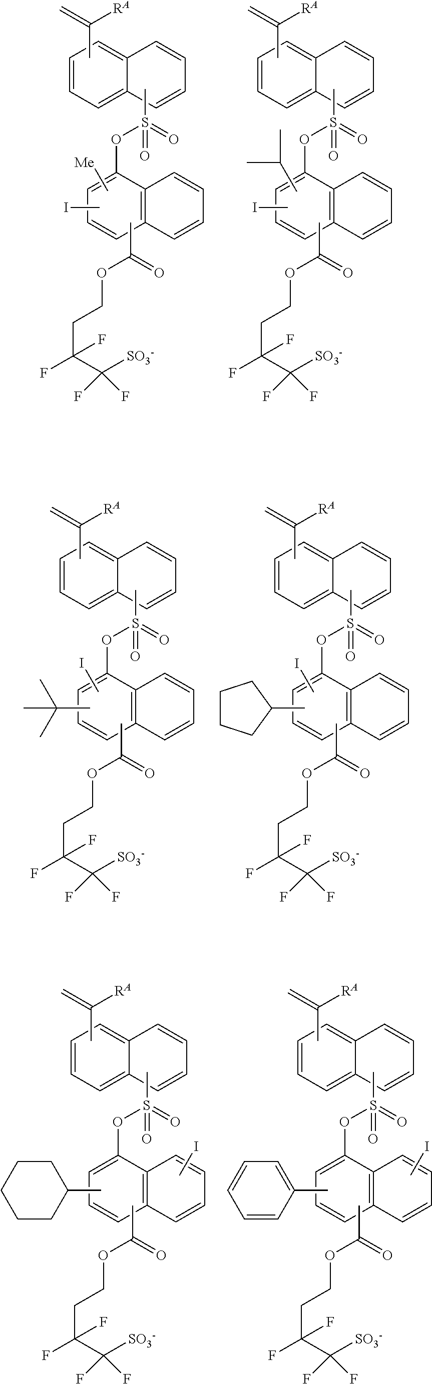

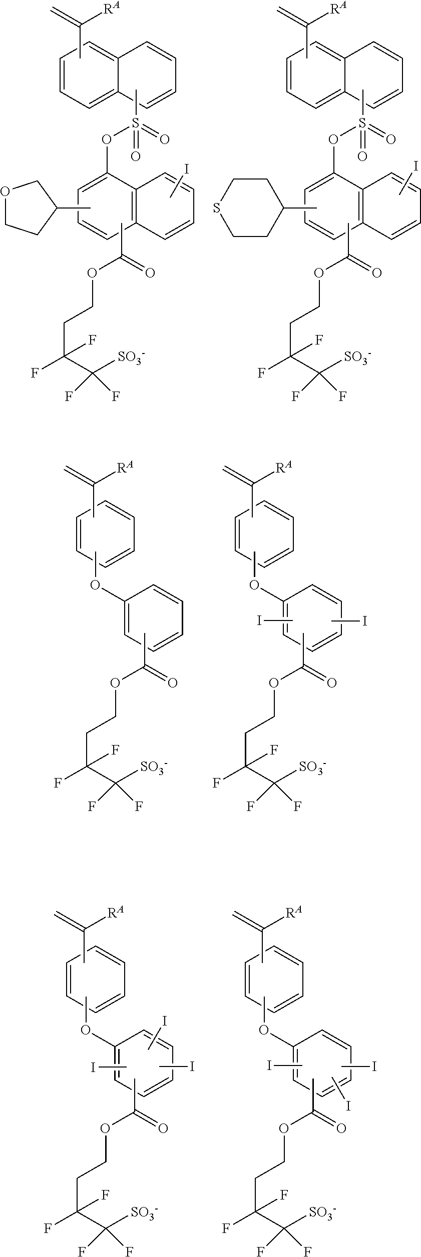

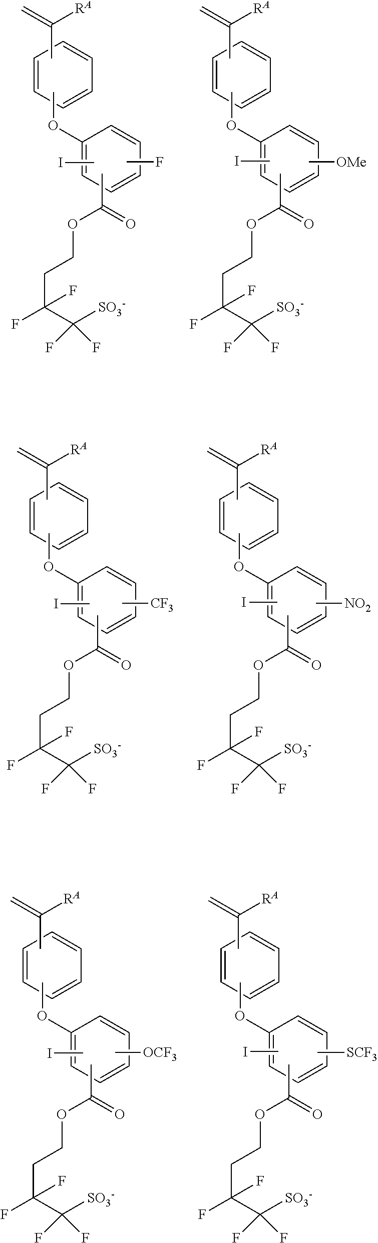

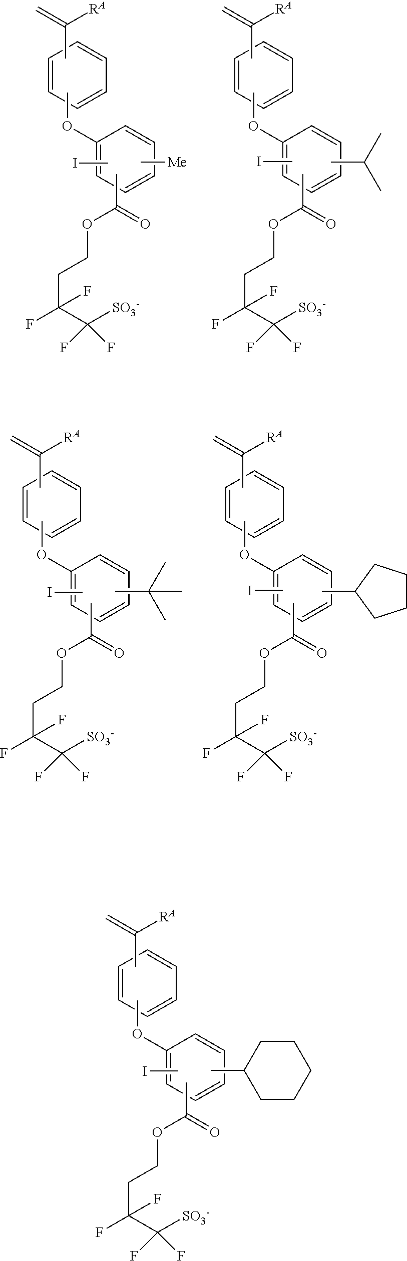

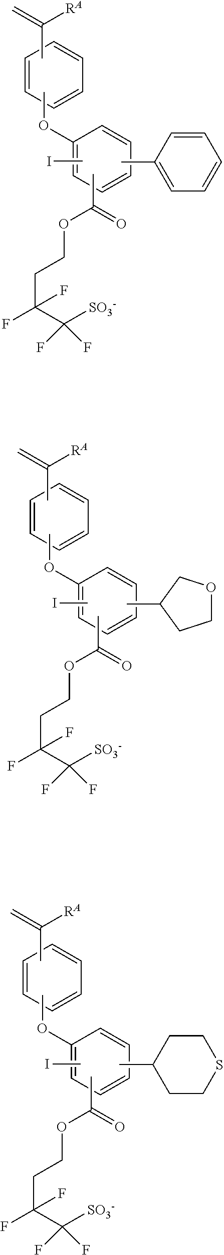

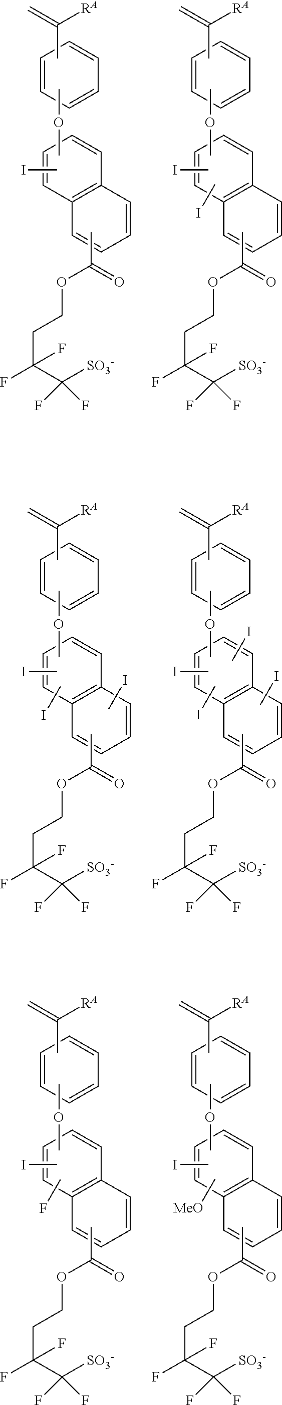

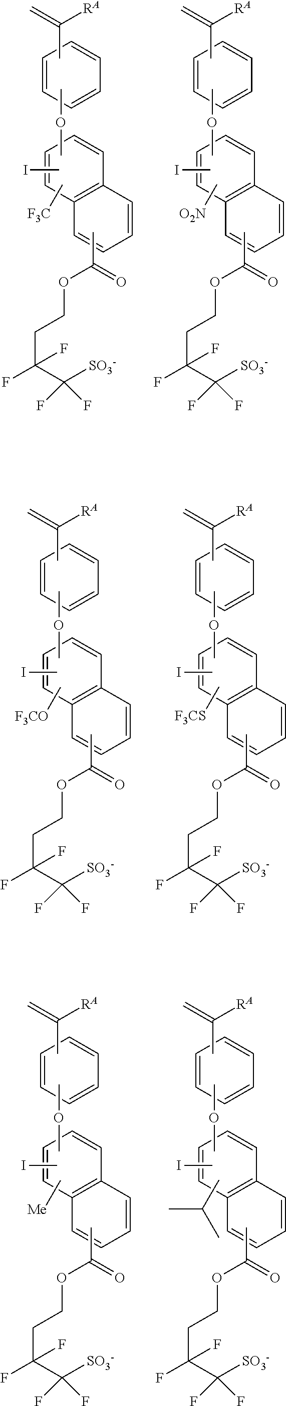

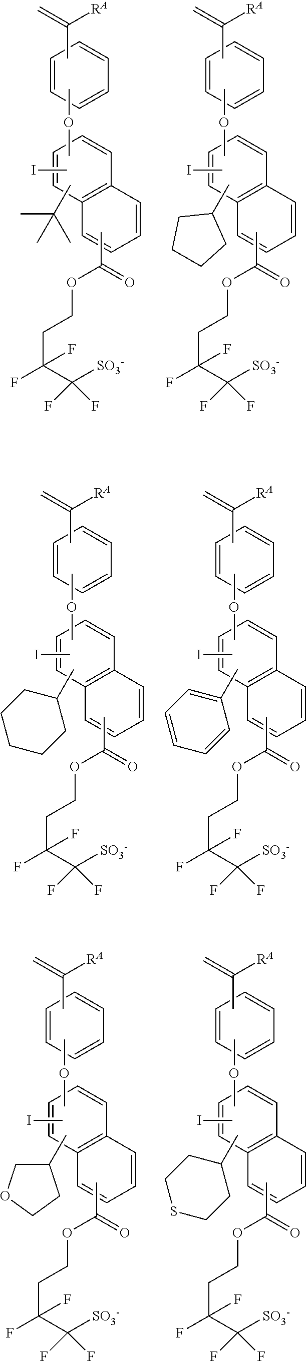

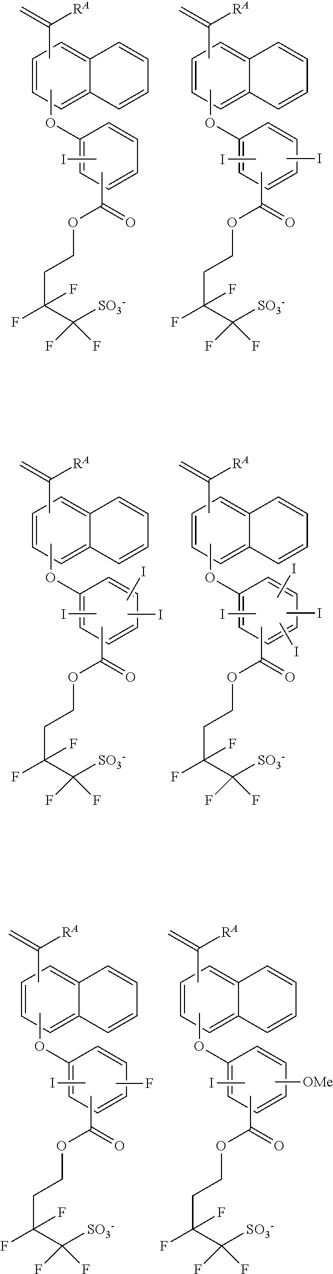

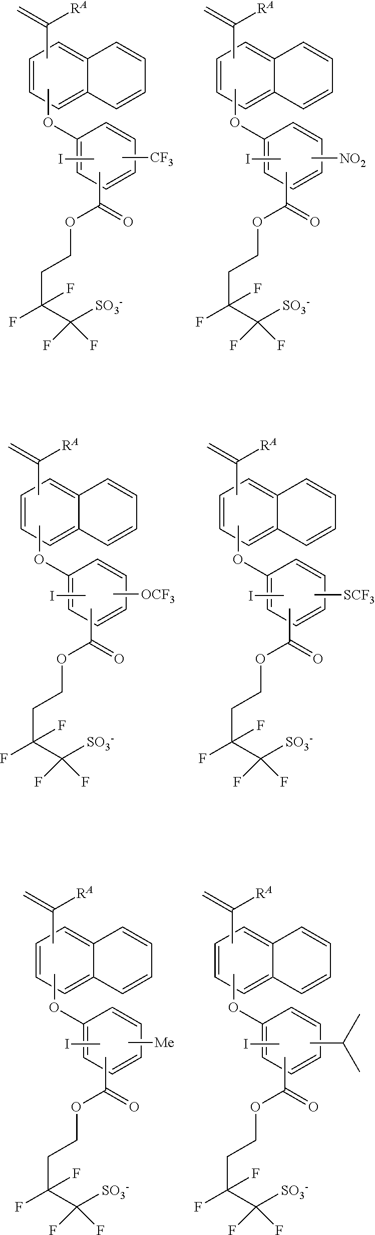

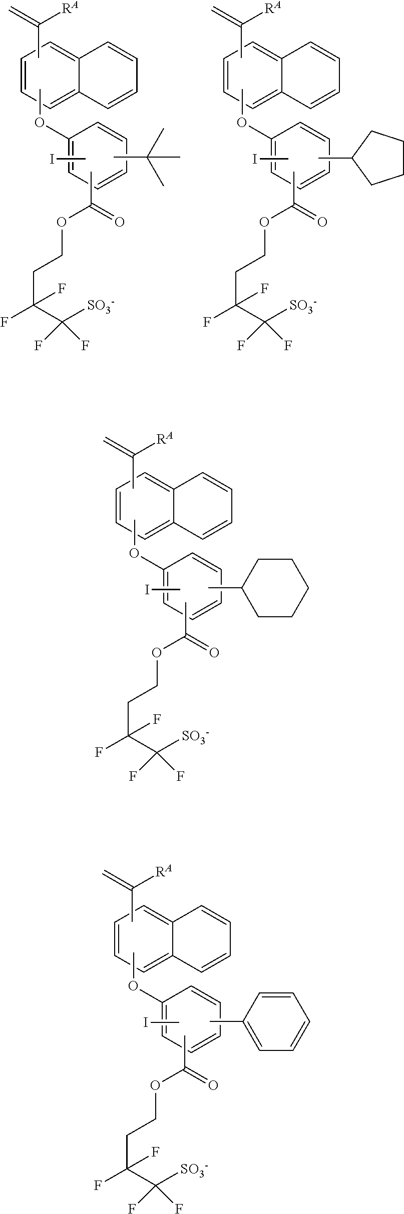

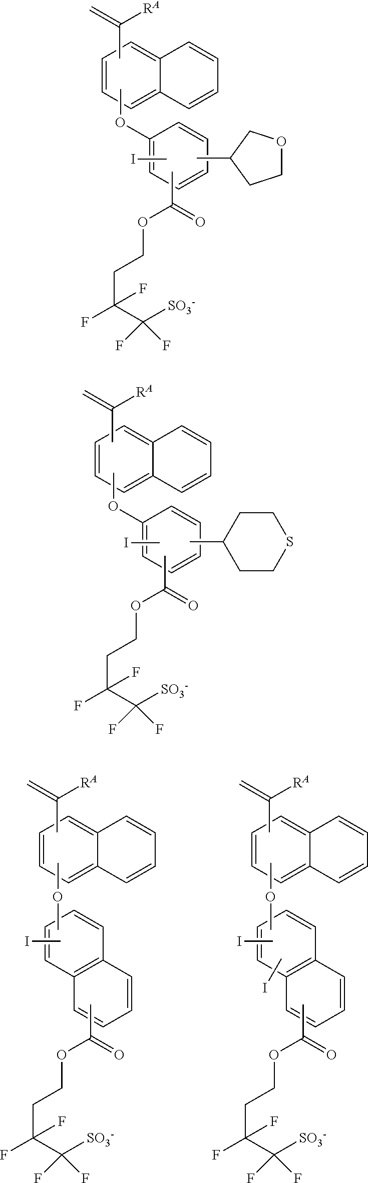

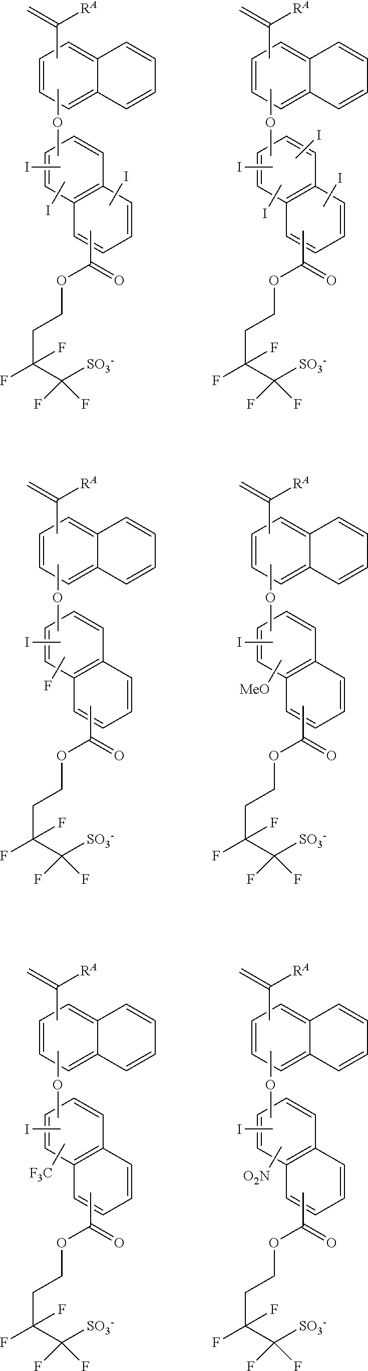

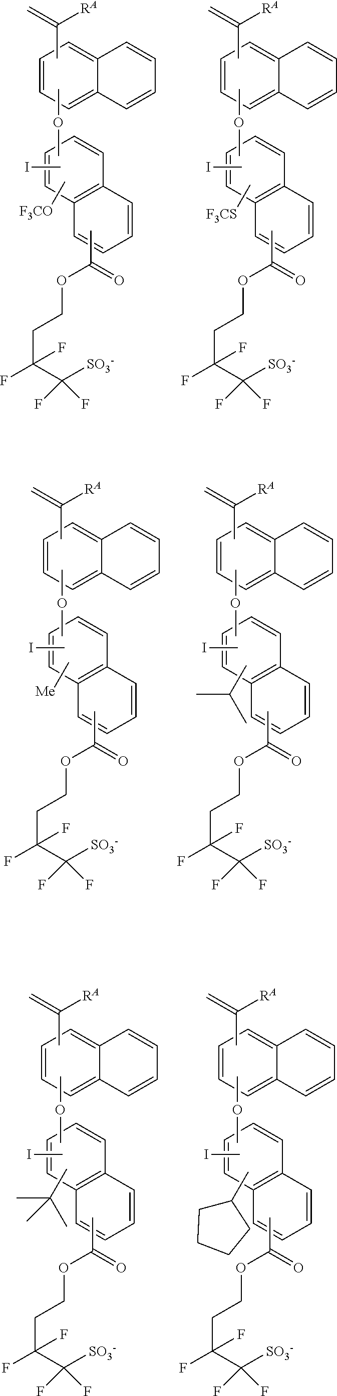

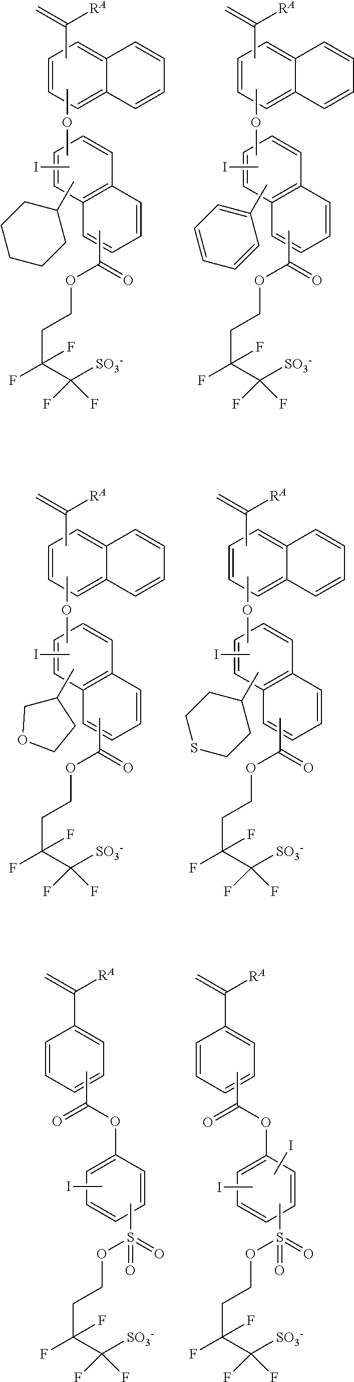

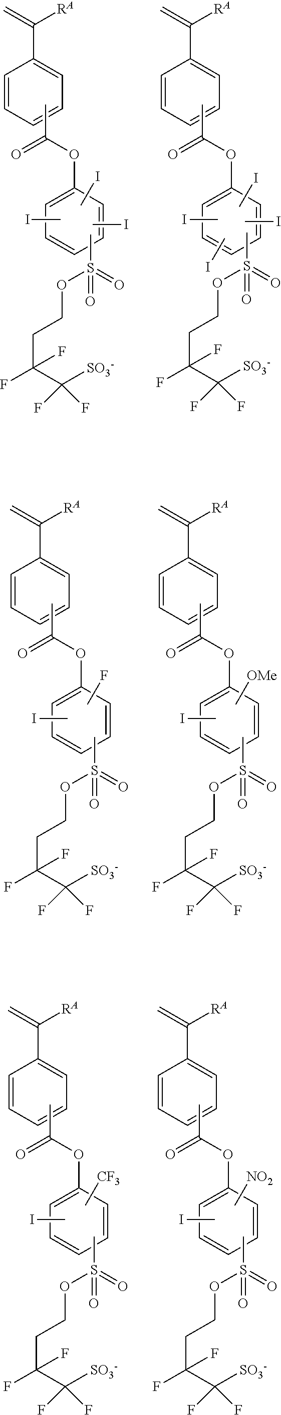

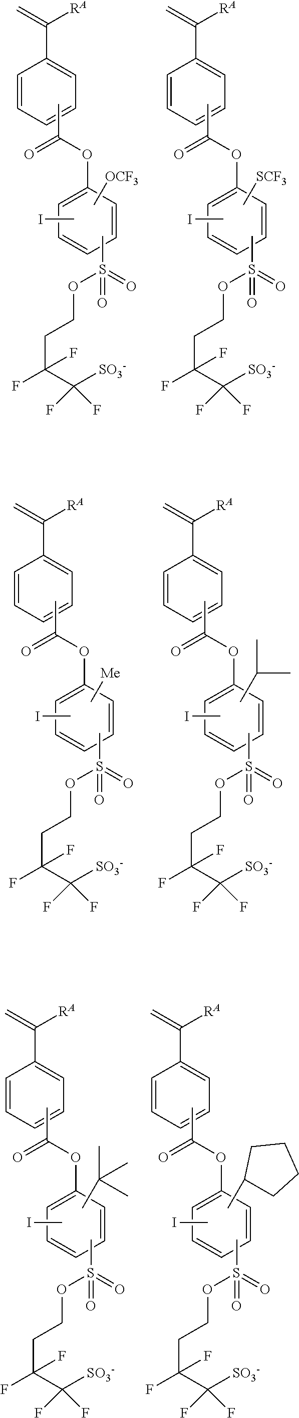

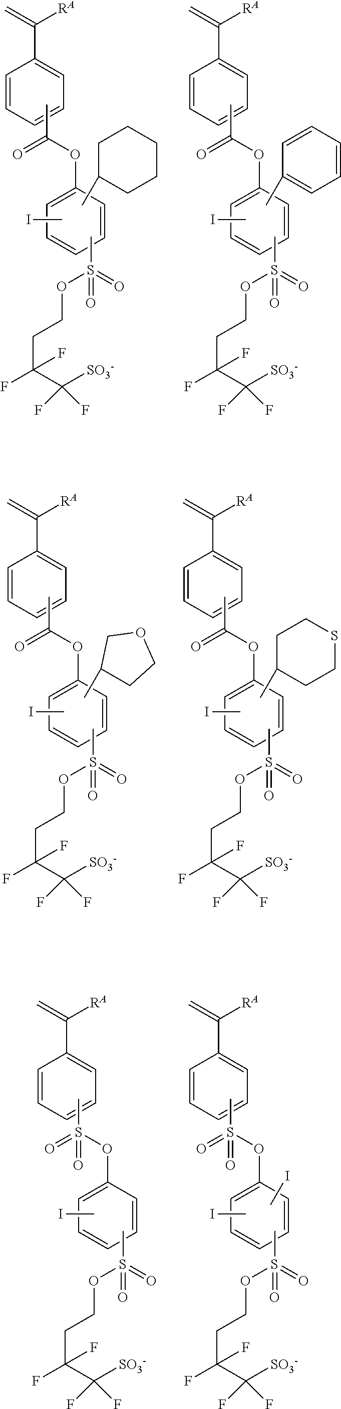

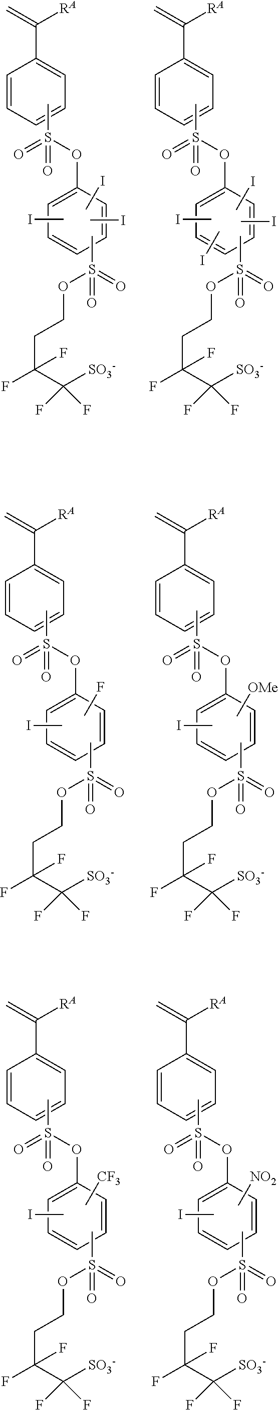

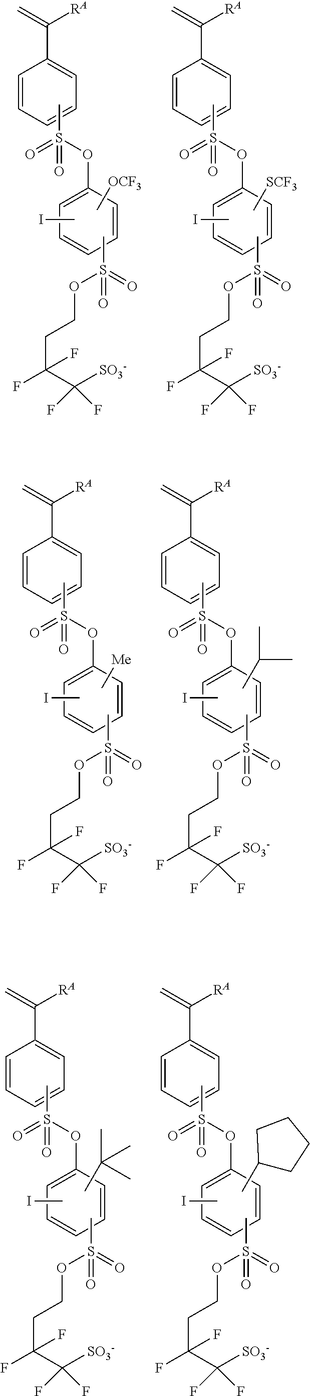

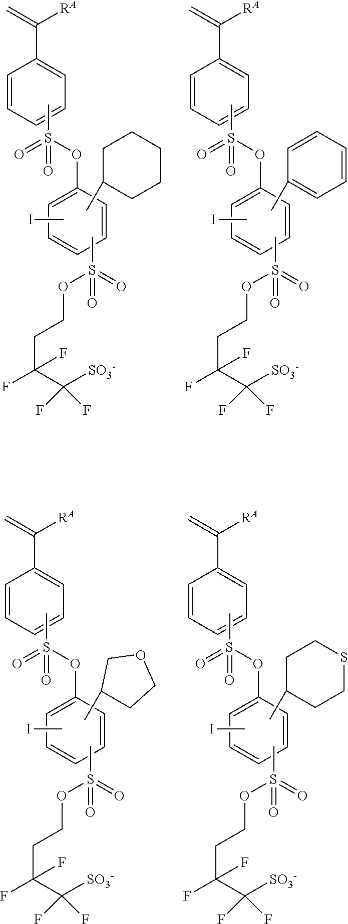

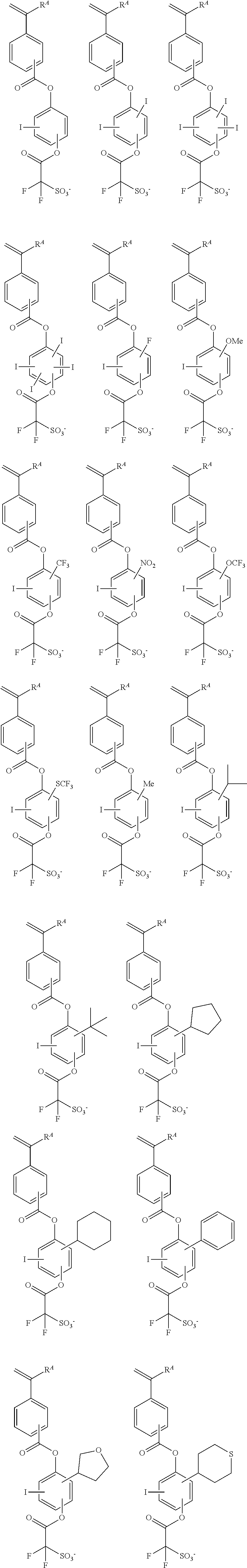

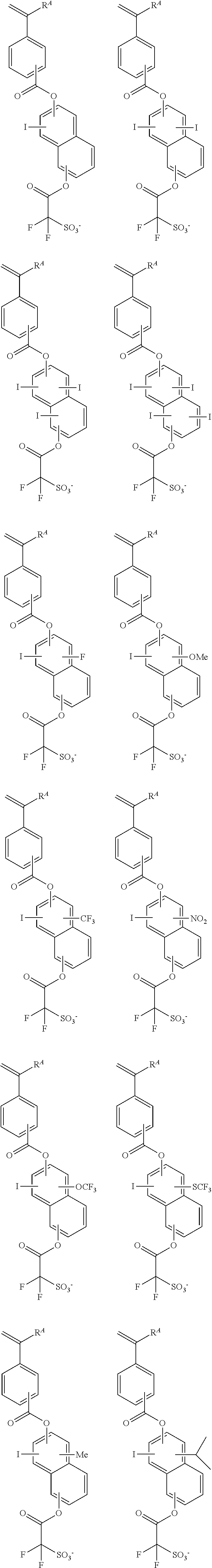

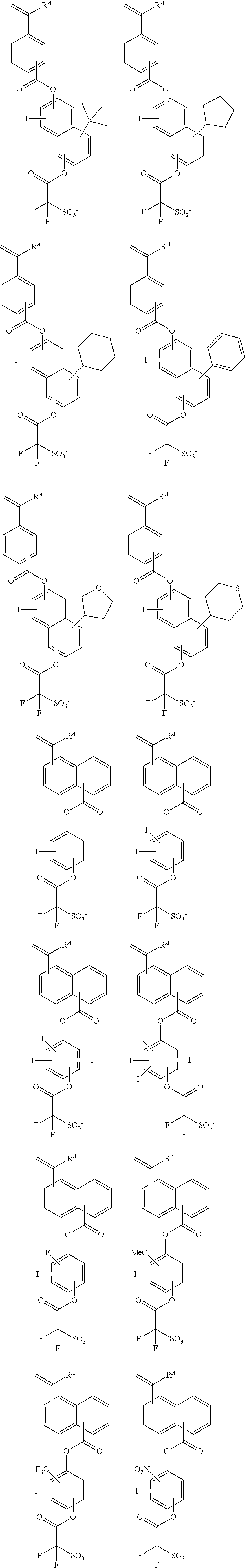

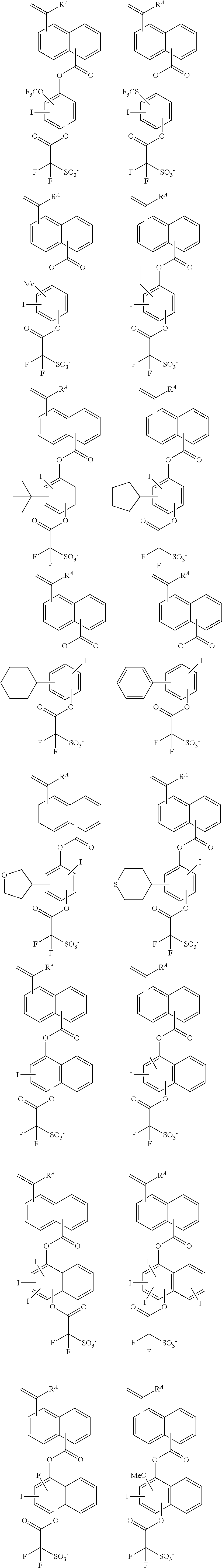

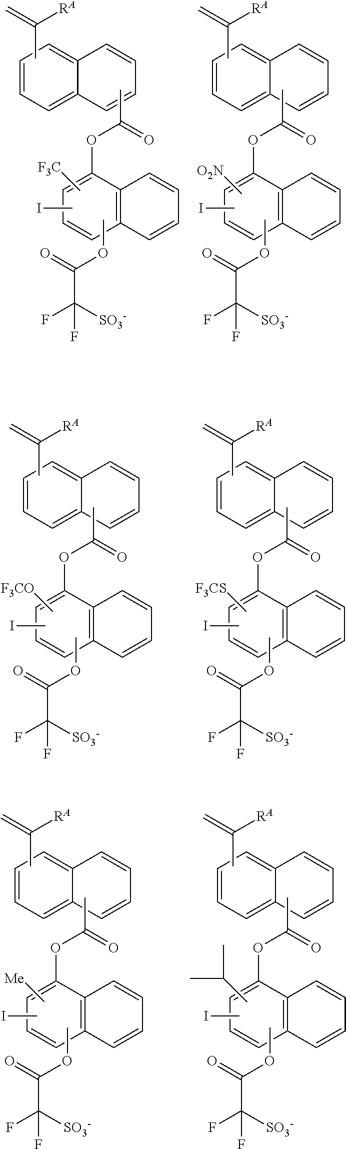

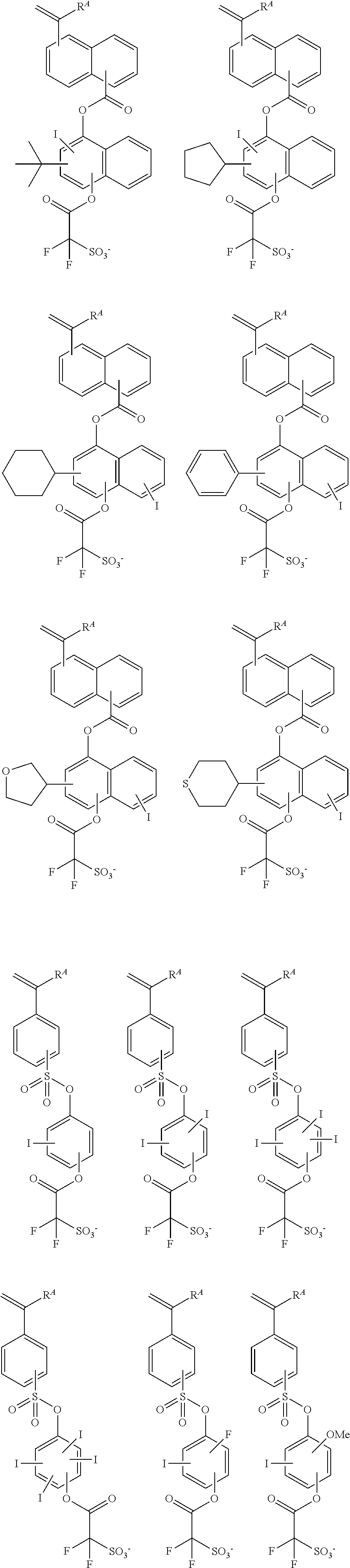

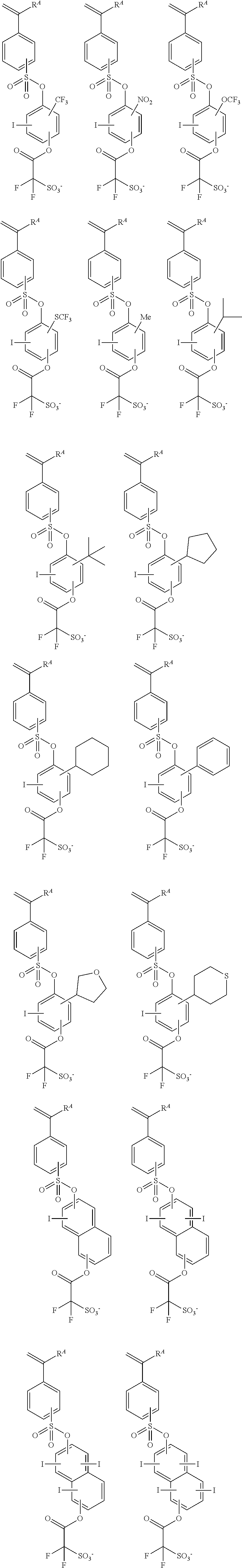

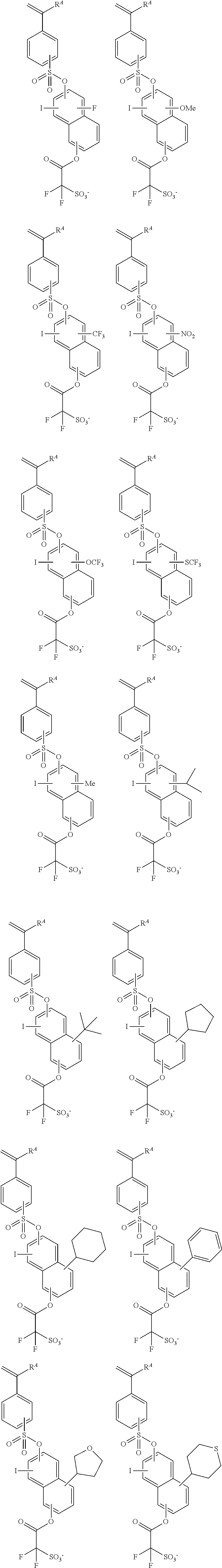

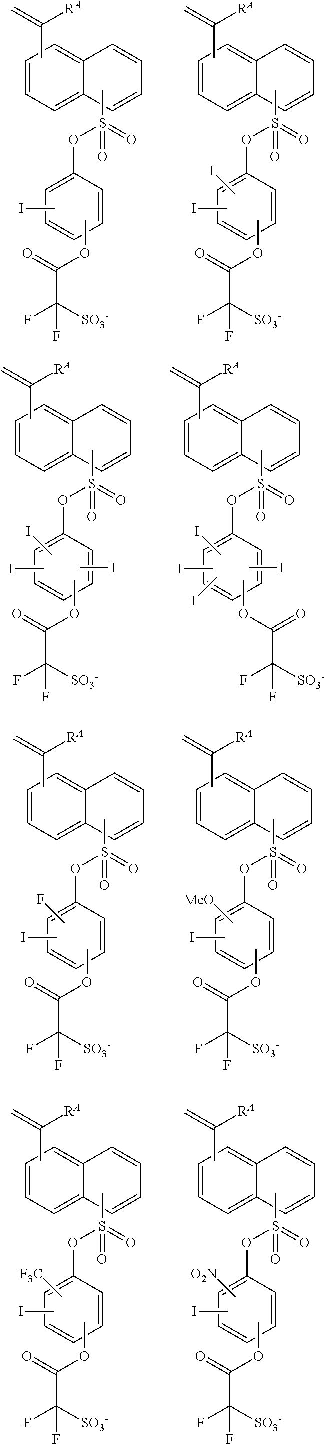

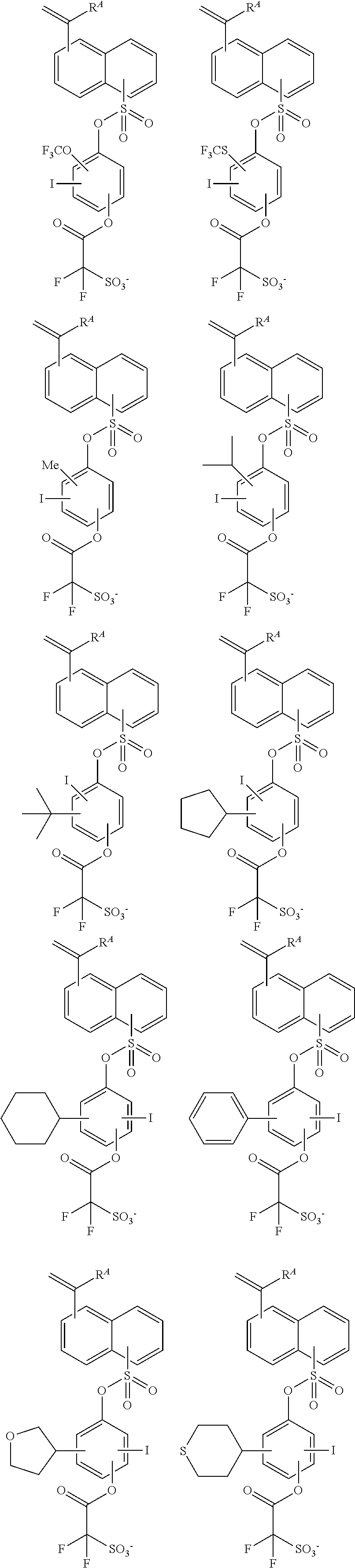

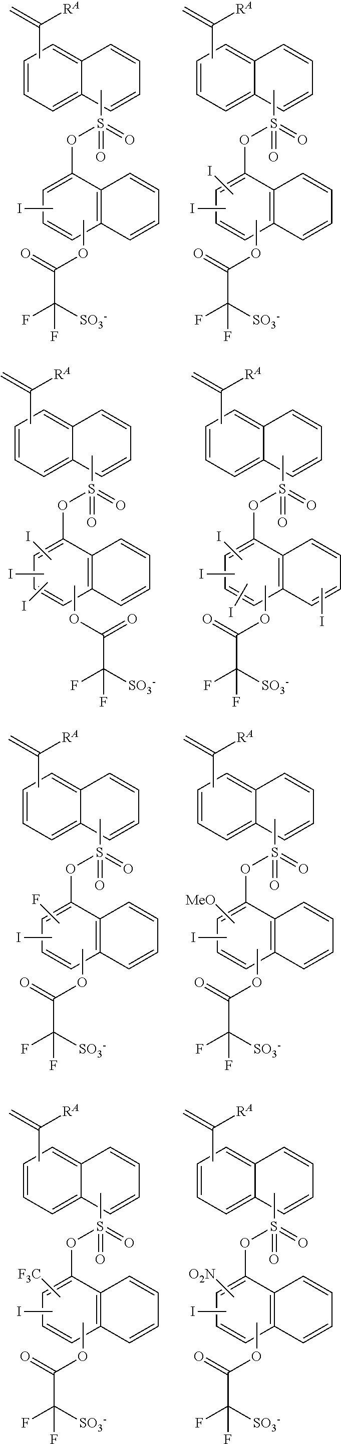

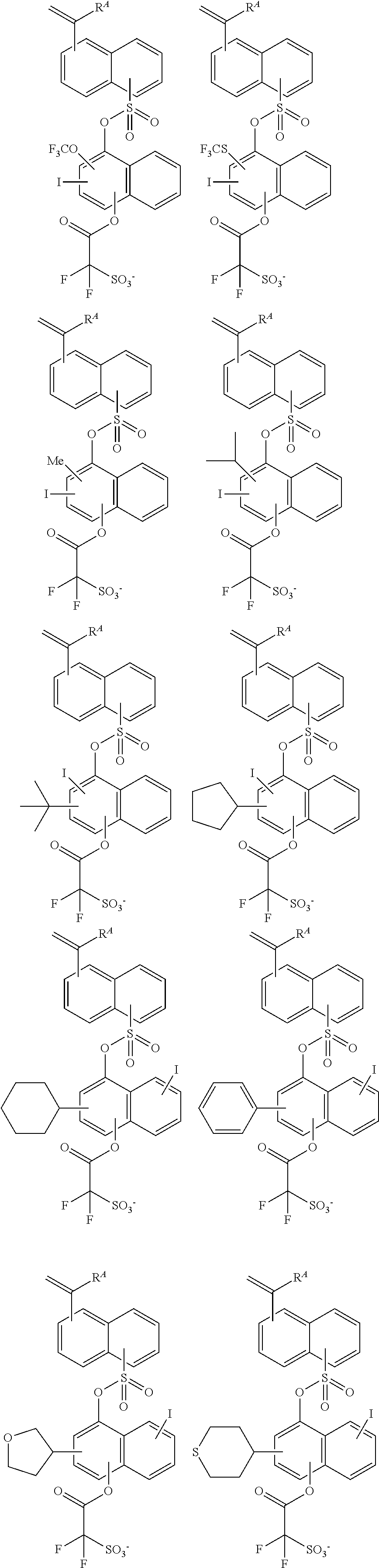

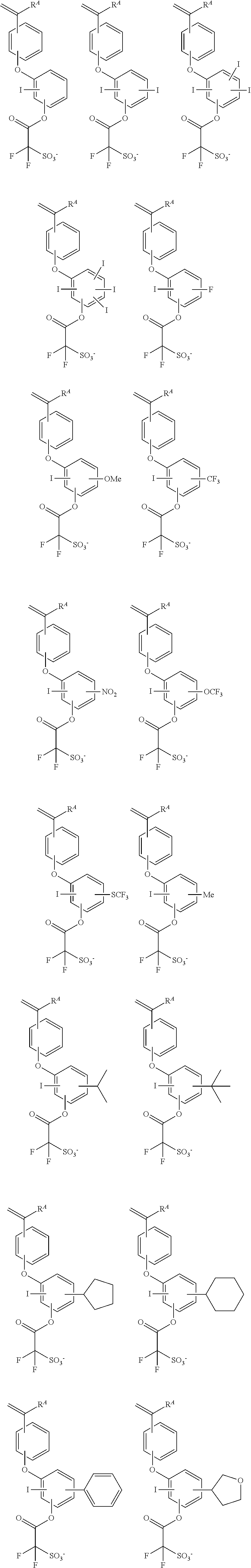

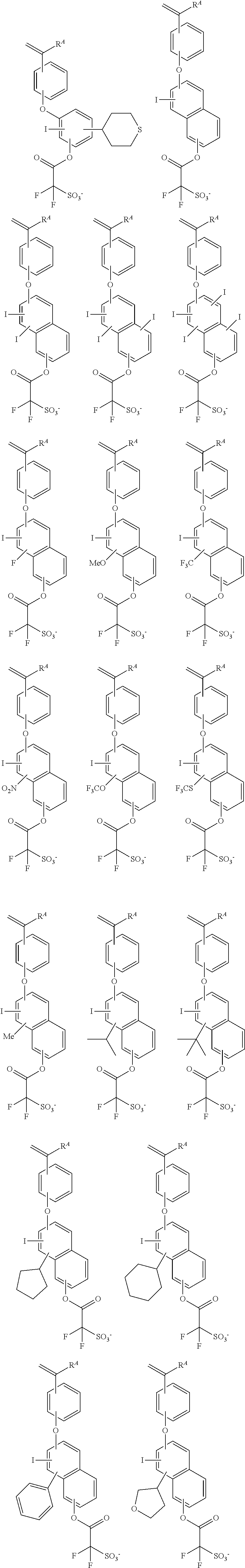

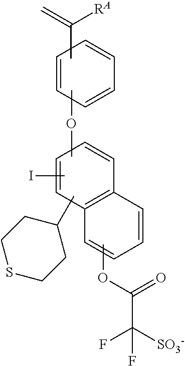

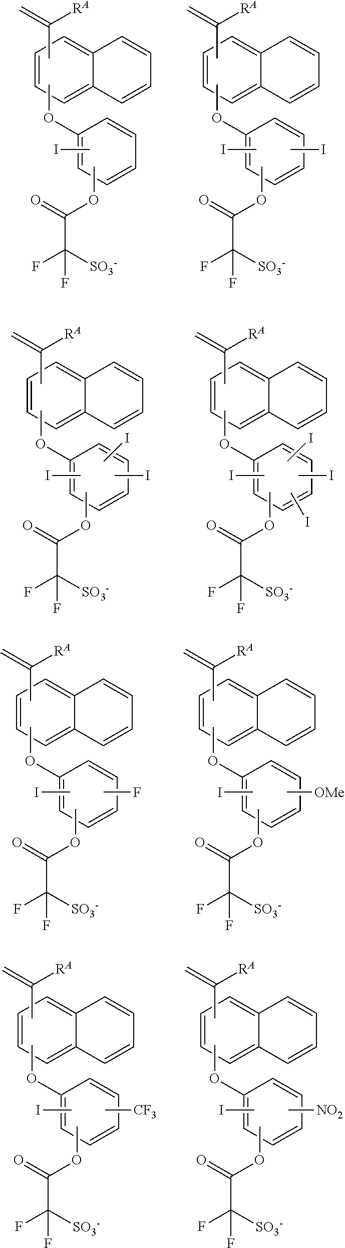

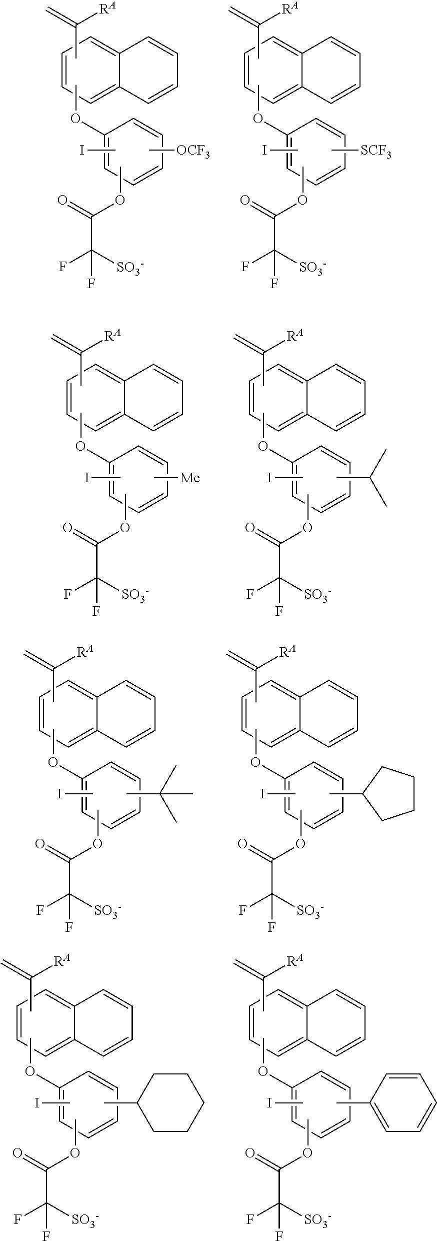

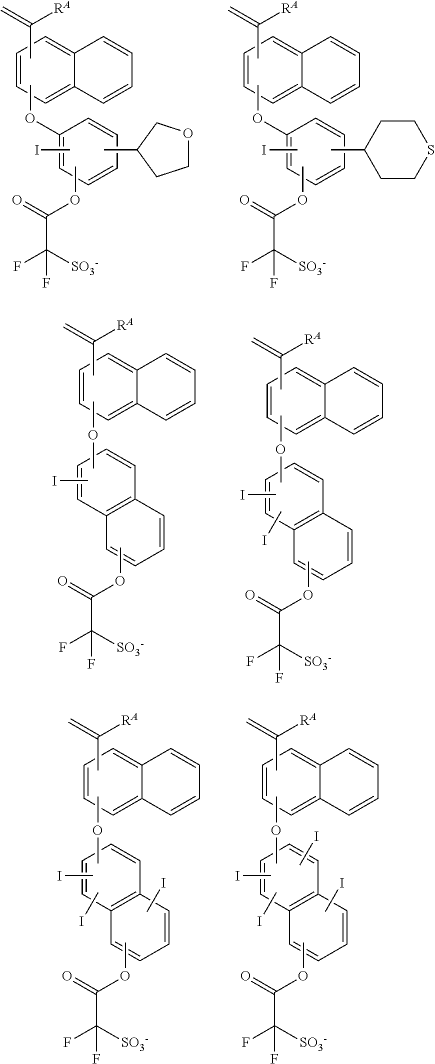

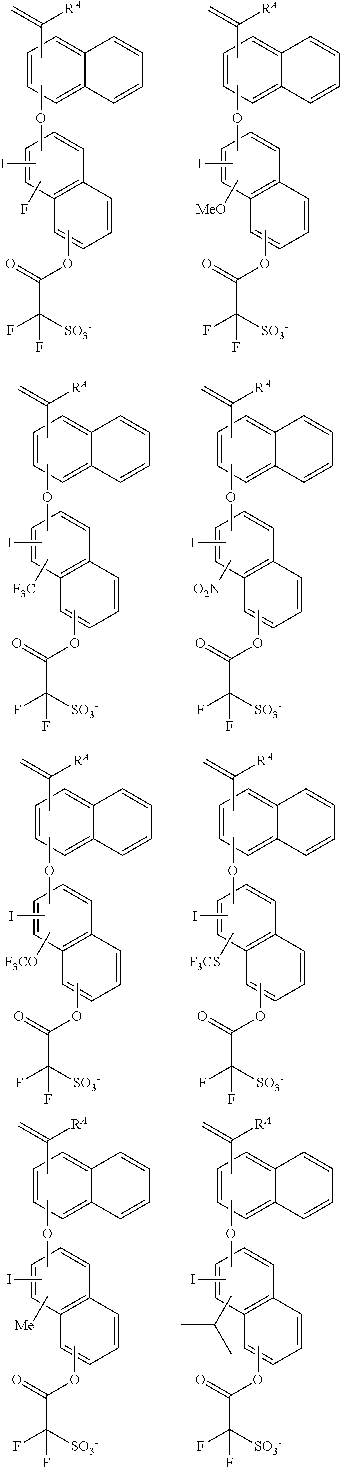

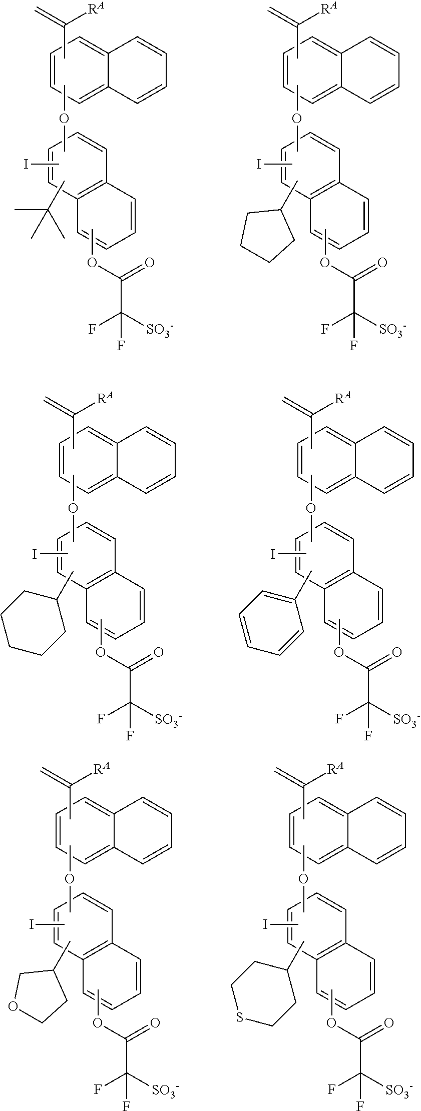

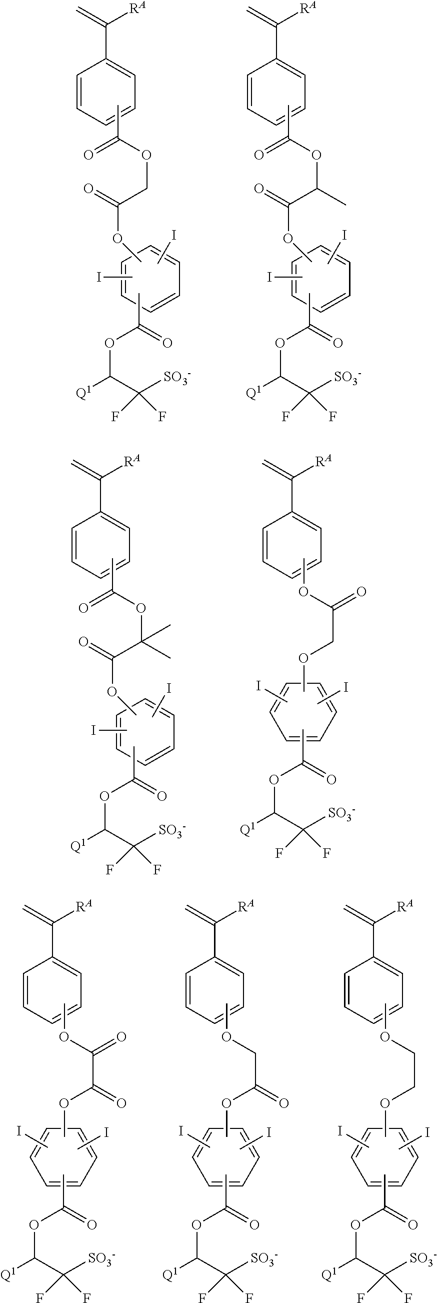

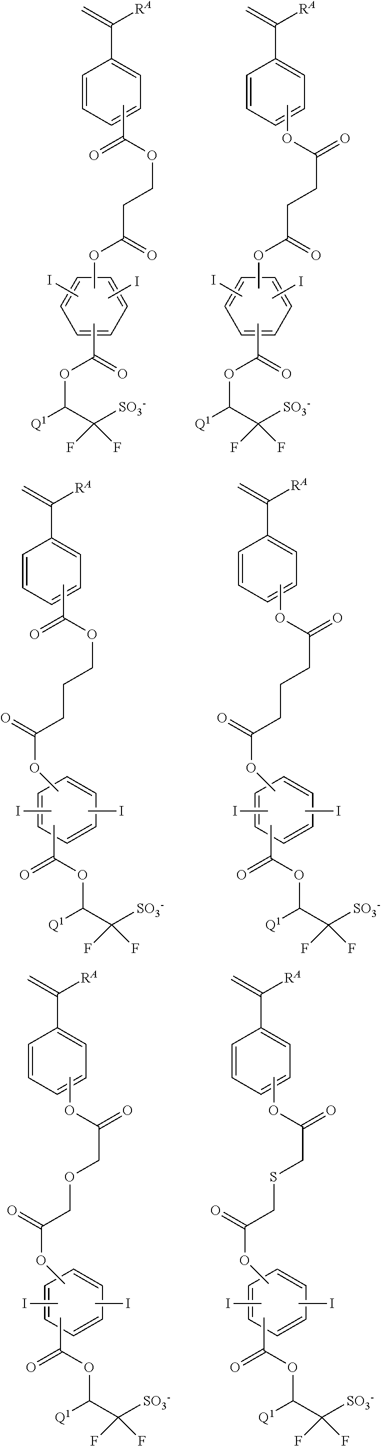

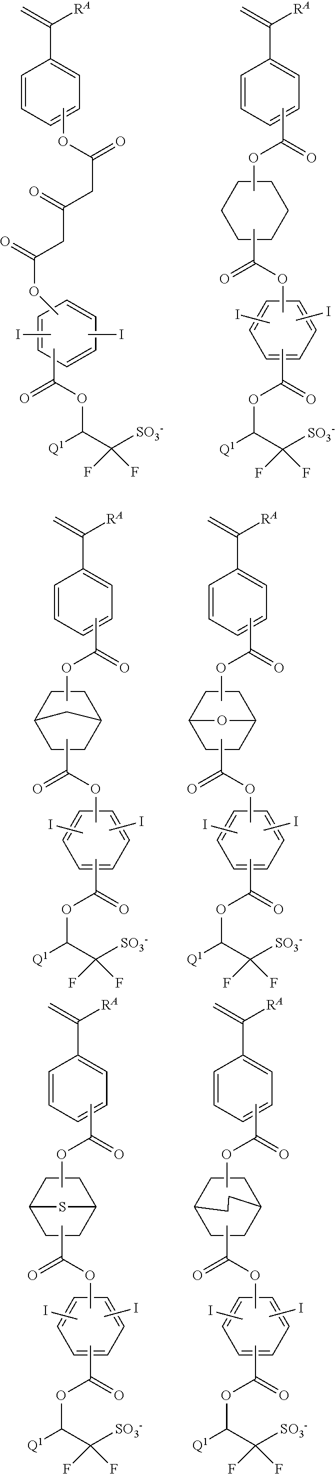

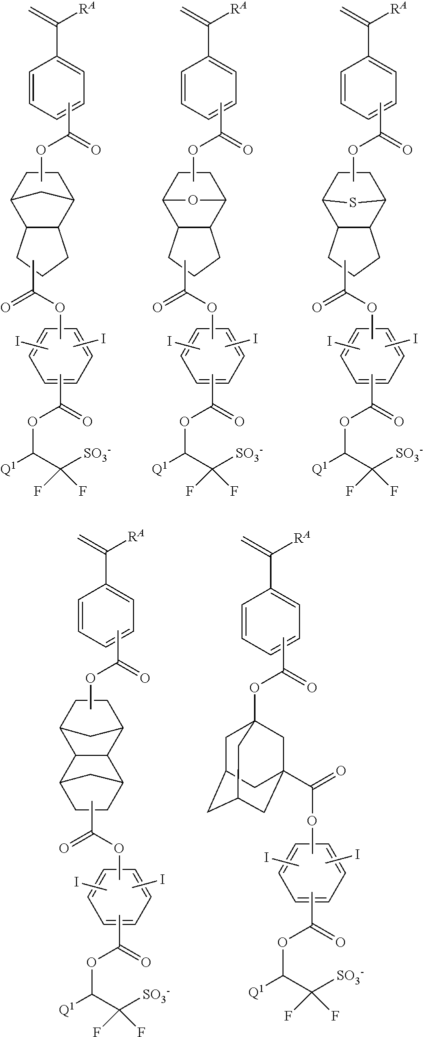

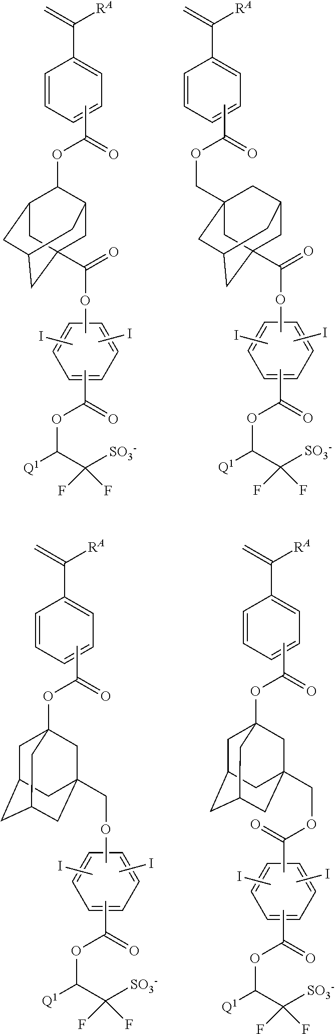

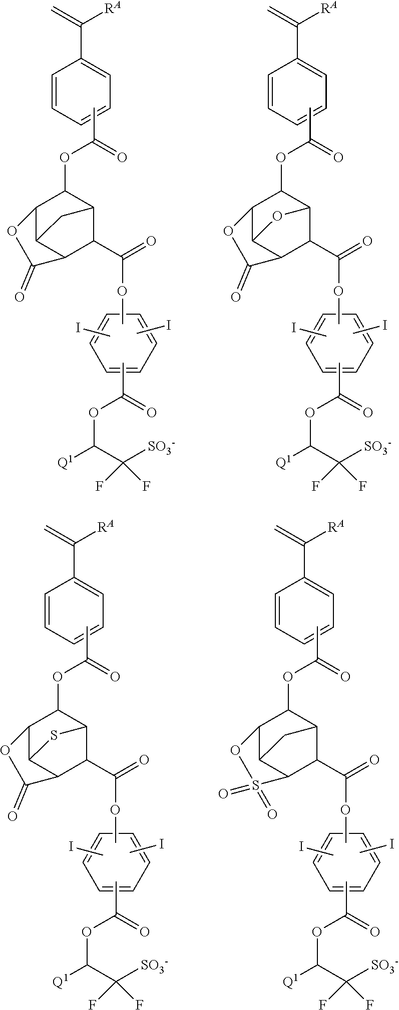

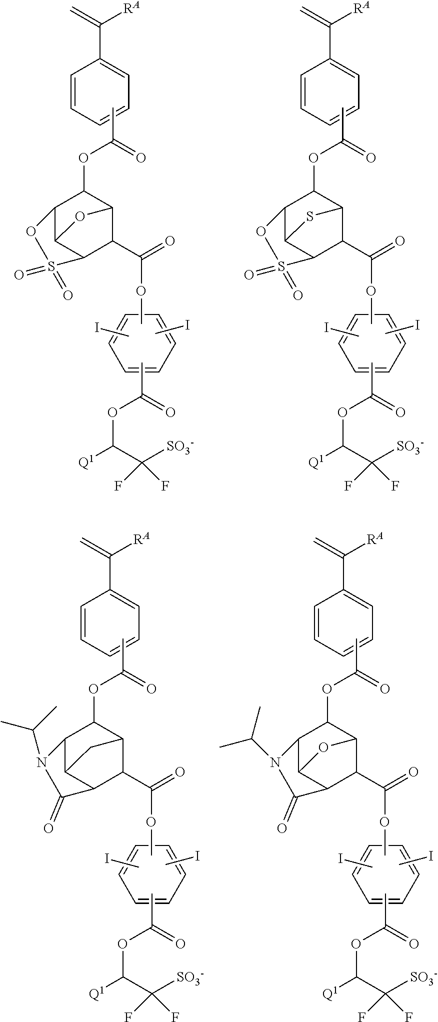

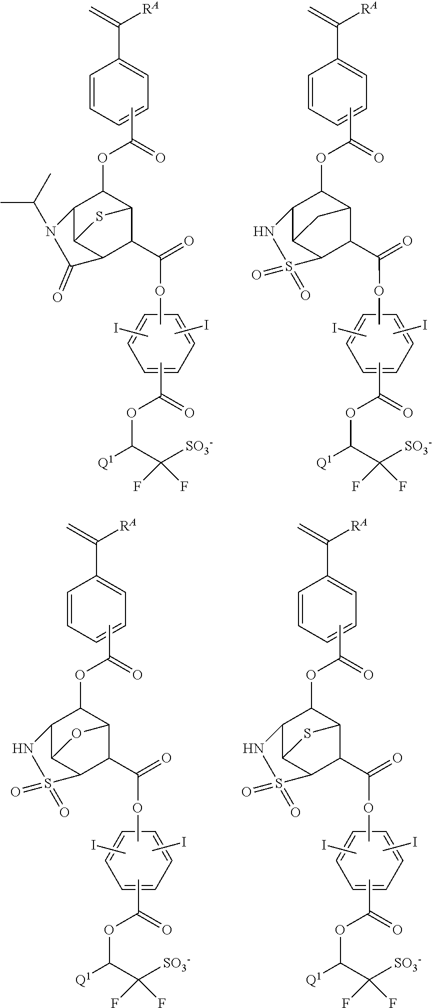

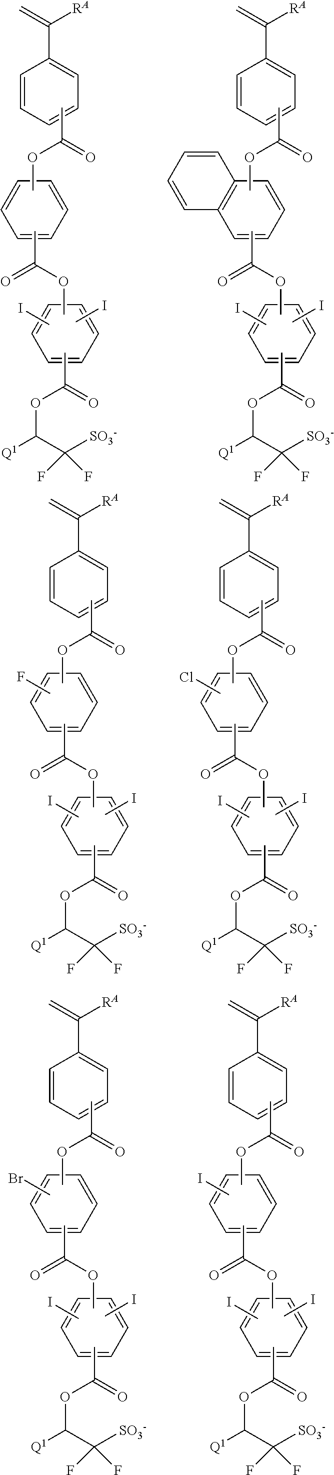

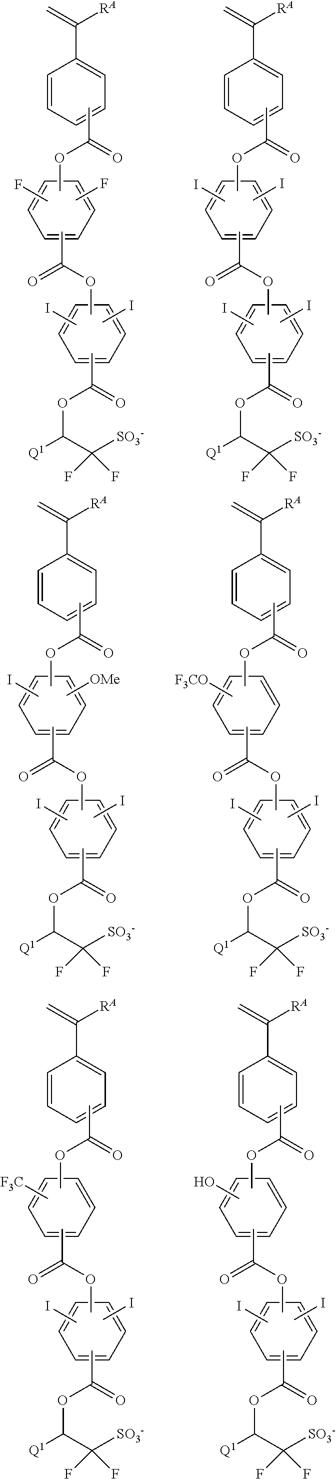

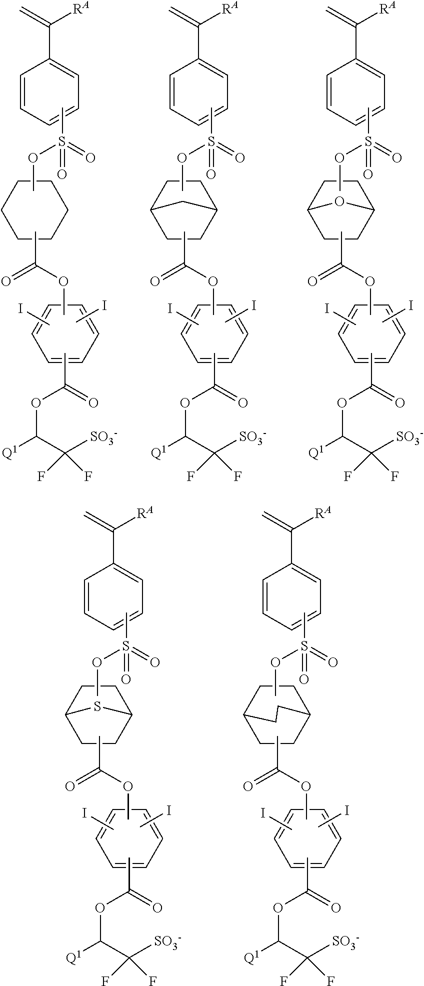

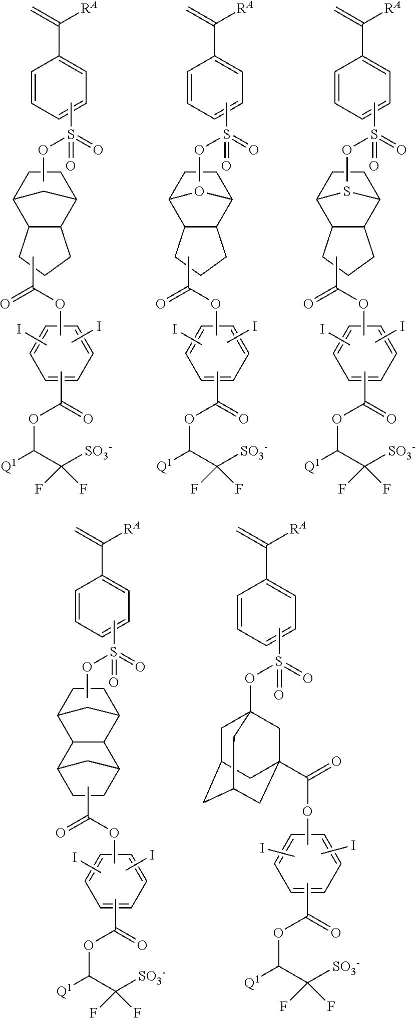

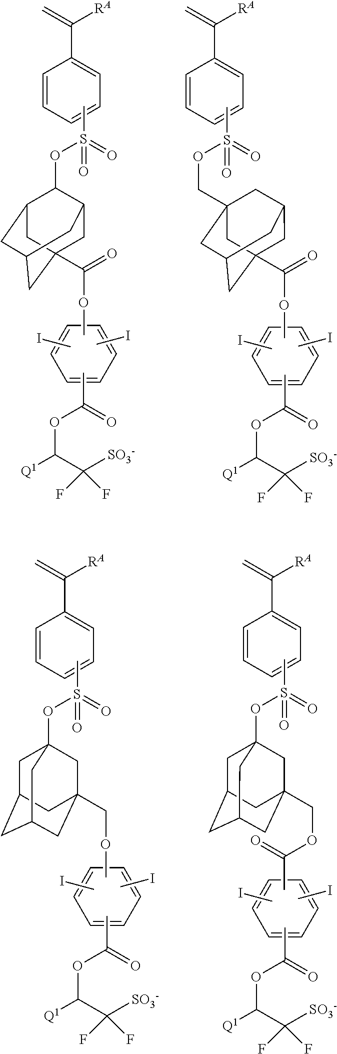

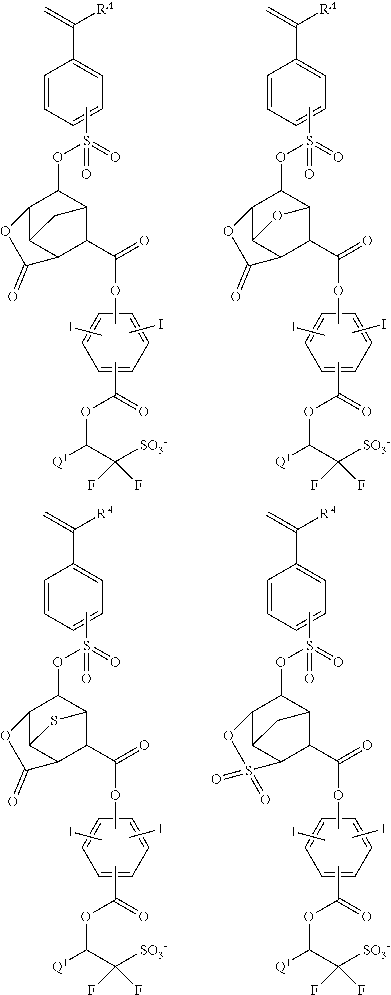

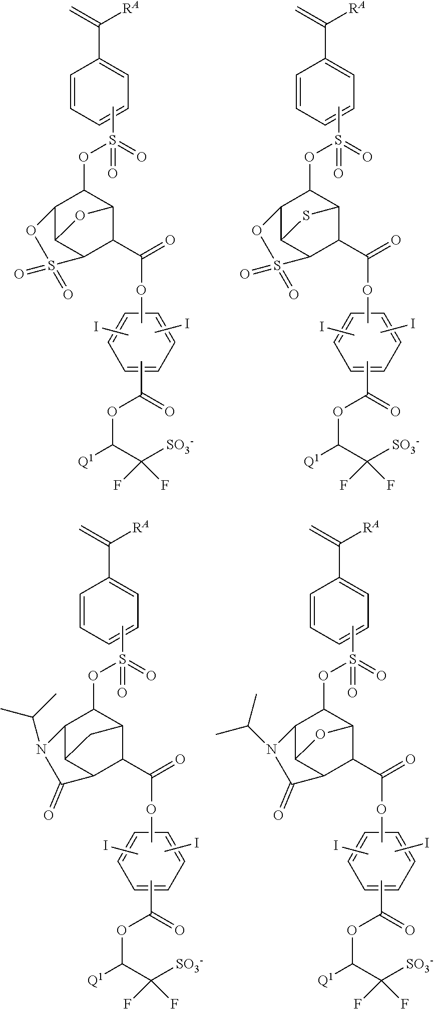

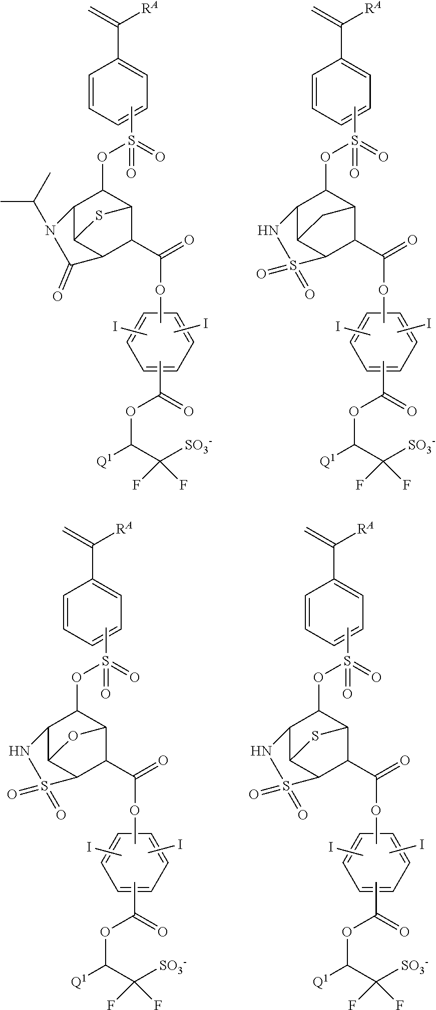

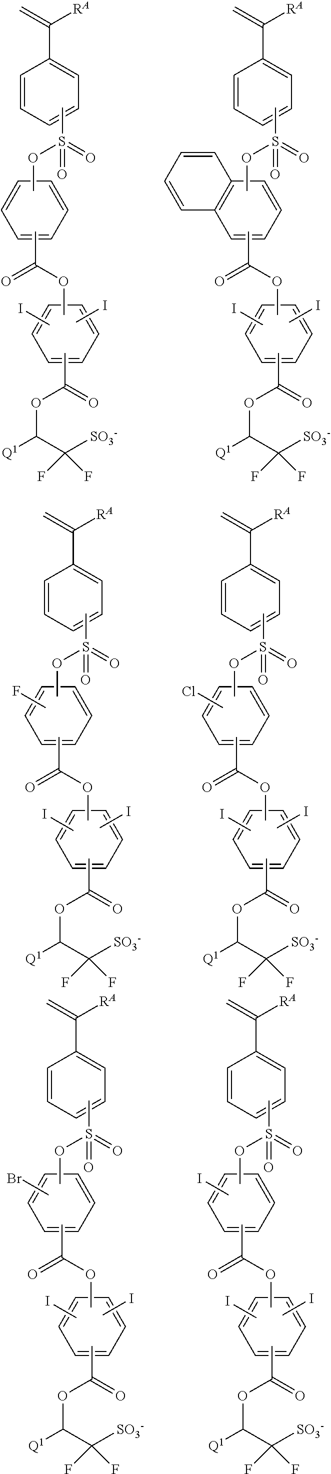

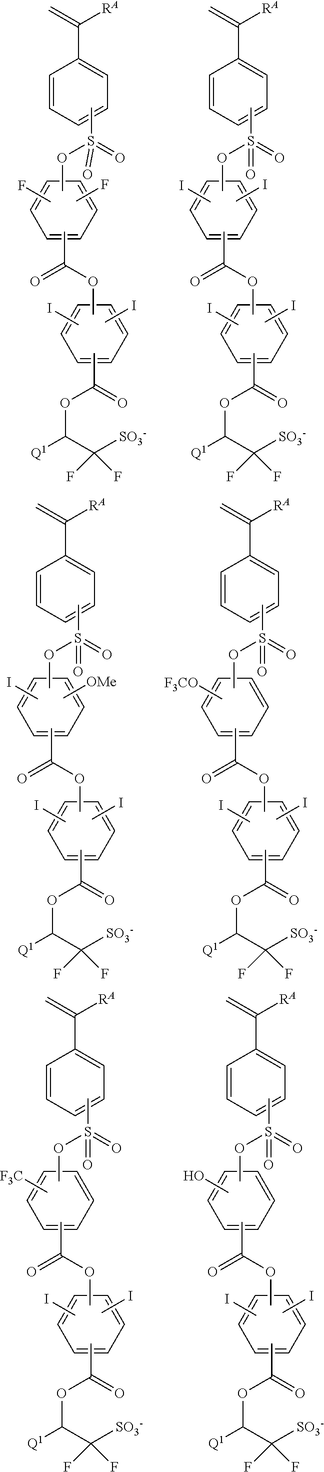

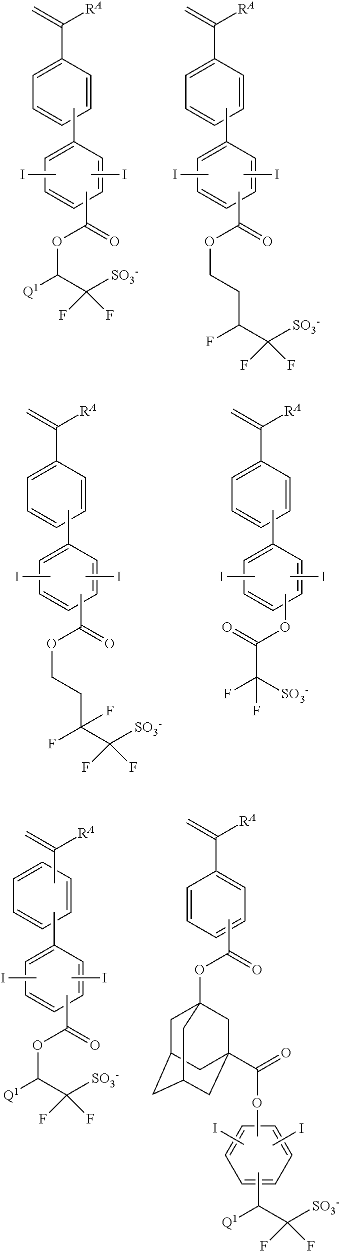

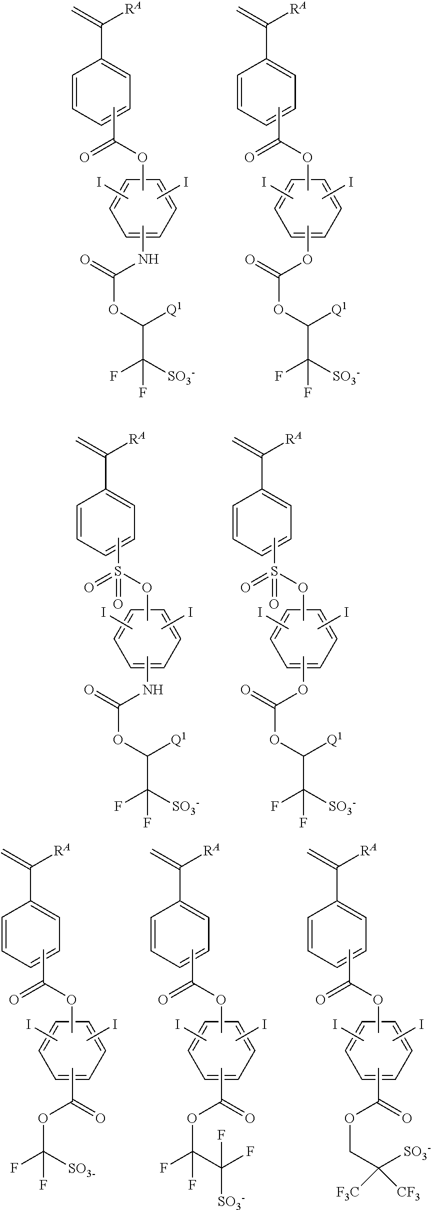

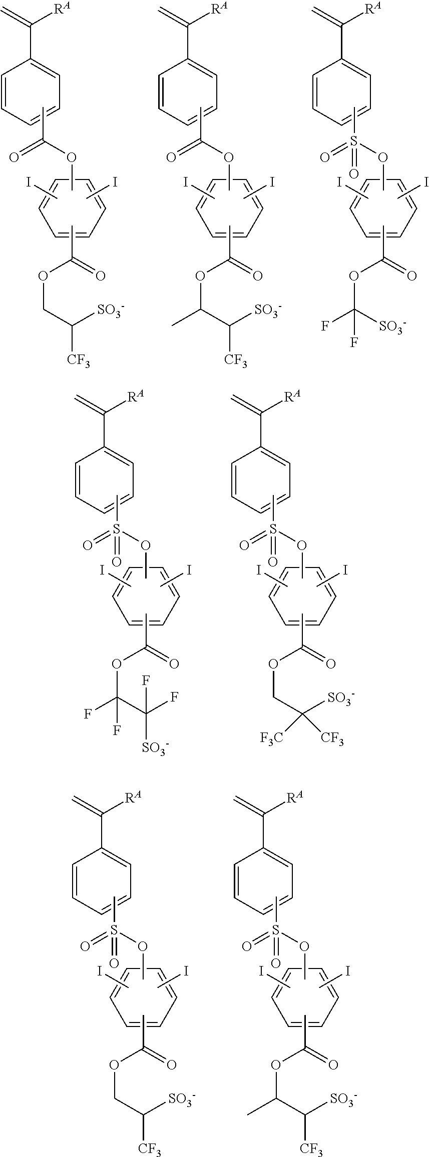

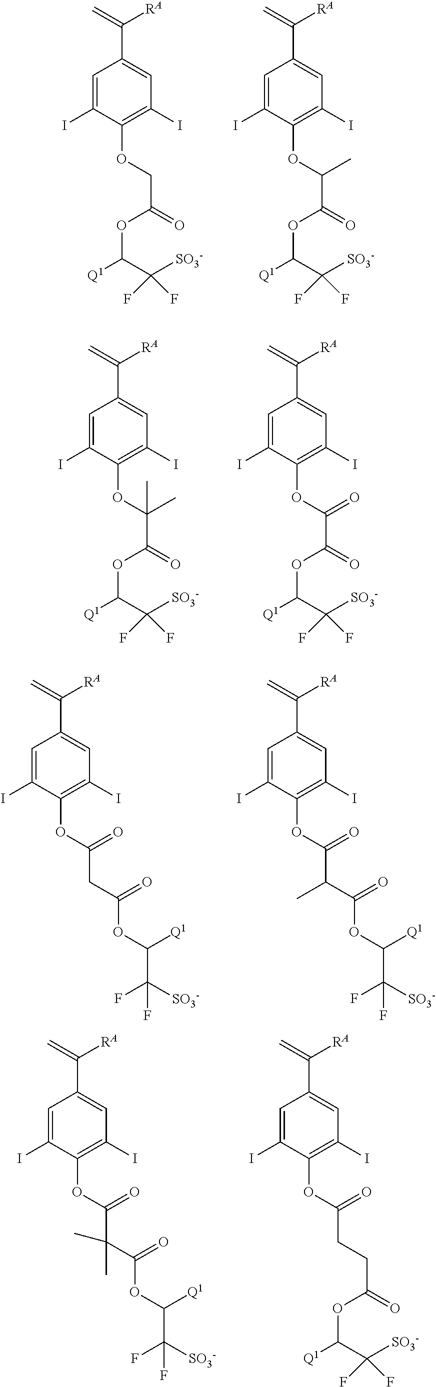

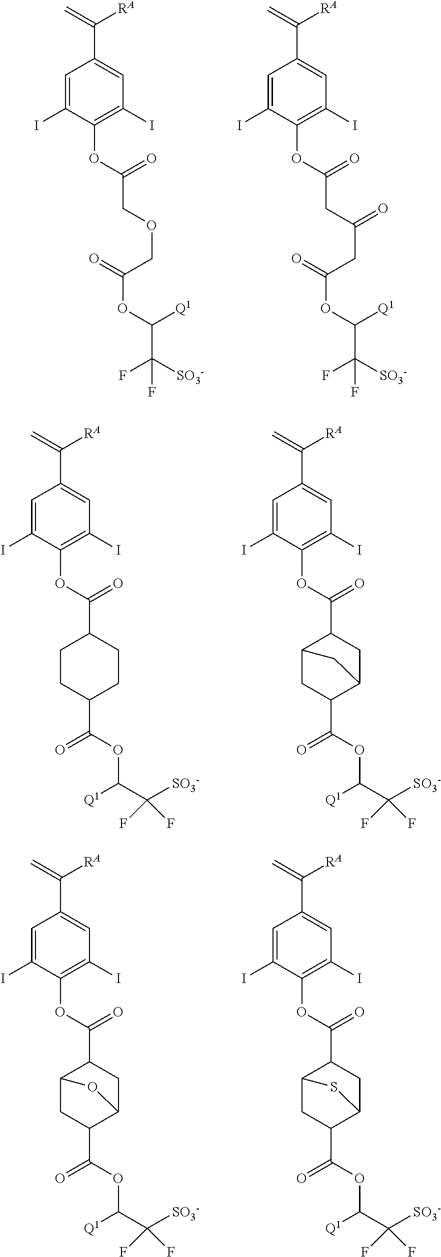

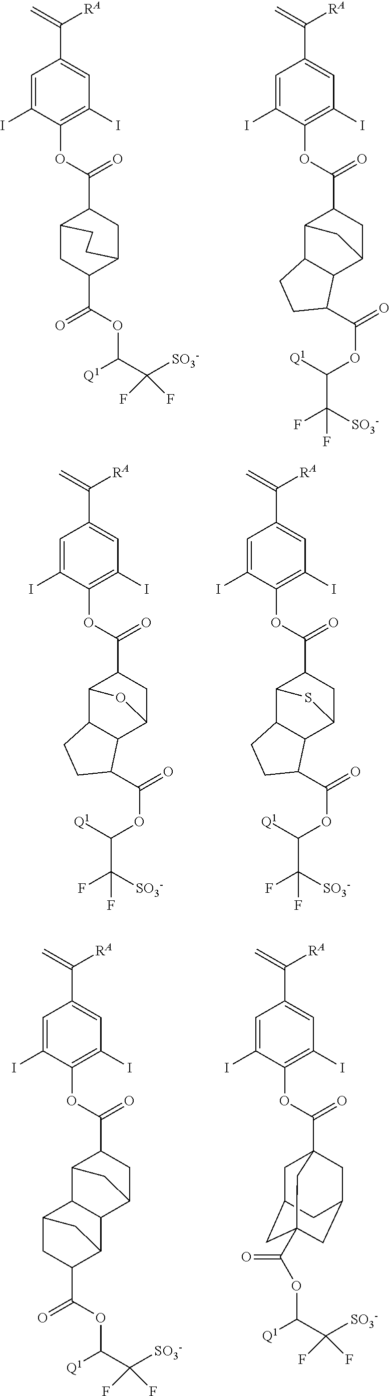

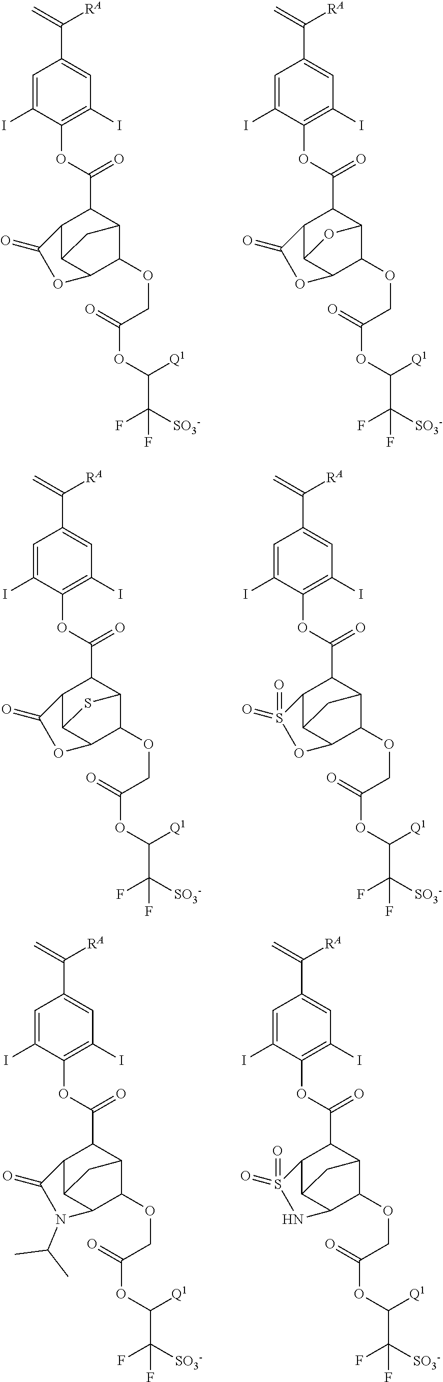

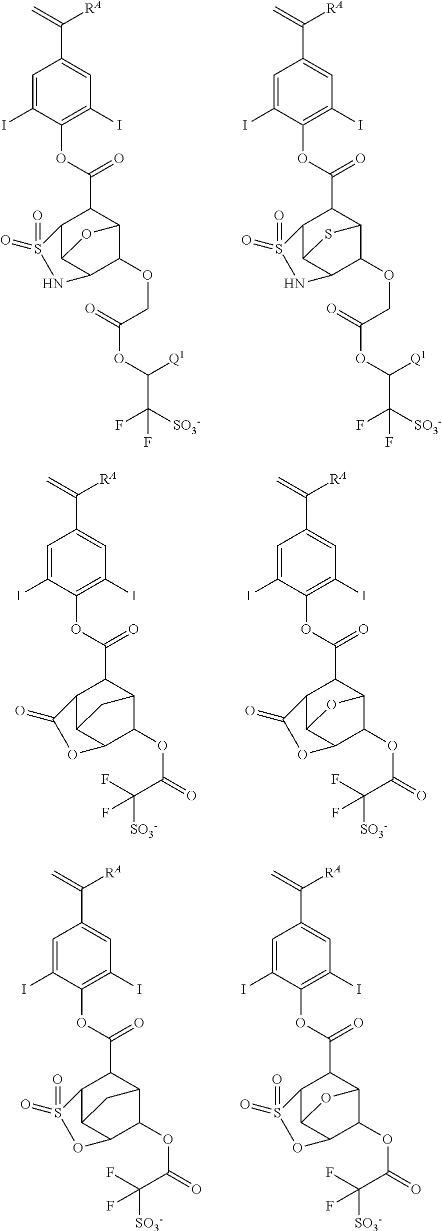

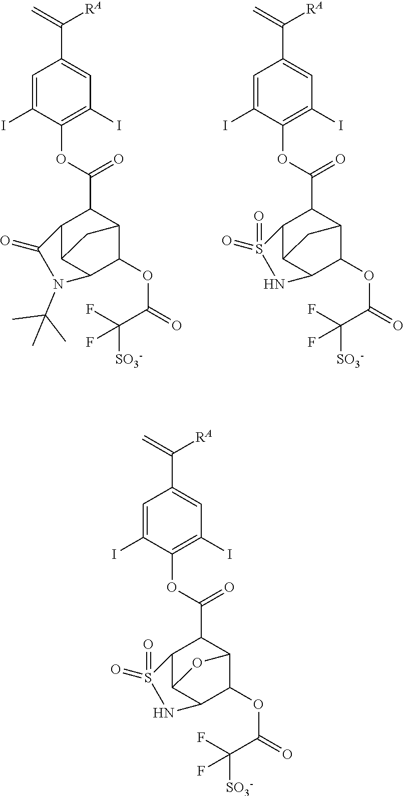

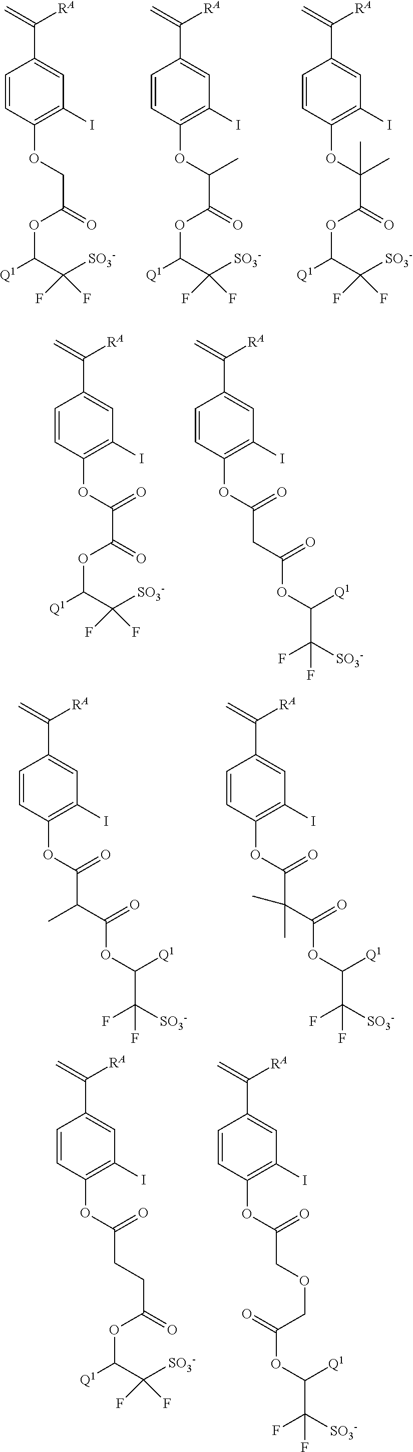

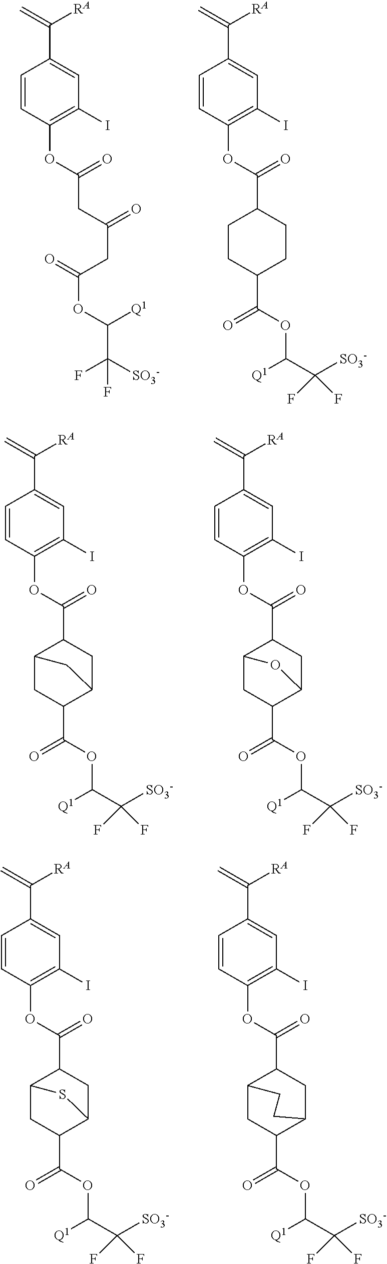

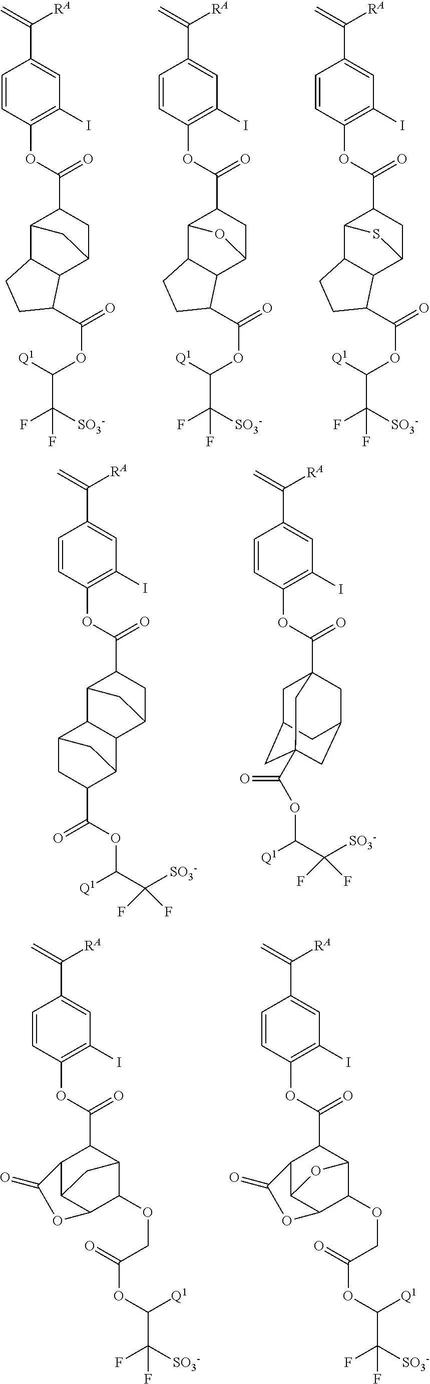

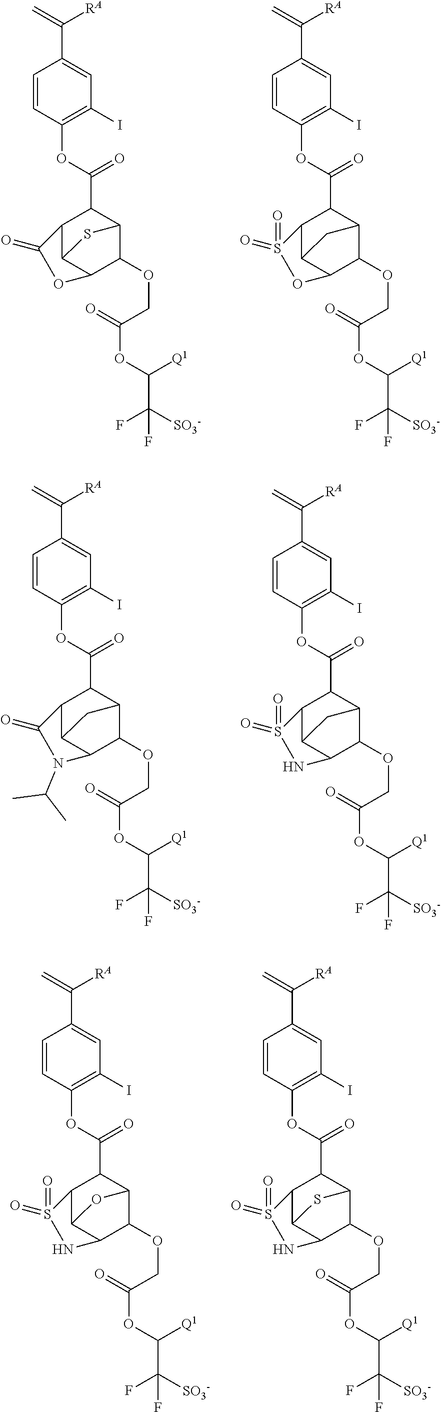

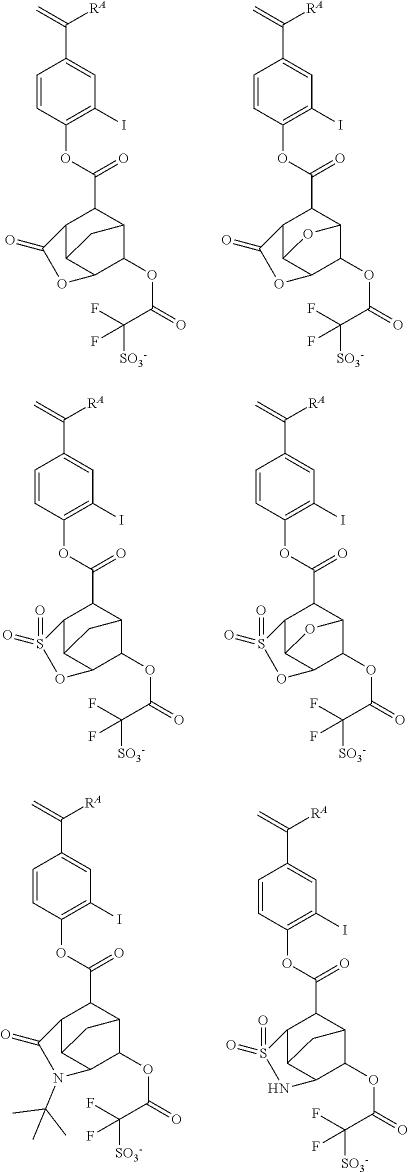

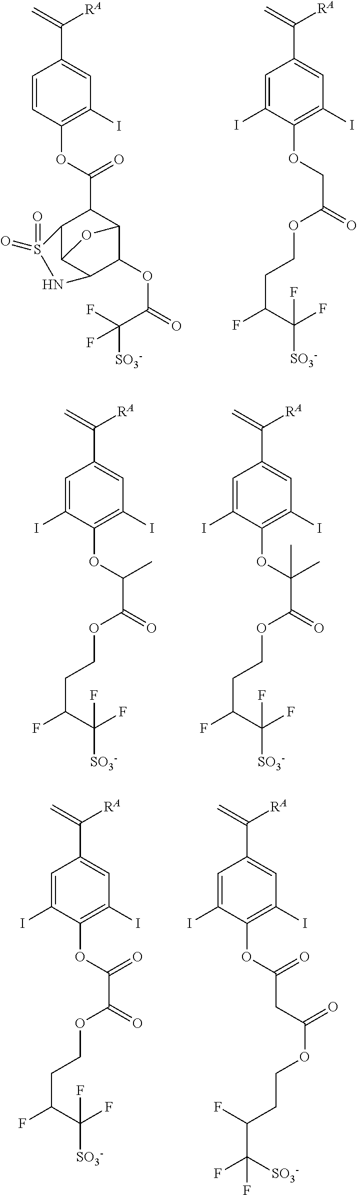

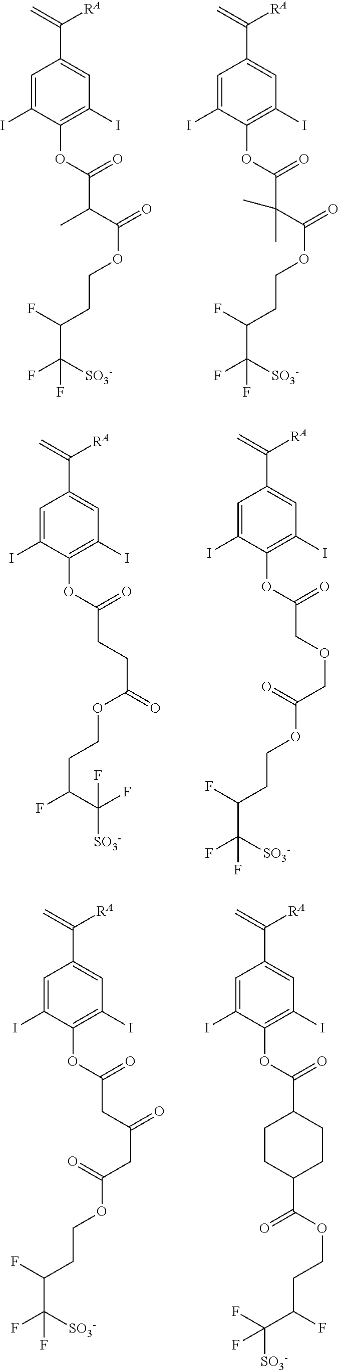

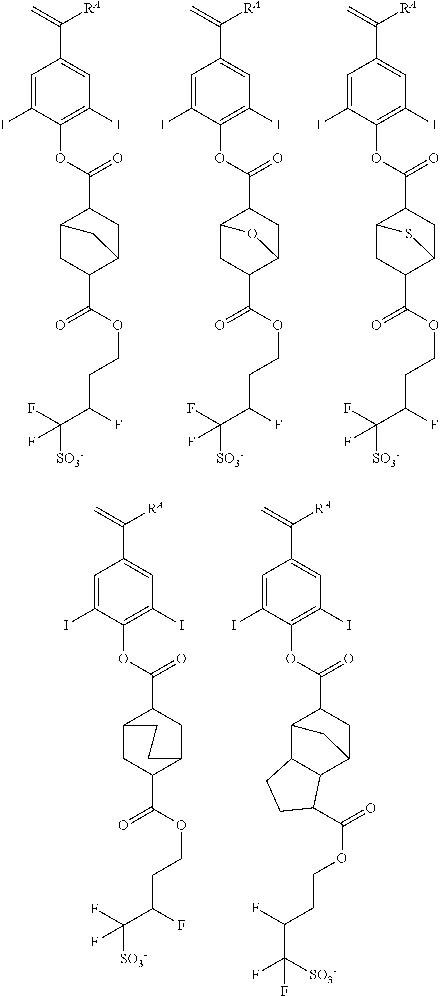

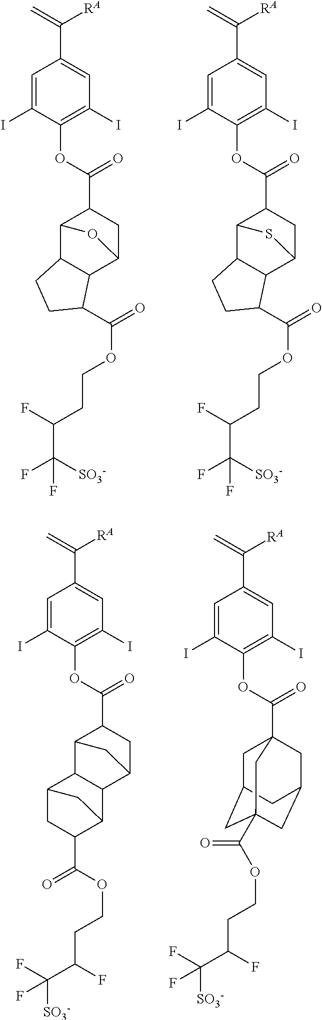

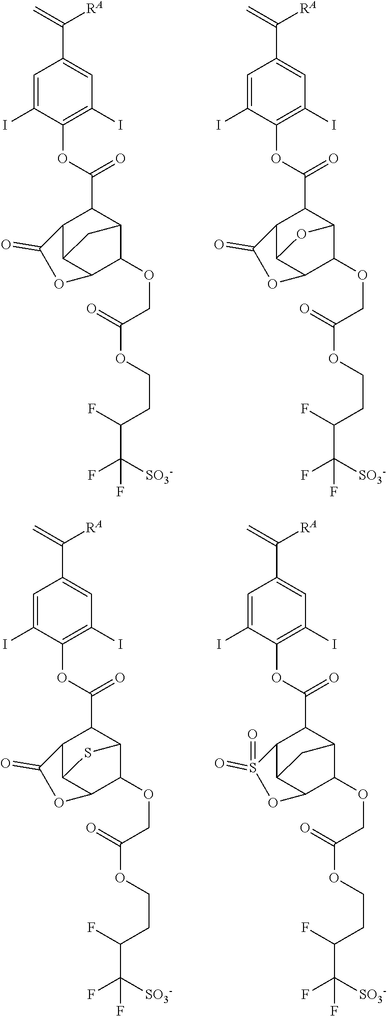

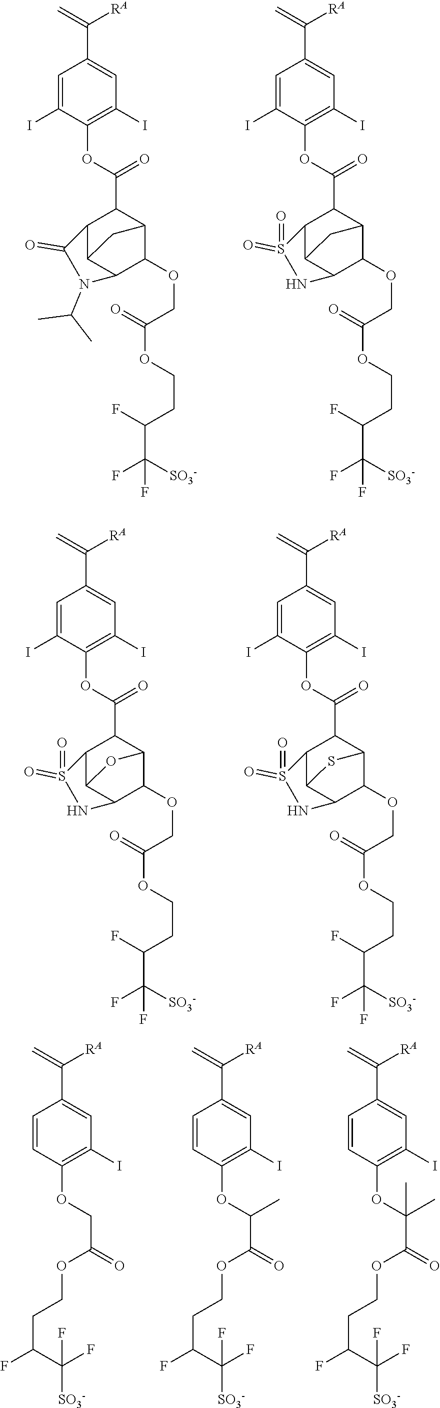

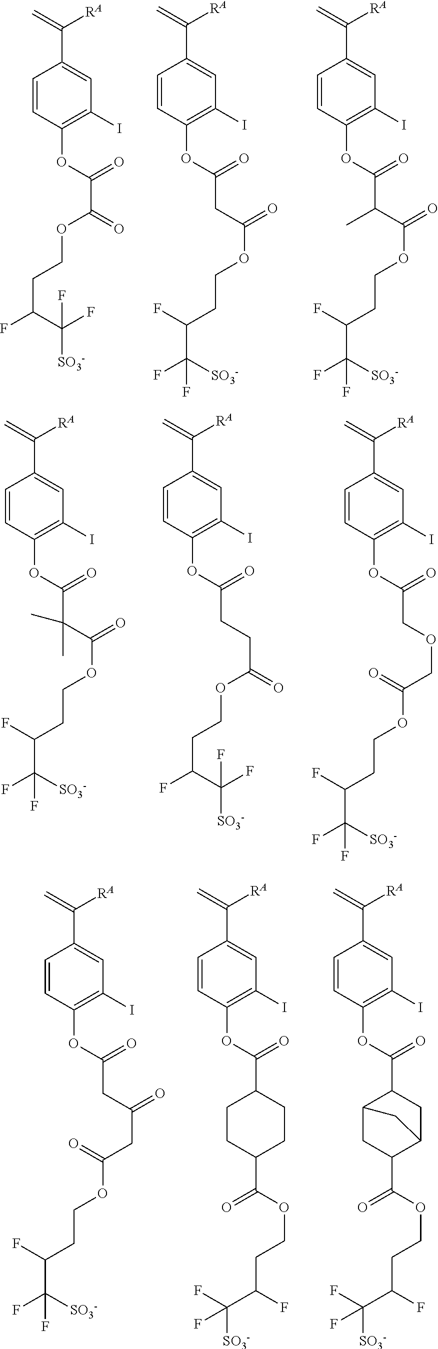

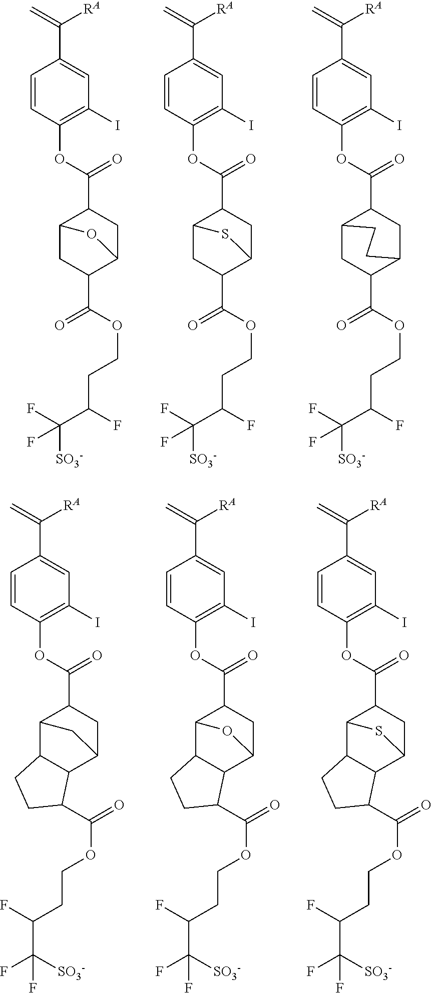

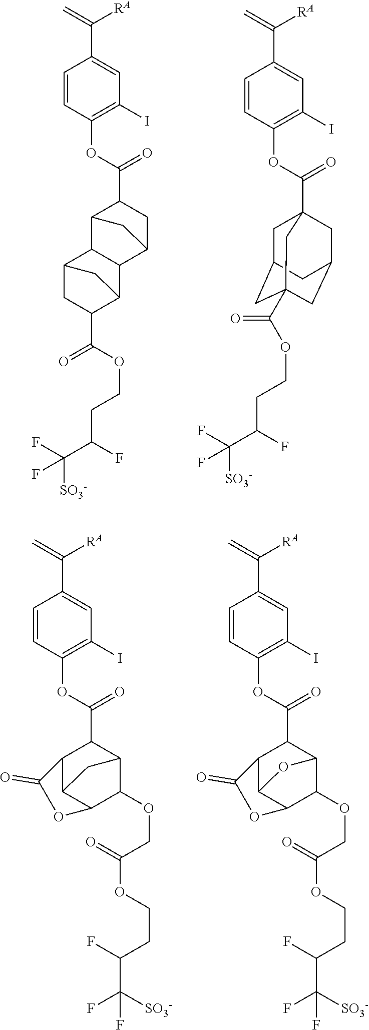

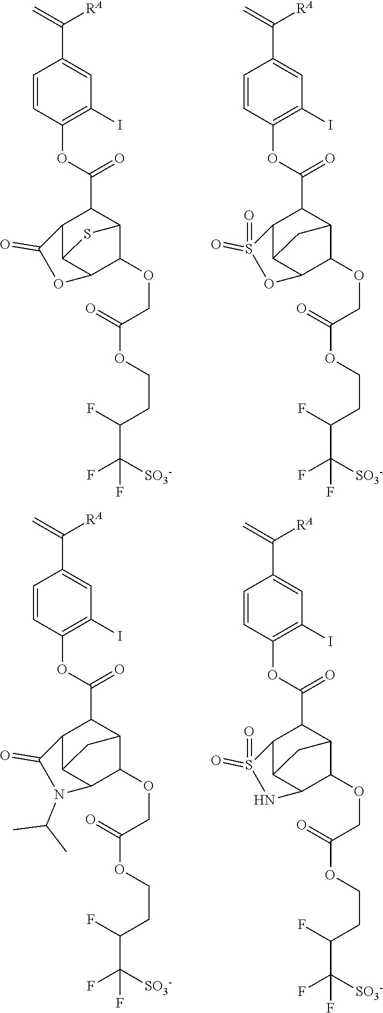

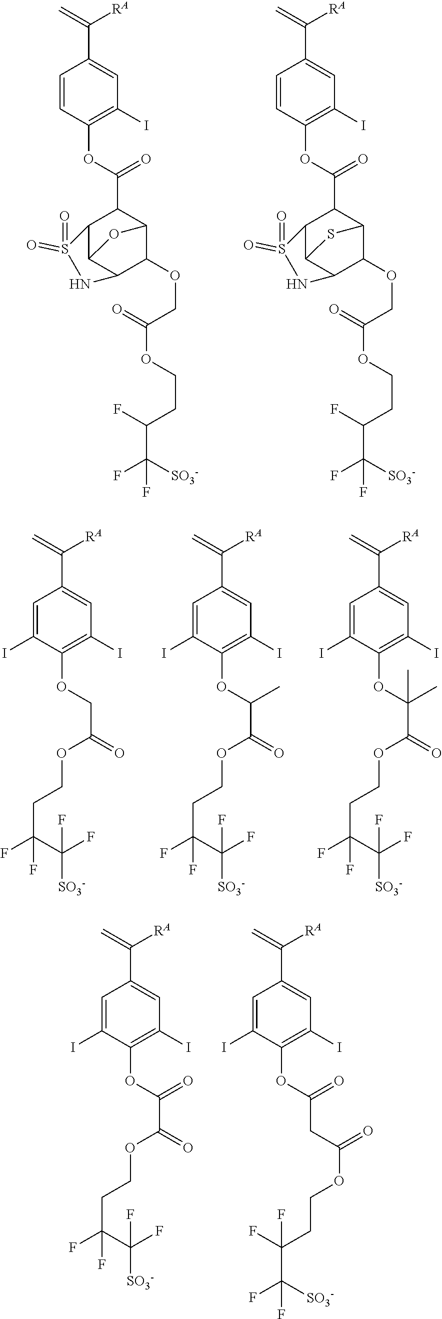

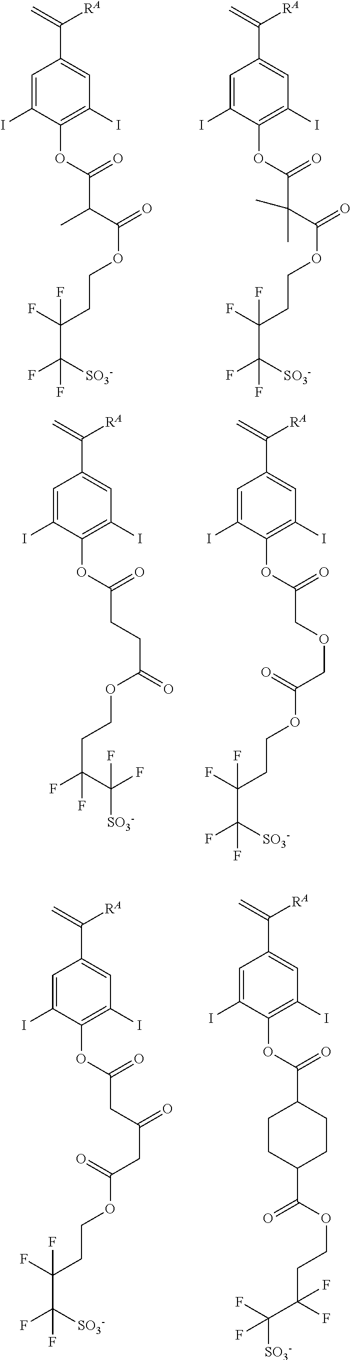

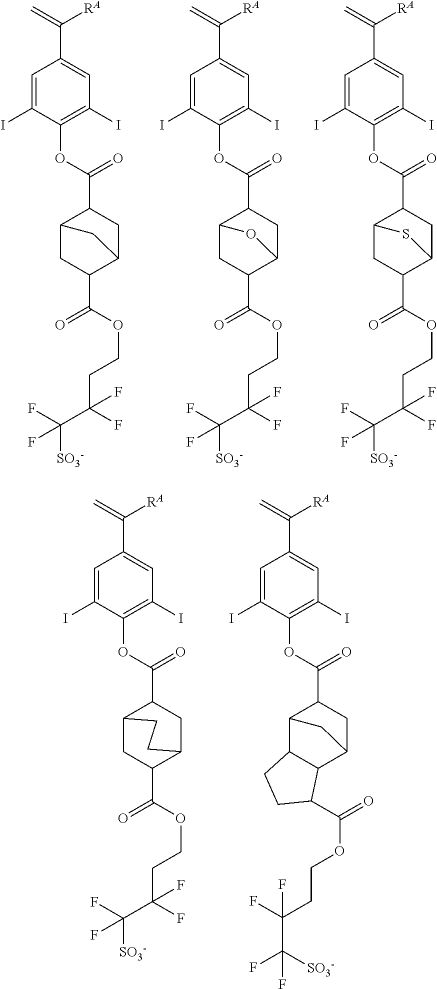

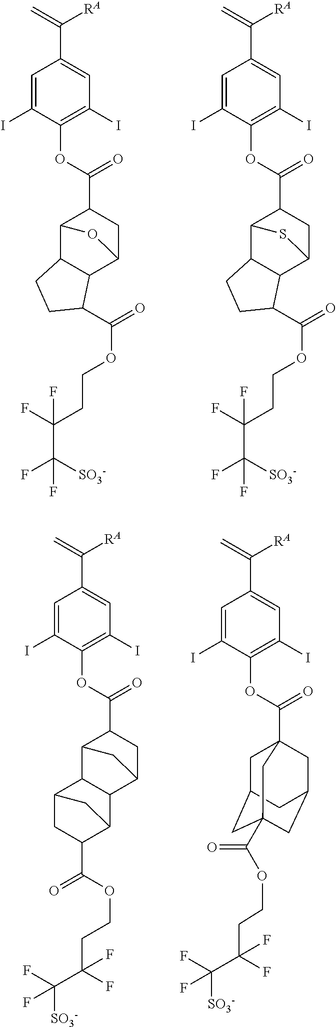

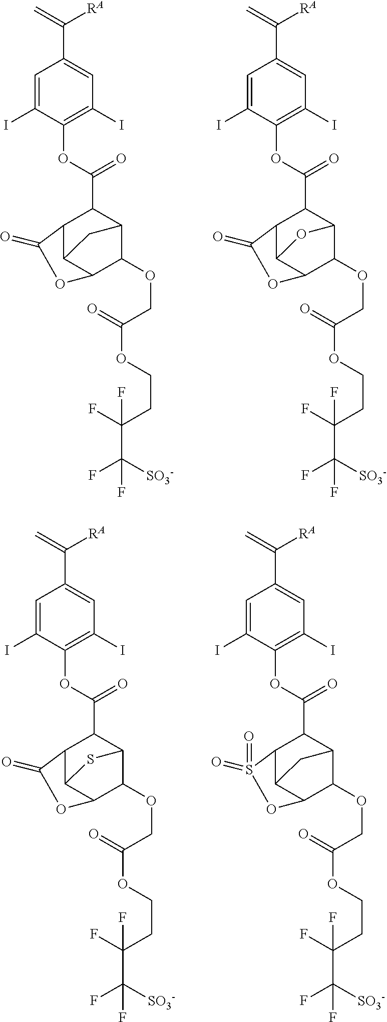

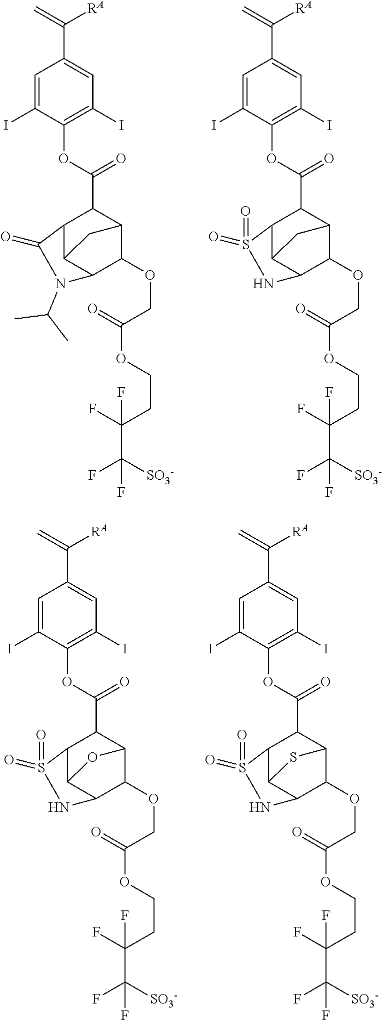

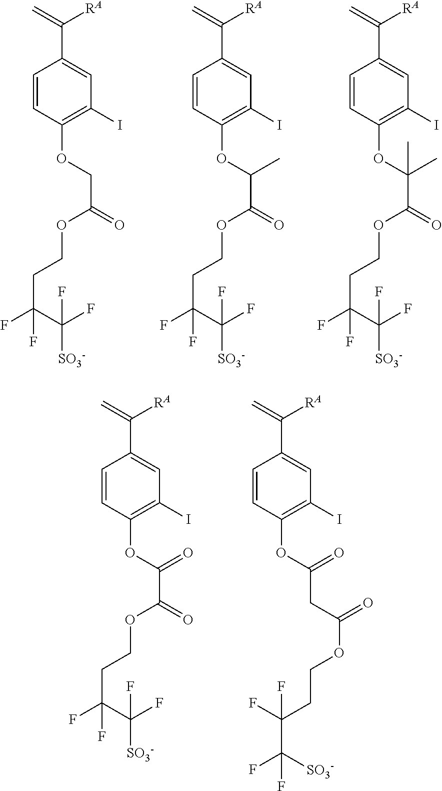

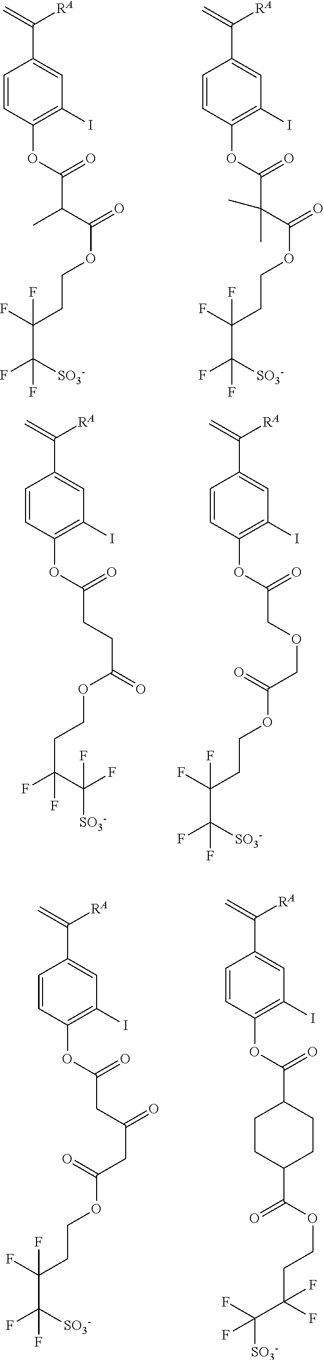

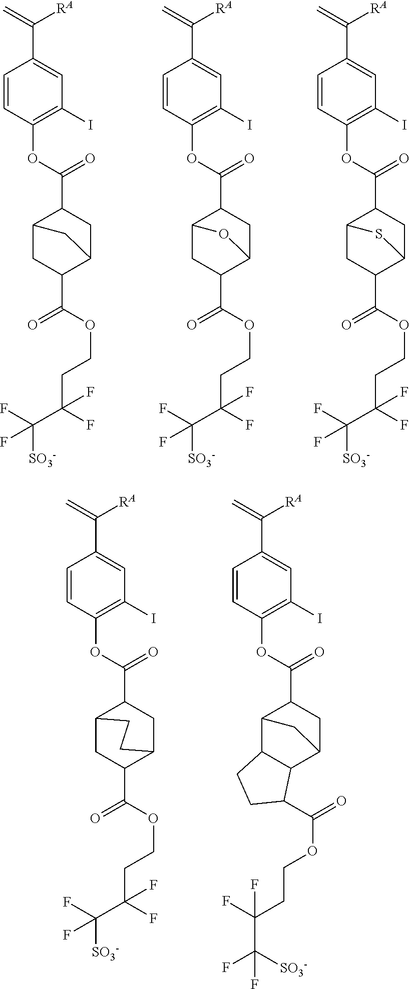

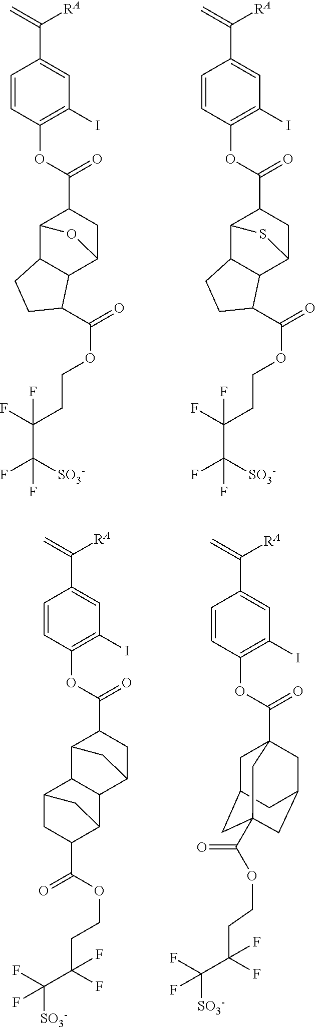

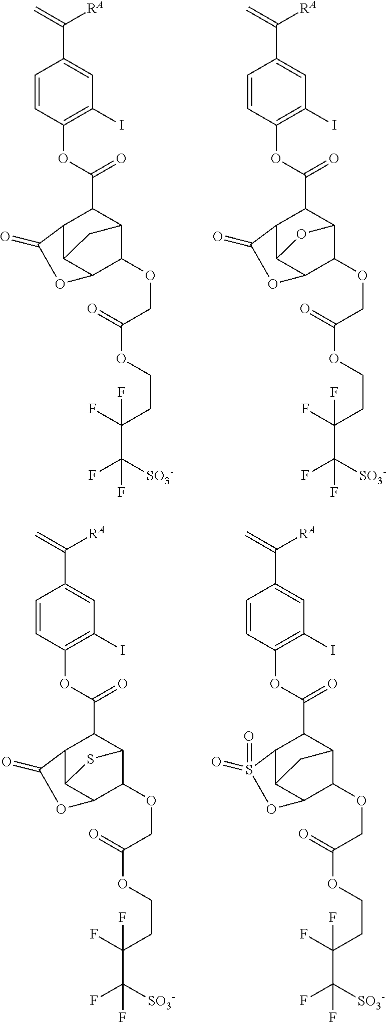

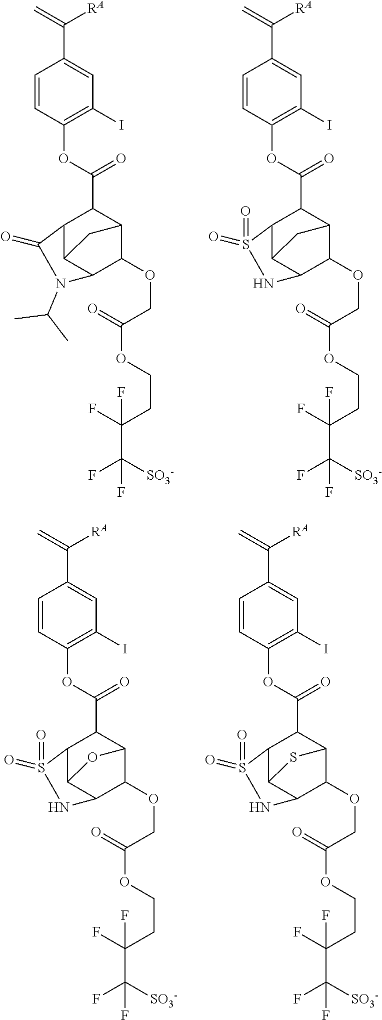

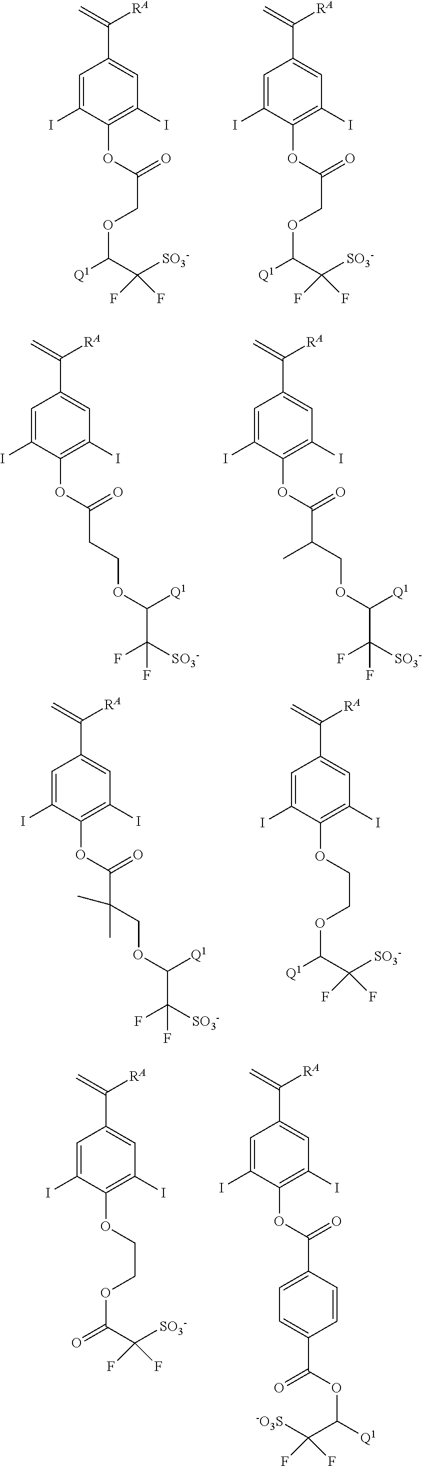

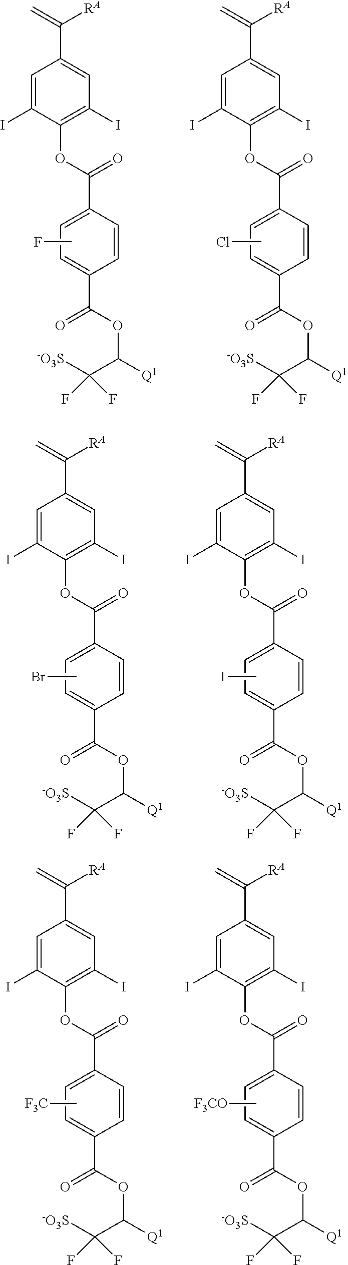

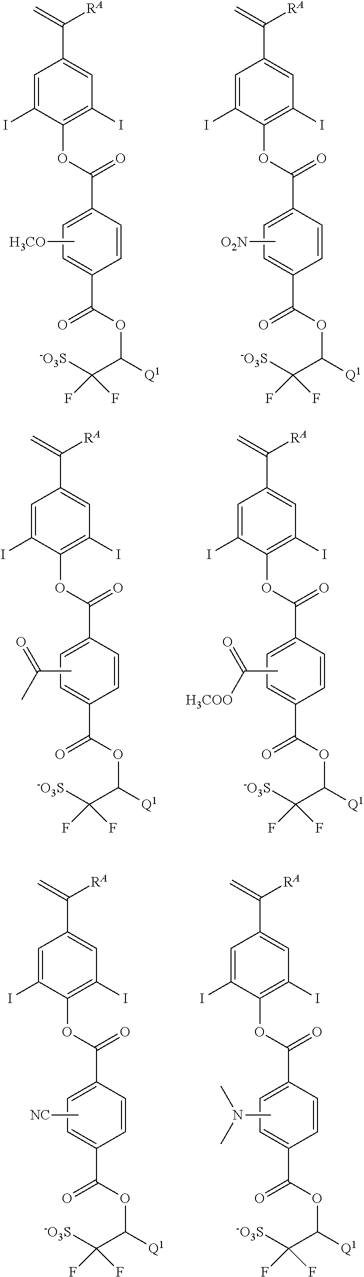

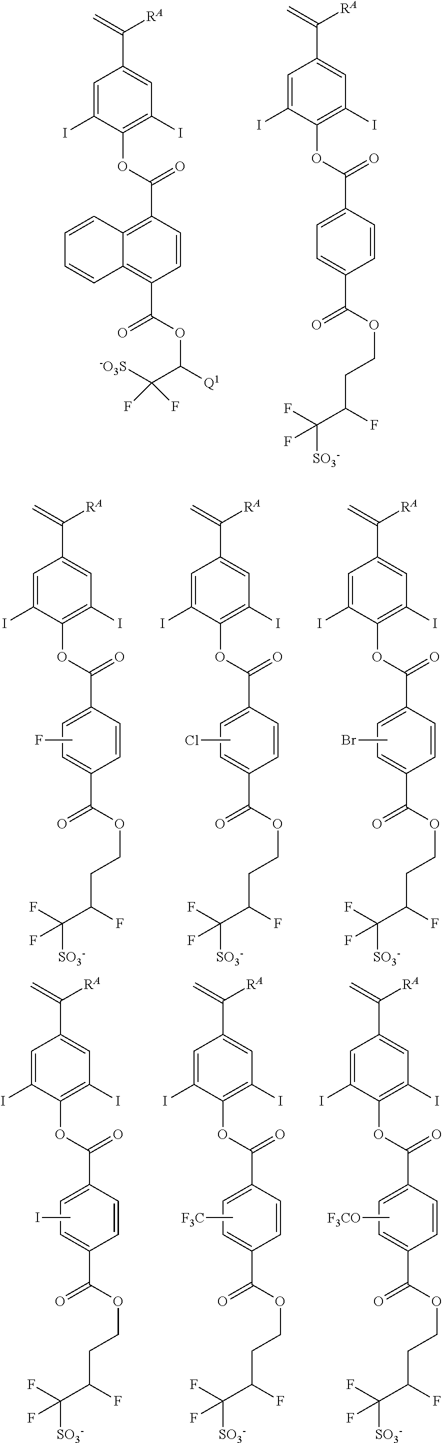

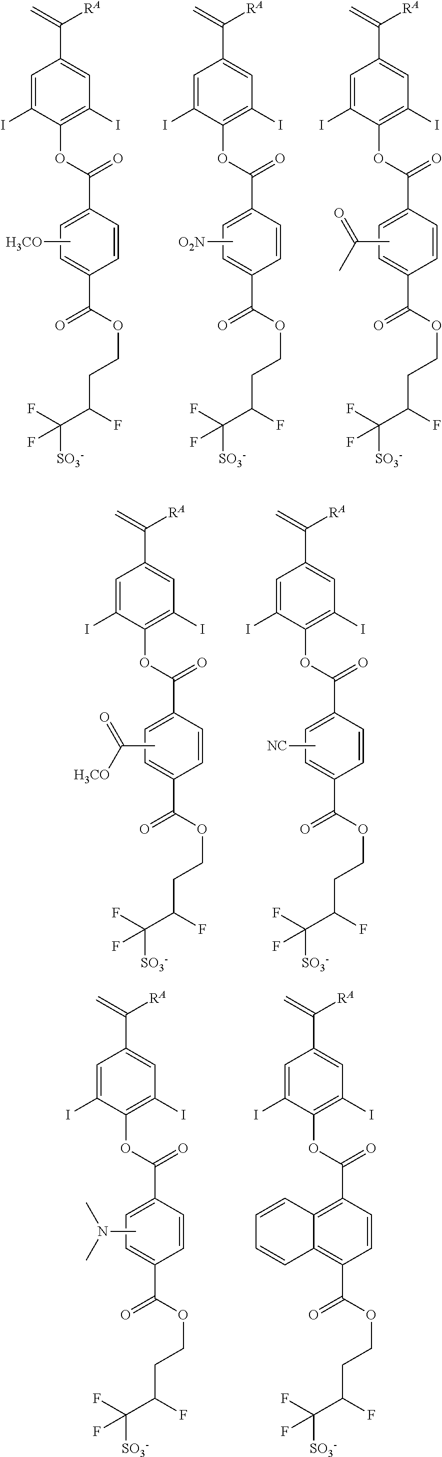

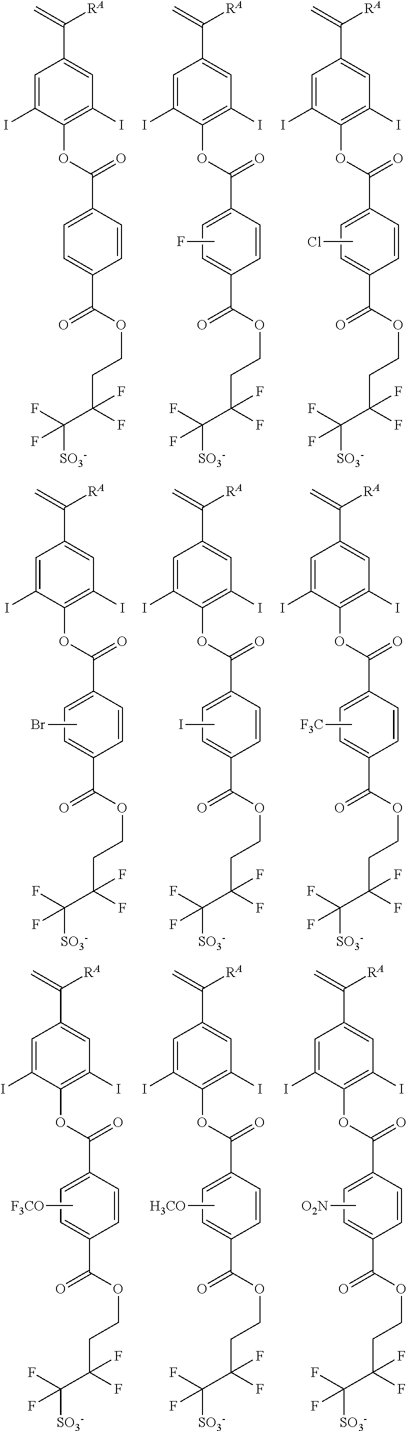

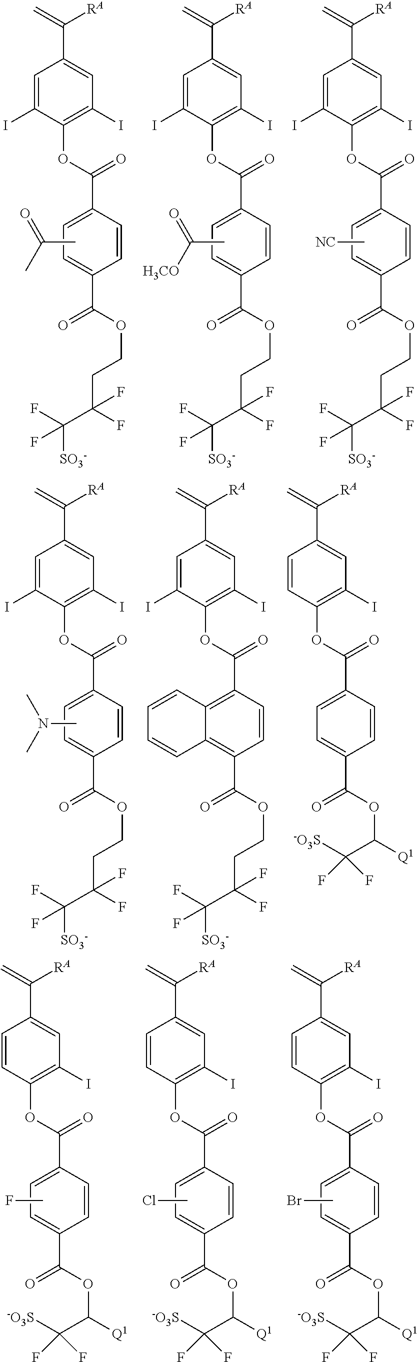

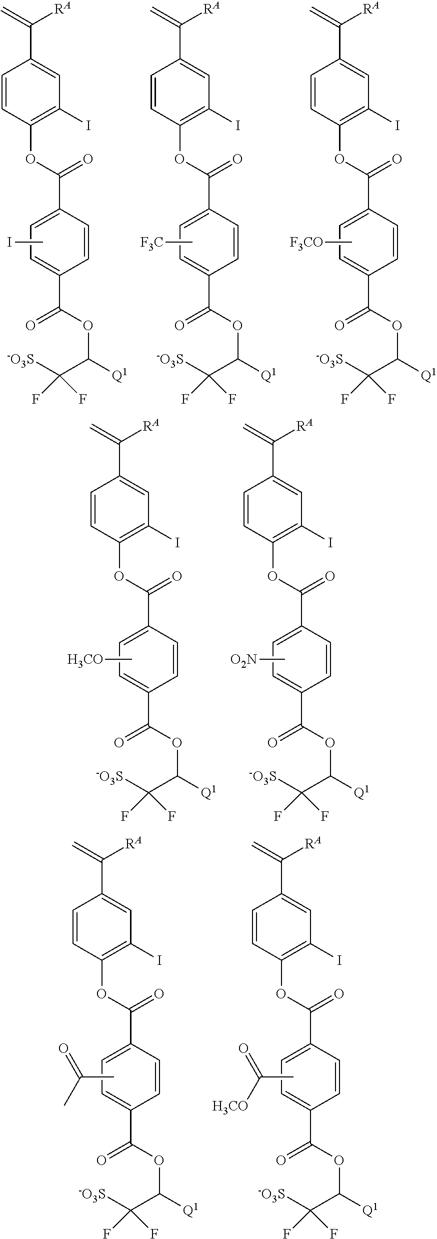

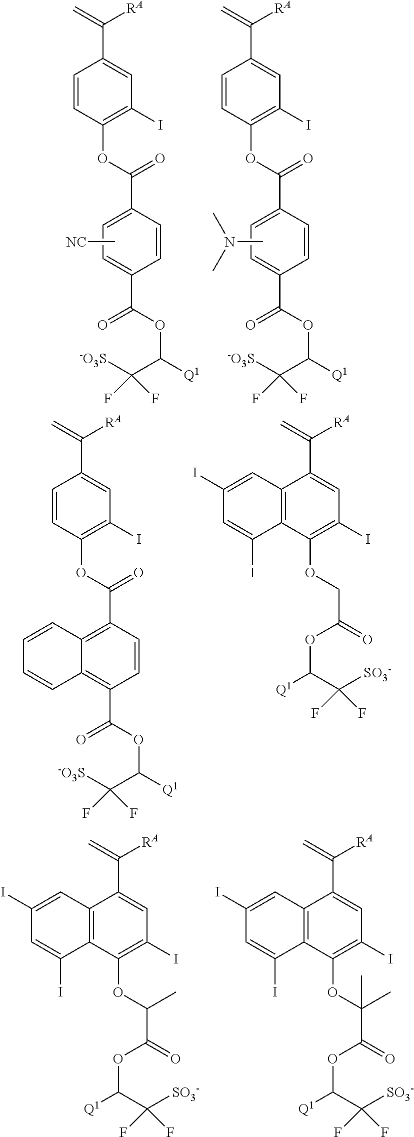

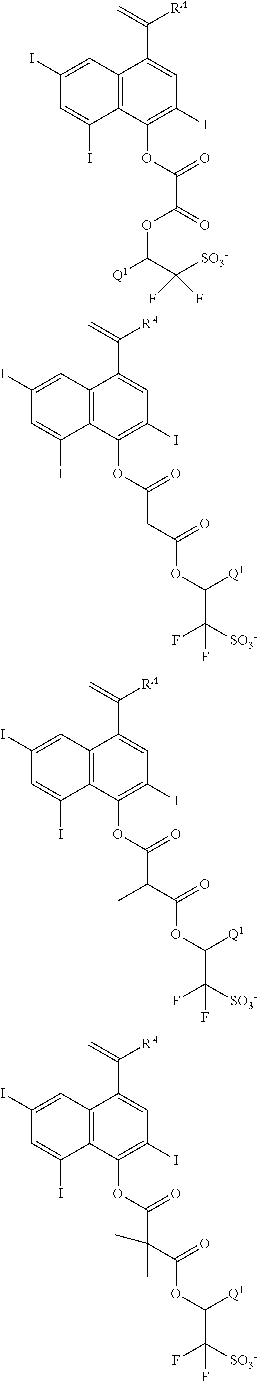

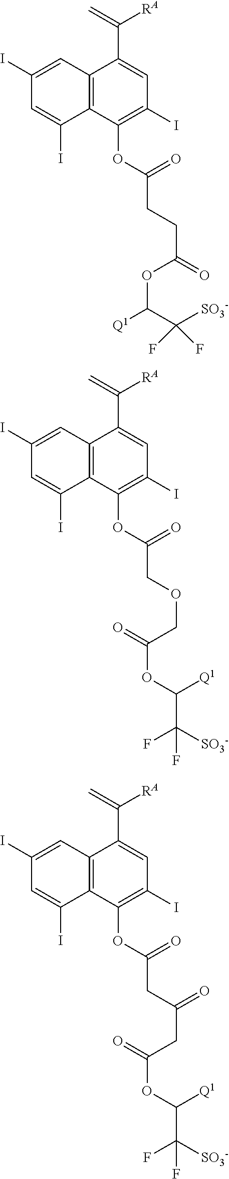

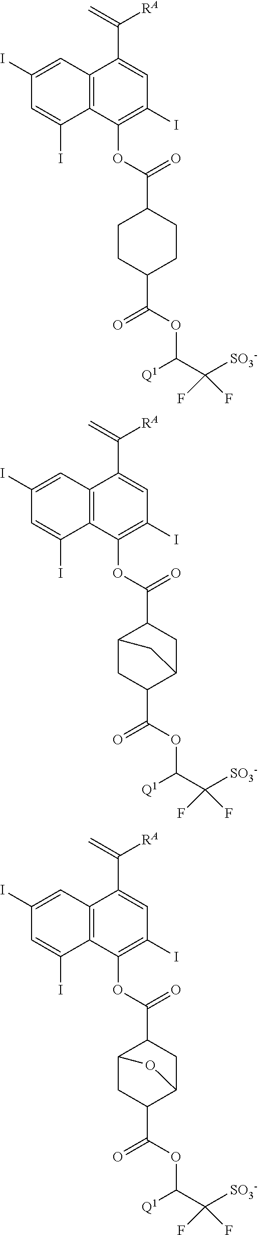

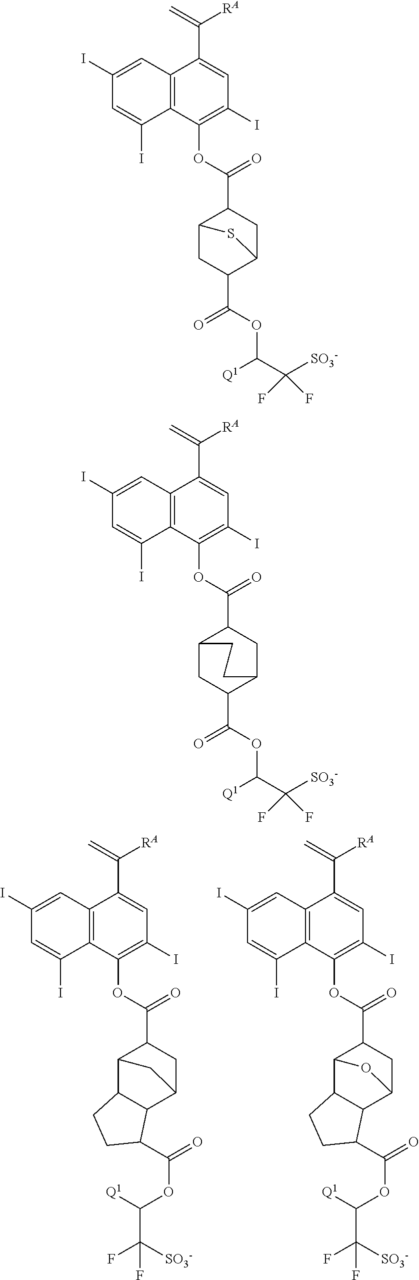

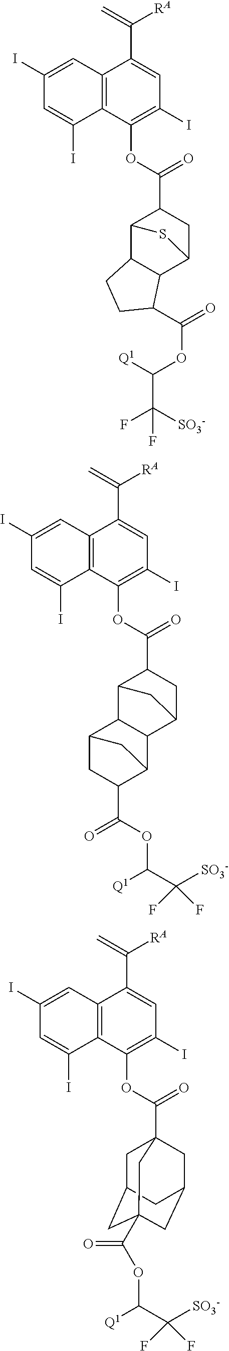

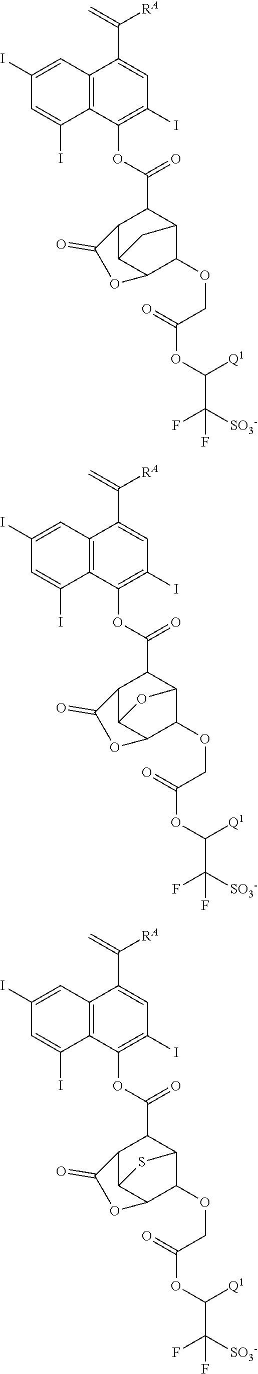

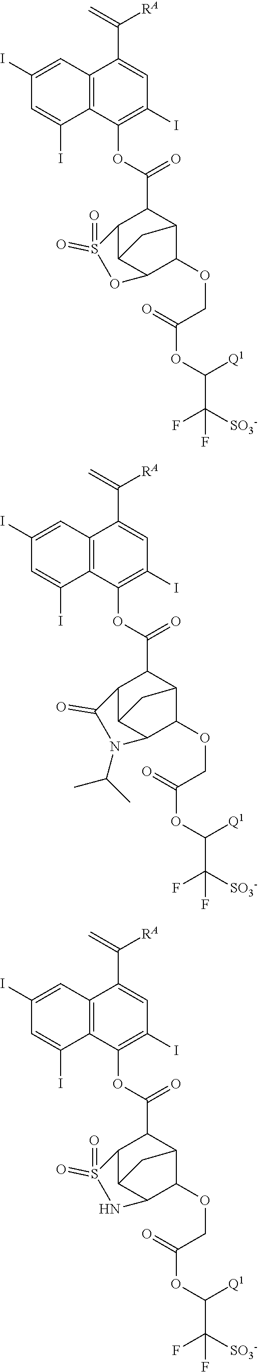

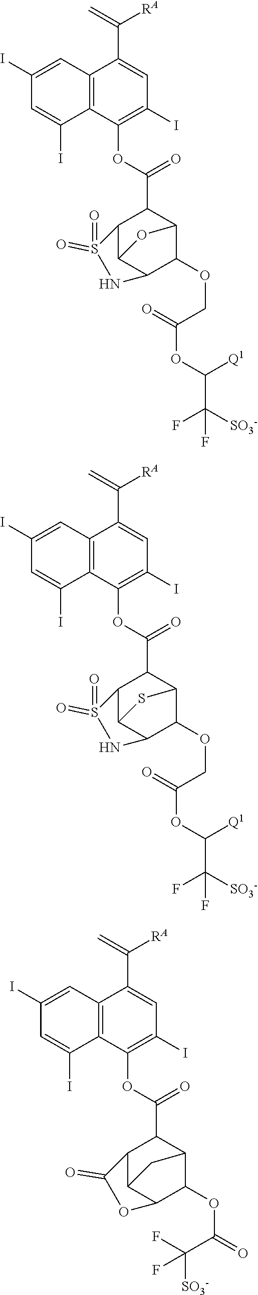

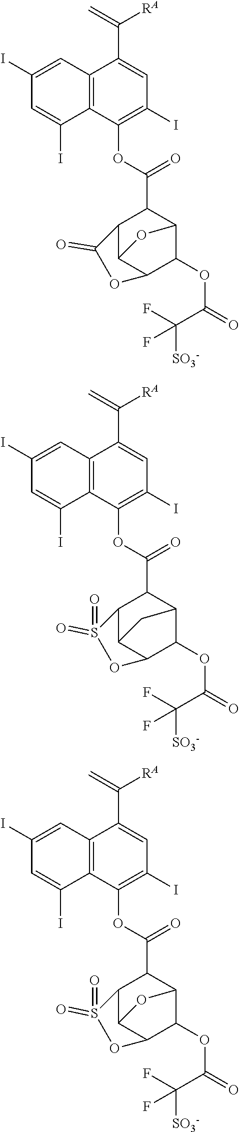

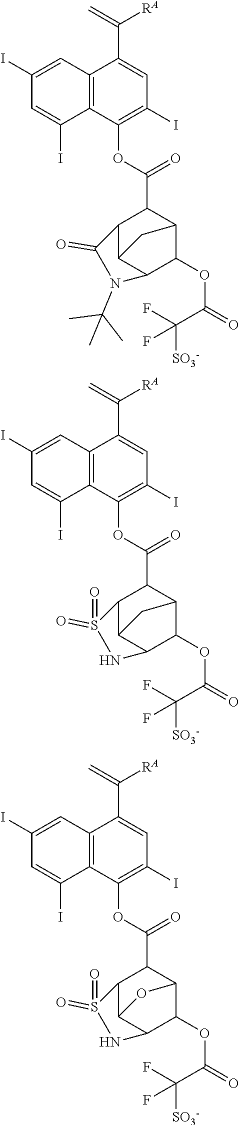

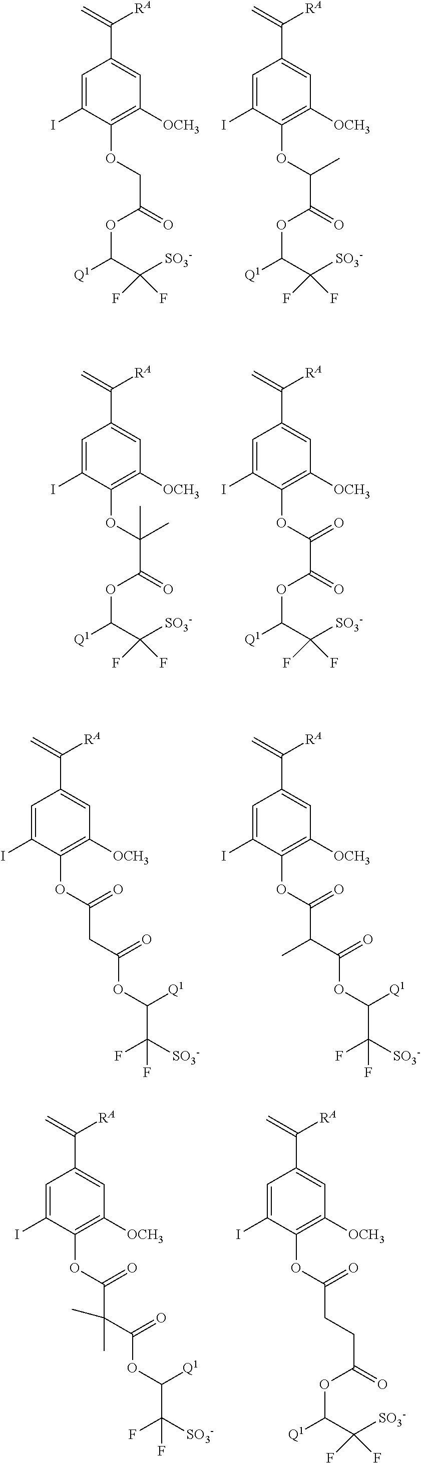

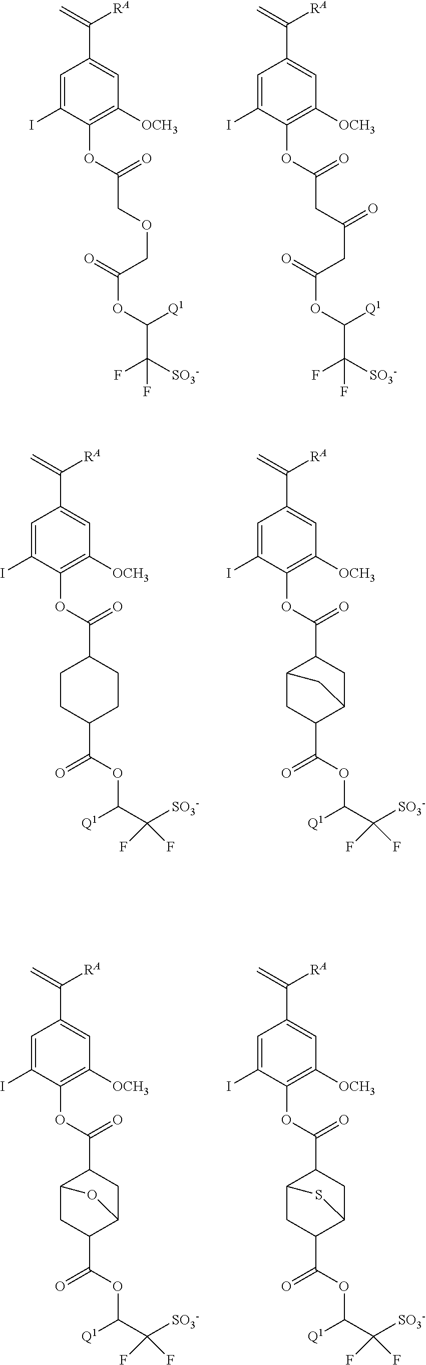

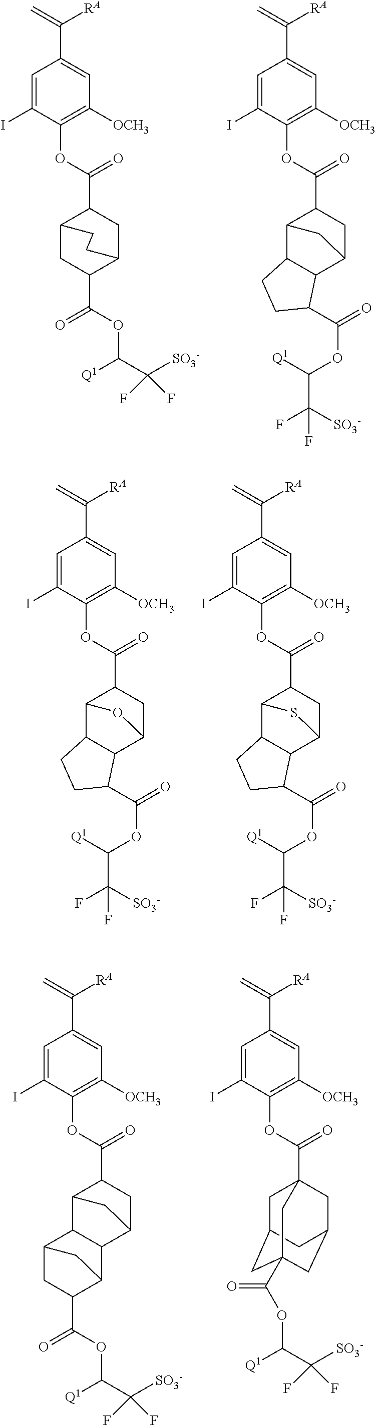

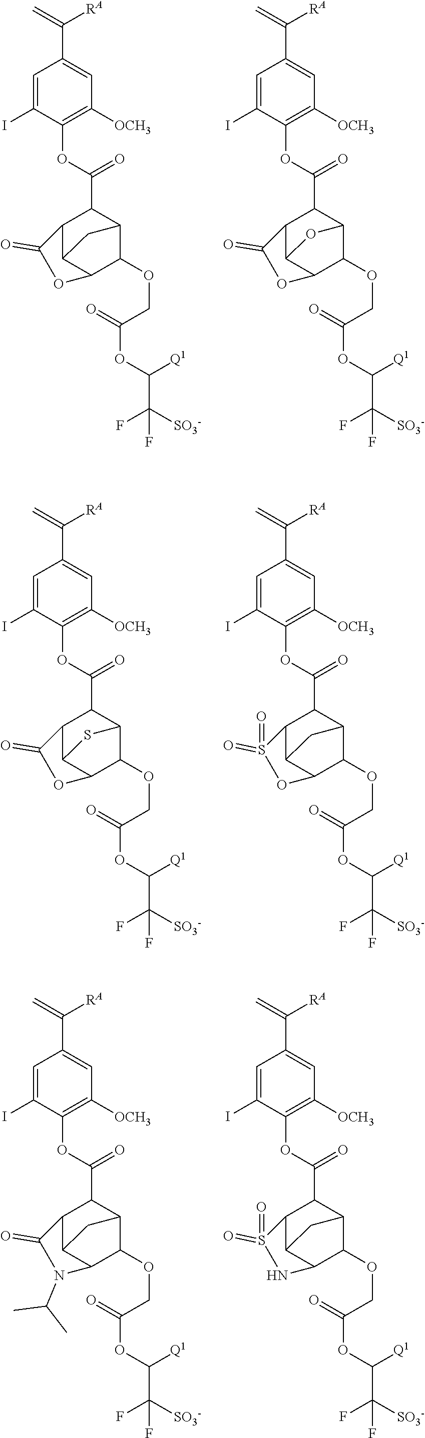

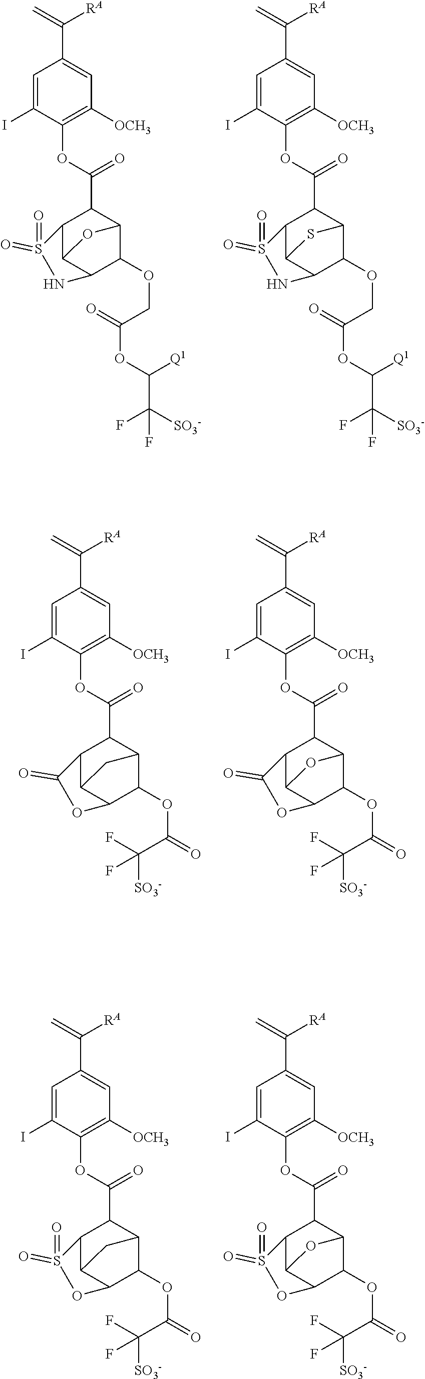

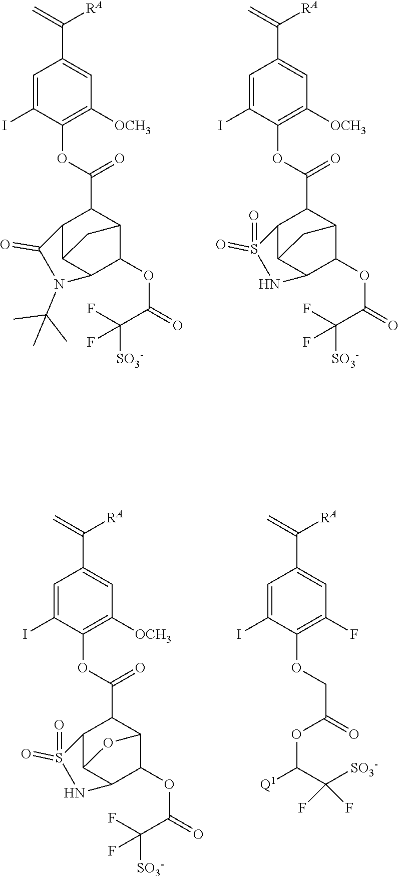

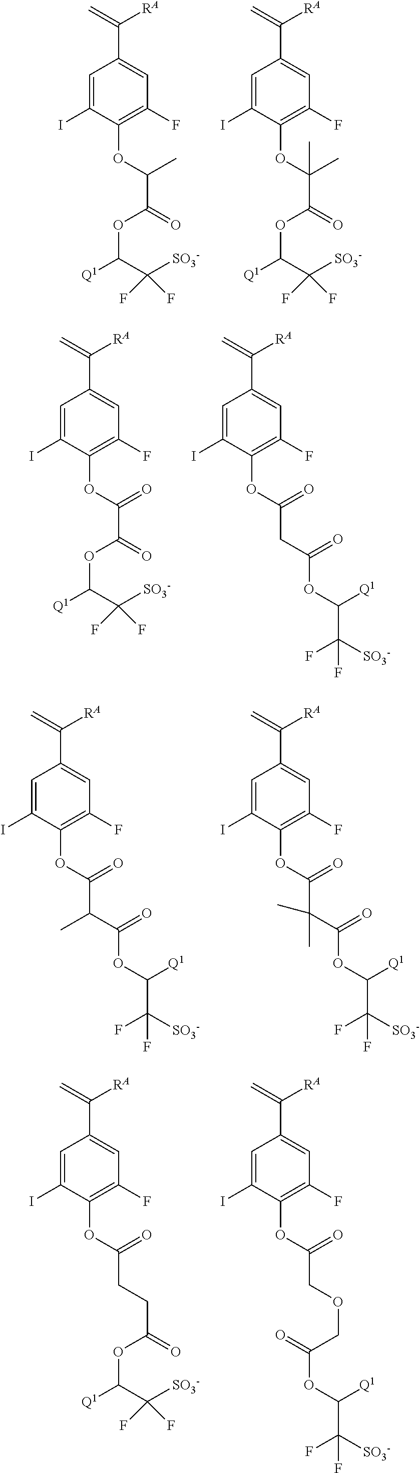

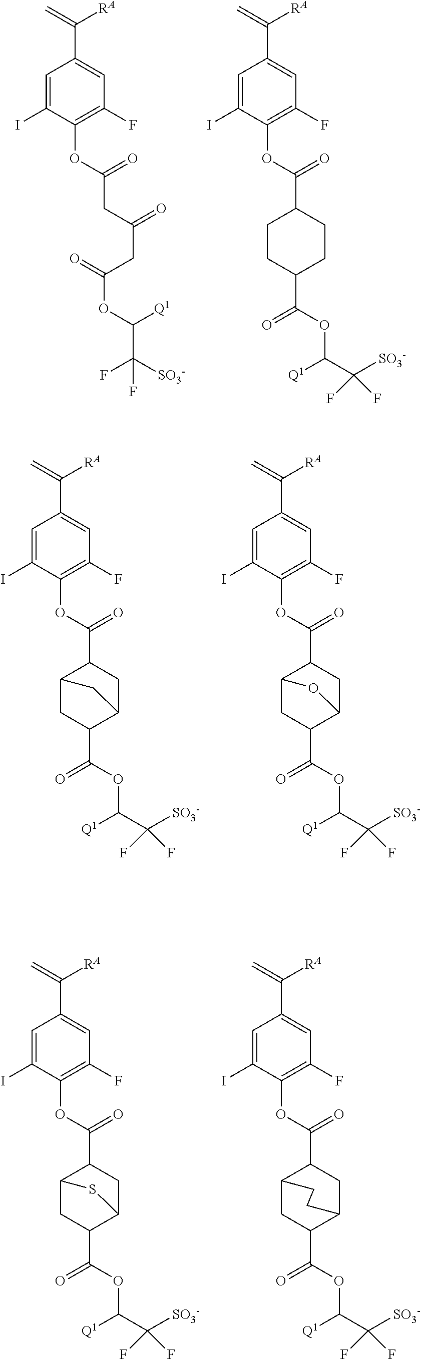

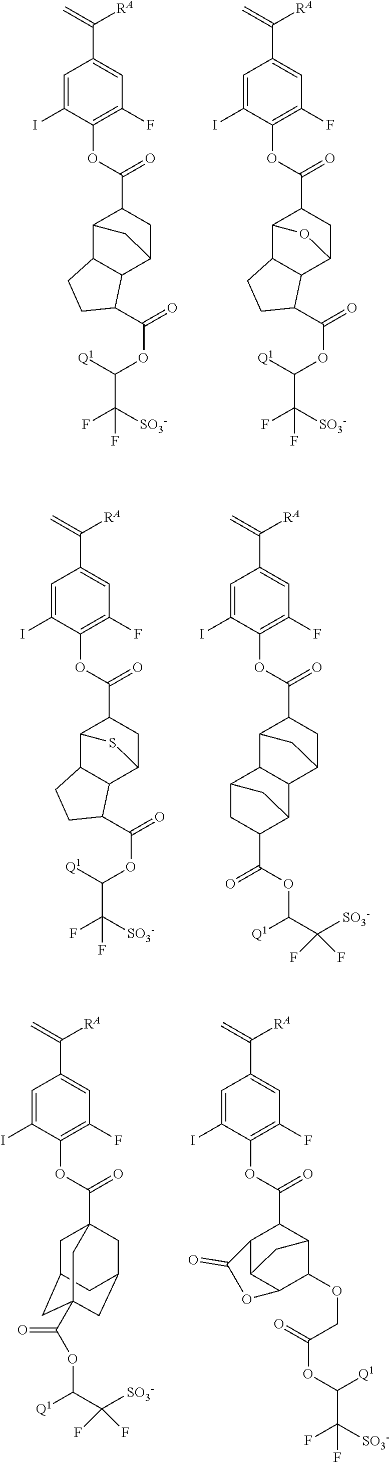

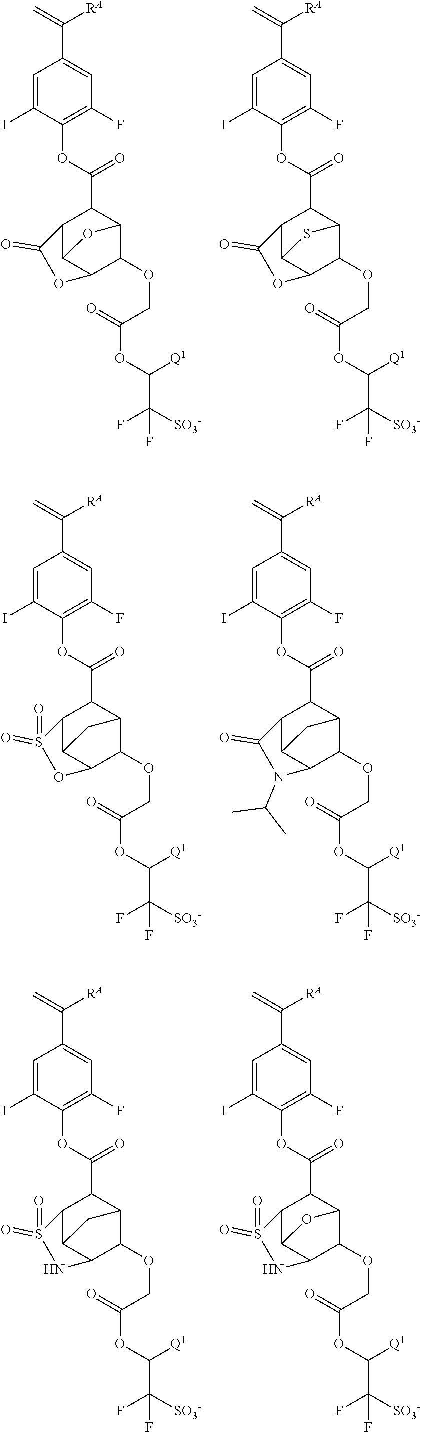

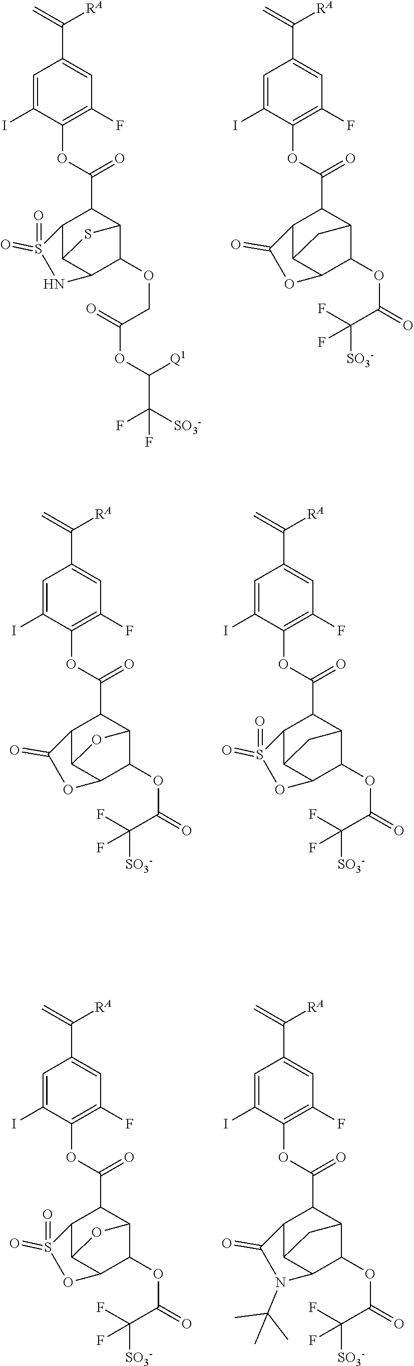

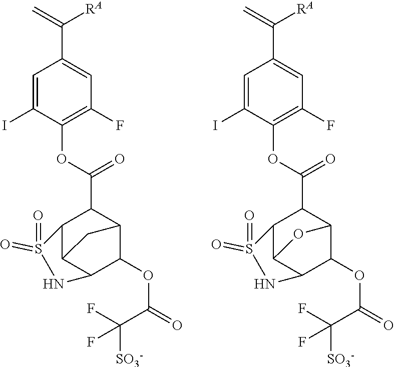

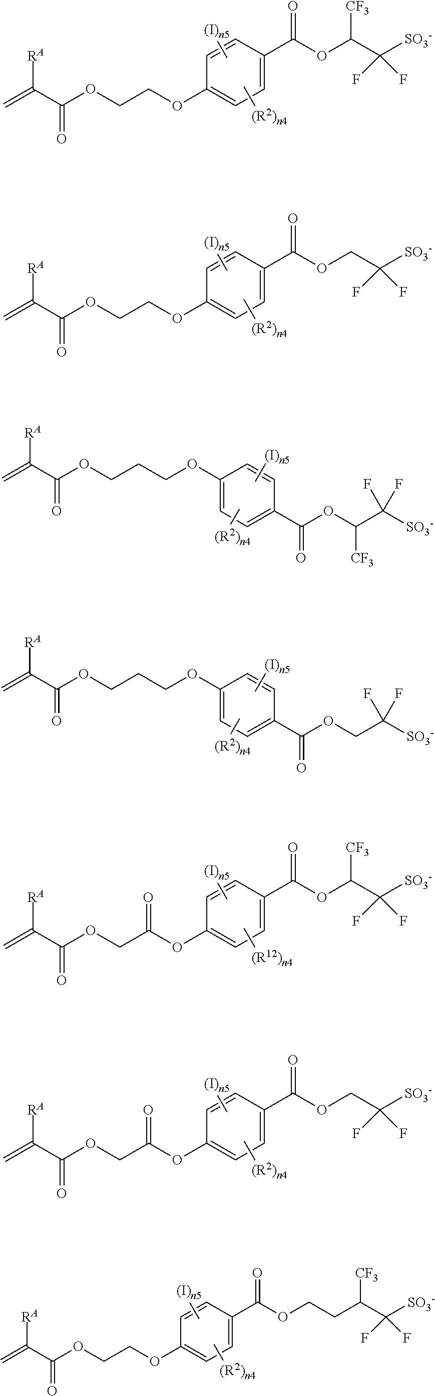

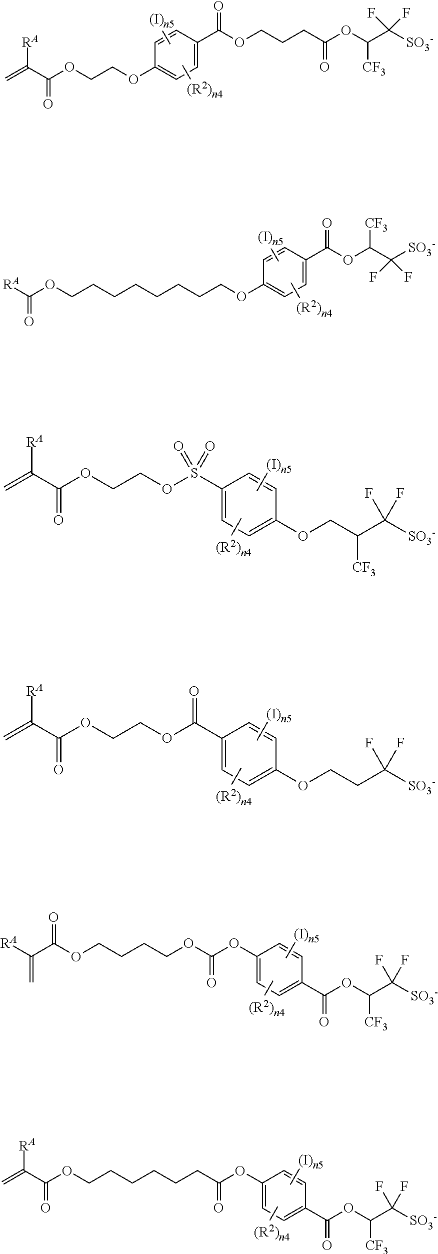

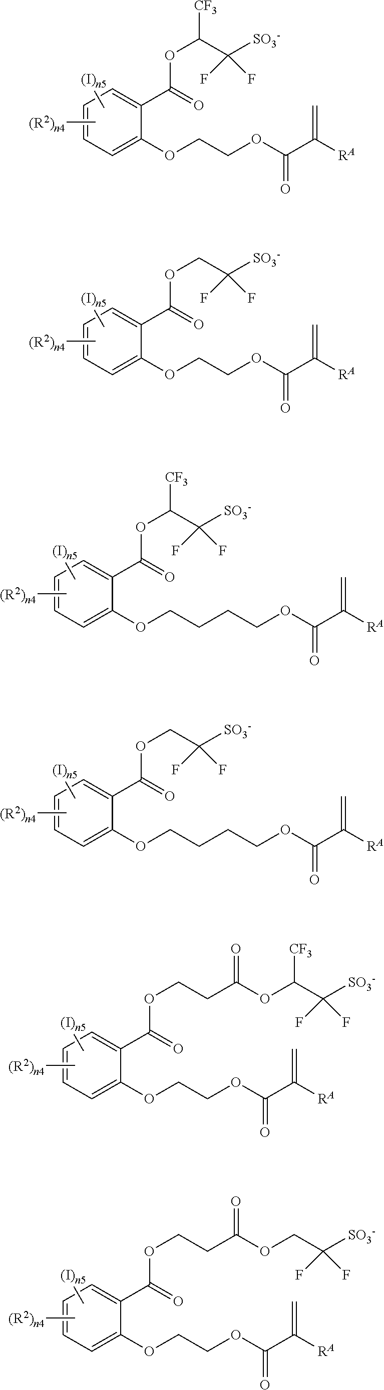

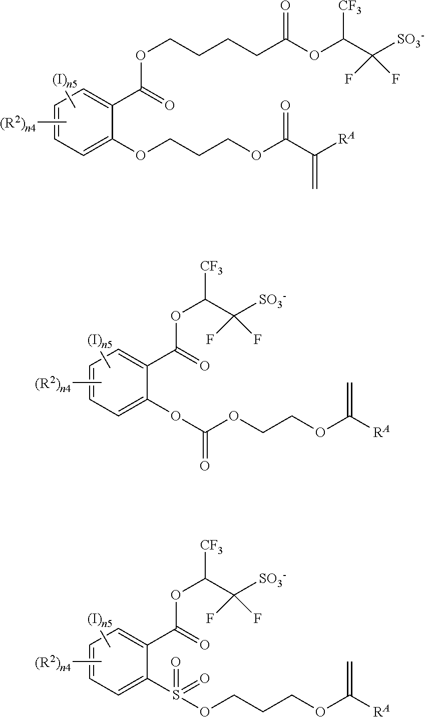

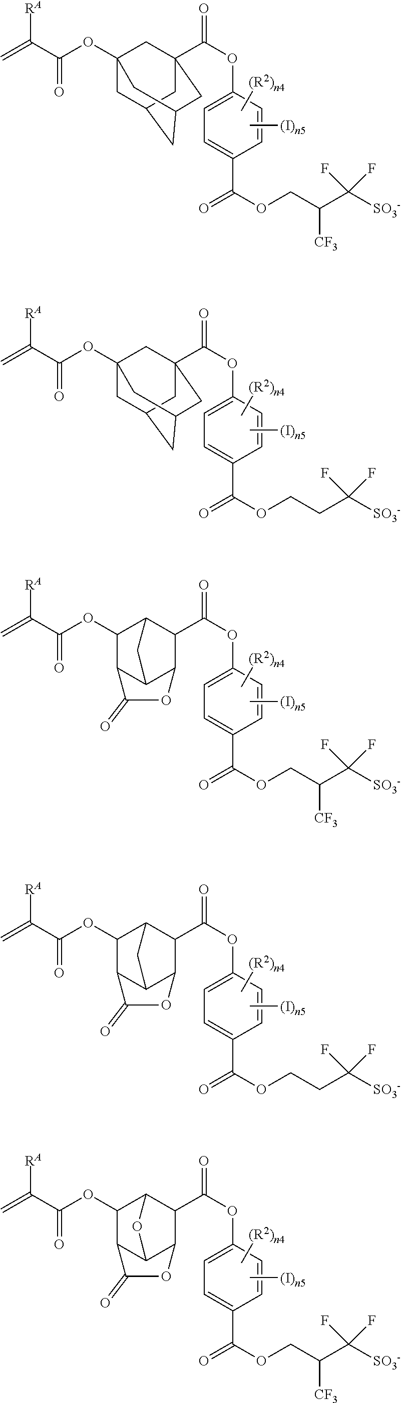

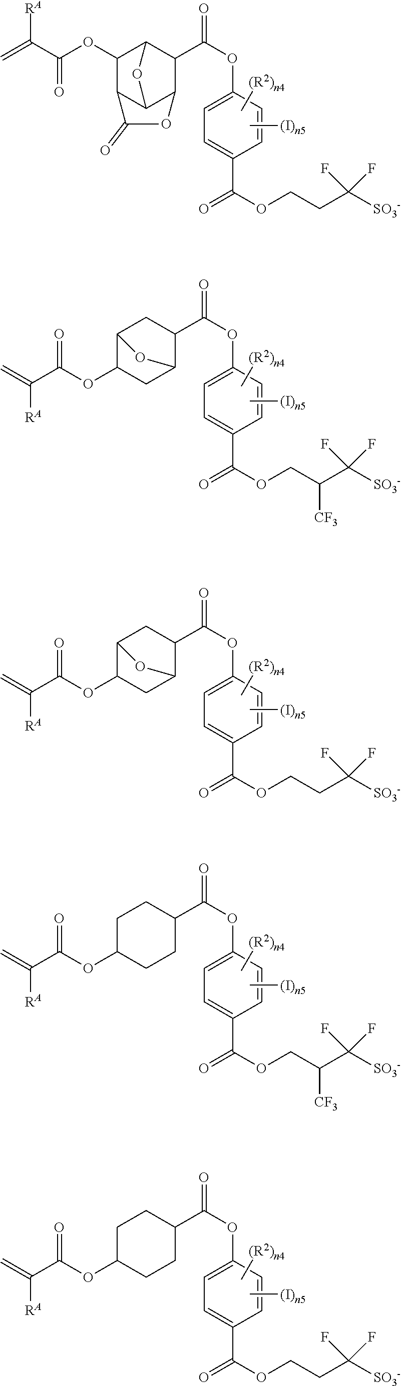

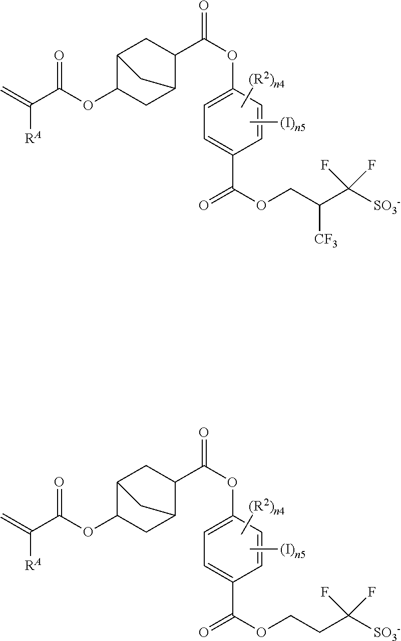

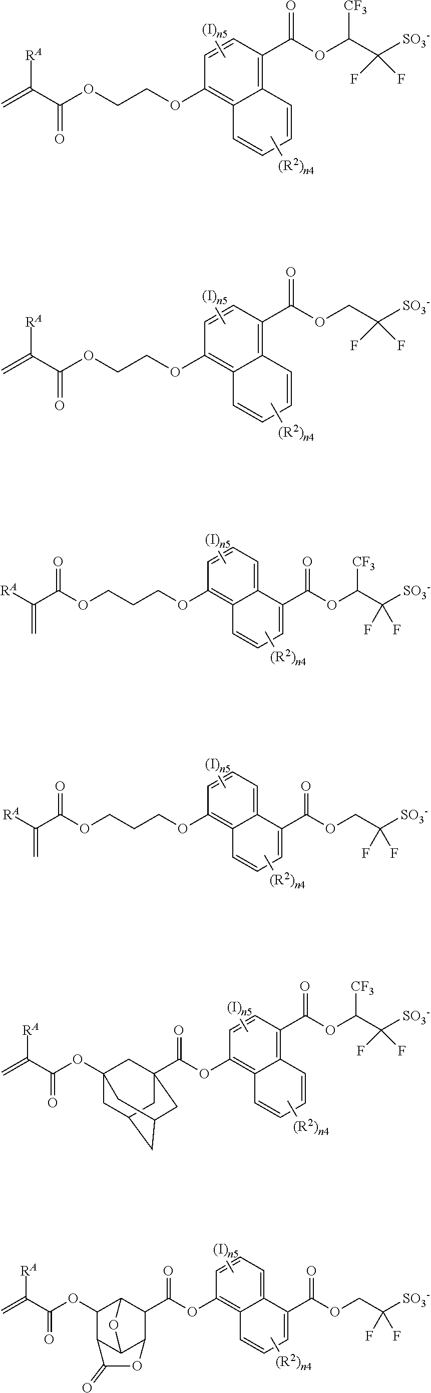

Other exemplary anions include those anions described in U.S. Pat. No. 11,022,883 (JP 6973274, paragraphs [0023-0029]).

The onium salt monomer contains a cation having the formula (a2).

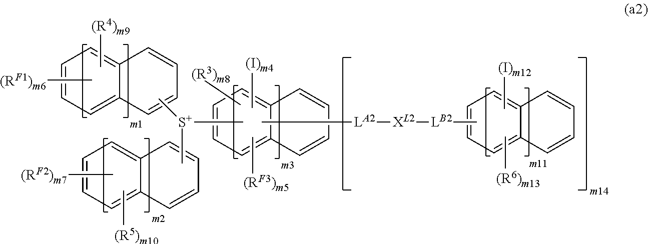

In formula (a2), m1 is 0 or 1. The relevant structure is a benzene ring in case of m1=0 and a naphthalene ring in case of m1=1. From the aspect of solvent solubility, the benzene ring corresponding to m1=0 is preferred. The subscript m2 is 0 or 1. The relevant structure is a benzene ring in case of m2=0 and a naphthalene ring in case of m2=1. From the aspect of solvent solubility, the benzene ring corresponding to m2=0 is preferred. The subscript m3 is 0 or 1. The relevant structure is a benzene ring in case of m3=0 and a naphthalene ring in case of m3=1. From the aspect of solvent solubility, the benzene ring corresponding to m3=0 is preferred.

In formula (a2), m4 is an integer of 0 to 4. Since a cation structure containing more iodine atoms is more absorptive to EUV, but loses solvent solubility so that it may precipitate in a resist composition, it is preferred that m4 be 0, 1, 2 or 3, more preferably 0, 1 or 2.

In formula (a2), m5 is an integer of 0 to 4. It is preferred from the aspect of reactant availability that m5 be 0, 1, 2 or 3, more preferably 0, 1 or 2. The subscript m6 is an integer of 0 to 6. It is preferred from the aspect of reactant availability that m6 be 0, 1, 2 or 3, more preferably 0, 1 or 2. The subscript m7 is an integer of 0 to 6. It is preferred from the aspect of reactant availability that m7 be 0, 1, 2 or 3, more preferably 0, 1 or 2.

In formula (a2), m8 is an integer of 0 to 2. It is preferred from the aspect of reactant availability that m8 be 0 or 1. The subscript m9 is an integer of 0 to 2. It is preferred from the aspect of reactant availability that m9 be 0 or 1. The subscript m10 is an integer of 0 to 2. It is preferred from the aspect of reactant availability that m10 be 0 or 1.

In formula (a2), m11 is 0 or 1. The relevant structure is a benzene ring in case of m11=0 and a naphthalene ring in case of m11=1. From the aspect of solvent solubility, the benzene ring corresponding to m11=0 is preferred.

In formula (a2), m12 is an integer of 0 to 4. Since a cation structure containing more iodine atoms is more absorptive to EUV, but loses solvent solubility so that it may precipitate in a resist composition, it is preferred that m12 be 0, 1, 2 or 3, more preferably 0, 1 or 2.

In formula (a2), m13 is an integer of 0 to 2. It is preferred from the aspect of reactant availability that m13 be 0 or 1. The subscript m14 is an integer of 0 to 2. It is preferred from the aspect of synthesis that m14 be 0 or 1.

It is noted that m6+m9 is from 0 to 4 when m1=0, m6+m9 is from 0 to 6 when m1=1; m7+m10 is from 0 to 4 when m2=0, m7+m10 is from 0 to 6 when m2=1; m4+m5+m8+m14 is from 1 to 4 when m3=0, m4+m5+m8+m14 is from 1 to 6 when m3=1; m12+m13 is from 0 to 4 when m11=0, m12+m13 is from 0 to 6 when m11=1; and m4+m12 is 1 or greater.

In formula (a2), R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group. Of these, trifluoromethyl, trifluoromethoxy, and trifluorothiomethoxy are preferred. A plurality of R^{F1} may be identical or different when m6 is 2 or more, a plurality of R^{F2} may be identical or different when m7 is 2 or more, and a plurality of R^{F3} may be identical or different when m5 is 2 or more.

In formula (a2), R³ to R⁶ are each independently halogen exclusive of iodine and fluorine, nitro, cyano, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom. The hydrocarbyl group and hydrocarbyl moiety in the hydrocarbyloxy and hydrocarbylthio groups may be saturated or unsaturated and straight, branched or cyclic. Examples thereof are as exemplified above for the hydrocarbyl group R¹. In the hydrocarbyl group and hydrocarbyl moiety in the hydrocarbyloxy and hydrocarbylthio groups, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

When m8=2, two R³ may be identical or different and two R³ may bond together to form a ring with the carbon atoms to which they are attached. When m9=2, two R⁴ may be identical or different and two R⁴ may bond together to form a ring with the carbon atoms to which they are attached. When m10=2, two R⁵ may be identical or different and two R⁵ may bond together to form a ring with the carbon atoms to which they are attached. When m13=2, two R⁶ may be identical or different and two R⁶ may bond together to form a ring with the carbon atoms to which they are attached. Examples of the ring thus formed include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

The aromatic rings directly bonded to S⁺ in the sulfonium cation having formula (a2) may bond together to form a ring with S⁺. Exemplary structures of the ring are shown below.

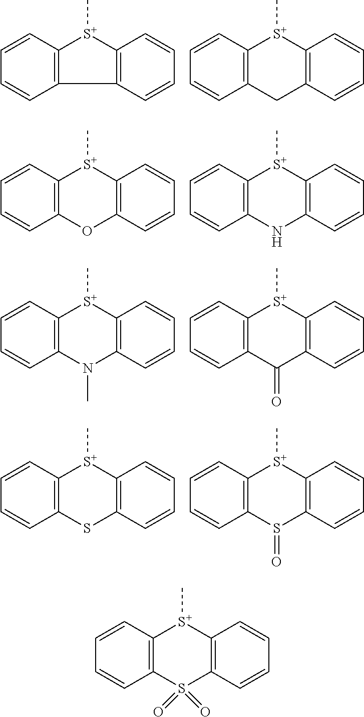

In formula (a2), L^{A2} and L^{B2} are each independently a single bond, ether bond, ester bond, amide bond, sulfonate ester bond, sulfonamide bond, carbonate bond or carbamate bond. L^{A2} is preferably a single bond, ether bond, ester bond or sulfonate ester bond, more preferably an ester bond or sulfonate ester bond. L^{B2} is preferably a single bond, ether bond or ester bond, more preferably a single bond.

In formula (a2), X^{L2} is a single bond or a C₁-C₄₀ hydrocarbylene group which may contain a heteroatom. Examples of the C₁-C₄₀ hydrocarbylene group are as exemplified above for the hydrocarbylene group X^{L1}, but not limited thereto.

Preferably the sulfonium cation having formula (a2) has the formula (a2-1):

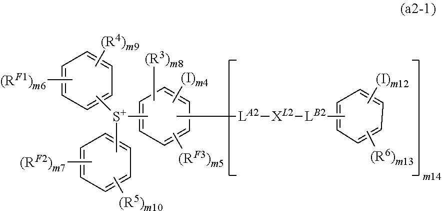

wherein m4 to m10, m12 to m14, R^{F1} to R^{F3}, R³ to R⁶, L^{A2}, L^{B2} and X^{L2} are as defined above.

More preferably, the sulfonium cation having formula (a2-1) has the formula (a2-2):

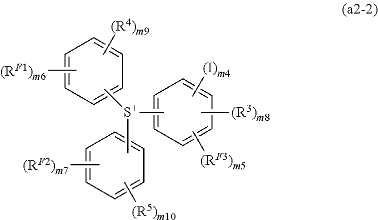

wherein m4 to m10, R^{F1} to R^{F3}, and R³ to R⁵ are as defined above.

Examples of the sulfonium cation having formula (a2) are shown below, but not limited thereto.

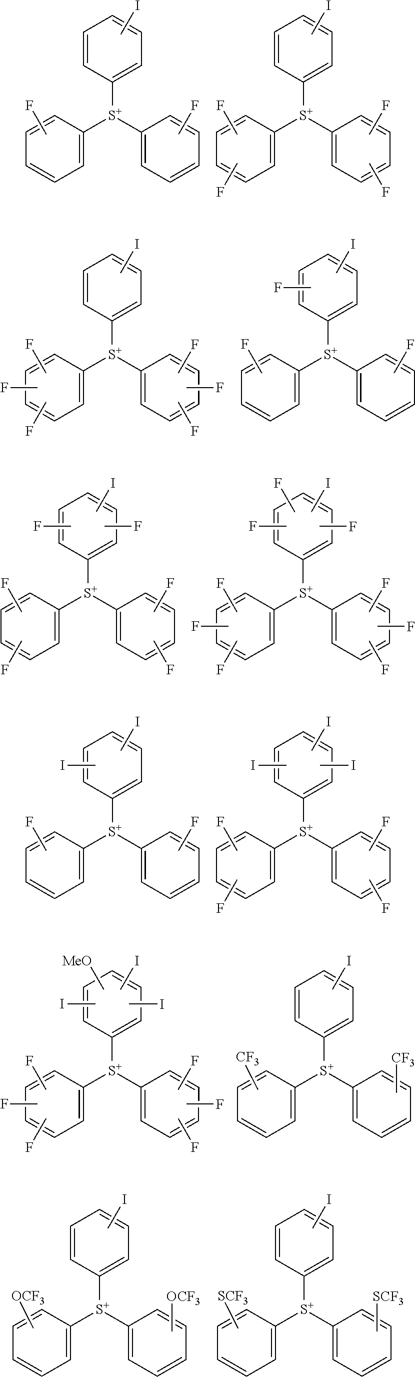

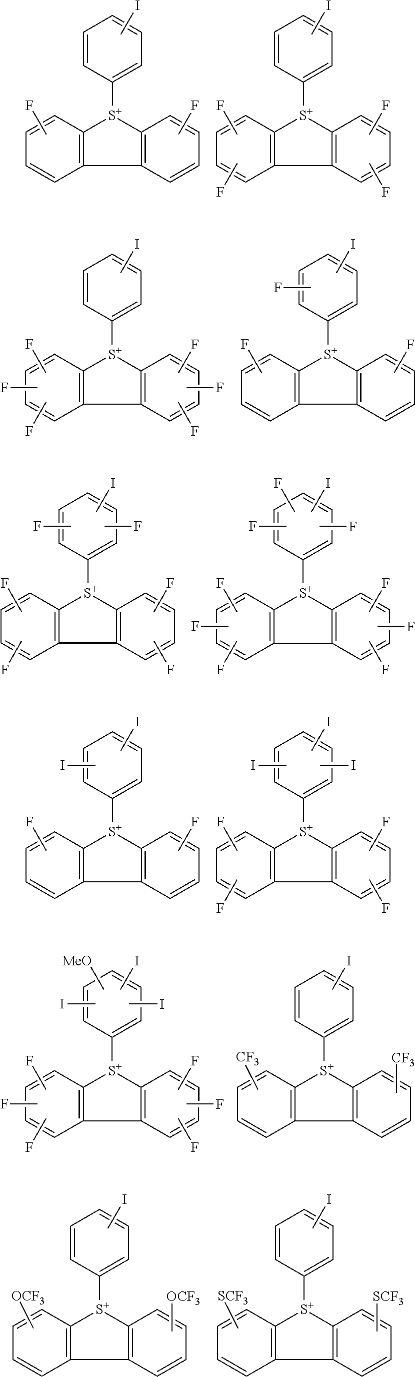

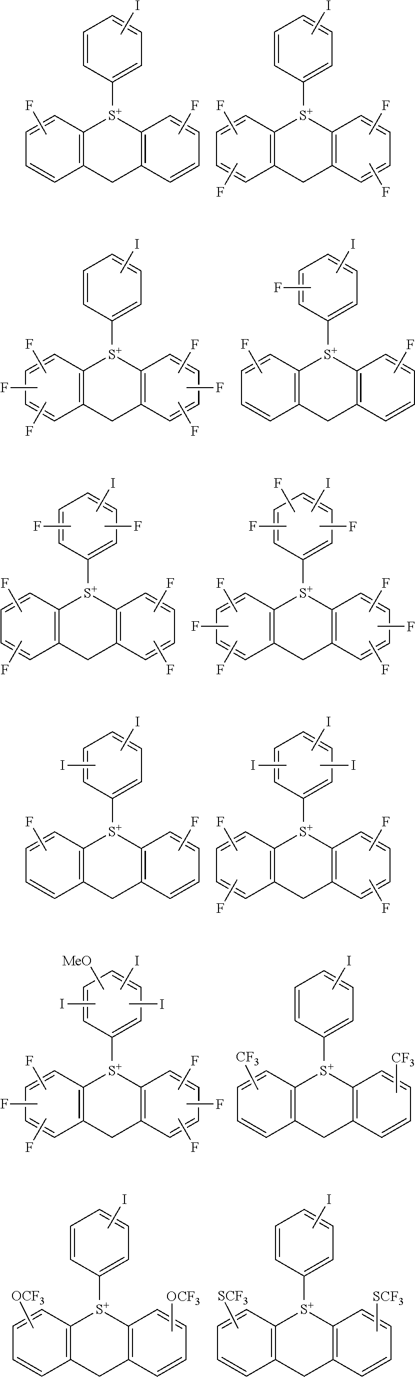

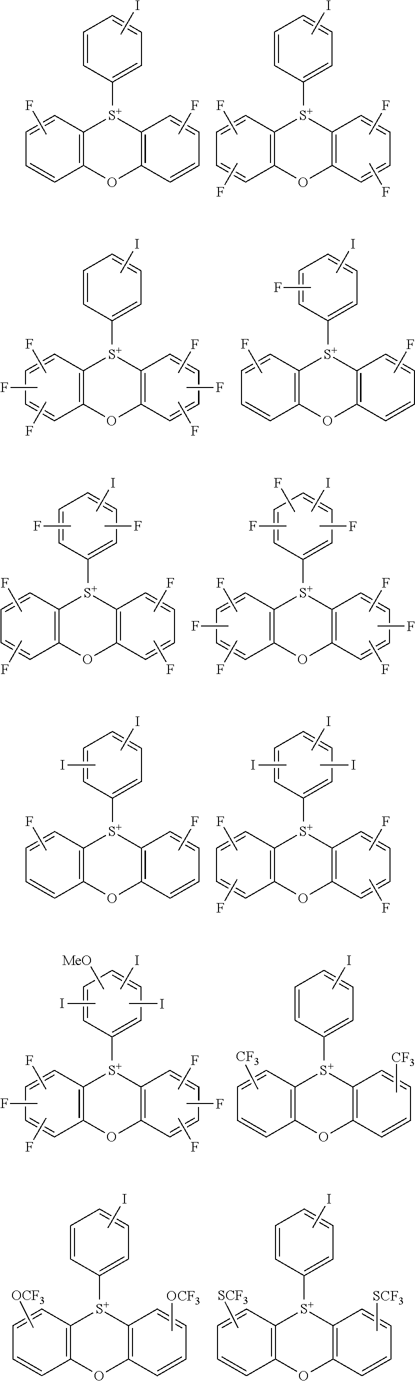

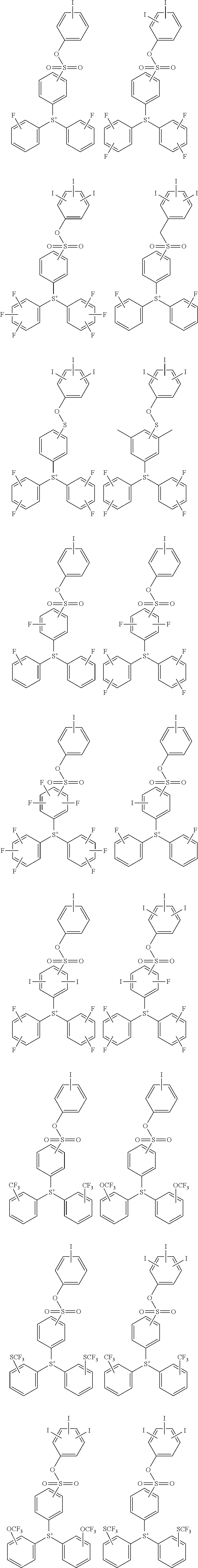

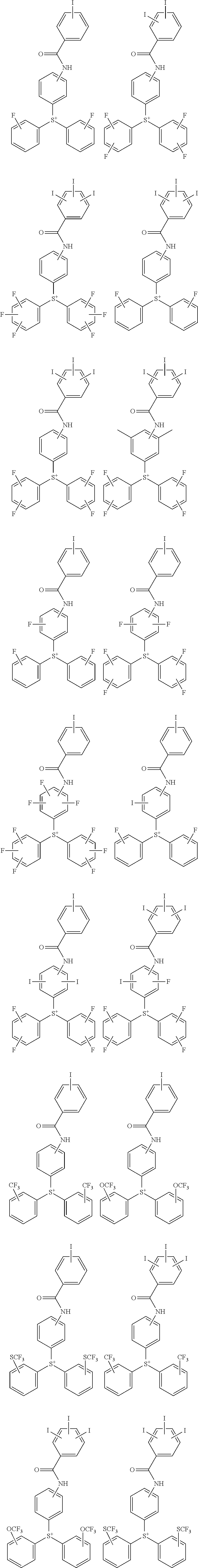

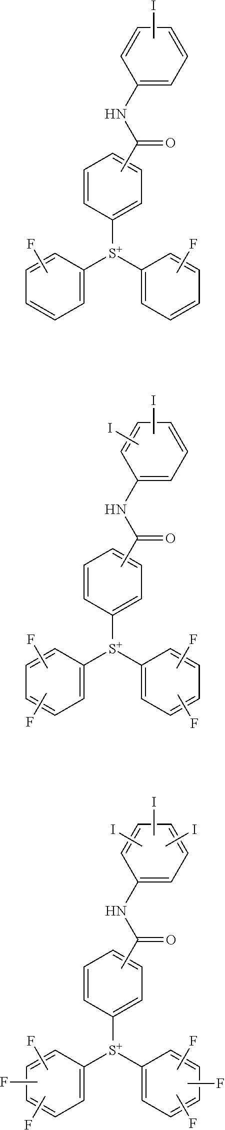

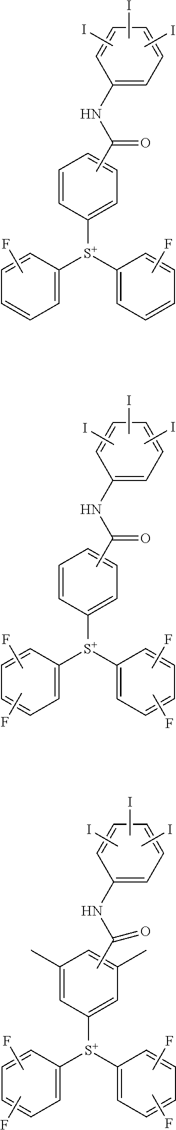

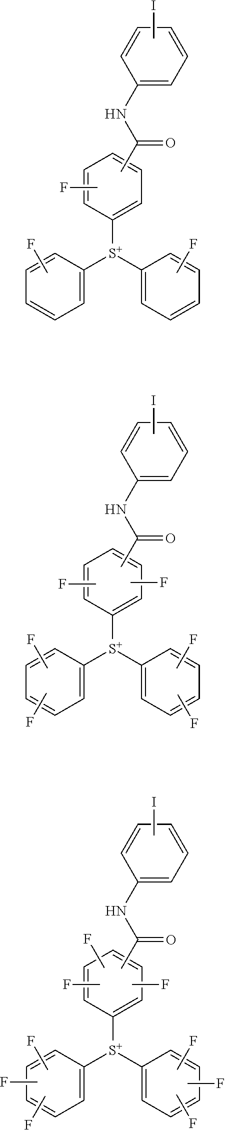

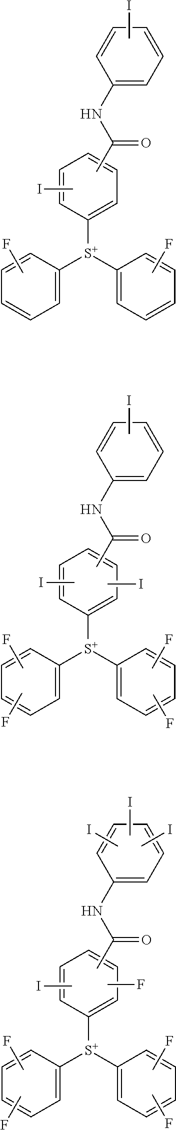

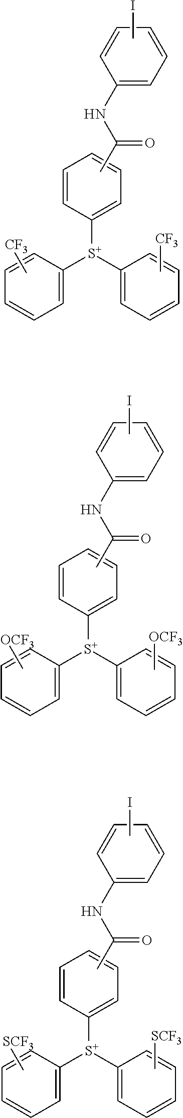

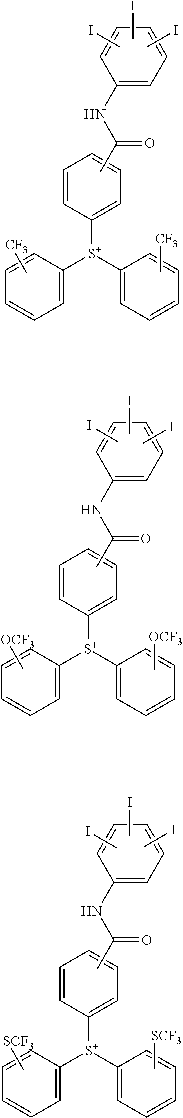

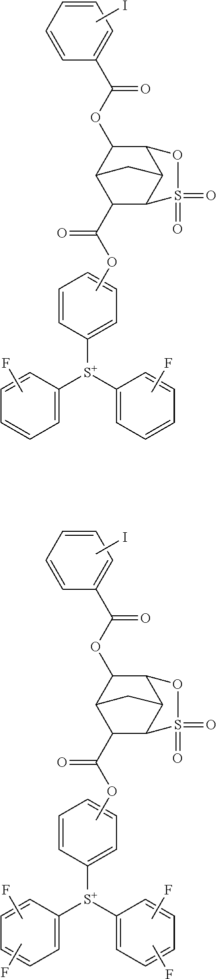

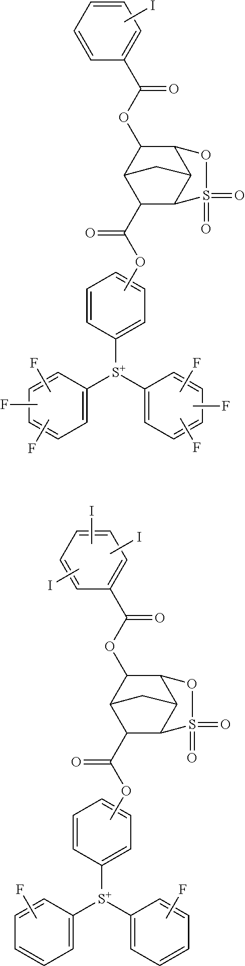

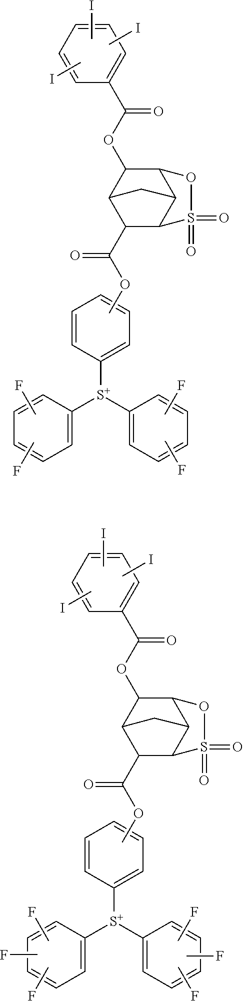

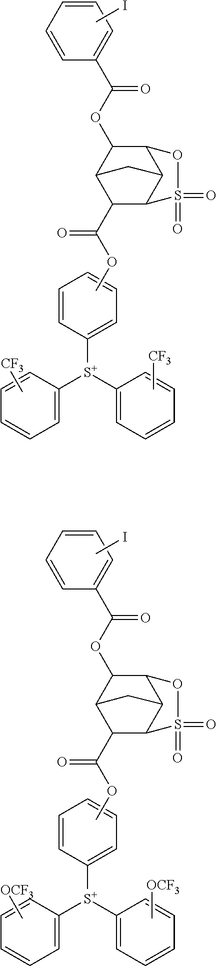

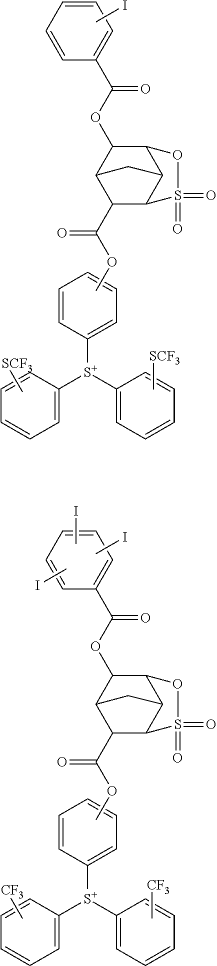

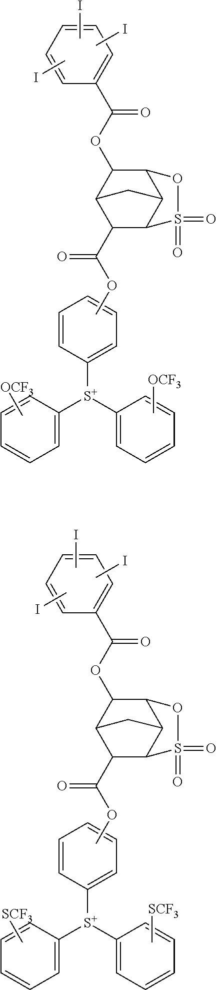

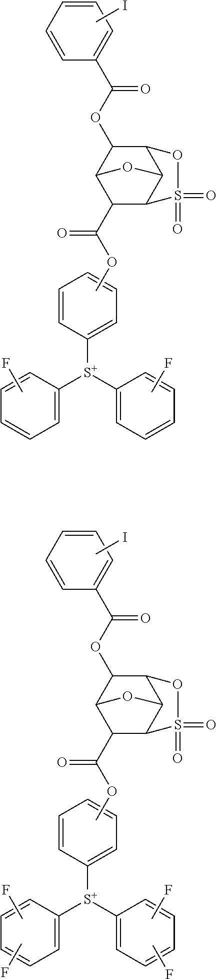

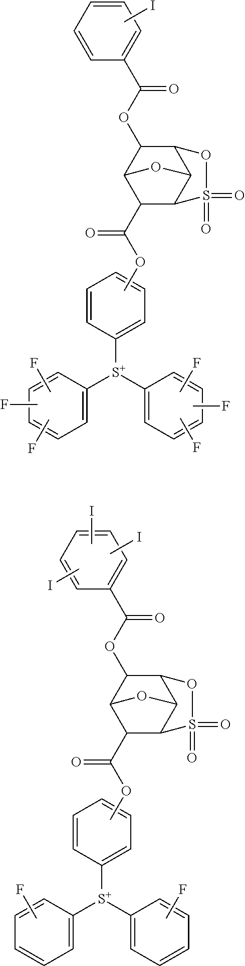

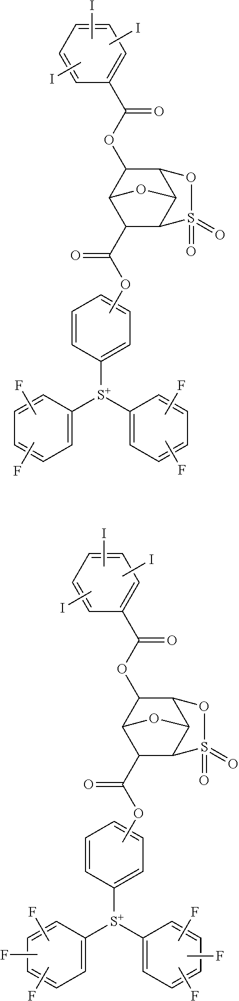

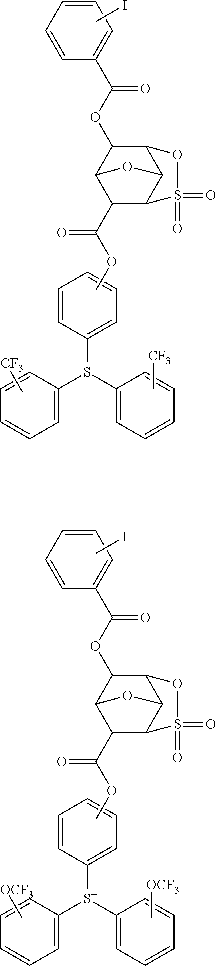

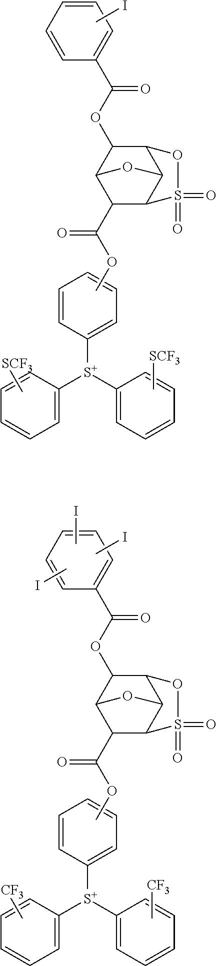

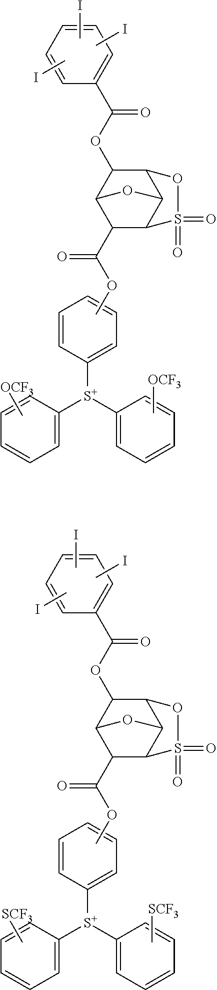

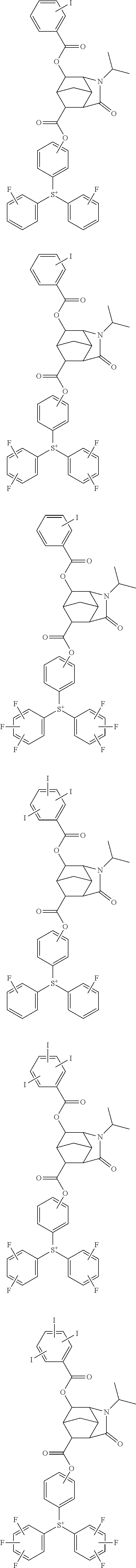

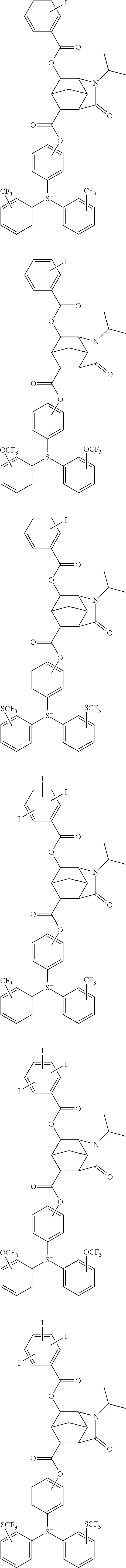

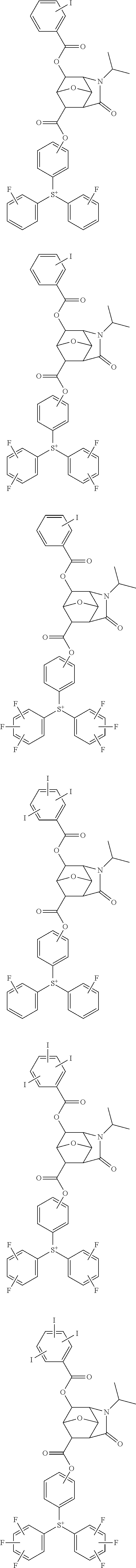

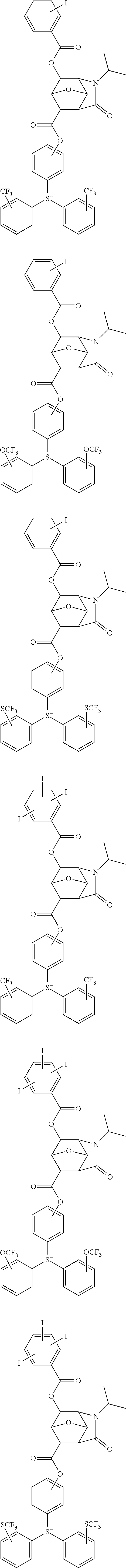

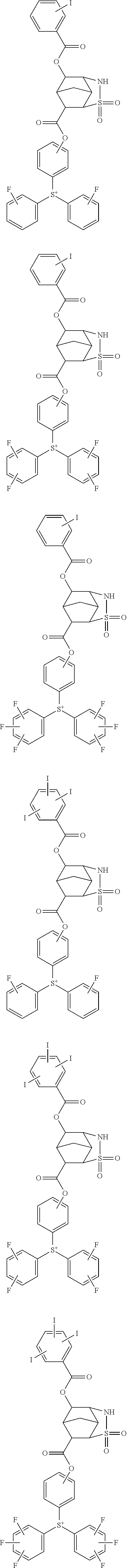

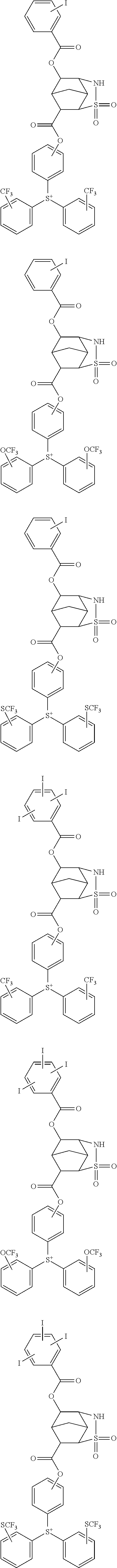

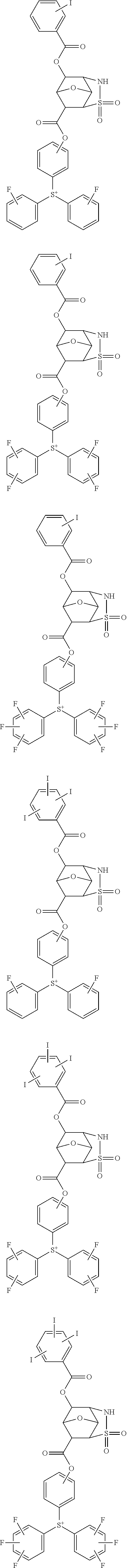

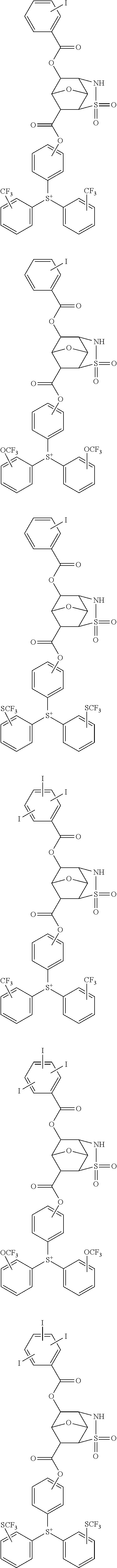

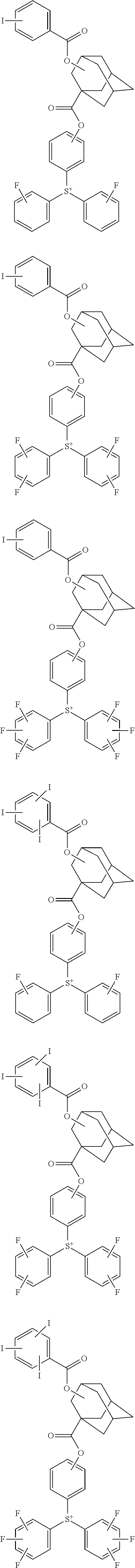

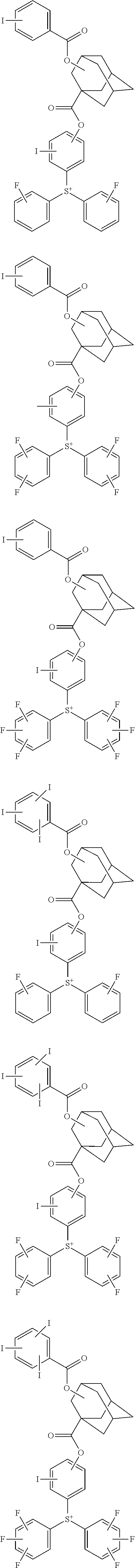

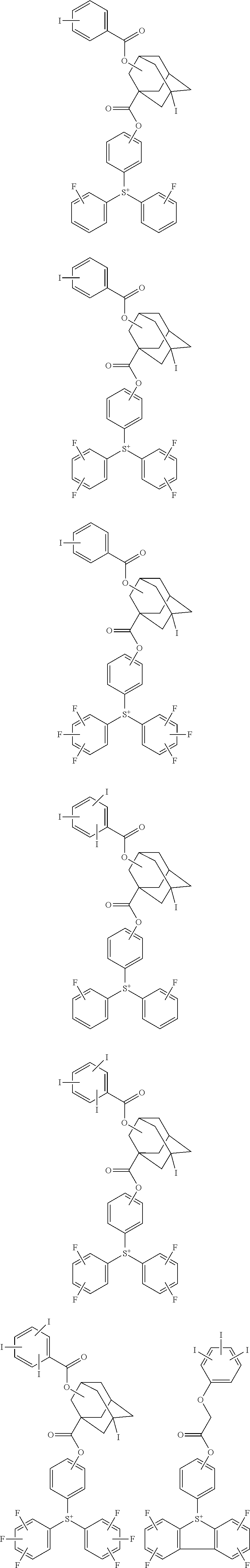

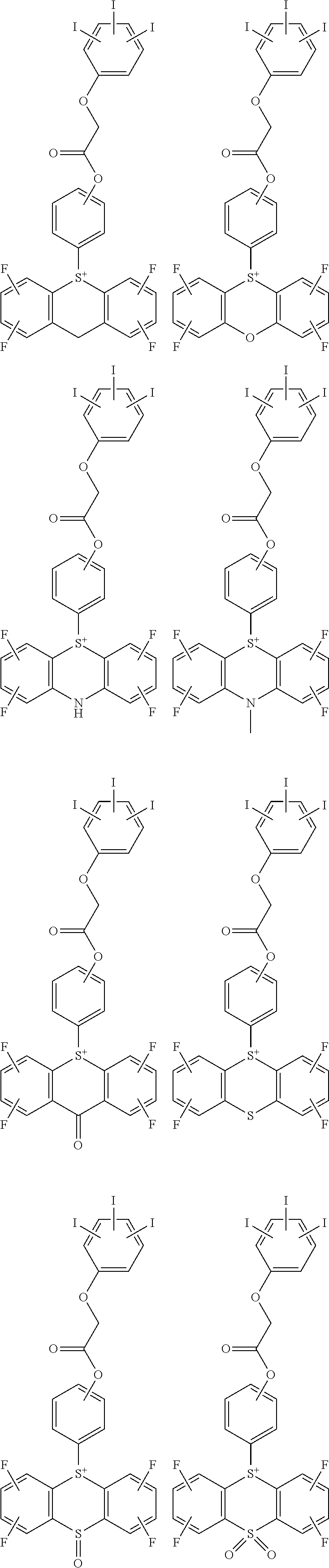

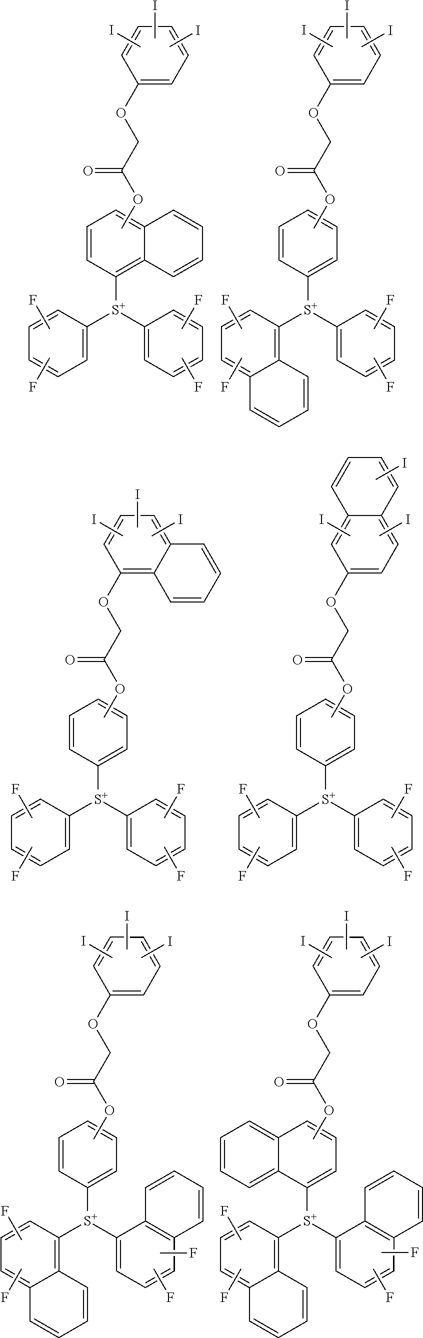

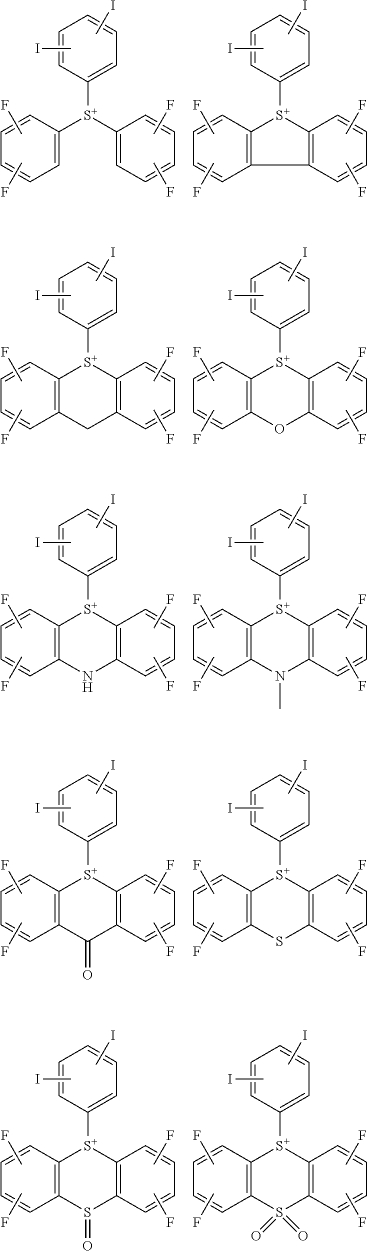

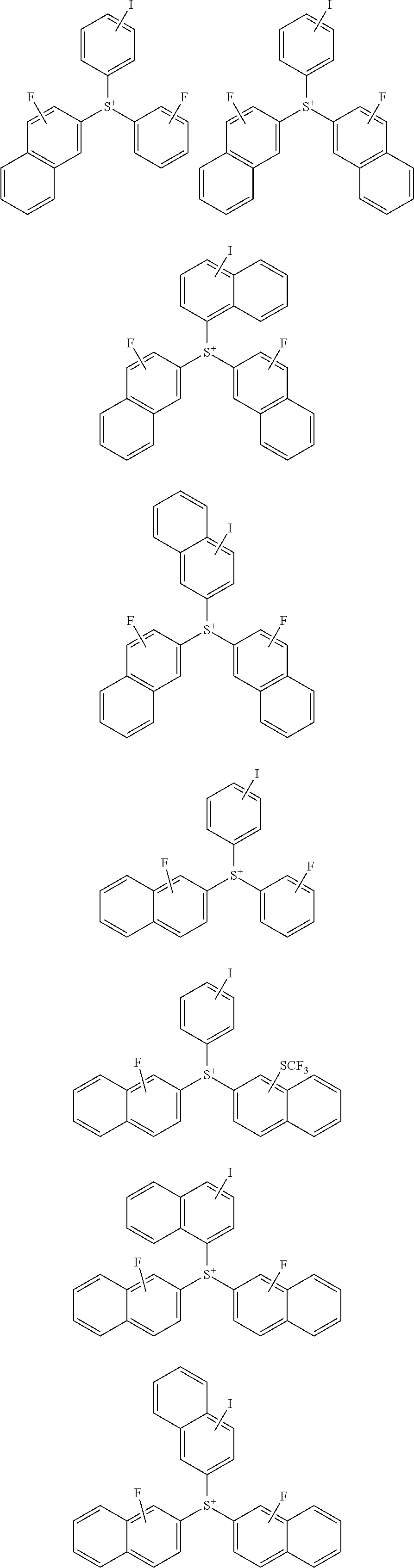

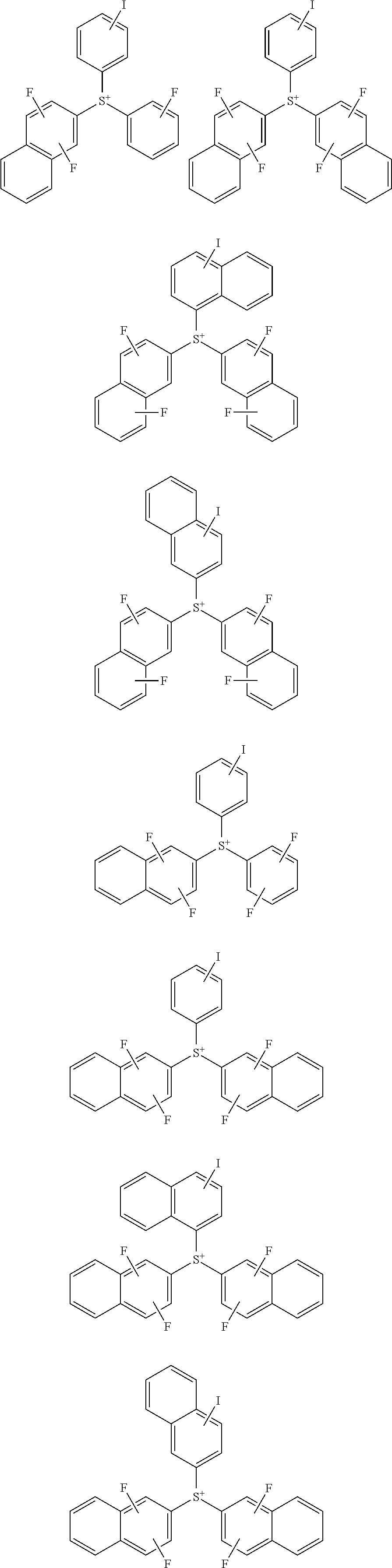

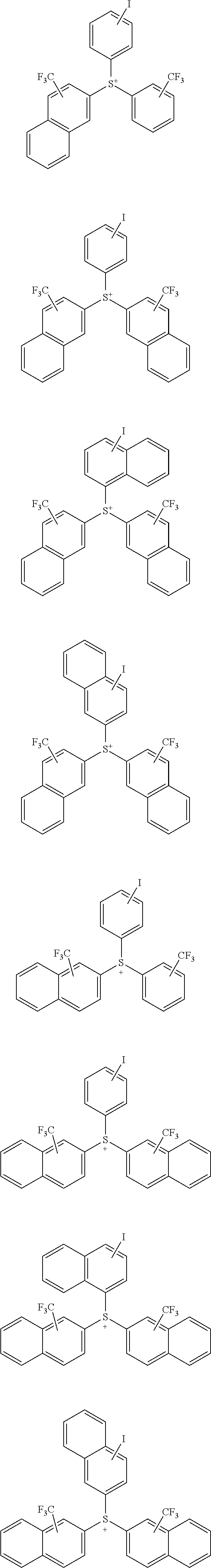

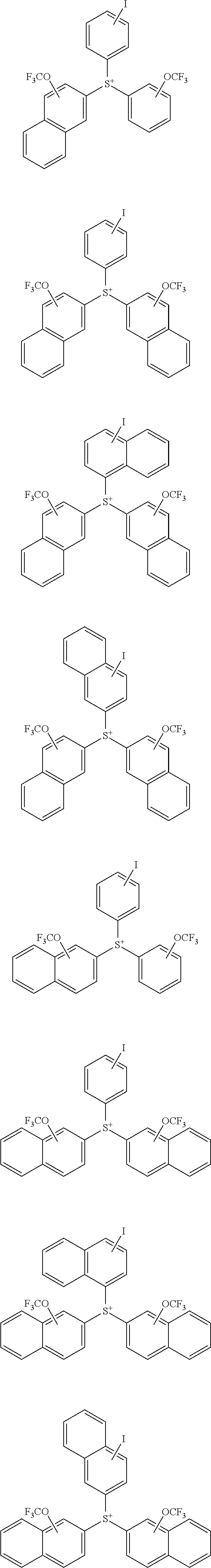

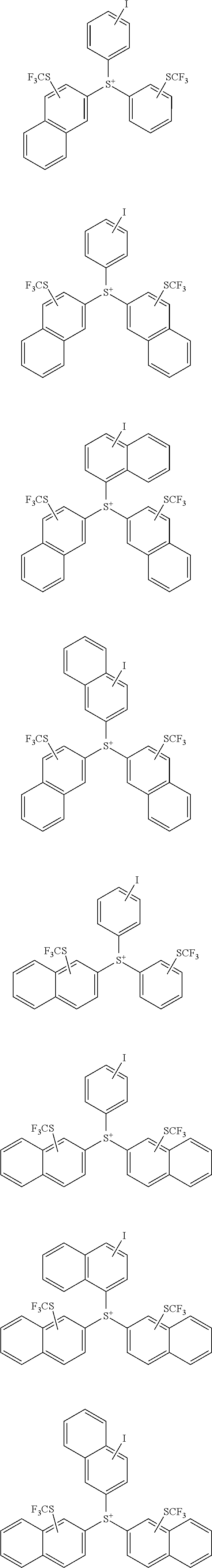

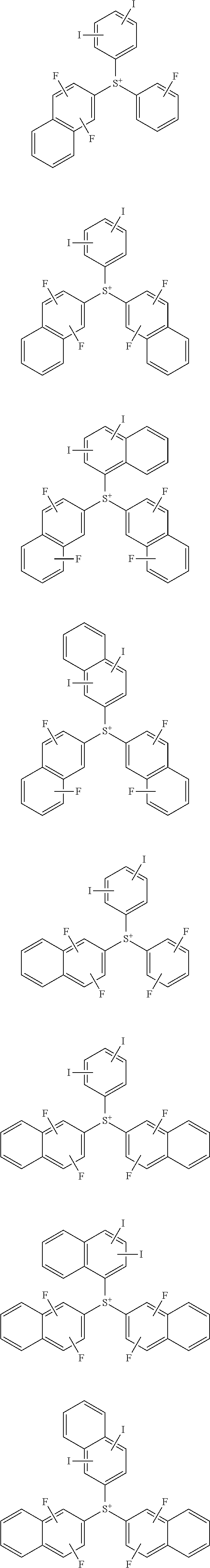

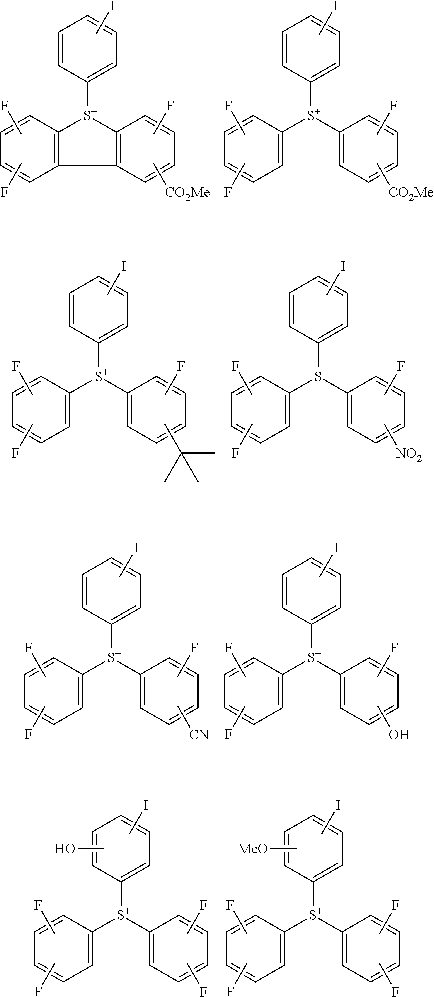

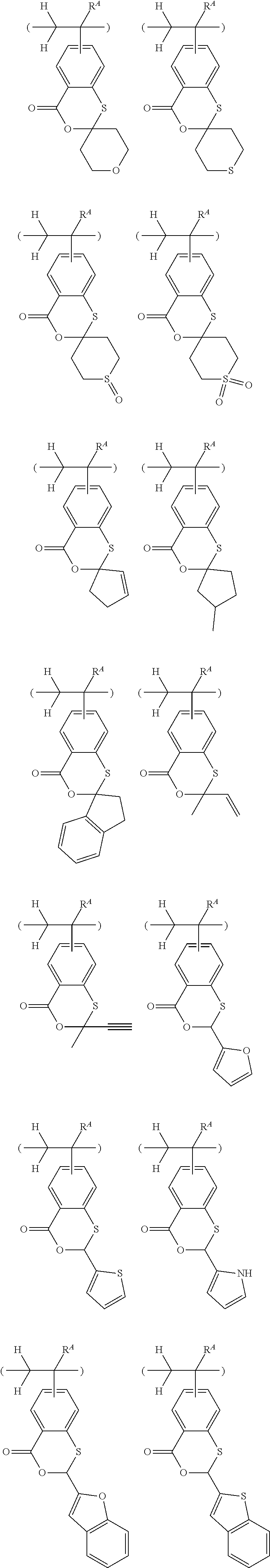

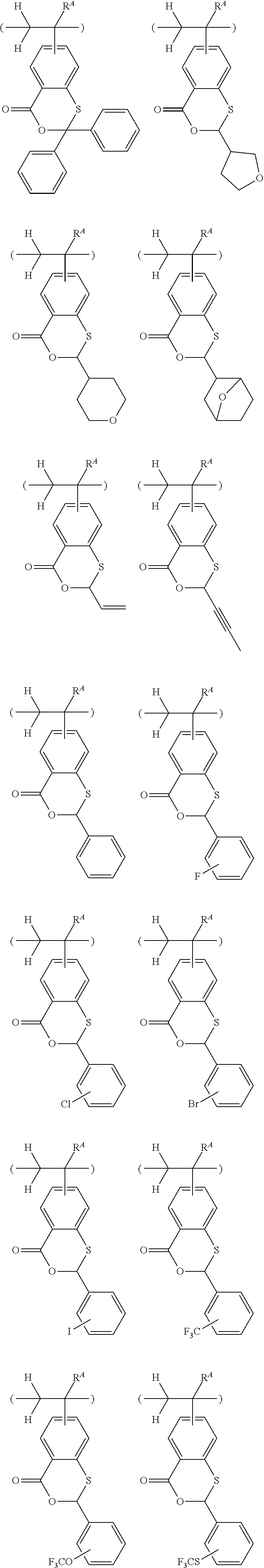

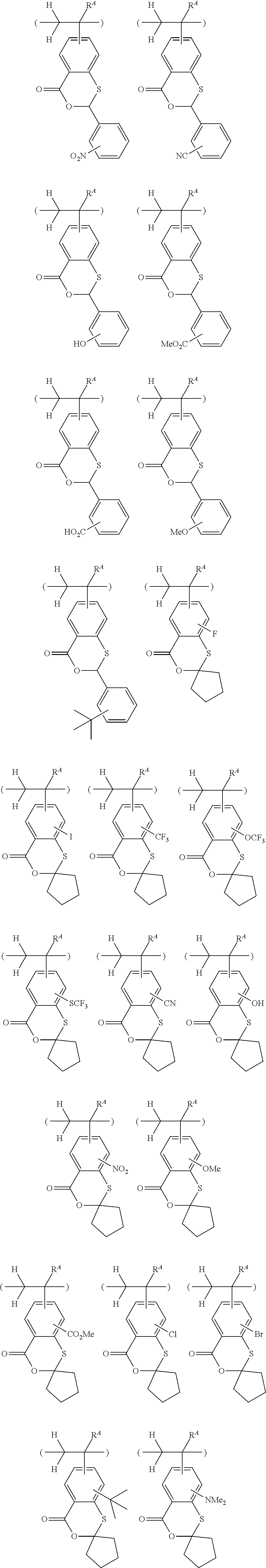

Examples of the onium salt monomer include arbitrary combinations of the anion with the cation, both as exemplified above.

The onium salt monomer may be synthesized, for example, by the same method as the synthesis of the sulfonium salt having a polymerizable anion described in U.S. Pat. No. 8,057,985 (JP 5201363). The synthesis method is not limited thereto.

### [Polymer]

In another aspect, the invention provides a polymer comprising repeat units derived from the onium salt monomer consisting of the anion having formula (a1) and the cation having formula (a2), also referred to as repeat units (a).

In a chemically amplified resist composition, the polymer is a polymer-bound photoacid generator which functions as both a photoacid generator and a base polymer. When the triaryl sulfonium cation containing iodine and fluorine atoms is processed by the EUV lithography of wavelength 13.5 nm, iodine which is outstandingly absorptive to EUV generates secondary electrons during EUV exposure. Also, the electron withdrawing effect of fluorine serves to lower the energy level of the LUMO of the frontier orbital theory so that the cation is more likely to accept the generated secondary electrons, whereby the decomposition of the cation is promoted and the acid is effectively generated. In the fluorosulfonic acid anion containing an aromatic ring structure substituted with a polymerizable group and iodine, on the other hand, the anion generates secondary electrons because the anion also contains iodine, whereby the decomposition of the cation is promoted. The synergy of these effects results in a higher sensitivity. Since iodine has a large atomic weight and the generated acid is bound to the polymer backbone, the advantage of reduced acid diffusion is obtained. Since the polymerizable group in the form of a styrene or vinylnaphthalene structure is more robust than the conventional polymerizable group in the form of a methacrylate, the polymer has a higher glass transition temperature (Tg). Owing to the interaction of aromatic rings within or between the polymer (π-π stacking effect), the polymer is arranged in order. This ensures that when a resist film is developed in a developer to form a small-size pattern, the resist pattern is resistant to collapse. Also, in the etching step after the small-size pattern formation, the aromatic ring directly bonded to the backbone contributes to better etching resistance. In the iodized aromatic ring in formula (a1), iodine is preferably attached at the ortho position to the carbon atom to which L^{B1} is attached. It is expected that this attachment restrains the rotation of the bond axis between the aromatic ring to which the polymerizable group is attached and the aromatic ring which is substituted with iodine. Due to the synergy of these effects, the inventive polymer is effective for suppressing the excessive diffusion of the generated acid and enables to form line patterns with reduced LWR and hole patterns with improved CDU, both the patterns having collapse resistance. The inventive polymer is thus useful as one component of a chemically amplified resist composition, especially of positive tone.

The polymer may further comprise repeat units having the formula (b1) or repeat units having the formula (b2). These units are also referred to as repeat units (b1) and (b2).

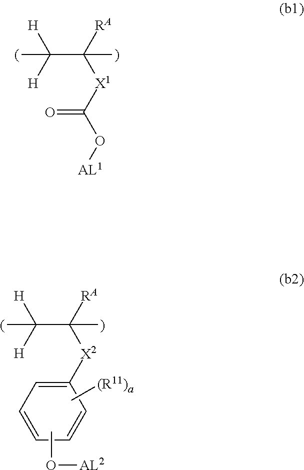

In formulae (b1) and (b2), R^{A} is each independently hydrogen, fluorine, methyl or trifluoromethyl.

In formula (b1), X¹ is a single bond, phenylene group, naphthylene group, *—C(═O)—O—X¹¹— or *—C(═O)—NH—X¹¹— wherein * designates a point of attachment to the carbon atom in the backbone. The phenylene and naphthylene groups may be substituted with hydroxy, nitro, cyano, an optionally fluorinated C₁-C₁₀ alkoxy moiety or halogen. X¹¹ is a C₁-C₁₀ saturated hydrocarbylene group, phenylene group or naphthylene group, and the saturated hydrocarbylene group may contain hydroxy, ether bond, ester bond or lactone ring.

In formula (b2), X² is a single bond, *—C(═O)—O— or *—C(═O)—NH—, wherein * designates a point of attachment to the carbon atom in the backbone. R¹¹ is halogen, cyano, hydroxy, nitro, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom, and “a” is an integer of 0 to 4, preferably 0 or 1.

In formulae (b1) and (b2), AL¹ and AL² are each independently an acid labile group. The acid labile group may be selected from a variety of such groups, for example, those groups described in JP-A 2013-080033 (U.S. Pat. No. 8,574,817) and JP-A 2013-083821 (U.S. Pat. No. 8,846,303).

Typical of the acid labile group are groups of the following formulae (AL-1) to (AL-3).

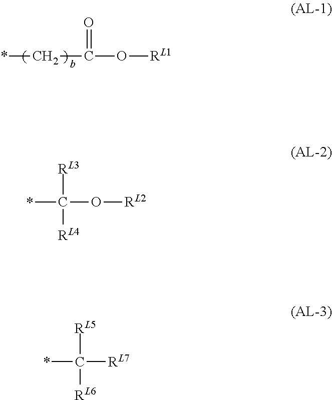

In formulae (AL-1) and (AL-2), R^{L1} and R^{L2} are each independently a C₁-C₄₀ hydrocarbyl group which may contain a heteroatom such as oxygen, sulfur, nitrogen, fluorine or iodine. The hydrocarbyl group may be straight, branched or cyclic. Inter alia, C₁-C₂₀ hydrocarbyl groups are preferred.

In formula (AL-1), b is an integer of 0 to 10, preferably 1 to 5.

In formula (AL-2), R^{L3} and RN are each independently hydrogen or a C₁-C₂₀ hydrocarbyl group which may contain a heteroatom such as oxygen, sulfur, nitrogen, fluorine or iodine. The hydrocarbyl group may be straight, branched or cyclic. Any two of R^{L2}, R^{L3} and R^{L} may bond together to form a C₃-C₂₀ ring with the carbon atom or carbon and oxygen atoms to which they are attached. The ring preferably contains 4 to 16 carbon atoms and is typically alicyclic.

In formula (AL-3), R^{L5}, R^{L6} and R^{L7} are each independently a C₁-C₂₀ hydrocarbyl group which may contain a heteroatom such as oxygen, sulfur, nitrogen, fluorine or iodine. The hydrocarbyl group may be straight, branched or cyclic. Any two of R^{L5}, R^{L6} and R^{L7} may bond together to form a C₃-C₂₀ ring with the carbon atom to which they are attached. The ring preferably contains 4 to 16 carbon atoms and is typically alicyclic.

Examples of the repeat unit (b1) are shown below, but not limited thereto. Herein R^{A} and AL¹ are as defined above.

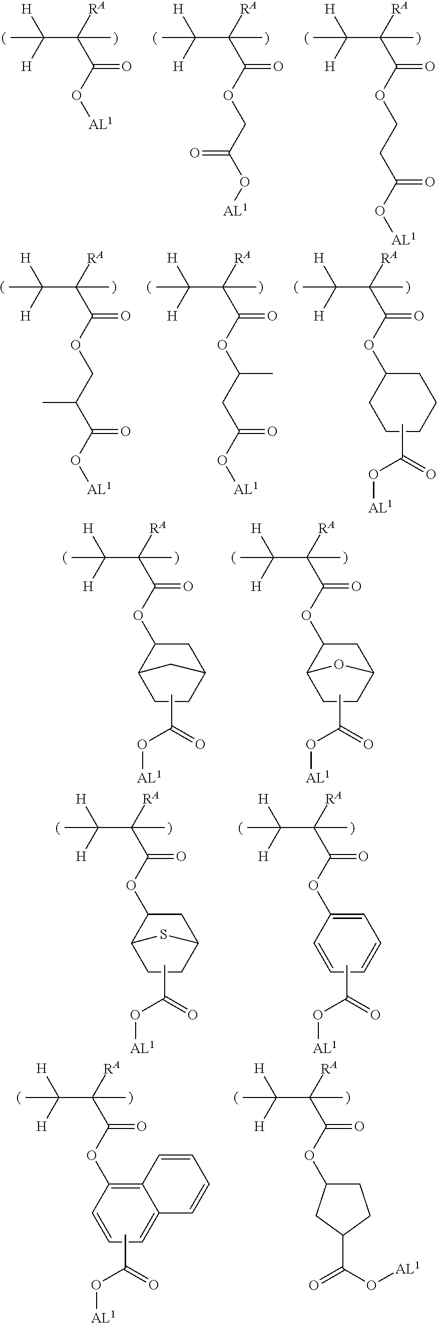

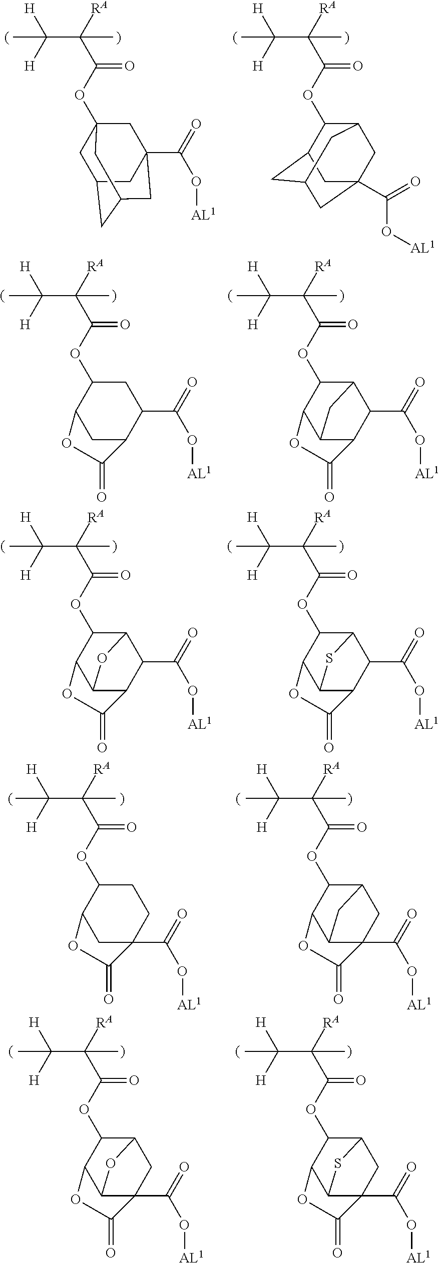

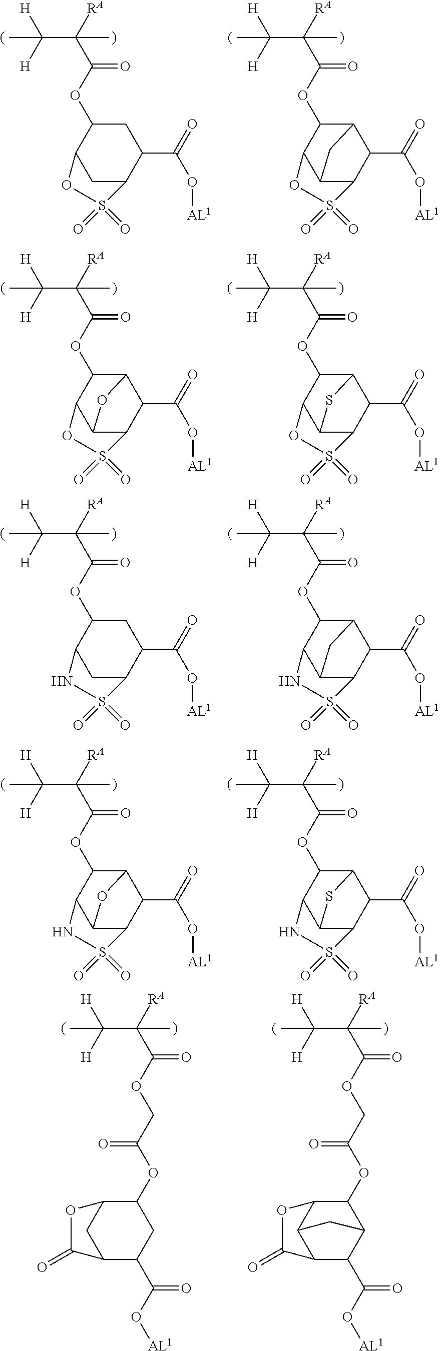

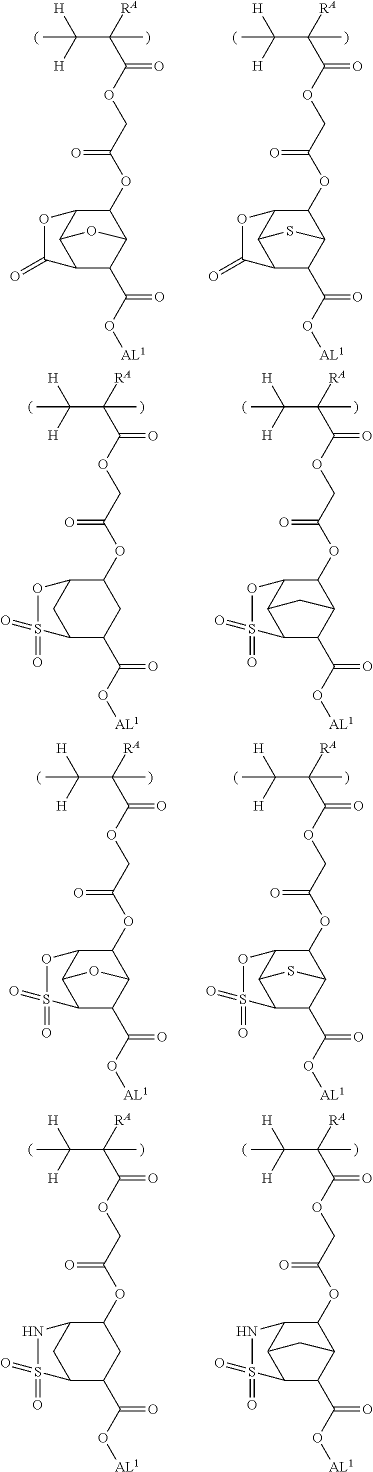

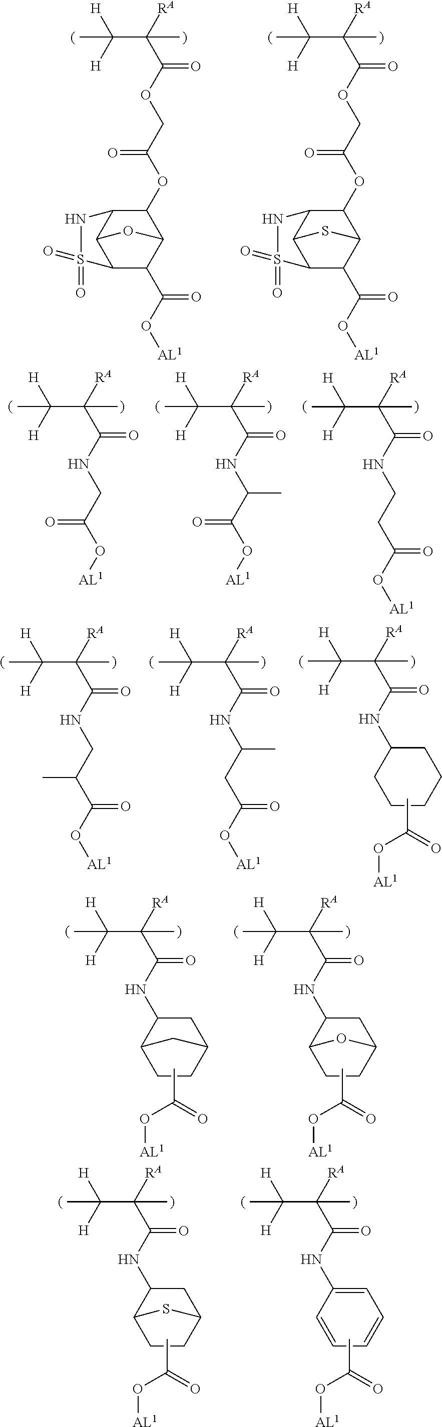

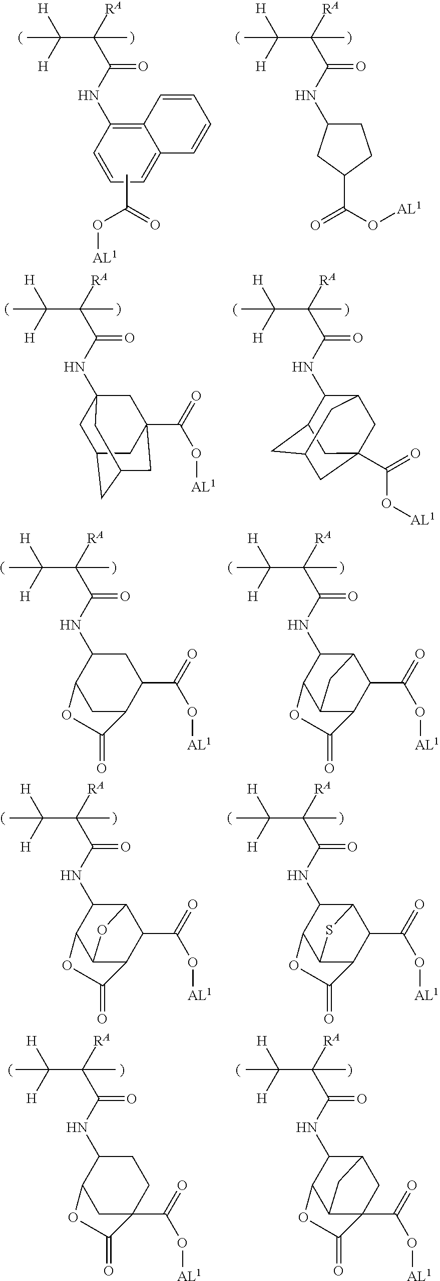

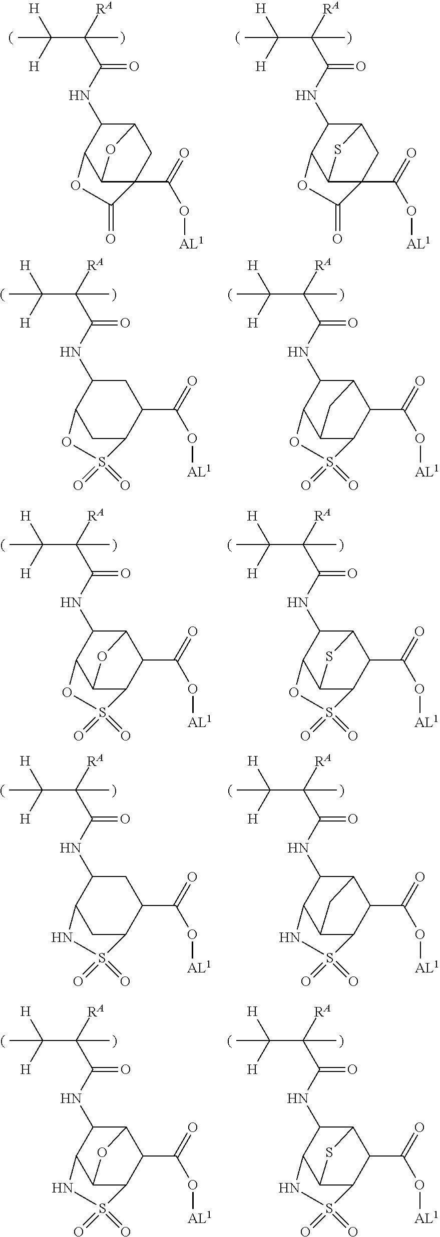

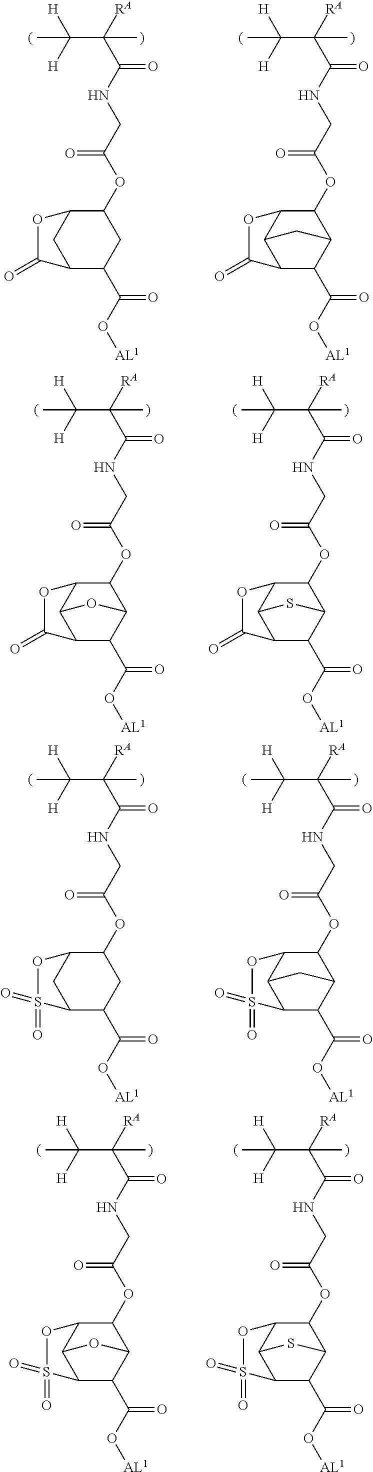

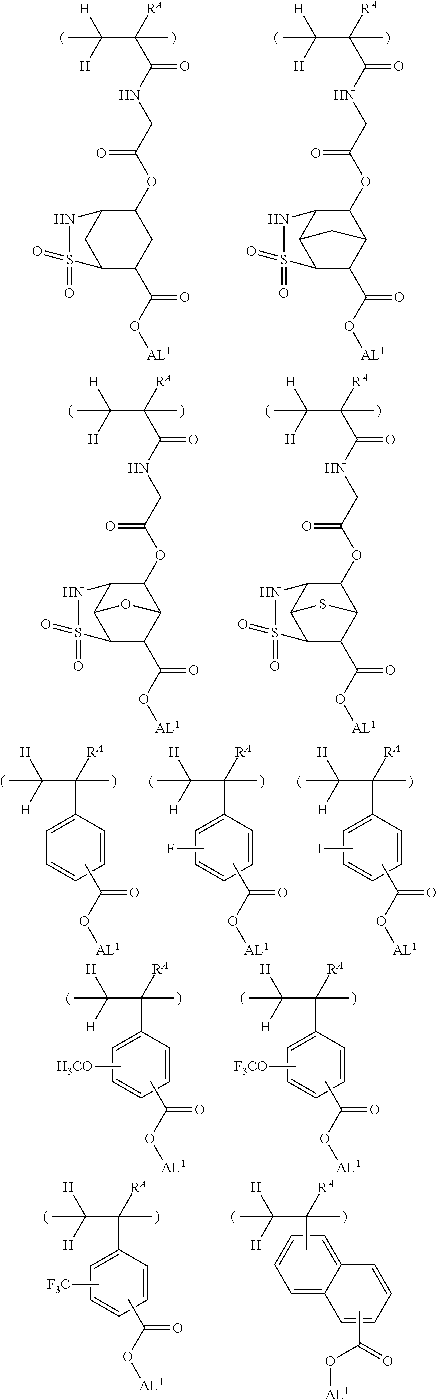

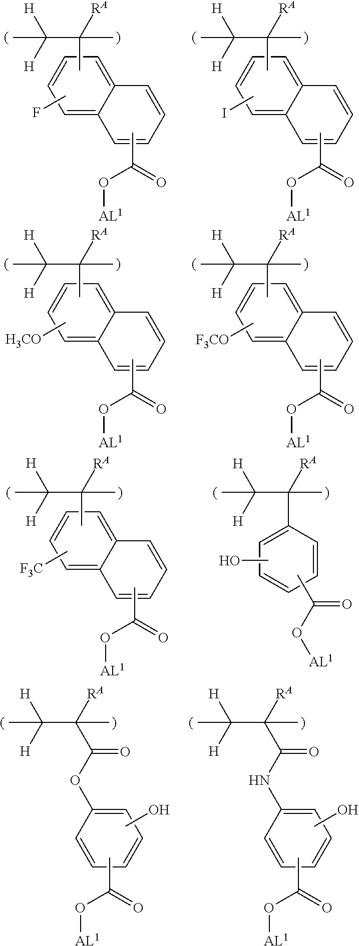

Examples of the repeat unit (b2) are shown below, but not limited thereto. Herein R^{A} and AL² are as defined above.

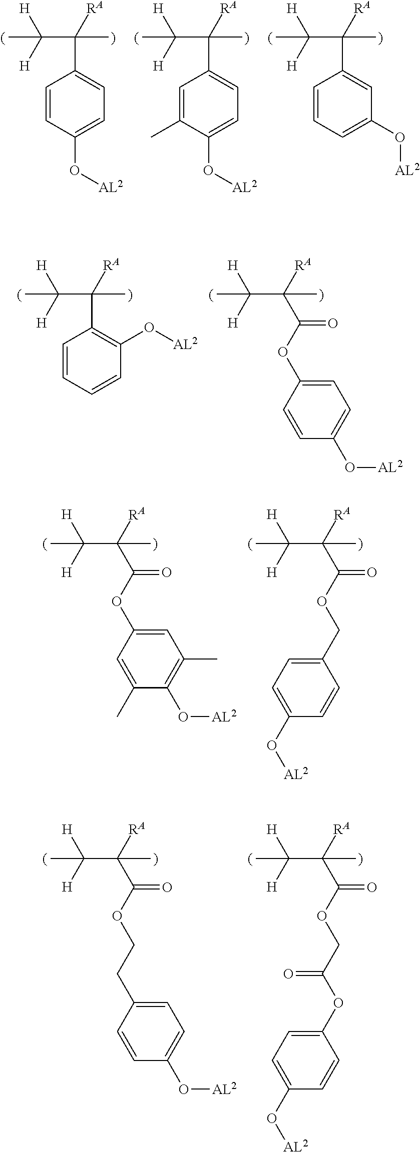

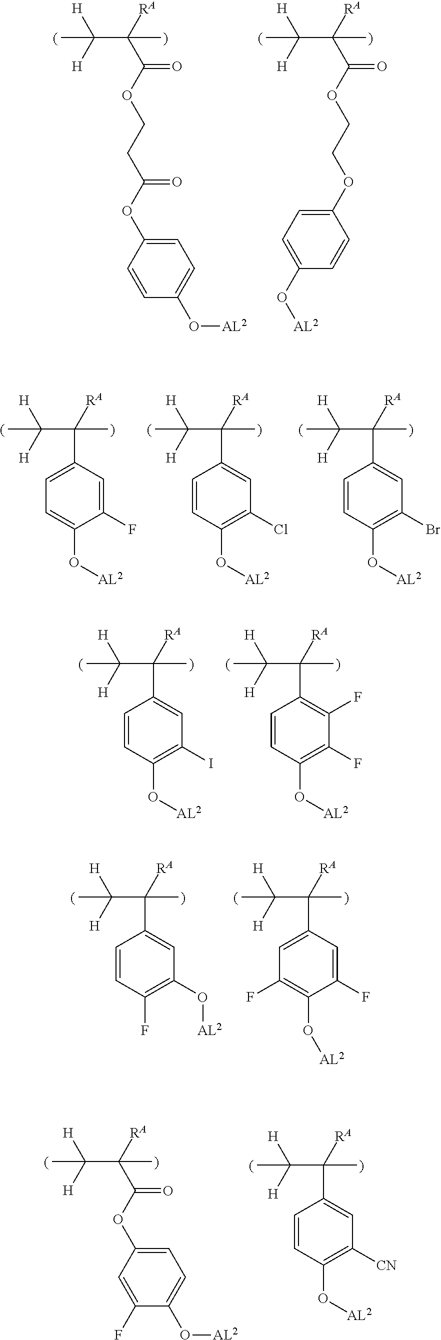

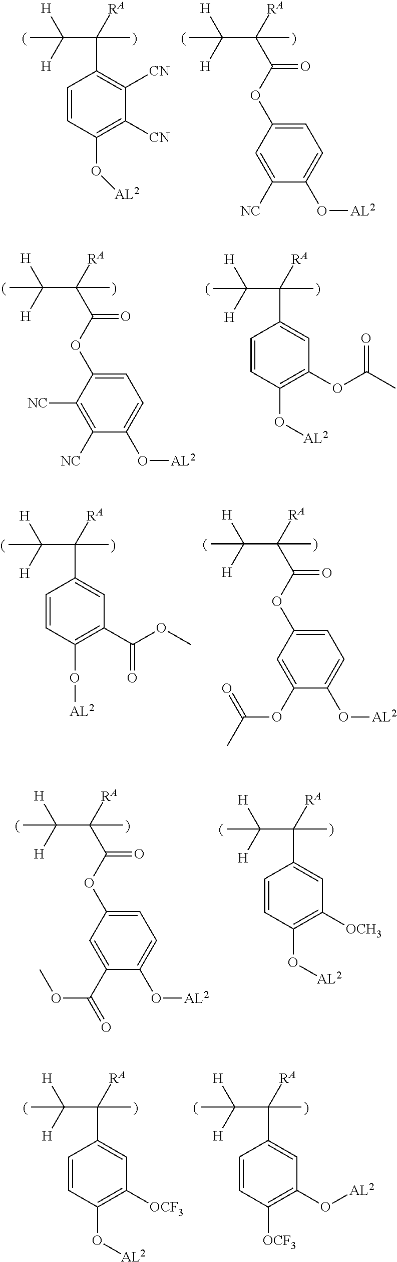

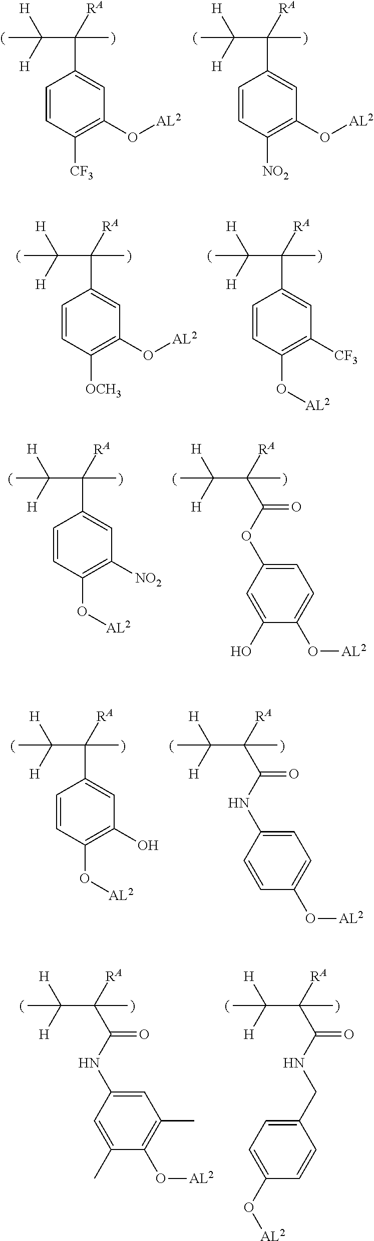

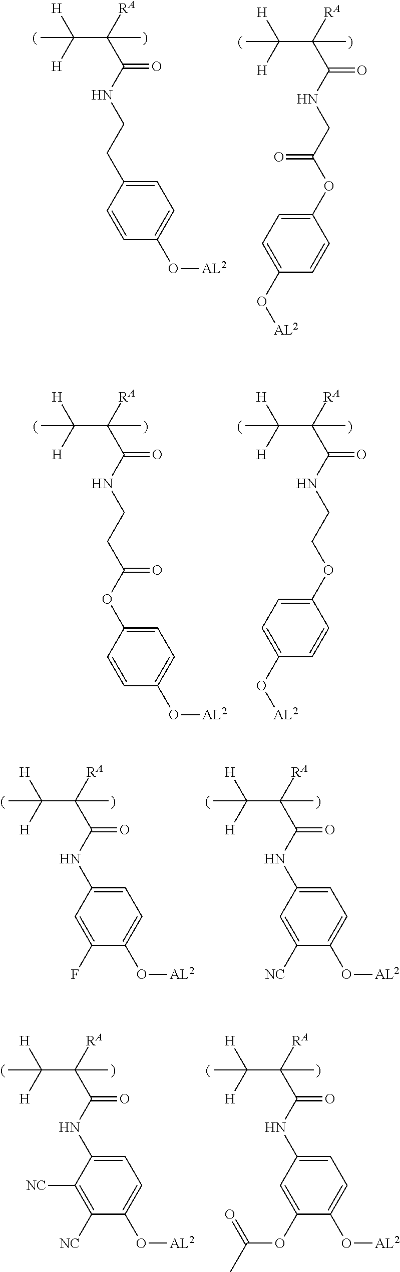

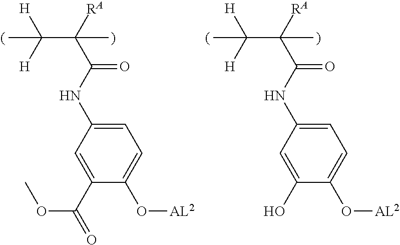

The polymer may further comprise repeat units having the formula (b3), which are also referred to as repeat units (b3).

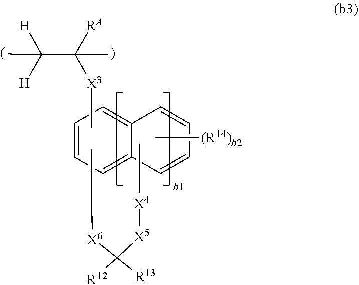

In formula (b3), b1 is 0 or 1. The relevant structure is a benzene ring in case of b1=0 and a naphthalene ring in case of b1=1. From the aspect of solvent solubility, the benzene ring corresponding to b1=0 is preferred. The subscript b2 is an integer of 0 to 3 when b1=0 and an integer of 0 to 5 when b1=1. It is preferred from the aspect of reactant availability that b2 be 0, 1, 2 or 3, more preferably 0, 1 or 2.

In formula (b3), R^{A} is hydrogen, fluorine, methyl or trifluoromethyl, preferably hydrogen or methyl, most preferably hydrogen.

In formula (b3), X³ is a single bond, *—C(═O)—O— or *—C(═O)—NH—, wherein * designates a point of attachment to the carbon atom in the backbone. X³ is preferably a single bond or *—C(═O)—O—, more preferably a single bond.

In formula (b3), R¹² and R¹³ are each independently hydrogen or a C₁-C₂₀ hydrocarbyl group which may contain a heteroatom. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cylopropylmethyl, 4-methylcyclohexyl, cyclohexylmethyl, norbornyl, and adamantyl; C₂-C₂₀ alkenyl groups such as vinyl, allyl, butenyl, and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl, and 2-phenylethyl, and combinations thereof. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety.

Also, R¹² and R¹³ may bond together to form a ring with the carbon atom to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonate ester bond, carbonate bond, lactone ring, sultone ring, carboxylic anhydride (—C(═O)—O—C(═O)—) or haloalkyl moiety.

In formula (b3), R¹⁴ is halogen, hydroxy, cyano, nitro, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{14A})(R^{14B}). R^{14A} and R^{14B} are each independently hydrogen or a C₁-C₆ hydrocarbyl group. Suitable halogen atoms include fluorine, chlorine, bromine and iodine, with fluorine and iodine being preferred. The hydrocarbyl group and hydrocarbyl moiety in the hydrocarbyloxy, hydrocarbyloxycarbonyl, and hydrocarbylthio groups may be saturated or unsaturated and straight, branched or cyclic. Examples thereof are as exemplified above for the hydrocarbyl groups R¹ and R². In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, cyano moiety, fluorine, chlorine, bromine, iodine, carbonyl moiety, ether bond, ester bond, sulfonate ester bond, carbonate bond, lactone ring, sultone ring, carboxylic anhydride (—C(═O)—O—C(═O)—) or haloalkyl moiety. A plurality of R¹⁴ may be identical or different when b2 is 2 or 3.

When b2 is 2 or more, a plurality of R¹⁴ may bond together to form a ring with carbon atoms in the aromatic ring to which they are attached. Examples of the ring include cyclopropane, cyclobutane, cyclopentane, cyclohexane, norbornane, and adamantane rings. In the ring, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the ring may contain a hydroxy moiety, fluorine, chlorine, bromine, iodine, cyano moiety, carbonyl moiety, ether bond, ester bond, sulfonic ester bond, carbonate bond, lactone ring, sultone ring, carboxylic anhydride (—C(═O)—O—C(═O)—) or haloalkyl moiety.

In formula (b3), X⁴ is a single bond, C₁-C₄ aliphatic hydrocarbylene group, carbonyl group, sulfonyl group or a group obtained by combining the foregoing. It is preferred from the aspect of reactant availability that X⁴ be a single bond, carbonyl or sulfonyl. It is more preferred from the aspect of a polar group created after reaction that X⁴ be a single bond or carbonyl.

In formula (b3), X⁵ and X⁶ are each independently oxygen or sulfur, with the proviso that X⁴ and X⁶ are attached to adjacent carbon atoms on the aromatic ring. X⁵ and X⁶ may be the same or different. It is preferred from the aspect of reactivity that X⁵ and X⁶ be both oxygen.

Examples of repeat unit (b3) are shown below, but not limited thereto. Herein R^{A} is as defined above. The positions of attachment of substituent groups on the aromatic ring are interchangeable.

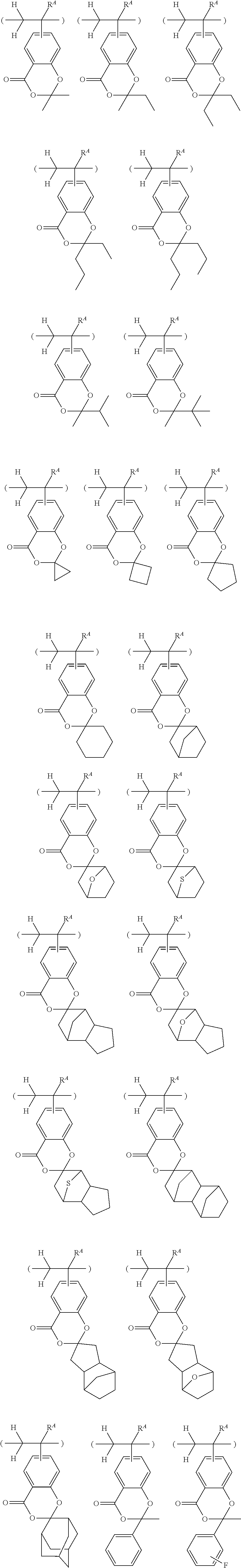

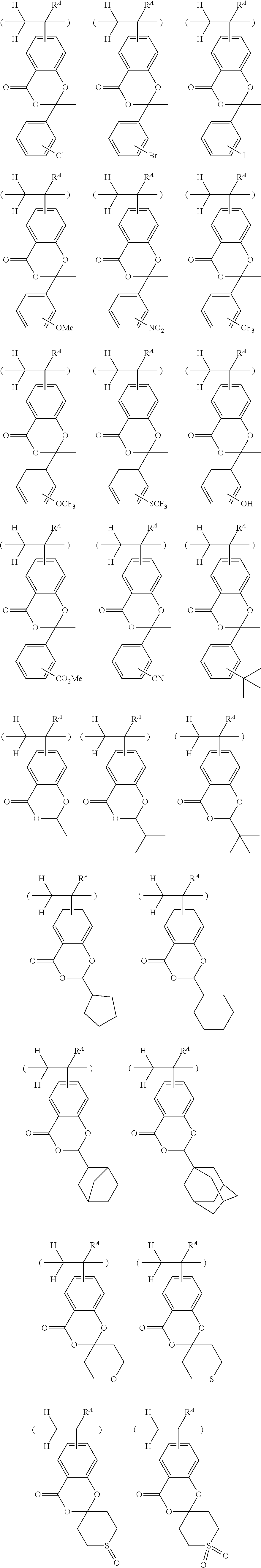

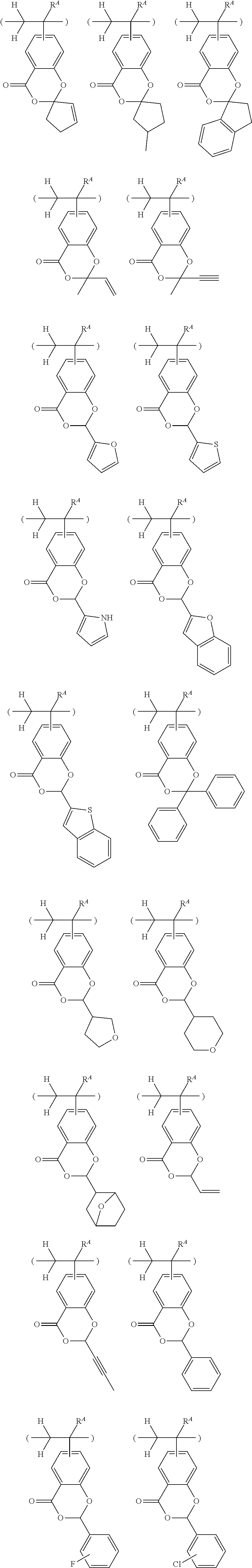

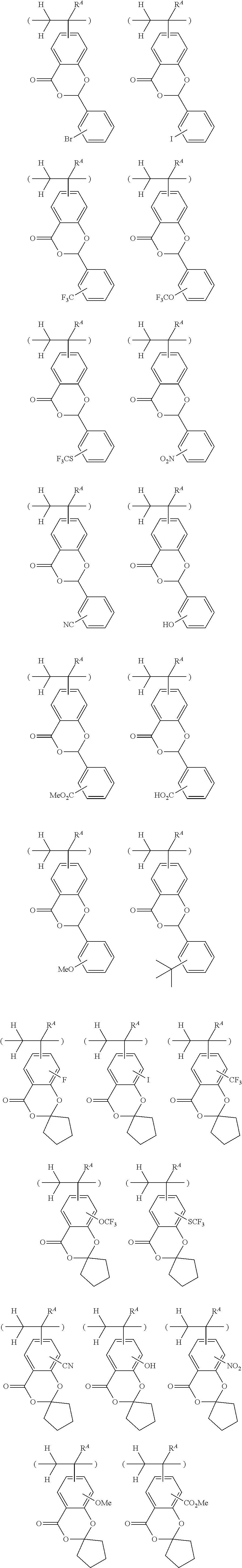

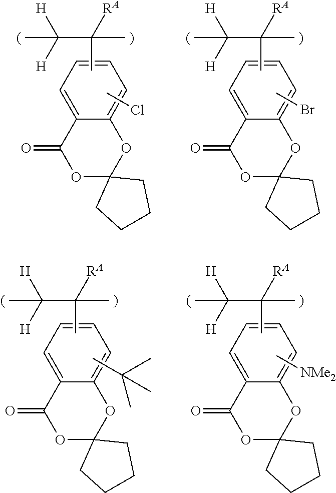

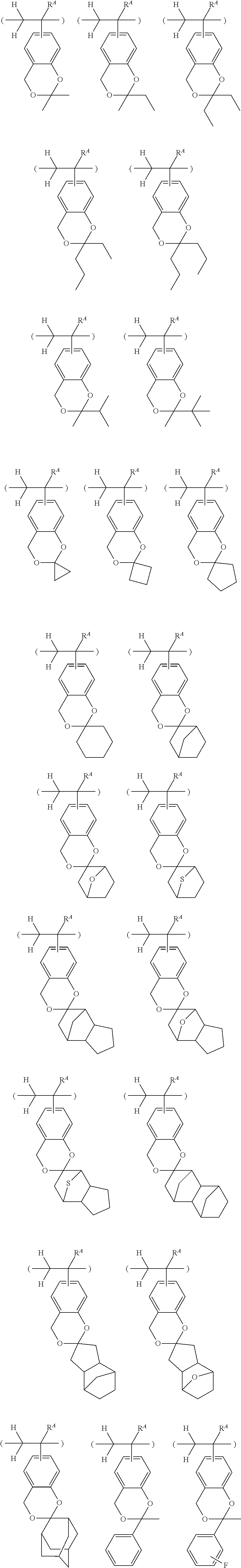

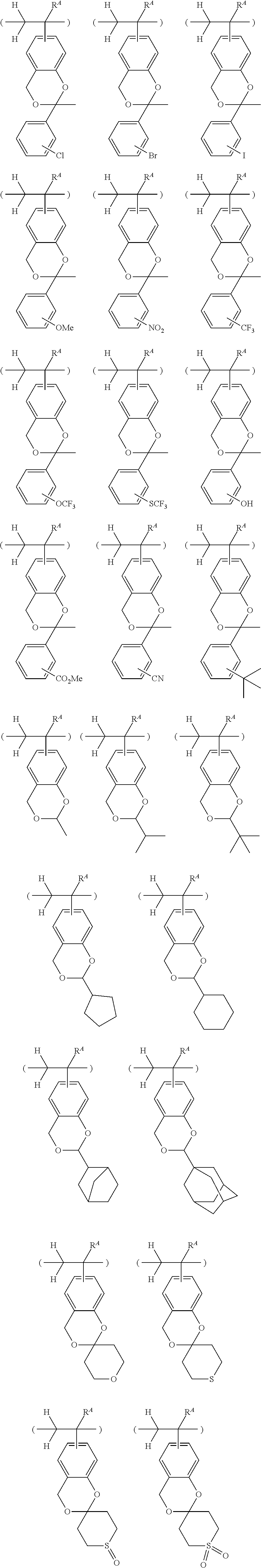

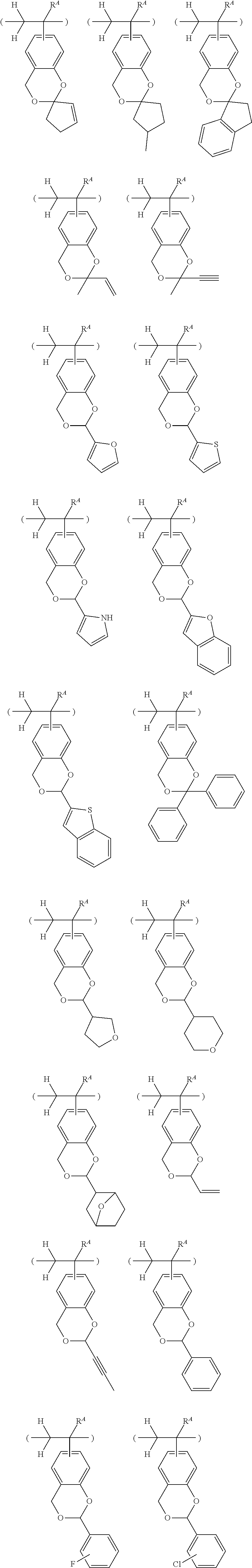

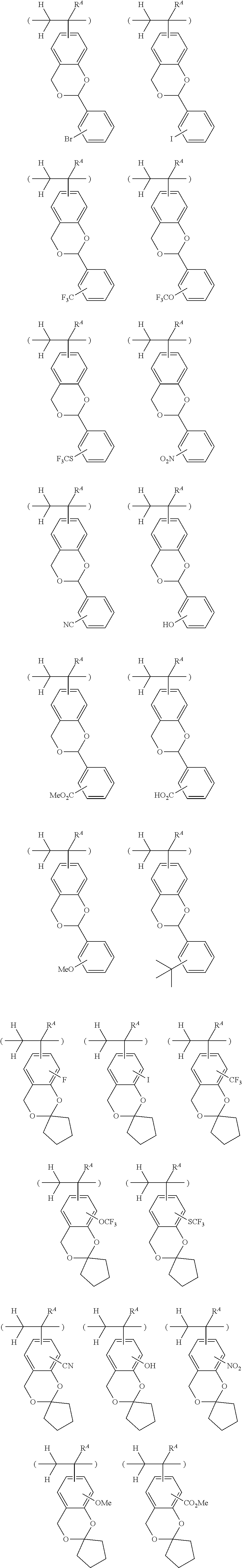

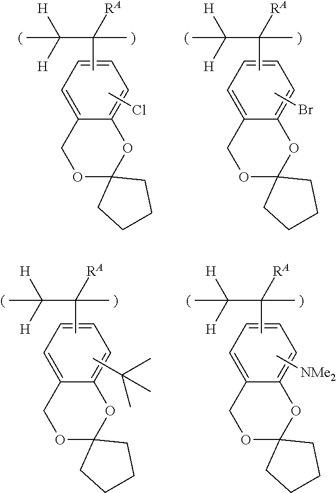

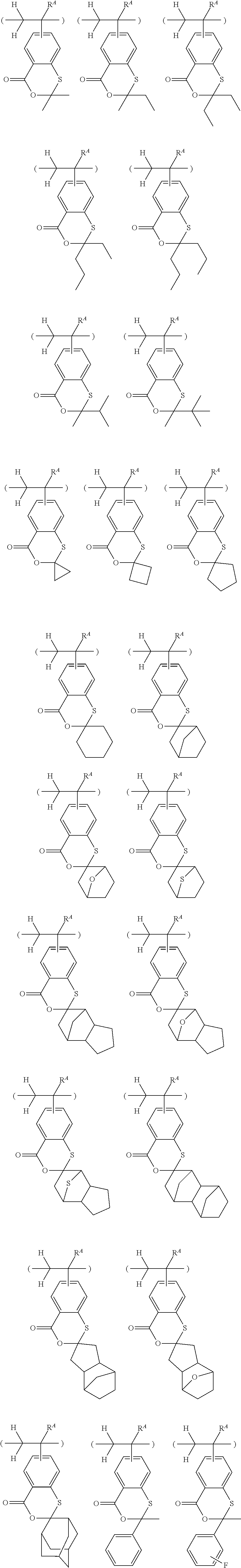

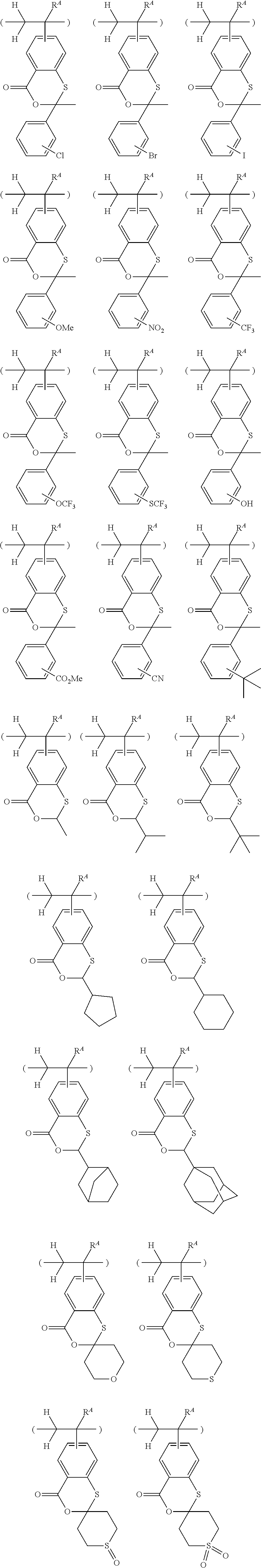

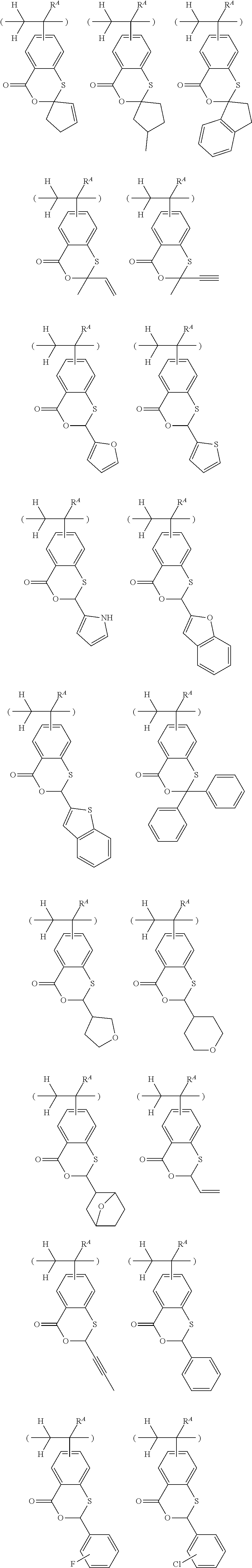

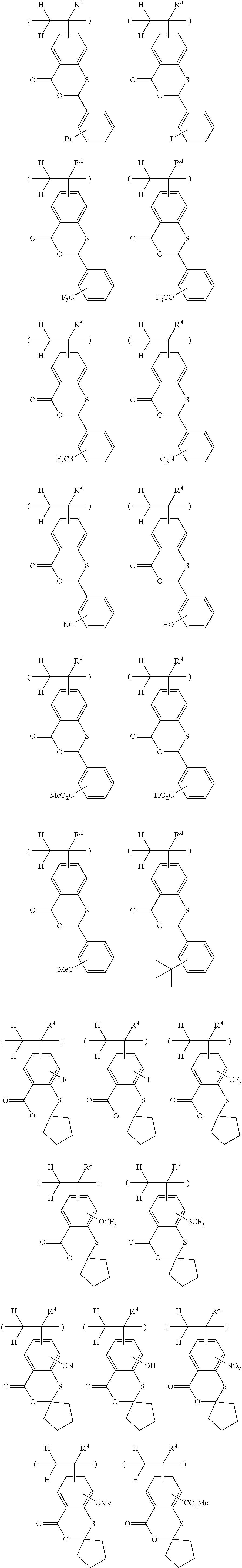

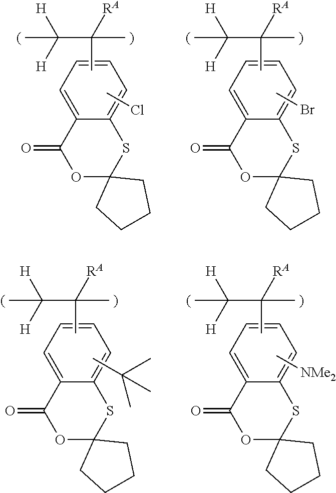

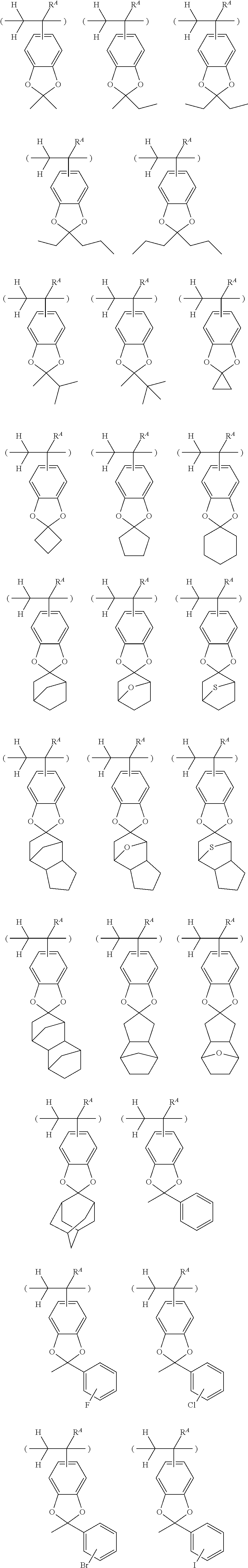

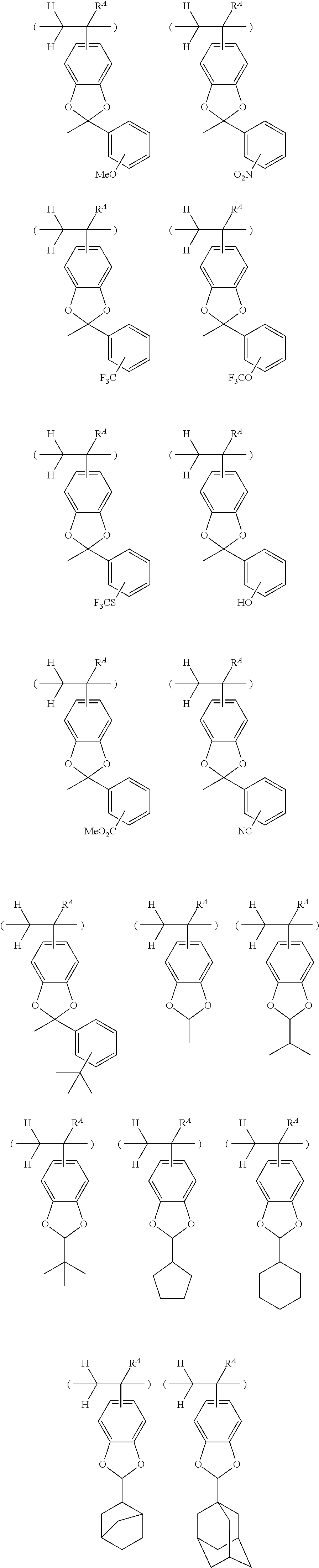

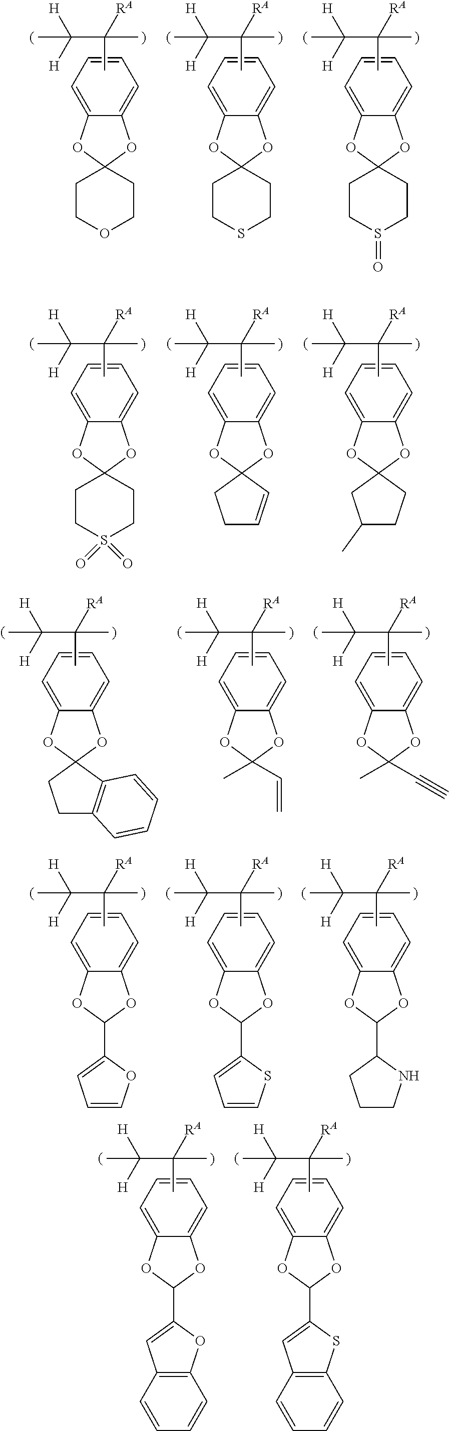

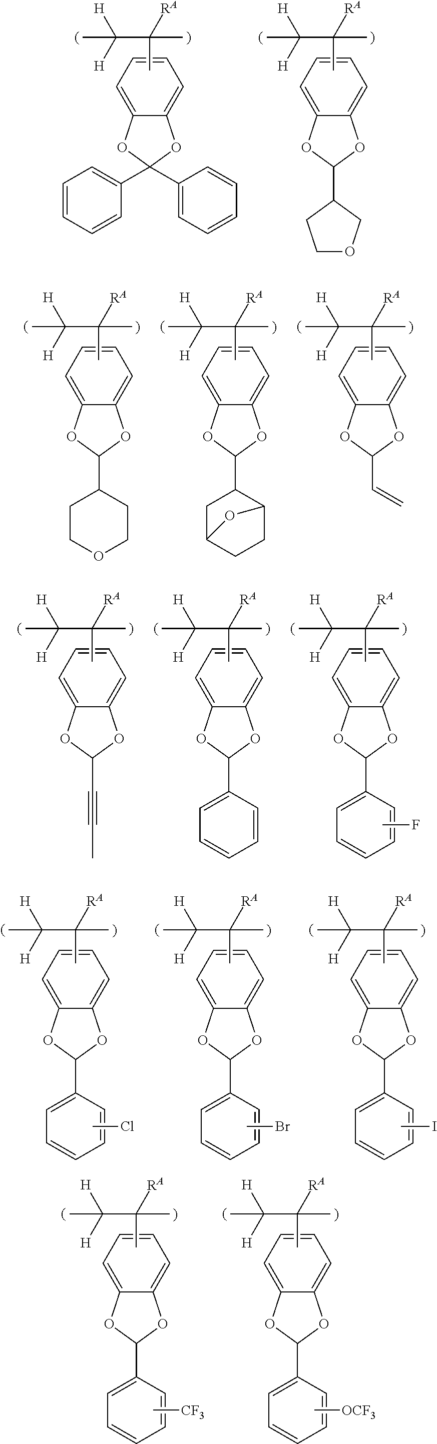

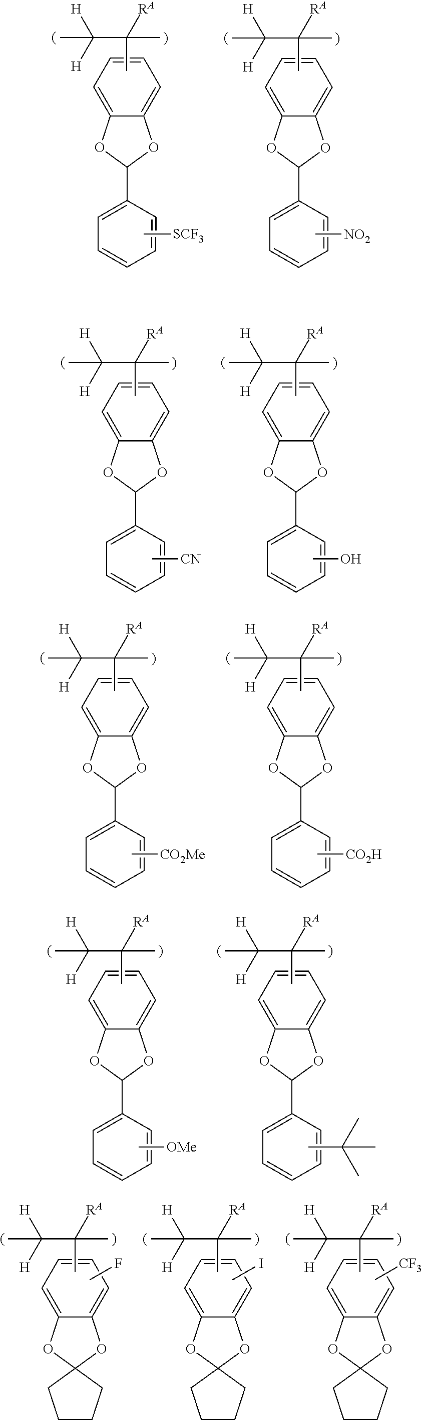

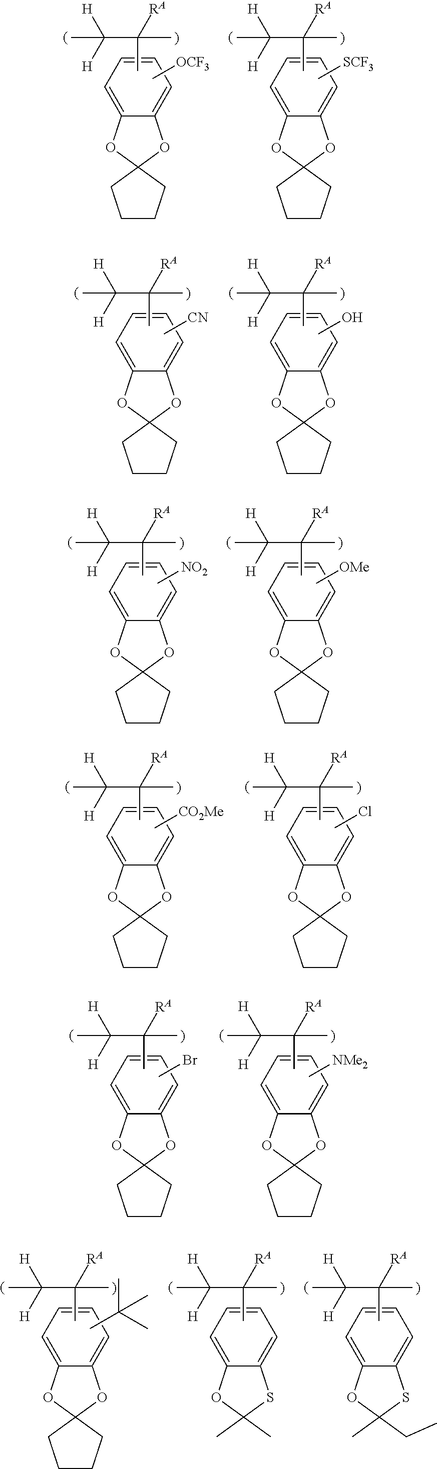

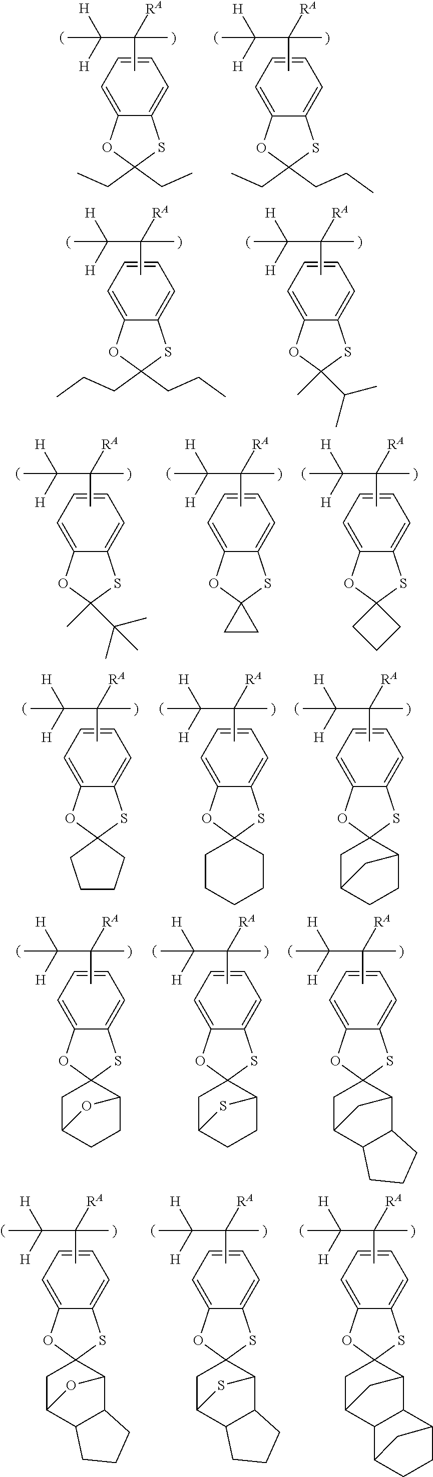

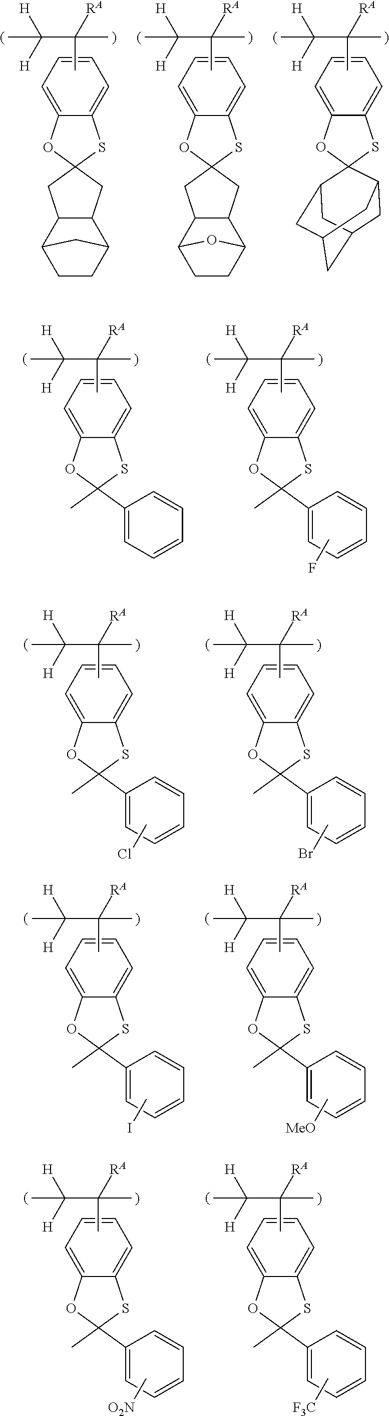

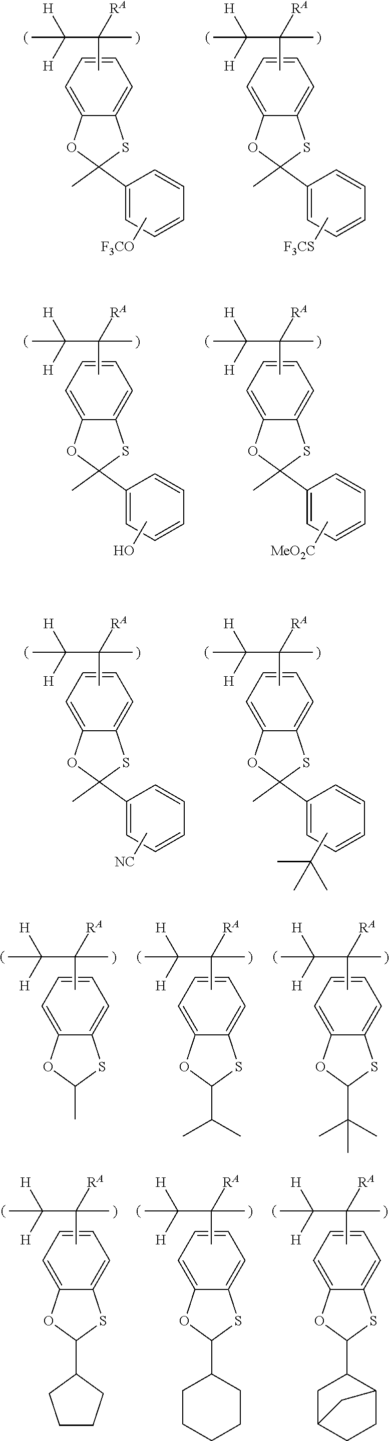

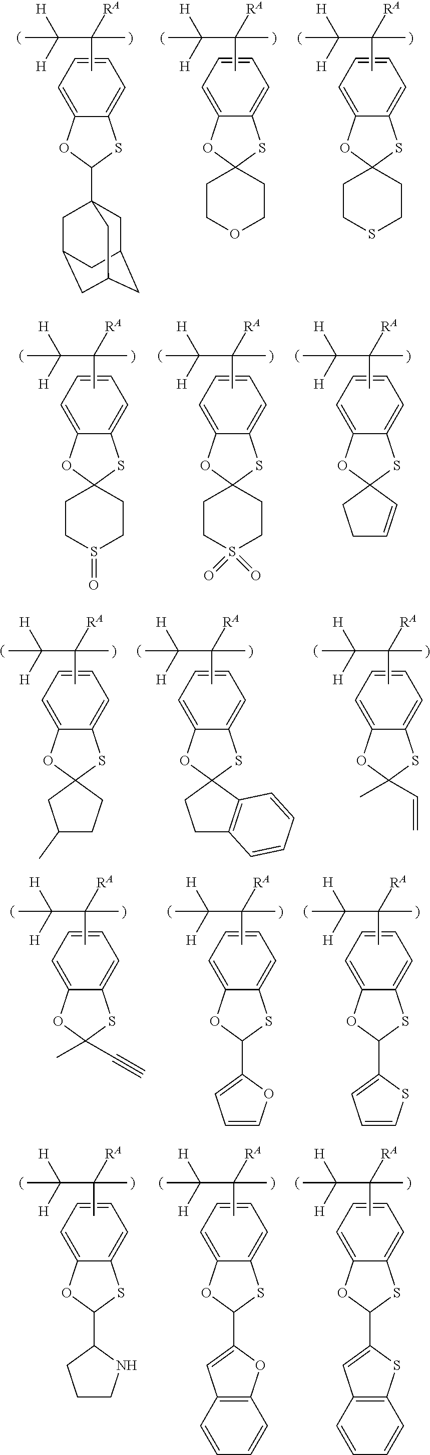

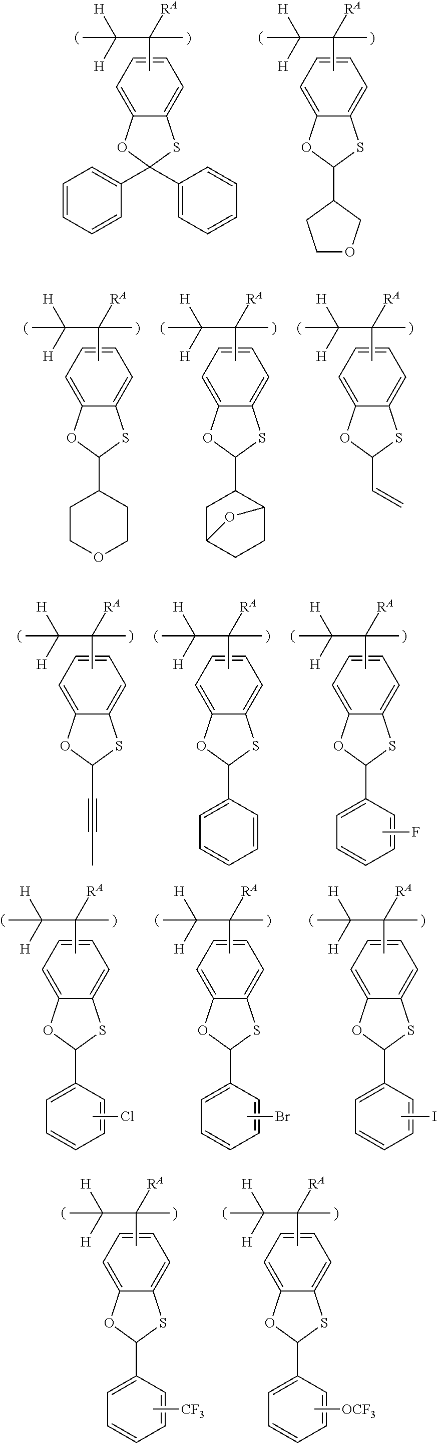

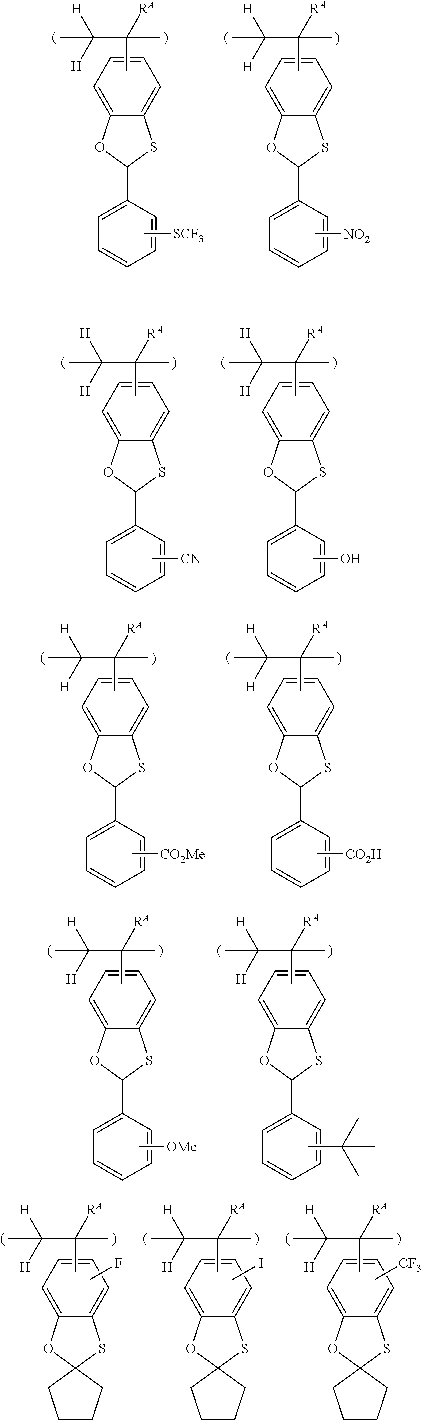

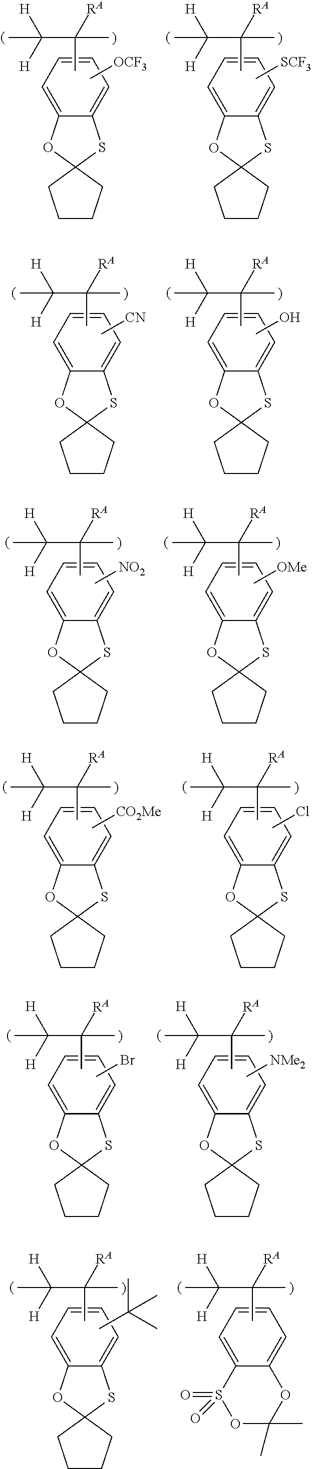

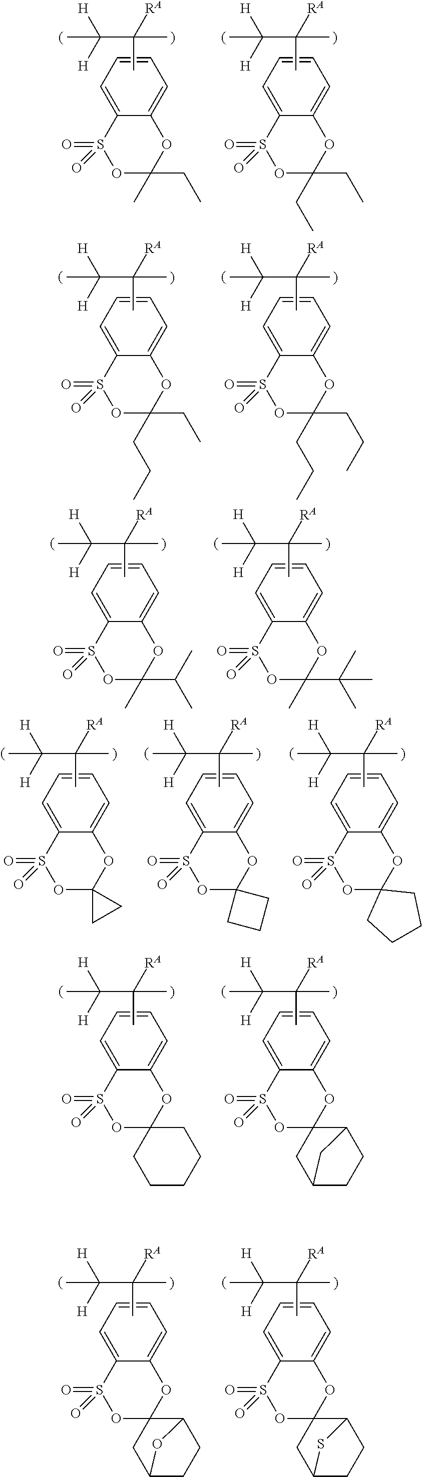

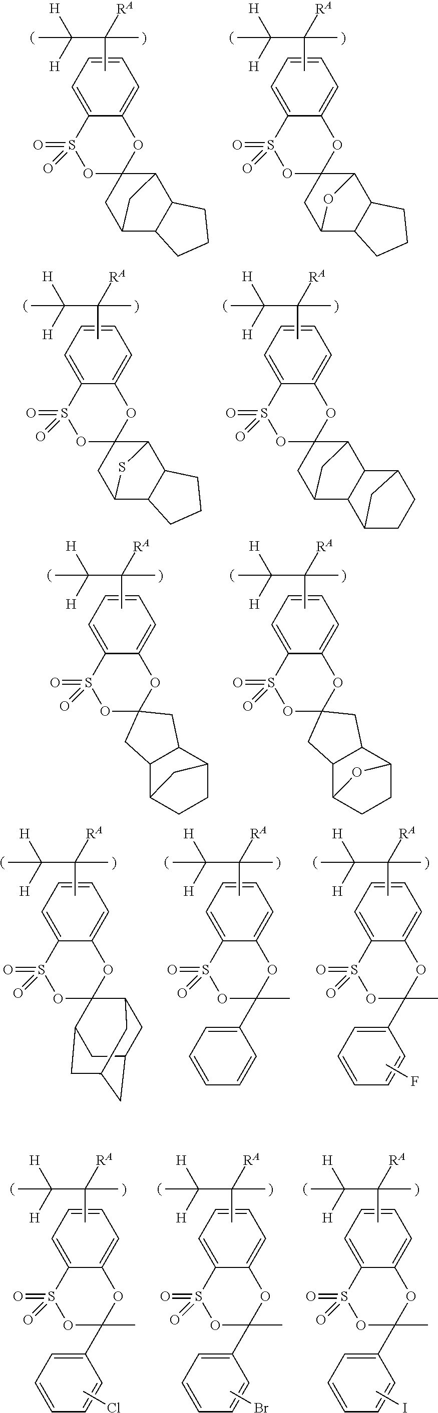

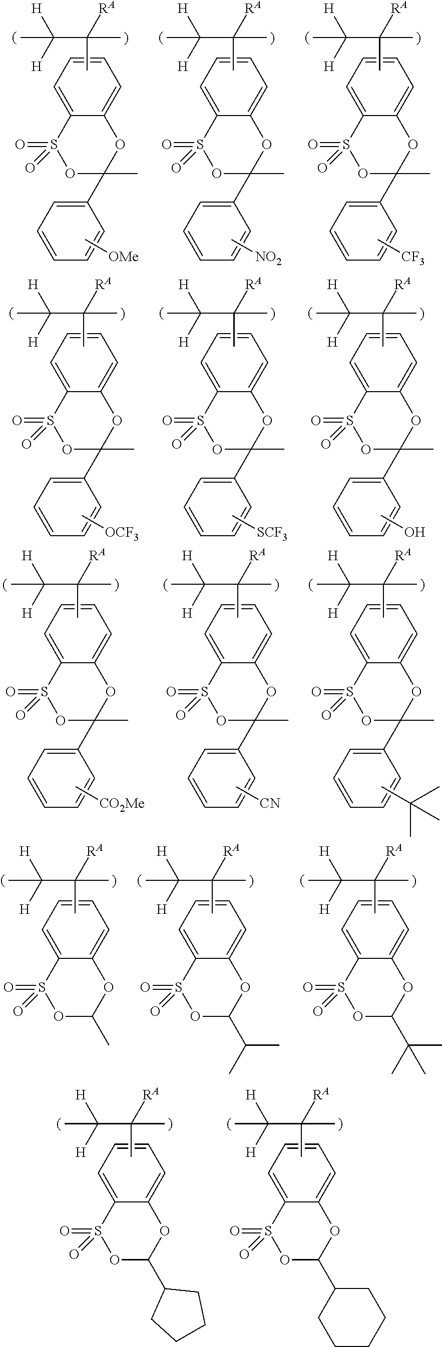

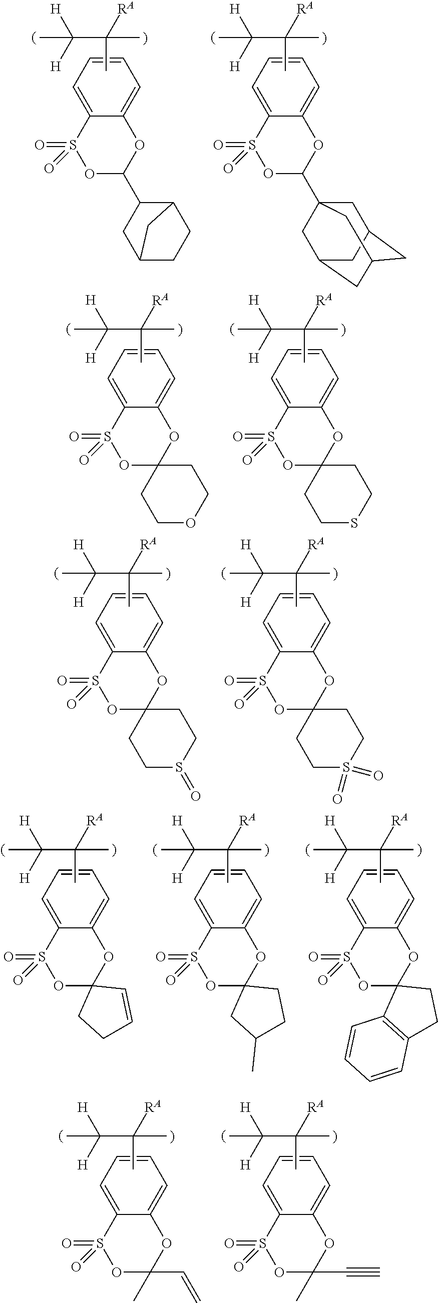

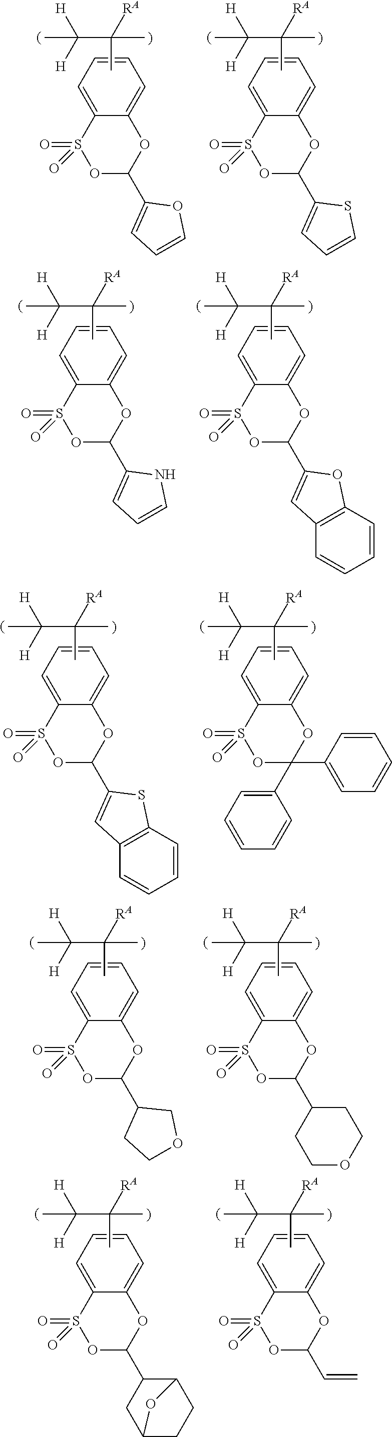

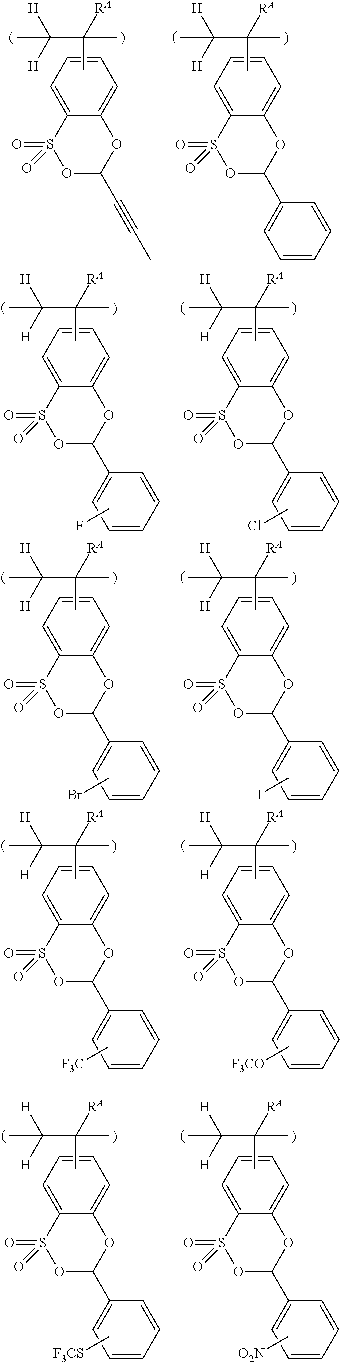

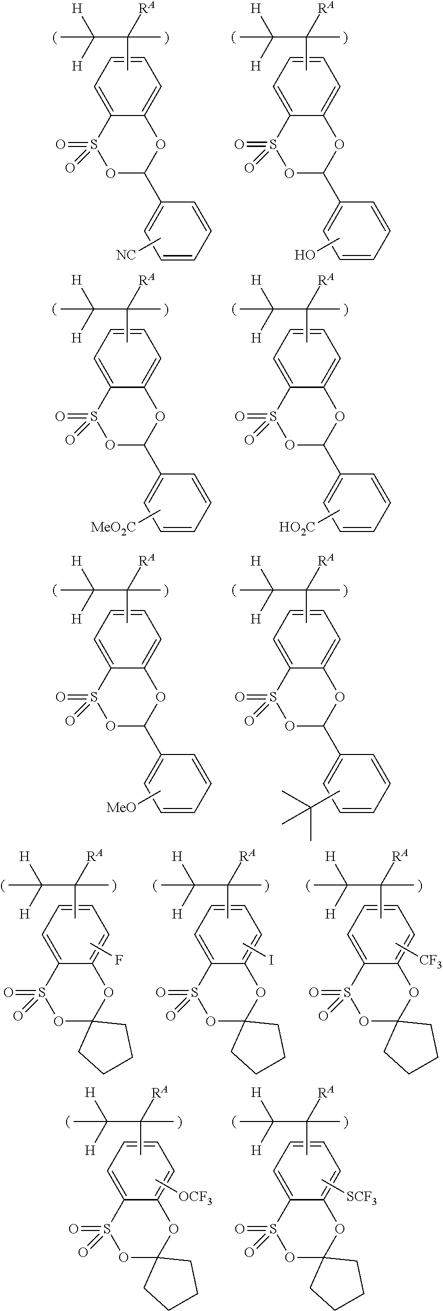

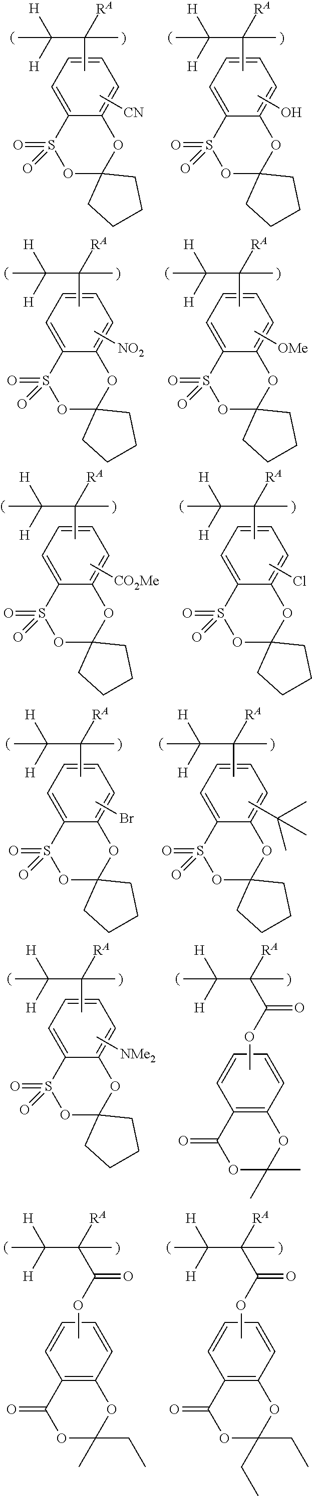

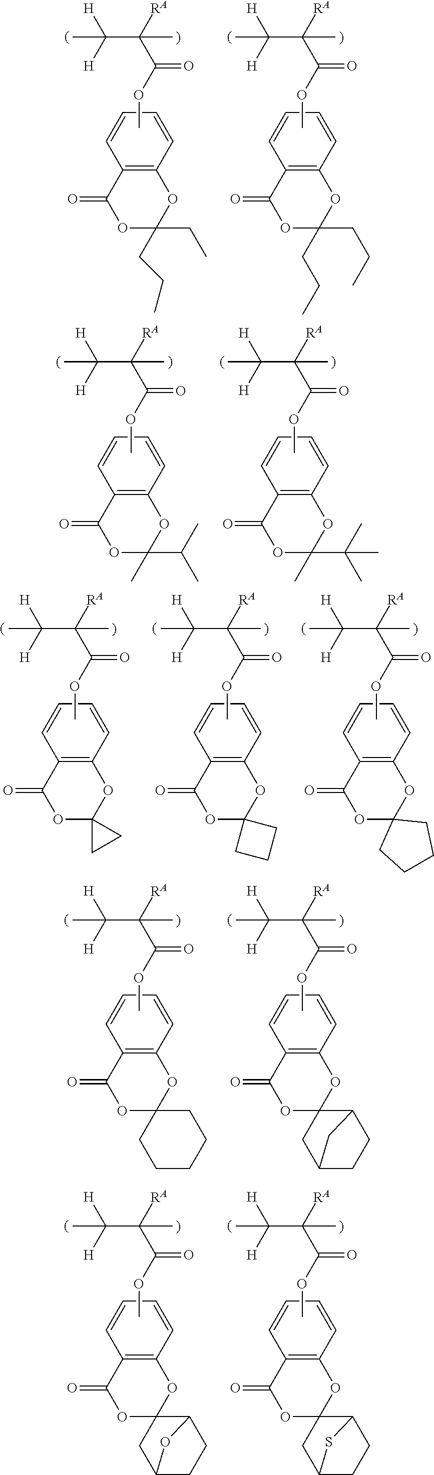

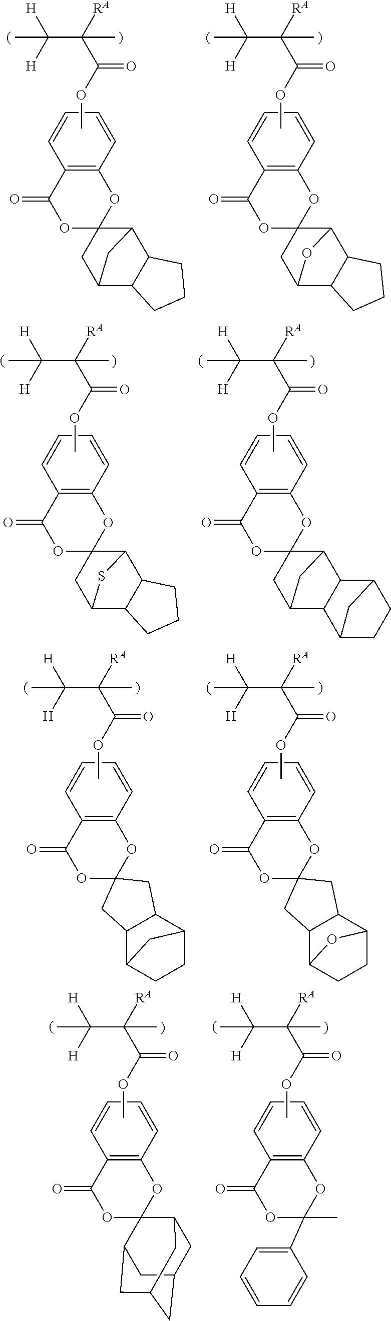

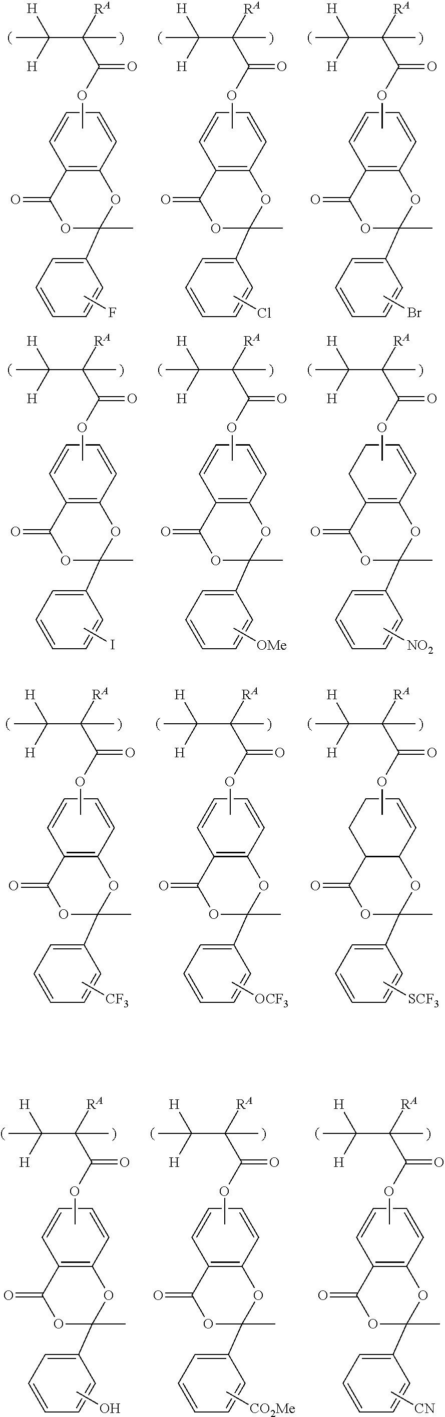

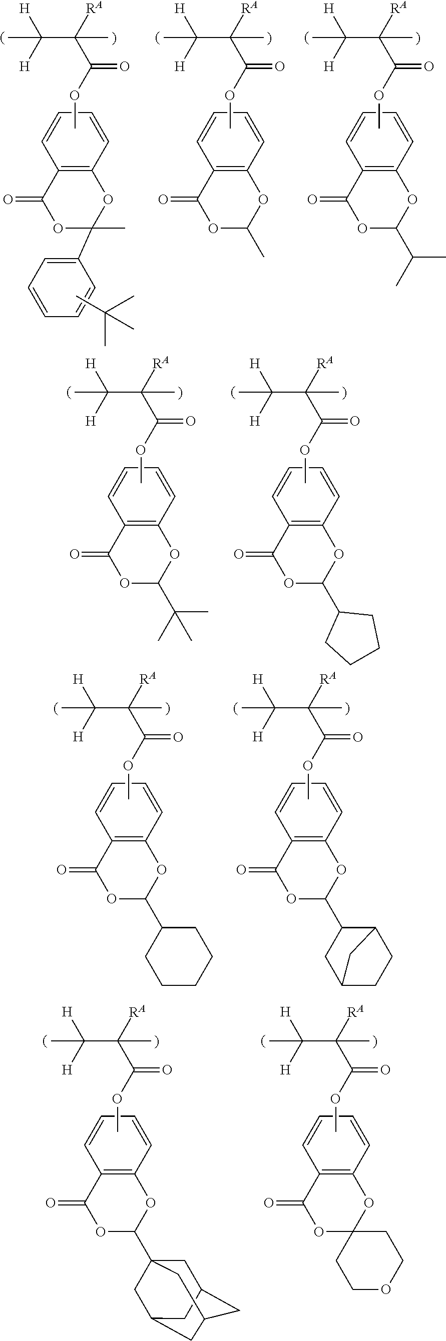

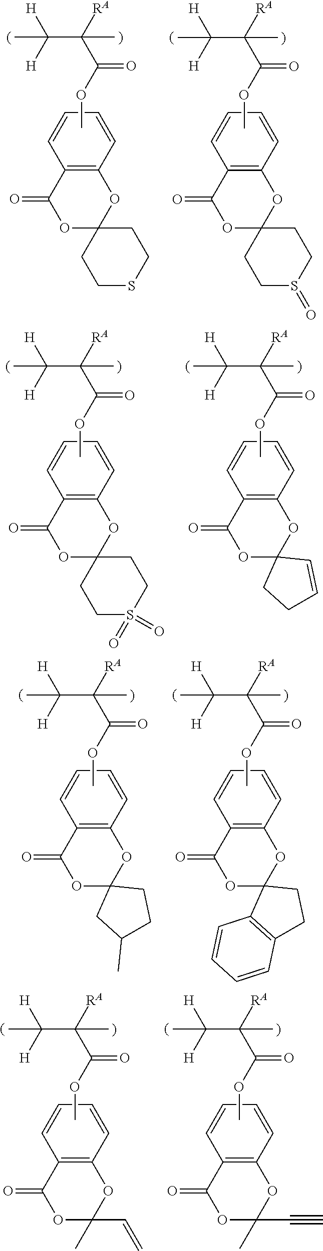

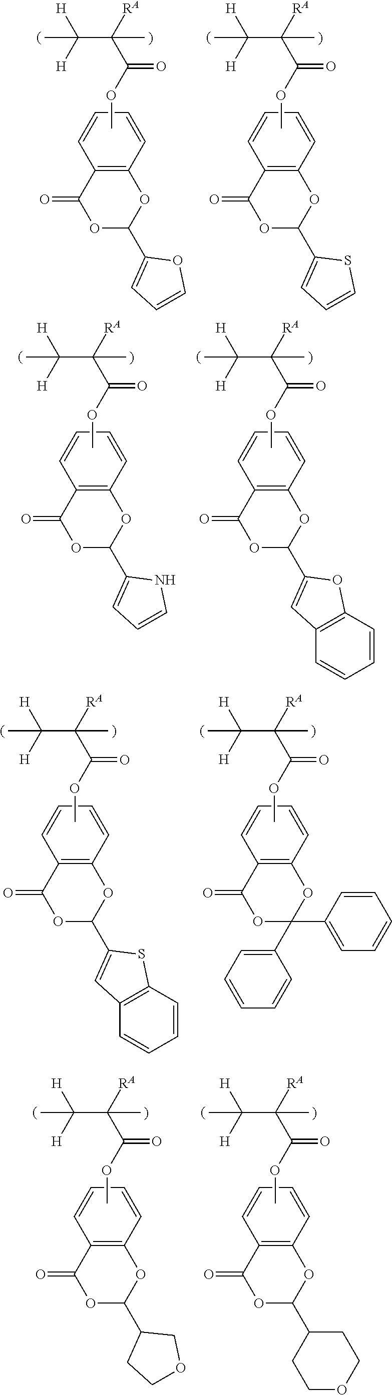

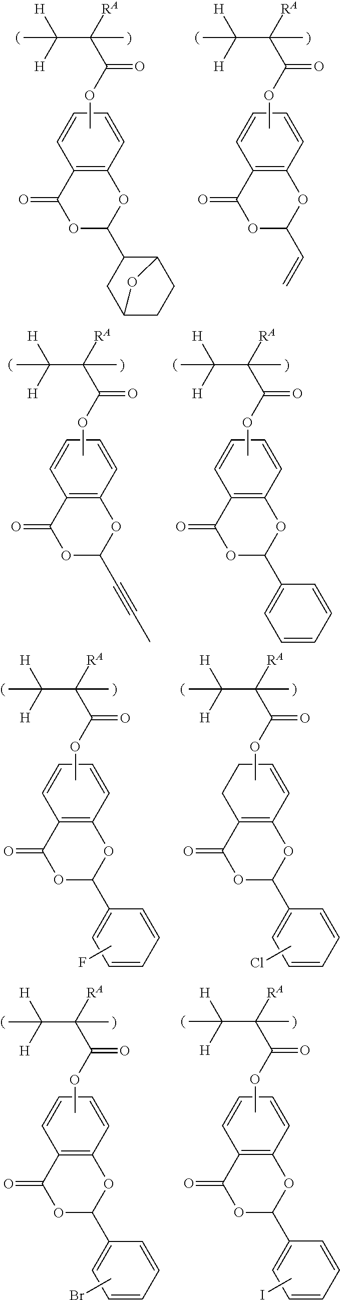

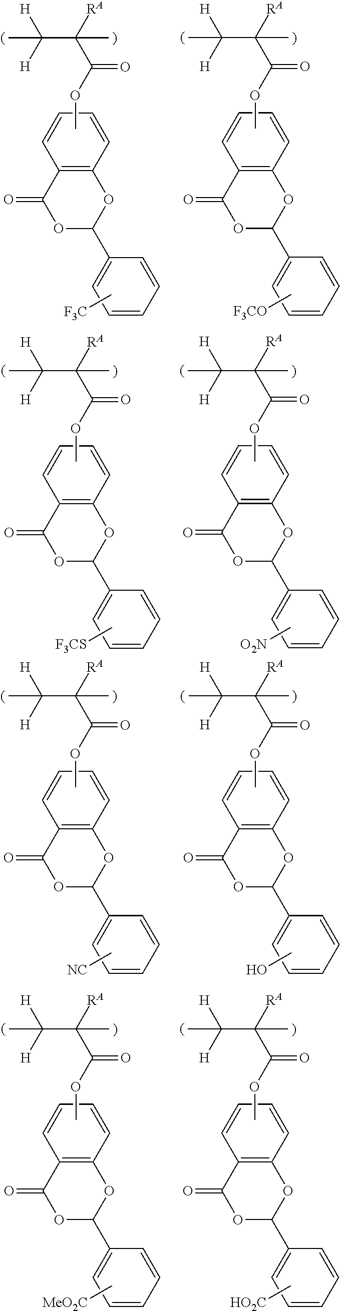

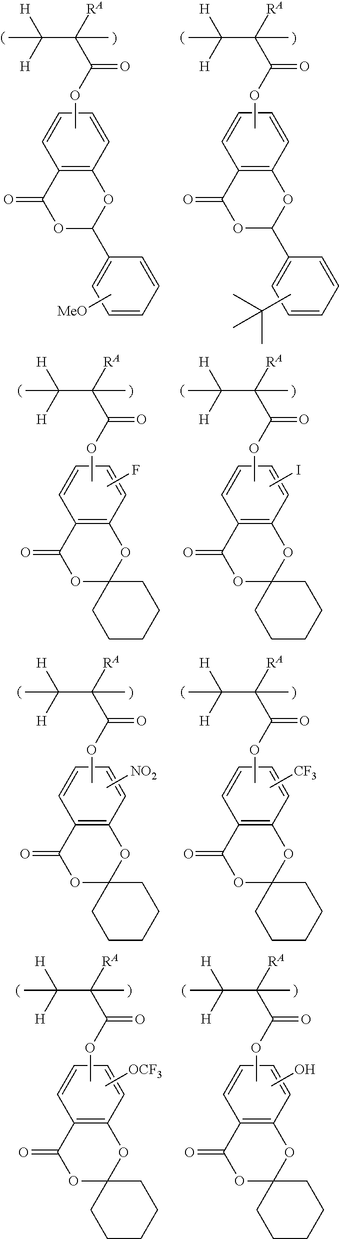

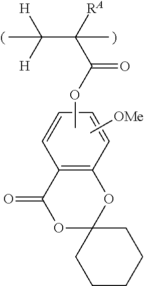

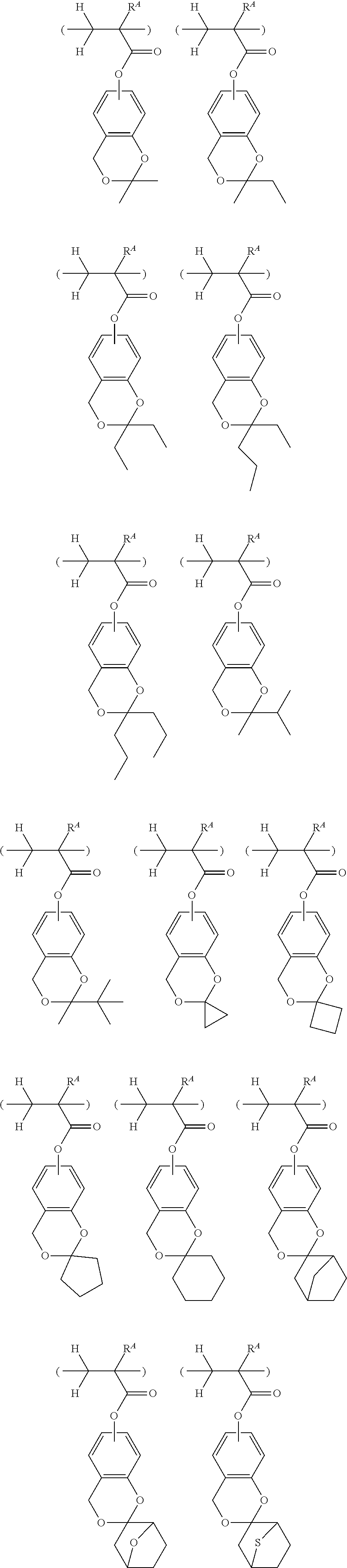

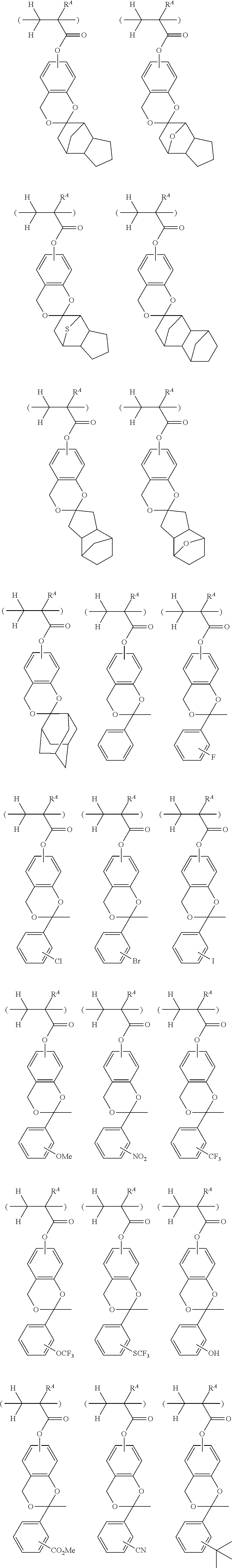

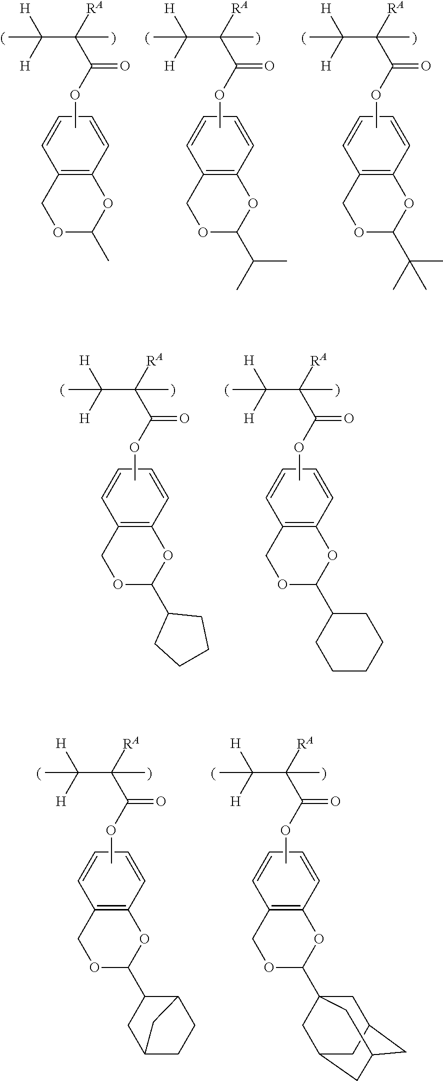

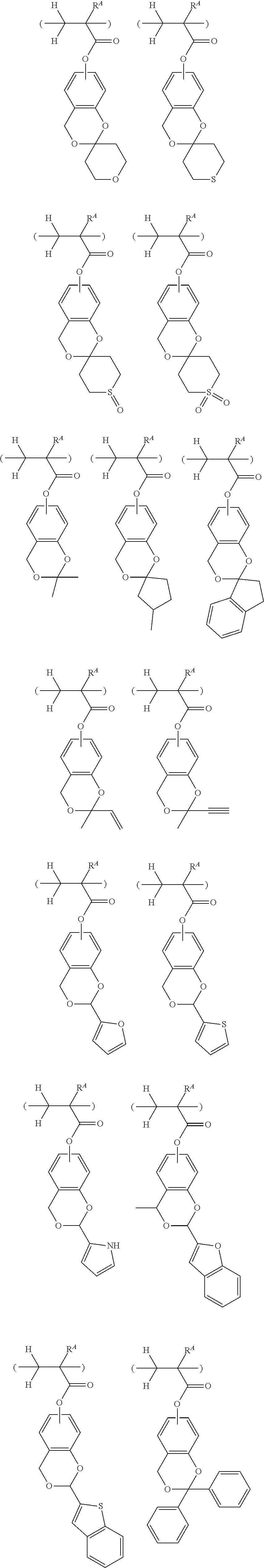

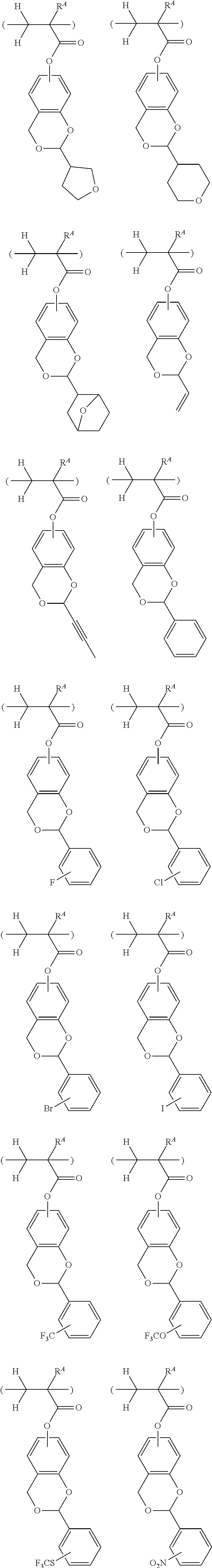

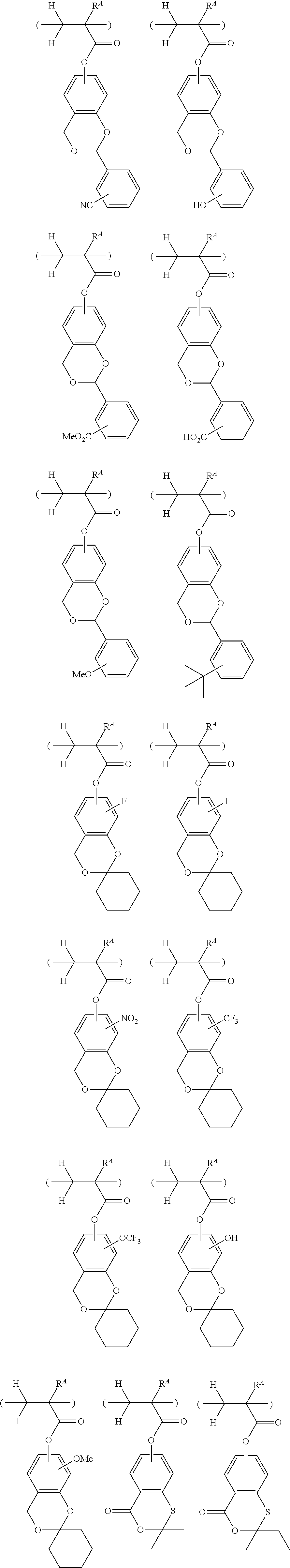

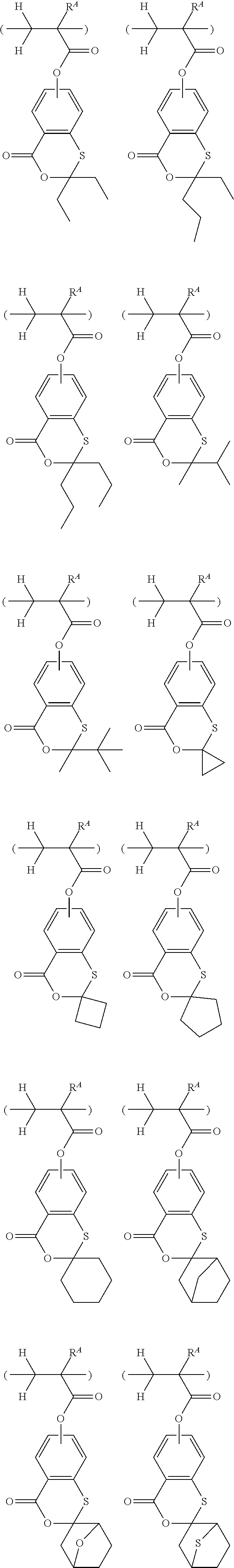

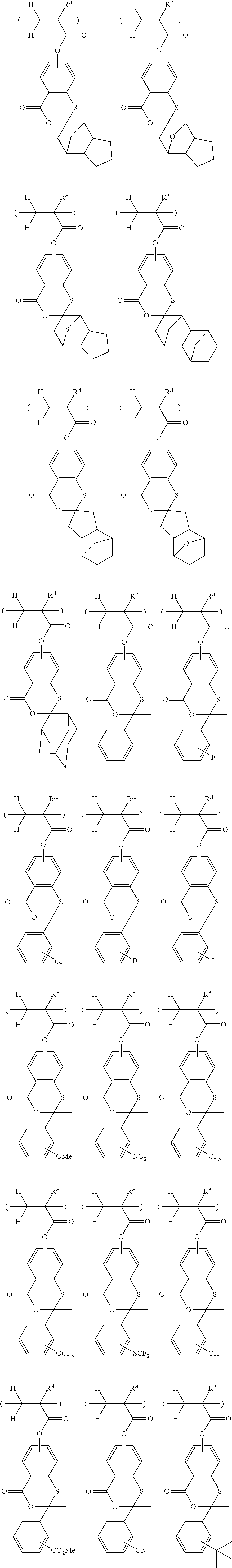

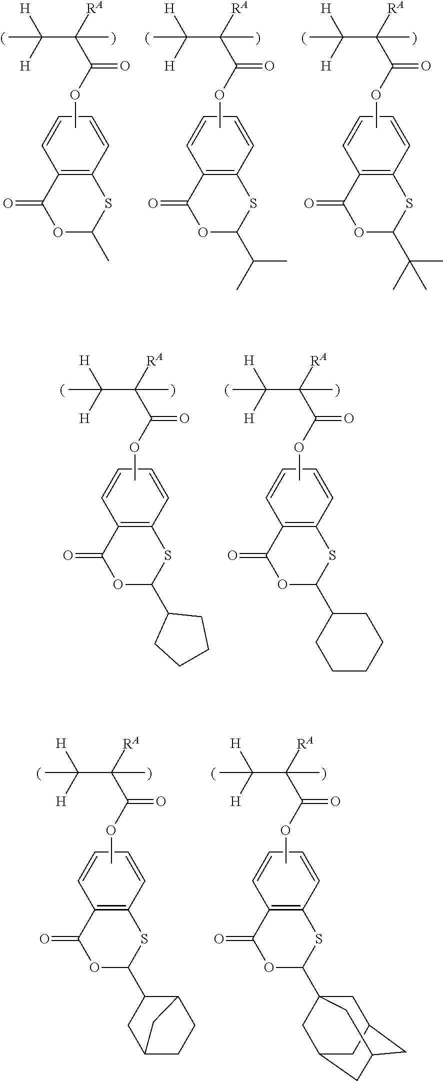

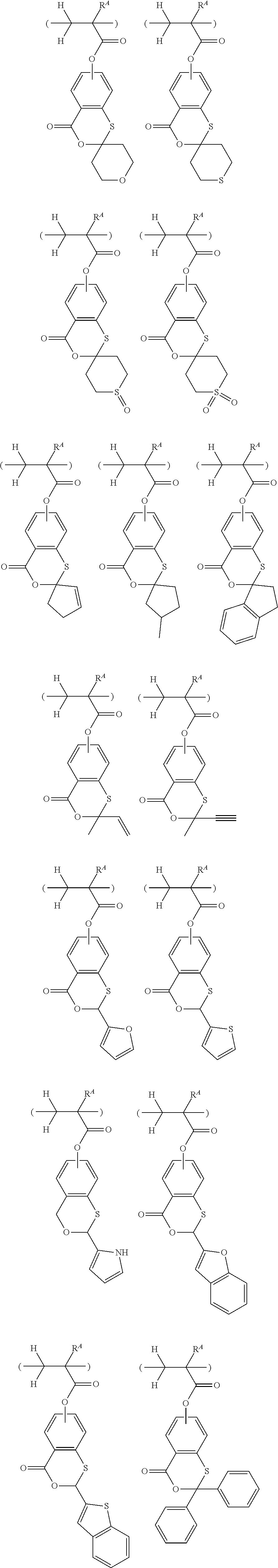

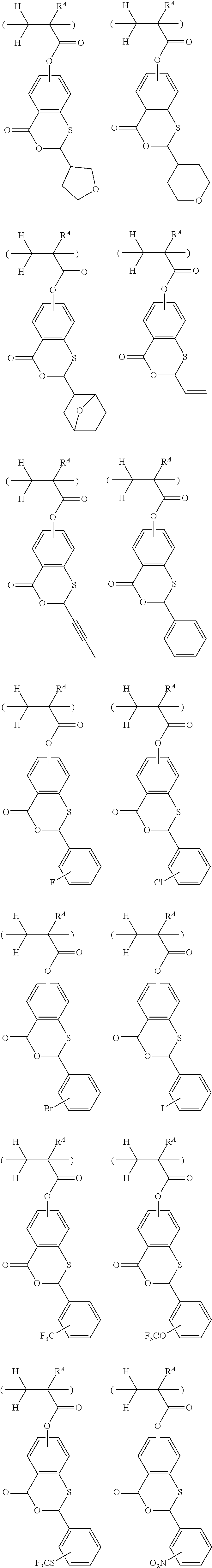

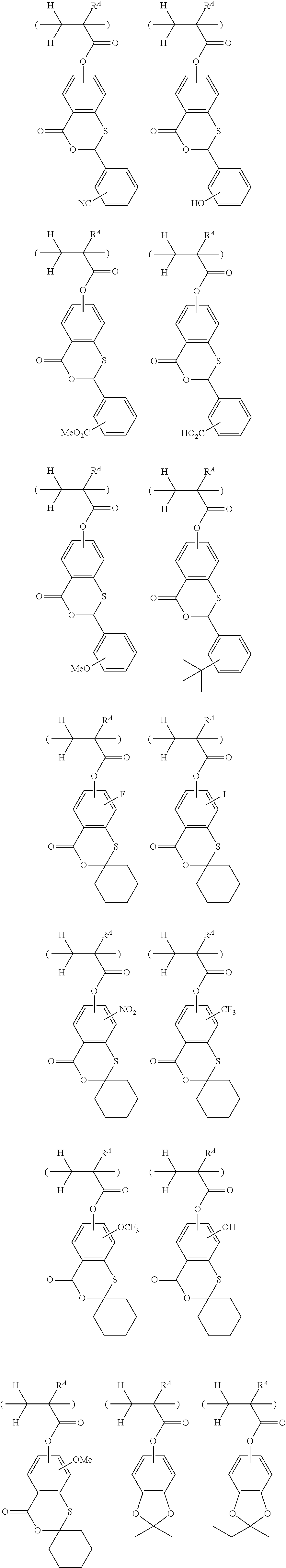

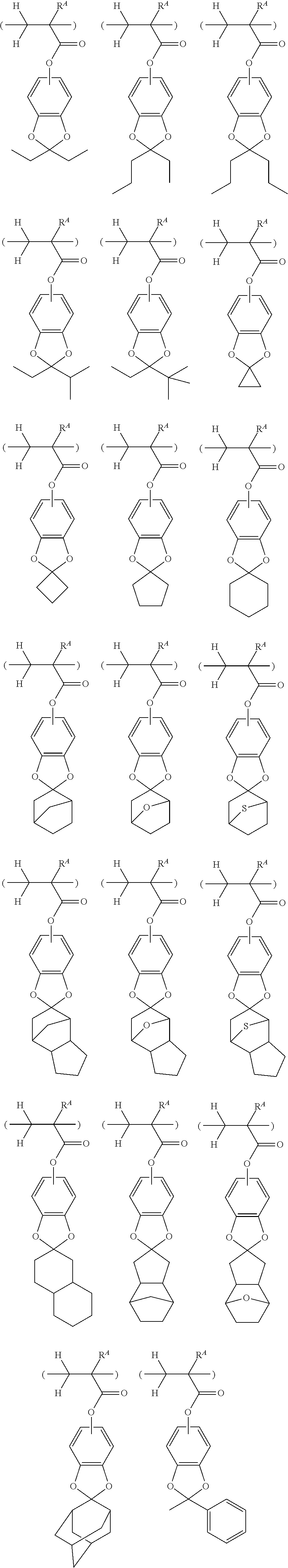

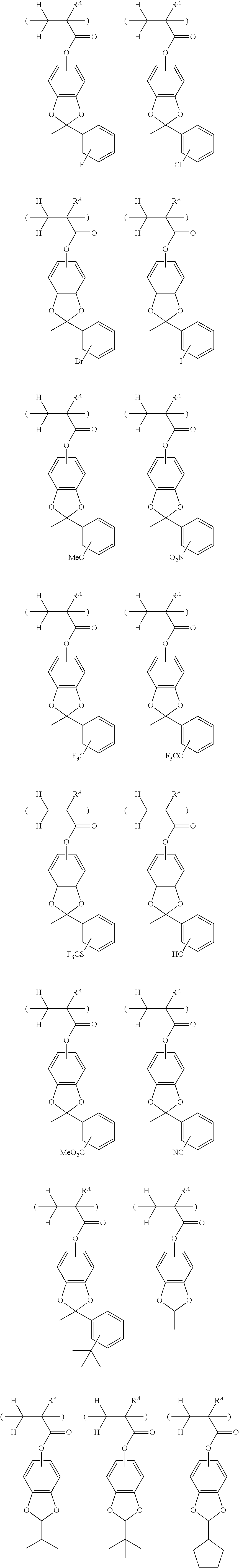

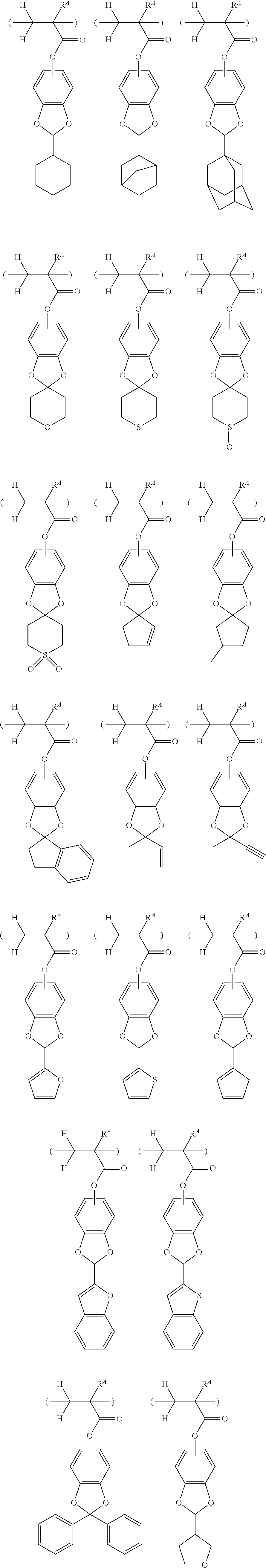

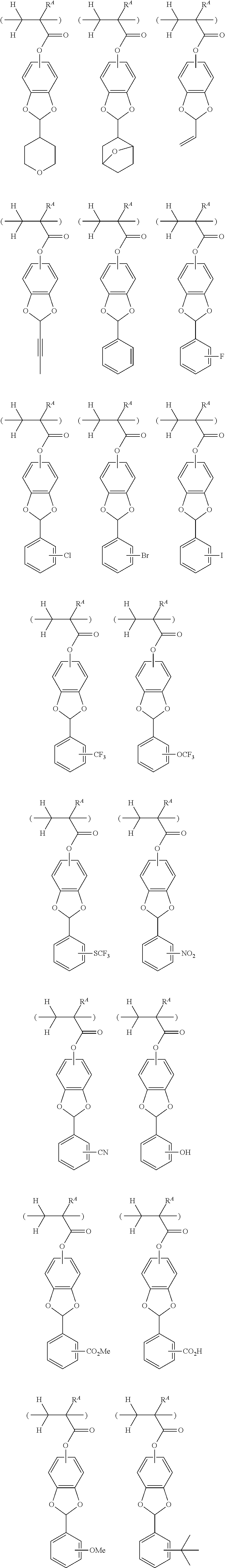

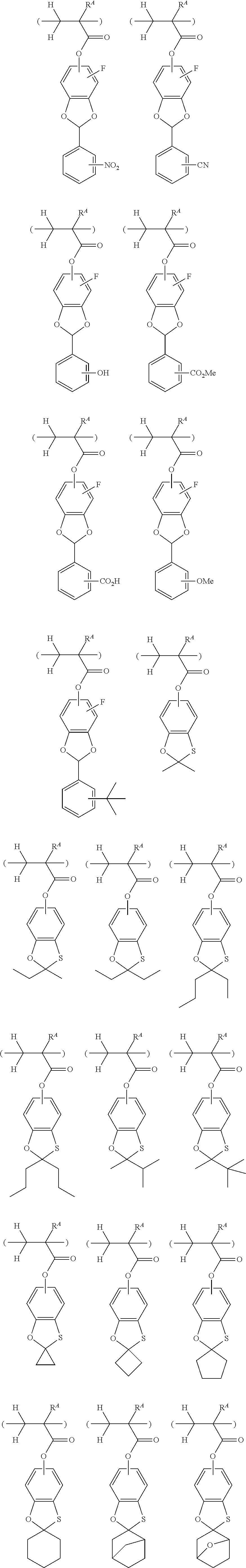

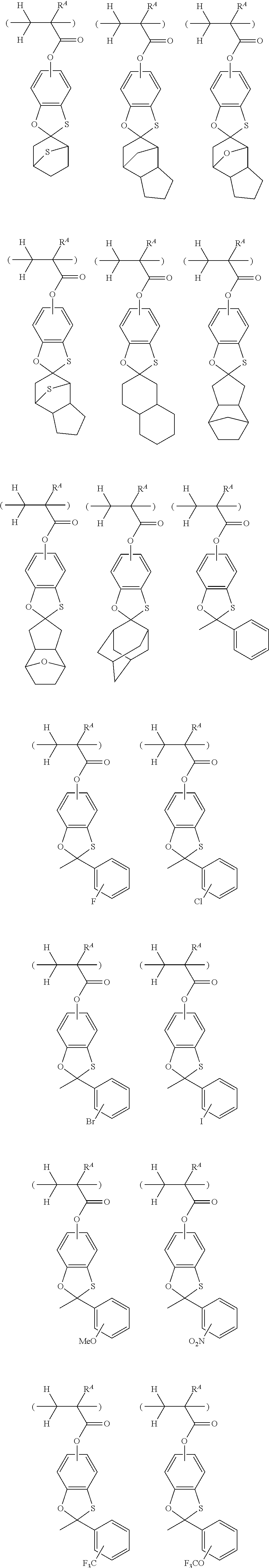

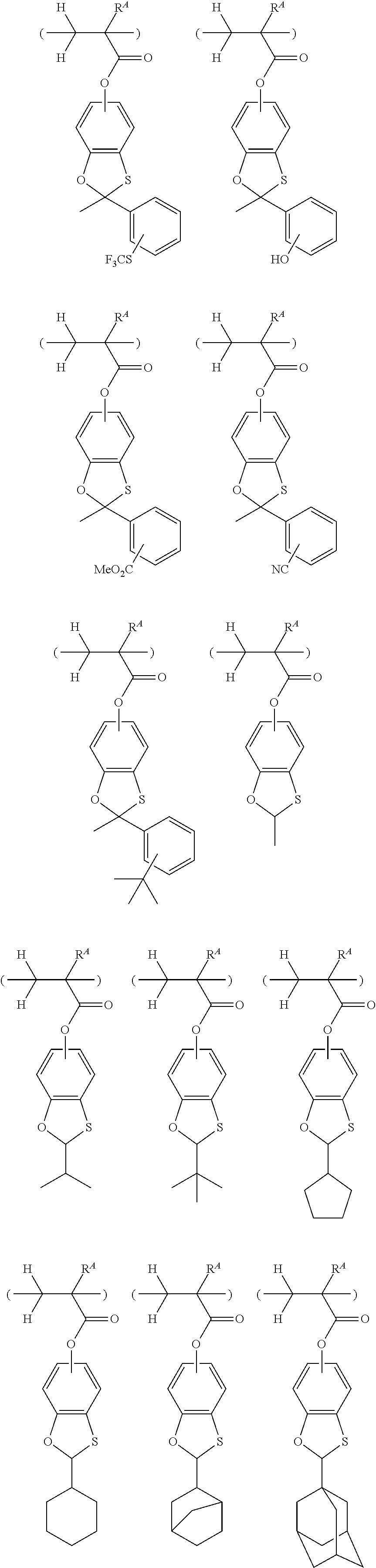

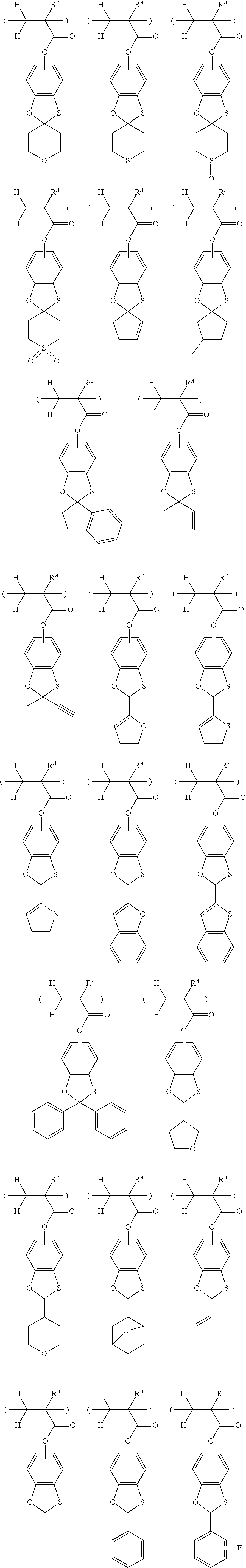

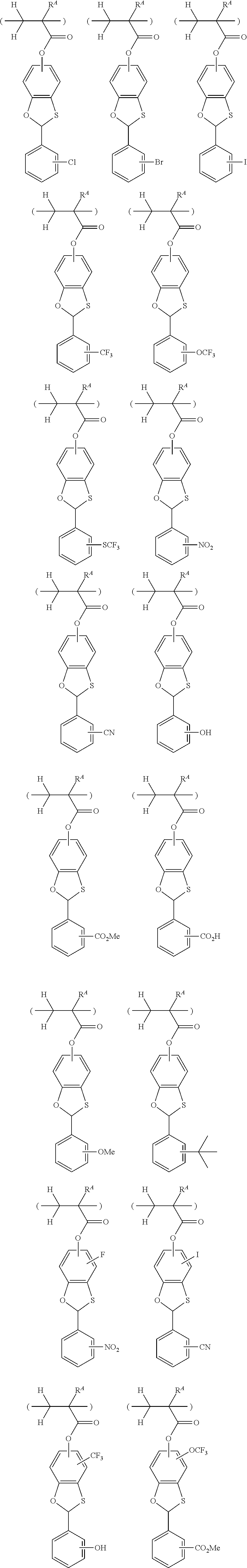

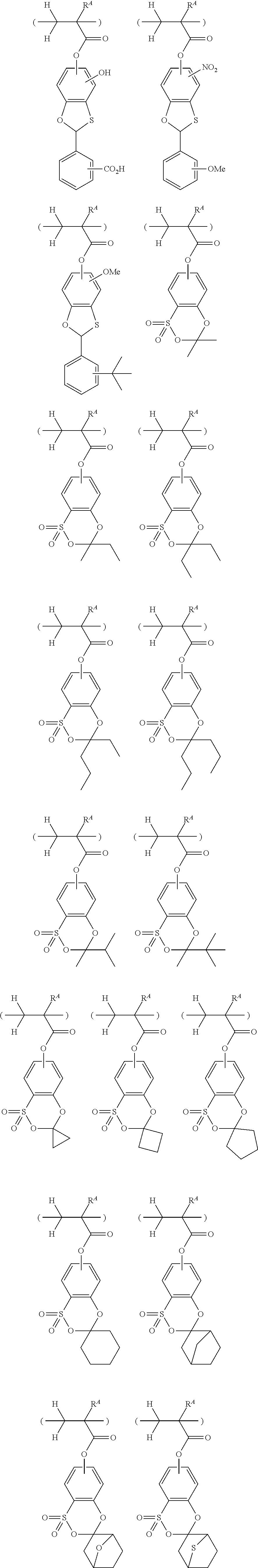

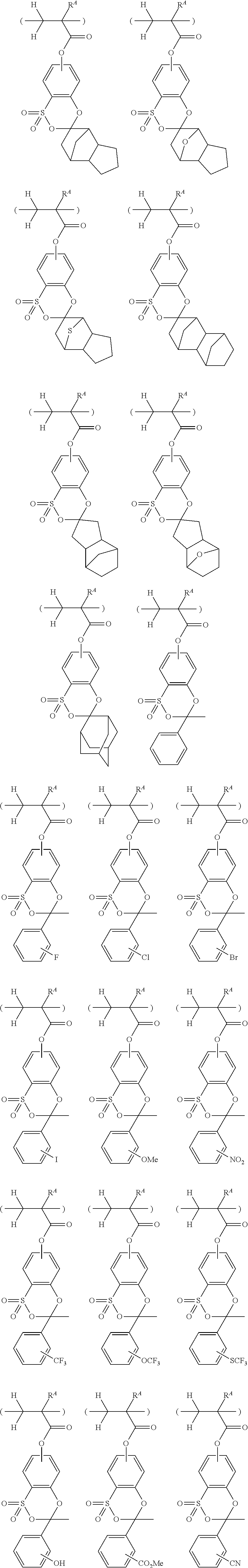

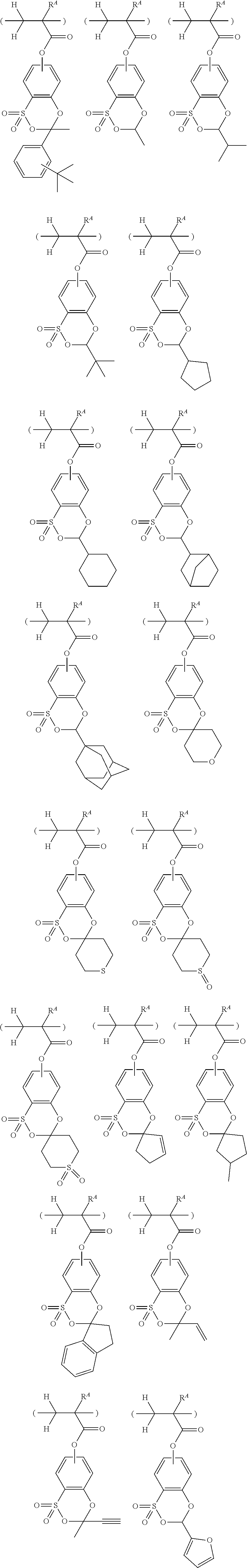

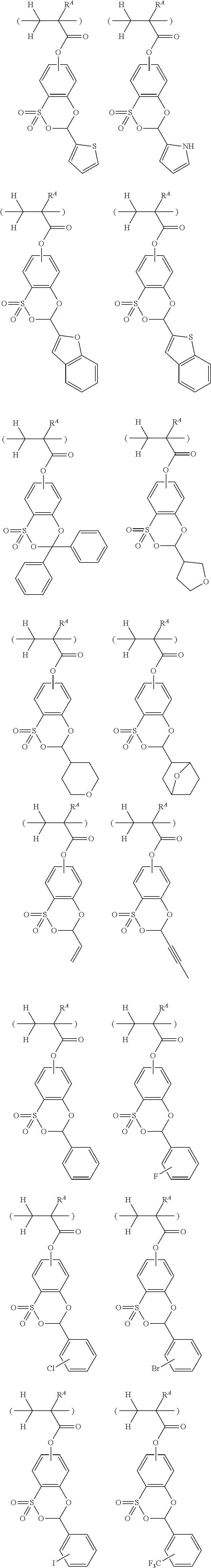

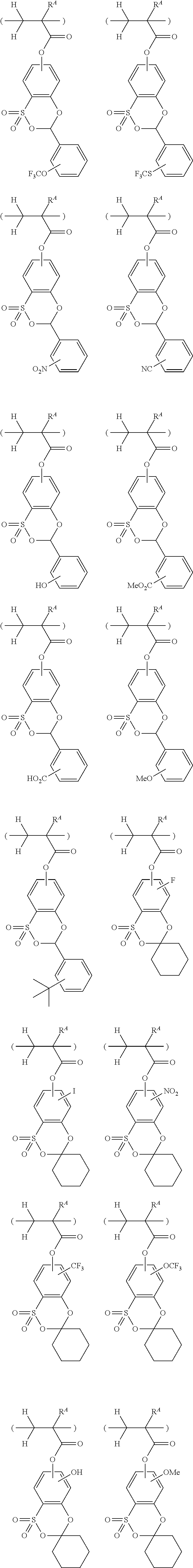

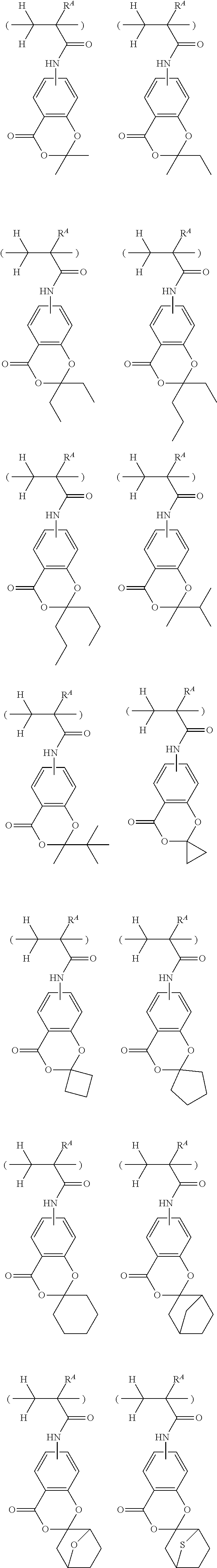

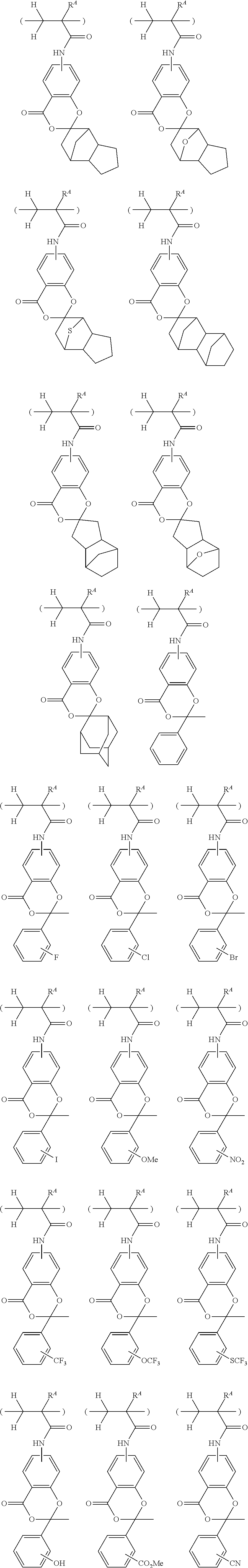

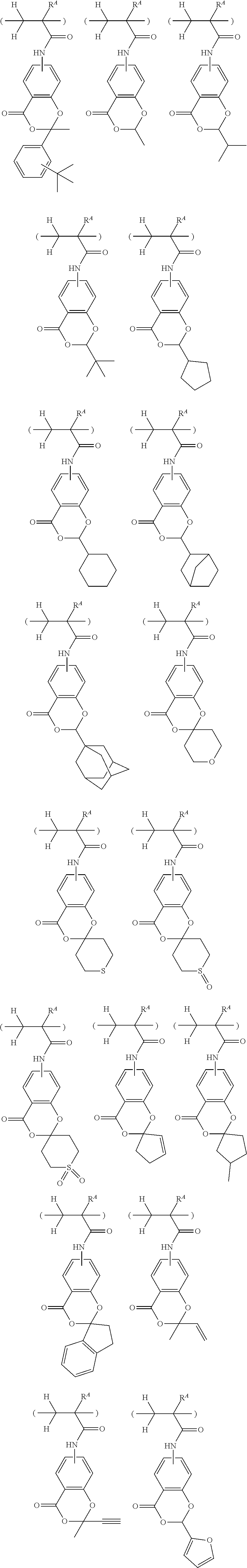

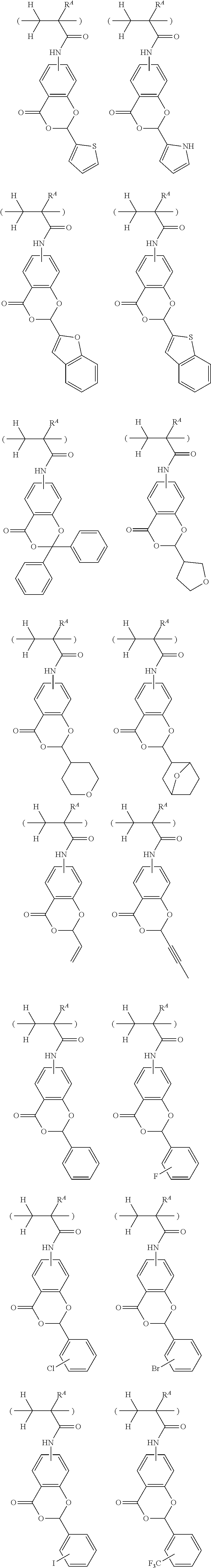

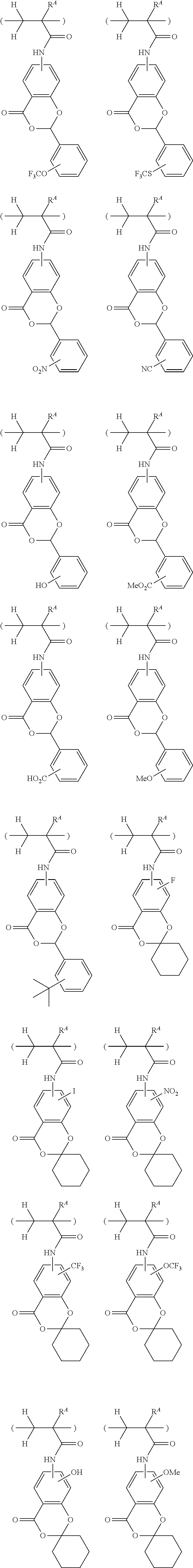

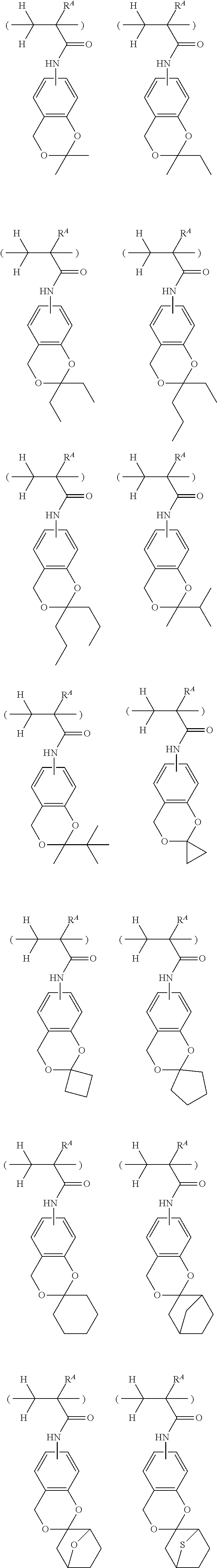

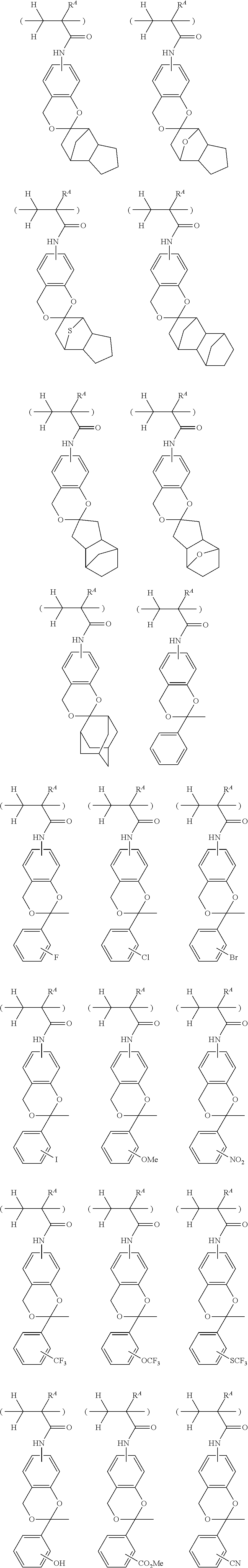

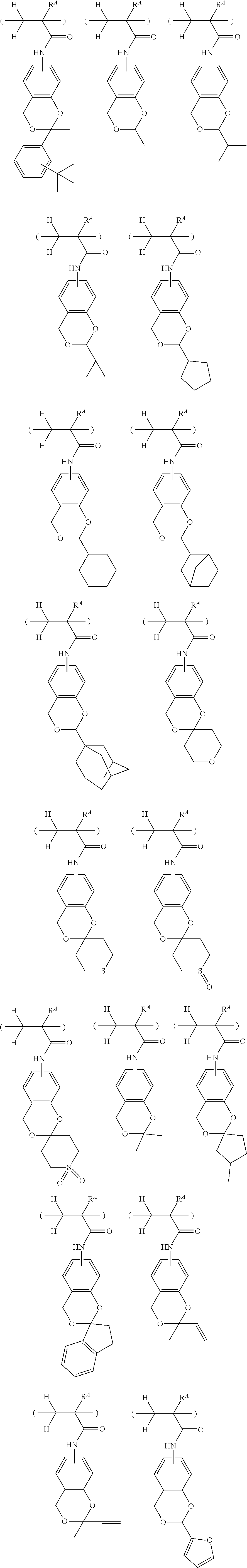

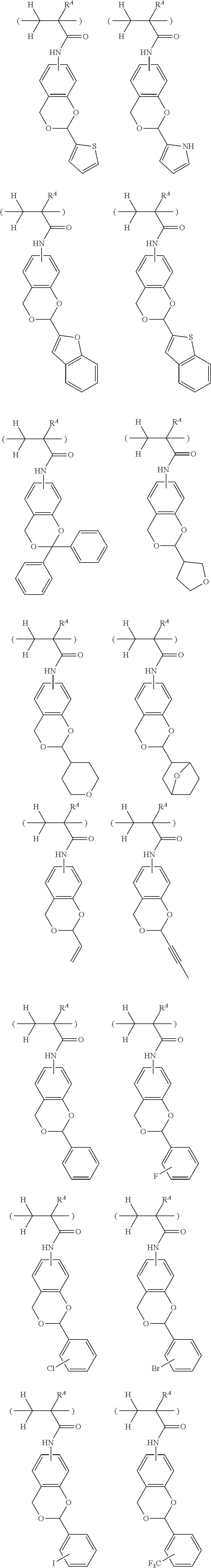

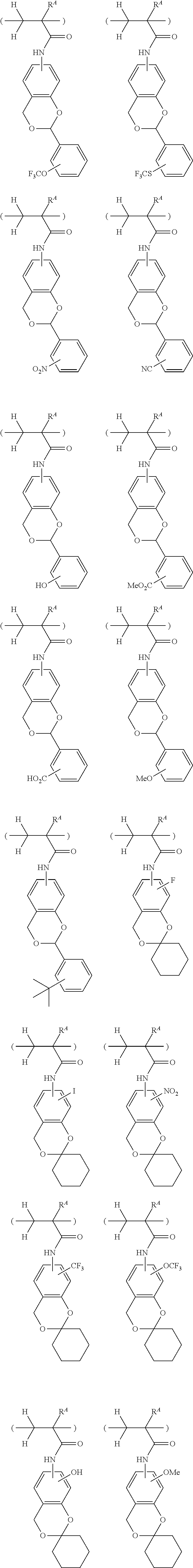

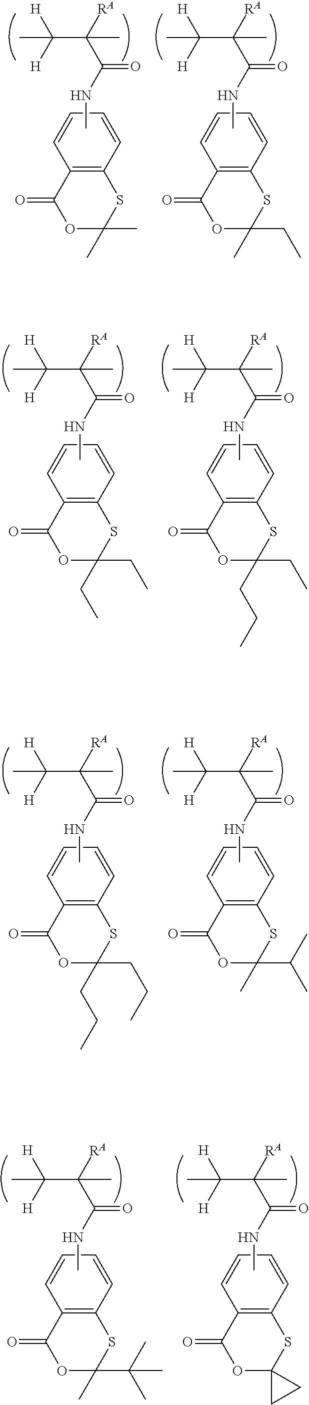

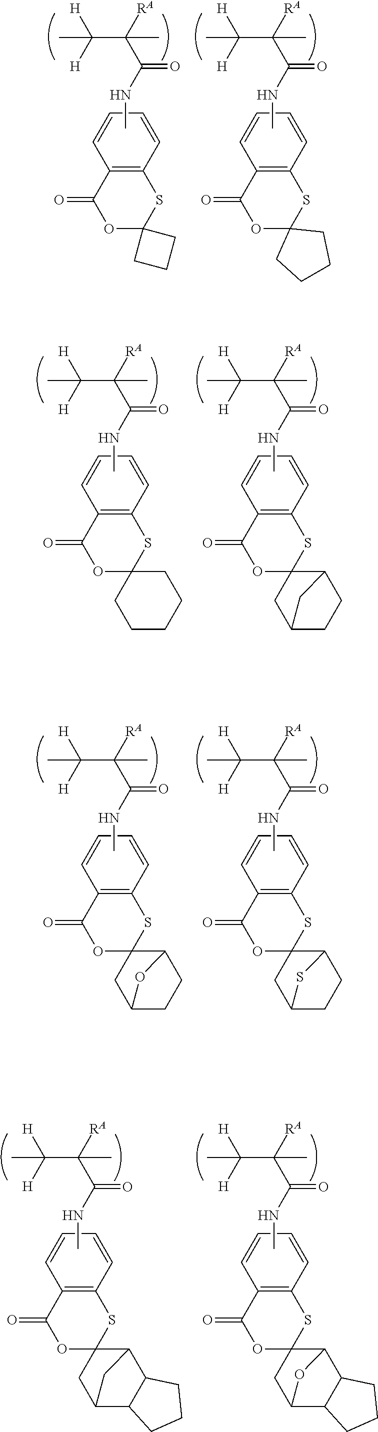

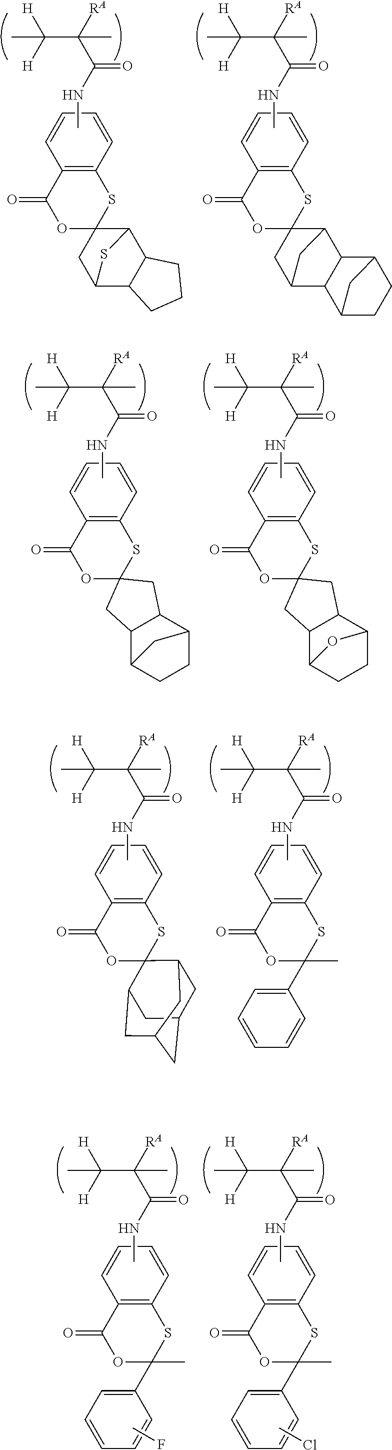

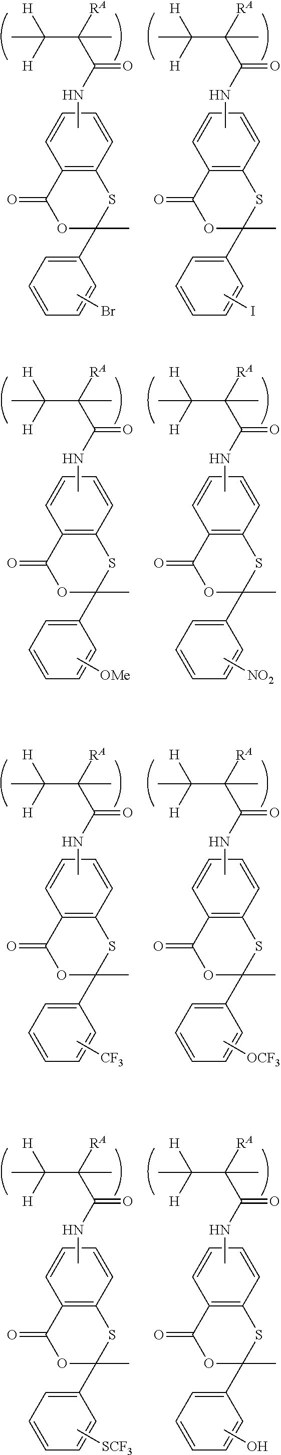

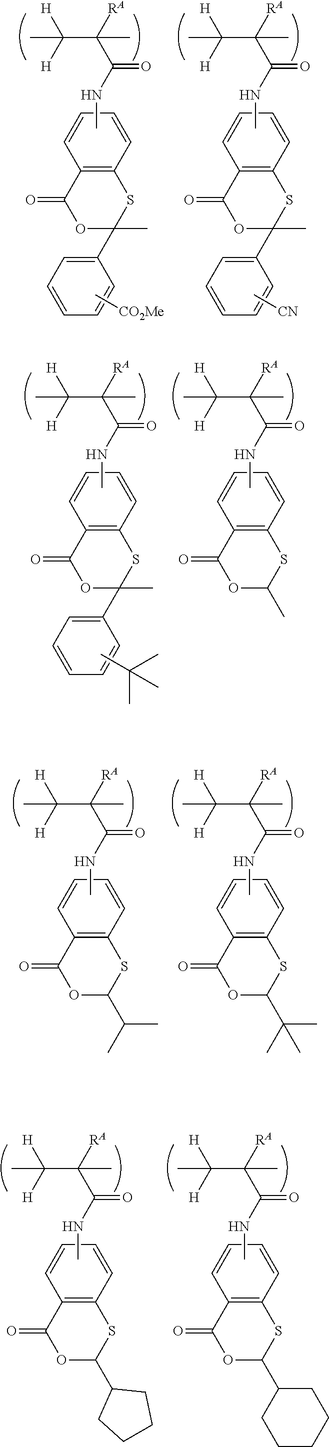

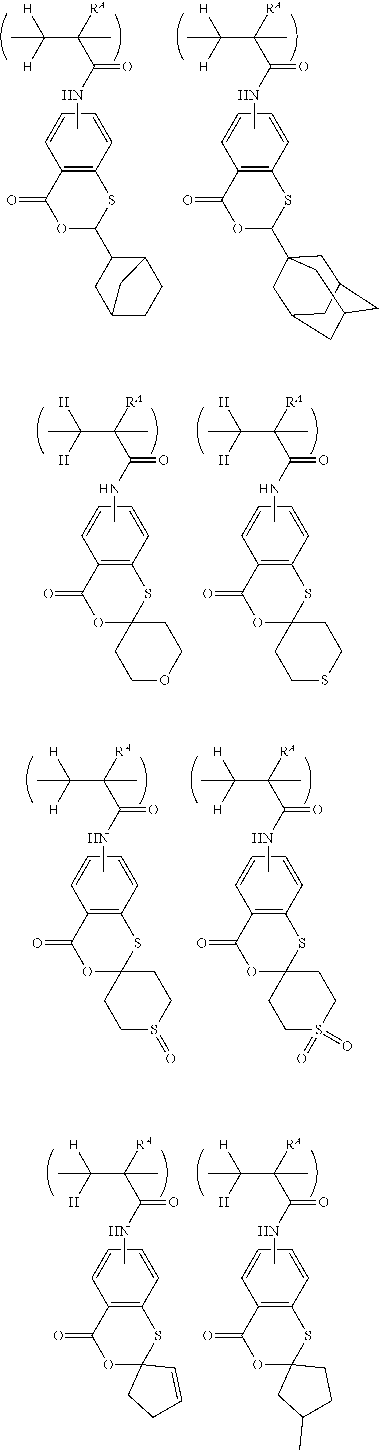

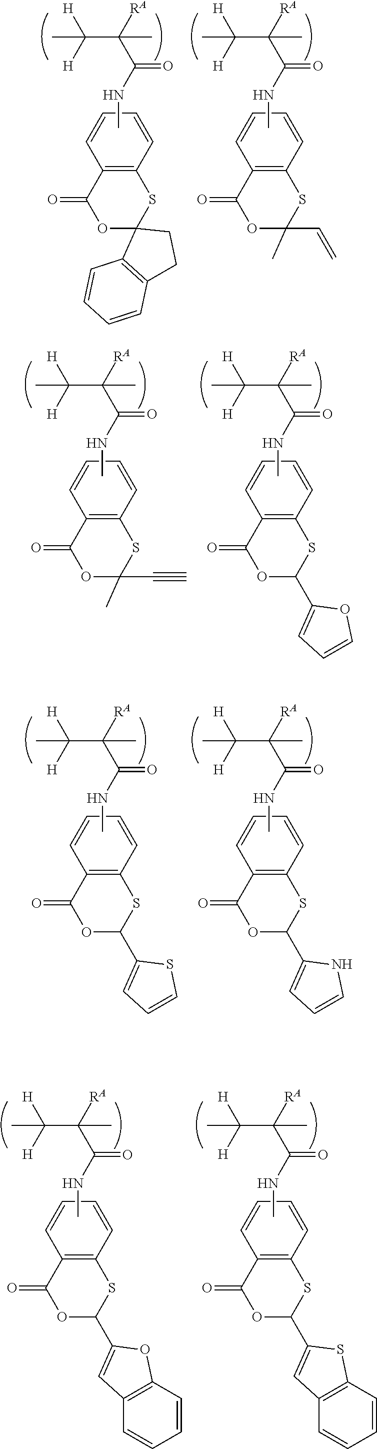

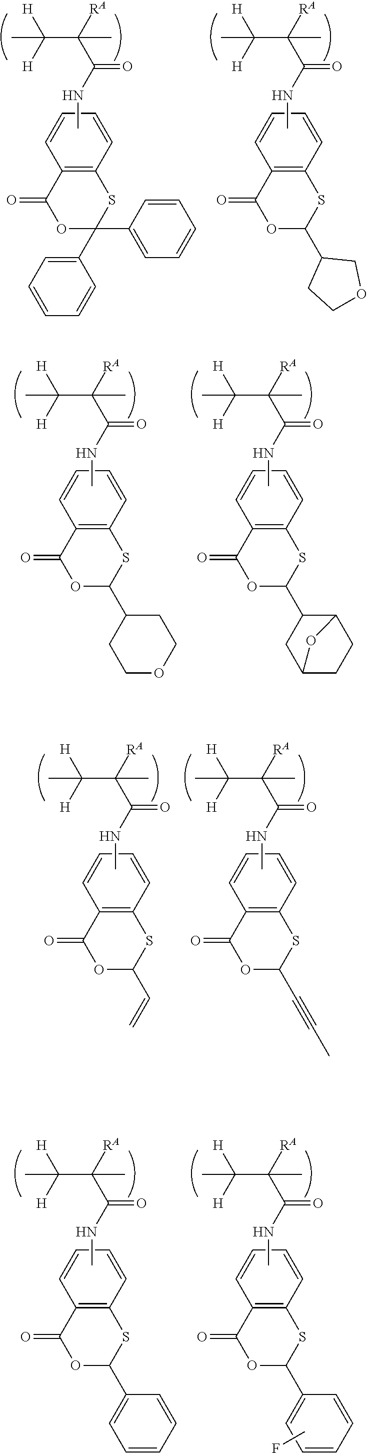

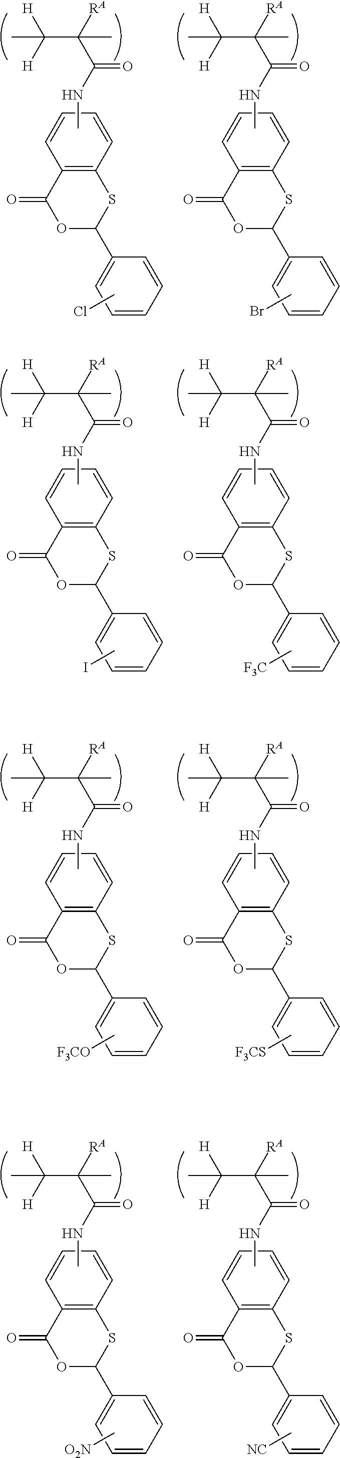

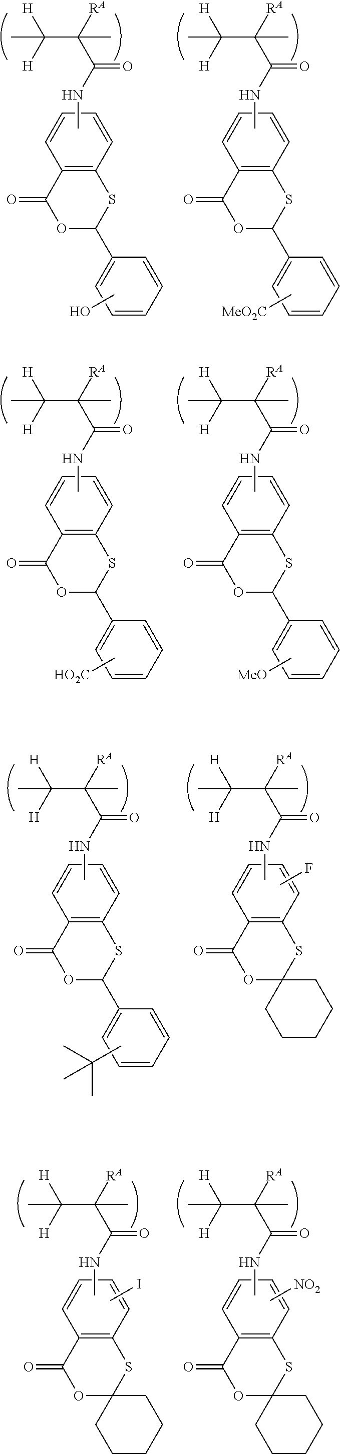

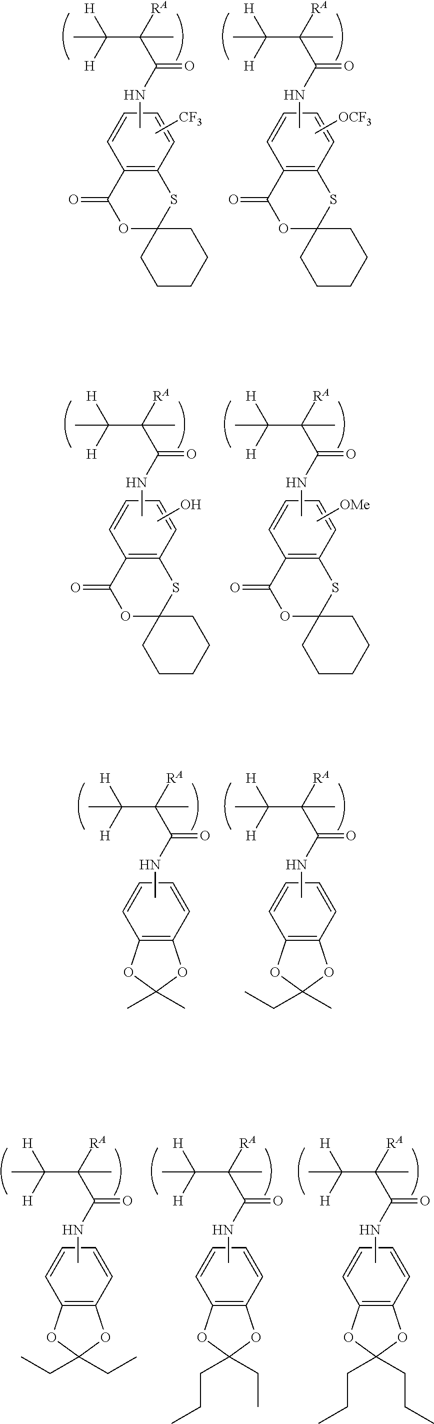

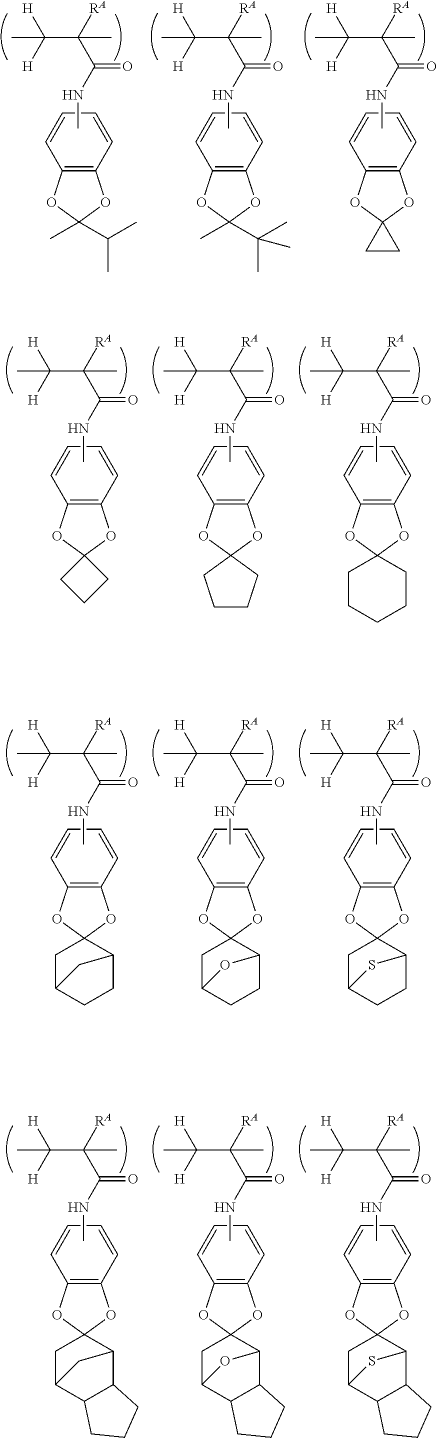

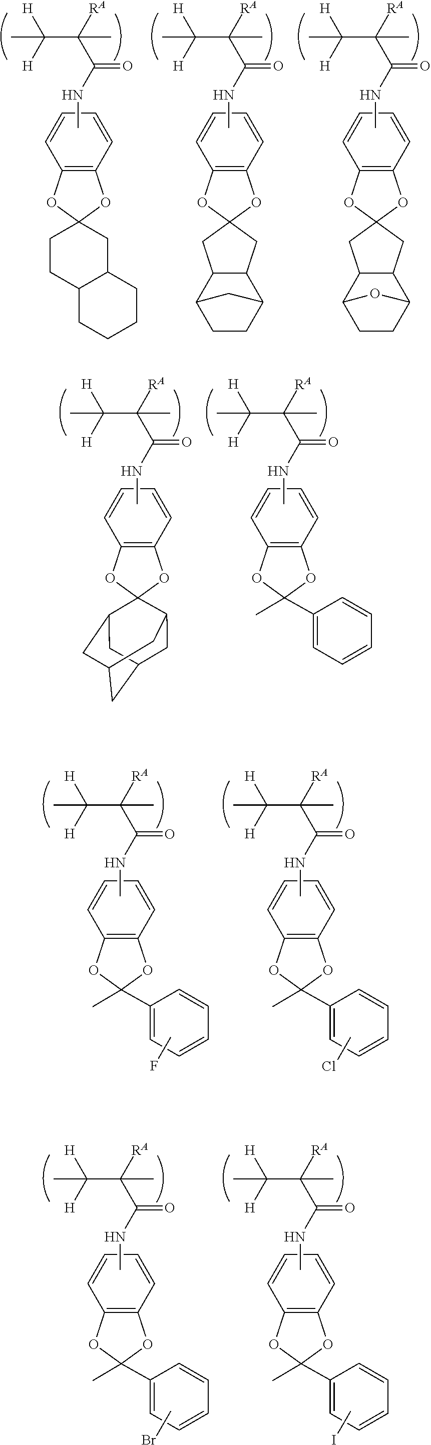

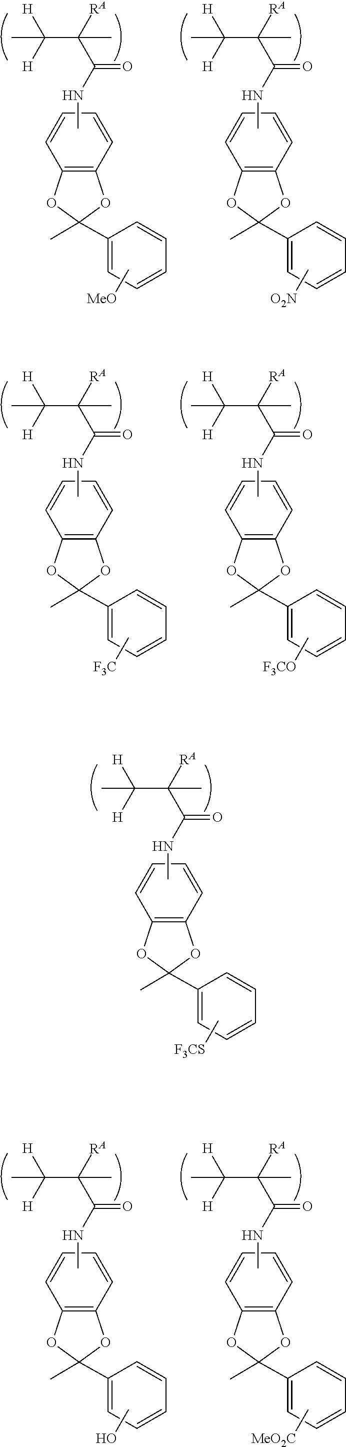

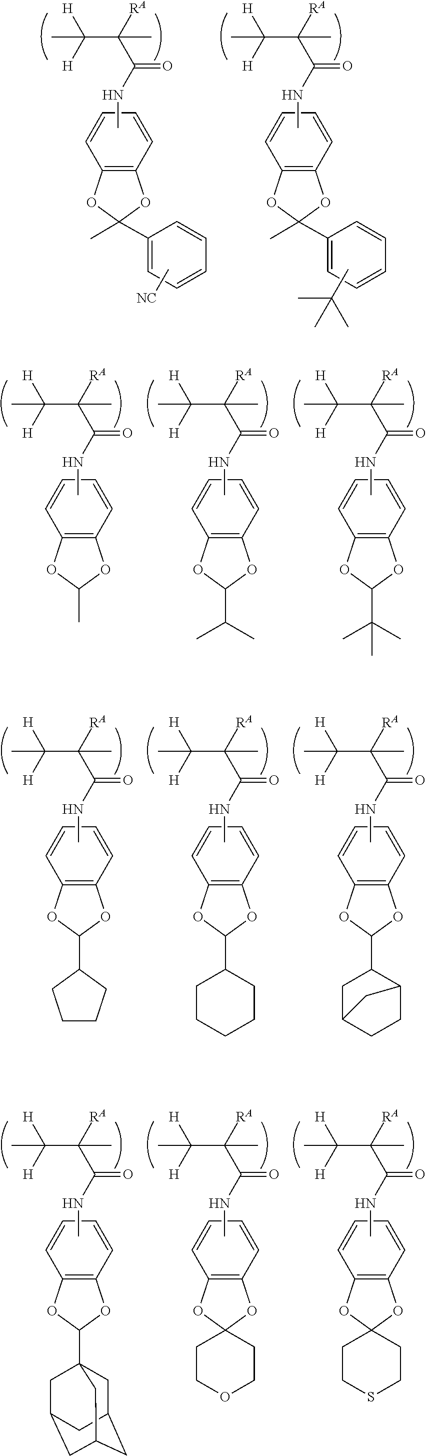

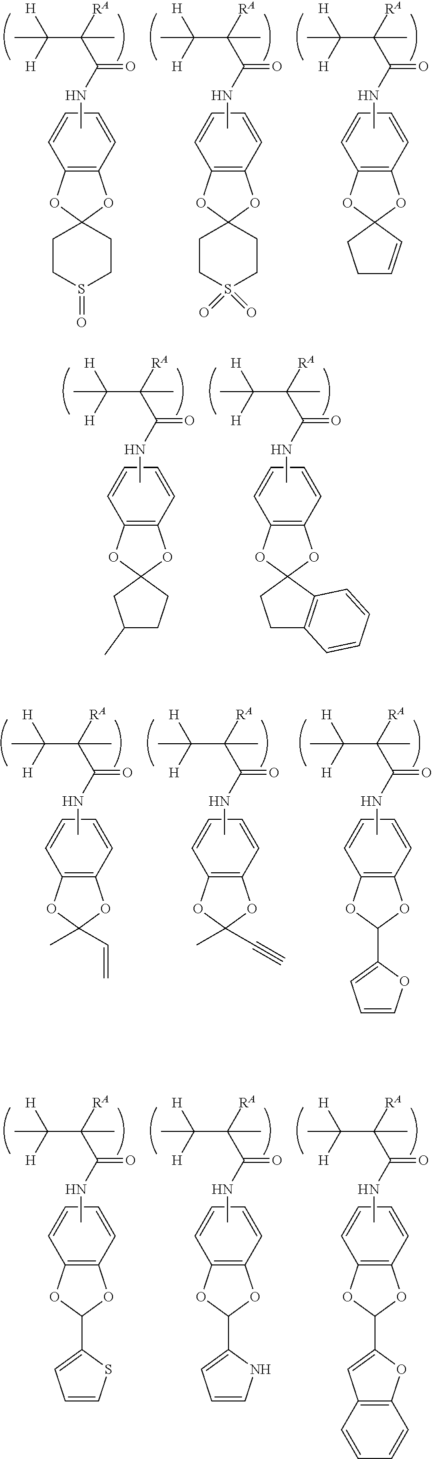

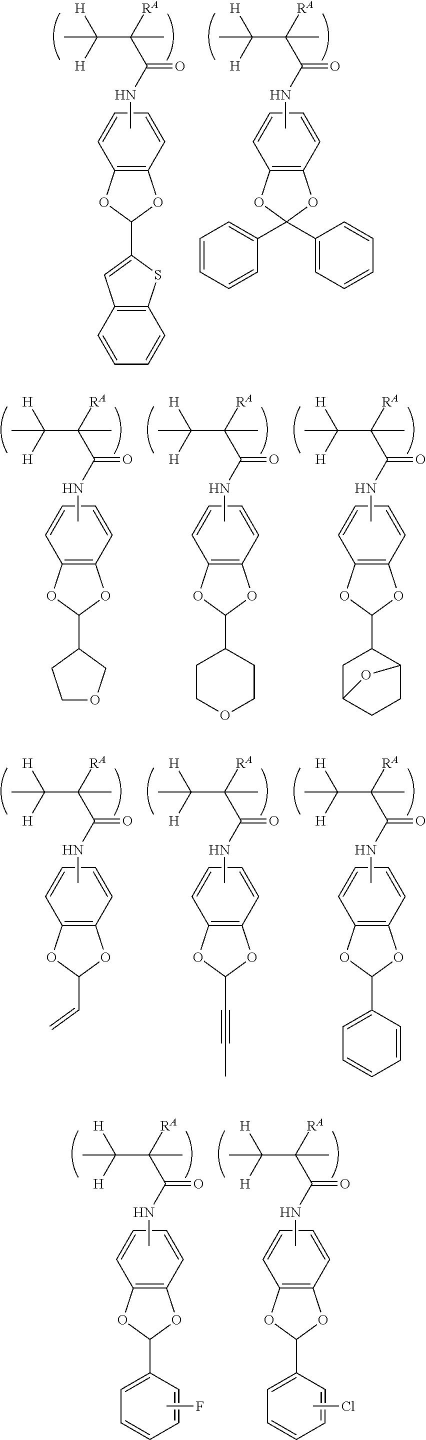

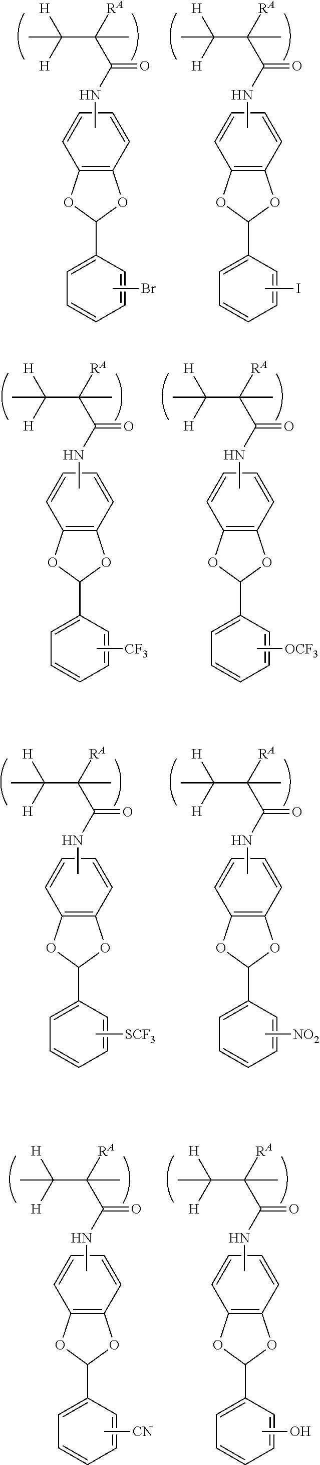

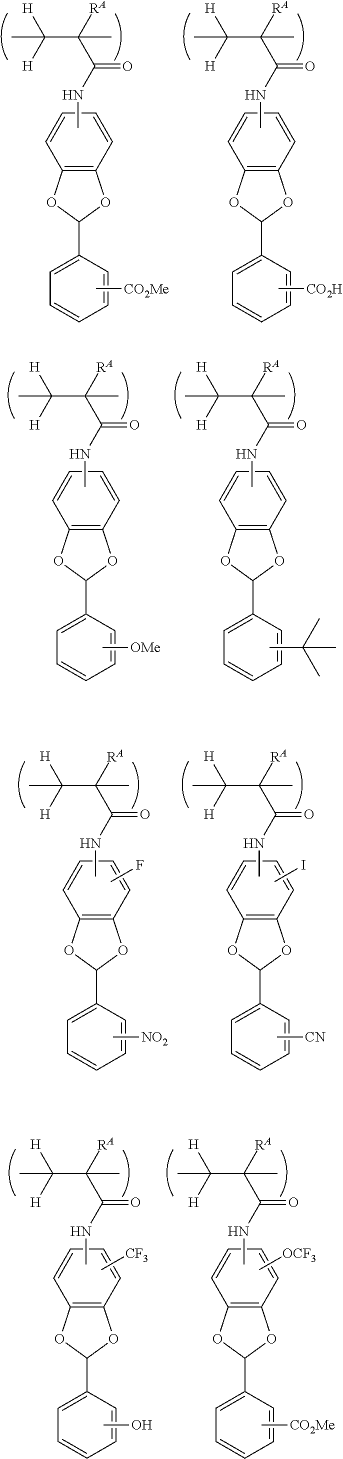

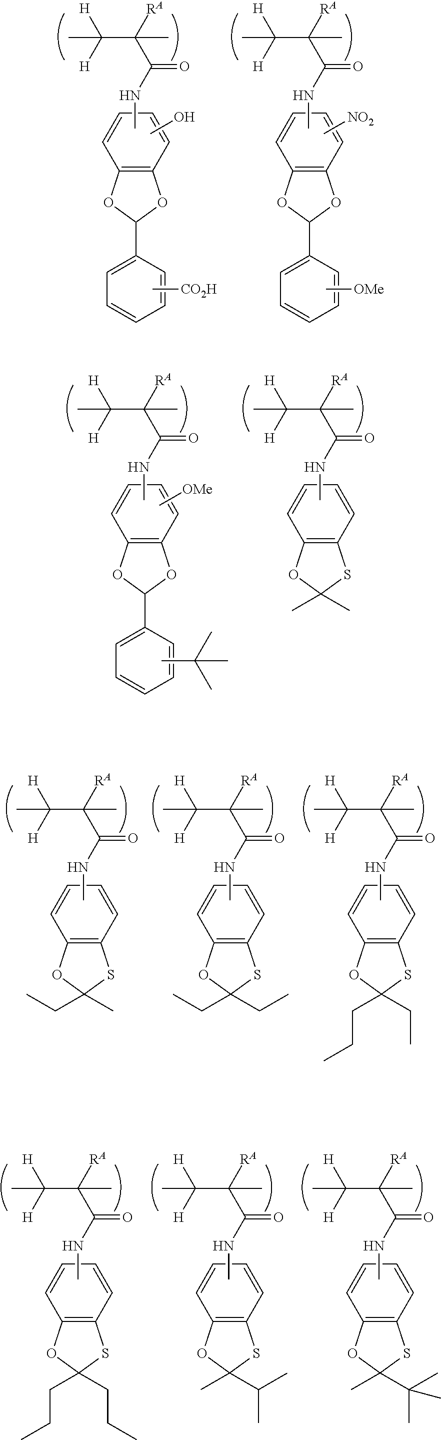

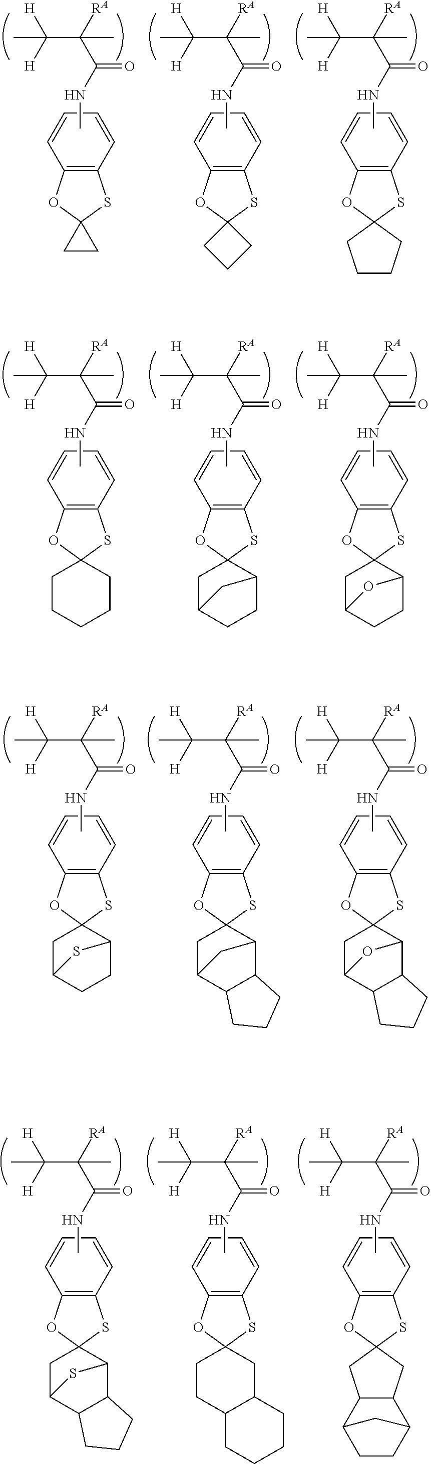

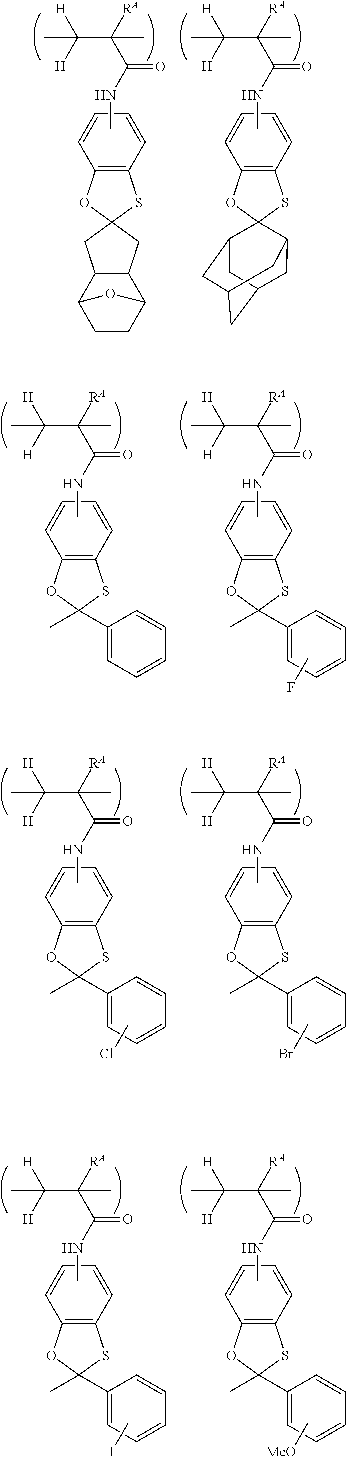

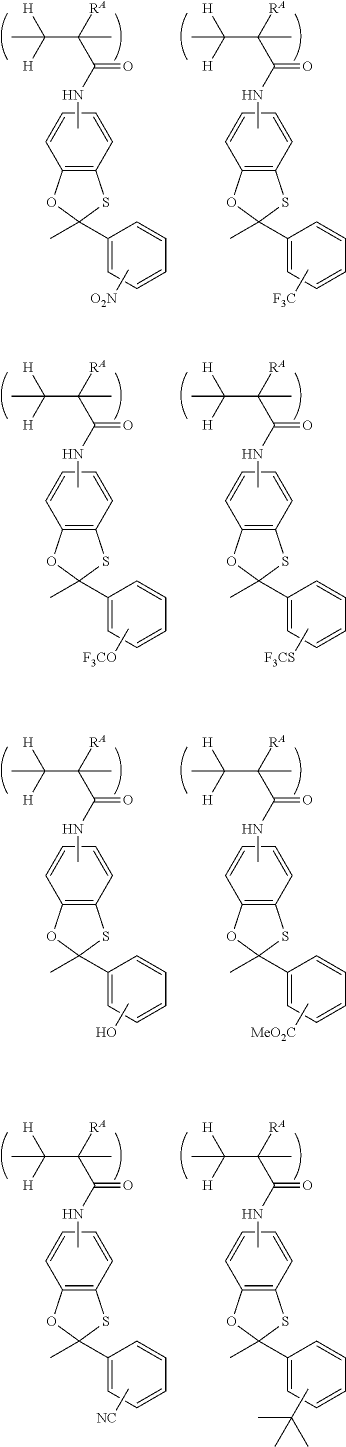

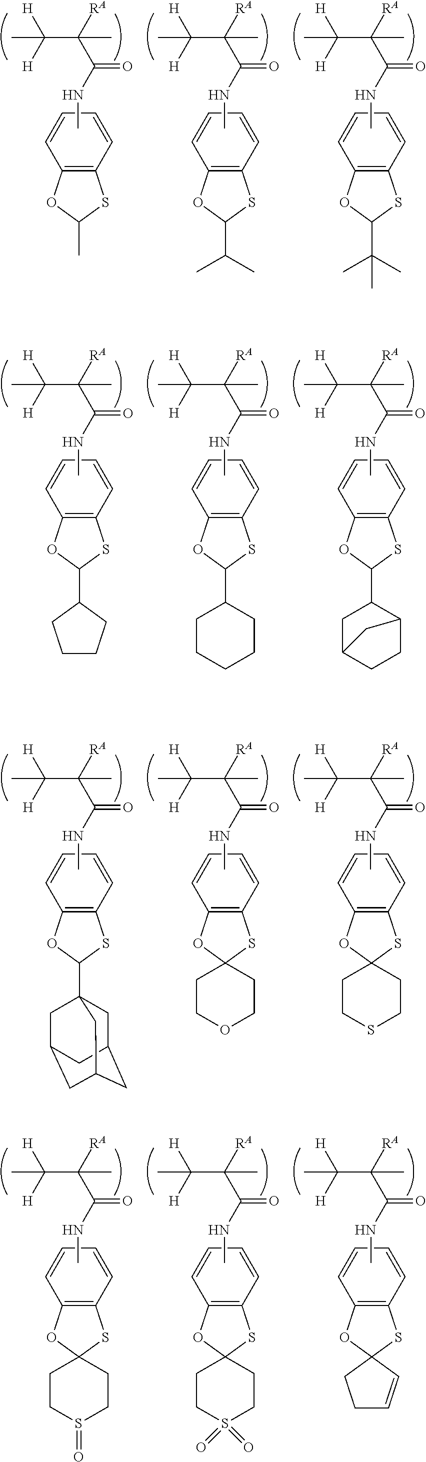

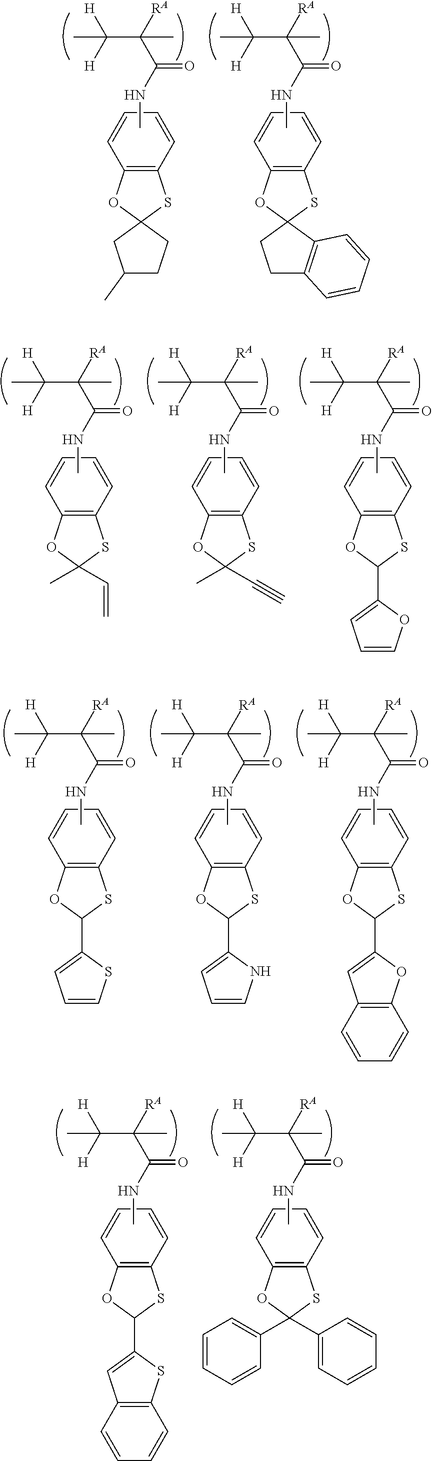

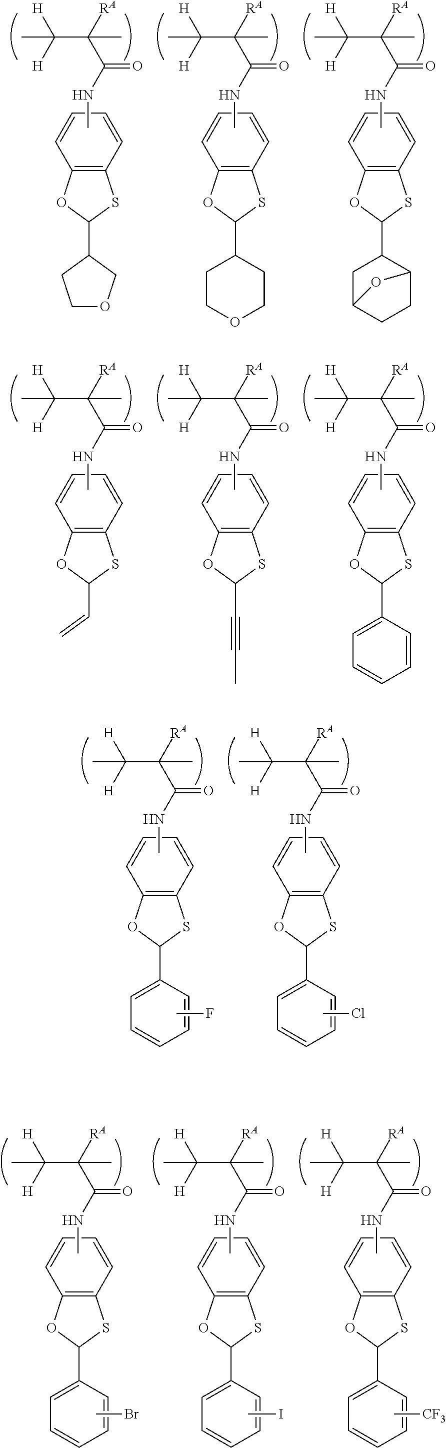

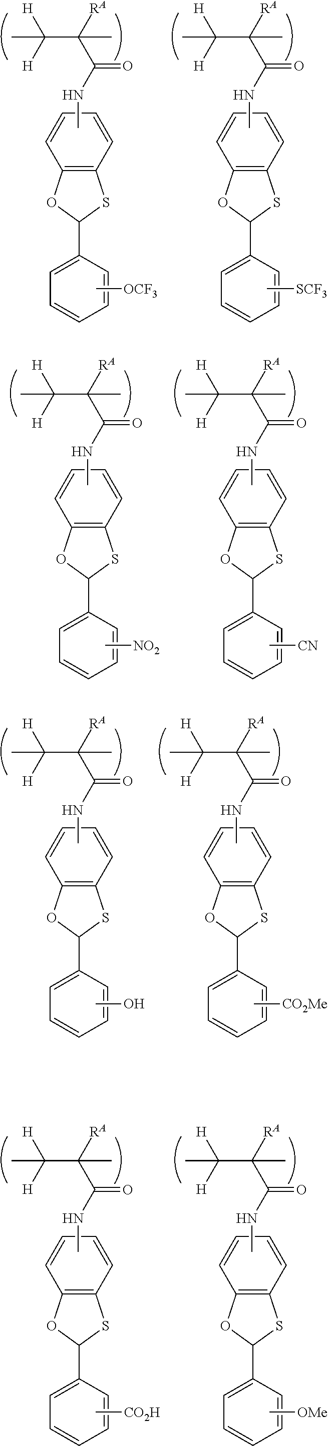

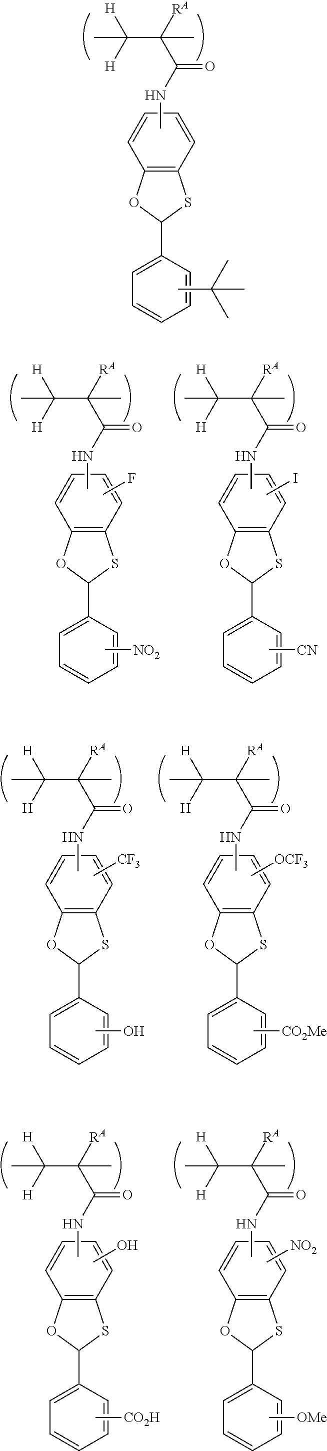

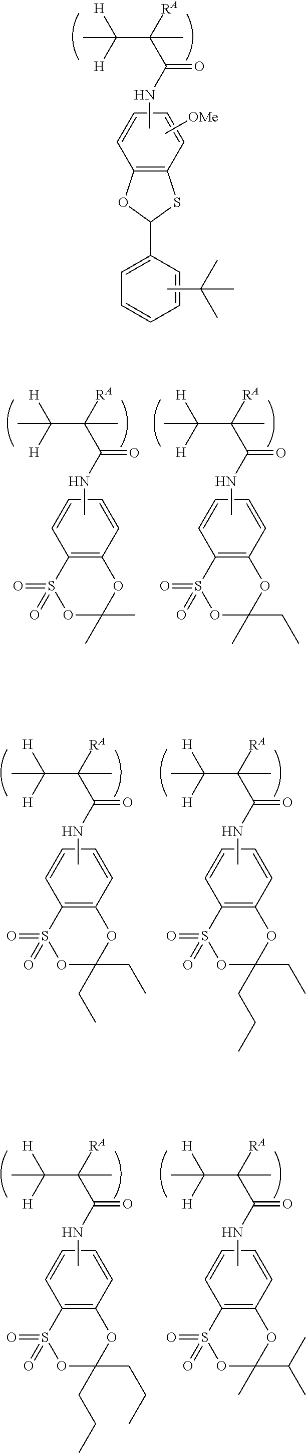

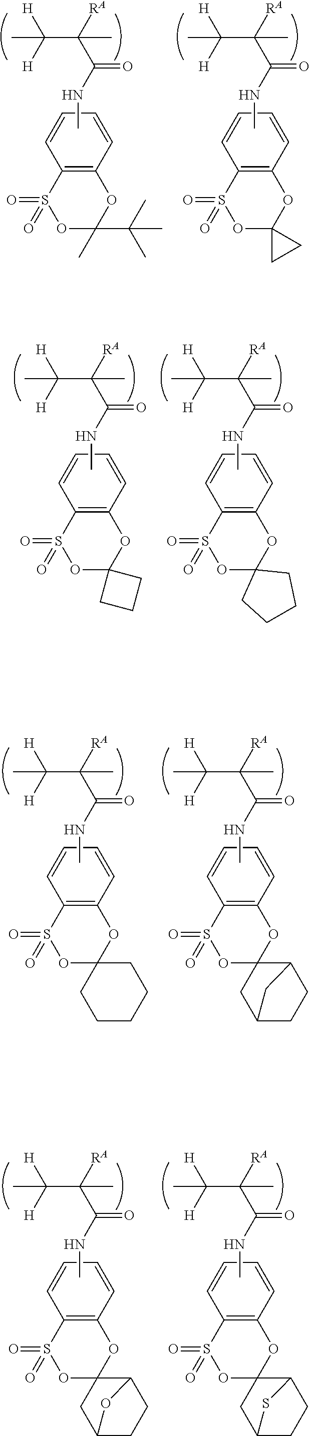

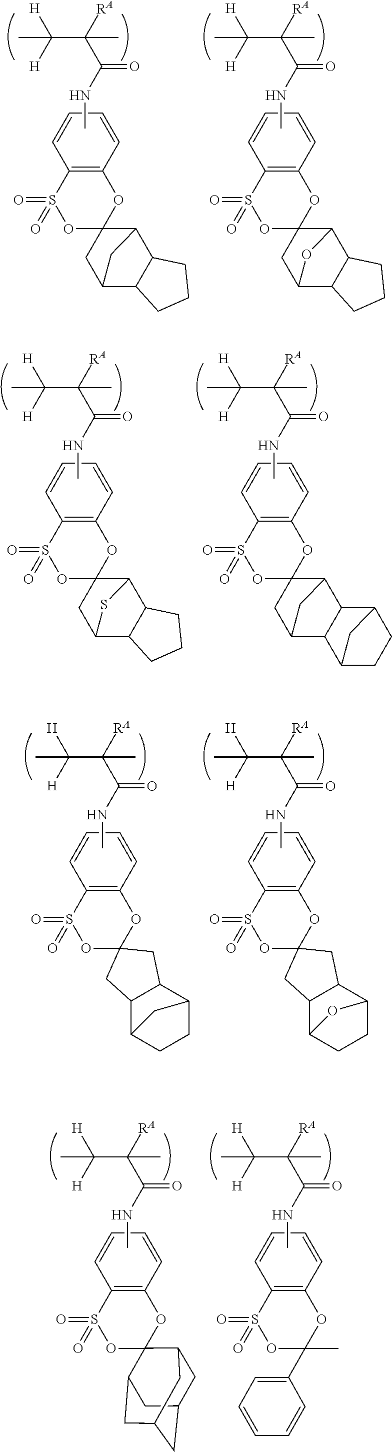

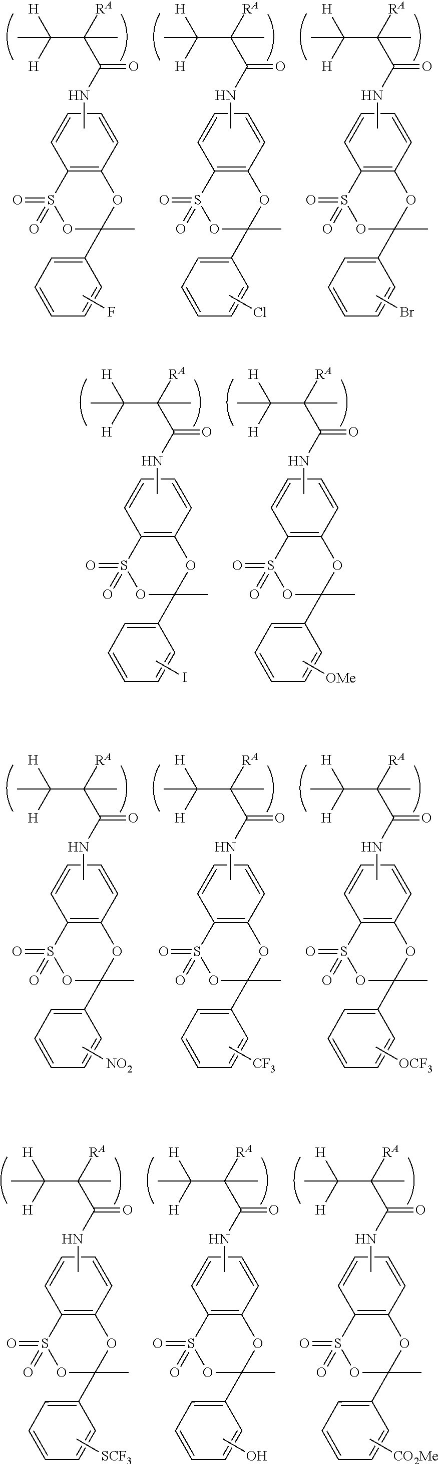

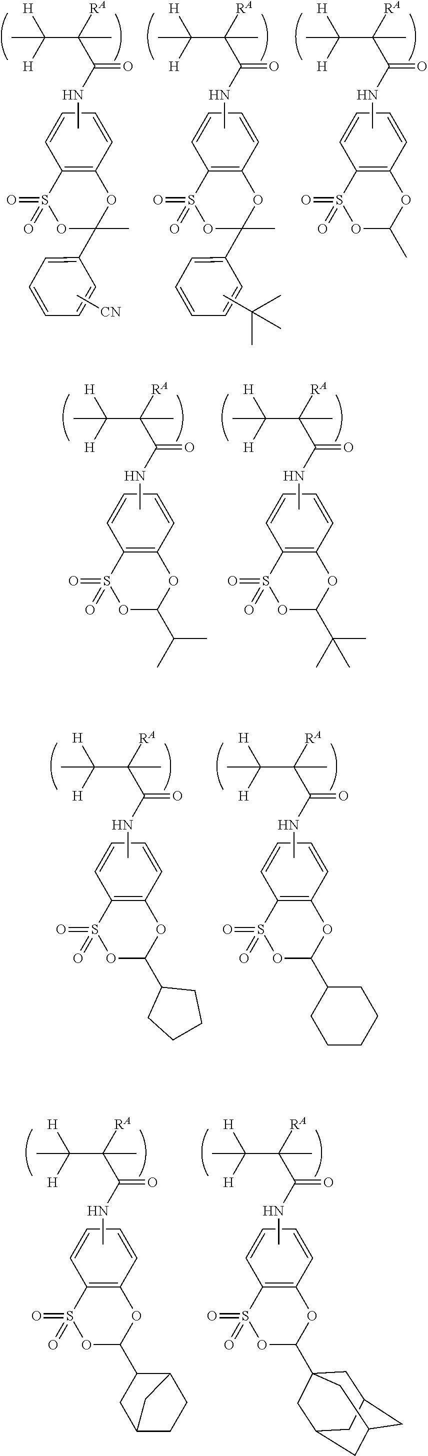

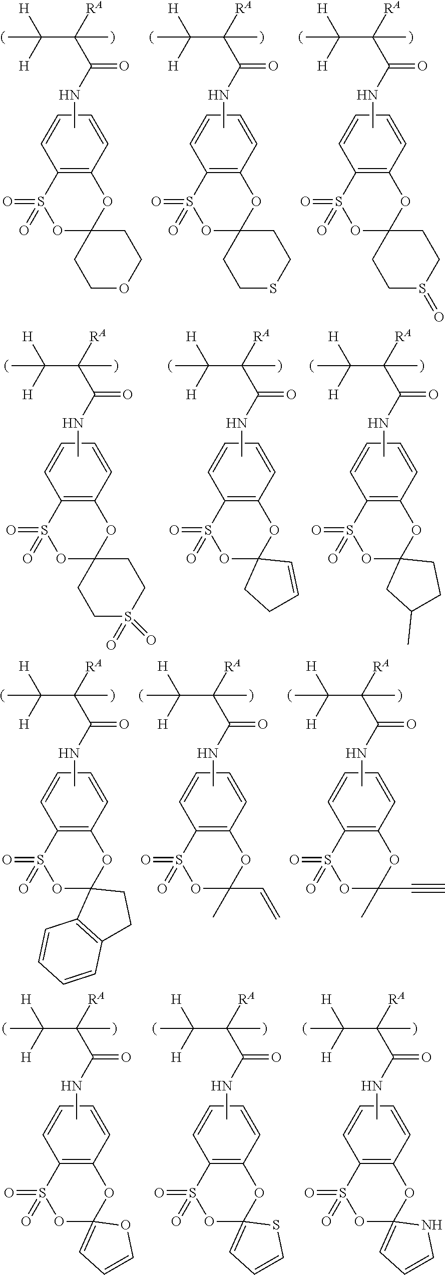

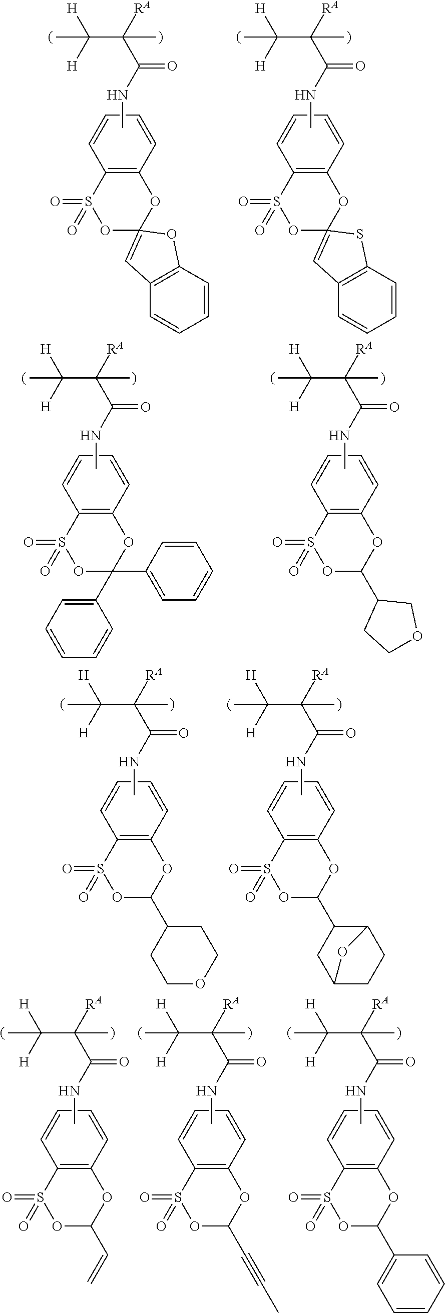

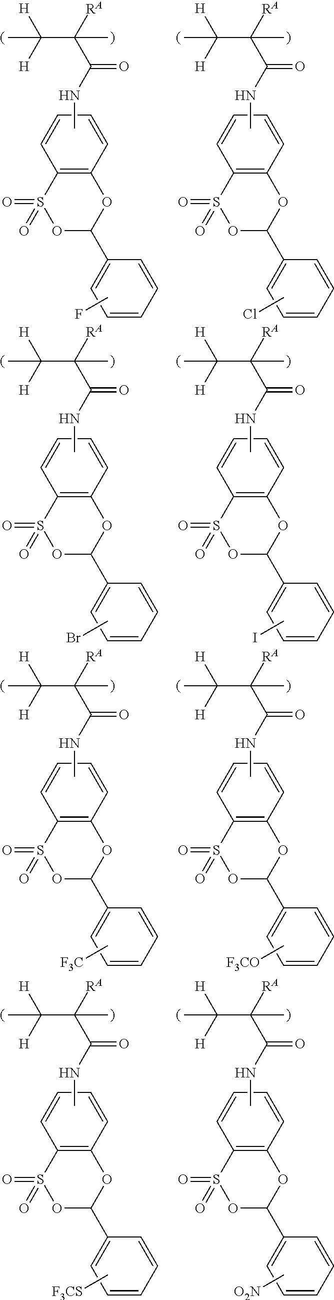

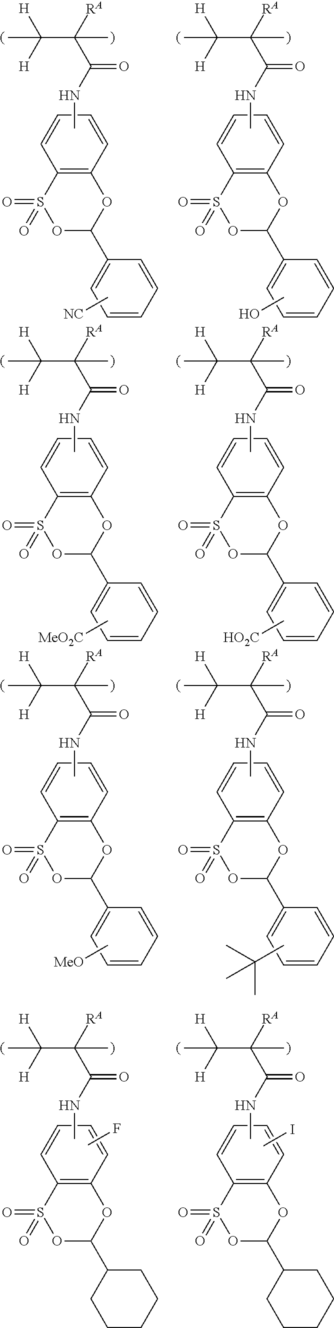

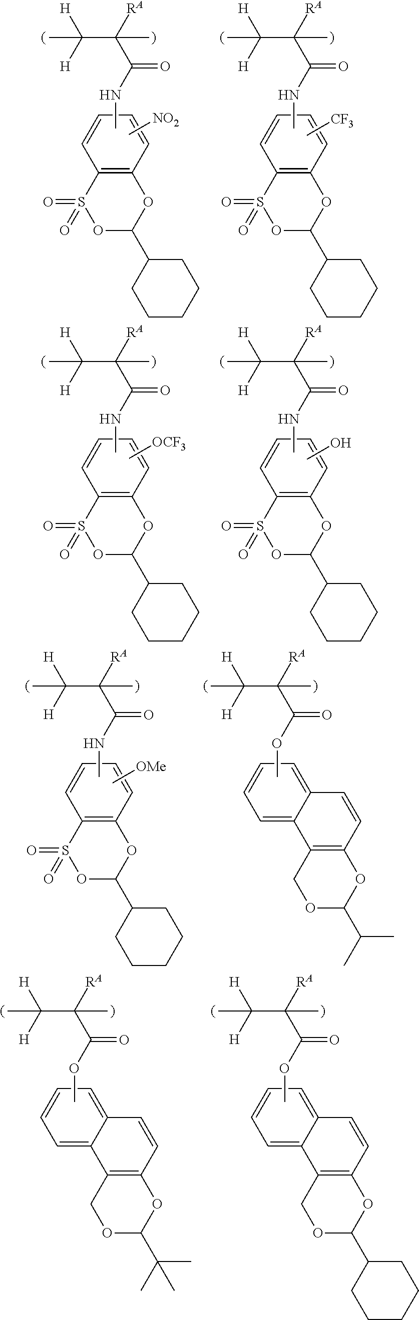

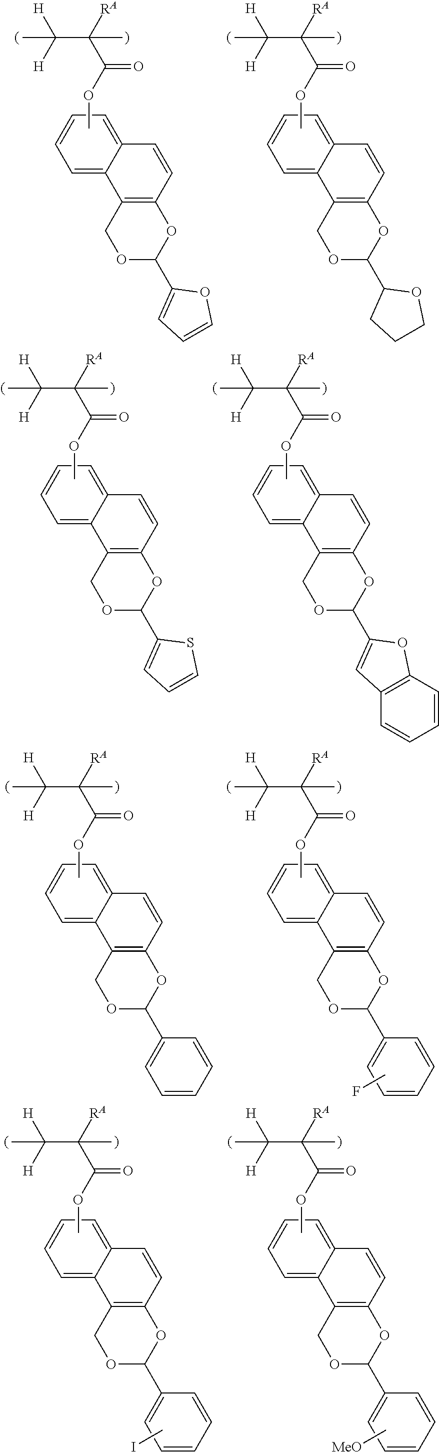

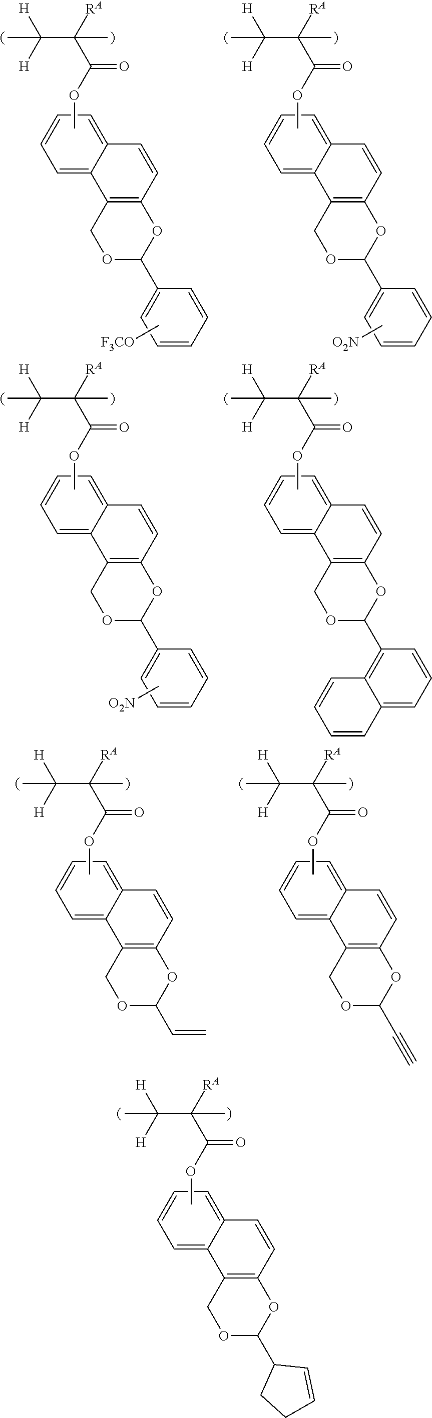

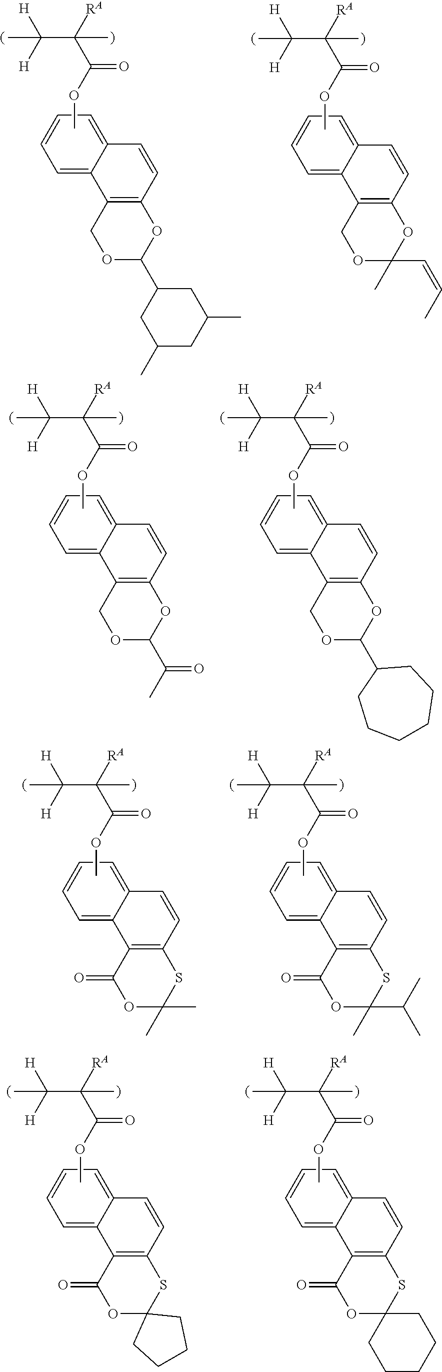

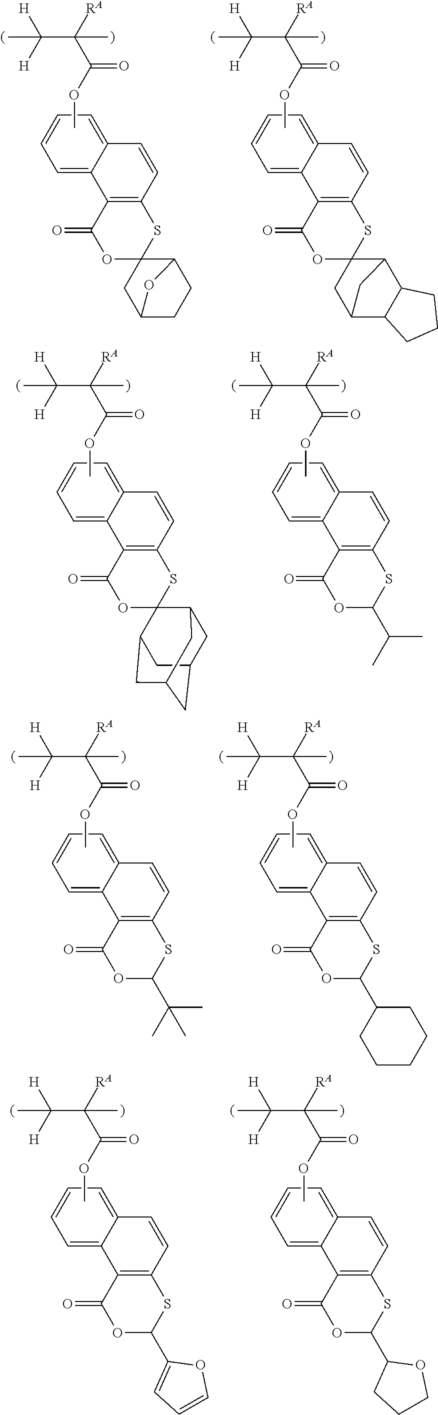

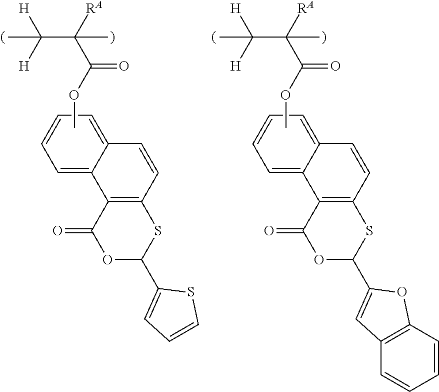

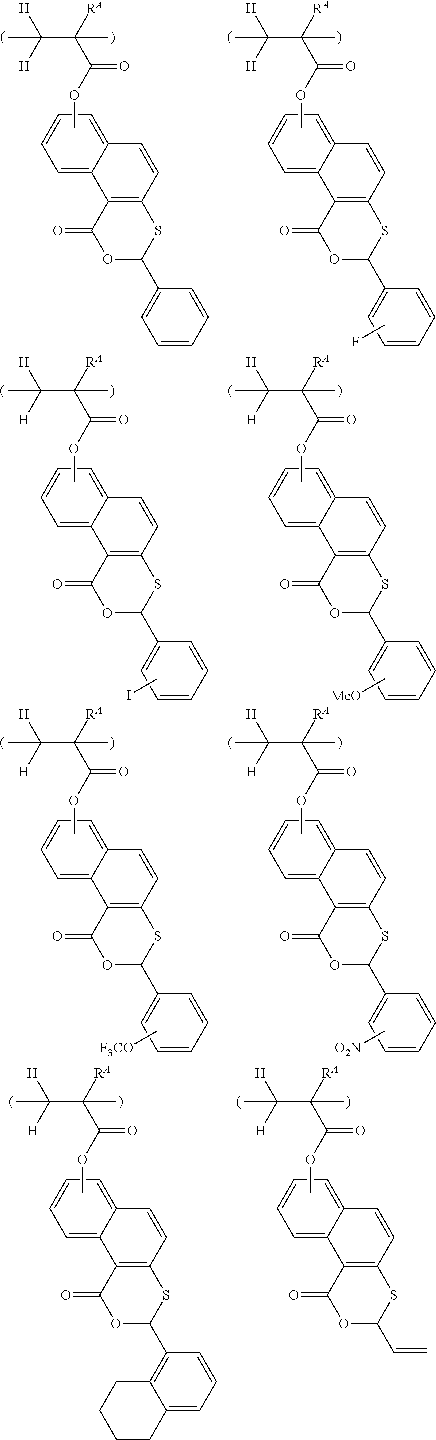

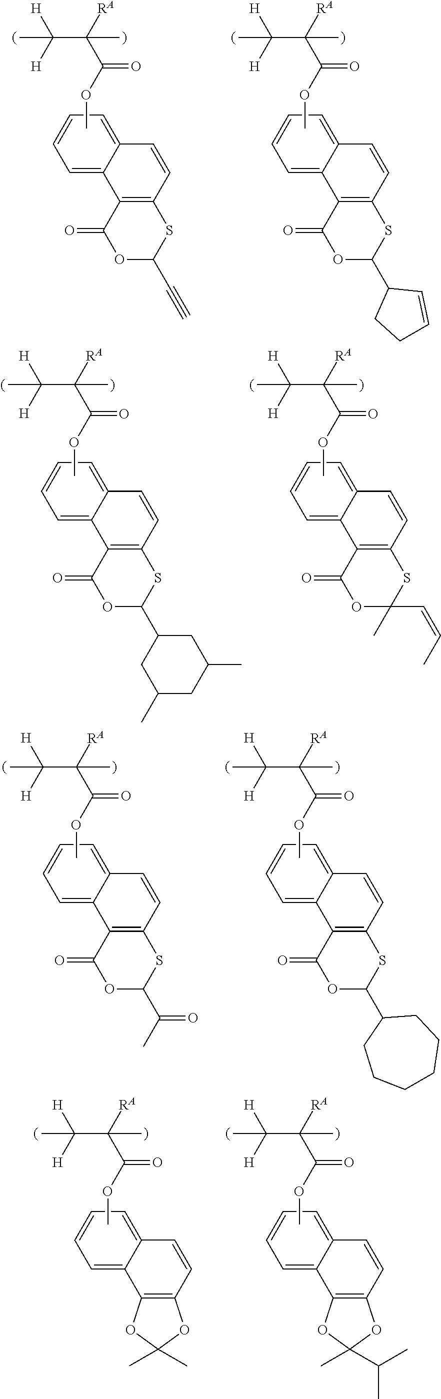

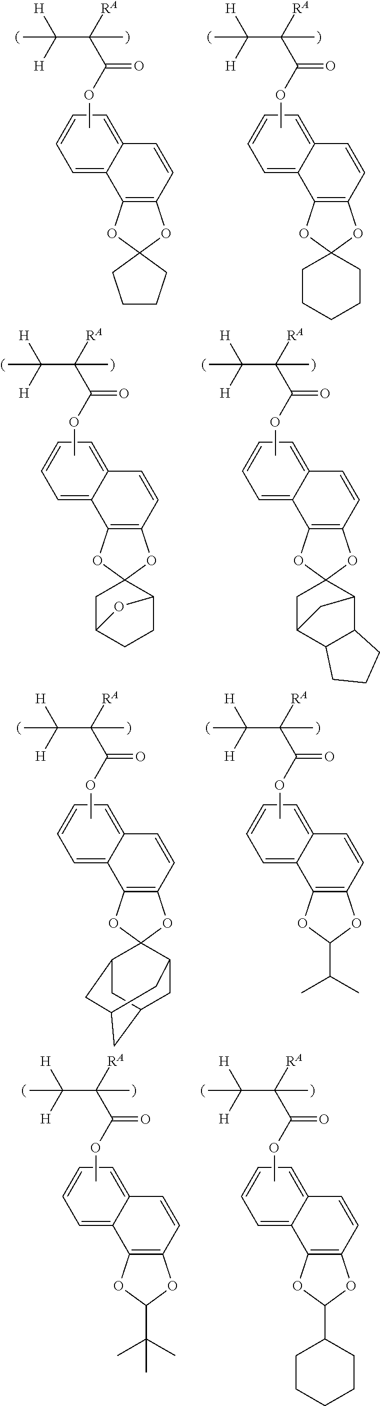

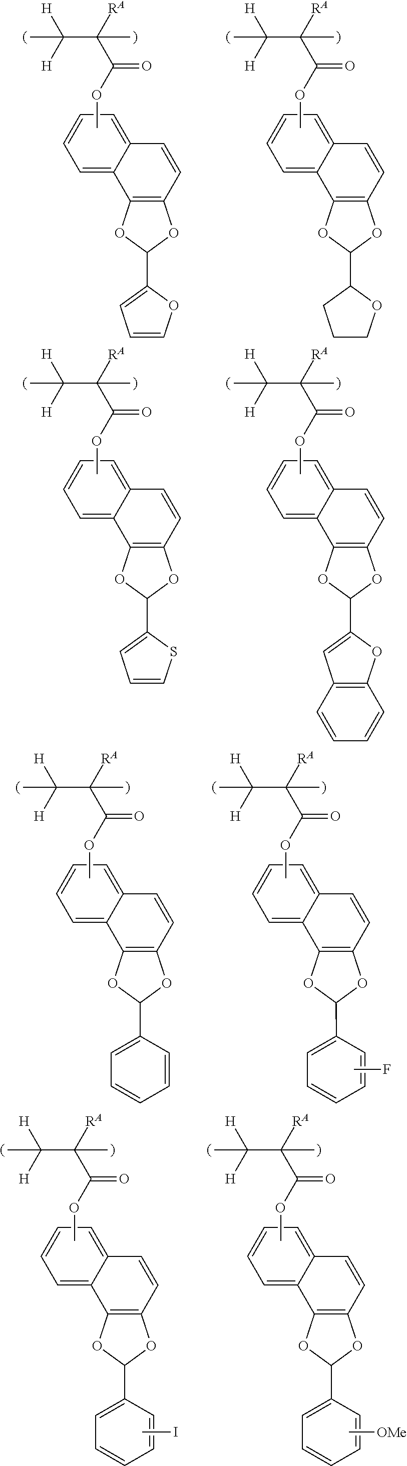

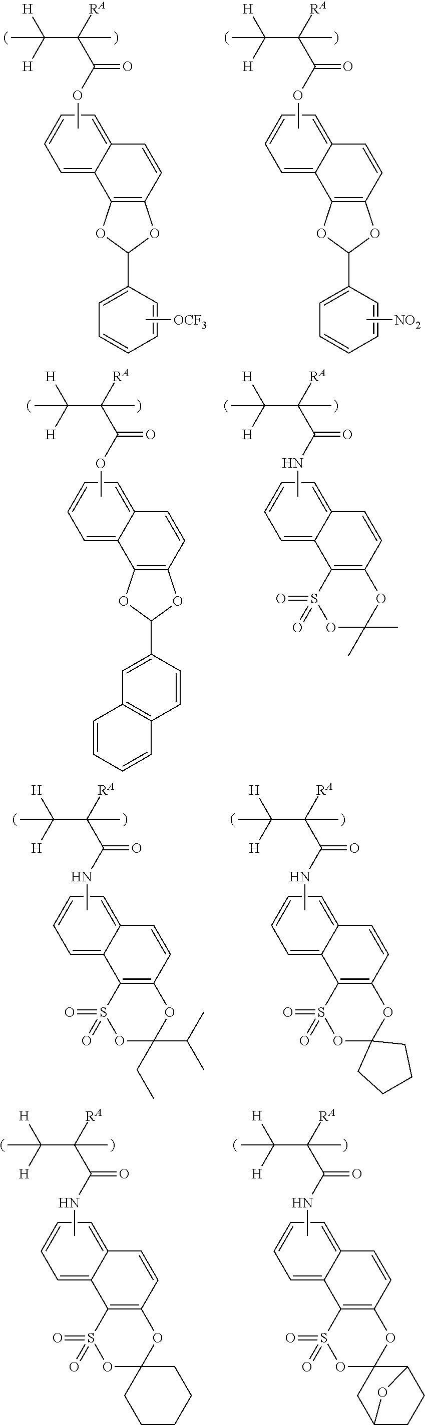

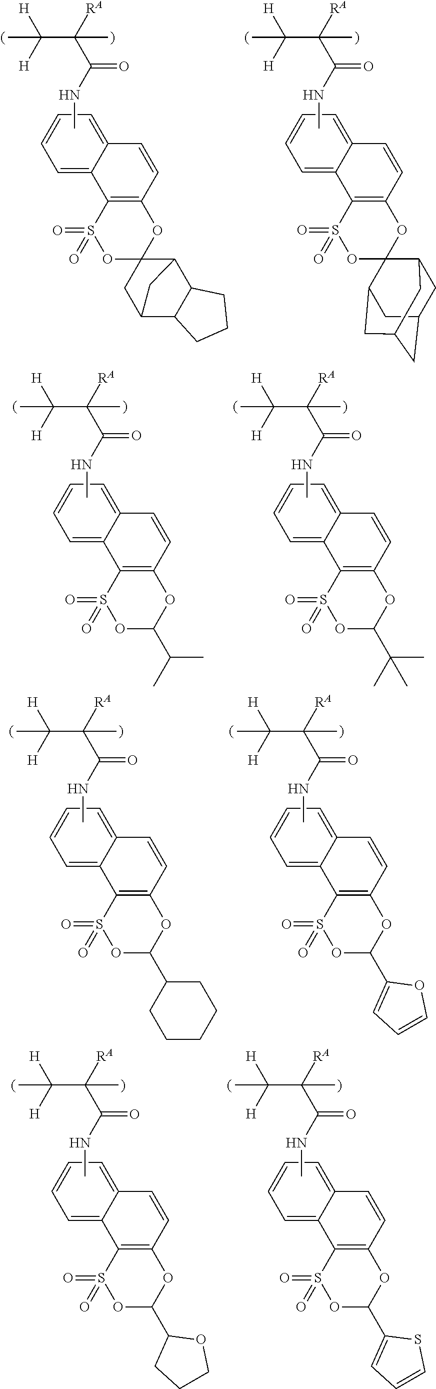

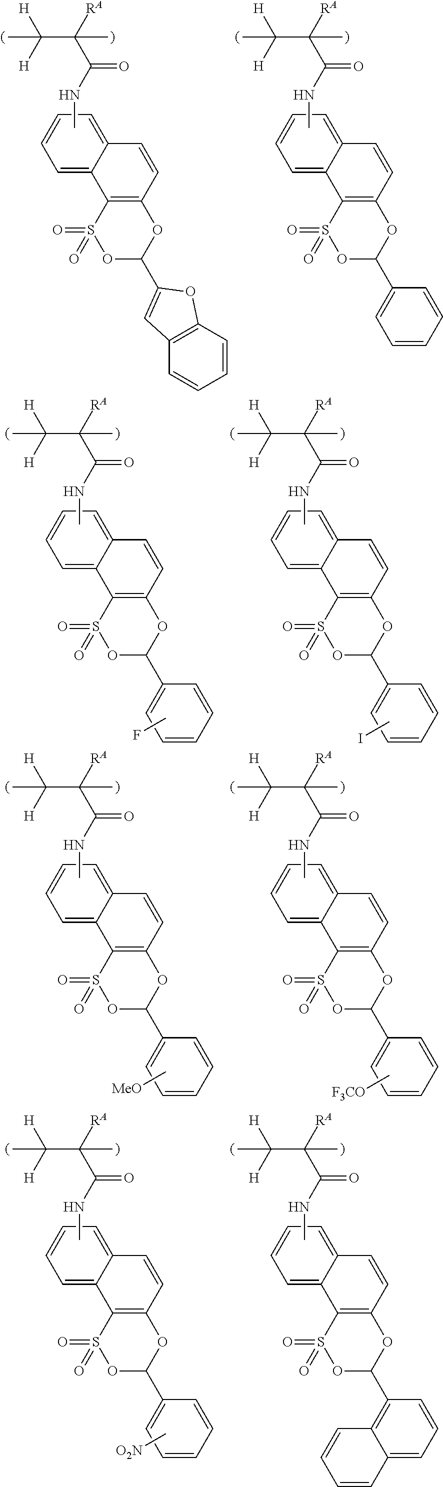

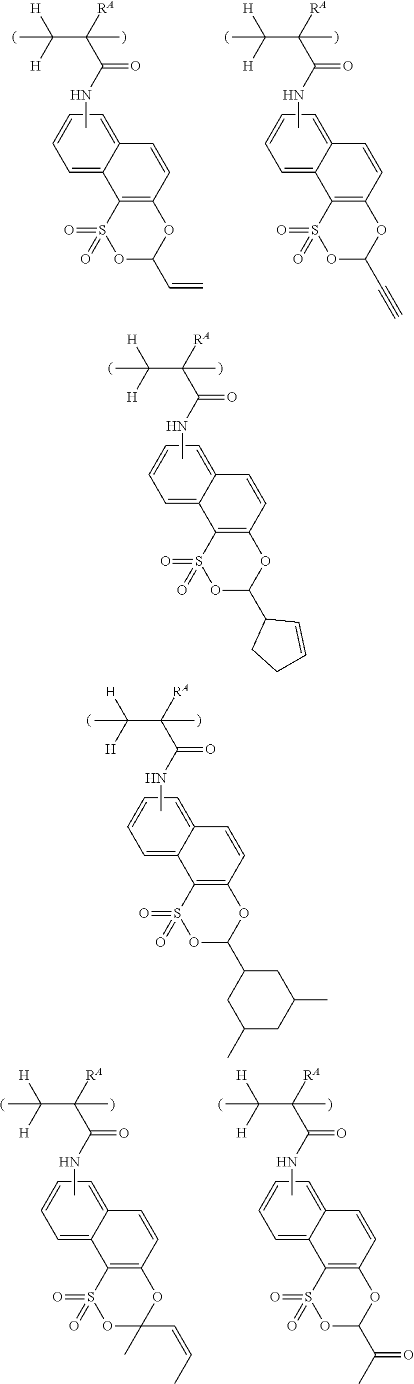

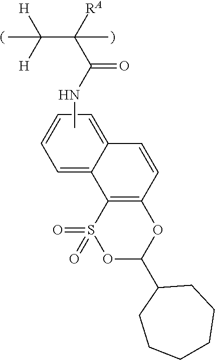

In a preferred embodiment, the polymer further comprises repeat units having the formula (c1), referred to as repeat units (c), hereinafter.

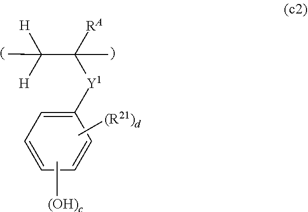

In formula (c1), R^{A} is hydrogen, fluorine, methyl or trifluoromethyl. Y¹ is a single bond, *—C(═O)—O—, or *—C(═O)—NH—, wherein * designates a point of attachment to the carbon atom in the backbone. R²¹ is halogen, nitro, cyano, carboxy, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom, c is an integer of 1 to 4, d is an integer of 0 to 3, and c+d is from 1 to 5.

Examples of the repeat unit (c) are shown below, but not limited thereto. Herein R^{A} is as defined above.

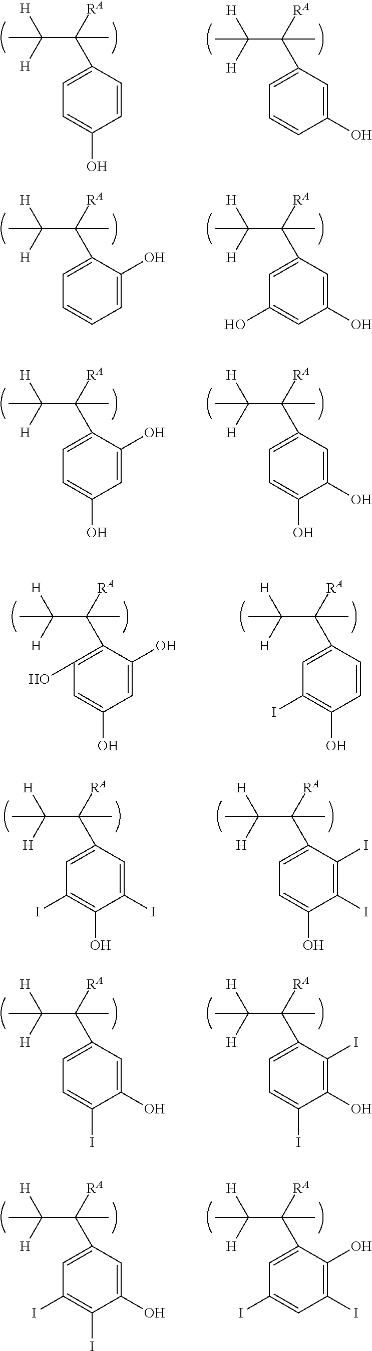

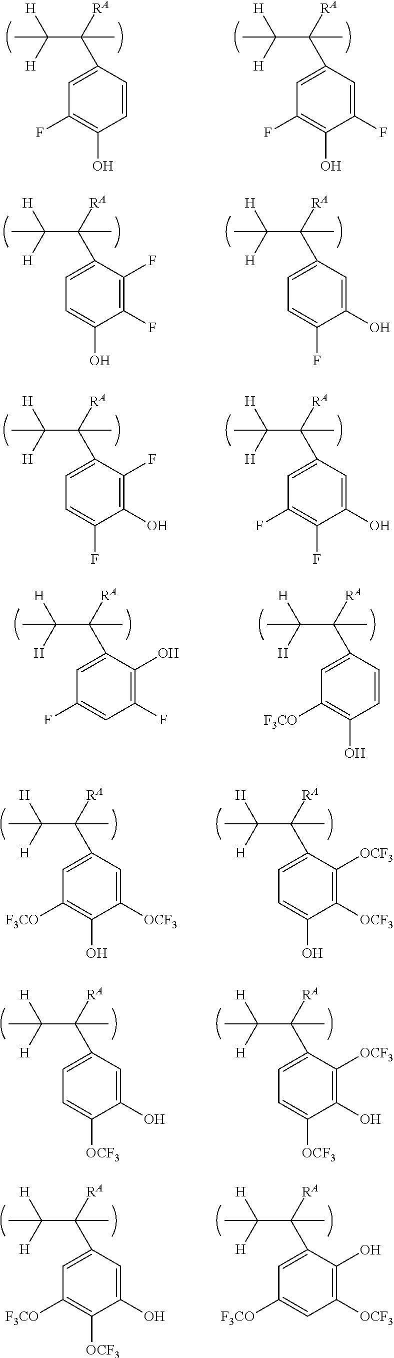

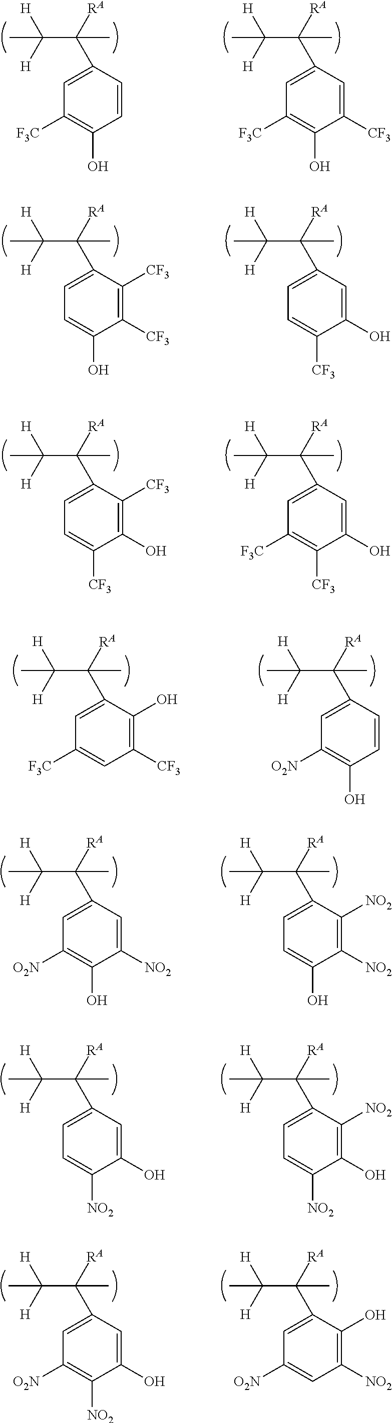

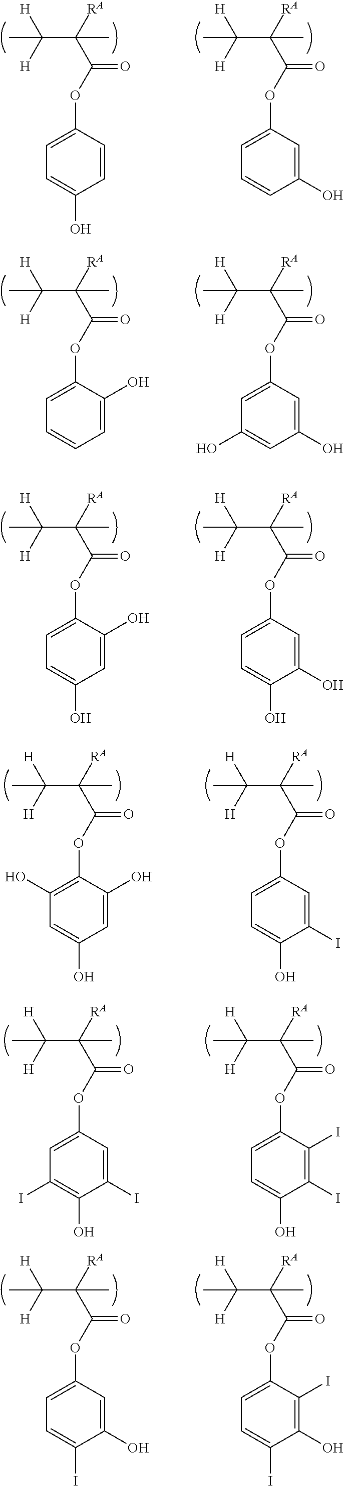

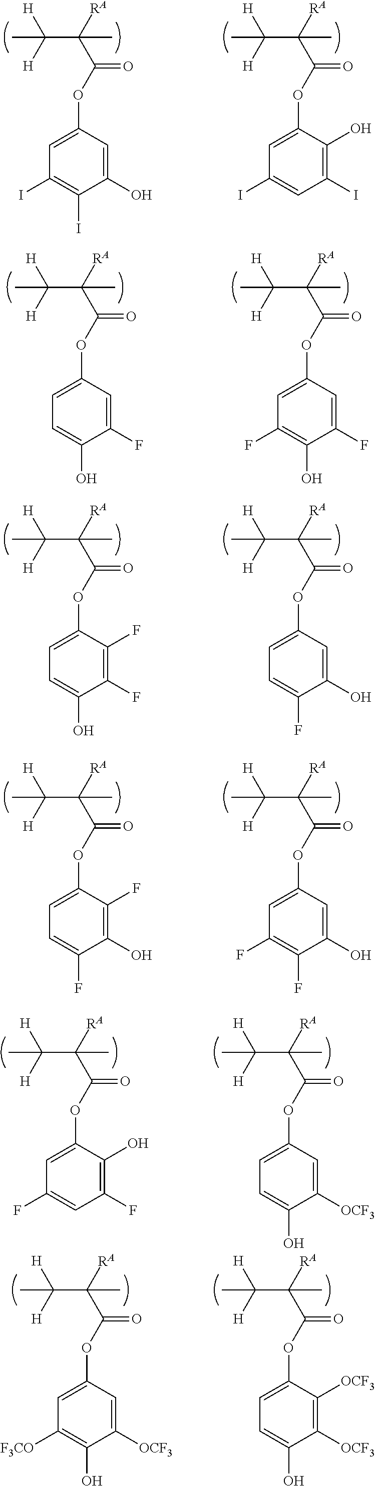

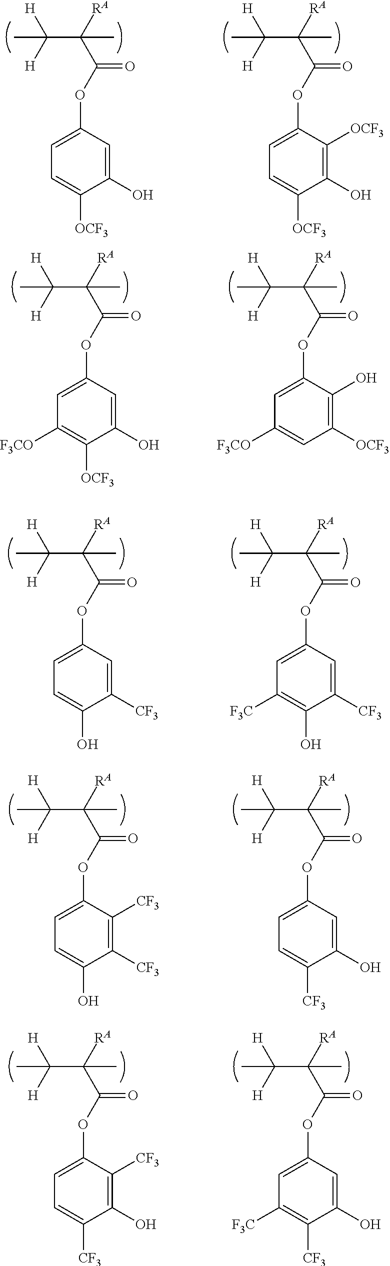

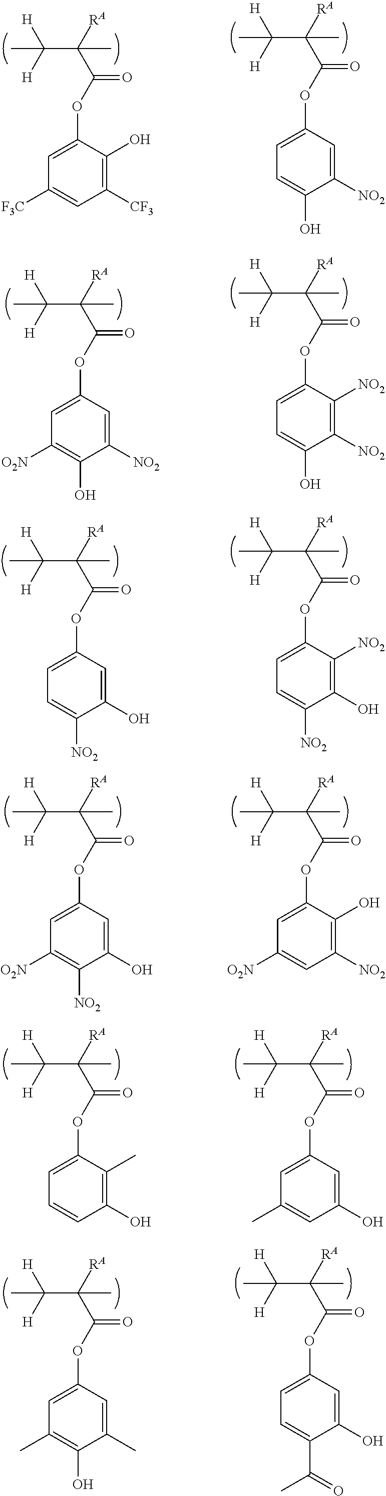

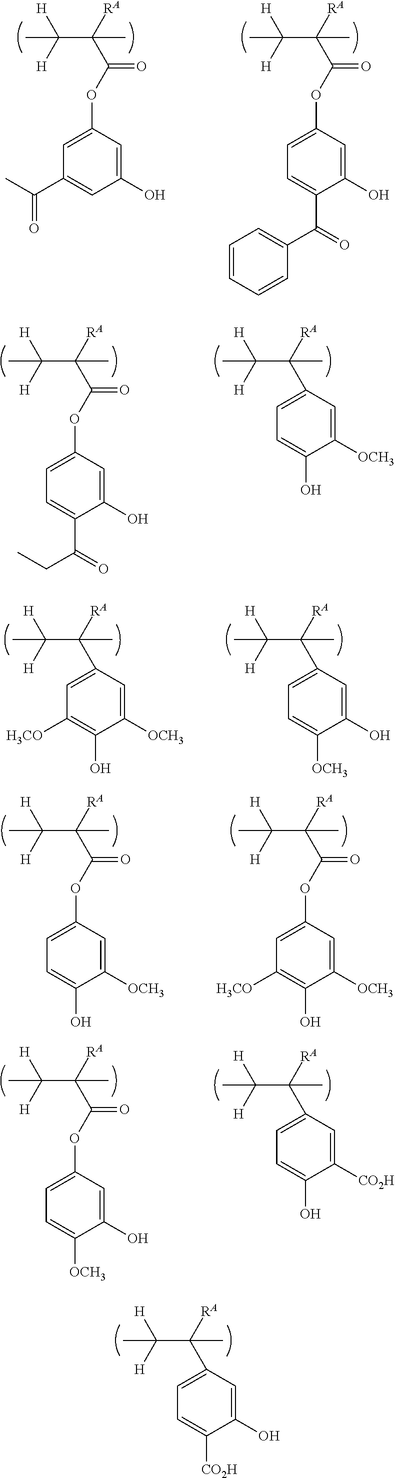

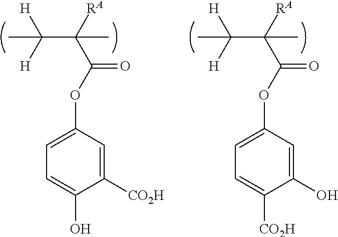

The polymer may further comprise repeat units having the formula (dl), which are also referred to as repeat units (d).

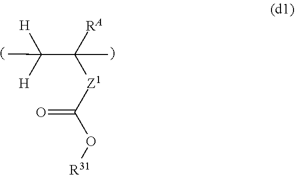

In formula (dl), R^{A} is hydrogen, fluorine, methyl or trifluoromethyl. Z¹ is a single bond, phenylene, naphthylene, *—C(═O)—O—Z¹¹—, or *—C(═O)—NH—Z¹¹—. The phenylene or naphthylene group may be substituted with hydroxy, nitro, cyano, an optionally fluorinated C₁-C₁₀ alkoxy moiety or halogen. The asterisk (*) designates a point of attachment to the carbon atom in the backbone. Z¹¹ is a C₁-C₁₀ saturated hydrocarbylene group, phenylene, or naphthylene. The saturated hydrocarbylene group may contain a hydroxy, ether bond, ester bond or lactone ring. R³¹ is hydrogen or a C₁-C₂₀ group which contains at least one structure selected from hydroxy other than phenolic hydroxy, cyano, carbonyl, carboxy, ether bond, ester bond, sulfonate ester bond, carbonate bond, lactone ring, sultone ring and carboxylic anhydride (—C(═O)—O—C(═O)—).

Examples of the repeat unit (d) are shown below, but not limited thereto. Herein R^{A} is as defined above.

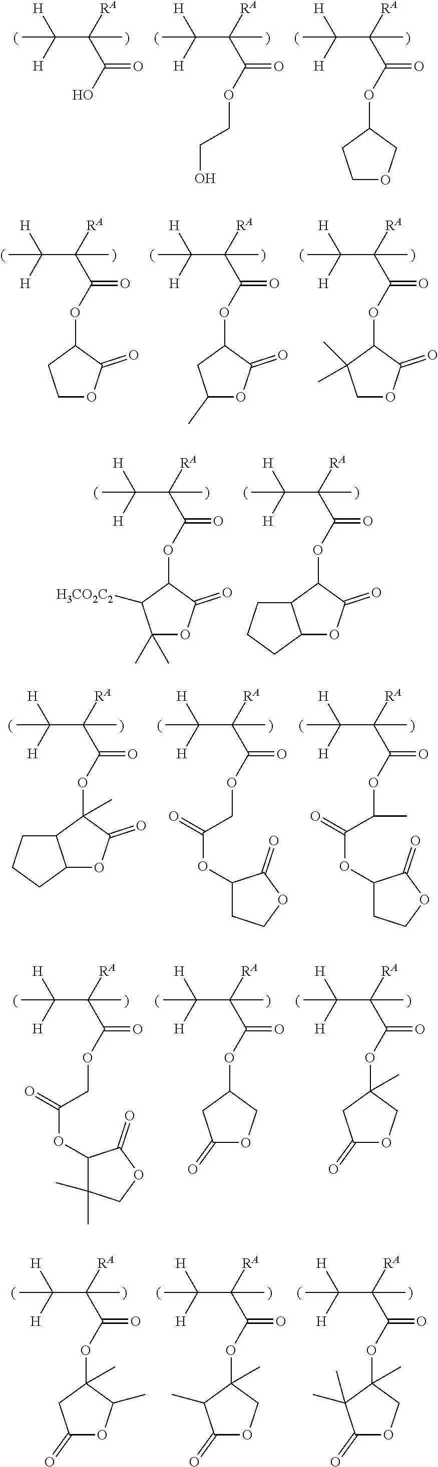

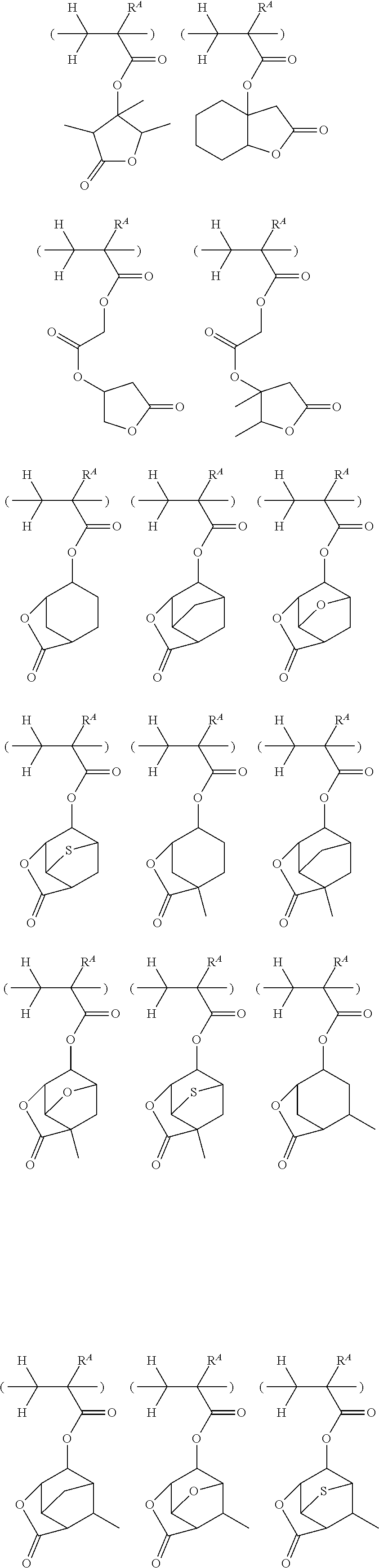

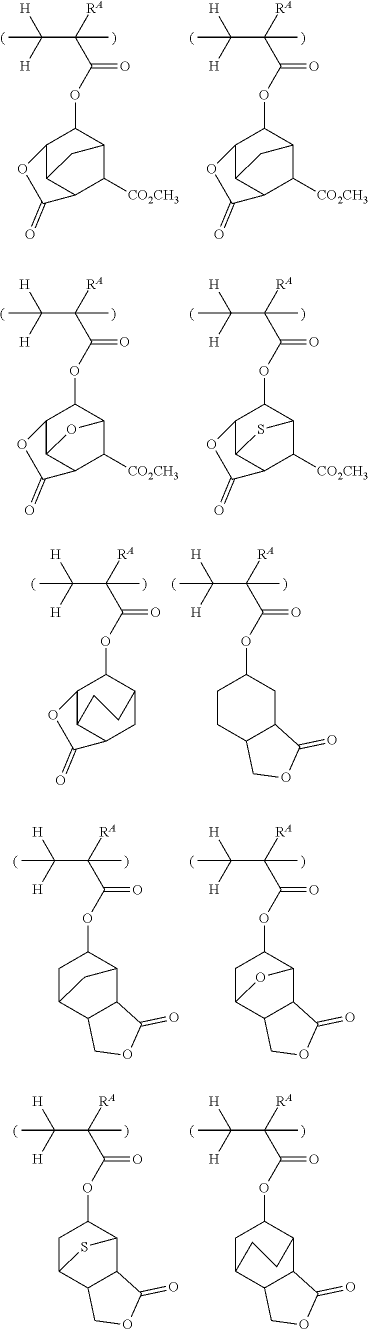

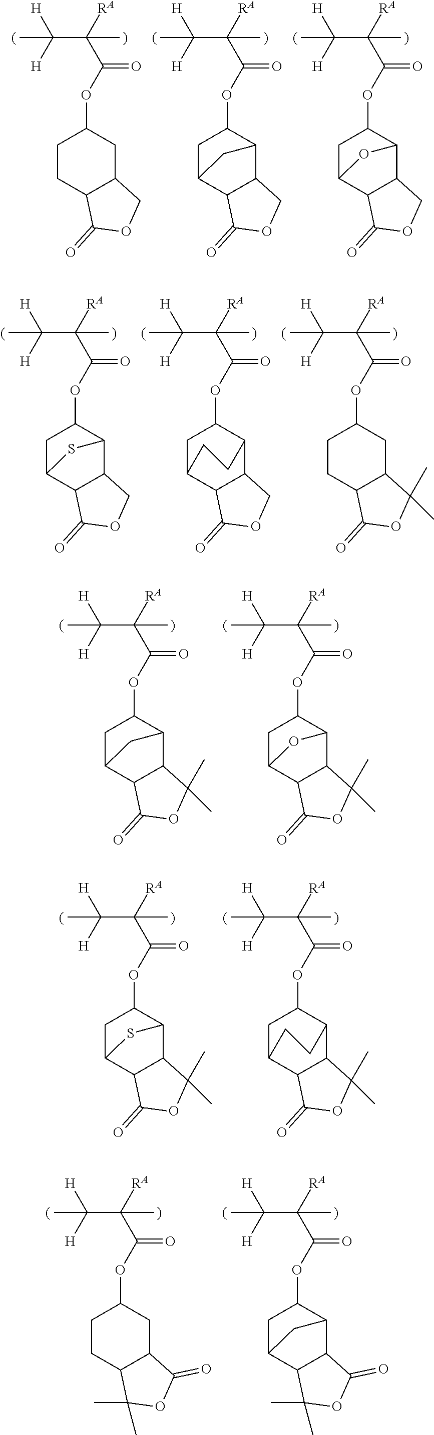

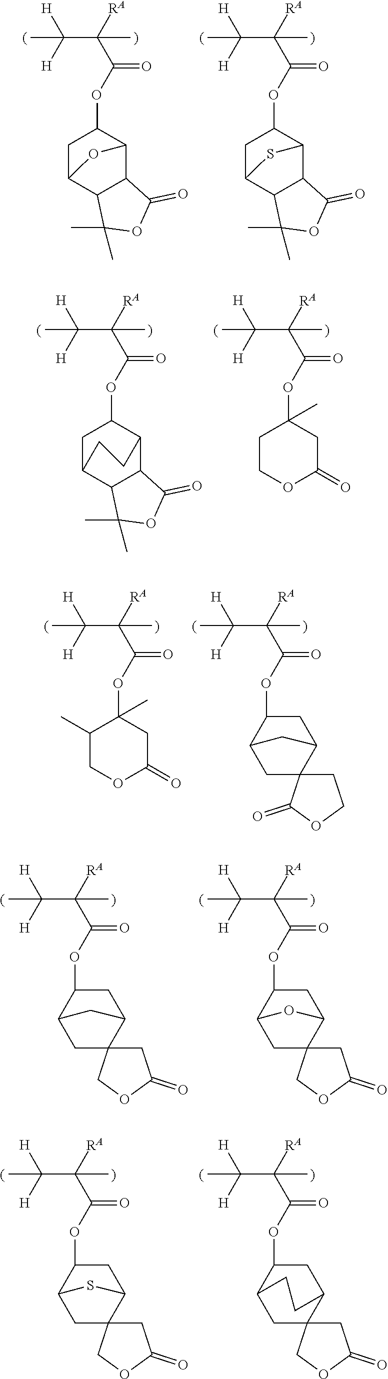

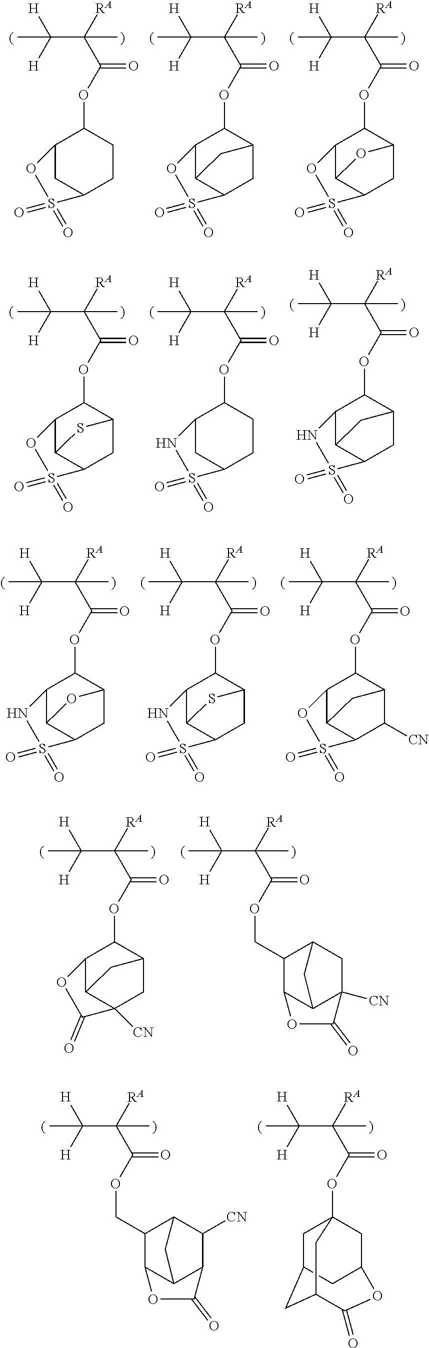

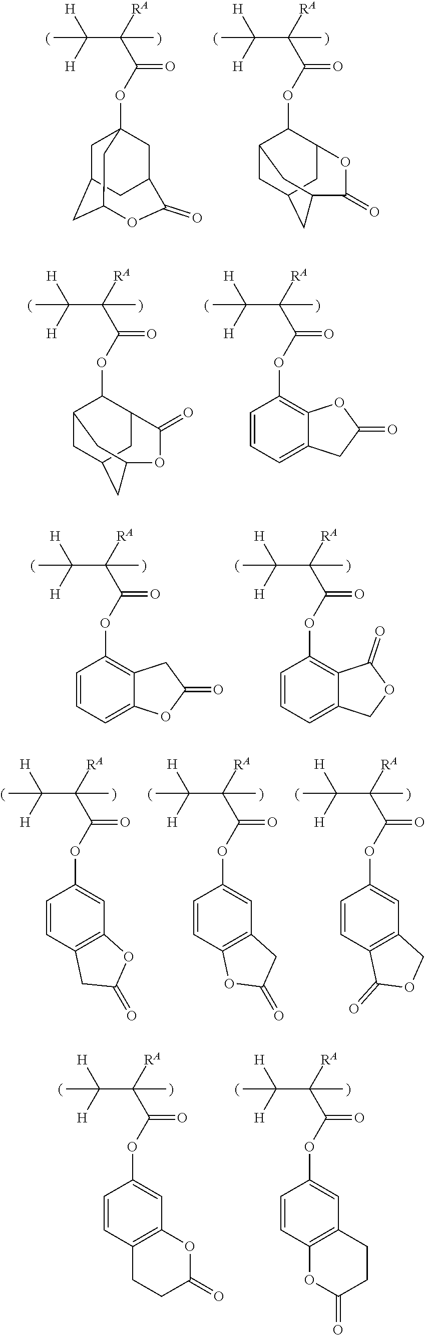

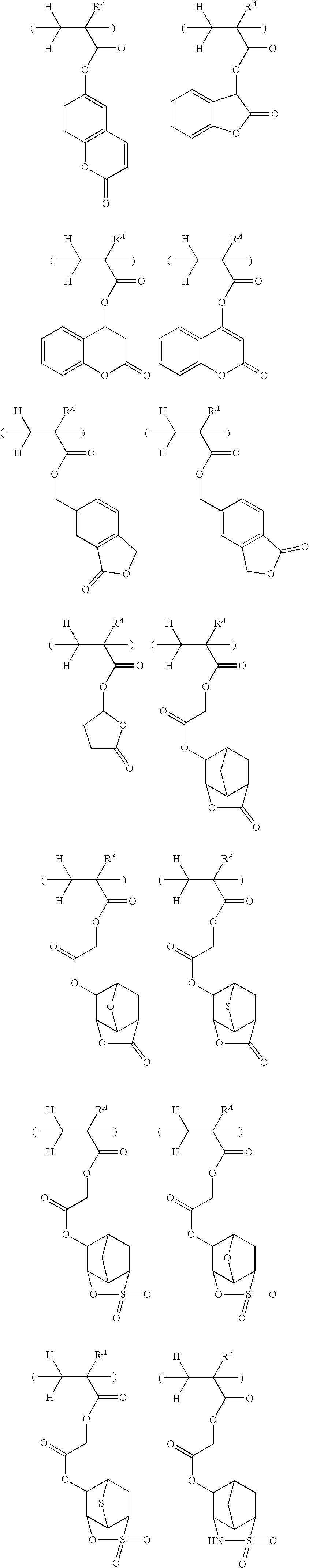

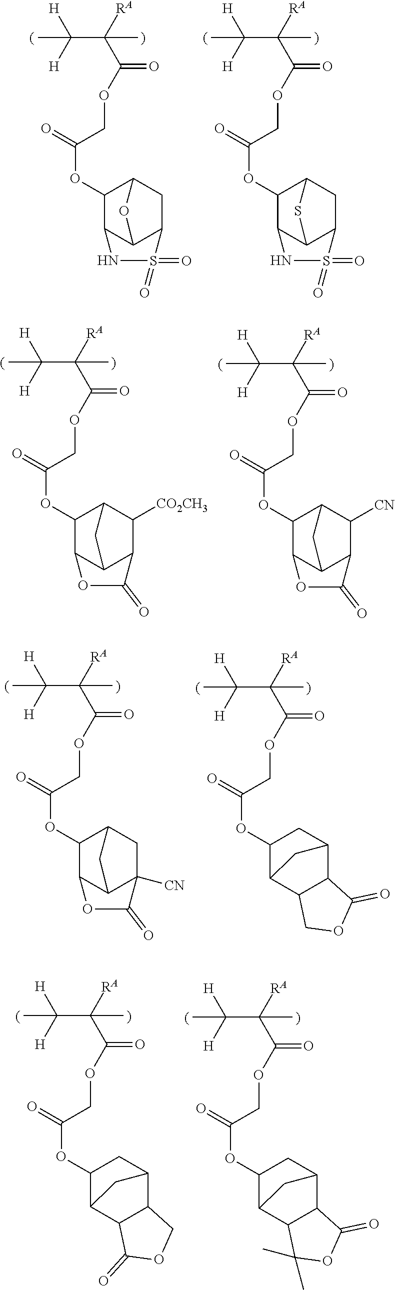

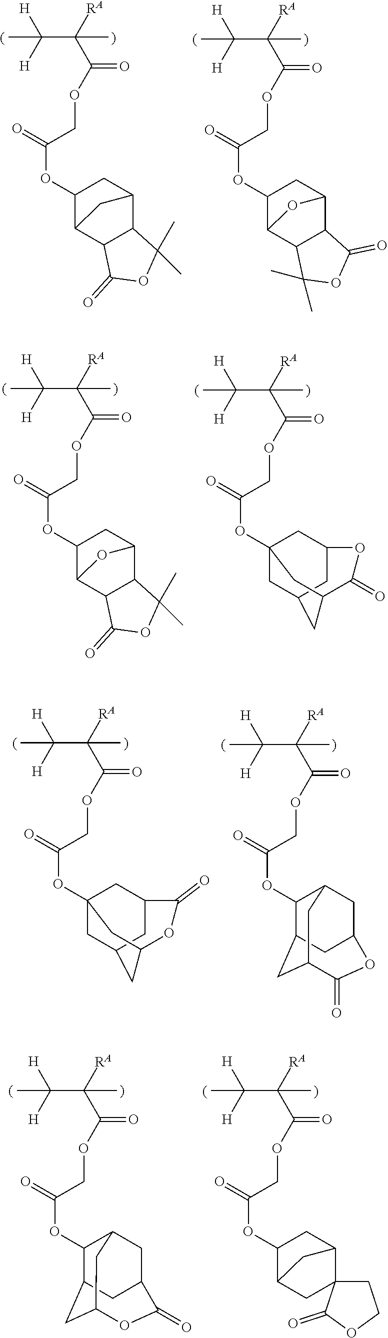

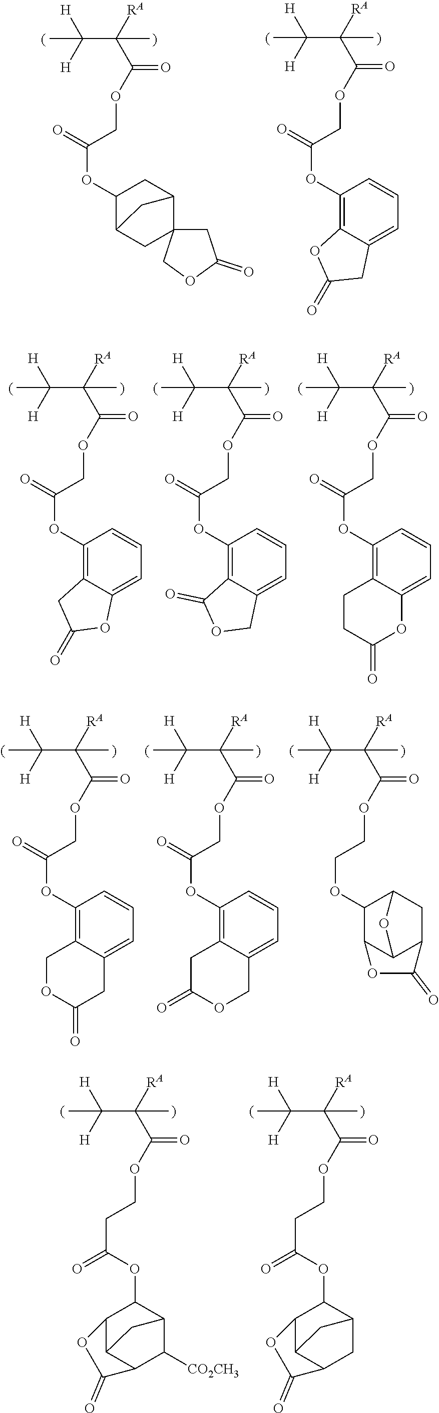

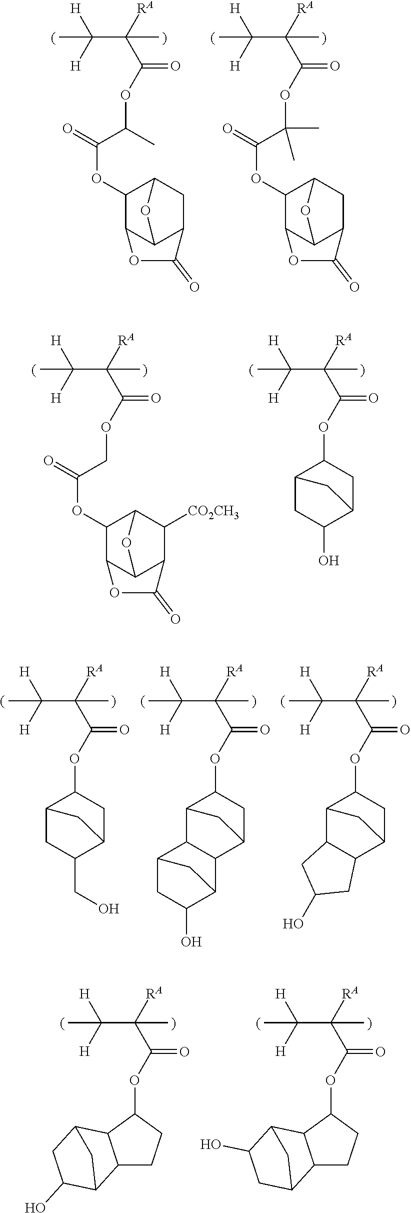

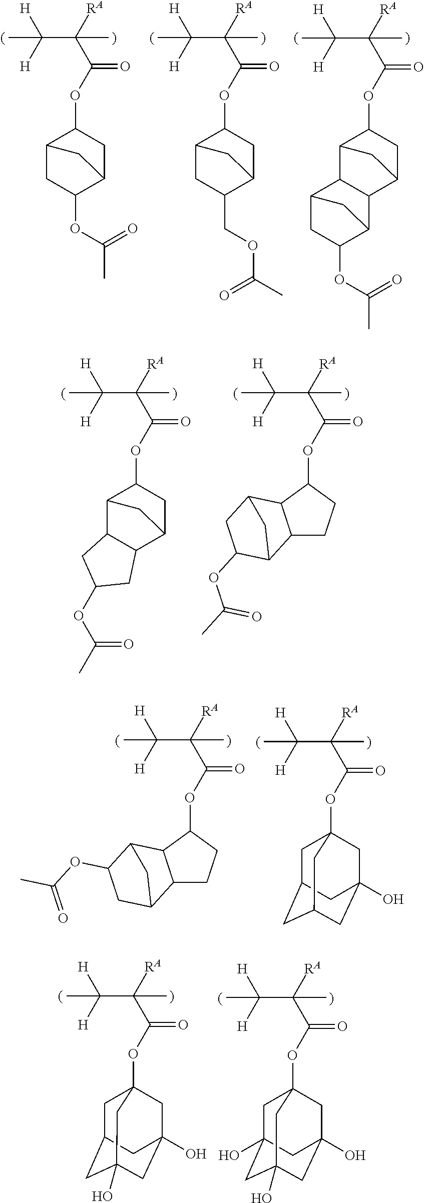

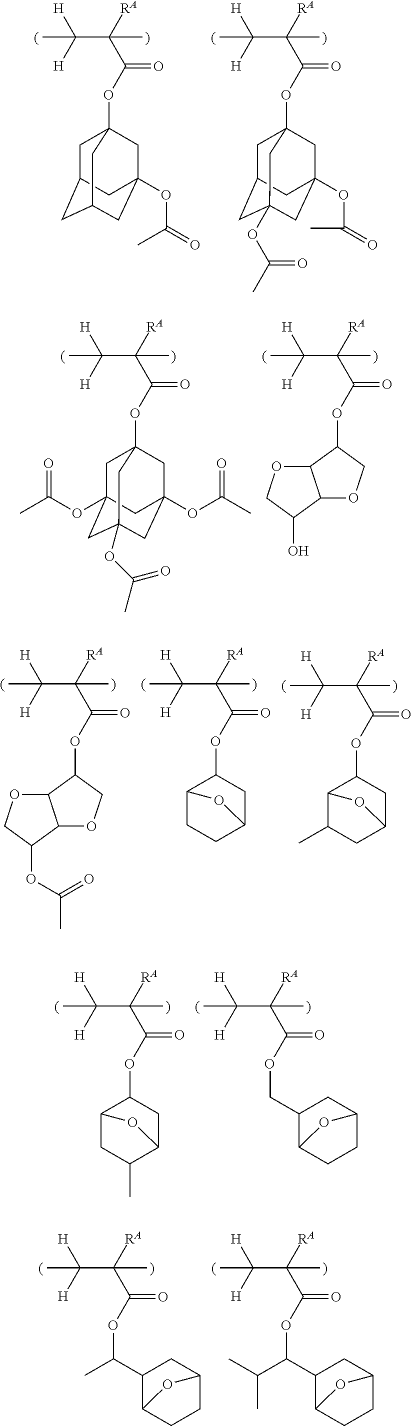

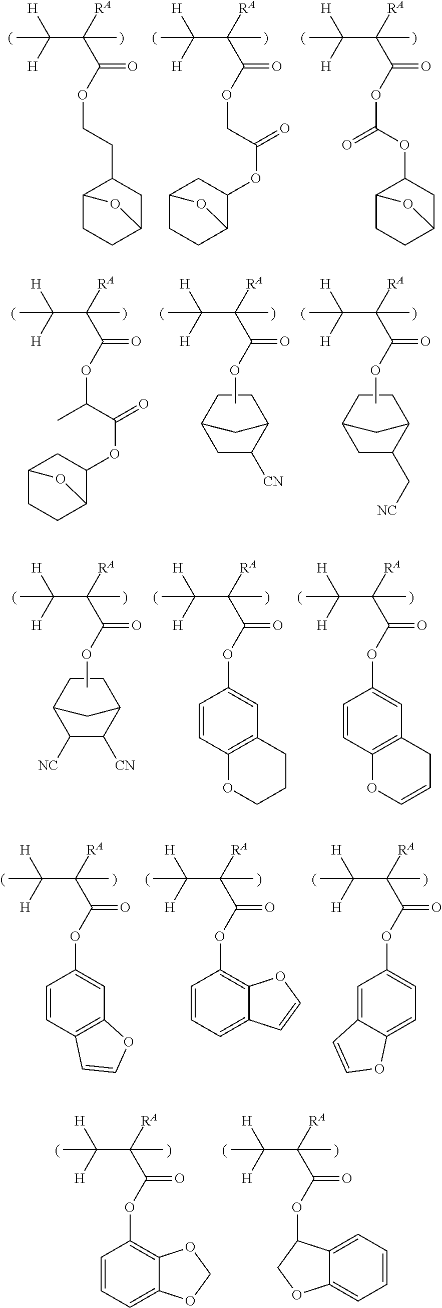

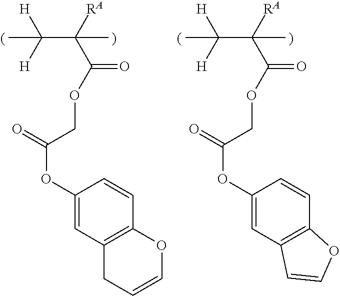

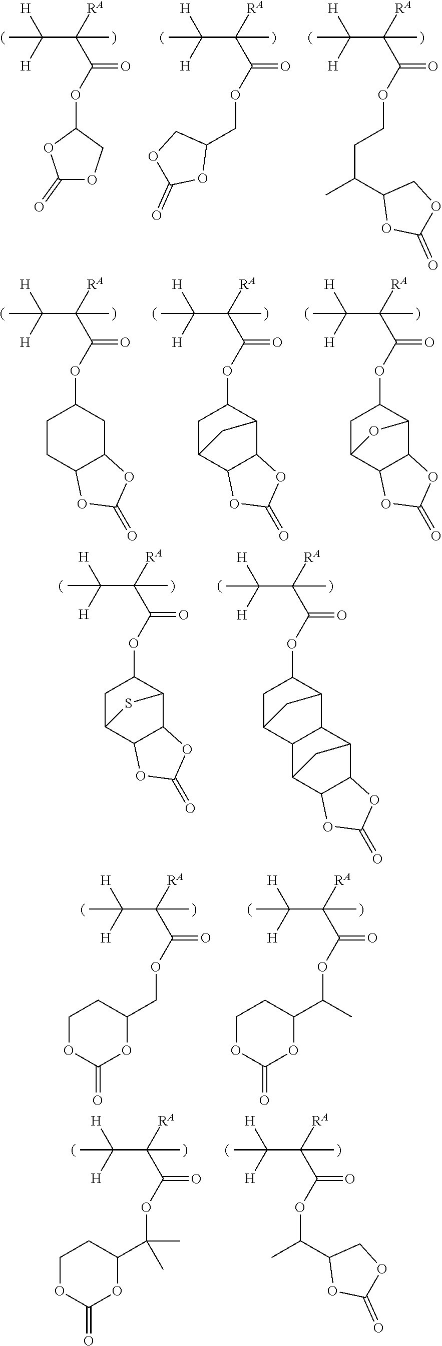

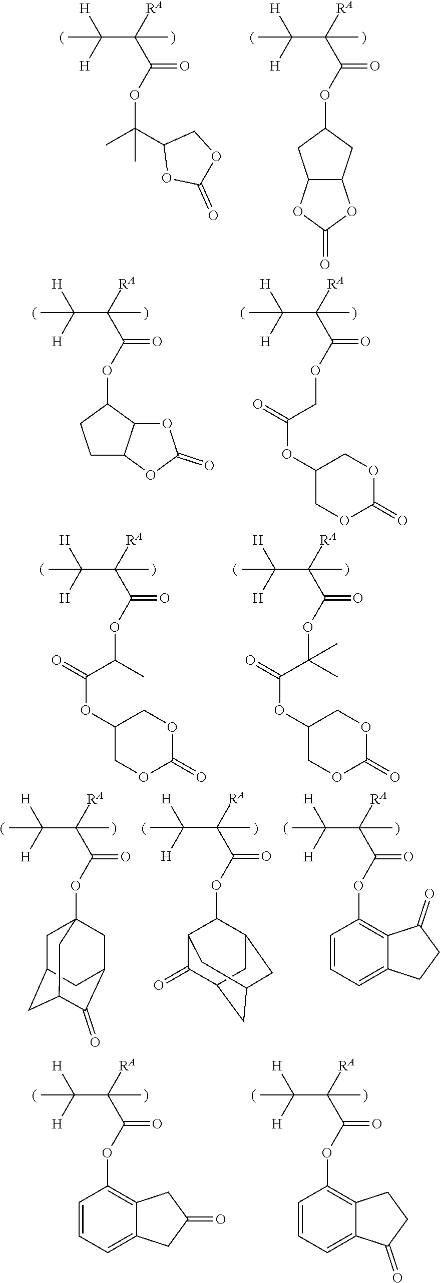

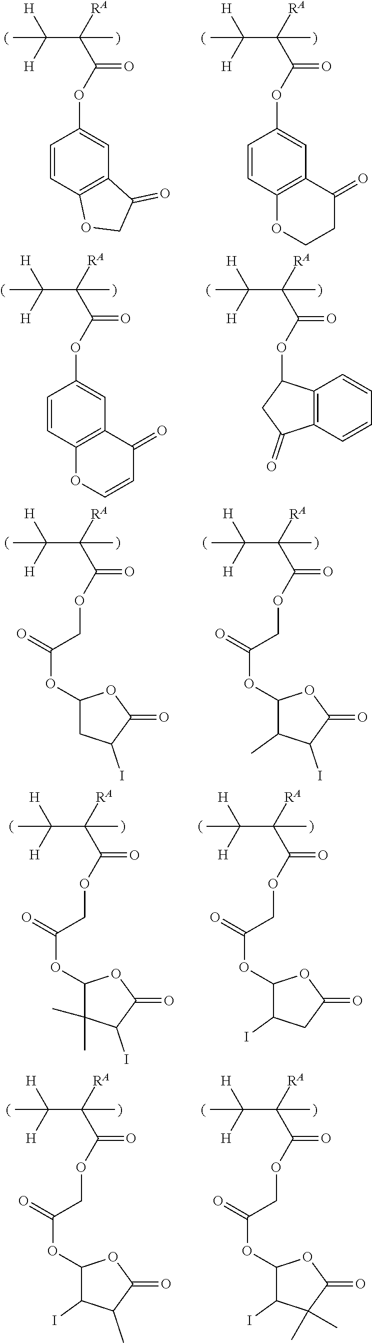

Of the repeat units (d), those units having a lactone ring as the polar group are preferred in the case of ArF lithography, and those units having a phenol site are preferred in the case of KrF, LB or EUV lithography.

The polymer may further comprise repeat units (e) of a structure having a hydroxy group protected with an acid labile group. The repeat unit (e) is not particularly limited as long as the unit includes one or more structures having a hydroxy group protected with a protective group such that the protective group is decomposed to generate a hydroxy group under the action of acid. Repeat units having the formula (e1) are preferred.

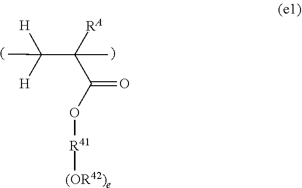

In formula (e1), R^{A} is hydrogen, fluorine, methyl or trifluoromethyl. R⁴¹ is a (e+1)-valent hydrocarbon group which may contain a heteroatom. R⁴² is an acid labile group, and e is an integer of 1 to 4.

In formula (e1), the acid labile group R⁴² is deprotected under the action of acid so that a hydroxy group is generated. Although the structure of R⁴² is not particularly limited, preferred are an acetal structure, ketal structure, alkoxycarbonyl group, and alkoxymethyl group having the following formula (e2):

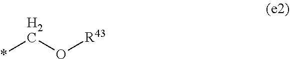

wherein R⁴³ is a C₁-C₁₅ hydrocarbyl group. The alkoxymethyl group having formula (e2) is more preferred.

Illustrative examples of the acid labile group R⁴², the alkoxymethyl group having formula (e2), and the repeat units (e) are as exemplified for the repeat units (d) in JP-A 2020-111564 (US 20200223796).

In addition to the foregoing units, the polymer may further comprise repeat units (f) derived from indene, benzofuran, benzothiophene, acenaphthylene, chromone, coumarin, and norbornadiene, or derivatives thereof. Examples of the monomer from which repeat units (f) are derived are shown below, but not limited thereto.

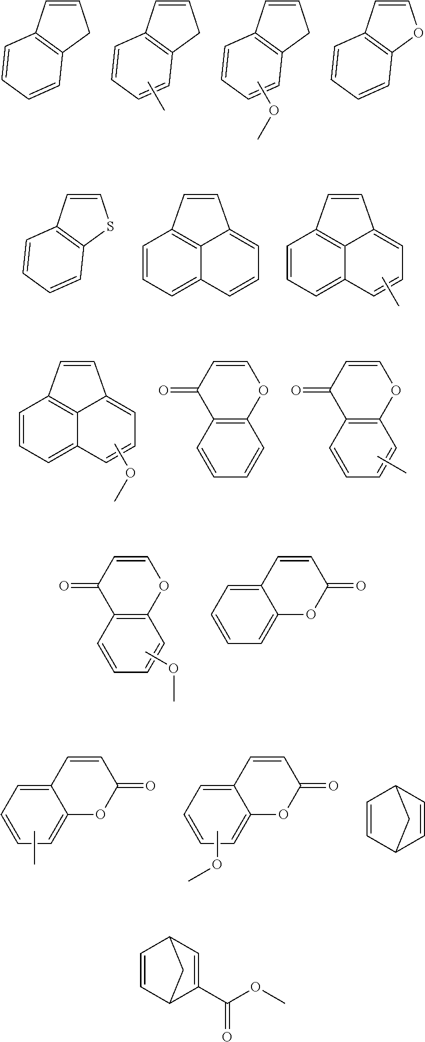

Furthermore, the polymer may comprise repeat units (g) derived from styrene, indane, vinylpyridine, vinylcarbazole, or derivatives thereof.

In the polymer, a fraction of units (a), (b1), (b2), (b3), (c), (d), (e), (f), and (g) is: preferably 0<a≤0.5, 0≤b1≤0.8, 0≤b2≤0.8, 0≤b3≤0.8, 0≤c≤0.8, 0≤d≤0.5, 0≤e≤0.3, 0≤f≤0.3, and 0≤g≤0.3; more preferably 0<a≤0.4, 0≤b1≤0.7, 0≤b2≤0.7, 0≤b3≤0.7, 0≤c≤0.7, 0≤d≤0.4, 0≤e≤0.2, 0≤f≤0.2, and 0≤g≤0.2, with the proviso: a+b1+b2+b3+c+d+e+f+g≤1.

The polymer should preferably have a weight average molecular weight (Mw) in the range of 1,000 to 500,000, and more preferably 3,000 to 100,000. A Mw in the range ensures satisfactory etch resistance and eliminates the risk of resolution being lowered due to a failure to acquire a difference in dissolution rate before and after exposure. It is noted that Mw is as measured by GPC versus polystyrene standards using tetrahydrofuran (THF) or N,N-dimethylformamide (DMF) solvent.

Since the influence of dispersity (Mw/Mn) becomes stronger as the pattern rule becomes finer, the polymer should preferably have a narrow dispersity (Mw/Mn) of 1.0 to 2.0 in order to provide a resist composition suitable for micropatterning to a small feature size. A Mw/Mn in the range indicates smaller amounts of lower and higher molecular weight fractions and eliminates the risk of leaving foreign particles on the pattern or degrading the pattern profile after exposure and development.

The polymer may be synthesized by any desired methods, for example, by dissolving one or more monomers selected from the monomers corresponding to the foregoing repeat units in an organic solvent, adding a radical polymerization initiator thereto, and heating for polymerization. Examples of the organic solvent which can be used for polymerization include toluene, benzene, tetrahydrofuran (THF), diethyl ether, dioxane, cyclohexane, cyclopentane, methyl ethyl ketone (MEK), PGMEA, and GBL. Examples of the polymerization initiator used herein include 2,2′-azobisisobutyronitrile (AIBN), 2,2′-azobis(2,4-dimethylvaleronitrile), dimethyl 2,2-azobis(2-methylpropionate), 1,1′-azobis(1-acetoxy-1-phenylethane), benzoyl peroxide, and lauroyl peroxide. The amount of the initiator added is preferably 0.01 to 25 mol % based on the total of monomers. The reaction temperature is preferably 50 to 150° C., more preferably 60 to 100° C. The reaction time is preferably 2 to 24 hours, a time of 2 to 12 hours being more preferred in view of production efficiency.

The polymerization initiator may be added to the monomer solution, which is fed to the reactor. Alternatively, a solution of the polymerization initiator is prepared separately from the monomer solution, and the monomer and initiator solutions are independently fed to the reactor. Since there is a possibility that the initiator generates a radical in the standby time, by which polymerization reaction takes place to form a ultrahigh molecular weight compound, it is preferred from the standpoint of quality control that the monomer solution and the initiator solution be independently prepared and added dropwise. The acid labile group that has been incorporated in the monomer may be kept as such, or the polymerization may be followed by protection or partial protection. Any of well-known chain transfer agents such as dodecylmercaptan and 2-mercaptoethanol may be used for the purpose of adjusting molecular weight. An appropriate amount of the chain transfer agent is 0.01 to 20 mol % based on the total of monomers to be polymerized.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, an alternative method is possible. Specifically, acetoxystyrene or acetoxyvinylnaphthalene is used instead of hydroxystyrene or hydroxyvinylnaphthalene, and after polymerization, the acetoxy group is deprotected by alkaline hydrolysis, for thereby converting the polymer product to hydroxystyrene or hydroxyvinylnaphthalene. For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The amounts of monomers in the monomer solution may be determined appropriate so as to provide the preferred fractions of repeat units as mentioned above.

It is described how to use the polymer obtained by the above preparation method. The reaction solution resulting from polymerization reaction may be used as the final product. Alternatively, the polymer may be recovered in powder form through a purifying step such as re-precipitation step of adding the reaction solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in JP-A 2008-111103, paragraphs [0144]-[0145](U.S. Pat. No. 7,537,880). Exemplary solvents include ketones such as cyclohexanone and methyl-2-n-pentyl ketone; alcohols such as 3-methoxybutanol, 3-methyl-3-methoxybutanol, 1-methoxy-2-propanol, 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; alcohols such as diacetone alcohol (DAA); and high-boiling alcohols such as diethylene glycol, propylene glycol, glycerol, 1,4-butanediol, and 1,3-butanediol, which may be used alone or in admixture.

The polymer solution preferably has a polymer concentration of 0.01 to 30% by weight, more preferably 0.1 to 20% by weight.

Prior to use, the reaction solution or polymer solution is preferably filtered through a filter. Filtration is effective for consistent quality because foreign particles and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of up to 100 nm, more preferably up to 20 nm. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

### [Chemically Amplified Resist Composition]

A further embodiment of the invention is a chemically amplified resist composition comprising (A) a base polymer containing the polymer defined above.

### (A) Base Polymer

The polymer defined above may be used alone or as a mixture of two or more polymers which are different in compositional ratio, Mw and/or Mw/Mn. In addition to the polymer, the base polymer (A) may contain a hydrogenated product of ring-opening metathesis polymerization (ROMP) polymer, which is described in JP-A 2003-066612.

### (B) Quencher

The resist composition may further comprise (B) a quencher. As used herein, the “quencher” refers to a compound capable of trapping the acid generated by the PAG to prevent the acid from diffusing into the unexposed region of resist film, for forming the desired pattern.

Preferred examples of the quencher include onium salts having the formulae (1) and (2).

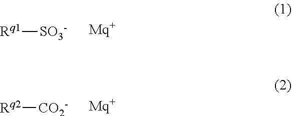

In formula (1), R^{q1} is hydrogen or a C₁-C₄₀ hydrocarbyl group which may contain a heteroatom, exclusive of the group wherein hydrogen bonded to the carbon atom at α-position relative to the sulfo group is substituted by fluorine or fluoroalkyl. In formula (2), R^{q2} is hydrogen or a C₁-C₄₀ hydrocarbyl group which may contain a heteroatom.

Examples of the C₁-C₄₀ hydrocarbyl group R^{q1} include C₁-C₄₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₄₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decyl, and adamantyl; C₆-C₄₀ aryl groups such as phenyl, naphthyl and anthracenyl. In the hydrocarbyl group, some or all hydrogen may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, carbonyl moiety, ether bond, ester bond, sulfonic ester bond, carbonate bond, lactone ring, sultone ring, carboxylic anhydride (—C(═O)—O—C(═O)—), or haloalkyl moiety.

Examples of the hydrocarbyl group R^{q2} include those exemplified above for R^{q1} and fluorinated saturated hydrocarbyl groups, for example, fluorinated alkyl groups such as trifluoromethyl and trifluoroethyl, and fluorinated aryl groups such as pentafluorophenyl and 4-trifluoromethylphenyl.

Examples of the anion in the onium salt having formula (1) are shown below, but not limited thereto.

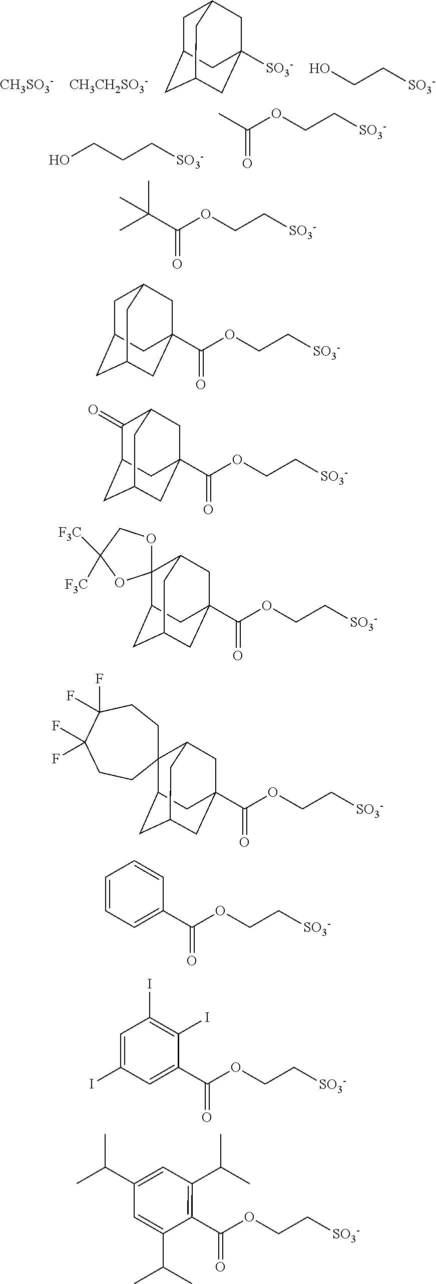

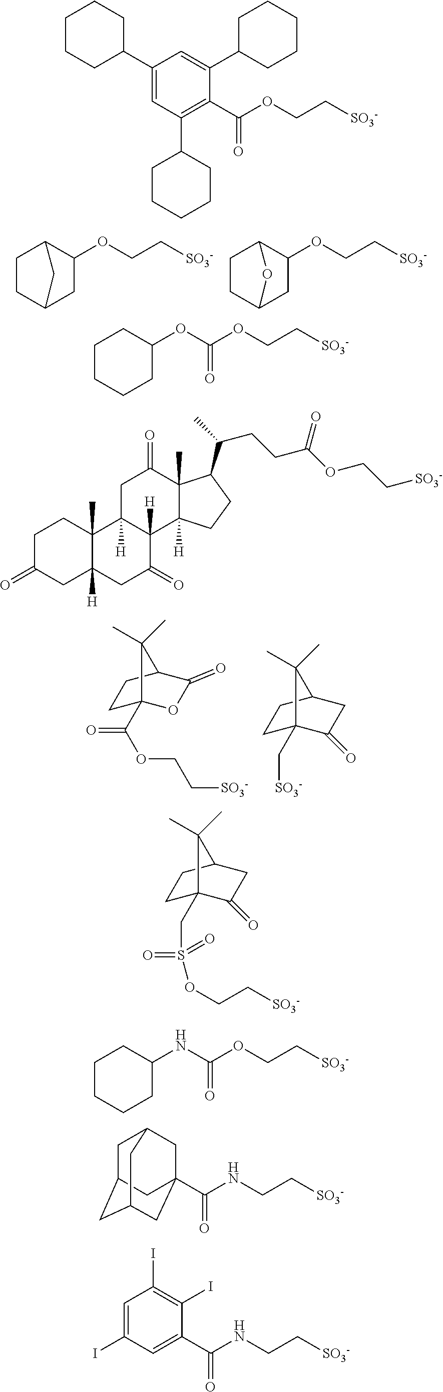

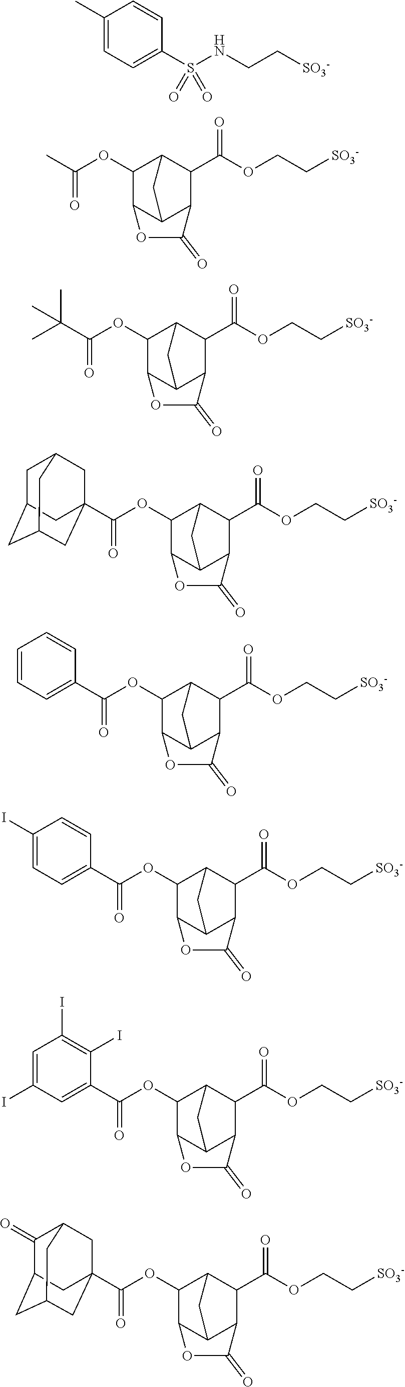

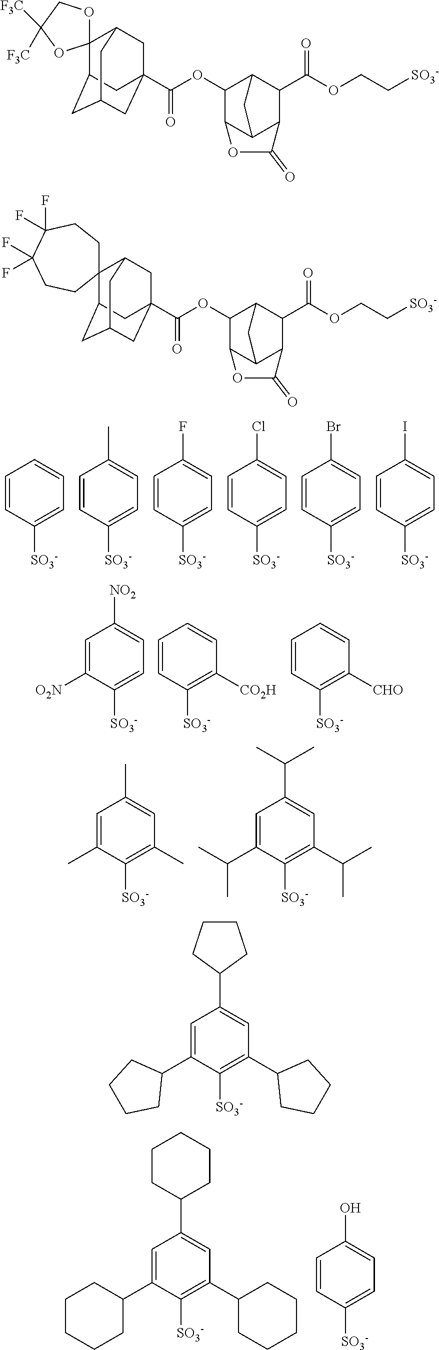

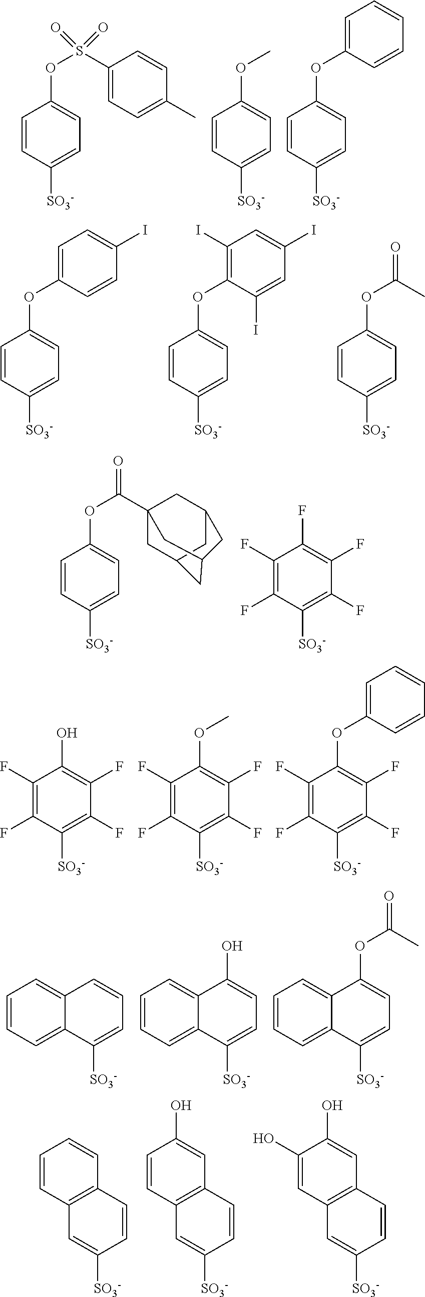

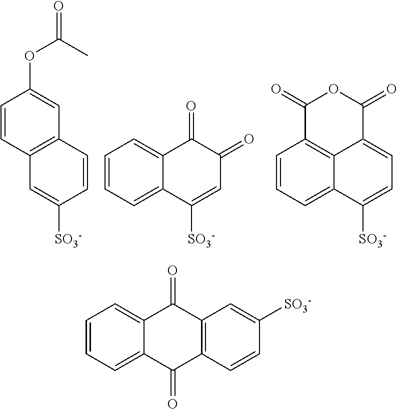

Examples of the anion in the onium salt having formula (2) are shown below, but not limited thereto.

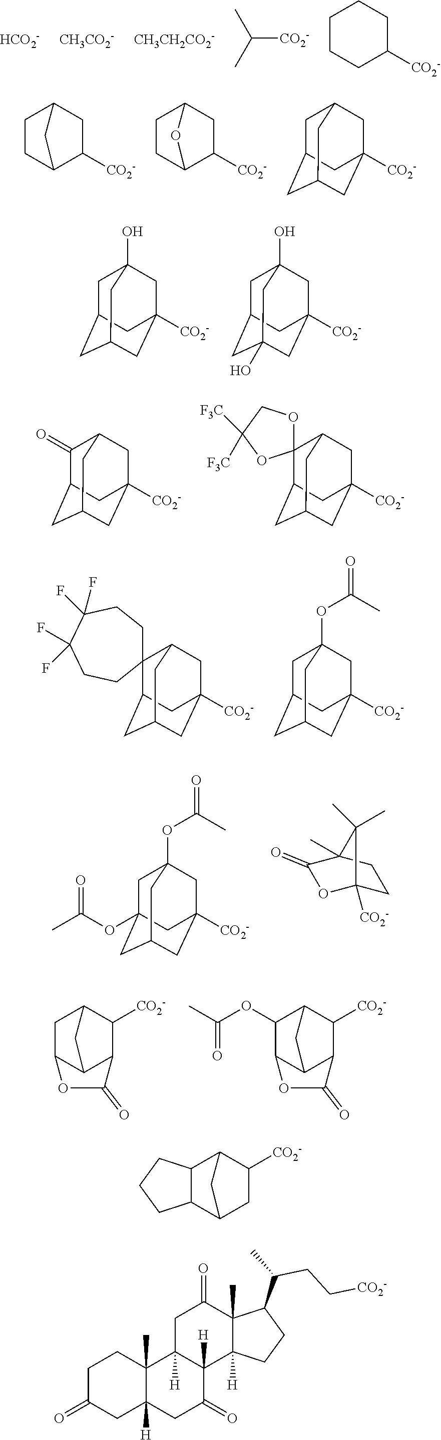

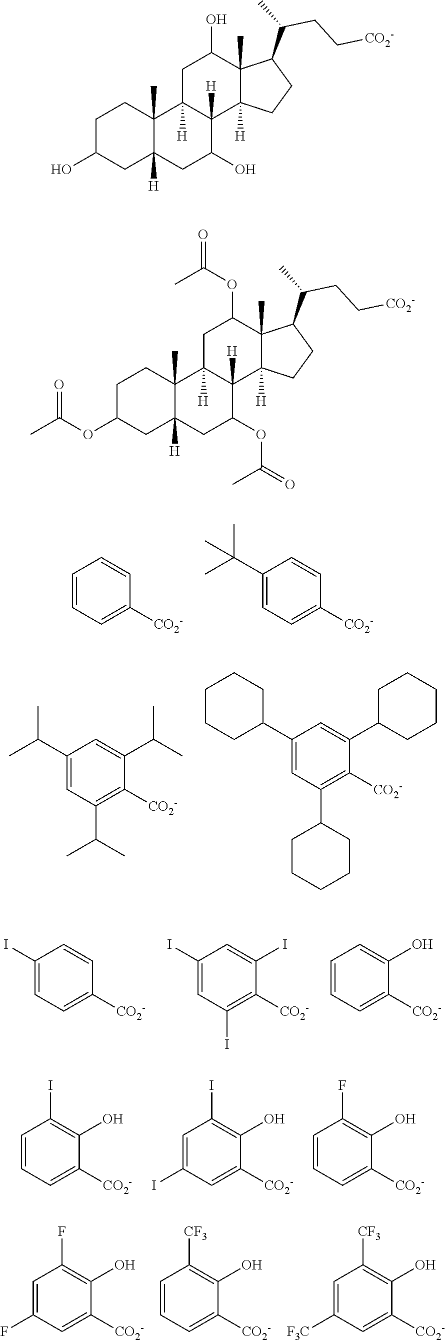

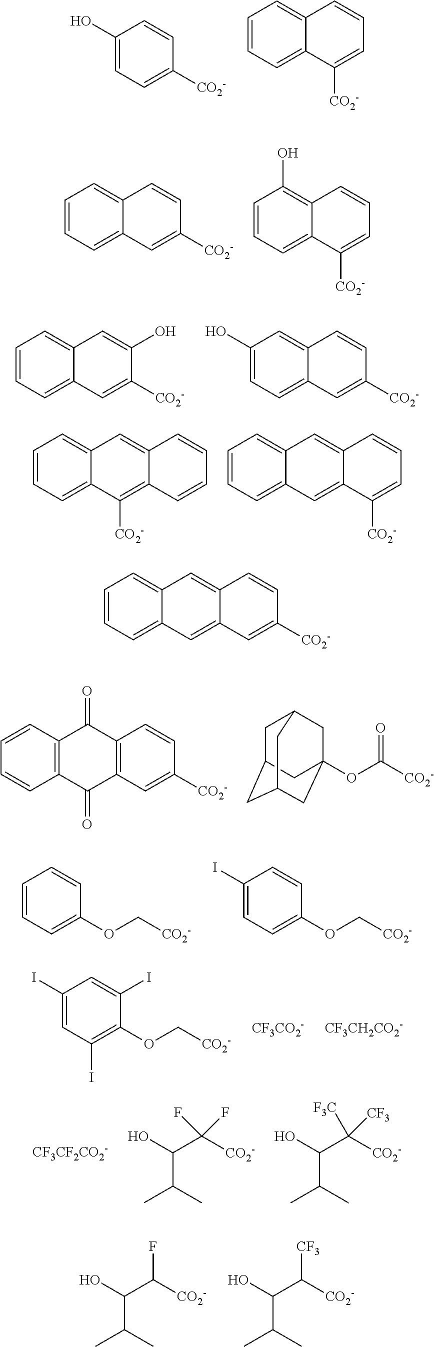

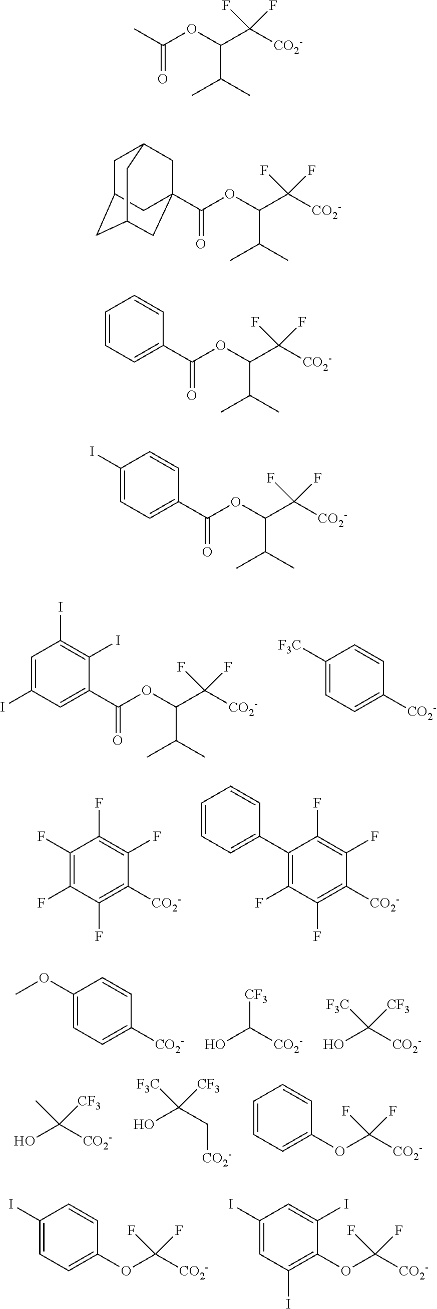

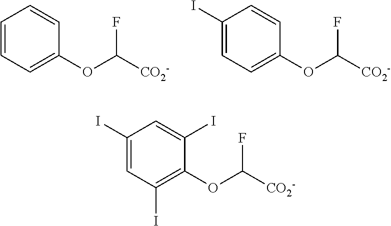

In formulae (1) and (2), Mq⁺ is an onium cation. The onium cation is preferably selected from sulfonium cations having the formula (cation-1), iodonium cations having the formula (cation-2), and ammonium cations having the formula (cation-3).

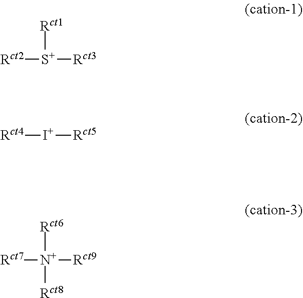

In formulae (cation-1) and (cation-2), R^{ct1} to R^{ct5} are each independently halogen or a C₁-C₃₀ hydrocarbyl group which may contain a heteroatom.

Suitable halogen atoms include fluorine, chlorine, bromine, and iodine.

The hydrocarbyl group may be saturated or unsaturated and straight, branched or cyclic. Examples thereof include C₁-C₃₀ alkyl groups such as methyl, ethyl, n-propyl, isopropyl, n-butyl, isobutyl, sec-butyl, and tert-butyl; C₃-C₃₀ cyclic saturated hydrocarbyl groups such as cyclopropyl, cyclopentyl, cyclohexyl, cylopropylmethyl, 4-methylcyclohexyl, cyclohexylmethyl, norbornyl, and adamantyl; C₂-C₃₀ alkenyl groups such as vinyl, allyl, butenyl, and hexenyl; C₃-C₃₀ cyclic unsaturated hydrocarbyl groups such as cyclohexenyl; C₆-C₃₀ aryl groups such as phenyl, naphthyl, and thienyl; C₇-C₃₀ aralkyl groups such as benzyl, 1-phenylethyl, and 2-phenylethyl, and combinations thereof. Of these, aryl groups are preferred. In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy moiety, fluorine, chlorine, bromine, iodine, cyano moiety, nitro moiety, carbonyl moiety, ether bond, ester bond, sulfonic ester bond, carbonate bond, lactone ring, sultone ring, carboxylic anhydride (—C(═O)—O—C(═O)—) or haloalkyl moiety.

Also, R^{ct1} and R^{ct2} may bond together to form a ring with the sulfur atom to which they are attached. Examples of the ring are shown below.

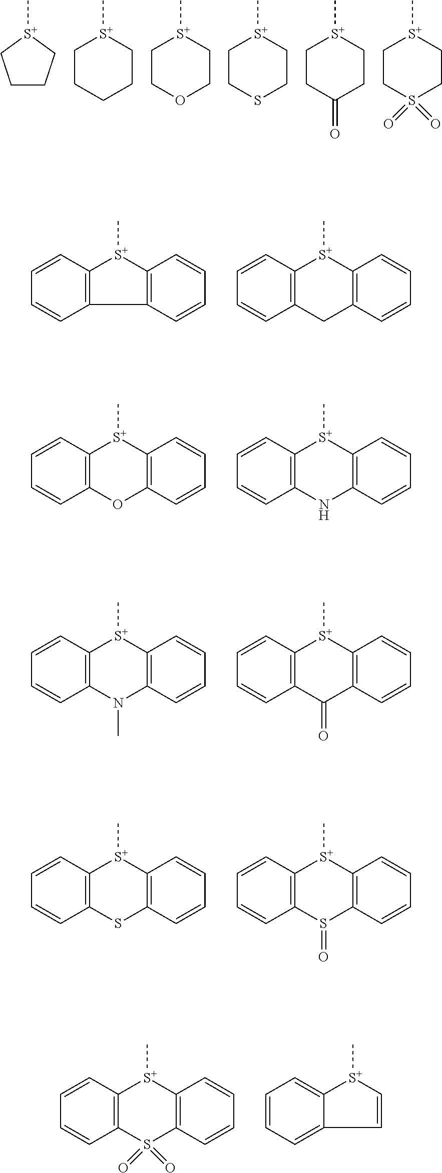

Herein the broken line designates a point of attachment to R^{ct3}.

Examples of the sulfonium cation having formula (cation-1) are shown below, but not limited thereto.

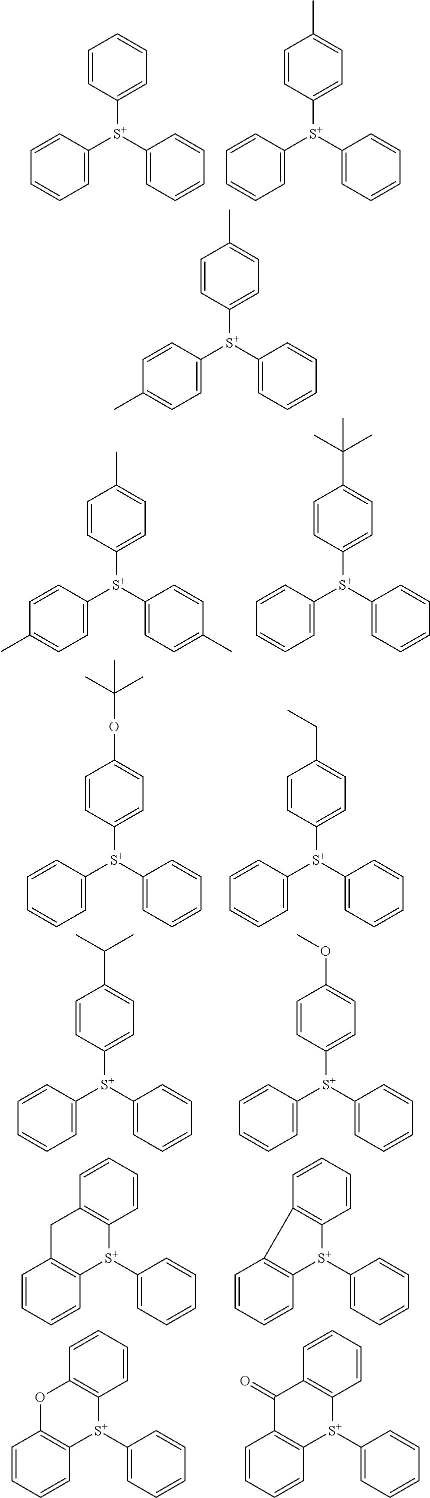

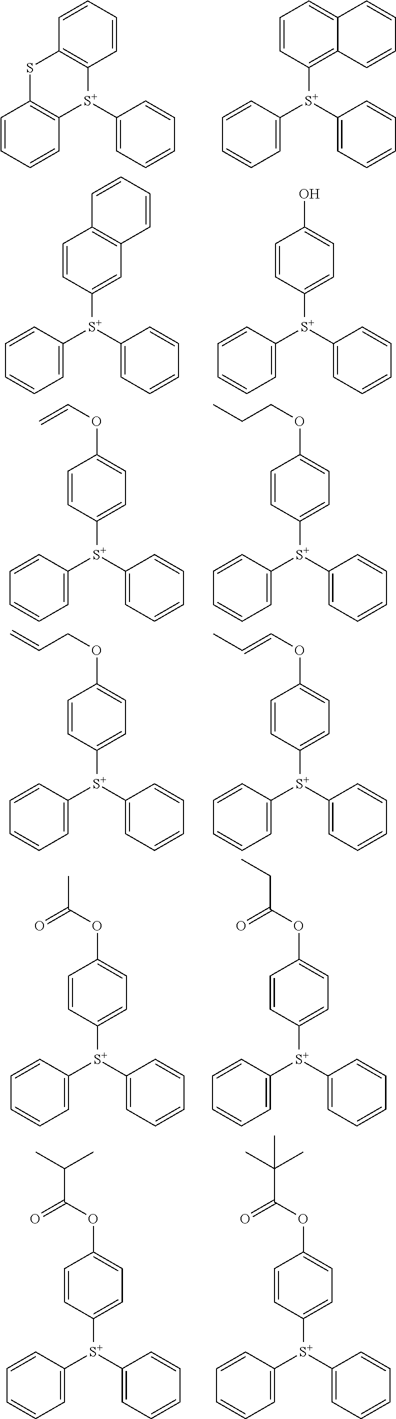

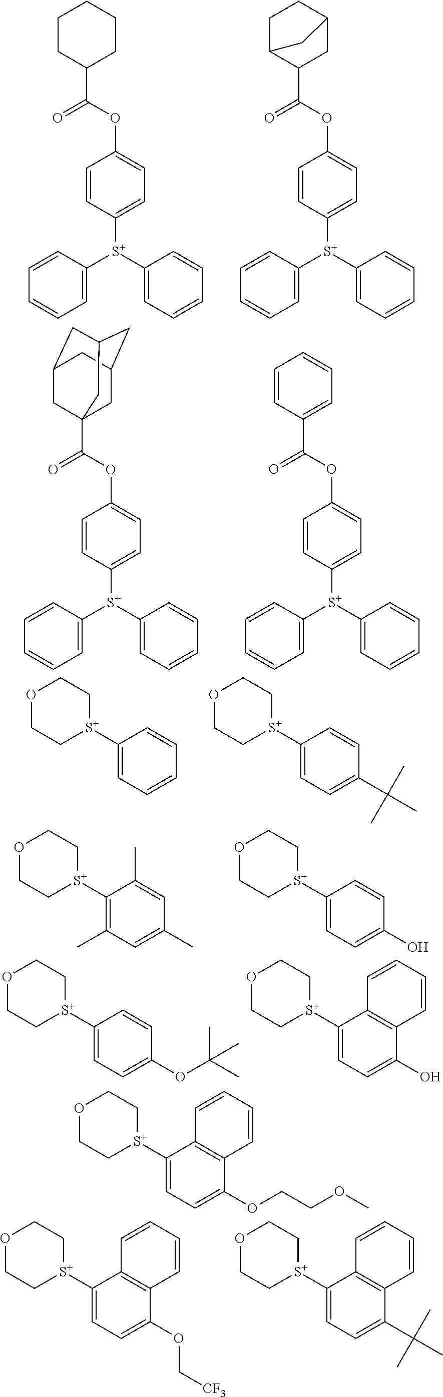

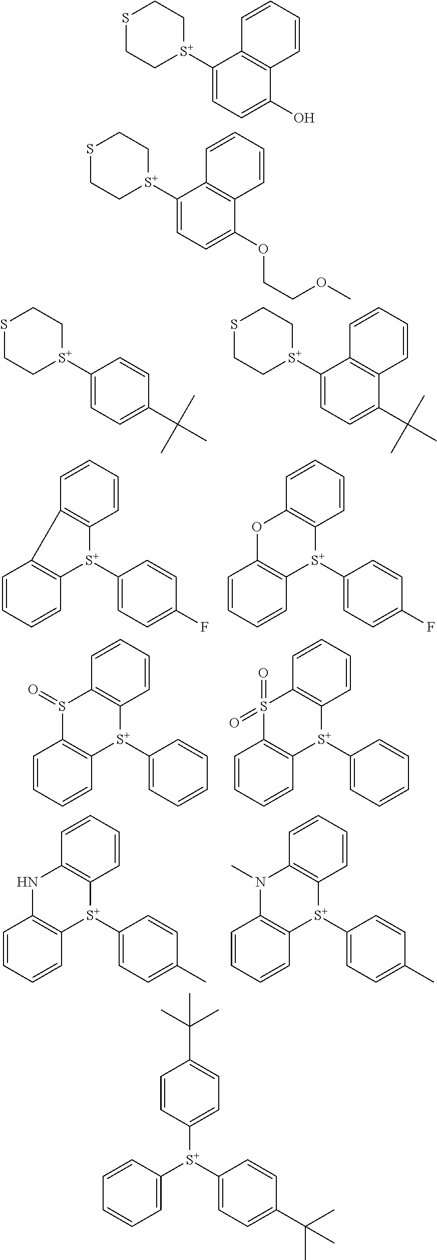

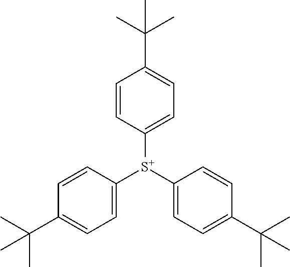 [SMILES: CC(C)(C)c1ccc([S+](c2ccc(C(C)(C)C)cc2)c2ccc(C(C)(C)C)cc2)cc1]

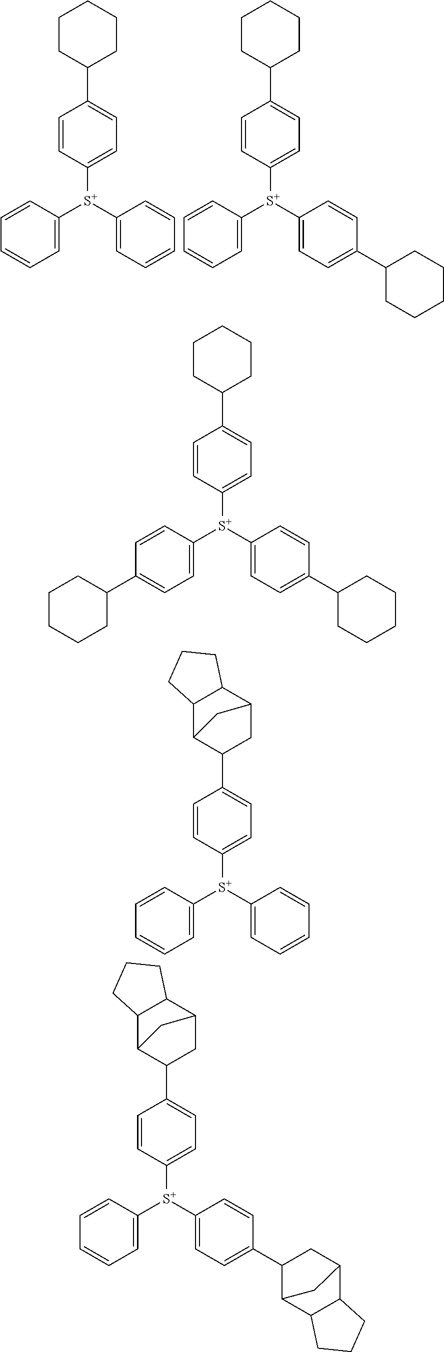 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1 ; c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1]

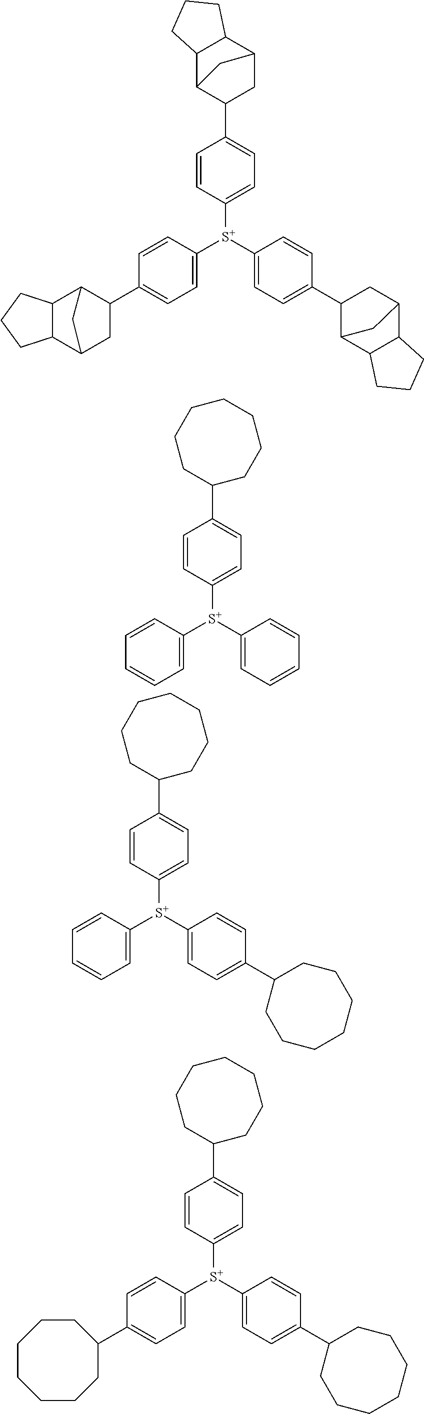 [SMILES: c1cc([S+](c2ccc(C3CC4CC3C3CCCC43)cc2)c2ccc(C3CC4CC3C3CCCC43)cc2)ccc1C1CC2CC1C1CCCC21 ; c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1]

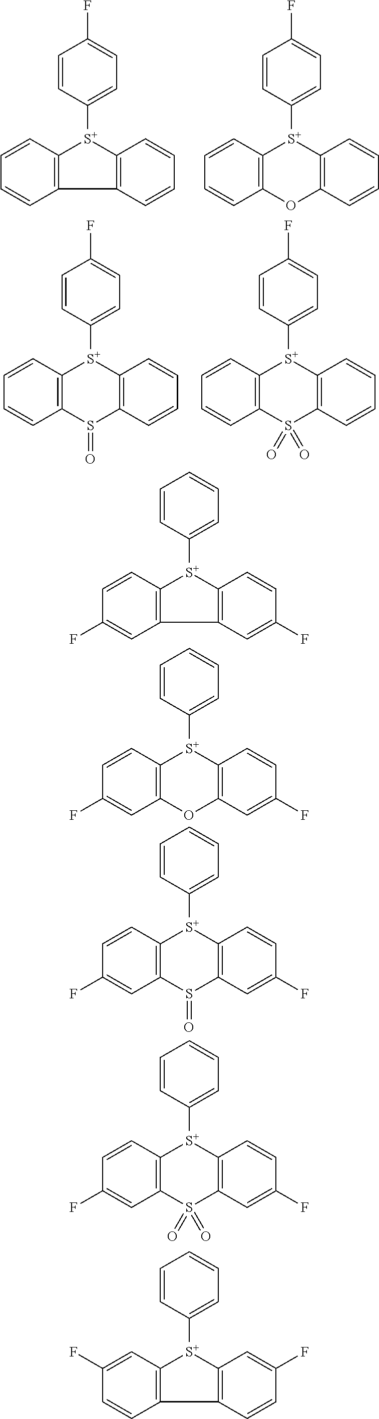

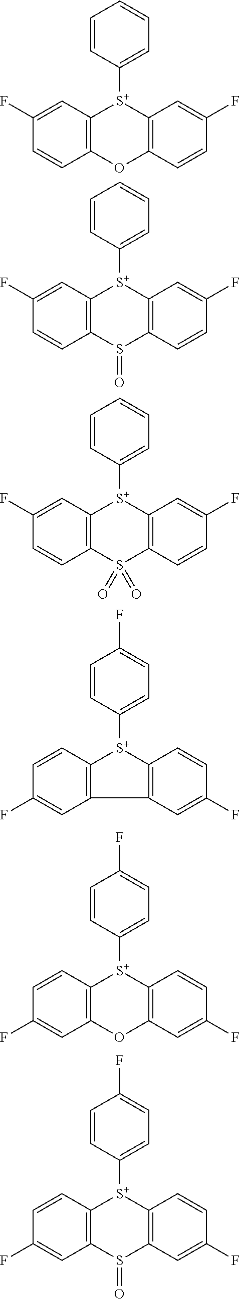 [SMILES: Fc1ccc2c(c1)[S+](c1ccccc1)c1cc(F)ccc1O2 ; O=S1c2ccc(F)cc2[S+](c2ccccc2)c2cc(F)ccc21 ; O=S1(=O)c2ccc(F)cc2[S+](c2ccccc2)c2cc(F)ccc21 ; Fc1ccc(-[s+]2c3ccc(F)cc3c3cc(F)ccc32)cc1 ; Fc1ccc([S+]2c3ccc(F)cc3Oc3cc(F)ccc32)cc1 ; O=S1c2cc(F)ccc2[S+](c2ccc(F)cc2)c2ccc(F)cc21]

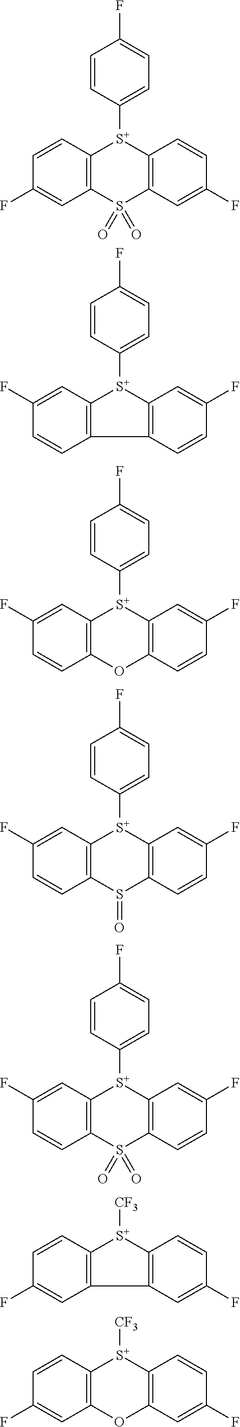

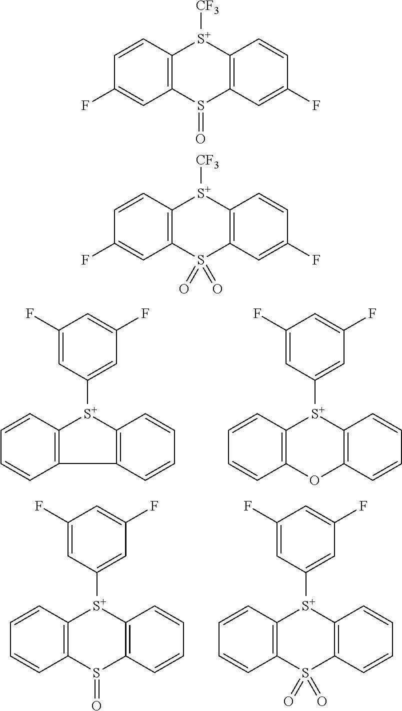 [SMILES: O=S1c2cc(F)ccc2[S+](C(F)(F)F)c2ccc(F)cc21 ; O=S1(=O)c2cc(F)ccc2[S+](C(F)(F)F)c2ccc(F)cc21 ; Fc1cc(F)cc(-[s+]2c3ccccc3c3ccccc32)c1 ; Fc1cc(F)cc([S+]2c3ccccc3Oc3ccccc32)c1 ; O=S1c2ccccc2[S+](c2cc(F)cc(F)c2)c2ccccc21 ; O=S1(=O)c2ccccc2[S+](c2cc(F)cc(F)c2)c2ccccc21]

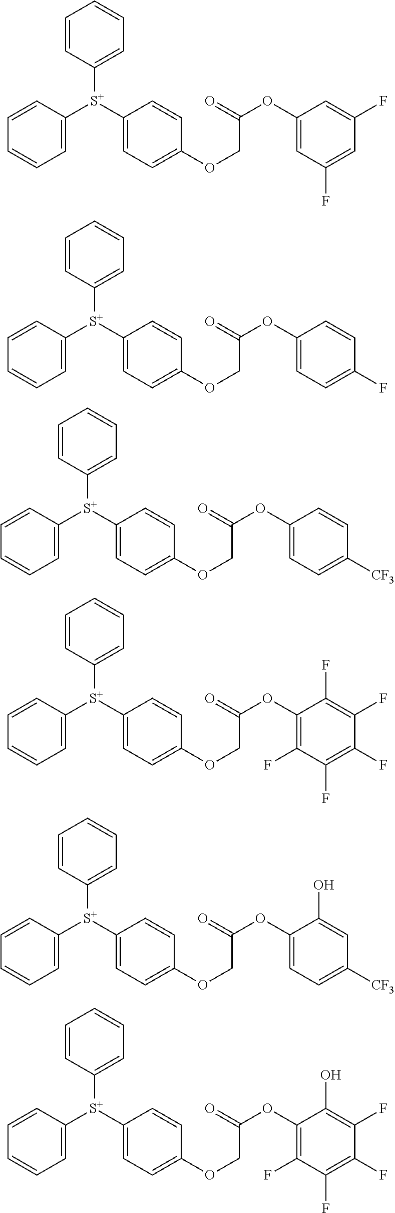 [SMILES: O=C(COc1ccc([S+](c2ccccc2)c2ccccc2)cc1)Oc1cc(F)cc(F)c1 ; O=C(COc1ccc([S+](c2ccccc2)c2ccccc2)cc1)Oc1ccc(F)cc1 ; O=C(COc1ccc([S+](c2ccccc2)c2ccccc2)cc1)Oc1ccc(C(F)(F)F)cc1 ; O=C(COc1ccc([S+](c2ccccc2)c2ccccc2)cc1)Oc1c(F)c(F)c(F)c(F)c1F ; O=C(COc1ccc([S+](c2ccccc2)c2ccccc2)cc1)Oc1ccc(C(F)(F)F)cc1O ; O=C(COc1ccc([S+](c2ccccc2)c2ccccc2)cc1)Oc1c(O)c(F)c(F)c(F)c1F]

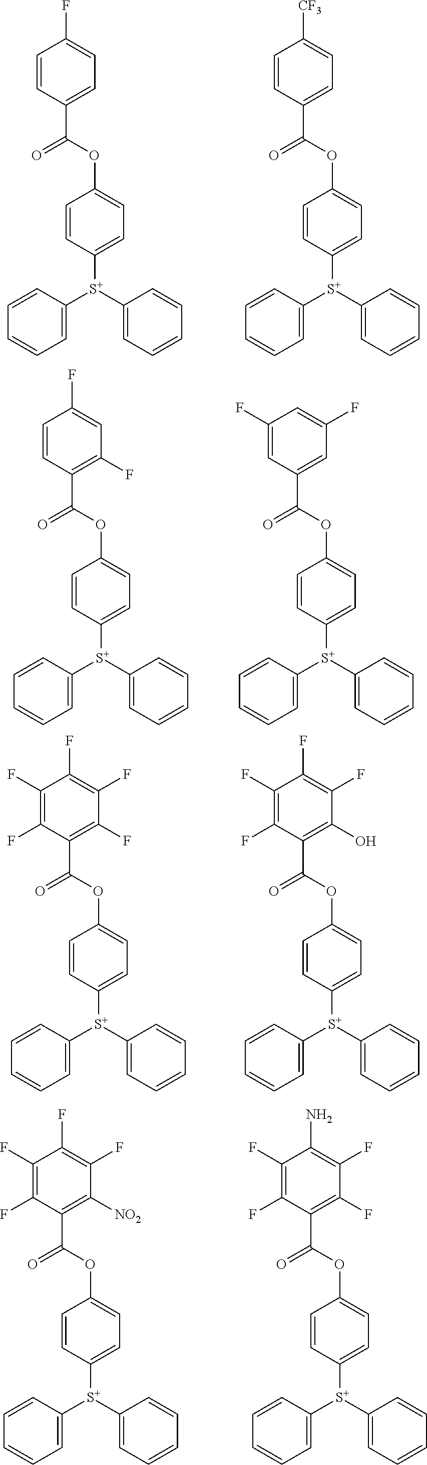

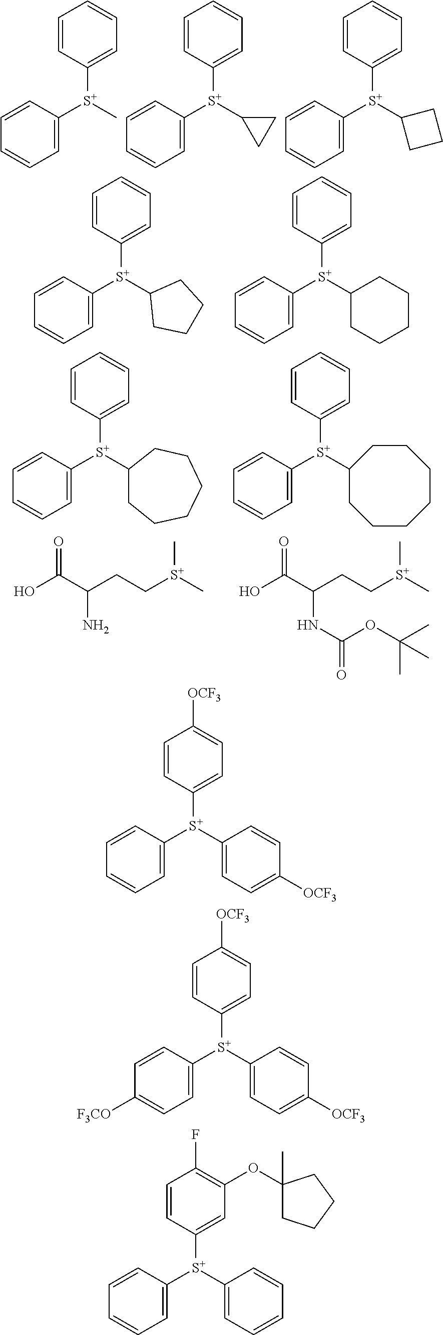

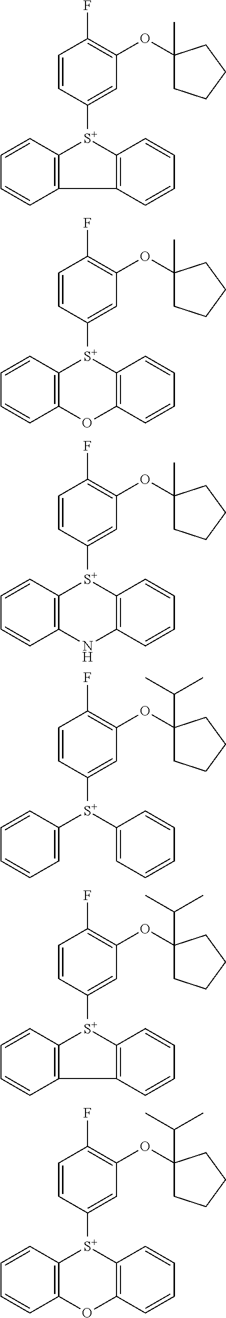 [SMILES: CC1(Oc2cc(-[s+]3c4ccccc4c4ccccc43)ccc2F)CCCC1 ; CC1(Oc2cc([S+]3c4ccccc4Oc4ccccc43)ccc2F)CCCC1 ; CC1(Oc2cc([S+]3c4ccccc4Nc4ccccc43)ccc2F)CCCC1 ; CC(C)C1(Oc2cc([S+](c3ccccc3)c3ccccc3)ccc2F)CCCC1 ; CC(C)C1(Oc2cc(-[s+]3c4ccccc4c4ccccc43)ccc2F)CCCC1 ; CC(C)C1(Oc2cc([S+]3c4ccccc4Oc4ccccc43)ccc2F)CCCC1]

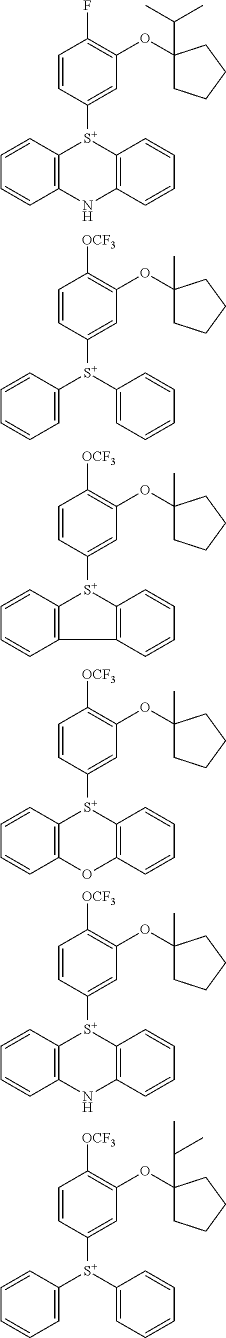 [SMILES: CC(C)C1(Oc2cc([S+]3c4ccccc4Nc4ccccc43)ccc2F)CCCC1 ; CC1(Oc2cc([S+](c3ccccc3)c3ccccc3)ccc2OC(F)(F)F)CCCC1 ; CC1(Oc2cc(-[s+]3c4ccccc4c4ccccc43)ccc2OC(F)(F)F)CCCC1 ; CC1(Oc2cc([S+]3c4ccccc4Oc4ccccc43)ccc2OC(F)(F)F)CCCC1 ; CC1(Oc2cc([S+]3c4ccccc4Nc4ccccc43)ccc2OC(F)(F)F)CCCC1 ; CC(C)C1(Oc2cc([S+](c3ccccc3)c3ccccc3)ccc2OC(F)(F)F)CCCC1]

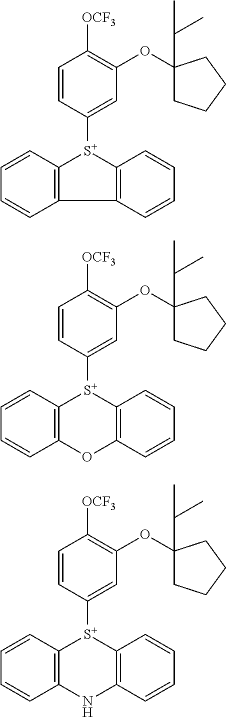 [SMILES: CC(C)C1(Oc2cc(-[s+]3c4ccccc4c4ccccc43)ccc2OC(F)(F)F)CCCC1 ; CC(C)C1(Oc2cc([S+]3c4ccccc4Oc4ccccc43)ccc2OC(F)(F)F)CCCC1 ; CC(C)C1(Oc2cc([S+]3c4ccccc4Nc4ccccc43)ccc2OC(F)(F)F)CCCC1]

Examples of the iodonium cation having formula (cation-2) are shown below, but not limited hereto.

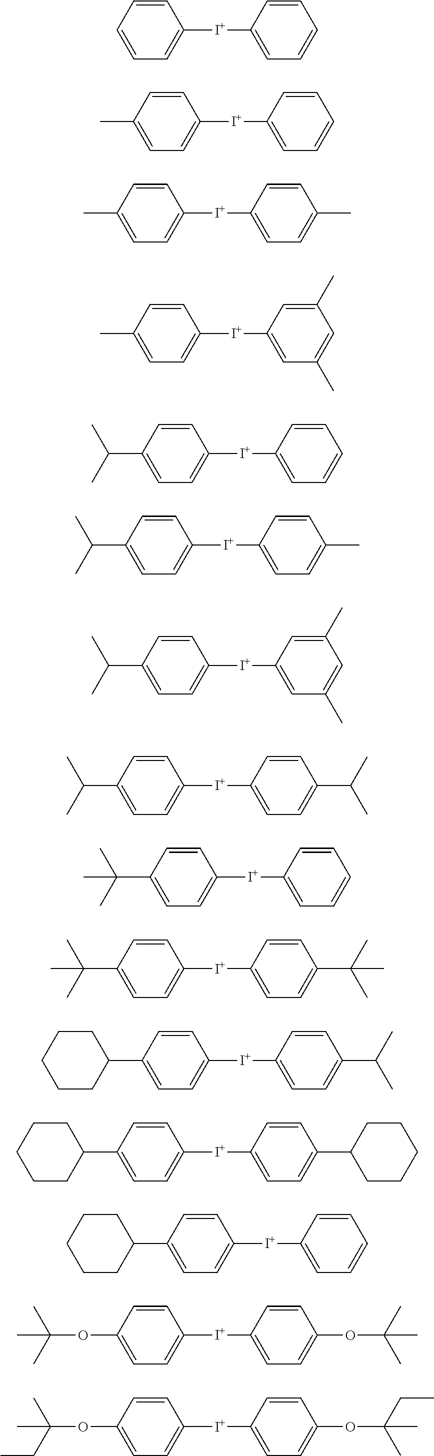

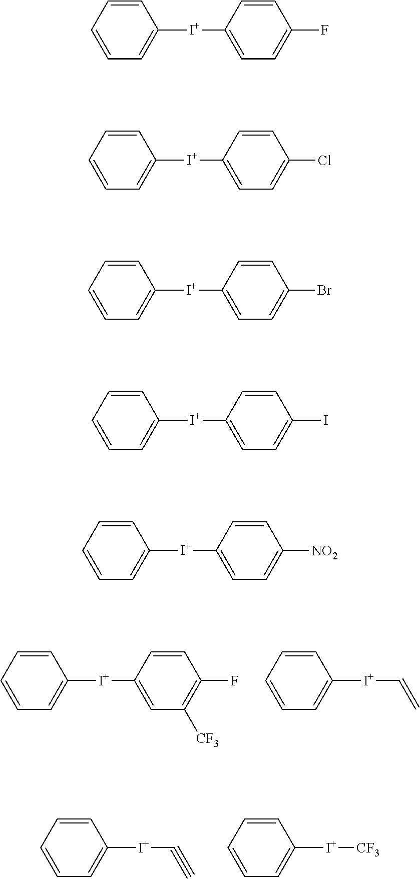

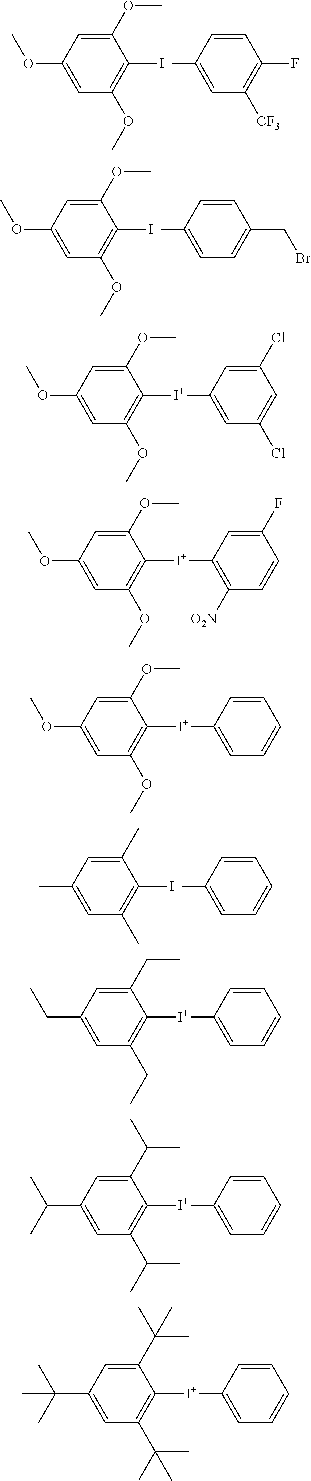

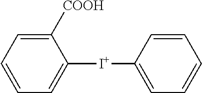 [SMILES: O=C(O)c1ccccc1[I+]c1ccccc1]

In formula (cation-3), R^{ct6} to R^{ct9} are each independently a C₁-C₄₀ hydrocarbyl group which may contain a heteroatom. A pair of R^{ct6} and R^{ct7} may bond together to form a ring with the nitrogen atom to which they are attached. Examples of the hydrocarbyl group are as exemplified above for the hydrocarbyl groups R^{ct1} to R^{ct5} in formulae (cation-1) and (cation-2).

Examples of the ammonium cation having formula (cation-3) are shown below, but not limited thereto.

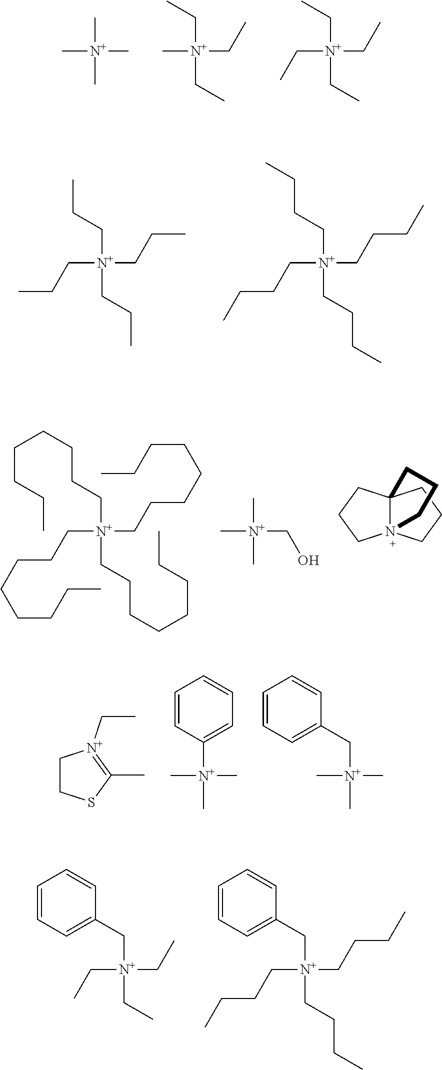

Examples of the onium salt having formula (1) or (2) include arbitrary combinations of anions with cations, both as exemplified above. These onium salts may be readily synthesized by ion exchange reaction according to any well-known organic chemistry technique. For the ion exchange reaction, reference may be made to JP-A 2007-145797, for example.

The onium salt having formula (1) or (2) functions as a quencher in the resist composition because the counter anion of the onium salt is a conjugated base of a weak acid. As used herein, the weak acid indicates an acidity insufficient to deprotect an acid labile group from an acid labile group-containing unit in the base polymer. The onium salt having formula (1) or (2) functions as a quencher when used in combination with an onium salt type PAG having a conjugated base of a strong acid (typically α-fluorinated sulfonic acid) as the counter anion. In a system using a mixture of an onium salt capable of generating a strong acid (e.g., α-fluorinated sulfonic acid) and an onium salt capable of generating a weak acid (e.g., non-fluorinated sulfonic acid or carboxylic acid), if the strong acid generated from the PAG upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

Also useful as the quencher (B) are onium salts having a sulfonium cation and a phenoxide anion site in a common molecule as described in JP 6848776, onium salts having a sulfonium cation and a carboxylate anion site in a common molecule as described in JP 6583136 and JP-A 2020-200311, and onium salts having an iodonium cation and a carboxylate anion site in a common molecule as described in JP 6274755.

If a PAG capable of generating a strong acid is an onium salt, an exchange from the strong acid generated upon exposure to high-energy radiation to a weak acid as above can take place, but it rarely happens that the weak acid generated upon exposure to high-energy radiation collides with the unreacted onium salt capable of generating a strong acid to induce a salt exchange. This is because of a likelihood of an onium cation forming an ion pair with a stronger acid anion.

When the onium salt having formula (1) or (2) is used as the quencher (B), the amount of the onium salt used is preferably 0.1 to 20 parts by weight, more preferably 0.1 to 10 parts by weight per 80 parts by weight of the base polymer (A). As long as the amount of component (B) is in the range, a satisfactory resolution is available without a substantial lowering of sensitivity. The onium salt having formula (1) or (2) may be used alone or in admixture.

Nitrogen-containing compounds may also be used as the quencher (B). Suitable nitrogen-containing compounds include primary, secondary and tertiary amine compounds, specifically amine compounds having a hydroxy group, ether bond, ester bond, lactone ring, cyano group or sulfonic ester bond, as described in JP-A 2008-111103, paragraphs [0146]-[0164](U.S. Pat. No. 7,537,880), and primary or secondary amine compounds protected with a carbamate group, as described in JP 3790649. Also useful are compounds having an acid labile group within the molecule as described in JP-A 2009-109595.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-046501, for example.

When the nitrogen-containing compound is used as the quencher (B), the amount of the nitrogen-containing compound used is preferably 0.001 to 12 parts by weight, more preferably 0.01 to 8 parts by weight per 80 parts by weight of the base polymer (A). The nitrogen-containing compound may be used alone or in admixture.

### (C) Organic Solvent

The resist composition may comprise (C) an organic solvent. The organic solvent used herein is not particularly limited as long as the foregoing and other components are soluble therein. Suitable solvents include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone (GBL), and mixtures thereof.

Of the foregoing organic solvents, it is recommended to use 1-ethoxy-2-propanol, PGMEA, cyclohexanone, GBL, DAA, and mixtures thereof because the base polymer (A) is most soluble therein.

The organic solvent (C) is preferably added in an amount of 200 to 5,000 parts by weight, and more preferably 400 to 3,500 parts by weight per 80 parts by weight of the base polymer (A). The organic solvent may be used alone or in admixture.

### (D) Acid Generator

The resist composition may comprise (D) an acid generator as long as the benefits of the invention are not impaired. The acid generator is typically a compound (PAG) capable of generating an acid upon exposure to actinic ray or radiation. Although the PAG used herein may be any compound capable of generating an acid upon exposure to high-energy radiation, those compounds capable of generating a sulfonic acid, imide acid (imidic acid) or methide acid are preferred. Suitable PAGs include sulfonium salts, iodonium salts, sulfonyldiazomethane, N-sulfonyloxyimide, and oxime-O-sulfonate acid generators. Exemplary PAGs are described in JP-A 2008-111103, paragraphs [0122]-[0142](U.S. Pat. No. 7,537,880).

As the PAG used herein, sulfonium salts having the formula (3-1) and iodonium salts having the formula (3-2) are also preferred.

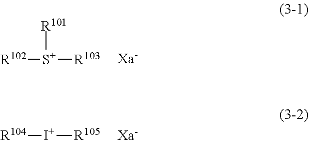

In formulae (3-1) and (3-2), R¹⁰¹ to R¹⁰⁵ are each independently halogen or a C₁-C₂₀ hydrocarbyl group which may contain a heteroatom. Examples of the halogen and hydrocarbyl group are as exemplified above for the halogen and hydrocarbyl group represented by R^{ct1} to R^{ct5} in formulae (cation-1) and (cation-2). Also included are substituted forms of the foregoing hydrocarbyl groups in which some or all of the hydrogen atoms are substituted by a moiety containing a heteroatom such as oxygen, sulfur, nitrogen or halogen, or some constituent —CH2- is replaced by a moiety containing a heteroatom such as oxygen, sulfur or nitrogen, so that the group may contain a hydroxy moiety, fluorine, chlorine, bromine, iodine, cyano moiety, nitro moiety, carbonyl moiety, ether bond, ester bond, sulfonic ester bond, carbonate moiety, lactone ring, sultone ring, carboxylic anhydride (—C(═O)—O—C(═O)—) or haloalkyl moiety. A pair of R¹⁰¹ and R¹⁰² may bond together to form a ring with the sulfur atom to which they are attached. Preferred examples of the ring are as exemplified above for the ring that R^{ct1} and R^{ct2} in formula (cation-1), taken together, form with the sulfur atom to which they are attached.

Examples of the cation in the sulfonium salt having formula (3-1) are as exemplified above for the sulfonium cation having formula (cation-1). Examples of the cation in the iodonium salt having formula (3-2) are as exemplified above for the iodonium cation having formula (cation-2).

In formulae (3-1) and (3-2), Xa⁻ is an anion selected from the following formulae (3A) to (3D).

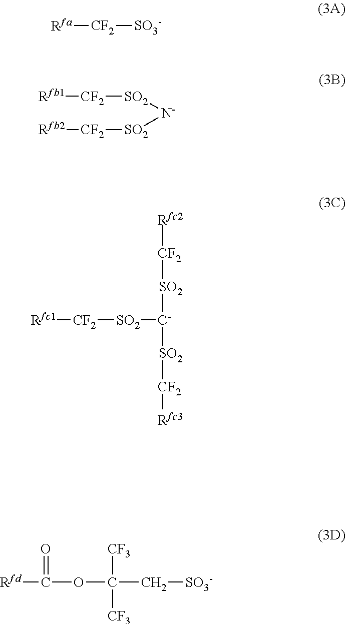

In formula (3A), R^{fa} is fluorine or a C₁-C₄₀ hydrocarbyl group which may contain a heteroatom. The hydrocarbyl group may be saturated or unsaturated and straight, branched or cyclic. Examples thereof are as will be exemplified later for hydrocarbyl group R^{fa1} in formula (3A′).

Of the anions of formula (3A), a structure having formula (3A′) is preferred.

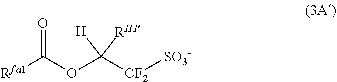

In formula (3A′), R^{HF} is hydrogen or trifluoromethyl, preferably trifluoromethyl.

R^{fa1} is a C₁-C₃₈ hydrocarbyl group which may contain a heteroatom. Suitable heteroatoms include oxygen, nitrogen, sulfur and halogen, with oxygen being preferred. Of the hydrocarbyl groups, those of 6 to 30 carbon atoms are preferred because a high resolution is available in fine pattern formation. The hydrocarbyl group R^{fa1} may be saturated or unsaturated and straight, branched or cyclic. Suitable hydrocarbyl groups include C₁-C₃₈ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, icosyl; C₃-C₃₈ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, dicyclohexylmethyl; C₂-C₃₈ unsaturated aliphatic hydrocarbyl groups such as allyl and 3-cyclohexenyl; C₆-C₃₈ aryl groups such as phenyl, 1-naphthyl, 2-naphthyl; C₇-C₃₈ aralkyl groups such as benzyl and diphenylmethyl; and combinations thereof.

In the hydrocarbyl groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, carbonyl, ether bond, ester bond, sulfonic ester bond, carbonate bond, lactone ring, sultone ring, carboxylic anhydride (—C(═O)—O—C(═O)—) or haloalkyl moiety. Of the heteroatoms, oxygen is preferred. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

With respect to the synthesis of the sulfonium salt having an anion of formula (3A′), reference is made to JP-A 2007-145797, JP-A 2008-106045, JP-A 2009-007327, and JP-A 2009-258695. Also useful are the sulfonium salts described in JP-A 2010-215608, JP-A 2012-041320, JP-A 2012-106986, and JP-A 2012-153644.

Examples of the anion having formula (3A) are shown below, but not limited thereto.

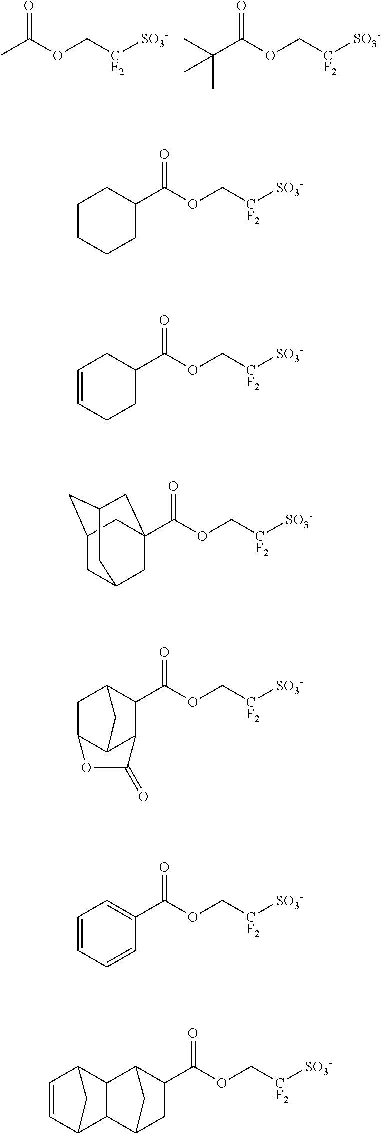

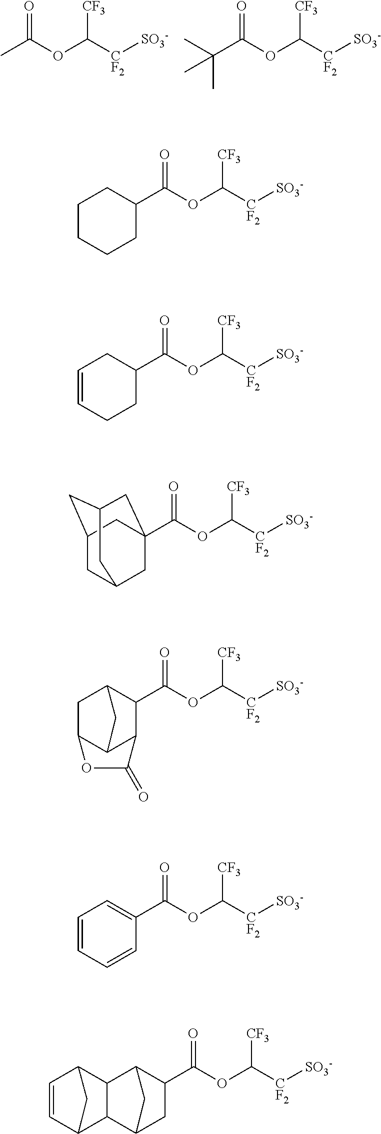

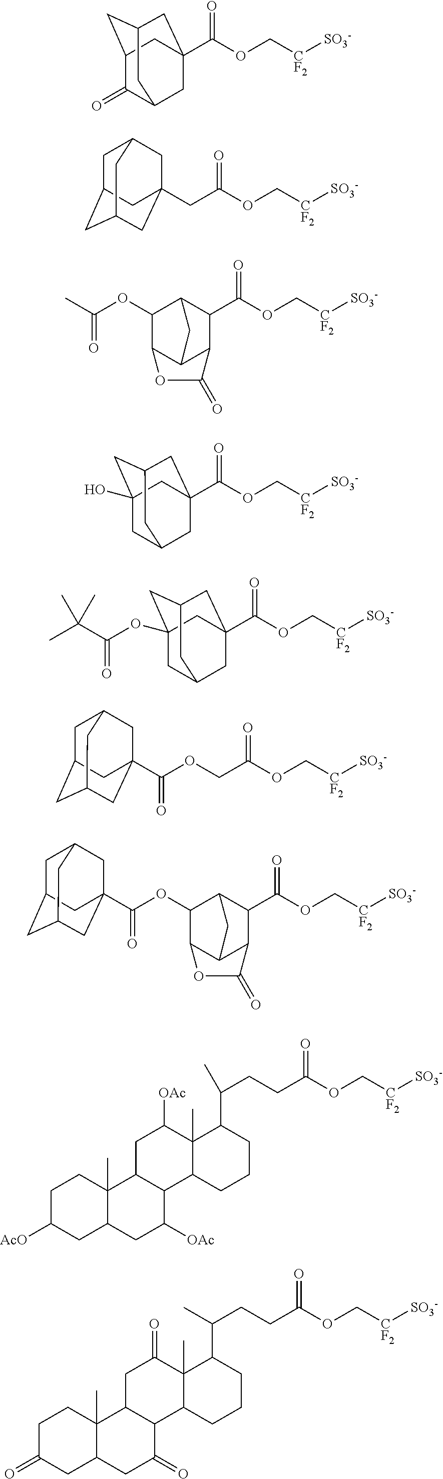

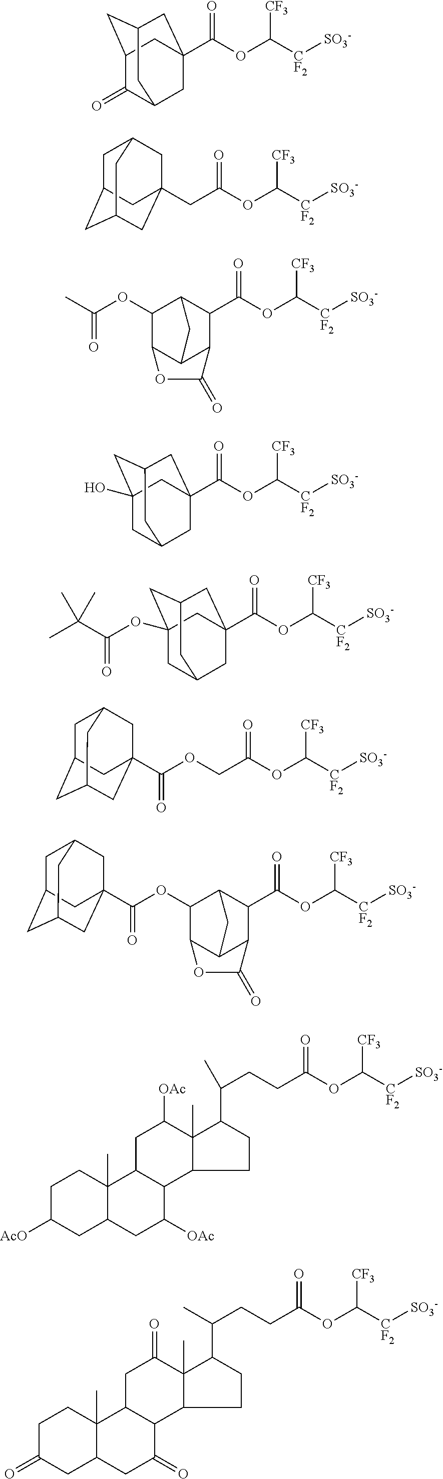

In formula (3B3), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1} in formula (3A′). Preferably R^{fb1} and R^{fb2} each are fluorine or a straight C₁-C₄ fluorinated alkyl group. A pair of R^{fb1} and R^{fb2} may bond together to form a ring with the linkage (—CF₂—SO₂—N—SO₂—CF₂—) to which they are attached, and the R^{fb1}-R^{fb2} group is preferably a fluorinated ethylene or fluorinated propylene group.

In formula (3C), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1} in formula (3A′). Preferably R^{fc1}, R^{fc2} and R^{fc3} each are fluorine or a straight C₁-C₄ fluorinated alkyl group. A pair of R^{fc1} and R^{fc2} may bond together to form a ring with the linkage (—CF₂—SO₂—C⁻—SO₂—CF₂—) to which they are attached, and the R^{fc1}-R^{fc2} group is preferably a fluorinated ethylene or fluorinated propylene group.

In formula (3D), R^{fd} is a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1}.

With respect to the synthesis of the sulfonium salt having an anion of formula (3D), reference is made to JP-A 2010-215608 and JP-A 2014-133723.

Examples of the anion having formula (3D) are shown below, but not limited thereto.

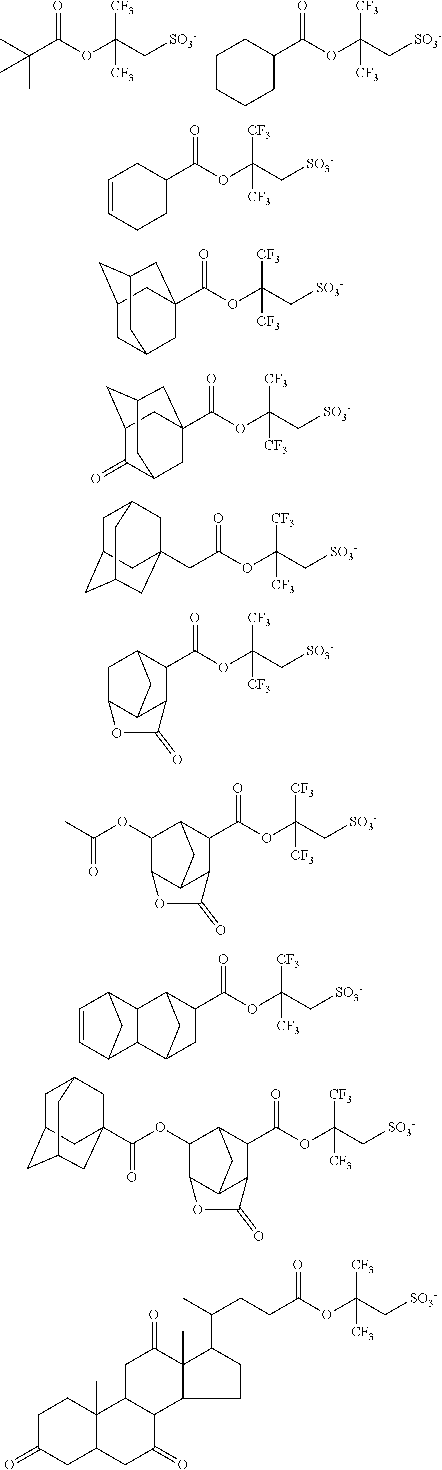

The compound having the anion of formula (3D) has a sufficient acid strength to cleave acid labile groups in the base polymer because it is free of fluorine at α-position of sulfo group, but has two trifluoromethyl groups at β-position. Thus the compound is a useful PAG.

Also compounds having the formula (4) are useful as the PAG.

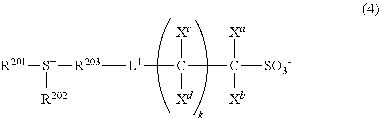

In formula (4), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰², and R²⁰³ may bond together to form a ring with the sulfur atom to which they are attached. Exemplary rings are the same as described above for the ring that R^{ct1} and R^{ct2} in formula (cation-1), taken together, form with the sulfur atom to which they are attached.

The C₁-C₃₀ hydrocarbyl groups R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, isobutyl, sec-butyl, tert-butyl, n-pentyl, tert-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl, and adamantyl; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl; and combinations thereof. In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety.

The C₁-C₃₀ hydrocarbylene group R²⁰³ may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl and adamantanediyl; C₆-C₃₀ arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene and tert-butylnaphthylene; and combinations thereof. In the hydrocarbylene groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonic ester bond, carbonate bond, lactone ring, sultone ring, carboxylic anhydride (—C(═O)—O—C(═O)—) or haloalkyl moiety. Of the heteroatoms, oxygen is preferred.

In formula (4), L¹ is a single bond, ether bond or a C₁-C₂₀ hydrocarbylene group which may contain a heteroatom. The hydrocarbylene group may be saturated or unsaturated and straight, branched or cyclic. Examples thereof are as exemplified above for R²⁰³.

In formula (4), X^{A}, X^{B}, X^{C} and X^{D} are each independently hydrogen, fluorine or trifluoromethyl. At least one of X^{A}, X^{B}, X^{C} and X^{D} is fluorine or trifluoromethyl.

In formula (4), k is an integer of 0 to 3.

Of the PAGs having formula (4), those having formula (4′) are preferred.

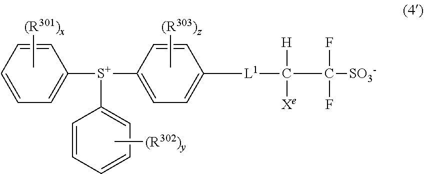

In formula (4′), L¹ is as defined above. X^{e} is hydrogen or trifluoromethyl, preferably trifluoromethyl. R³⁰¹, R³⁰² and R³⁰³ are each independently a C₁-C₂₀ hydrocarbyl group which may contain a heteroatom. The hydrocarbyl group may be saturated or unsaturated and straight, branched or cyclic. Examples thereof are as exemplified above for R^{fa1} in formula (3A′). The subscripts x and y are each independently an integer of 0 to 5, and z is an integer of 0 to 4.

Examples of the PAG having formula (4) are as exemplified for the PAG having formula (2) in JP-A 2017-026980.

Of the foregoing PAGs, those having an anion of formula (3A′) or (3D) are especially preferred because of reduced acid diffusion and high solubility in the solvent. Also those having formula (4′) are especially preferred because of extremely reduced acid diffusion.

Also a sulfonium or iodonium salt having an anion containing an iodized aromatic ring may be used as the PAG. Suitable are sulfonium and iodonium salts having the formulae (5-1) and (5-2).

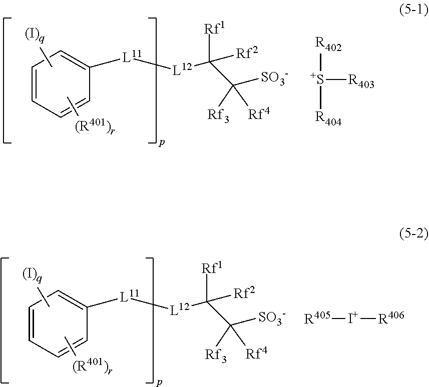

In formulae (5-1) and (5-2), p is 1, 2 or 3, q is an integer of 1 to 5, r is an integer of 0 to 3, and 1≤q+r≤5. Preferably, q is 1, 2 or 3, more preferably 2 or 3, and r is 0, 1 or 2.

L¹¹ is a single bond, ether bond, ester bond, or a C₁-C₆ saturated hydrocarbylene group which may contain an ether bond or ester bond. The saturated hydrocarbylene group may be straight, branched or cyclic.

L¹² is a single bond or a C₁-C₂₀ divalent linking group when p is 1, and a C₁-C₂₀ (p+1)-valent linking group which may contain oxygen, sulfur or nitrogen when p is 2 or 3.

R⁴⁰¹ is a hydroxy group, carboxy group, fluorine, chlorine, bromine, amino group, or a C₁-C₂₀ hydrocarbyl, C₁-C₂₀ hydrocarbyloxy, C₂-C₂₀ hydrocarbylcarbonyl, C₂-C₂₀ hydrocarbyloxycarbonyl, C₂-C₂₀ hydrocarbylcarbonyloxy or C₁-C₂₀ hydrocarbylsulfonyloxy group, which may contain fluorine, chlorine, bromine, hydroxy, amino or ether bond, or —N(R^{401A})(R^{401B})_N(R^{401C})—C(═O)—R^{401D} or —N(R^{401C})—C(═O)—O—R^{401D}. R^{401A} and R^{401B} are each independently hydrogen or a C₁-C₆ saturated hydrocarbyl group. R^{401C} is hydrogen or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{401D} is a C₁-C₁₆ aliphatic hydrocarbyl group, C₆-C₁₄ aryl group or C₇-C₁₅ aralkyl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy, and hydrocarbylsulfonyloxy groups may be straight, branched or cyclic. A plurality of R⁴⁰¹ may be the same or different when p and/or r is 2 or more. Of these, R⁴⁰¹ is preferably hydroxy, —N(R^{401C})—C(═O)—R^{401D}—N(R^{401C})—C(═O)—O—R^{401D}, fluorine, chlorine, bromine, methyl or methoxy.

In formulae (5-1) and (5-2), Rf¹ to Rf⁴ are each independently hydrogen, fluorine or trifluoromethyl, at least one of Rf¹ to Rf⁴ is fluorine or trifluoromethyl. Rf¹ and Rf², taken together, may form a carbonyl group. Preferably, both Rf³ and Rf⁴ are fluorine.

R⁴⁰² to R⁴⁰⁶ are each independently halogen or a C₁-C₂₀ hydrocarbyl group which may contain a heteroatom. The hydrocarbyl group may be saturated or unsaturated and straight, branched or cyclic. Examples thereof include those exemplified above for the hydrocarbyl groups R^{ct1} to R^{ct5} in formulae (cation-1) and (cation-2). In the hydrocarbyl groups, some or all of the hydrogen atoms may be substituted by hydroxy, carboxy, halogen, cyano, nitro, mercapto, sultone ring, sulfo, or sulfonium salt-containing moiety, and some constituent —CH₂— may be replaced by an ether bond, ester bond, carbonyl moiety, amide bond, carbonate bond or sulfonic ester bond. R⁴⁰² and R⁴⁰³ may bond together to form a ring with the sulfur atom to which they are attached. Exemplary rings are the same as described above for the ring that R^{ct1} and R^{ct2} in formula (cation-1), taken together, form with the sulfur atom to which they are attached.

Examples of the cation in the sulfonium salt having formula (5-1) include those exemplified above as the sulfonium cation having formula (cation-1). Examples of the cation in the iodonium salt having formula (5-2) include those exemplified above as the iodonium cation having formula (cation-2).

Examples of the anion in the onium salts having formulae (5-1) and (5-2) are shown below, but not limited thereto.

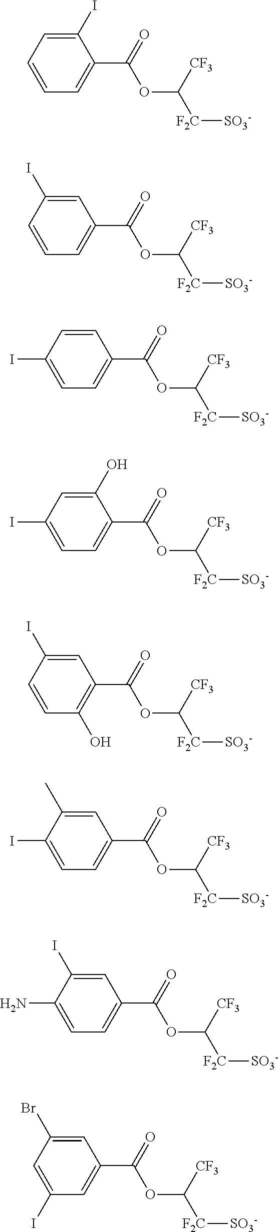

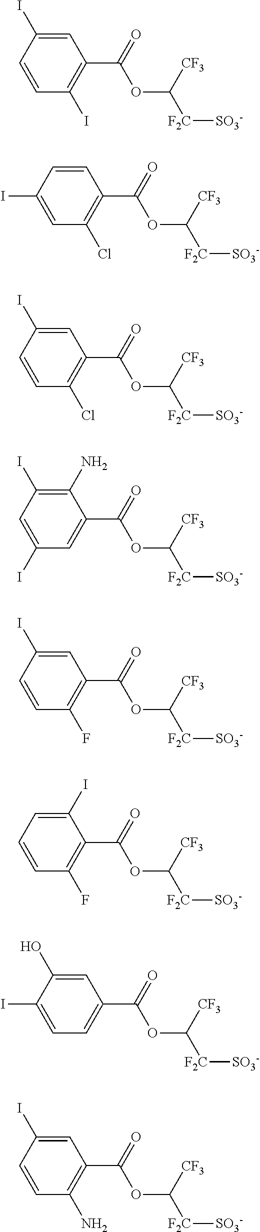

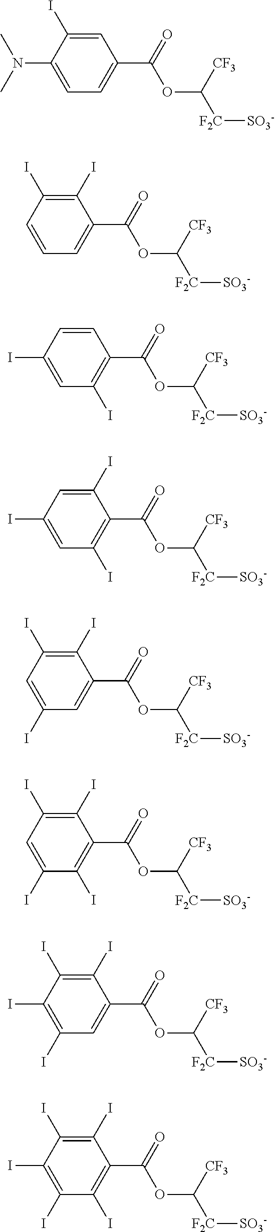

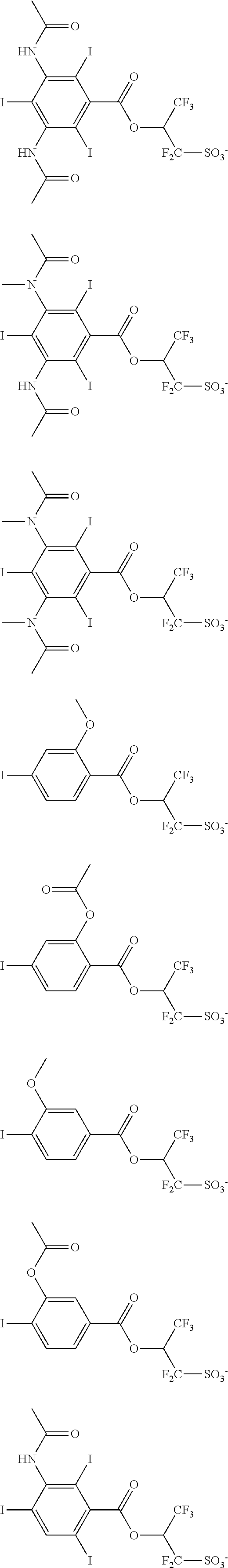

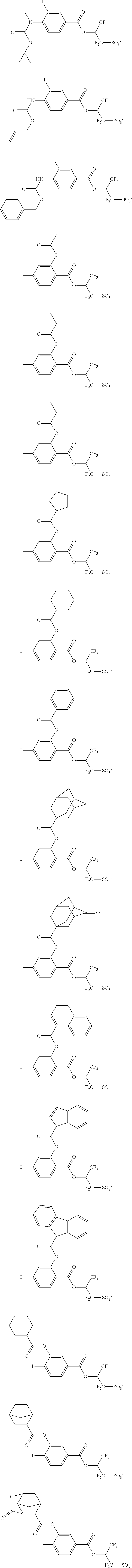

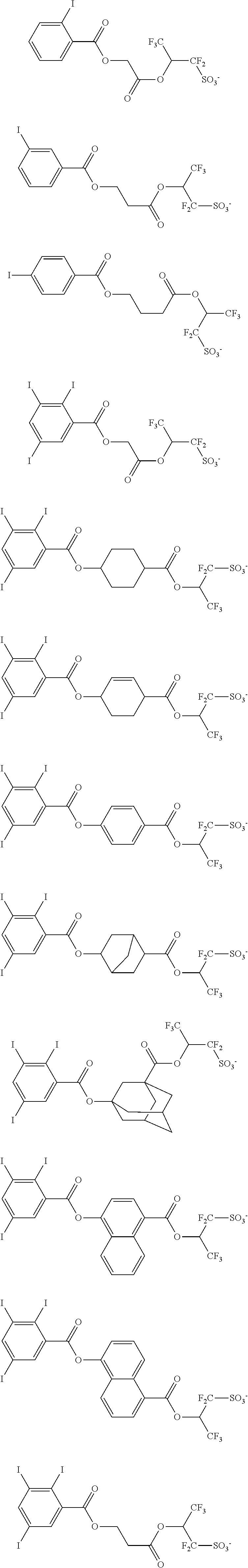

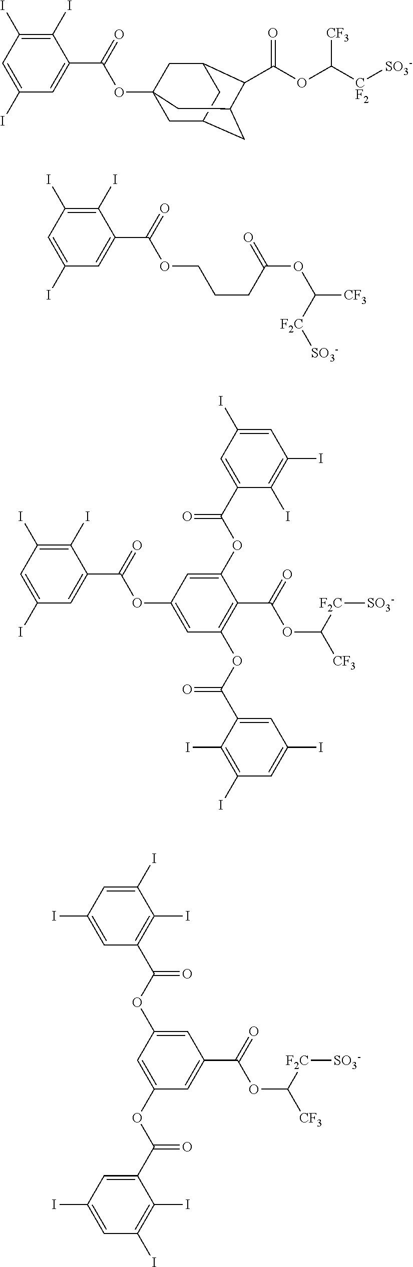

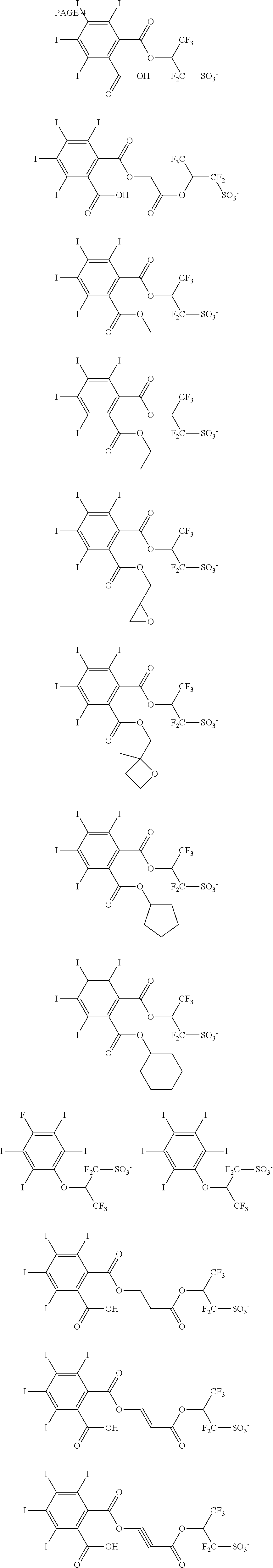

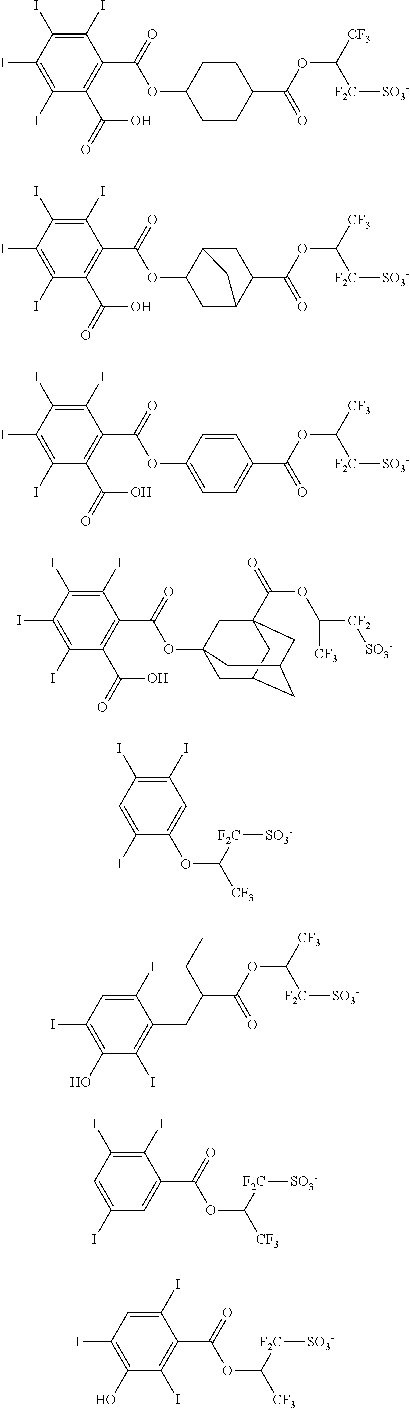

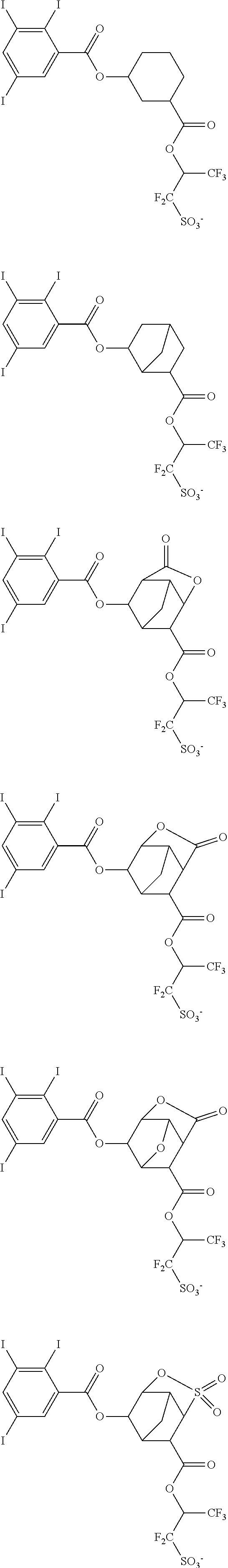

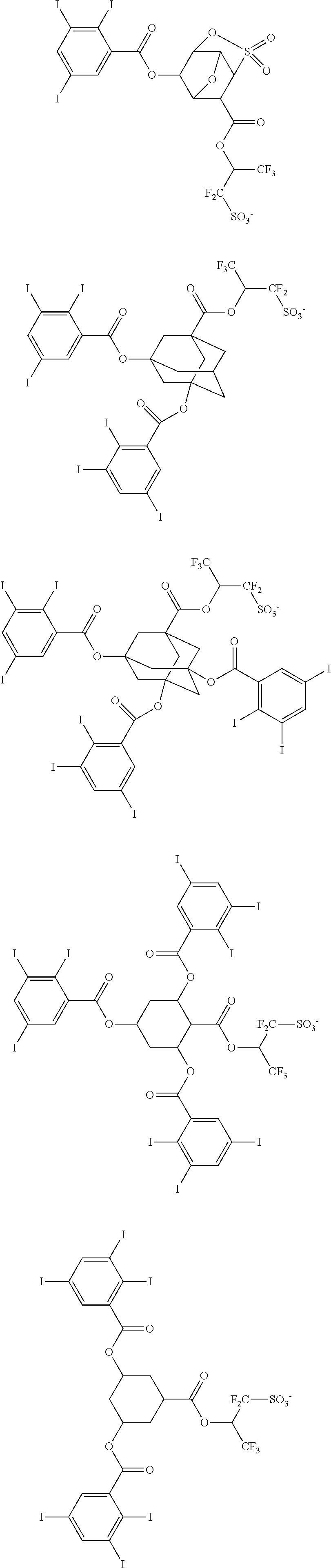

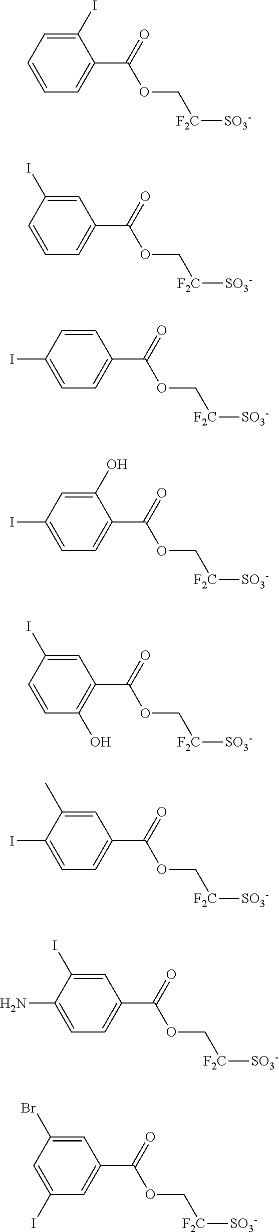

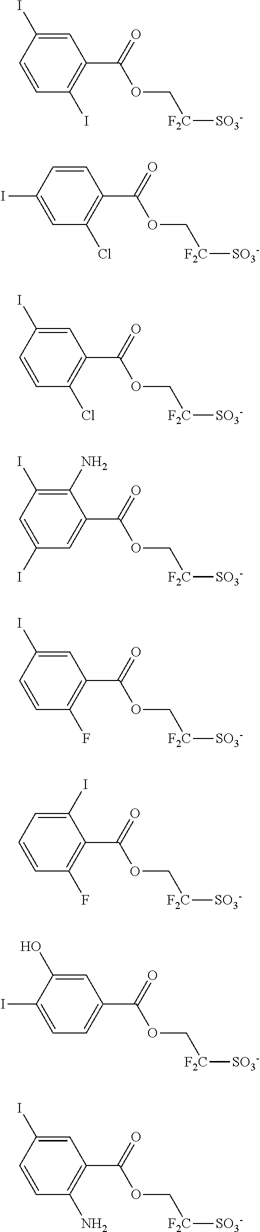

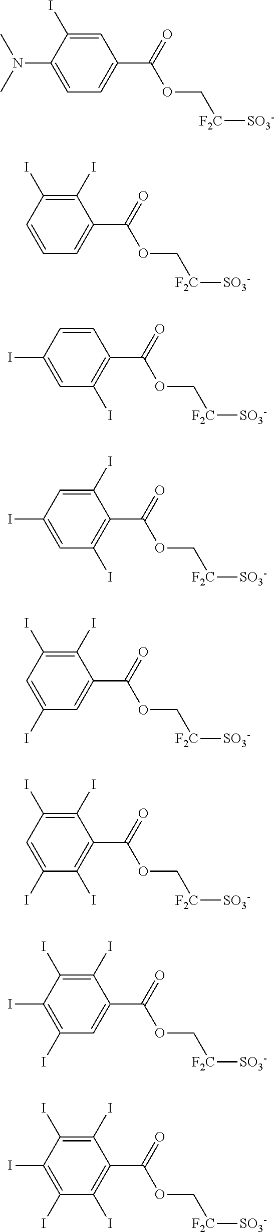

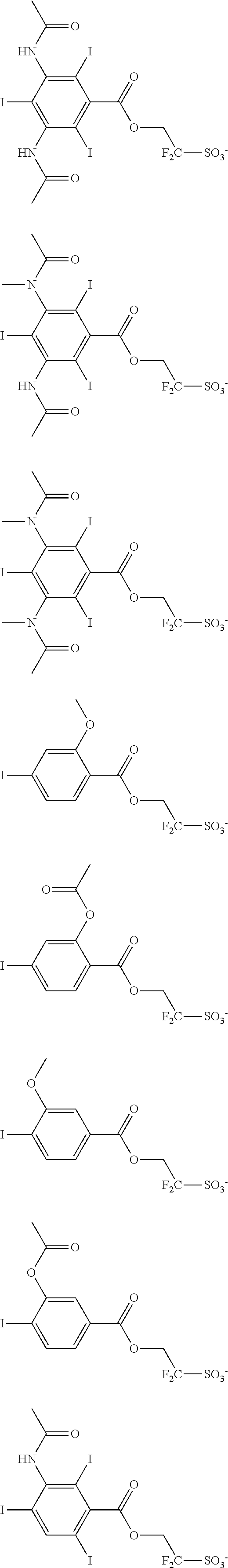

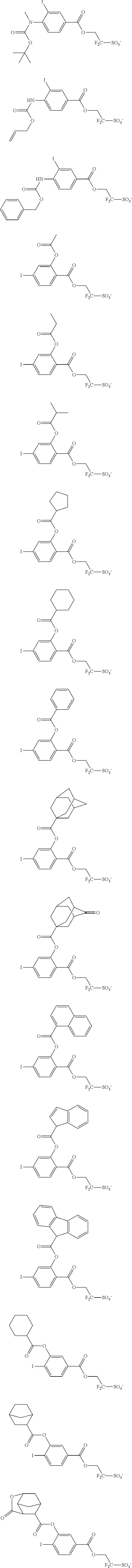

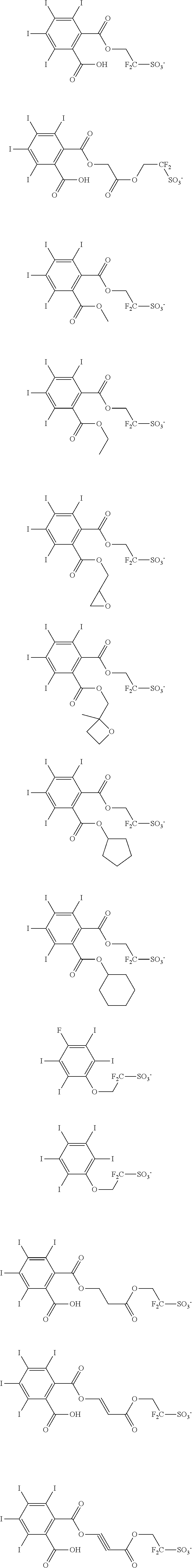

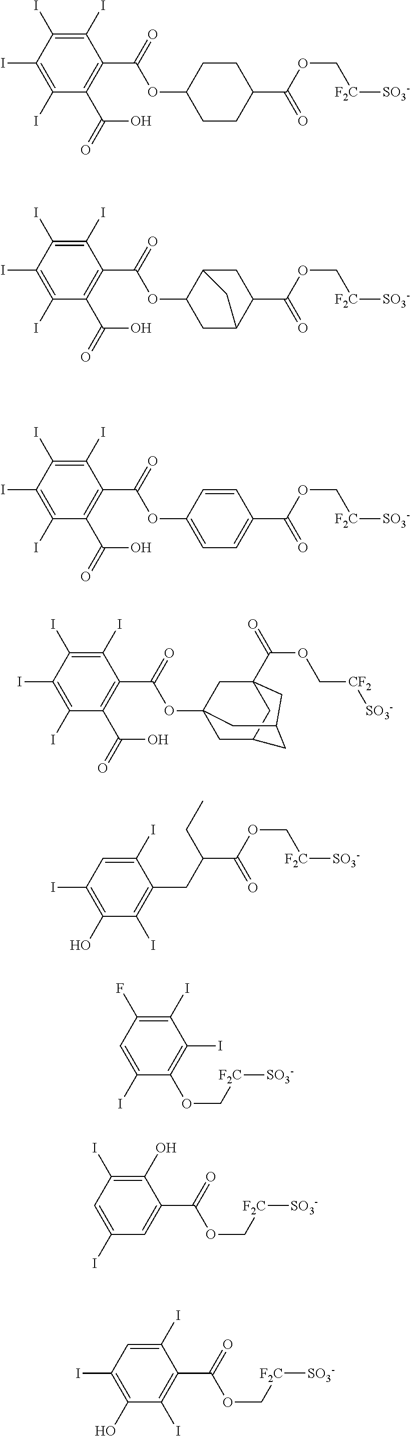

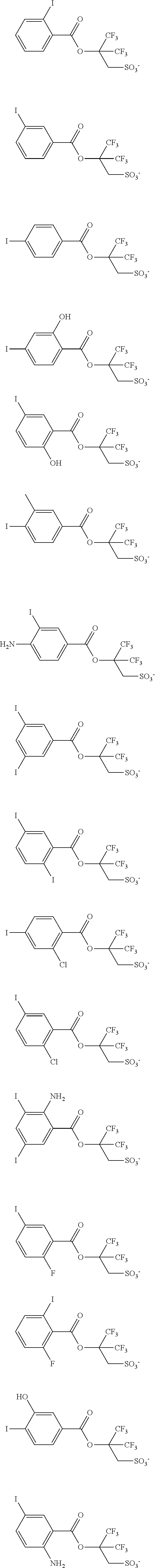

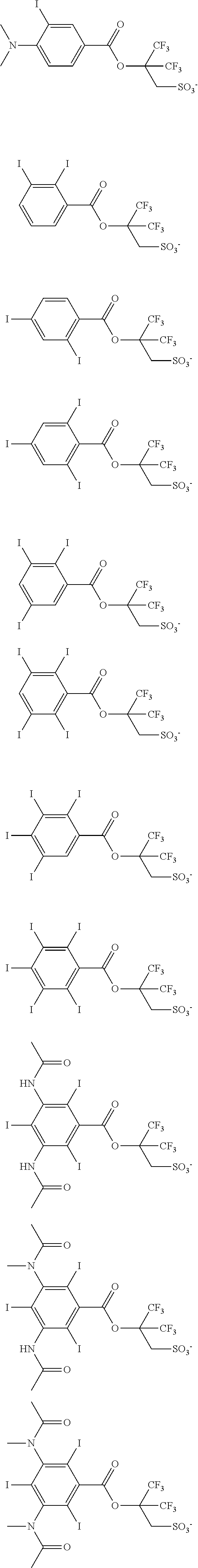

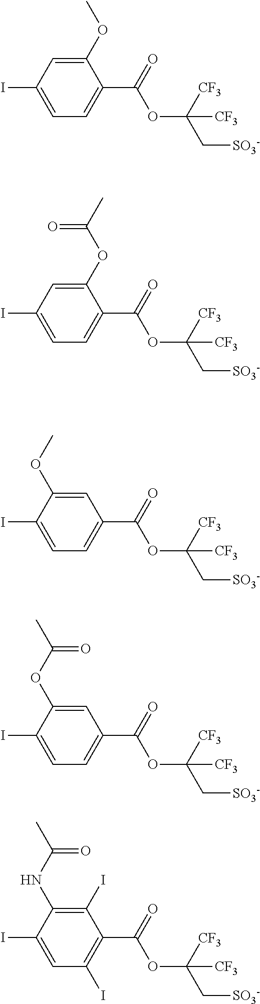

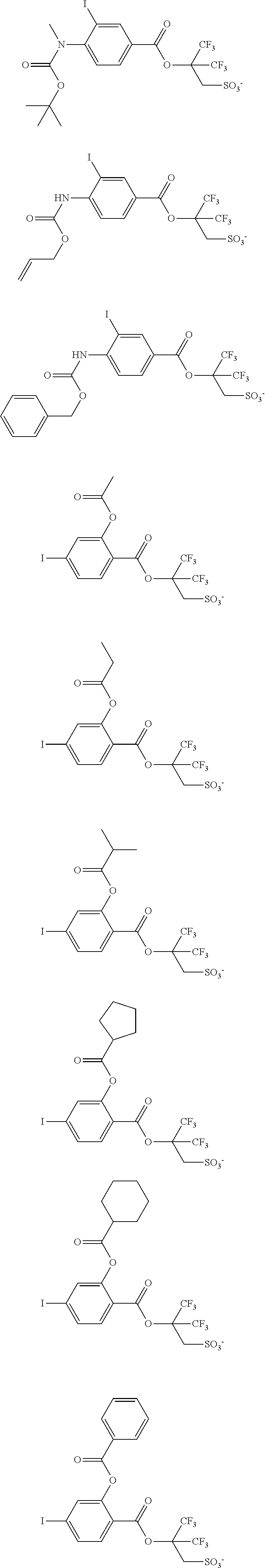

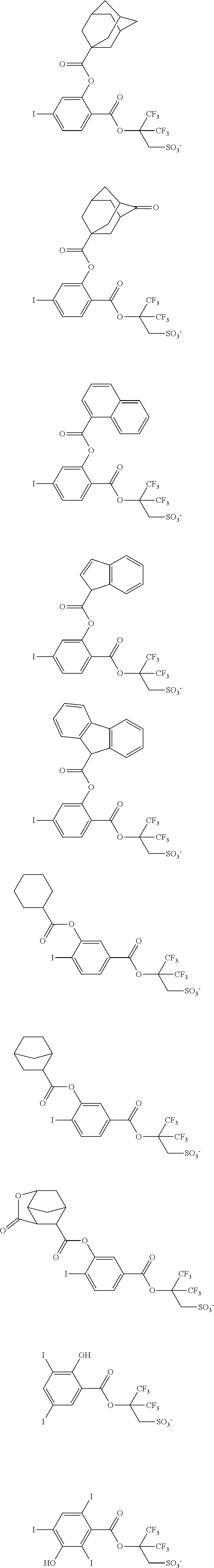

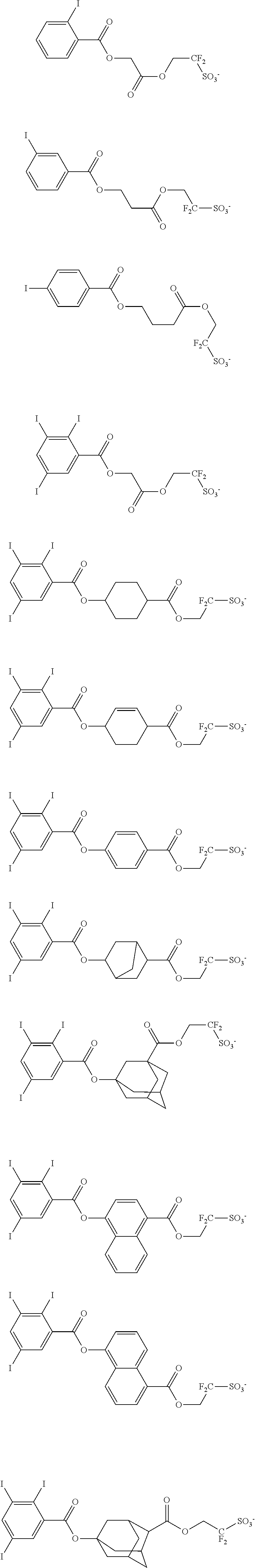

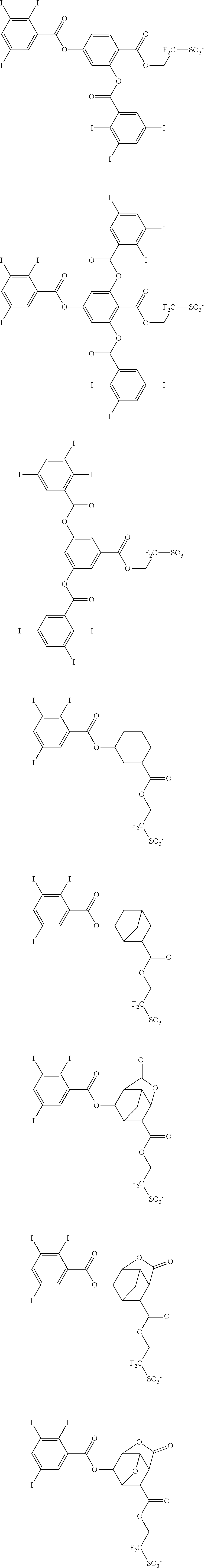

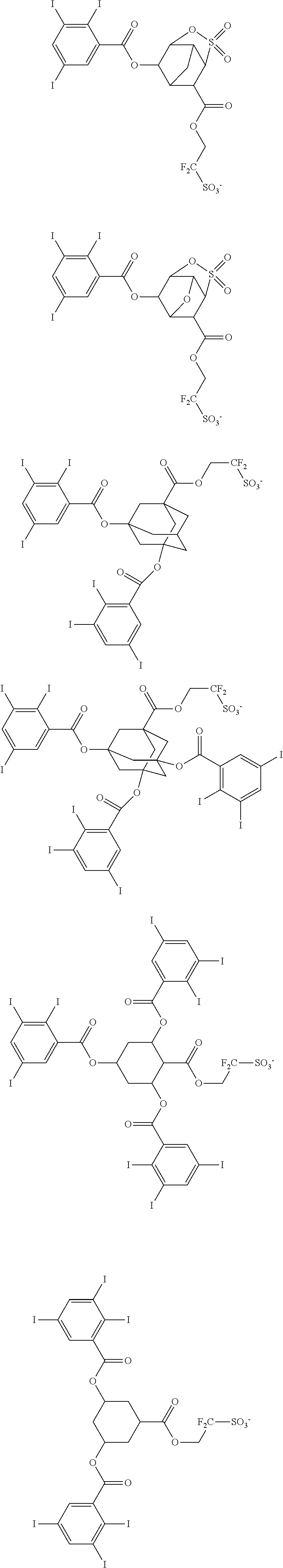

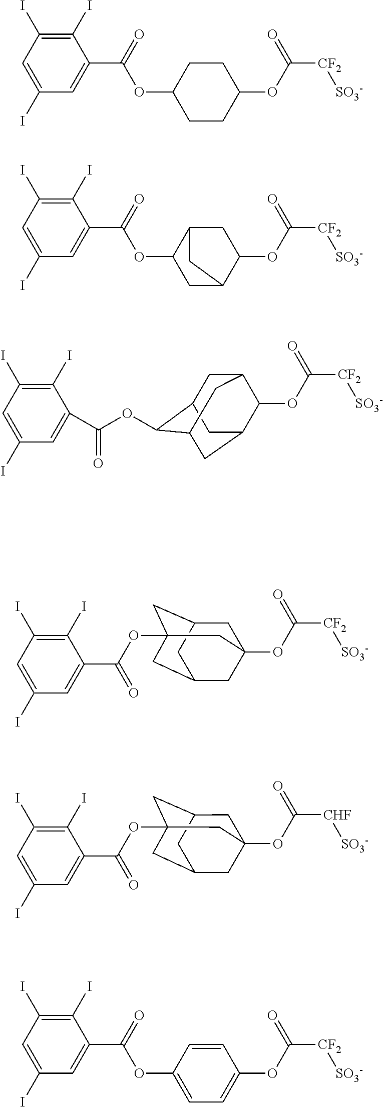

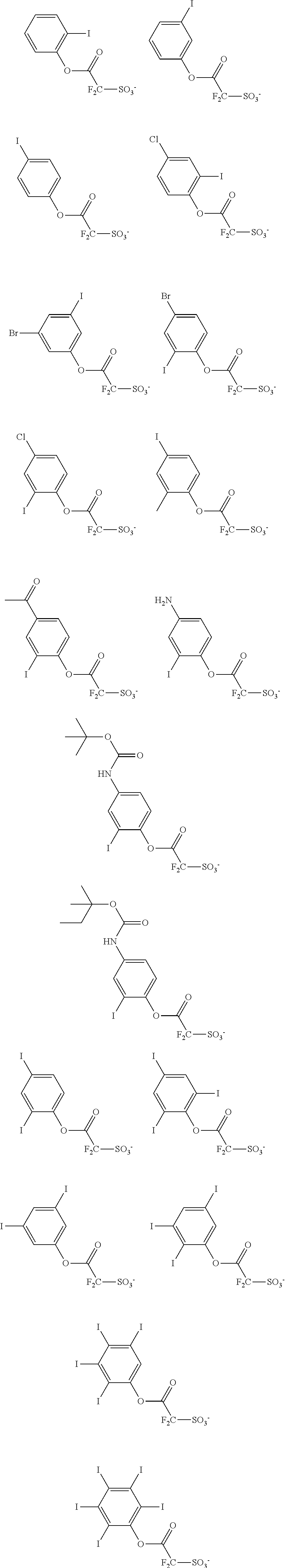

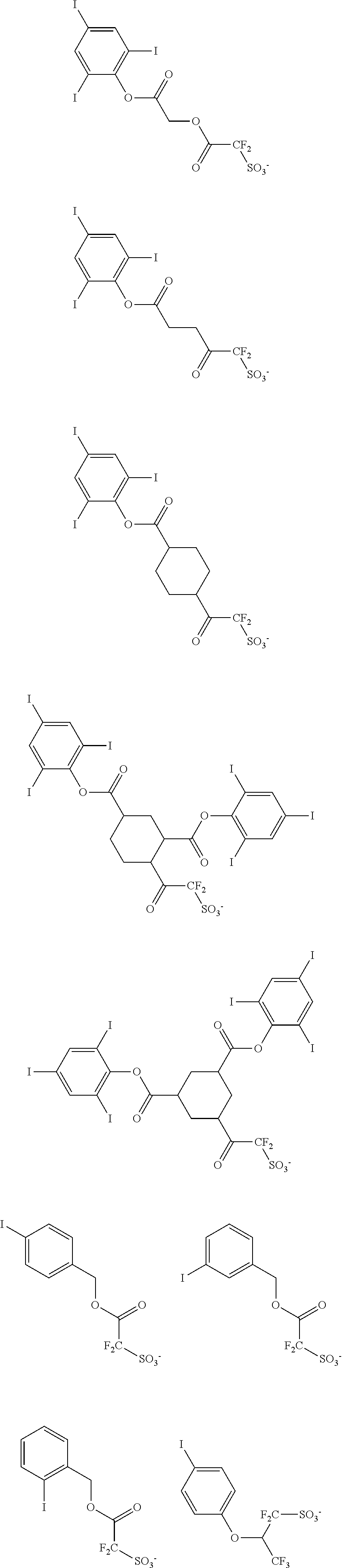

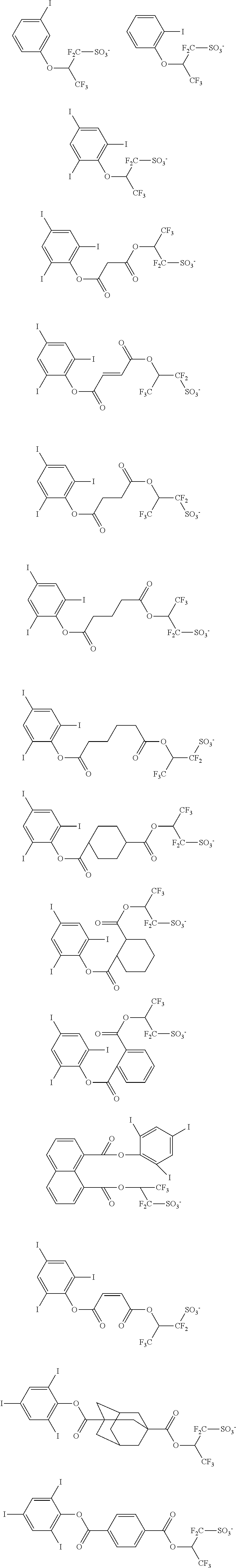

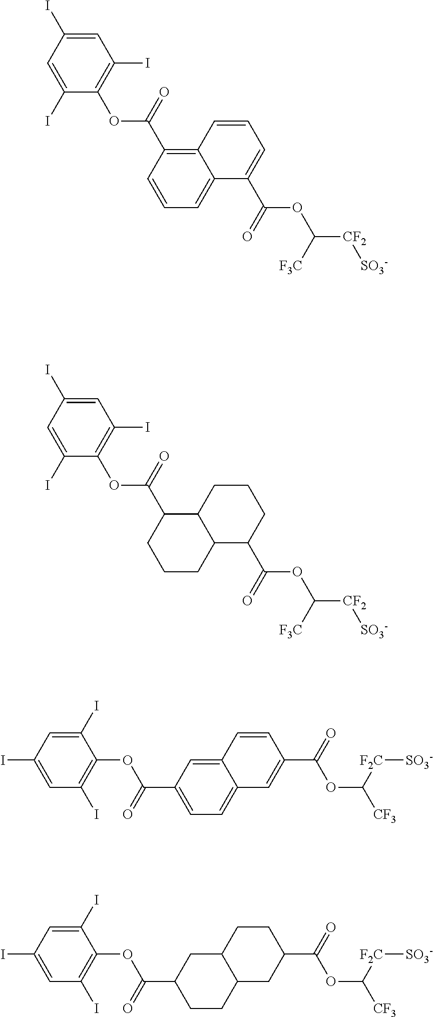

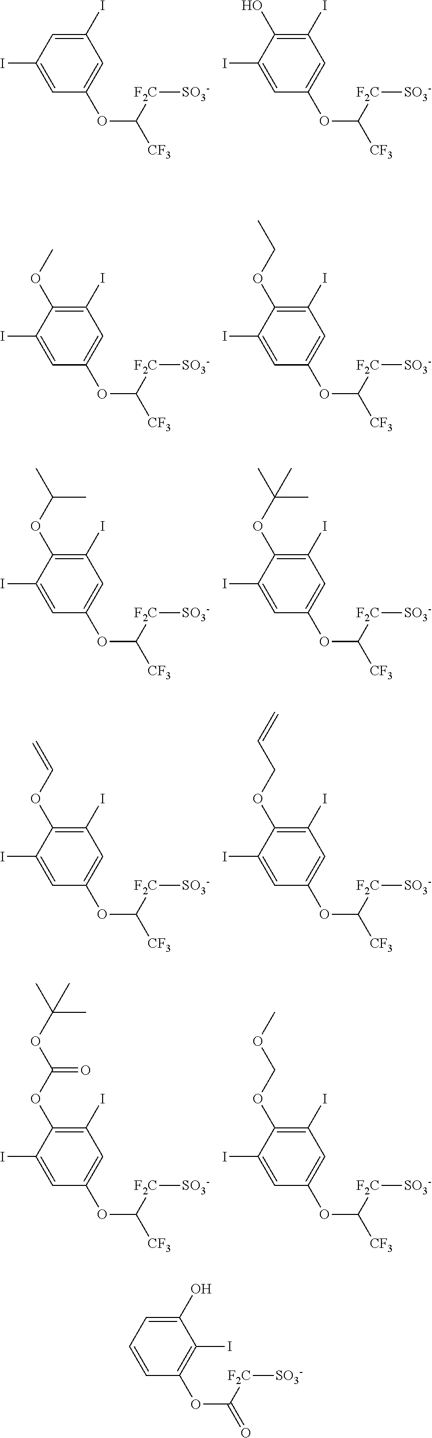

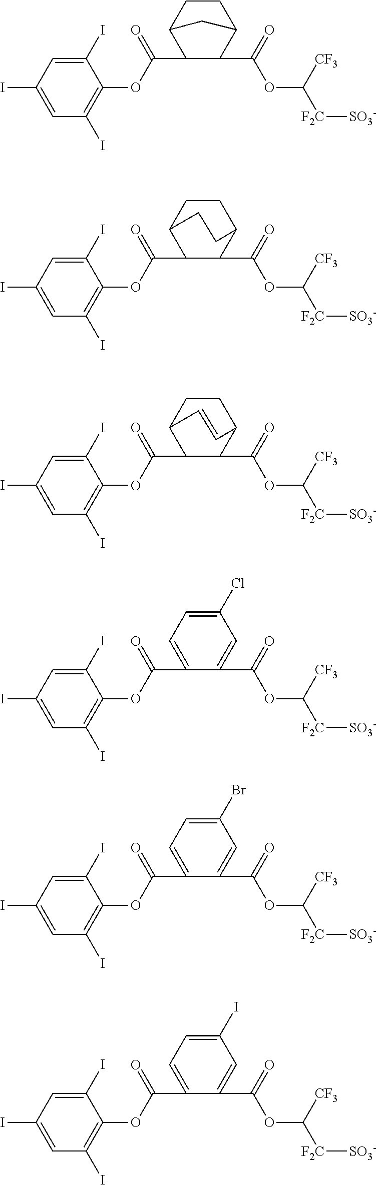

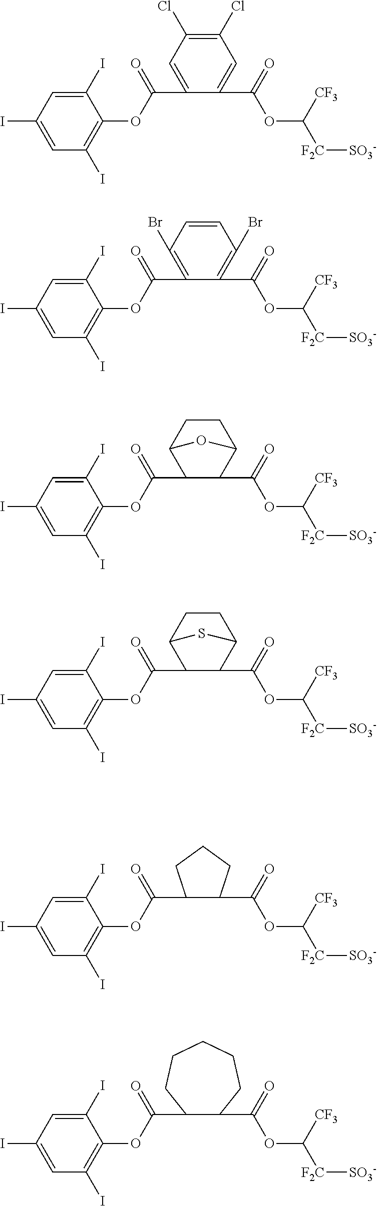

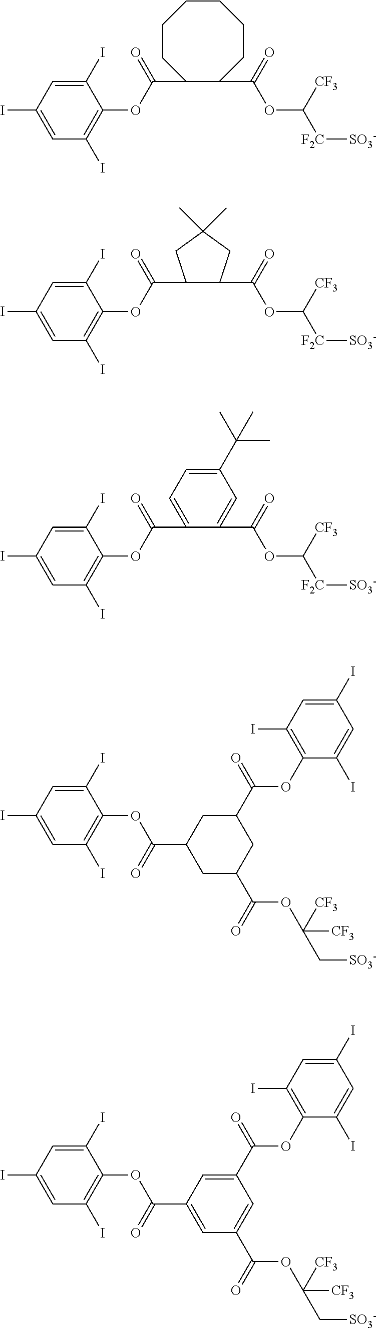

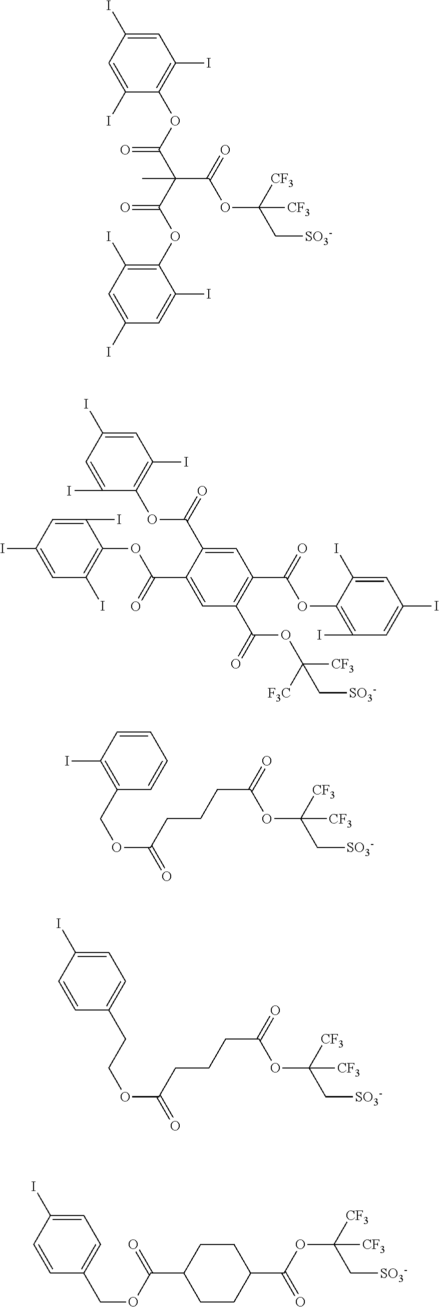

When used, the acid generator (D) is preferably added in an amount of 0.1 to 40 parts, and more preferably 0.5 to 20 parts by weight per 80 parts by weight of the base polymer (A). An amount of the acid generator (D) in the range ensures good resolution and eliminates the risk of leaving foreign particles after development or during separation of resist film. The acid generator may be used alone or in admixture of two or more.

### (E) Surfactant

The resist composition may further comprise (E) a surfactant. It is typically a surfactant which is insoluble or substantially insoluble in water and alkaline developer, or a surfactant which is insoluble or substantially insoluble in water and soluble in alkaline developer. For the surfactant, reference should be made to those compounds described in JP-A 2010-215608 and JP-A 2011-016746.

While many examples of the surfactant which is insoluble or substantially insoluble in water and alkaline developer are described in the patent documents cited herein, preferred examples are fluorochemical surfactants FC-4430 (3M), Olfine® E1004 (Nissin Chemical Co., Ltd.), Surflon® S-381, KH-20 and KH-30 (AGC Seimi Chemical Co., Ltd.). Partially fluorinated oxetane ring-opened polymers having the formula (surf-1) are also useful.

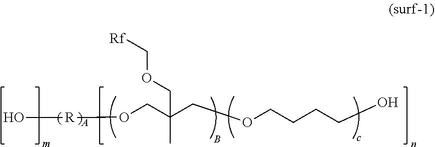

It is provided herein that R, Rf, A, B, C, m, and n are applied to only formula (surf-1), independent of their descriptions other than for the surfactant. R is a di- to tetra-valent C₂-C₅ aliphatic group. Exemplary divalent aliphatic groups include ethylene, 1,4-butylene, 1,2-propylene, 2,2-dimethyl-1,3-propylene and 1,5-pentylene. Exemplary tri- and tetra-valent groups are shown below.

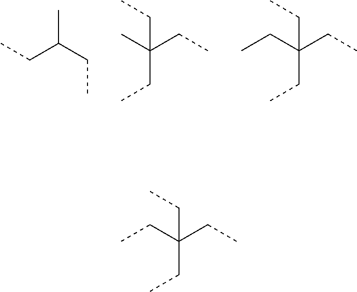

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferred.

Rf is trifluoromethyl or pentafluoroethyl, and preferably trifluoromethyl. The subscript m is an integer of 0 to 3, n is an integer of 1 to 4, and the sum of m and n, which represents the valence of R, is an integer of 2 to 4. “A” is equal to 1, B is an integer of 2 to 25, and C is an integer of 0 to 10. Preferably, B is an integer of 4 to 20, and C is 0 or 1. Note that the formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the surface of a resist film for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Suitable polymeric surfactants include those containing repeat units of at least one type selected from the formulae (6A) to (6E).

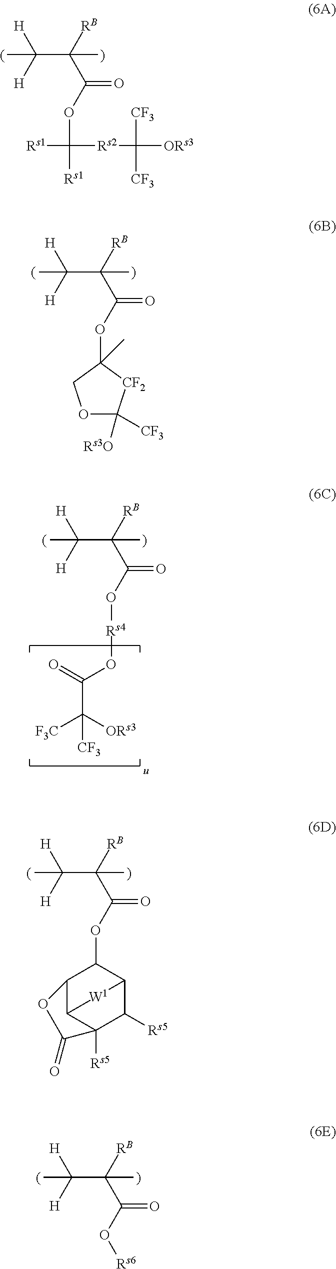

In formulae (6A) to (6E), R^{B} is hydrogen, fluorine, methyl or trifluoromethyl. W is —CH₂—, —CH₂CH₂—, —O—, or two separate —H. R^{s1} is each independently hydrogen or a C₁-C₁₀ hydrocarbyl group. R^{s2} is a single bond or a C₁-C₅ straight or branched hydrocarbylene group. R^{s3} is each independently hydrogen, a C₁-C₁₅ hydrocarbyl or fluorinated hydrocarbyl group, or an acid labile group. When R^{s3} is a hydrocarbyl or fluorinated hydrocarbyl group, an ether bond or carbonyl moiety may intervene in a carbon-carbon bond. R^{s4} is a C₁-C₂₀ (u+1)-valent hydrocarbon or fluorinated hydrocarbon group, and u is an integer of 1 to 3. R^{s5} is each independently hydrogen or a group: —C(═O)—O—R^{sa} wherein R^{sa} is a C₁-C₂₀ fluorinated hydrocarbyl group. R^{s6} is a C₁-C₁₅ hydrocarbyl or fluorinated hydrocarbyl group in which an ether bond or carbonyl moiety may intervene in a carbon-carbon bond.

The hydrocarbyl group R^{s1} is preferably saturated while it may be straight, branched or cyclic. Examples thereof include C₁-C₁₀ alkyl groups such as methyl, ethyl, n-propyl, isopropyl, n-butyl, isobutyl, sec-butyl, tert-butyl, n-pentyl, n-hexyl, n-heptyl, n-octyl, n-nonyl and n-decyl, and C₃-C₁₀ cyclic saturated hydrocarbyl groups such as cyclopropyl, cyclobutyl, cyclopentyl, cyclohexyl, adamantyl and norbornyl. Inter alia, C₁-C₆ groups are preferred.

The hydrocarbylene group R^{s2} is preferably saturated while it may be straight, branched or cyclic. Examples thereof include methylene, ethylene, propylene, butylene, and pentylene.

The hydrocarbyl group R^{s3} or R^{s6} may be saturated or unsaturated and straight, branched or cyclic. Examples thereof include saturated hydrocarbyl groups and aliphatic unsaturated hydrocarbyl groups such as alkenyl and alkynyl groups, with the saturated hydrocarbyl groups being preferred. Suitable saturated hydrocarbyl groups include those exemplified for the hydrocarbyl group represented by R^{s1} as well as undecyl, dodecyl, tridecyl, tetradecyl, and pentadecyl. Examples of the fluorinated hydrocarbyl group represented by R^{s3} or R^{s6} include the foregoing hydrocarbyl groups in which some or all carbon-bonded hydrogen atoms are substituted by fluorine atoms. In these groups, an ether bond or carbonyl moiety may intervene in a carbon-carbon bond as mentioned above.

Examples of the acid labile group represented by R^{s3} include groups of the above formulae (AL-3) to (AL-5), trialkylsilyl groups in which each alkyl moiety has 1 to 6 carbon atoms, and C₄-C₂₀ oxoalkyl groups.

The (u+1)-valent hydrocarbon or fluorinated hydrocarbon group represented by R^{s4} may be straight, branched or cyclic, and examples thereof include the foregoing hydrocarbyl or fluorinated hydrocarbyl groups from which “u” number of hydrogen atoms are eliminated.

The fluorinated hydrocarbyl group represented by R^{sa} is preferably saturated while it may be straight, branched or cyclic. Examples thereof include the foregoing hydrocarbyl groups in which some or all hydrogen atoms are substituted by fluorine atoms. Illustrative examples include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 2-(perfluorodecyl)ethyl.

Examples of the repeat units having formulae (6A) to (6E) are shown below, but not limited thereto. Herein R^{B} is as defined above.

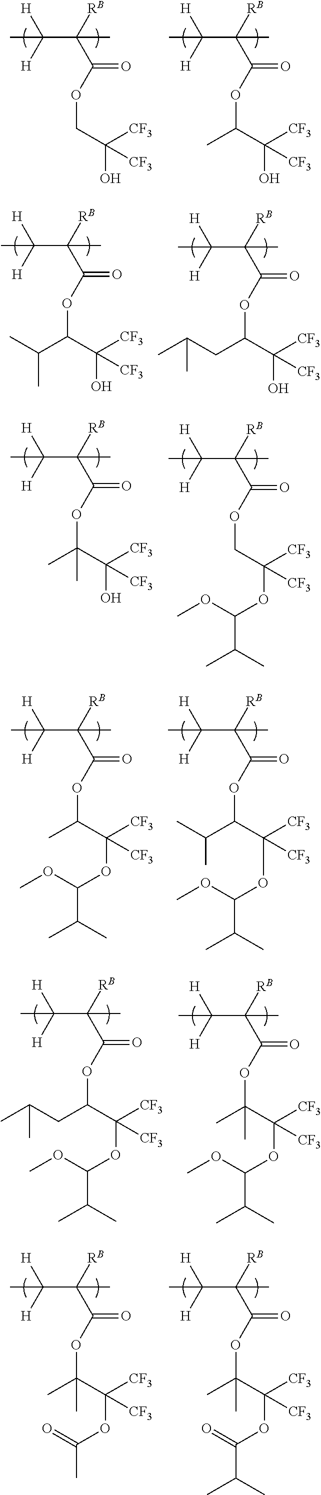

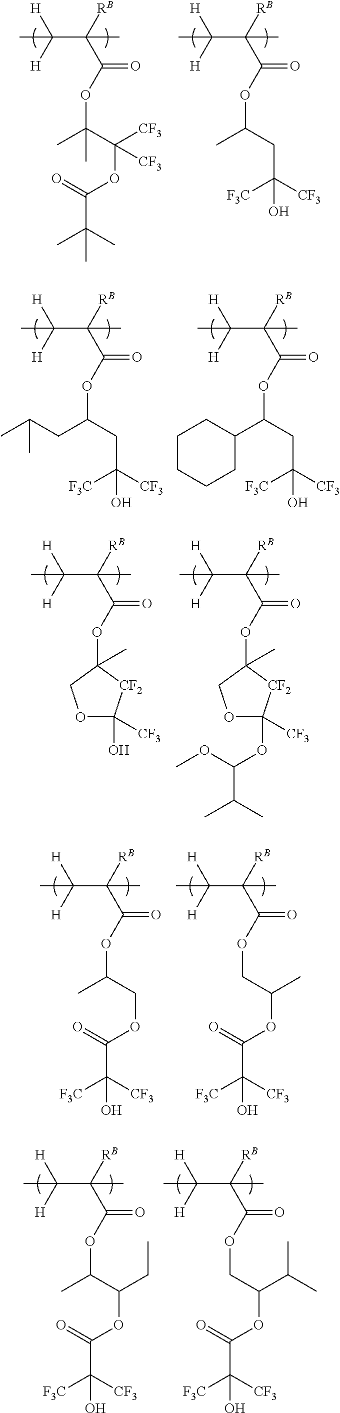

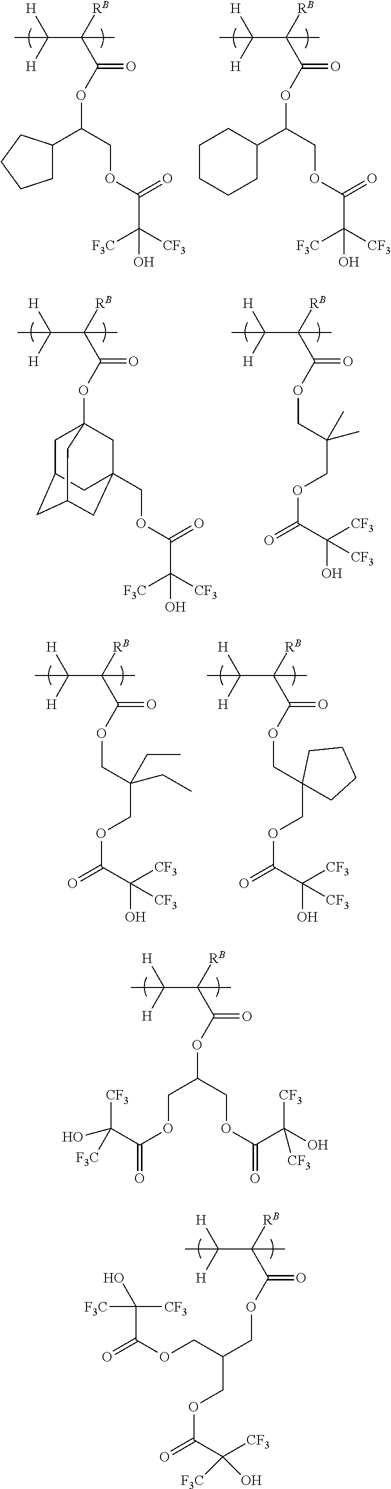

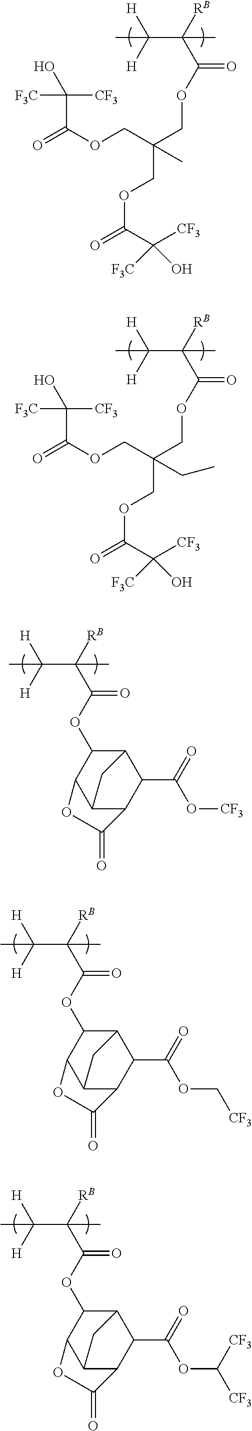

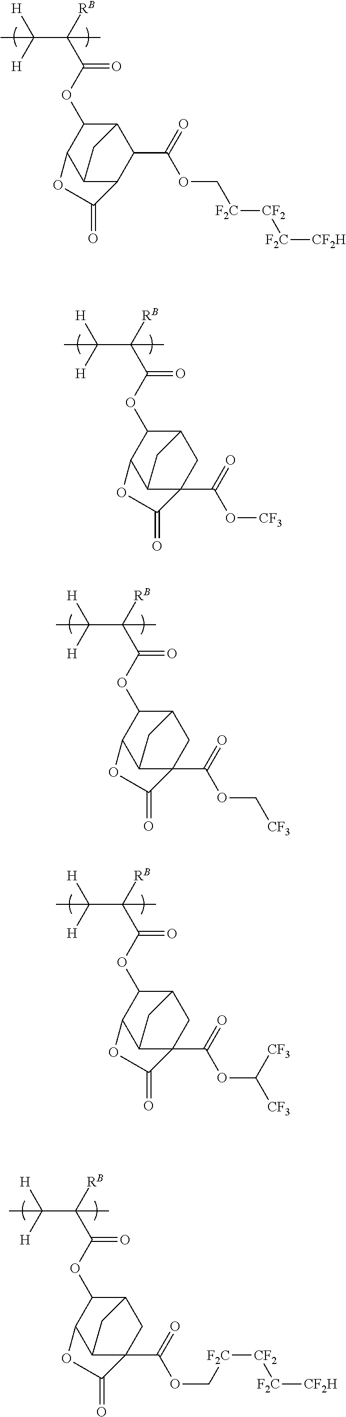

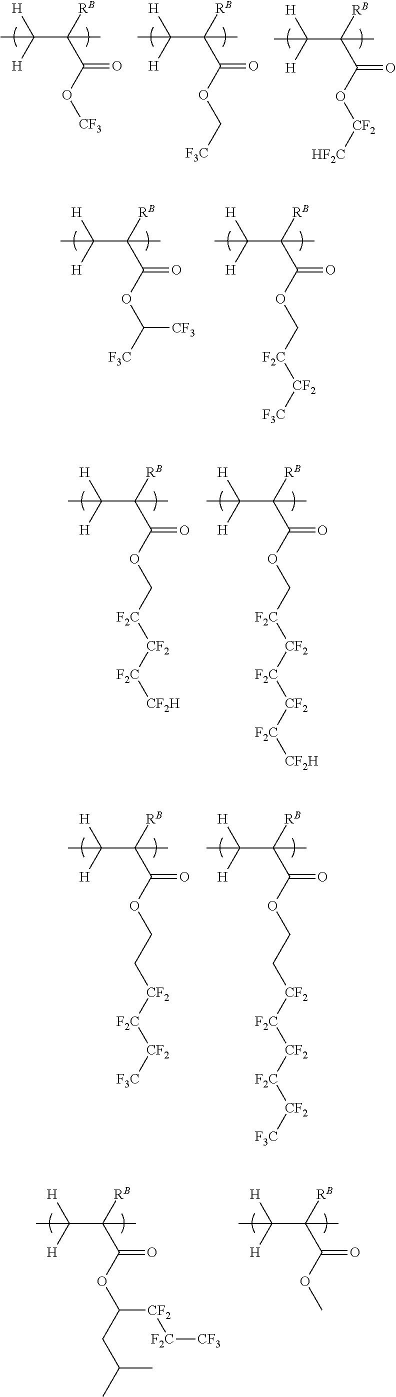

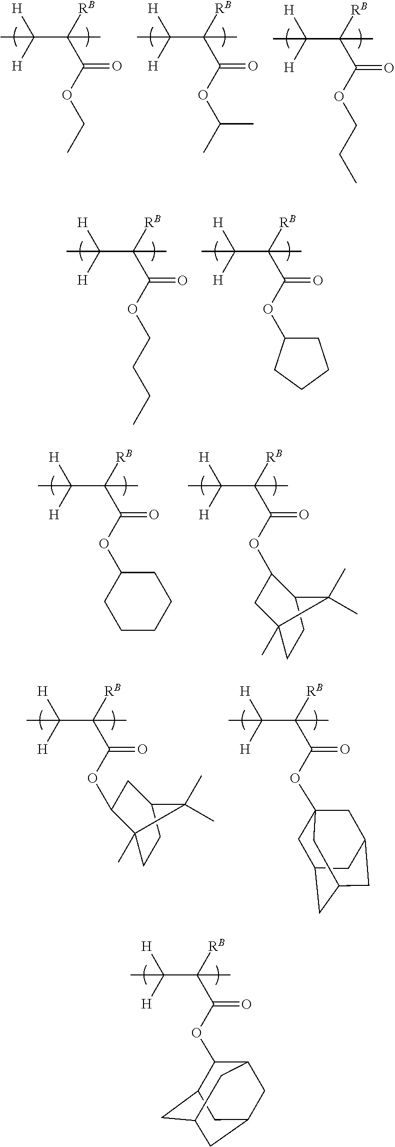

The polymeric surfactant may further contain repeat units other than the repeat units having formulae (6A) to (6E). Typical other repeat units are those derived from methacrylic acid and α-trifluoromethylacrylic acid derivatives. In the polymeric surfactant, the content of the repeat units having formulae (6A) to (6E) is preferably at least 20 mol %, more preferably at least 60 mol %, most preferably 100 mol % of the overall repeat units.

Preferably the polymeric surfactant has a Mw of 1,000 to 500,000, more preferably 3,000 to 100,000 and a Mw/Mn of 1.0 to 2.0, more preferably 1.0 to 1.6.

The polymeric surfactant may be synthesized, for example, by dissolving an unsaturated bond-containing monomer or monomers, from which repeat units having formulae (6A) to (6E) and optional other repeat units are derived, in an organic solvent, adding a radical initiator, and heating for polymerization. Suitable organic solvents used herein include toluene, benzene, THF, diethyl ether, and dioxane. Examples of the polymerization initiator used herein include AIBN, 2,2′-azobis(2,4-dimethylvaleronitrile), dimethyl 2,2-azobis(2-methylpropionate), benzoyl peroxide, and lauroyl peroxide. Preferably the reaction temperature is 50 to 100° C. and the reaction time is 4 to 24 hours. The acid labile group that has been incorporated in the monomer may be kept as such, or the polymer may be protected or partially protected therewith at the end of polymerization.

During the synthesis of the polymeric surfactant, any of well-known chain transfer agents such as dodecylmercaptan and 2-mercaptoethanol may be used for the purpose of adjusting molecular weight. An appropriate amount of the chain transfer agent is 0.01 to 10 mol % based on the total moles of monomers to be polymerized.

When the resist composition contains the surfactant (E), it is preferably used in an amount of 0.1 to 50 parts by weight, more preferably 0.5 to 10 parts by weight per 80 parts by weight of the base polymer (A). As long as the amount of the surfactant is at least 0.1 part by weight, the receding contact angle of resist film surface with water is fully improved. As long as the amount of the surfactant is up to 50 parts by weight, the dissolution rate of resist film surface in developer is so low that the resulting small-size pattern may maintain a sufficient height. The surfactant (E) may be used alone or in admixture.

### (F) Dissolution Inhibitor

The resist composition may further include (F) a dissolution inhibitor. In the case of positive resist compositions, the inclusion of a dissolution inhibitor may lead to an increased difference in dissolution rate between exposed and unexposed areas and a further improvement in resolution.

The dissolution inhibitor which can be used herein is a compound having at least two phenolic hydroxy groups on the molecule, in which an average of from 0 to 100 mol % of all the hydrogen atoms on the phenolic hydroxy groups are replaced by acid labile groups or a compound having at least one carboxy group on the molecule, in which an average of 50 to 100 mol % of all the hydrogen atoms on the carboxy groups are replaced by acid labile groups, both the compounds having a molecular weight of 100 to 1,000, and preferably 150 to 800. Typical are bisphenol A, trisphenol, phenolphthalein, cresol novolac, naphthalenecarboxylic acid, adamantanecarboxylic acid, and cholic acid derivatives in which the hydrogen atom on the hydroxy or carboxy group is replaced by an acid labile group, as described in U.S. Pat. No. 7,771,914 (JP-A 2008-122932, paragraphs [0155]-[0178]).

In the resist composition, the dissolution inhibitor (F) is preferably added in an amount of 0 to 50 parts, more preferably 5 to 40 parts by weight per 80 parts by weight of the base polymer (A).

### (G) Other Components

In addition to the foregoing components, the resist composition may further contain (G) another component, for example, a compound which is decomposed with an acid to generate another acid (i.e., acid amplifier compound), organic acid derivative, fluorinated alcohol, and water repellency improver. Each additional component may be used alone or in admixture of two or more.

The acid amplifier compound is described in JP-A 2009-269953 and JP-A 2010-215608. The acid amplifier compound is preferably used in an amount of 0 to 5 parts, more preferably 0 to 3 parts by weight per 80 parts by weight of the base polymer (A). An extra amount of the acid amplifier compound can make the acid diffusion control difficult and cause degradations to resolution and pattern profile. With respect to the organic acid derivative and fluorinated alcohol, reference should be made to JP-A 2009-269953 and JP-A 2010-215608.

The water repellency improver may be used in the topcoatless immersion lithography. Suitable water repellency improvers include polymers having a fluoroalkyl group and polymers having a specific structure with a 1,1,1,3,3,3-hexafluoro-2-propanol residue and are described in JP-A 2007-297590 and JP-A 2008-111103, for example. The water repellency improver to be added to the resist composition should be soluble in alkaline developers and organic solvent developers. The water repellency improver of specific structure with a 1,1,1,3,3,3-hexafluoro-2-propanol residue is well soluble in the developer. A polymer comprising repeat units having an amino group or amine salt serves as the water repellency improver and is effective for preventing evaporation of acid during PEB, thus preventing any hole pattern opening failure after development. An appropriate amount of the water repellency improver is 0 to 20 parts, preferably 0.5 to 10 parts by weight per 80 parts by weight of the base polymer (A).

### [Process]

The chemically amplified resist composition is used in the fabrication of various integrated circuits. Pattern formation using the resist composition may be performed by well-known lithography processes. The process generally involves the steps of applying the resist composition onto a substrate to form a resist film thereon, exposing the resist film to high-energy radiation, and developing the exposed resist film in a developer. If necessary, any additional steps may be added.

For example, the resist composition is first applied onto a substrate on which an integrated circuit is to be formed (e.g., Si, SiO₂, SiN, SiON, TiN, WSi, BPSG, SOG, or organic antireflective coating) or a substrate on which a mask circuit is to be formed (e.g., Cr, CrO, CrON, MoSi₂, or SiO₂) by a suitable coating technique such as spin coating, roll coating, flow coating, dipping, spraying or doctor coating. The coating is prebaked on a hotplate at a temperature of preferably 60 to 150° C. for 10 seconds to 30 minutes, more preferably at 80 to 120° C. for 30 seconds to 20 minutes. The resulting resist film is generally 0.01 to 2.0 μm thick.

Then the resist film is exposed to high-energy radiation. Examples of the high-energy radiation include UV, deep-UV, EB, EUV of wavelength 3 to 15 nm, x-ray, soft x-ray, excimer laser light, γ-ray or synchrotron radiation. On use of UV, deep UV, EUV, x-ray, soft x-ray, excimer laser, γ-ray or synchrotron radiation, the resist film is exposed directly or through a mask having a desired pattern, preferably in a dose of about 1 to 200 mJ/cm², more preferably about 10 to 100 mJ/cm². On use of EB, a pattern may be written directly or through a mask having a desired pattern, preferably in a dose of about 0.1 to 100 μC/cm², more preferably about 0.5 to 50 μC/cm². The resist composition is suited for micropatterning using high-energy radiation such as KrF excimer laser of wavelength 248 nm, ArF excimer laser of wavelength 193 nm, EB, EUV of wavelength 3 to 15 nm, x-ray, soft x-ray, γ-ray or synchrotron radiation.

After the exposure, the resist film may be baked (PEB) on a hotplate at 60 to 150° C. for 10 seconds to 30 minutes, preferably at 80 to 120° C. for 30 seconds to 20 minutes.

After the exposure or PEB, the resist film is developed with a developer in the form of an aqueous base solution for 3 seconds to 3 minutes, preferably 5 seconds to 2 minutes by conventional techniques such as dip, puddle and spray techniques. A typical developer is a 0.1 to 10 wt %, preferably 2 to 5 wt % aqueous solution of tetramethylammonium hydroxide (TMAH), tetraethylammonium hydroxide (TEAH), tetrapropylammonium hydroxide (TPAH), or tetrabutylammonium hydroxide (TBAH). The resist film in the exposed area is dissolved in the developer whereas the resist film in the unexposed area is not dissolved. In this way, the desired positive pattern is formed on the substrate.

In an alternative embodiment, a negative pattern may be formed via organic solvent development. The developer used herein is preferably selected from among 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate, and mixtures thereof.

At the end of development, the resist film is rinsed. As the rinsing liquid, a solvent which is miscible with the developer and does not dissolve the resist film is preferred. Suitable solvents include alcohols of 3 to 10 carbon atoms, ether compounds of 8 to 12 carbon atoms, alkanes, alkenes, and alkynes of 6 to 12 carbon atoms, and aromatic solvents. Specifically, suitable alcohols of 3 to 10 carbon atoms include n-propyl alcohol, isopropyl alcohol, 1-butyl alcohol, 2-butyl alcohol, isobutyl alcohol, t-butyl alcohol, 1-pentanol, 2-pentanol, 3-pentanol, t-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol. Suitable ether compounds of 8 to 12 carbon atoms include di-n-butyl ether, diisobutyl ether, di-s-butyl ether, di-n-pentyl ether, diisopentyl ether, di-s-pentyl ether, di-t-pentyl ether, and di-n-hexyl ether. Suitable alkanes of 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Suitable alkenes of 6 to 12 carbon atoms include hexene, heptene, octene, cyclohexene, methylcyclohexene, dimethylcyclohexene, cycloheptene, and cyclooctene. Suitable alkynes of 6 to 12 carbon atoms include hexyne, heptyne, and octyne. Suitable aromatic solvents include toluene, xylene, ethylbenzene, isopropylbenzene, t-butylbenzene and mesitylene. The solvents may be used alone or in admixture.

Rinsing is effective for minimizing the risks of resist pattern collapse and defect formation. However, rinsing is not essential. If rinsing is omitted, the amount of solvent used may be reduced.

A hole or trench pattern after development may be shrunk by the thermal flow, RELACS® or DSA process. A hole pattern is shrunk by coating a shrink agent thereto, and baking such that the shrink agent may undergo crosslinking at the resist surface as a result of the acid catalyst diffusing from the resist layer during bake, and the shrink agent may attach to the sidewall of the hole pattern. The bake is preferably at a temperature of 70 to 180° C., more preferably 80 to 170° C., for a time of 10 to 300 seconds. The extra shrink agent is stripped and the hole pattern is shrunk.

### EXAMPLES

Examples of the invention are given below by way of illustration and not by way of limitation. The abbreviation “pbw” is parts by weight. Analysis is made by time-of-flight mass spectrometry (TOF-MS) using the instrument: MALDI TOF-MS S3000 by JEOL Ltd.

### [1] Synthesis of Onium Salt Monomers

### [Example 1-1] Synthesis of Onium Salt Monomer γ-1

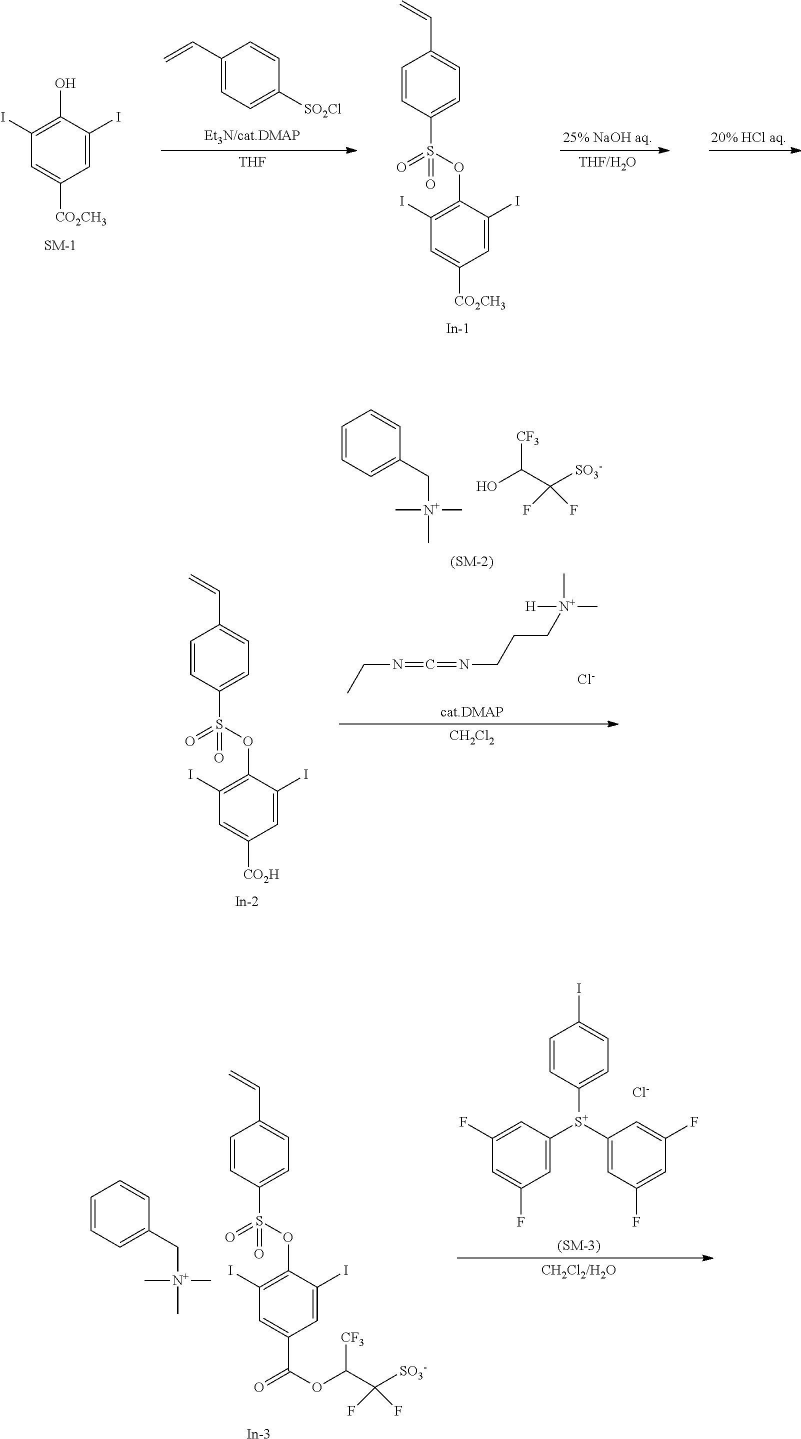

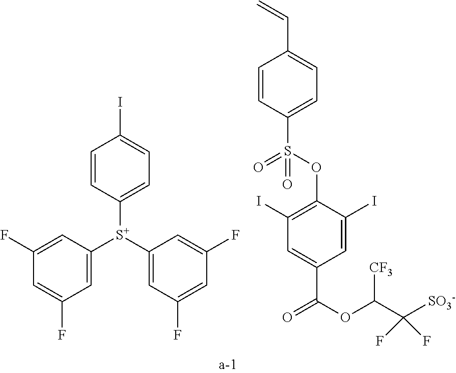

### (1) Synthesis of Intermediate In-1

In a reactor under nitrogen atmosphere, 37.2 g of reactant SM-1, 18.0 g of triethylamine, and 1.50 g of 4-dimethylaminopyridine (DMAP) were dissolved or suspended in 200 g of THF and cooled in an ice bath. A solution of 16.4 g of p-styrenesulfonyl chloride in 50 g of THF was added dropwise thereto. At the end of addition, the reaction mixture was heated at 50° C. and aged for 6 hours. The reaction solution was cooled, after which 100 g of saturated sodium bicarbonate water was added to quench the reaction. The end product was extracted twice with 200 g of ethyl acetate. This was followed by ordinary aqueous work-up, solvent distillation, and purification by silica gel chromatography. Intermediate In-1 was obtained as oily matter (amount 45.8 g, yield 97%).

### (2) Synthesis of Intermediate In-2

In nitrogen atmosphere, 45.8 g of Intermediate In-1 was dissolved in 200 g of THF. While the solution was cooled in an ice bath, 15.5 g of 25 wt % sodium hydroxide aqueous solution was added dropwise. At the end of addition, the solution in the reactor was heated at 30° C. and aged for 12 hours. At the end of aging, the reaction solution was cooled and 18.2 g of 20 wt % hydrochloric acid was added dropwise to quench the reaction. The end product was extracted twice with 200 g of ethyl acetate. This was followed by ordinary aqueous work-up, solvent distillation, and recrystallization from hexane. Intermediate In-2 was obtained as white crystals (amount 37.5 g, yield 90%).

### (3) Synthesis of Intermediate In-3

In nitrogen atmosphere, a reactor was charged with 37.5 g of Intermediate In-2, 34.8 g of reactant SM-2, 1.1 g of 4-dimethylaminopyridine (DMAP), and 150 g of methylene chloride and cooled in an ice bath. While the reactor temperature was kept below 20° C., 20.1 g of 1-ethyl-3-(3-dimethylaminopropyl)carbodiimide hydrochloride was added in powder form. At the end of addition, the reaction mixture was warmed up to room temperature and aged for 12 hours. At the end of aging, 100 g of water was added to quench the reaction. This was followed by ordinary aqueous work-up, solvent distillation, and recrystallization from diisopropyl ether. Intermediate In-3 was obtained as oily matter (amount 61.5 g, yield 89%).

### (4) Synthesis of Onium Salt Monomer a-1

In nitrogen atmosphere, a reactor was charged with 61.5 g of Intermediate In-3, 36.6 g of reactant SM-3, 200 g of methylene chloride, and 150 g of water. The mixture was stirred for 30 minutes. The organic layer was taken out, washed with water, and concentrated under reduced pressure. The concentrate was purified by silica gel chromatography. The target compound, Onium Salt Monomer a-1 was obtained as oily matter (amount 71.6 g, yield 87%).

### MALDI TOF-MS:

positive M⁺ 461 (corresponding to C₁₈H₁₀F₄IS⁺)
negative M⁻ 766 (corresponding to C₁₈H₁₀F₅I₂O₈S₂⁻)

### [Examples 1-2 to 1-7] Synthesis of Onium Salt Monomers a-2 to a-7

Onium Salt Monomers a-2 to a-7, shown below, were synthesized using the corresponding reactants and well-known organic synthesis reaction.

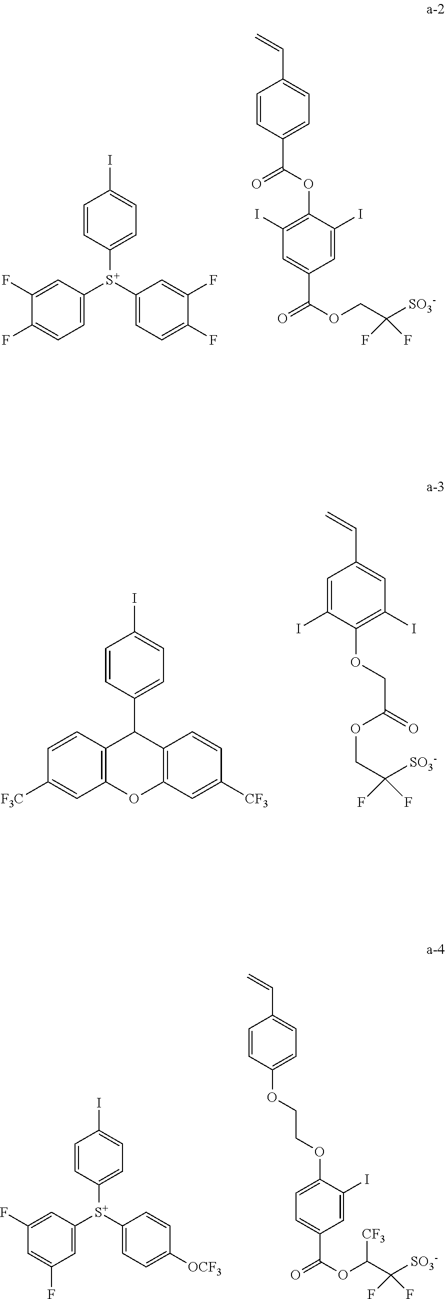

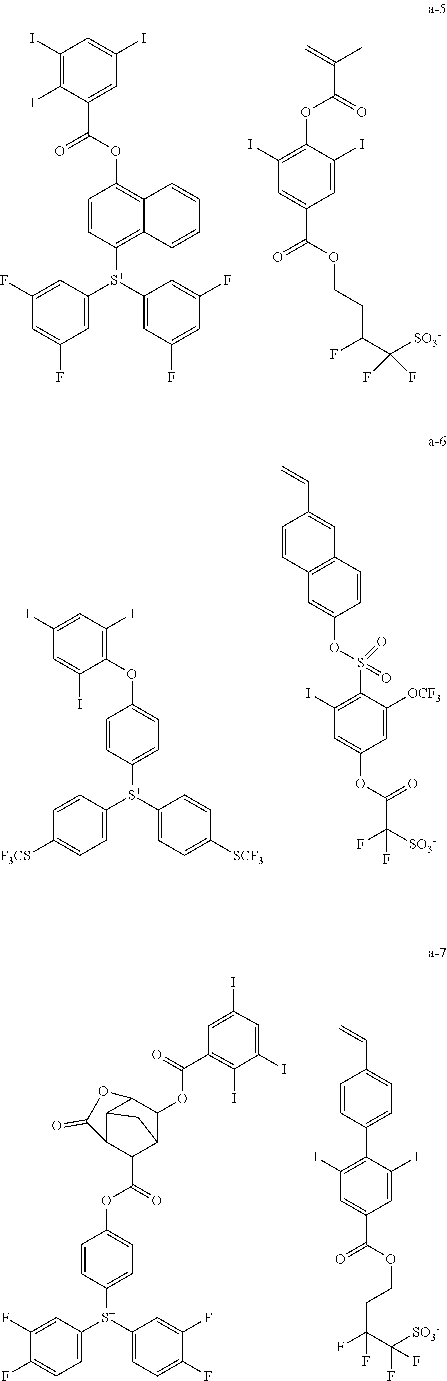

### [Comparative Examples 1-1 to 1-4] Synthesis of Comparative Onium Salt Monomers ca-1 to ca-4

Comparative Onium Salt Monomers ca-1 to ca-4, shown below, were synthesized using the corresponding reactants and well-known organic synthesis reaction.

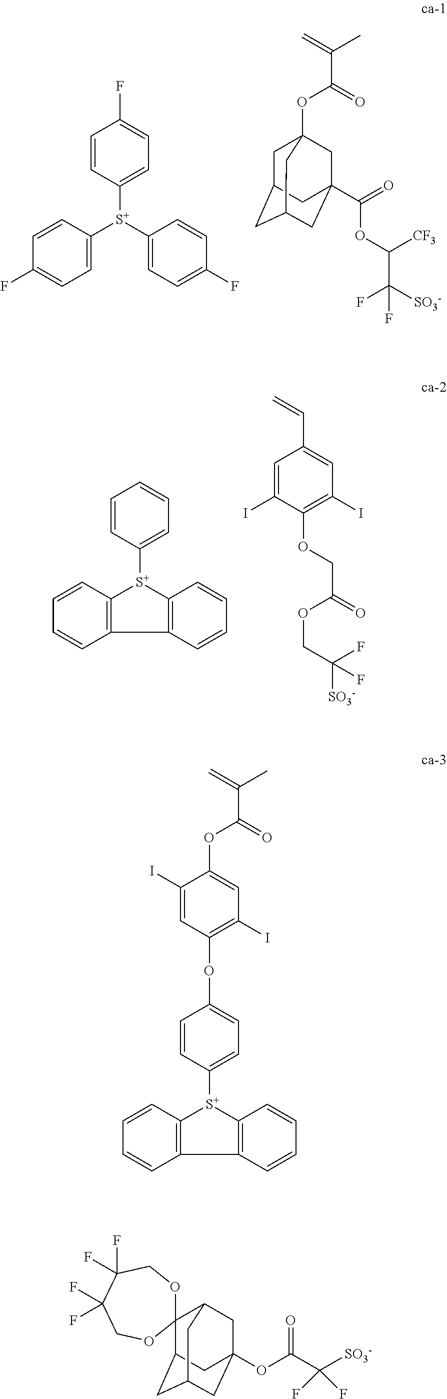

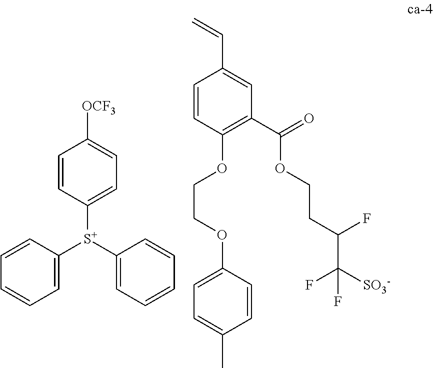

### [2] Synthesis of Base Polymers

Base polymers were synthesized using Monomers a-1 to a-7, Comparative Monomers ca-1 to ca-4, and other monomers as shown below.

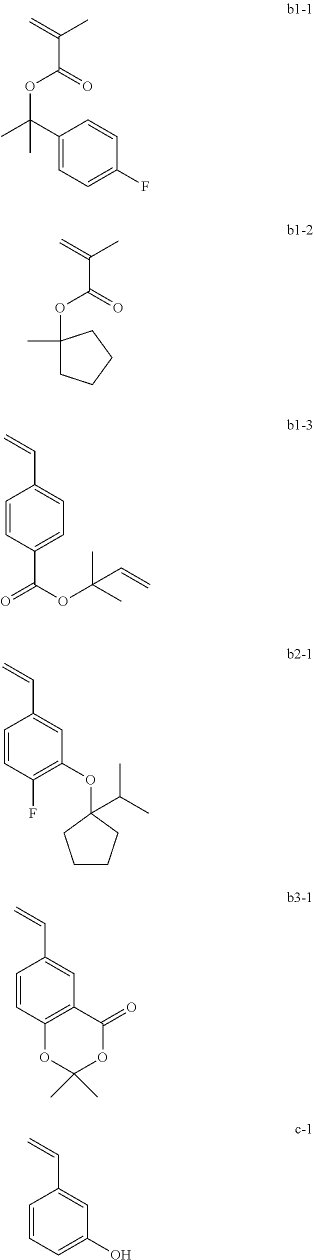

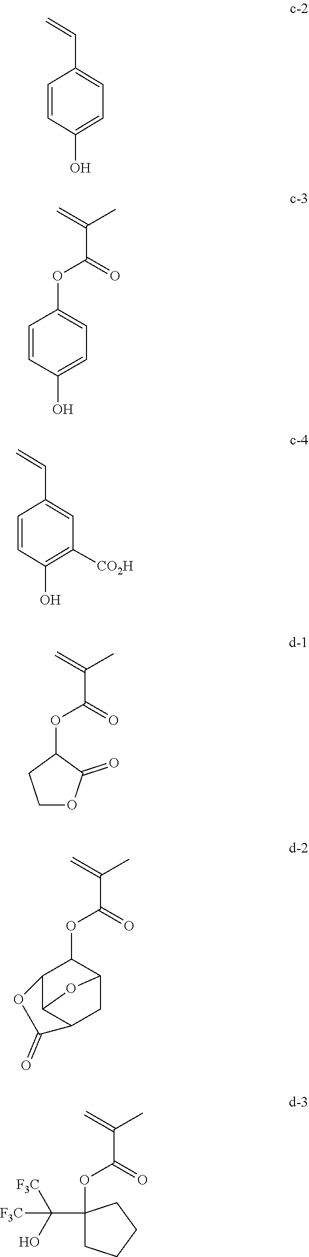

### [Example 2-1] Synthesis of Polymer P-1

A flask under nitrogen atmosphere was charged with 53.8 g of Monomer a-1, 35.8 g of Monomer b1-1, 10.5 g of Monomer c-1, 3.36 g of V-601 (dimethyl 2,2′-azobis(2-methylpropionate) by Fujifilm Wako Pure Chemical Corp.), and 139 g of MEK to form a monomer/initiator solution. Another flask under nitrogen atmosphere was charged with 46 g of MEK, which was heated at 80° C. with stirring. The monomer/initiator solution was added dropwise to the MEK over 4 hours. At the end of addition, the polymerization solution was continuously stirred for 2 hours while maintaining the temperature at 80° C. The polymerization solution was cooled to room temperature, after which it was added dropwise to 3,000 g of hexane with vigorous stirring. The precipitate was collected by filtration. The precipitate was washed twice with 600 g of hexane and vacuum dried at 50° C. for 20 hours, obtaining Polymer P-1 as white powder. Amount 97.1 g, yield 97%. Polymer P-1 had a Mw of 9,400 and a Mw/Mn of 1.75. It is noted that Mw is measured by GPC versus polystyrene standards using DMF solvent.

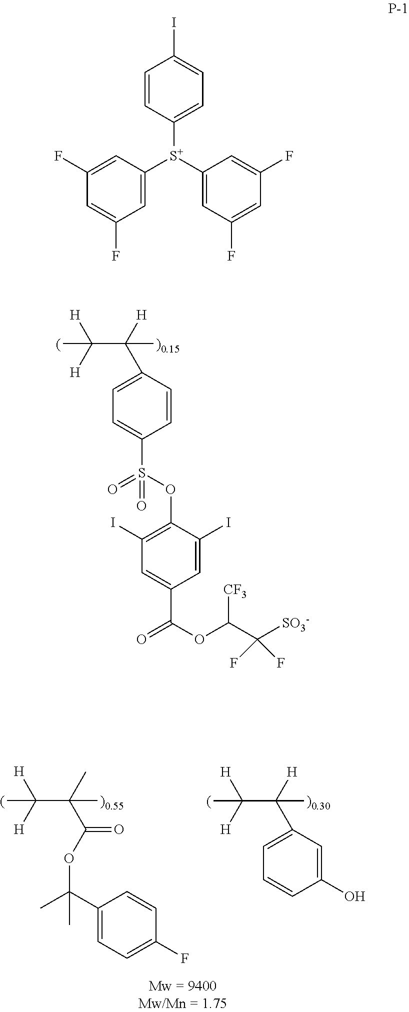

### [Examples 2-2 to 2-20 and Comparative Examples 2-1 to 2-10] Synthesis of Polymers P-2 to P-20 and Comparative Polymers CP-1 to CP-10

Polymers shown in Tables 1 and 2 were synthesized by the same procedure as in Example 2-1 except that the type and amount (blending ratio) of monomers were changed.

TABLE 1

| | | | | Incorporation | | Incorporation | | Incorporation |
|---|---|---|---|---|---|---|---|---|
| | | | Unit | ratio | Unit | ratio | Unit | ratio |
| | | Polymer | 1 | (mol %) | 2 | (mol %) | 3 | (mol %) |
| Example | 2-1 | P-1 | a-1 | 15 | b1-1 | 55 | — | — |
| | 2-2 | P-2 | a-2 | 15 | b1-1 | 55 | — | — |
| | 2-3 | P-3 | a-3 | 15 | b1-1 | 55 | — | — |
| | 2-4 | P-4 | a-4 | 15 | b1-1 | 55 | — | — |
| | 2-5 | P-5 | a-5 | 15 | b1-1 | 55 | — | — |
| | 2-6 | P-6 | a-6 | 15 | b1-1 | 55 | — | — |
| | 2-7 | P-7 | a-7 | 15 | b1-1 | 55 | — | — |
| | 2-8 | P-8 | a-1 | 15 | b1-2 | 55 | — | — |
| | 2-9 | P-9 | a-1 | 15 | b2-1 | 55 | — | — |
| | 2-10 | P-10 | a-2 | 15 | b3-1 | 45 | — | — |
| | 2-11 | P-11 | a-3 | 10 | b1-3 | 35 | b2-1 | 20 |
| | 2-12 | P-12 | a-4 | 15 | b1-1 | 25 | b1-2 | 25 |
| | 2-13 | P-13 | a-5 | 15 | b1-3 | 50 | c-3 | 25 |
| | 2-14 | P-14 | a-6 | 15 | b2-1 | 25 | b3-1 | 25 |
| | 2-15 | P-15 | a-7 | 10 | b1-2 | 35 | b2-1 | 15 |
| | 2-16 | P-16 | a-1 | 15 | b1-1 | 35 | b1-3 | 15 |
| | 2-17 | P-17 | a-2 | 15 | b1-1 | 25 | b3-1 | 15 |
| | 2-18 | P-18 | a-3 | 20 | b1-2 | 45 | c-2 | 30 |
| | 2-19 | P-19 | a-1 | 5 | b1-1 | 50 | c-4 | 25 |
| | 2-20 | P-20 | a-1 | 15 | b1-2 | 30 | b1-3 | 20 |

| | | | | Incorporation | | Incorporation | | |
|---|---|---|---|---|---|---|---|---|
| | | | Unit | ratio | Unit | ratio | | |
| | | Polymer | 4 | (mol %) | 5 | (mol %) | Mw | Mw/Mn |
| Example | 2-1 | P-1 | c-1 | 30 | — | — | 9,400 | 1.75 |
| | 2-2 | P-2 | c-1 | 30 | — | — | 9,300 | 1.76 |
| | 2-3 | P-3 | c-1 | 30 | — | — | 9,100 | 1.75 |
| | 2-4 | P-4 | c-1 | 30 | — | — | 9,200 | 1.76 |
| | 2-5 | P-5 | c-1 | 30 | — | — | 8,800 | 1.74 |
| | 2-6 | P-6 | c-1 | 30 | — | — | 9,100 | 1.76 |
| | 2-7 | P-7 | c-1 | 30 | — | — | 9,200 | 1.73 |
| | 2-8 | P-8 | c-1 | 30 | — | — | 9,000 | 1.75 |
| | 2-9 | P-9 | c-1 | 30 | — | — | 9,200 | 1.76 |
| | 2-10 | P-10 | c-1 | 40 | — | — | 8,900 | 1.75 |
| | 2-11 | P-11 | c-2 | 25 | d-1 | 10 | 9,300 | 1.75 |
| | 2-12 | P-12 | c-2 | 35 | — | — | 9,200 | 1.73 |
| | 2-13 | P-13 | d-2 | 10 | — | — | 9,100 | 1.74 |
| | 2-14 | P-14 | c-4 | 35 | — | — | 9,000 | 1.75 |
| | 2-15 | P-15 | c-2 | 30 | d-3 | 10 | 8,800 | 1.73 |
| | 2-16 | P-16 | c-4 | 35 | — | — | 9,400 | 1.74 |
| | 2-17 | P-17 | c-2 | 30 | d-1 | 15 | 9,300 | 1.75 |
| | 2-18 | P-18 | d-3 | 5 | — | — | 9,100 | 1.74 |
| | 2-19 | P-19 | d-2 | 10 | d-3 | 10 | 9,300 | 1.75 |
| | 2-20 | P-20 | c-2 | 35 | — | — | 9,100 | 1.74 |

TABLE 2

| | | | | Incorporation | | Incorporation | | Incorporation |
|---|---|---|---|---|---|---|---|---|
| | | | Unit | ratio | Unit | ratio | Unit | ratio |
| | | Polymer | 1 | (mol %) | 2 | (mol %) | 3 | (mol %) |
| Comparative | 2-1 | CP-1 | ca-1 | 15 | b1-1 | 55 | — | — |
| Example | 2-2 | CP-2 | ca-2 | 15 | b1-1 | 55 | — | — |
| | 2-3 | CP-3 | ca-3 | 15 | b1-1 | 55 | — | — |
| | 2-4 | CP-4 | ca-4 | 15 | b1-1 | 55 | — | — |
| | 2-5 | CP-5 | ca-1 | 10 | b1-3 | 35 | b2-1 | 20 |
| | 2-6 | CP-6 | ca-2 | 10 | b1-2 | 35 | b2-1 | 15 |
| | 2-7 | CP-7 | ca-3 | 15 | b1-1 | 35 | b1-3 | 15 |
| | 2-8 | CP-8 | ca-4 | 15 | b1-3 | 50 | c-3 | 25 |
| | 2-9 | CP-9 | ca-2 | 15 | b1-1 | 25 | b3-1 | 15 |
| | 2-10 | CP-10 | ca-4 | 5 | b1-1 | 50 | c-4 | 25 |

| | | | | Incorporation | | Incorporation | | |
|---|---|---|---|---|---|---|---|---|
| | | | Unit | ratio | Unit | ratio | | |
| | | Polymer | 4 | (mol %) | 5 | (mol %) | Mw | Mw/Mn |
| Comparative | 2-1 | CP-1 | c-1 | 30 | — | — | 9,300 | 1.73 |
| Example | 2-2 | CP-2 | c-1 | 30 | — | — | 9,200 | 1.75 |
| | 2-3 | CP-3 | c-1 | 30 | — | — | 9,000 | 1.73 |
| | 2-4 | CP-4 | c-1 | 30 | — | — | 8,900 | 1.75 |
| | 2-5 | CP-5 | c-2 | 20 | d-1 | 20 | 9,100 | 1.73 |
| | 2-6 | CP-6 | c-2 | 30 | d-3 | 10 | 9,200 | 1.75 |
| | 2-7 | CP-7 | c-4 | 35 | — | — | 9,300 | 1.74 |
| | 2-8 | CP-8 | d-2 | 10 | — | — | 9,400 | 1.73 |
| | 2-9 | CP-9 | c-2 | 30 | d-1 | 15 | 9,100 | 1.75 |
| | 2-10 | CP-10 | d-2 | 10 | d-3 | 10 | 9,000 | 1.74 |

### [3] Preparation of Resist Composition

### Examples 3-1 to 3-20 and Comparative Examples 3-1 to 3-10

A chemically amplified resist composition (R-1 to R-20, CR-1 to CR-10) was prepared by dissolving an inventive base polymer (P-1 to P-20) or comparative base polymer (CP-1 to CP-10), acid generator (PAG-1, PAG-2), and quencher (SQ-1 to SQ-3, AQ-1) in an organic solvent containing 0.01 wt % of surfactant FC-4430 (3M) in accordance with the formulation shown in Tables 3 and 4, and filtering the solution through a Teflon® filter with a pore size of 0.2 μm.

TABLE 3

| | | | Base | | Acid | | |
|---|---|---|---|---|---|---|---|
| | | Resist | polymer | Quencher | generator | Solvent 1 | Solvent 2 |
| | | composition | (pbw) | (pbw) | (pbw) | (pbw) | (pbw) |
| Example | 3-1 | R-1 | P-1 (80) | SQ-1 (8.0) | — | PGMEA (2200) | DAA (900) |
| | 3-2 | R-2 | P-2 (80) | SQ-1 (7.8) | — | PGMEA (2200) | DAA (900) |
| | 3-3 | R-3 | P-3 (80) | SQ-1 (7.4) | — | PGMEA (2200) | DAA (900) |
| | 3-4 | R-4 | P-4 (80) | SQ-1 (8.0) | — | PGMEA (2200) | DAA (900) |
| | 3-5 | R-5 | P-5 (80) | SQ-1 (8.0) | — | PGMEA (2200) | DAA (900) |
| | 3-6 | R-6 | P-6 (80) | SQ-1 (8.0) | — | PGMEA (2200) | DAA (900) |
| | 3-7 | R-7 | P-7 (80) | SQ-1 (7.8) | — | PGMEA (2200) | DAA (900) |
| | 3-8 | R-8 | P-8 (80) | SQ-2 (8.0) | — | PGMEA (2200) | DAA (900) |
| | 3-9 | R-9 | P-9 (80) | SQ-2 (8.0) | — | PGMEA (2200) | DAA (900) |
| | 3-10 | R-10 | P-10 (80) | SQ-1 (4.0) | — | PGMEA (2200) | DAA (900) |
| | | | | AQ-1 (4.0) | | | |
| | 3-11 | R-11 | P-11 (80) | SQ-3 (7.8) | — | PGMEA (2200) | DAA (900) |
| | 3-12 | R-12 | P-12 (80) | SQ-3 (7.6) | — | PGMEA (2200) | DAA (900) |
| | 3-13 | R-13 | P-13 (80) | SQ-1 (7.8) | — | PGMEA (2200) | DAA (900) |
| | 3-14 | R-14 | P-14 (80) | SQ-3 (8.0) | — | PGMEA (2200) | DAA (900) |
| | 3-15 | R-15 | P-15 (80) | SQ-2 (4.0) | PAG-1 (10) | PGMEA (2200) | DAA (900) |
| | | | | AQ-1 (4.0) | | | |
| | 3-16 | R-16 | P-16 (80) | SQ-1 (8.2) | — | PGMEA (2200) | DAA (900) |
| | 3-17 | R-17 | P-17 (80) | SQ-1 (7.6) | — | PGMEA (2200) | DAA (900) |
| | 3-18 | R-18 | P-18 (80) | SQ-3 (8.0) | — | PGMEA (2200) | DAA (900) |
| | 3-19 | R-19 | P-19 (80) | SQ-3 (4.0) | PAG-2 (15) | PGMEA (2200) | DAA (900) |
| | | | | AQ-1 (4.0) | | | |
| | 3-20 | R-20 | P-20 (80) | SQ-2 (7.6) | — | PGMEA (2200) | DAA (900) |

TABLE 4

| | | | Base | | Acid | | |
|---|---|---|---|---|---|---|---|
| | | Resist | polymer | Quencher | generator | Solvent 1 | Solvent 2 |
| | | composition | (pbw) | (pbw) | (pbw) | (pbw) | (pbw) |
| Comparative | 3-1 | CR-1 | P-1 (80) | SQ-1 (8.0) | — | PGMEA (2200) | DAA (900) |
| Example | 3-2 | CR-2 | P-2 (80) | SQ-1 (7.8) | — | PGMEA (2200) | DAA (900) |
| | 3-3 | CR-3 | P-3 (80) | SQ-1 (7.8) | — | PGMEA (2200) | DAA (900) |
| | 3-4 | CR-4 | P-4 (80) | SQ-1 (8.2) | — | PGMEA (2200) | DAA (900) |
| | 3-5 | CR-5 | P-5 (80) | SQ-3 (7.8) | — | PGMEA (2200) | DAA (900) |
| | 3-6 | CR-6 | P-6 (80) | SQ-2 (4.0) | PAG-1 (10) | PGMEA (2200) | DAA (900) |
| | | | | AQ-1 (4.0) | | | |
| | 3-7 | CR-7 | P-7 (80) | SQ-1 (8.2) | — | PGMEA (2200) | DAA (900) |
| | 3-8 | CR-8 | P-8 (80) | SQ-2 (7.8) | — | PGMEA (2200) | DAA (900) |
| | 3-9 | CR-9 | P-9 (80) | SQ-3 (7.6) | — | PGMEA (2200) | DAA (900) |
| | 3-10 | CR-10 | P-10 (80) | SQ-3 (4.0) | PAG-2 (15) | PGMEA (2200) | DAA (900) |
| | | | | AQ-1 (4.0) | | | |

The components in Tables 3 and 4 are identified below.

### Organic Solvent:

PGMEA: propylene glycol monomethyl ether acetate
DAA: diacetone alcohol

### Quenchers: SQ-1 to SQ-3 and AQ-1

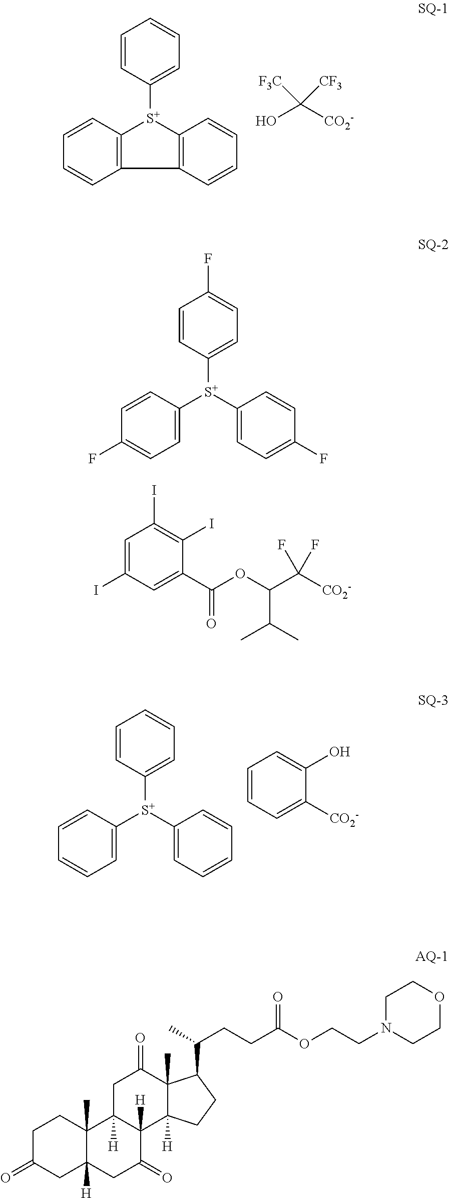

### Acid Generators: PAG-1 and PAG-2

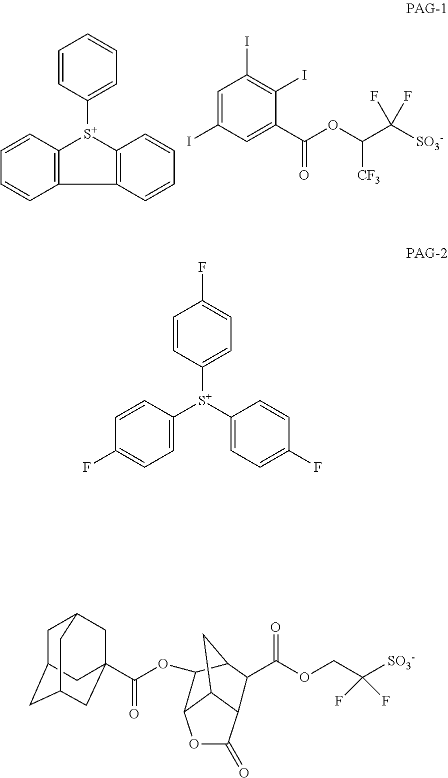

### [4] EUV Lithography Test 1

### Examples 4-1 to 4-20 and Comparative Examples 4-1 to 4-10

Each of the chemically amplified resist compositions (R-1 to R-20, CR-1 to CR-10 in Tables 3 and 4) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 100° C. for 60 seconds to form a resist film of 50 nm thick. Using an EUV scanner NXE3300 (ASML, NA 0.33, σ 0.9/0.6, dipole illumination), the resist film was exposed to EUV through a mask bearing a line-and-space (LS) pattern having a width of 18 nm and a pitch of 36 nm (on-wafer size) while changing the dose at a pitch of 1 mJ/cm² and the focus at a pitch of 0.020 μm. The resist film was baked (PEB) at the temperature shown in Tables 5 and 6 for 60 seconds. This was followed by puddle development in a 2.38 wt % TMAH aqueous solution for 30 seconds, rinsing with a surfactant-containing rinse fluid, and spin drying. A positive LS pattern was obtained.

The LS pattern was observed under CD-SEM (CG6300, Hitachi High-Technologies Corp.) and evaluated for sensitivity, exposure latitude (EL), LWR, depth of focus (DOF), and collapse limit by the following methods. The results are shown in Tables 5 and 6.

### [Evaluation of Sensitivity]

The optimum dose Eop (mJ/cm²) which provided an LS pattern with a line width of 18 nm and a pitch of 36 nm was determined and reported as sensitivity. A smaller value indicates a higher sensitivity.

### [Evaluation of EL]

The exposure dose which provided a LS pattern with a space width of 18 nm±10% (i.e., 16.2 to 19.8 nm) was determined. EL (%) is calculated from the exposure doses according to the following equation:

EL (%)=(|E₁−E₂|/Eop)×100
wherein E₁ is an optimum exposure dose which provides a LS pattern with a line width of 16.2 nm and a pitch of 36 nm, E₂ is an optimum exposure dose which provides a LS pattern with a line width of 19.8 nm and a pitch of 36 nm, and Eop is an optimum exposure dose which provides a LS pattern with a line width of 18 nm and a pitch of 36 nm. A larger value indicates better performance.

### [Evaluation of LWR]

For the LS pattern formed by exposure at the optimum dose Eop, the line width was measured at 10 longitudinally spaced apart points, from which a 3-fold value (36) of the standard deviation (6) was determined and reported as LWR. A smaller value of 36 indicates a pattern having small roughness and uniform line width.

### [Evaluation of DOF]

As an index of DOF, a range of focus which provided a LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 5

| | | | PEB | | | | | Collapse |
|---|---|---|---|---|---|---|---|---|
| | | Resist | temp. | Eop | EL | LWR | DOF | limit |
| | | composition | (° C.) | (mJ/cm²) | (%) | (nm) | (nm) | (nm) |
| Example | 4-1 | R-1 | 100 | 33 | 19 | 2.5 | 120 | 10.7 |
| | 4-2 | R-2 | 95 | 33 | 17 | 2.6 | 110 | 10.9 |
| | 4-3 | R-3 | 100 | 34 | 18 | 2.7 | 120 | 10.8 |
| | 4-4 | R-4 | 105 | 33 | 19 | 2.6 | 110 | 10.8 |
| | 4-5 | R-5 | 100 | 33 | 17 | 2.5 | 100 | 11.2 |
| | 4-6 | R-6 | 95 | 34 | 18 | 2.7 | 110 | 11.4 |
| | 4-7 | R-7 | 100 | 34 | 18 | 2.6 | 110 | 10.9 |
| | 4-8 | R-8 | 100 | 33 | 19 | 2.7 | 120 | 11.3 |
| | 4-9 | R-9 | 105 | 32 | 19 | 2.5 | 110 | 11.5 |
| | 4-10 | R-10 | 100 | 33 | 18 | 2.7 | 120 | 11.3 |
| | 4-11 | R-11 | 100 | 34 | 19 | 2.7 | 110 | 11.1 |
| | 4-12 | R-12 | 100 | 34 | 17 | 2.6 | 120 | 11.2 |
| | 4-13 | R-13 | 105 | 35 | 18 | 2.5 | 120 | 10.9 |
| | 4-14 | R-14 | 100 | 34 | 17 | 2.7 | 100 | 11.4 |
| | 4-15 | R-15 | 100 | 33 | 19 | 2.5 | 110 | 11.2 |
| | 4-16 | R-16 | 105 | 34 | 18 | 2.7 | 120 | 10.9 |
| | 4-17 | R-17 | 95 | 35 | 17 | 2.5 | 110 | 10.8 |
| | 4-18 | R-18 | 100 | 34 | 19 | 2.7 | 110 | 11.2 |
| | 4-19 | R-19 | 100 | 33 | 18 | 2.7 | 120 | 11.4 |
| | 4-20 | R-20 | 100 | 34 | 17 | 2.5 | 110 | 11.2 |

TABLE 6

| | | | PEB | | | | | Collapse |
|---|---|---|---|---|---|---|---|---|
| | | Resist | temp. | Eop | EL | LWR | DOF | limit |
| | | composition | (° C.) | (mJ/cm²) | (%) | (nm) | (nm) | (nm) |
| Comparative | 4-1 | CR-1 | 105 | 40 | 14 | 3.6 | 90 | 13.7 |
| Example | 4-2 | CR-2 | 100 | 38 | 14 | 3.1 | 90 | 12.6 |
| | 4-3 | CR-3 | 100 | 37 | 11 | 4.1 | 70 | 12.5 |
| | 4-4 | CR-4 | 100 | 38 | 14 | 3.1 | 90 | 12.4 |
| | 4-5 | CR-5 | 105 | 41 | 14 | 3.5 | 80 | 13.3 |
| | 4-6 | CR-6 | 95 | 38 | 13 | 3.2 | 100 | 12.6 |
| | 4-7 | CR-7 | 100 | 37 | 10 | 3.9 | 70 | 12.5 |
| | 4-8 | CR-8 | 100 | 37 | 14 | 3.3 | 90 | 12.8 |
| | 4-9 | CR-9 | 105 | 37 | 14 | 3.1 | 90 | 12.4 |
| | 4-10 | CR-10 | 100 | 38 | 15 | 3 | 90 | 12.6 |

As seen from Tables 5 and 6, resist compositions comprising polymers comprising repeat units derived from onium salt monomers within the scope of the invention exhibit a high sensitivity and improved lithography properties including EL, LWR and DOF. Small values of collapse limit show that small-size patterns have resistance to collapse. It is demonstrated that the chemically amplified resist compositions within the scope of the invention are suited for the EUV lithography process.

### [5] EUV Lithography Test 2

### Examples 5-1 to 5-20 and Comparative Examples 5-1 to 5-10

Each of the chemically amplified resist compositions (R-1 to R-20, CR-1 to CR-10 in Tables 3 and 4) was spin coated on a silicon substrate having a 20-nm coating of silicon-containing spin-on hard mask SHB-A940 (Shin-Etsu Chemical Co., Ltd., silicon content 43 wt %) and prebaked on a hotplate at 105° C. for 60 seconds to form a resist film of 50 nm thick. Using an EUV scanner NXE3400 (ASML, NA 0.33, σ0.9/0.6, quadrupole illumination), the resist film was exposed to EUV through a mask bearing a hole pattern having a pitch of 46 nm+20% bias (on-wafer size). The resist film was baked (PEB) on a hotplate at the temperature shown in Tables 7 and 8 for 60 seconds. This was followed by development in a 2.38 wt % TMAH aqueous solution for 30 seconds. Hole patterns with a size of 23 nm were obtained.

The hole pattern was observed under CD-SEM (CG6300, Hitachi High-Technologies Corp.). The exposure dose Eop that provides a hole pattern having a size of 23 nm was determined and reported as sensitivity. The size of 50 holes at that dose was measured, from which a 3-fold value (36) of the standard deviation (6) was computed and reported as CDU. The results are also shown in Tables 7 and 8.

TABLE 7

| | | Resist | PEB temp. | Eop | CDU |
|---|---|---|---|---|---|
| | | composition | (° C.) | (mJ/cm²) | (nm) |
| Example | 5-1 | R-1 | 95 | 21 | 2.1 |
| | 5-2 | R-2 | 95 | 22 | 2.2 |
| | 5-3 | R-3 | 95 | 23 | 2.3 |
| | 5-4 | R-4 | 95 | 21 | 2.4 |
| | 5-5 | R-5 | 90 | 22 | 2.3 |
| | 5-6 | R-6 | 90 | 23 | 2.3 |
| | 5-7 | R-7 | 95 | 21 | 2.5 |
| | 5-8 | R-8 | 95 | 22 | 2.3 |
| | 5-9 | R-9 | 90 | 23 | 2.2 |
| | 5-10 | R-10 | 95 | 22 | 2.3 |
| | 5-11 | R-11 | 95 | 24 | 2.2 |
| | 5-12 | R-12 | 95 | 21 | 2.3 |
| | 5-13 | R-13 | 90 | 23 | 2.3 |
| | 5-14 | R-14 | 95 | 23 | 2.3 |
| | 5-15 | R-15 | 90 | 22 | 2.4 |
| | 5-16 | R-16 | 90 | 22 | 2.2 |
| | 5-17 | R-17 | 95 | 21 | 2.1 |
| | 5-18 | R-18 | 90 | 23 | 2.3 |
| | 5-19 | R-19 | 90 | 23 | 2.3 |
| | 5-20 | R-20 | 95 | 22 | 2.2 |

TABLE 8

| | | Resist | PEB temp. | Eop | CDU |
|---|---|---|---|---|---|
| | | composition | (° C.) | (mJ/cm²) | (nm) |
| Comparative | 5-1 | CR-1 | 95 | 29 | 3.3 |
| Example | 5-2 | CR-2 | 95 | 27 | 2.7 |
| | 5-3 | CR-3 | 95 | 26 | 3.3 |
| | 5-4 | CR-4 | 90 | 28 | 2.7 |
| | 5-5 | CR-5 | 90 | 30 | 3.1 |
| | 5-6 | CR-6 | 95 | 27 | 2.9 |
| | 5-7 | CR-7 | 90 | 26 | 3.4 |
| | 5-8 | CR-8 | 90 | 28 | 2.8 |
| | 5-9 | CR-9 | 90 | 26 | 2.7 |
| | 5-10 | CR-10 | 95 | 27 | 2.8 |

It is demonstrated in Tables 7 and 8 that chemically amplified resist compositions within the scope of the invention exhibit a high sensitivity and improved CDU.

### [6] Dry Etching Test

### Examples 6-1 to 6-20 and Comparative Examples 6-1 to 6-10

Each of the polymers (Polymers P-1 to P-20, Comparative Polymers CP-1 to CP-10 in Tables 1 and 2), 2 g, was thoroughly dissolved in 10 g of cyclohexanone, and passed through a filter having a pore size of 0.2 μm, obtaining a polymer solution. The polymer solution was spin coated onto a silicon substrate and baked to form a polymer film of 300 nm thick. Using a dry etching instrument TE-8500P (Tokyo Electron Ltd.), the polymer film was etched with CHF₃/CF₄ gas under the following conditions.
Chamber pressure: 40 Pa
RF power: 1000 W
Gap: 9 mm
CHF₃ gas flow rate: 30 ml/min
CF₄ gas flow rate: 30 ml/min
Ar gas flow rate: 100 ml/min
Time: 60 sec

The difference in polymer film thickness before and after etching was determined, from which an etching rate per minute was computed. The results are shown in Tables 9 and 10. A smaller value of film thickness difference, i.e., a lower etching rate indicates better etch resistance.

TABLE 9

| | | Polymer | CHF₃/CF₄ gas etching rate (nm/min) |
|---|---|---|---|
| Example | 6-1 | P-1 | 94 |
| | 6-2 | P-2 | 96 |
| | 6-3 | P-3 | 95 |
| | 6-4 | P-4 | 94 |
| | 6-5 | P-5 | 95 |
| | 6-6 | P-6 | 94 |
| | 6-7 | P-7 | 95 |
| | 6-8 | P-8 | 96 |
| | 6-9 | P-9 | 94 |
| | 6-10 | P-10 | 97 |
| | 6-11 | P-11 | 96 |
| | 6-12 | P-12 | 95 |
| | 6-13 | P-13 | 94 |
| | 6-14 | P-14 | 96 |
| | 6-15 | P-15 | 98 |
| | 6-16 | P-16 | 96 |
| | 6-17 | P-17 | 97 |
| | 6-18 | P-18 | 95 |
| | 6-19 | P-19 | 94 |
| | 6-20 | P-20 | 95 |

TABLE 10

| | | Polymer | CHF₃/CF₄ gas etching rate (nm/min) |
|---|---|---|---|
| Comparative | 6-1 | CP-1 | 109 |
| Example | 6-2 | CP-2 | 101 |
| | 6-3 | CP-3 | 103 |
| | 6-4 | CP-4 | 100 |
| | 6-5 | CP-5 | 110 |
| | 6-6 | CP-6 | 102 |
| | 6-7 | CP-7 | 101 |
| | 6-8 | CP-8 | 103 |
| | 6-9 | CP-9 | 104 |
| | 6-10 | CP-10 | 103 |

It is evident from Tables 9 and 10 that the inventive polymers have good dry etch resistance, i.e., resistance to CHF₃/CF₄ gas etching.

Japanese Patent Application No. 2024-012406 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. An onium salt monomer consisting of an anion having the formula (a1) and a cation having the formula (a2): 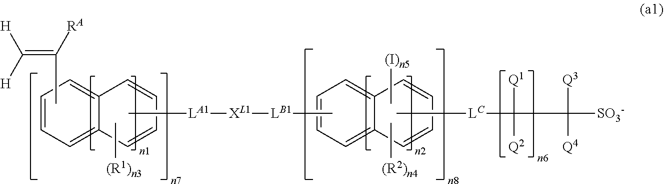
wherein n1 is 0 or 1, n2 is 0 or 1, n3 is an integer of 0 to 4, n4 is an integer of 0 to 4, n5 is an integer of 1 to 6, n4+n5 is from 1 to 4 when n2=0, n4+n5 is from 1 to 6 when n2=1, n6 is an integer of 0 to 4, n7 is 0 or 1, n8 is 0 or 1, excluding that n7 and n8 are 0 at the same time,
R^{A} is hydrogen, fluorine, methyl or trifluoromethyl,
R¹ is halogen, nitro group, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom, with the proviso that when n3=2, 3 or 4 and n7=1, a plurality of R¹ may be identical or different and a plurality of R¹ may bond together to form a ring with the carbon atoms to which they are attached, and that when n8=0, n7 is 1 and n3 is 1, 2, 3 or 4, and at least one of R¹ is iodine,
R² is halogen exclusive of iodine, nitro group, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom, with the proviso that a plurality of R² may be identical or different and a plurality of R² may bond together to form a ring with the carbon atoms to which they are attached, when n4=2, 3 or 4 and n8=1,
L^{A1}, L^{B1} and L^{C} are each independently a single bond, ether bond, ester bond, sulfonate ester bond, amide bond, sulfonamide bond, carbonate bond or carbamate bond,
X^{L1} is a single bond or a C₁-C₄₀ hydrocarbylene group which may contain a heteroatom,
Q¹ and Q² are each independently hydrogen, fluorine or a C₁-C₆ fluorinated saturated hydrocarbyl group,
Q³ and Q⁴ are each independently fluorine or a C₁-C₆ fluorinated saturated hydrocarbyl group, 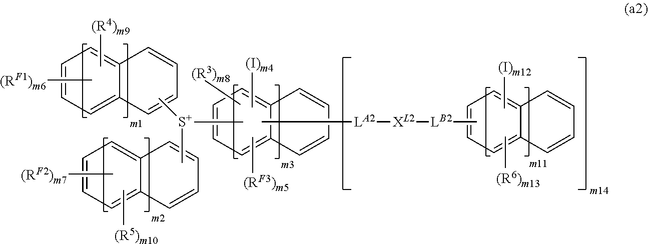
wherein m1 is 0 or 1, m2 is 0 or 1, m3 is 0 or 1, m4 is an integer of 0 to 4, m5 is an integer of 0 to 4, m6 is an integer of 0 to 6, m7 is an integer of 0 to 6, m8 is an integer of 0 to 2, m9 is an integer of 0 to 2, m1 is an integer of 0 to 2, m1 is 0 or 1, m12 is an integer of 0 to 4, m13 is an integer of 0 to 2, m14 is an integer of 0 to 2, m6+m9 is from 0 to 4 when m1=0, m6+m9 is from 0 to 6 when m1=1, m7+m10 is from 0 to 4 when m2=0, m7+m10 is from 0 to 6 when m2=1, m4+m5+m8+m14 is from 1 to 4 when m3=0, m4+m5+m8+m14 is from 1 to 6 when m3=1, m12+m13 is from 0 to 4 when m11=, m12+m13 is from 0 to 6 when m11=1, m4+m12 is 1 or greater,
R^{F1} to R^{F3} are each independently fluorine, a C₁-C₆ fluorinated saturated hydrocarbyl group, C₁-C₆ fluorinated saturated hydrocarbyloxy group, or C₁-C₆ fluorinated saturated hydrocarbylthio group,
with the proviso that a plurality of R^{F1} may be identical or different when m6 is 2 or more, a plurality of R^{F2} may be identical or different when m7 is 2 or more, and a plurality of R^{F3} may be identical or different when m5 is 2 or more,
R³ to R⁶ are each independently halogen exclusive of iodine and fluorine, nitro, cyano, a C₁-C₂₀ hydrocarbyl group which may contain a heteroatom, C₁-C₂₀ hydrocarbyloxy group which may contain a heteroatom, or C₁-C₂₀ hydrocarbylthio group which may contain a heteroatom, with the proviso that two R³ may be identical or different and two R³ may bond together to form a ring with the carbon atoms to which they are attached, when m8=2, two R⁴ may be identical or different and two R⁴ may bond together to form a ring with the carbon atoms to which they are attached, when m9=2, two R⁵ may be identical or different and two R⁵ may bond together to form a ring with the carbon atoms to which they are attached, when m10=2, two R⁶ may be identical or different and two R⁶ may bond together to form a ring with the carbon atoms to which they are attached, when m13=2,
the aromatic rings directly bonded to S⁺ in the sulfonium cation may bond together to form a ring with S⁺,
L^{A2} and L^{B2} are each independently a single bond, ether bond, ester bond, amide bond, sulfonate ester bond, sulfonamide bond, carbonate bond or carbamate bond, and
X^{L2} is a single bond or a C₁-C₄₀ hydrocarbylene group which may contain a heteroatom.

2. The monomer of claim 1 wherein the anion has the formula (a1-1): 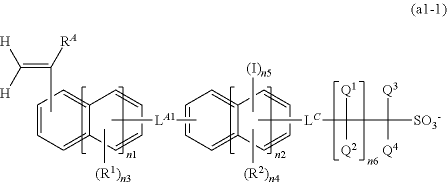
wherein n1 to n6, R^{A}, R¹, R², L^{A1}, L^{C} and Q¹ to Q⁴ are as defined above.

3. The monomer of claim 2 wherein the anion has the formula (a1-2): 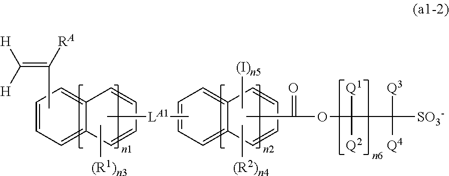
wherein n1 to n6, R^{A}, R¹, R², L^{A1} and Q¹ to Q⁴ are as defined above.

4. The monomer of claim 1 wherein the cation has the formula (a2-1): 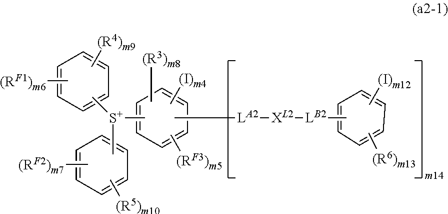
wherein m4 to m10, m12 to m14, R^{F1} to R^{F3}, R³ to R⁶, L^{A2}, L^{B2} and X^{L2} are as defined above.

5. The monomer of claim 4 wherein the cation has the formula (a2-2): 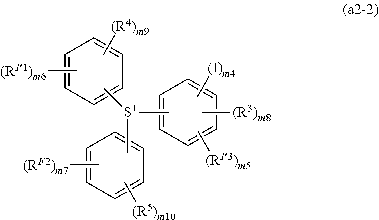
wherein m4 to m10, R^{F1} to R^{F3}, and R³ to R⁵ are as defined above.

6. A monomeric photoacid generator comprising the onium salt monomer of claim 1.

7. A polymer comprising repeat units derived from the monomeric photoacid generator of claim 6.

8. The polymer of claim 7, further comprising repeat units having the formula (b1) or (b2): 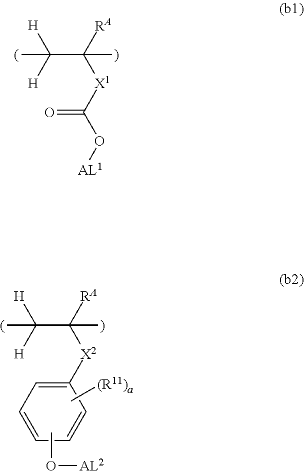
wherein R^{A} is each independently hydrogen, fluorine, methyl or trifluoromethyl,
X¹ is a single bond, phenylene group, naphthylene group, *—C(═O)—O—X¹¹— or *—C(═O)—NH—X¹¹—, the phenylene and naphthylene groups may be substituted with hydroxy, nitro, cyano, a C₁-C₁₀ alkoxy moiety which may contain a heteroatom, or halogen, X¹¹ is a C₁-C₁₀ saturated hydrocarbylene group, phenylene group or naphthylene group, the saturated hydrocarbylene group may contain hydroxy, ether bond, ester bond or lactone ring,
X² is a single bond, *—C(═O)—O— or *—C(═O)—NH—,
*designates a point of attachment to the carbon atom in the backbone,
R¹¹ is halogen, cyano, hydroxy, nitro, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom,
AL¹ and AL² are each independently an acid labile group, and
a is an integer of 0 to 4.

9. The polymer of claim 7, further comprising repeat units having the formula (b3): 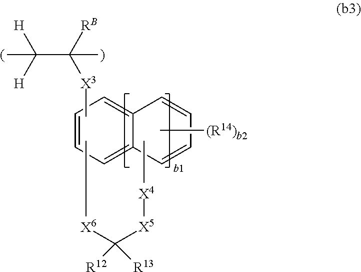
wherein b1 is 0 or 1, b2 is an integer of 0 to 3 when b1=0 and an integer of 0 to 5 when b1=1,
R^{A} is hydrogen, fluorine, methyl or trifluoromethyl,
X³ is a single bond, *—C(═O)—O— or *—C(═O)—NH—, * designates a point of attachment to the carbon atom in the backbone,
R¹² and R¹³ are each independently hydrogen or a C₁-C₂₀ hydrocarbyl group which may contain a heteroatom, R¹² and R¹³ may bond together to form a ring with the carbon atom to which they are attached,
R¹⁴ is halogen, hydroxy, cyano, nitro, a C₁-C₂₀ hydrocarbyl group which may contain a heteroatom, C₁-C₂₀ hydrocarbyloxy group which may contain a heteroatom, C₂-C₂₀ hydrocarbyloxycarbonyl group which may contain a heteroatom, C₁-C₂₀ hydrocarbylthio group which may contain a heteroatom, or —N(R^{14A})(R^{14B}), R^{14A} and R^{14B} are each independently hydrogen or a C₁-C₆ hydrocarbyl group, a plurality of R¹⁴ may bond together to form a ring with the carbon atoms on the aromatic ring to which they are attached, when b2 is 2 or 3,
X⁴ is a single bond, C₁-C₄ aliphatic hydrocarbylene group, carbonyl group, sulfonyl group or a group obtained by combining the foregoing,
X⁵ and X⁶ are each independently oxygen or sulfur, with the proviso that X⁴ and X⁶ are attached to adjacent carbon atoms on the aromatic ring.

10. The polymer of claim 7, further comprising repeat units having the formula (c1): 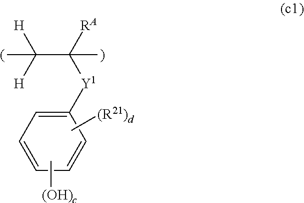
wherein R^{A} is hydrogen, fluorine, methyl or trifluoromethyl,
Y¹ is a single bond, *—C(═O)—O— or *—C(═O)—NH—, * designates a point of attachment to the carbon atom in the backbone,
R²¹ is halogen, nitro, cyano, carboxy, a C₁-C₂₀ hydrocarbyl group which may contain a heteroatom, C₁-C₂₀ hydrocarbyloxy group which may contain a heteroatom, C₂-C₂₀ hydrocarbylcarbonyl group which may contain a heteroatom, C₂-C₂₀ hydrocarbylcarbonyloxy group which may contain a heteroatom, or C₂-C₂₀ hydrocarbyloxycarbonyl group which may contain a heteroatom,
c is an integer of 1 to 4, d is an integer of 0 to 3, and c+d is from 1 to 5.

11. The polymer of claim 7, further comprising repeat units having the formula (dl): 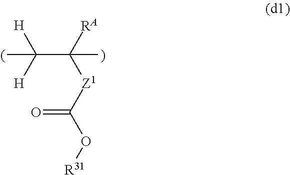
wherein R^{A} is hydrogen, fluorine, methyl or trifluoromethyl,
Z¹ is a single bond, phenylene group, naphthylene group, *—C(═O)—O—Z¹¹— or *—C(═O)—NH—Z¹¹—, the phenylene and naphthylene groups may be substituted with hydroxy, nitro, cyano, a C₁-C₁₀ alkoxy moiety which may contain a heteroatom, or halogen, * designates a point of attachment to the carbon atom in the backbone, Z¹¹ is a C₁-C₁₀ saturated hydrocarbylene group, phenylene group or naphthylene group, the saturated hydrocarbylene group may contain hydroxy, ether bond, ester bond or lactone ring,
R³¹ is hydrogen or a C₁-C₂₀ group containing at least one structure selected from hydroxy other than phenolic hydroxy, cyano, carbonyl, carboxy, ether bond, ester bond, sulfonate ester bond, carbonate bond, lactone ring, sultone ring, and carboxylic anhydride (—C(═O)—O—C(═O)—).

12. A chemically amplified resist composition comprising (A) a base polymer containing the polymer of claim 7.

13. The resist composition of claim 12, further comprising (B) an organic solvent.

14. The resist composition of claim 12, further comprising (C) a quencher.

15. The resist composition of claim 12, further comprising (D) an acid generator.

16. The resist composition of claim 12, further comprising (E) a surfactant.

17. The resist composition of claim 12, further comprising (F) a dissolution inhibitor.

18. A pattern forming process comprising the steps of applying the chemically amplified resist composition of claim 12 onto a substrate to form a resist film thereon, exposing the resist film to high-energy radiation, and developing the exposed resist film in a developer.

19. The process of claim 18 wherein the high-energy radiation is ArF excimer laser of wavelength 193 nm, KrF excimer laser of wavelength 248 nm, EB or EUV of wavelength 3 to 15 nm.
